(19) United States
(12) Patent Application Publication
(10) Pub. No.: US 2023/0193279 A1
(43) Pub. Date: Jun. 22, 2023

(54) Compositions and Methods for Modifying Target RNAs

(71) Applicants: Brian BRIGGS, Seattle, WA (US); Brian BOOTH, Seattle, WA (US); Shape Therapeutics Inc., Seattle, WA (US)

(72) Inventors: Adrian BRIGGS, Seattle, WA (US); Brian BOOTH, West Roxbury, MA (US); Debojit BOSE, Seattle, WA (US); David HUSS, Seattle, WA (US); Yiannis SAVVA, Seattle, WA (US); Richard SULLIVAN, Seattle, WA (US)

(21) Appl. No.: 17/925,871

(22) PCT Filed: May 26, 2021
(86) PCT No.: PCT/US2021/034323

§ 371 Date: Nov. 17, 2022

Related U.S. Application Data
(60) Provisional application No. 63/030,166, filed on May 26, 2020.
(60) Provisional application No. 63/112,329, filed on Nov. 11, 2020.
(60) Provisional application No. 63/119,921, filed on Dec. 1, 2020.
(60) Provisional application No. 63/153,175, filed on Feb. 24, 2021.
(60) Provisional application No. 63/178,059, filed on Apr. 22, 2021.

(51) Int. Cl. C12N 15/113; C12N 15/86; A61K 38/17; A61P 25/16; A61P 29/00; A61P 1/00; A61K 45/06; A61K 31/7088

(52) U.S. Cl. CPC C12N 15/1137; C12Y 207/11001; C12N 15/86; A61K 38/1716; A61P 25/16; A61P 29/00; A61P 1/00; A61K 45/06; A61K 31/7088; C12N 2310/11; C12N 2310/531; C12N 2310/533; C12N 2750/14143; C12N 2750/14171; A61K 48/00

(57) ABSTRACT

Provided herein are compositions and methods that can be utilized to ameliorate, treat, or at least partially eliminate diseases and conditions that can arise from genomic mutations. Subject compositions and methods can be used to edit RNA to ameliorate, treat, or at least partially eliminate the disease and conditions in a subject.

This application claims priority under 35 U.S.C. § 119 from Provisional Application Ser. No. 63/030,166, filed May 26, 2020, Provisional Application Ser. No. 63/112,329, filed Nov. 11, 2020, Provisional Application Ser. No. 63/119,921, filed Dec. 1, 2020, Provisional Application Ser. No. 63/153,175, filed Feb. 24, 2021, and Provisional Application Ser. No. 63/178,059, filed Apr. 22, 2021, the disclosures of which are incorporated herein by reference.

### SUMMARY

Disclosed herein are engineered polynucleotides comprising a targeting sequence that is at least partially complementary to a region of a target RNA, wherein the target RNA: (a) encodes for a Leucine-rich repeat kinase 2 (LRRK2) polypeptide; (b) comprises a non-coding sequence; or (c) comprises (a) and (b), wherein the engineered polynucleotide is configured upon binding to the region of the target RNA, in association with the target RNA, to form a structural feature which recruits an RNA editing entity, wherein the RNA editing entity, when associated with the engineered polynucleotide and the region of the target RNA, facilitates: an editing of a base of a nucleotide in the region of the target RNA, a modulation of translation of the LRRK2 polypeptide, or both. In some embodiments, the targeting sequence is about: 40, 45, 60, 80, 100, 120, 200, or 300 nucleotides in length. In some embodiments, the targeting sequence is about 100 nucleotides in length. In some embodiments, the targeting sequence that is at least partially complementary to the region of the target RNA comprises at least one nucleotide that is not complementary to a nucleotide in the region of the target RNA. In some embodiments, the nucleotide that is not complementary is an adenosine (A) in the region of the target RNA, and wherein the A is comprised in an A/C mismatch. In some embodiments, the nucleotide that is not complementary is an adenosine (A) in the region of the target RNA, and wherein the A is comprised in an internal loop or bulge. In some embodiments, the A is the base of the nucleotide in the region of the target RNA for editing. In some embodiments, the target RNA is selected from the group comprising: an mRNA, a pre-mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA. In some embodiments, the target RNA is an mRNA. In some embodiments, the structural feature comprises: a bulge, a hairpin, an internal loop, and any combination thereof. In some embodiments, the structural feature comprises a bulge. In some embodiments, the bulge is an asymmetric bulge. In some embodiments, the bulge is a symmetric bulge. In some embodiments, the bulge is from 1-4 nucleotides in length. In some embodiments, the structural feature comprises a hairpin. In some embodiments, the structural feature comprises an internal loop. In some embodiments, the internal loop is from 5-50 nucleotides in length. In some embodiments, the internal loop is 6 nucleotides in length. In some embodiments, the engineered polynucleotide comprises at least two internal loops. In some embodiments, the two internal loops are internal symmetrical loops. In some embodiments, the two internal loops are internal symmetrical loops and each side of the two internal loop is 6 nucleotides in length. In some embodiments, the internal loop is an asymmetrical internal loop. In some embodiments, the engineered polynucleotide comprises a structured motif. In some embodiments, the structured motif comprises at least two of: the bulge, the hairpin, and the internal loop. In some embodiments, the structured motif comprises the bulge and the hairpin. In some embodiments, the structured motif comprises the bulge and the internal loop. In some embodiments, the engineered polynucleotide lacks a recruiting domain. In some embodiments, the RNA editing entity comprises an adenosine deaminase acting on RNA (ADAR) polypeptide or biologically active fragment thereof or adenosine deaminases acting on tRNA (ADAT) polypeptide or biologically active fragment thereof. In some embodiments, the ADAR polypeptide or biologically active fragment thereof comprises ADAR1 or ADAR2. In some embodiments, the engineered polynucleotide further comprises an RNA editing entity recruiting domain that is capable of recruiting the RNA editing entity. In some embodiments, the RNA editing entity recruiting domain is at least 1 to about 75 nucleotides in length. In some embodiments, the RNA editing entity recruiting domain is at least 30-50 nucleotides in length. In some embodiments, the RNA editing entity recruiting domain comprises a glutamate ionotropic receptor AMPA type subunit 2 (GluR2) sequence. In some embodiments, the GluR2 sequence comprises at least about 80%, 85%, 90%, 95%, or 99% sequence identity to SEQ ID NO: 1. In some embodiments, the GluR2 sequence comprises SEQ ID NO: 1. In some embodiments, the region is from 5 to 600 nucleotides in length of the target RNA, 40 to 400 nucleotides in length, or 80 to 120 nucleotides in length. In some embodiments, the region is from 50 to 200 nucleotides in length of the target RNA. In some embodiments, the region is about 100 nucleotides in length of the target RNA. In some embodiments, the region of the target RNA comprises at least 60%, 70%, 80%, 85%, 90%, 95%, 97%, 99%, or 100% sequence identity to SEQ ID NO: 73 or SEQ ID NO: 74. In some embodiments, the non-coding sequence comprises a three prime untranslated region (3′ UTR). In some embodiments, the non-coding sequence comprises a five prime untranslated region (5′ UTR). In some embodiments, the editing of the base in the 5′UTR of the region of the target RNA results in at least partially regulating gene translation of the LRRK2 polypeptide. In some embodiments, the editing of the base in the 5′UTR of the region of the target RNA results in facilitating regulation mRNA translation of: the LRRK2 polypeptide. In some embodiments, the target RNA encodes the LRRK2 polypeptide. In some embodiments, the target RNA that encodes the LRRK2 polypeptide comprises at least a portion of: a poly(A) tail, a microRNA response element (MRE), AU-rich element (ARE), hnRNP binding sites or any combination thereof. In some embodiments, the engineered polynucleotide is configured to modulate expression of the LRRK2 polypeptide. In some embodiments, the target RNA encodes a repeat domain of the LRRK2 polypeptide, a Ras-of-complex (Roc) GTPase domain of the LRRK2 polypeptide, a kinase domain of the LRRK2 polypeptide, a WD40 domain of the LRRK2 polypeptide, or a C-terminal of Roc (COR) domain of the LRRK2 polypeptide. In some embodiments, the target RNA encodes the kinase domain of the LRRK2 polypeptide. In some embodiments, the region of the target RNA comprises a mutation as compared to an otherwise comparable region encoding a wildtype polypeptide. In some embodiments, the region of the target RNA comprises a mutation as compared to an otherwise comparable region encoding a wildtype LRRK2 polypeptide. In some embodiments, the mutation comprises a polymorphism. In some embodiments, the mutation is a G to A mutation. In some embodiments, the target RNA comprises at least 80%, 90%, 95%, 97%, 98%, 99%, or 100% sequence identity to any one of SEQ ID NO: 5-SEQ ID NO: 14. In some embodiments, the target RNA encodes a LRRK2 polypeptide comprising at least 80%, 90%, 95%, 97%, 98%, 99%, or 100% sequence identity to any one of SEQ ID NO: 15-SEQ ID NO: 24. In some embodiments, the target RNA encodes a LRRK2 polypeptide comprising a mutation corresponding a G2019S of SEQ ID NO: 15. In some embodiments, the editing of the base is editing of an A corresponding to the 6055th nucleotide in SEQ ID NO: 5. In some embodiments, the target RNA encodes a LRRK2 polypeptide comprising a mutation corresponding to a mutation of Table 3, or any combination of mutations of Table 3. In some embodiments, the engineered polynucleotide comprises at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182. In some embodiments, when the engineered polynucleotide associates with the region of the target RNA, the association comprises hybridized polynucleotide strands. In some embodiments, the hybridized polynucleotide strands at least in part form a double stranded RNA duplex. In some embodiments, the engineered polynucleotide further comprises a chemical modification. In some embodiments, the engineered polynucleotide comprises RNA, DNA, or both. In some embodiments, the engineered polynucleotide comprises the RNA. In some embodiments, the region of the target RNA comprises a translation initiation site.

Also disclosed herein are vectors that comprise an engineered polynucleotide described herein. In some embodiments, the vector is a viral vector. In some embodiments, the viral vector is an AAV vector, and wherein the AAV vector is from an adeno-associated virus having a serotype selected from AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. In some embodiments, the AAV vector is a recombinant AAV (rAAV) vector, a hybrid AAV vector, a chimeric AAV vector, a self-complementary AAV (scAAV) vector, a single-stranded AAV or any combination thereof. In some embodiments, the AAV vector comprises a genome comprising a replication gene and inverted terminal repeats from a first AAV serotype and a capsid protein from a second AAV serotype. In some embodiments, the AAV vector is an AAV 2/5 vector, an AAV 2/6 vector, an AAV 2/7 vector, an AAV2/8 vector, or an AAV 2/9 vector. In some embodiments, the inverted terminal repeats comprise a 5′ inverted terminal repeat, a 3′ inverted terminal repeat, and a mutated inverted terminal repeat. In some embodiments, the mutated inverted terminal repeat lacks a terminal resolution site. In some embodiments, the engineered polynucleotide comprises a targeting sequence that is at least partially complementary to a region of a target RNA, wherein the target RNA: (a) encodes for a Leucine-rich repeat kinase 2 (LRRK2) polypeptide; (b) comprises a non-coding sequence; or (c) comprises (a) and (b), wherein the engineered polynucleotide is configured upon binding to the region of the target RNA, in association with the target RNA, to form a structural feature which recruits an RNA editing entity, wherein the RNA editing entity, when associated with the engineered polynucleotide and the region of the target RNA, facilitates: an editing of a base of a nucleotide in the region of the target RNA, a modulation of translation of the LRRK2 polypeptide, or both. In some embodiments, the targeting sequence is about: 40, 45, 60, 80, 100, 120, 200, or 300 nucleotides in length. In some embodiments, the targeting sequence is about 100 nucleotides in length. In some embodiments, the targeting sequence that is at least partially complementary to the region of the target RNA comprises at least one nucleotide that is not complementary to a nucleotide in the region of the target RNA. In some embodiments, the nucleotide that is not complementary is an adenosine (A) in the region of the target RNA, and wherein the A is comprised in an A/C mismatch. In some embodiments, the nucleotide that is not complementary is an adenosine (A) in the region of the target RNA, and wherein the A is comprised in an internal loop or bulge. In some embodiments, the A is the base of the nucleotide in the region of the target RNA for editing. In some embodiments, the target RNA is selected from the group comprising: an mRNA, a pre-mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA. In some embodiments, the target RNA is an mRNA. In some embodiments, the structural feature comprises: a bulge, a hairpin, an internal loop, and any combination thereof. In some embodiments, the structural feature comprises a bulge. In some embodiments, the bulge is an asymmetric bulge. In some embodiments, the bulge is a symmetric bulge. In some embodiments, the bulge is from 1-4 nucleotides in length. In some embodiments, the structural feature comprises a hairpin. In some embodiments, the structural feature comprises an internal loop. In some embodiments, the internal loop is from 5-50 nucleotides in length. In some embodiments, the internal loop is 6 nucleotides in length. In some embodiments, the engineered polynucleotide comprises at least two internal loops. In some embodiments, the two internal loops are internal symmetrical loops. In some embodiments, the two internal loops are internal symmetrical loops and each side of the two internal loop is 6 nucleotides in length. In some embodiments, the internal loop is an asymmetrical internal loop. In some embodiments, the engineered polynucleotide comprises a structured motif. In some embodiments, the structured motif comprises at least two of: the bulge, the hairpin, and the internal loop. In some embodiments, the structured motif comprises the bulge and the hairpin. In some embodiments, the structured motif comprises the bulge and the internal loop. In some embodiments, the engineered polynucleotide lacks a recruiting domain. In some embodiments, the RNA editing entity comprises an adenosine deaminase acting on RNA (ADAR) polypeptide or biologically active fragment thereof or adenosine deaminases acting on tRNA (ADAT) polypeptide or biologically active fragment thereof. In some embodiments, the ADAR polypeptide or biologically active fragment thereof comprises ADAR1 or ADAR2. In some embodiments, the engineered polynucleotide further comprises an RNA editing entity recruiting domain that is capable of recruiting the RNA editing entity. In some embodiments, the RNA editing entity recruiting domain is at least 1 to about 75 nucleotides in length. In some embodiments, the RNA editing entity recruiting domain is at least 30-50 nucleotides in length. In some embodiments, the RNA editing entity recruiting domain comprises a glutamate ionotropic receptor AMPA type subunit 2 (GluR2) sequence. In some embodiments, the GluR2 sequence comprises at least about 80%, 85%, 90%, 95%, or 99% sequence identity to SEQ ID NO: 1. In some embodiments, the GluR2 sequence comprises SEQ ID NO: 1. In some embodiments, the region is from 5 to 600 nucleotides in length of the target RNA, 40 to 400 nucleotides in length, or 80 to 120 nucleotides in length. In some embodiments, the region is from 50 to 200 nucleotides in length of the target RNA. In some embodiments, the region is about 100 nucleotides in length of the target RNA. In some embodiments, the region of the target RNA comprises at least 60%, 70%, 80%, 85%, 90%, 95%, 97%, 99%, or 100% sequence identity to SEQ ID NO: 73 or SEQ ID NO: 74. In some embodiments, the non-coding sequence comprises a three prime untranslated region (3′ UTR). In some embodiments, the non-coding sequence comprises a five prime untranslated region (5′ UTR). In some embodiments, the editing of the base in the 5′UTR of the region of the target RNA results in at least partially regulating gene translation of the LRRK2 polypeptide. In some embodiments, the editing of the base in the 5′UTR of the region of the target RNA results in facilitating regulation mRNA translation of: the LRRK2 polypeptide. In some embodiments, the target RNA encodes the LRRK2 polypeptide. In some embodiments, the target RNA that encodes the LRRK2 polypeptide comprises at least a portion of: a poly(A) tail, a microRNA response element (MRE), AU-rich element (ARE), hnRNP binding sites or any combination thereof. In some embodiments, the engineered polynucleotide is configured to modulate expression of the LRRK2 polypeptide. In some embodiments, the target RNA encodes a repeat domain of the LRRK2 polypeptide, a Ras-of-complex (Roc) GTPase domain of the LRRK2 polypeptide, a kinase domain of the LRRK2 polypeptide, a WD40 domain of the LRRK2 polypeptide, or a C-terminal of Roc (COR) domain of the LRRK2 polypeptide. In some embodiments, the target RNA encodes the kinase domain of the LRRK2 polypeptide. In some embodiments, the region of the target RNA comprises a mutation as compared to an otherwise comparable region encoding a wildtype polypeptide. In some embodiments, the region of the target RNA comprises a mutation as compared to an otherwise comparable region encoding a wildtype LRRK2 polypeptide. In some embodiments, the mutation comprises a polymorphism. In some embodiments, the mutation is a G to A mutation. In some embodiments, the target RNA comprises at least 80%, 90%, 95%, 97%, 98%, 99%, or 100% sequence identity to any one of SEQ ID NO: 5-SEQ ID NO: 14. In some embodiments, the target RNA encodes a LRRK2 polypeptide comprising at least 80%, 90%, 95%, 97%, 98%, 99%, or 100% sequence identity to any one of SEQ ID NO: 15-SEQ ID NO: 24. In some embodiments, the target RNA encodes a LRRK2 polypeptide comprising a mutation corresponding a G2019S of SEQ ID NO: 15. In some embodiments, the editing of the base is editing of an A corresponding to the 6055^{th} nucleotide in SEQ ID NO: 5. In some embodiments, the target RNA encodes a LRRK2 polypeptide comprising a mutation corresponding to a mutation of Table 3, or any combination of mutations of Table 3. In some embodiments, the engineered polynucleotide comprises at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182. In some embodiments, when the engineered polynucleotide associates with the region of the target RNA, the association comprises hybridized polynucleotide strands. In some embodiments, the hybridized polynucleotide strands at least in part form a double stranded RNA duplex. In some embodiments, the engineered polynucleotide further comprises a chemical modification. In some embodiments, the engineered polynucleotide comprises RNA, DNA, or both. In some embodiments, the engineered polynucleotide comprises the RNA. In some embodiments, the region of the target RNA comprises a translation initiation site.

Also disclosed herein are pharmaceutical compositions in unit dose form that comprise: (a) an engineered polynucleotide as described herein; a vector as described herein, or any combination thereof; and (b) a pharmaceutically acceptable excipient, diluent, or carrier. In some embodiments, a vector comprises an engineered polynucleotide described herein. In some embodiments, the vector is a viral vector. In some embodiments, the viral vector is an AAV vector, and wherein the AAV vector is from an adeno-associated virus having a serotype selected from AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. In some embodiments, the AAV vector is a recombinant AAV (rAAV) vector, a hybrid AAV vector, a chimeric AAV vector, a self-complementary AAV (scAAV) vector, a single-stranded AAV or any combination thereof. In some embodiments, the AAV vector comprises a genome comprising a replication gene and inverted terminal repeats from a first AAV serotype and a capsid protein from a second AAV serotype. In some embodiments, the AAV vector is an AAV 2/5 vector, an AAV 2/6 vector, an AAV 2/7 vector, an AAV2/8 vector, or an AAV 2/9 vector. In some embodiments, the inverted terminal repeats comprise a 5′ inverted terminal repeat, a 3′ inverted terminal repeat, and a mutated inverted terminal repeat. In some embodiments, the mutated inverted terminal repeat lacks a terminal resolution site. In some embodiments, the engineered polynucleotide comprises a targeting sequence that is at least partially complementary to a region of a target RNA, wherein the target RNA: (a) encodes for a Leucine-rich repeat kinase 2 (LRRK2) polypeptide; (b) comprises a non-coding sequence; or (c) comprises (a) and (b), wherein the engineered polynucleotide is configured upon binding to the region of the target RNA, in association with the target RNA, to form a structural feature which recruits an RNA editing entity, wherein the RNA editing entity, when associated with the engineered polynucleotide and the region of the target RNA, facilitates: an editing of a base of a nucleotide in the region of the target RNA, a modulation of translation of the LRRK2 polypeptide, or both. In some embodiments, the targeting sequence is about: 40, 45, 60, 80, 100, 120, 200, or 300 nucleotides in length. In some embodiments, the targeting sequence is about 100 nucleotides in length. In some embodiments, the targeting sequence that is at least partially complementary to the region of the target RNA comprises at least one nucleotide that is not complementary to a nucleotide in the region of the target RNA. In some embodiments, the nucleotide that is not complementary is an adenosine (A) in the region of the target RNA, and wherein the A is comprised in an A/C mismatch. In some embodiments, the nucleotide that is not complementary is an adenosine (A) in the region of the target RNA, and wherein the A is comprised in an internal loop or bulge. In some embodiments, the A is the base of the nucleotide in the region of the target RNA for editing. In some embodiments, the target RNA is selected from the group comprising: an mRNA, a pre-mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA. In some embodiments, the target RNA is an mRNA. In some embodiments, the structural feature comprises: a bulge, a hairpin, an internal loop, and any combination thereof. In some embodiments, the structural feature comprises a bulge. In some embodiments, the bulge is an asymmetric bulge. In some embodiments, the bulge is a symmetric bulge. In some embodiments, the bulge is from 1-4 nucleotides in length. In some embodiments, the structural feature comprises a hairpin. In some embodiments, the structural feature comprises an internal loop. In some embodiments, the internal loop is from 5-50 nucleotides in length. In some embodiments, the internal loop is 6 nucleotides in length. In some embodiments, the engineered polynucleotide comprises at least two internal loops. In some embodiments, the two internal loops are internal symmetrical loops. In some embodiments, the two internal loops are internal symmetrical loops and each side of the two internal loop is 6 nucleotides in length. In some embodiments, the internal loop is an asymmetrical internal loop. In some embodiments, the engineered polynucleotide comprises a structured motif. In some embodiments, the structured motif comprises at least two of: the bulge, the hairpin, and the internal loop. In some embodiments, the structured motif comprises the bulge and the hairpin. In some embodiments, the structured motif comprises the bulge and the internal loop. In some embodiments, the engineered polynucleotide lacks a recruiting domain. In some embodiments, the RNA editing entity comprises an adenosine deaminase acting on RNA (ADAR) polypeptide or biologically active fragment thereof or adenosine deaminases acting on tRNA (ADAT) polypeptide or biologically active fragment thereof. In some embodiments, the ADAR polypeptide or biologically active fragment thereof comprises ADAR1 or ADAR2. In some embodiments, the engineered polynucleotide further comprises an RNA editing entity recruiting domain that is capable of recruiting the RNA editing entity. In some embodiments, the RNA editing entity recruiting domain is at least 1 to about 75 nucleotides in length. In some embodiments, the RNA editing entity recruiting domain is at least 30-50 nucleotides in length. In some embodiments, the RNA editing entity recruiting domain comprises a glutamate ionotropic receptor AMPA type subunit 2 (GluR2) sequence. In some embodiments, the GluR2 sequence comprises at least about 80%, 85%, 90%, 95%, or 99% sequence identity to SEQ ID NO: 1. In some embodiments, the GluR2 sequence comprises SEQ ID NO: 1. In some embodiments, the region is from 5 to 600 nucleotides in length of the target RNA, 40 to 400 nucleotides in length, or 80 to 120 nucleotides in length. In some embodiments, the region is from 50 to 200 nucleotides in length of the target RNA. In some embodiments, the region is about 100 nucleotides in length of the target RNA. In some embodiments, the region of the target RNA comprises at least 60%, 70%, 80%, 85%, 90%, 95%, 97%, 99%, or 100% sequence identity to SEQ ID NO: 73 or SEQ ID NO: 74. In some embodiments, the non-coding sequence comprises a three prime untranslated region (3′ UTR). In some embodiments, the non-coding sequence comprises a five prime untranslated region (5′ UTR). In some embodiments, the editing of the base in the 5′UTR of the region of the target RNA results in at least partially regulating gene translation of the LRRK2 polypeptide. In some embodiments, the editing of the base in the 5′UTR of the region of the target RNA results in facilitating regulation mRNA translation of: the LRRK2 polypeptide. In some embodiments, the target RNA encodes the LRRK2 polypeptide. In some embodiments, the target RNA that encodes the LRRK2 polypeptide comprises at least a portion of: a poly(A) tail, a microRNA response element (MRE), AU-rich element (ARE), hnRNP binding sites or any combination thereof. In some embodiments, the engineered polynucleotide is configured to modulate expression of the LRRK2 polypeptide. In some embodiments, the target RNA encodes a repeat domain of the LRRK2 polypeptide, a Ras-of-complex (Roc) GTPase domain of the LRRK2 polypeptide, a kinase domain of the LRRK2 polypeptide, a WD40 domain of the LRRK2 polypeptide, or a C-terminal of Roc (COR) domain of the LRRK2 polypeptide. In some embodiments, the target RNA encodes the kinase domain of the LRRK2 polypeptide. In some embodiments, the region of the target RNA comprises a mutation as compared to an otherwise comparable region encoding a wildtype polypeptide. In some embodiments, the region of the target RNA comprises a mutation as compared to an otherwise comparable region encoding a wildtype LRRK2 polypeptide. In some embodiments, the mutation comprises a polymorphism. In some embodiments, the mutation is a G to A mutation. In some embodiments, the target RNA comprises at least 80%, 90%, 95%, 97%, 98%, 99%, or 100% sequence identity to any one of SEQ ID NO: 5-SEQ ID NO: 14. In some embodiments, the target RNA encodes a LRRK2 polypeptide comprising at least 80%, 90%, 95%, 97%, 98%, 99%, or 100% sequence identity to any one of SEQ ID NO: 15-SEQ ID NO: 24. In some embodiments, the target RNA encodes a LRRK2 polypeptide comprising a mutation corresponding a G2019S of SEQ ID NO: 15. In some embodiments, the editing of the base is editing of an A corresponding to the 6055^{th} nucleotide in SEQ ID NO: 5. In some embodiments, the target RNA encodes a LRRK2 polypeptide comprising a mutation corresponding to a mutation of Table 3, or any combination of mutations of Table 3. In some embodiments, the engineered polynucleotide comprises at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182. In some embodiments, when the engineered polynucleotide associates with the region of the target RNA, the association comprises hybridized polynucleotide strands. In some embodiments, the hybridized polynucleotide strands at least in part form a double stranded RNA duplex. In some embodiments, the engineered polynucleotide further comprises a chemical modification. In some embodiments, the engineered polynucleotide comprises RNA, DNA, or both. In some embodiments, the engineered polynucleotide comprises the RNA. In some embodiments, the region of the target RNA comprises a translation initiation site.

Also disclosed herein are methods of making a pharmaceutical composition comprising admixing an engineered polynucleotide as described herein with a pharmaceutically acceptable excipient, diluent, or carrier. In some embodiments, the engineered polynucleotide comprises a targeting sequence that is at least partially complementary to a region of a target RNA, wherein the target RNA: (a) encodes for a Leucine-rich repeat kinase 2 (LRRK2) polypeptide; (b) comprises a non-coding sequence; or (c) comprises (a) and (b), wherein the engineered polynucleotide is configured upon binding to the region of the target RNA, in association with the target RNA, to form a structural feature which recruits an RNA editing entity, wherein the RNA editing entity, when associated with the engineered polynucleotide and the region of the target RNA, facilitates: an editing of a base of a nucleotide in the region of the target RNA, a modulation of translation of the LRRK2 polypeptide, or both. In some embodiments, the targeting sequence is about: 40, 45, 60, 80, 100, 120, 200, or 300 nucleotides in length. In some embodiments, the targeting sequence is about 100 nucleotides in length. In some embodiments, the targeting sequence that is at least partially complementary to the region of the target RNA comprises at least one nucleotide that is not complementary to a nucleotide in the region of the target RNA. In some embodiments, the nucleotide that is not complementary is an adenosine (A) in the region of the target RNA, and wherein the A is comprised in an A/C mismatch. In some embodiments, the nucleotide that is not complementary is an adenosine (A) in the region of the target RNA, and wherein the A is comprised in an internal loop or bulge. In some embodiments, the A is the base of the nucleotide in the region of the target RNA for editing. In some embodiments, the target RNA is selected from the group comprising: an mRNA, a pre-mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA. In some embodiments, the target RNA is an mRNA. In some embodiments, the structural feature comprises: a bulge, a hairpin, an internal loop, and any combination thereof. In some embodiments, the structural feature comprises a bulge. In some embodiments, the bulge is an asymmetric bulge. In some embodiments, the bulge is a symmetric bulge. In some embodiments, the bulge is from 1-4 nucleotides in length. In some embodiments, the structural feature comprises a hairpin. In some embodiments, the structural feature comprises an internal loop. In some embodiments, the internal loop is from 5-50 nucleotides in length. In some embodiments, the internal loop is 6 nucleotides in length. In some embodiments, the engineered polynucleotide comprises at least two internal loops. In some embodiments, the two internal loops are internal symmetrical loops. In some embodiments, the two internal loops are internal symmetrical loops and each side of the two internal loop is 6 nucleotides in length. In some embodiments, the internal loop is an asymmetrical internal loop. In some embodiments, the engineered polynucleotide comprises a structured motif. In some embodiments, the structured motif comprises at least two of: the bulge, the hairpin, and the internal loop. In some embodiments, the structured motif comprises the bulge and the hairpin. In some embodiments, the structured motif comprises the bulge and the internal loop. In some embodiments, the engineered polynucleotide lacks a recruiting domain. In some embodiments, the RNA editing entity comprises an adenosine deaminase acting on RNA (ADAR) polypeptide or biologically active fragment thereof or adenosine deaminases acting on tRNA (ADAT) polypeptide or biologically active fragment thereof. In some embodiments, the ADAR polypeptide or biologically active fragment thereof comprises ADAR1 or ADAR2. In some embodiments, the engineered polynucleotide further comprises an RNA editing entity recruiting domain that is capable of recruiting the RNA editing entity. In some embodiments, the RNA editing entity recruiting domain is at least 1 to about 75 nucleotides in length. In some embodiments, the RNA editing entity recruiting domain is at least 30-50 nucleotides in length. In some embodiments, the RNA editing entity recruiting domain comprises a glutamate ionotropic receptor AMPA type subunit 2 (GluR2) sequence. In some embodiments, the GluR2 sequence comprises at least about 80%, 85%, 90%, 95%, or 99% sequence identity to SEQ ID NO: 1. In some embodiments, the GluR2 sequence comprises SEQ ID NO: 1. In some embodiments, the region is from 5 to 600 nucleotides in length of the target RNA, 40 to 400 nucleotides in length, or 80 to 120 nucleotides in length. In some embodiments, the region is from 50 to 200 nucleotides in length of the target RNA. In some embodiments, the region is about 100 nucleotides in length of the target RNA. In some embodiments, the region of the target RNA comprises at least 60%, 70%, 80%, 85%, 90%, 95%, 97%, 99%, or 100% sequence identity to SEQ ID NO: 73 or SEQ ID NO: 74. In some embodiments, the non-coding sequence comprises a three prime untranslated region (3′ UTR). In some embodiments, the non-coding sequence comprises a five prime untranslated region (5′ UTR). In some embodiments, the editing of the base in the 5′UTR of the region of the target RNA results in at least partially regulating gene translation of the LRRK2 polypeptide. In some embodiments, the editing of the base in the 5′UTR of the region of the target RNA results in facilitating regulation mRNA translation of: the LRRK2 polypeptide. In some embodiments, the target RNA encodes the LRRK2 polypeptide. In some embodiments, the target RNA that encodes the LRRK2 polypeptide comprises at least a portion of: a poly(A) tail, a microRNA response element (MRE), AU-rich element (ARE), hnRNP binding sites or any combination thereof. In some embodiments, the engineered polynucleotide is configured to modulate expression of the LRRK2 polypeptide. In some embodiments, the target RNA encodes a repeat domain of the LRRK2 polypeptide, a Ras-of-complex (Roc) GTPase domain of the LRRK2 polypeptide, a kinase domain of the LRRK2 polypeptide, a WD40 domain of the LRRK2 polypeptide, or a C-terminal of Roc (COR) domain of the LRRK2 polypeptide. In some embodiments, the target RNA encodes the kinase domain of the LRRK2 polypeptide. In some embodiments, the region of the target RNA comprises a mutation as compared to an otherwise comparable region encoding a wildtype polypeptide. In some embodiments, the region of the target RNA comprises a mutation as compared to an otherwise comparable region encoding a wildtype LRRK2 polypeptide. In some embodiments, the mutation comprises a polymorphism. In some embodiments, the mutation is a G to A mutation. In some embodiments, the target RNA comprises at least 80%, 90%, 95%, 97%, 98%, 99%, or 100% sequence identity to any one of SEQ ID NO: 5-SEQ ID NO: 14. In some embodiments, the target RNA encodes a LRRK2 polypeptide comprising at least 80%, 90%, 95%, 97%, 98%, 99%, or 100% sequence identity to any one of SEQ ID NO: 15-SEQ ID NO: 24. In some embodiments, the target RNA encodes a LRRK2 polypeptide comprising a mutation corresponding a G2019S of SEQ ID NO: 15. In some embodiments, the editing of the base is editing of an A corresponding to the 6055^{th} nucleotide in SEQ ID NO: 5. In some embodiments, the target RNA encodes a LRRK2 polypeptide comprising a mutation corresponding to a mutation of Table 3, or any combination of mutations of Table 3. In some embodiments, the engineered polynucleotide comprises at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182. In some embodiments, when the engineered polynucleotide associates with the region of the target RNA, the association comprises hybridized polynucleotide strands. In some embodiments, the hybridized polynucleotide strands at least in part form a double stranded RNA duplex. In some embodiments, the engineered polynucleotide further comprises a chemical modification. In some embodiments, the engineered polynucleotide comprises RNA, DNA, or both. In some embodiments, the engineered polynucleotide comprises the RNA. In some embodiments, the region of the target RNA comprises a translation initiation site.

Also disclosed herein are isolated cells comprising an engineered polynucleotide as described herein, a vector as described herein, or both. In some embodiments, a vector comprises an engineered polynucleotide described herein. In some embodiments, the vector is a viral vector. In some embodiments, the viral vector is an AAV vector, and wherein the AAV vector is from an adeno-associated virus having a serotype selected from AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. In some embodiments, the AAV vector is a recombinant AAV (rAAV) vector, a hybrid AAV vector, a chimeric AAV vector, a self-complementary AAV (scAAV) vector, a single-stranded AAV or any combination thereof. In some embodiments, the AAV vector comprises a genome comprising a replication gene and inverted terminal repeats from a first AAV serotype and a capsid protein from a second AAV serotype. In some embodiments, the AAV vector is an AAV 2/5 vector, an AAV 2/6 vector, an AAV 2/7 vector, an AAV2/8 vector, or an AAV 2/9 vector. In some embodiments, the inverted terminal repeats comprise a 5′ inverted terminal repeat, a 3′ inverted terminal repeat, and a mutated inverted terminal repeat. In some embodiments, the mutated inverted terminal repeat lacks a terminal resolution site. In some embodiments, the engineered polynucleotide comprises a targeting sequence that is at least partially complementary to a region of a target RNA, wherein the target RNA: (a) encodes for a Leucine-rich repeat kinase 2 (LRRK2) polypeptide; (b) comprises a non-coding sequence; or (c) comprises (a) and (b), wherein the engineered polynucleotide is configured upon binding to the region of the target RNA, in association with the target RNA, to form a structural feature which recruits an RNA editing entity, wherein the RNA editing entity, when associated with the engineered polynucleotide and the region of the target RNA, facilitates: an editing of a base of a nucleotide in the region of the target RNA, a modulation of translation of the LRRK2 polypeptide, or both. In some embodiments, the targeting sequence is about: 40, 45, 60, 80, 100, 120, 200, or 300 nucleotides in length. In some embodiments, the targeting sequence is about 100 nucleotides in length. In some embodiments, the targeting sequence that is at least partially complementary to the region of the target RNA comprises at least one nucleotide that is not complementary to a nucleotide in the region of the target RNA. In some embodiments, the nucleotide that is not complementary is an adenosine (A) in the region of the target RNA, and wherein the A is comprised in an A/C mismatch. In some embodiments, the nucleotide that is not complementary is an adenosine (A) in the region of the target RNA, and wherein the A is comprised in an internal loop or bulge. In some embodiments, the A is the base of the nucleotide in the region of the target RNA for editing. In some embodiments, the target RNA is selected from the group comprising: an mRNA, a pre-mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA. In some embodiments, the target RNA is an mRNA. In some embodiments, the structural feature comprises: a bulge, a hairpin, an internal loop, and any combination thereof. In some embodiments, the structural feature comprises a bulge. In some embodiments, the bulge is an asymmetric bulge. In some embodiments, the bulge is a symmetric bulge. In some embodiments, the bulge is from 1-4 nucleotides in length. In some embodiments, the structural feature comprises a hairpin. In some embodiments, the structural feature comprises an internal loop. In some embodiments, the internal loop is from 5-50 nucleotides in length. In some embodiments, the internal loop is 6 nucleotides in length. In some embodiments, the engineered polynucleotide comprises at least two internal loops. In some embodiments, the two internal loops are internal symmetrical loops. In some embodiments, the two internal loops are internal symmetrical loops and each side of the two internal loop is 6 nucleotides in length. In some embodiments, the internal loop is an asymmetrical internal loop. In some embodiments, the engineered polynucleotide comprises a structured motif. In some embodiments, the structured motif comprises at least two of: the bulge, the hairpin, and the internal loop. In some embodiments, the structured motif comprises the bulge and the hairpin. In some embodiments, the structured motif comprises the bulge and the internal loop. In some embodiments, the engineered polynucleotide lacks a recruiting domain. In some embodiments, the RNA editing entity comprises an adenosine deaminase acting on RNA (ADAR) polypeptide or biologically active fragment thereof or adenosine deaminases acting on tRNA (ADAT) polypeptide or biologically active fragment thereof. In some embodiments, the ADAR polypeptide or biologically active fragment thereof comprises ADAR1 or ADAR2. In some embodiments, the engineered polynucleotide further comprises an RNA editing entity recruiting domain that is capable of recruiting the RNA editing entity. In some embodiments, the RNA editing entity recruiting domain is at least 1 to about 75 nucleotides in length. In some embodiments, the RNA editing entity recruiting domain is at least 30-50 nucleotides in length. In some embodiments, the RNA editing entity recruiting domain comprises a glutamate ionotropic receptor AMPA type subunit 2 (GluR2) sequence. In some embodiments, the GluR2 sequence comprises at least about 80%, 85%, 90%, 95%, or 99% sequence identity to SEQ ID NO: 1. In some embodiments, the GluR2 sequence comprises SEQ ID NO: 1. In some embodiments, the region is from 5 to 600 nucleotides in length of the target RNA, 40 to 400 nucleotides in length, or 80 to 120 nucleotides in length. In some embodiments, the region is from 50 to 200 nucleotides in length of the target RNA. In some embodiments, the region is about 100 nucleotides in length of the target RNA. In some embodiments, the region of the target RNA comprises at least 60%, 70%, 80%, 85%, 90%, 95%, 97%, 99%, or 100% sequence identity to SEQ ID NO: 73 or SEQ ID NO: 74. In some embodiments, the non-coding sequence comprises a three prime untranslated region (3′ UTR). In some embodiments, the non-coding sequence comprises a five prime untranslated region (5′ UTR). In some embodiments, the editing of the base in the 5′UTR of the region of the target RNA results in at least partially regulating gene translation of the LRRK2 polypeptide. In some embodiments, the editing of the base in the 5′UTR of the region of the target RNA results in facilitating regulation mRNA translation of: the LRRK2 polypeptide. In some embodiments, the target RNA encodes the LRRK2 polypeptide. In some embodiments, the target RNA that encodes the LRRK2 polypeptide comprises at least a portion of: a poly(A) tail, a microRNA response element (MRE), AU-rich element (ARE), hnRNP binding sites or any combination thereof. In some embodiments, the engineered polynucleotide is configured to modulate expression of the LRRK2 polypeptide. In some embodiments, the target RNA encodes a repeat domain of the LRRK2 polypeptide, a Ras-of-complex (Roc) GTPase domain of the LRRK2 polypeptide, a kinase domain of the LRRK2 polypeptide, a WD40 domain of the LRRK2 polypeptide, or a C-terminal of Roc (COR) domain of the LRRK2 polypeptide. In some embodiments, the target RNA encodes the kinase domain of the LRRK2 polypeptide. In some embodiments, the region of the target RNA comprises a mutation as compared to an otherwise comparable region encoding a wildtype polypeptide. In some embodiments, the region of the target RNA comprises a mutation as compared to an otherwise comparable region encoding a wildtype LRRK2 polypeptide. In some embodiments, the mutation comprises a polymorphism. In some embodiments, the mutation is a G to A mutation. In some embodiments, the target RNA comprises at least 80%, 90%, 95%, 97%, 98%, 99%, or 100% sequence identity to any one of SEQ ID NO: 5-SEQ ID NO: 14. In some embodiments, the target RNA encodes a LRRK2 polypeptide comprising at least 80%, 90%, 95%, 97%, 98%, 99%, or 100% sequence identity to any one of SEQ ID NO: 15-SEQ ID NO: 24. In some embodiments, the target RNA encodes a LRRK2 polypeptide comprising a mutation corresponding a G2019S of SEQ ID NO: 15. In some embodiments, the editing of the base is editing of an A corresponding to the 6055^{th} nucleotide in SEQ ID NO: 5. In some embodiments, the target RNA encodes a LRRK2 polypeptide comprising a mutation corresponding to a mutation of Table 3, or any combination of mutations of Table 3. In some embodiments, the engineered polynucleotide comprises at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182. In some embodiments, when the engineered polynucleotide associates with the region of the target RNA, the association comprises hybridized polynucleotide strands. In some embodiments, the hybridized polynucleotide strands at least in part form a double stranded RNA duplex. In some embodiments, the engineered polynucleotide further comprises a chemical modification. In some embodiments, the engineered polynucleotide comprises RNA, DNA, or both. In some embodiments, the engineered polynucleotide comprises the RNA. In some embodiments, the region of the target RNA comprises a translation initiation site.

Also disclosed herein are kits comprising an engineered polynucleotide as described herein, a vector as described herein, or both in a container. In some embodiments, a vector comprises an engineered polynucleotide described herein. In some embodiments, the vector is a viral vector. In some embodiments, the viral vector is an AAV vector, and wherein the AAV vector is from an adeno-associated virus having a serotype selected from AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. In some embodiments, the AAV vector is a recombinant AAV (rAAV) vector, a hybrid AAV vector, a chimeric AAV vector, a self-complementary AAV (scAAV) vector, a single-stranded AAV or any combination thereof. In some embodiments, the AAV vector comprises a genome comprising a replication gene and inverted terminal repeats from a first AAV serotype and a capsid protein from a second AAV serotype. In some embodiments, the AAV vector is an AAV 2/5 vector, an AAV 2/6 vector, an AAV 2/7 vector, an AAV2/8 vector, or an AAV 2/9 vector. In some embodiments, the inverted terminal repeats comprise a 5′ inverted terminal repeat, a 3′ inverted terminal repeat, and a mutated inverted terminal repeat. In some embodiments, the mutated inverted terminal repeat lacks a terminal resolution site. In some embodiments, the engineered polynucleotide comprises a targeting sequence that is at least partially complementary to a region of a target RNA, wherein the target RNA: (a) encodes for a Leucine-rich repeat kinase 2 (LRRK2) polypeptide; (b) comprises a non-coding sequence; or (c) comprises (a) and (b), wherein the engineered polynucleotide is configured upon binding to the region of the target RNA, in association with the target RNA, to form a structural feature which recruits an RNA editing entity, wherein the RNA editing entity, when associated with the engineered polynucleotide and the region of the target RNA, facilitates: an editing of a base of a nucleotide in the region of the target RNA, a modulation of translation of the LRRK2 polypeptide, or both. In some embodiments, the targeting sequence is about: 40, 45, 60, 80, 100, 120, 200, or 300 nucleotides in length. In some embodiments, the targeting sequence is about 100 nucleotides in length. In some embodiments, the targeting sequence that is at least partially complementary to the region of the target RNA comprises at least one nucleotide that is not complementary to a nucleotide in the region of the target RNA. In some embodiments, the nucleotide that is not complementary is an adenosine (A) in the region of the target RNA, and wherein the A is comprised in an A/C mismatch. In some embodiments, the nucleotide that is not complementary is an adenosine (A) in the region of the target RNA, and wherein the A is comprised in an internal loop or bulge. In some embodiments, the A is the base of the nucleotide in the region of the target RNA for editing. In some embodiments, the target RNA is selected from the group comprising: an mRNA, a pre-mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA. In some embodiments, the target RNA is an mRNA. In some embodiments, the structural feature comprises: a bulge, a hairpin, an internal loop, and any combination thereof. In some embodiments, the structural feature comprises a bulge. In some embodiments, the bulge is an asymmetric bulge. In some embodiments, the bulge is a symmetric bulge. In some embodiments, the bulge is from 1-4 nucleotides in length. In some embodiments, the structural feature comprises a hairpin. In some embodiments, the structural feature comprises an internal loop. In some embodiments, the internal loop is from 5-50 nucleotides in length. In some embodiments, the internal loop is 6 nucleotides in length. In some embodiments, the engineered polynucleotide comprises at least two internal loops. In some embodiments, the two internal loops are internal symmetrical loops. In some embodiments, the two internal loops are internal symmetrical loops and each side of the two internal loop is 6 nucleotides in length. In some embodiments, the internal loop is an asymmetrical internal loop. In some embodiments, the engineered polynucleotide comprises a structured motif. In some embodiments, the structured motif comprises at least two of: the bulge, the hairpin, and the internal loop. In some embodiments, the structured motif comprises the bulge and the hairpin. In some embodiments, the structured motif comprises the bulge and the internal loop. In some embodiments, the engineered polynucleotide lacks a recruiting domain. In some embodiments, the RNA editing entity comprises an adenosine deaminase acting on RNA (ADAR) polypeptide or biologically active fragment thereof or adenosine deaminases acting on tRNA (ADAT) polypeptide or biologically active fragment thereof. In some embodiments, the ADAR polypeptide or biologically active fragment thereof comprises ADAR1 or ADAR2. In some embodiments, the engineered polynucleotide further comprises an RNA editing entity recruiting domain that is capable of recruiting the RNA editing entity. In some embodiments, the RNA editing entity recruiting domain is at least 1 to about 75 nucleotides in length. In some embodiments, the RNA editing entity recruiting domain is at least 30-50 nucleotides in length. In some embodiments, the RNA editing entity recruiting domain comprises a glutamate ionotropic receptor AMPA type subunit 2 (GluR2) sequence. In some embodiments, the GluR2 sequence comprises at least about 80%, 85%, 90%, 95%, or 99% sequence identity to SEQ ID NO: 1. In some embodiments, the GluR2 sequence comprises SEQ ID NO: 1. In some embodiments, the region is from 5 to 600 nucleotides in length of the target RNA, 40 to 400 nucleotides in length, or 80 to 120 nucleotides in length. In some embodiments, the region is from 50 to 200 nucleotides in length of the target RNA. In some embodiments, the region is about 100 nucleotides in length of the target RNA. In some embodiments, the region of the target RNA comprises at least 60%, 70%, 80%, 85%, 90%, 95%, 97%, 99%, or 100% sequence identity to SEQ ID NO: 73 or SEQ ID NO: 74. In some embodiments, the non-coding sequence comprises a three prime untranslated region (3′ UTR). In some embodiments, the non-coding sequence comprises a five prime untranslated region (5′ UTR). In some embodiments, the editing of the base in the 5′UTR of the region of the target RNA results in at least partially regulating gene translation of the LRRK2 polypeptide. In some embodiments, the editing of the base in the 5′UTR of the region of the target RNA results in facilitating regulation mRNA translation of: the LRRK2 polypeptide. In some embodiments, the target RNA encodes the LRRK2 polypeptide. In some embodiments, the target RNA that encodes the LRRK2 polypeptide comprises at least a portion of: a poly(A) tail, a microRNA response element (MRE), AU-rich element (ARE), hnRNP binding sites or any combination thereof. In some embodiments, the engineered polynucleotide is configured to modulate expression of the LRRK2 polypeptide. In some embodiments, the target RNA encodes a repeat domain of the LRRK2 polypeptide, a Ras-of-complex (Roc) GTPase domain of the LRRK2 polypeptide, a kinase domain of the LRRK2 polypeptide, a WD40 domain of the LRRK2 polypeptide, or a C-terminal of Roc (COR) domain of the LRRK2 polypeptide. In some embodiments, the target RNA encodes the kinase domain of the LRRK2 polypeptide. In some embodiments, the region of the target RNA comprises a mutation as compared to an otherwise comparable region encoding a wildtype polypeptide. In some embodiments, the region of the target RNA comprises a mutation as compared to an otherwise comparable region encoding a wildtype LRRK2 polypeptide. In some embodiments, the mutation comprises a polymorphism. In some embodiments, the mutation is a G to A mutation. In some embodiments, the target RNA comprises at least 80%, 90%, 95%, 97%, 98%, 99%, or 100% sequence identity to any one of SEQ ID NO: 5-SEQ ID NO: 14. In some embodiments, the target RNA encodes a LRRK2 polypeptide comprising at least 80%, 90%, 95%, 97%, 98%, 99%, or 100% sequence identity to any one of SEQ ID NO: 15-SEQ ID NO: 24. In some embodiments, the target RNA encodes a LRRK2 polypeptide comprising a mutation corresponding a G2019S of SEQ ID NO: 15. In some embodiments, the editing of the base is editing of an A corresponding to the 6055^{th} nucleotide in SEQ ID NO: 5. In some embodiments, the target RNA encodes a LRRK2 polypeptide comprising a mutation corresponding to a mutation of Table 3, or any combination of mutations of Table 3. In some embodiments, the engineered polynucleotide comprises at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182. In some embodiments, when the engineered polynucleotide associates with the region of the target RNA, the association comprises hybridized polynucleotide strands. In some embodiments, the hybridized polynucleotide strands at least in part form a double stranded RNA duplex. In some embodiments, the engineered polynucleotide further comprises a chemical modification. In some embodiments, the engineered polynucleotide comprises RNA, DNA, or both. In some embodiments, the engineered polynucleotide comprises the RNA. In some embodiments, the region of the target RNA comprises a translation initiation site.

Also disclosed herein are methods of making a kit comprising inserting an engineered polynucleotide as described herein, a vector as described herein, or both in a container. In some embodiments, a vector comprises an engineered polynucleotide described herein. In some embodiments, the vector is a viral vector. In some embodiments, the viral vector is an AAV vector, and wherein the AAV vector is from an adeno-associated virus having a serotype selected from AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. In some embodiments, the AAV vector is a recombinant AAV (rAAV) vector, a hybrid AAV vector, a chimeric AAV vector, a self-complementary AAV (scAAV) vector, a single-stranded AAV or any combination thereof. In some embodiments, the AAV vector comprises a genome comprising a replication gene and inverted terminal repeats from a first AAV serotype and a capsid protein from a second AAV serotype. In some embodiments, the AAV vector is an AAV 2/5 vector, an AAV 2/6 vector, an AAV 2/7 vector, an AAV2/8 vector, or an AAV 2/9 vector. In some embodiments, the inverted terminal repeats comprise a 5′ inverted terminal repeat, a 3′ inverted terminal repeat, and a mutated inverted terminal repeat. In some embodiments, the mutated inverted terminal repeat lacks a terminal resolution site. In some embodiments, the engineered polynucleotide comprises a targeting sequence that is at least partially complementary to a region of a target RNA, wherein the target RNA: (a) encodes for a Leucine-rich repeat kinase 2 (LRRK2) polypeptide; (b) comprises a non-coding sequence; or (c) comprises (a) and (b), wherein the engineered polynucleotide is configured upon binding to the region of the target RNA, in association with the target RNA, to form a structural feature which recruits an RNA editing entity, wherein the RNA editing entity, when associated with the engineered polynucleotide and the region of the target RNA, facilitates: an editing of a base of a nucleotide in the region of the target RNA, a modulation of translation of the LRRK2 polypeptide, or both. In some embodiments, the targeting sequence is about: 40, 45, 60, 80, 100, 120, 200, or 300 nucleotides in length. In some embodiments, the targeting sequence is about 100 nucleotides in length. In some embodiments, the targeting sequence that is at least partially complementary to the region of the target RNA comprises at least one nucleotide that is not complementary to a nucleotide in the region of the target RNA. In some embodiments, the nucleotide that is not complementary is an adenosine (A) in the region of the target RNA, and wherein the A is comprised in an A/C mismatch. In some embodiments, the nucleotide that is not complementary is an adenosine (A) in the region of the target RNA, and wherein the A is comprised in an internal loop or bulge. In some embodiments, the A is the base of the nucleotide in the region of the target RNA for editing. In some embodiments, the target RNA is selected from the group comprising: an mRNA, a pre-mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA. In some embodiments, the target RNA is an mRNA. In some embodiments, the structural feature comprises: a bulge, a hairpin, an internal loop, and any combination thereof. In some embodiments, the structural feature comprises a bulge. In some embodiments, the bulge is an asymmetric bulge. In some embodiments, the bulge is a symmetric bulge. In some embodiments, the bulge is from 1-4 nucleotides in length. In some embodiments, the structural feature comprises a hairpin. In some embodiments, the structural feature comprises an internal loop. In some embodiments, the internal loop is from 5-50 nucleotides in length. In some embodiments, the internal loop is 6 nucleotides in length. In some embodiments, the engineered polynucleotide comprises at least two internal loops. In some embodiments, the two internal loops are internal symmetrical loops. In some embodiments, the two internal loops are internal symmetrical loops and each side of the two internal loop is 6 nucleotides in length. In some embodiments, the internal loop is an asymmetrical internal loop. In some embodiments, the engineered polynucleotide comprises a structured motif. In some embodiments, the structured motif comprises at least two of: the bulge, the hairpin, and the internal loop. In some embodiments, the structured motif comprises the bulge and the hairpin. In some embodiments, the structured motif comprises the bulge and the internal loop. In some embodiments, the engineered polynucleotide lacks a recruiting domain. In some embodiments, the RNA editing entity comprises an adenosine deaminase acting on RNA (ADAR) polypeptide or biologically active fragment thereof or adenosine deaminases acting on tRNA (ADAT) polypeptide or biologically active fragment thereof. In some embodiments, the ADAR polypeptide or biologically active fragment thereof comprises ADAR1 or ADAR2. In some embodiments, the engineered polynucleotide further comprises an RNA editing entity recruiting domain that is capable of recruiting the RNA editing entity. In some embodiments, the RNA editing entity recruiting domain is at least 1 to about 75 nucleotides in length. In some embodiments, the RNA editing entity recruiting domain is at least 30-50 nucleotides in length. In some embodiments, the RNA editing entity recruiting domain comprises a glutamate ionotropic receptor AMPA type subunit 2 (GluR2) sequence. In some embodiments, the GluR2 sequence comprises at least about 80%, 85%, 90%, 95%, or 99% sequence identity to SEQ ID NO: 1. In some embodiments, the GluR2 sequence comprises SEQ ID NO: 1. In some embodiments, the region is from 5 to 600 nucleotides in length of the target RNA, 40 to 400 nucleotides in length, or 80 to 120 nucleotides in length. In some embodiments, the region is from 50 to 200 nucleotides in length of the target RNA. In some embodiments, the region is about 100 nucleotides in length of the target RNA. In some embodiments, the region of the target RNA comprises at least 60%, 70%, 80%, 85%, 90%, 95%, 97%, 99%, or 100% sequence identity to SEQ ID NO: 73 or SEQ ID NO: 74. In some embodiments, the non-coding sequence comprises a three prime untranslated region (3′ UTR). In some embodiments, the non-coding sequence comprises a five prime untranslated region (5′ UTR). In some embodiments, the editing of the base in the 5′UTR of the region of the target RNA results in at least partially regulating gene translation of the LRRK2 polypeptide. In some embodiments, the editing of the base in the 5′UTR of the region of the target RNA results in facilitating regulation mRNA translation of: the LRRK2 polypeptide. In some embodiments, the target RNA encodes the LRRK2 polypeptide. In some embodiments, the target RNA that encodes the LRRK2 polypeptide comprises at least a portion of: a poly(A) tail, a microRNA response element (MRE), AU-rich element (ARE), hnRNP binding sites or any combination thereof. In some embodiments, the engineered polynucleotide is configured to modulate expression of the LRRK2 polypeptide. In some embodiments, the target RNA encodes a repeat domain of the LRRK2 polypeptide, a Ras-of-complex (Roc) GTPase domain of the LRRK2 polypeptide, a kinase domain of the LRRK2 polypeptide, a WD40 domain of the LRRK2 polypeptide, or a C-terminal of Roc (COR) domain of the LRRK2 polypeptide. In some embodiments, the target RNA encodes the kinase domain of the LRRK2 polypeptide. In some embodiments, the region of the target RNA comprises a mutation as compared to an otherwise comparable region encoding a wildtype polypeptide. In some embodiments, the region of the target RNA comprises a mutation as compared to an otherwise comparable region encoding a wildtype LRRK2 polypeptide. In some embodiments, the mutation comprises a polymorphism. In some embodiments, the mutation is a G to A mutation. In some embodiments, the target RNA comprises at least 80%, 90%, 95%, 97%, 98%, 99%, or 100% sequence identity to any one of SEQ ID NO: 5-SEQ ID NO: 14. In some embodiments, the target RNA encodes a LRRK2 polypeptide comprising at least 80%, 90%, 95%, 97%, 98%, 99%, or 100% sequence identity to any one of SEQ ID NO: 15-SEQ ID NO: 24. In some embodiments, the target RNA encodes a LRRK2 polypeptide comprising a mutation corresponding a G2019S of SEQ ID NO: 15. In some embodiments, the editing of the base is editing of an A corresponding to the 6055^{th} nucleotide in SEQ ID NO: 5. In some embodiments, the target RNA encodes a LRRK2 polypeptide comprising a mutation corresponding to a mutation of Table 3, or any combination of mutations of Table 3. In some embodiments, the engineered polynucleotide comprises at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182. In some embodiments, when the engineered polynucleotide associates with the region of the target RNA, the association comprises hybridized polynucleotide strands. In some embodiments, the hybridized polynucleotide strands at least in part form a double stranded RNA duplex. In some embodiments, the engineered polynucleotide further comprises a chemical modification. In some embodiments, the engineered polynucleotide comprises RNA, DNA, or both. In some embodiments, the engineered polynucleotide comprises the RNA. In some embodiments, the region of the target RNA comprises a translation initiation site.

Also disclosed herein are methods of treating or preventing a disease or condition in a subject in need thereof, the method comprising administering to a subject in need thereof: (a) a vector as described herein; (b) a pharmaceutical composition as described herein; or (c) (a) and (b). In some embodiments, a pharmaceutical composition is in unit dose form and comprises: (a) an engineered polynucleotide as described herein; a vector as described herein, or any combination thereof; and (b) a pharmaceutically acceptable excipient, diluent, or carrier. In some embodiments, a vector comprises an engineered polynucleotide described herein. In some embodiments, the vector is a viral vector. In some embodiments, the viral vector is an AAV vector, and wherein the AAV vector is from an adeno-associated virus having a serotype selected from AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. In some embodiments, the AAV vector is a recombinant AAV (rAAV) vector, a hybrid AAV vector, a chimeric AAV vector, a self-complementary AAV (scAAV) vector, a single-stranded AAV or any combination thereof. In some embodiments, the AAV vector comprises a genome comprising a replication gene and inverted terminal repeats from a first AAV serotype and a capsid protein from a second AAV serotype. In some embodiments, the AAV vector is an AAV 2/5 vector, an AAV 2/6 vector, an AAV 2/7 vector, an AAV2/8 vector, or an AAV 2/9 vector. In some embodiments, the inverted terminal repeats comprise a 5′ inverted terminal repeat, a 3′ inverted terminal repeat, and a mutated inverted terminal repeat. In some embodiments, the mutated inverted terminal repeat lacks a terminal resolution site. In some embodiments, the engineered polynucleotide comprises a targeting sequence that is at least partially complementary to a region of a target RNA, wherein the target RNA: (a) encodes for a Leucine-rich repeat kinase 2 (LRRK2) polypeptide; (b) comprises a non-coding sequence; or (c) comprises (a) and (b), wherein the engineered polynucleotide is configured upon binding to the region of the target RNA, in association with the target RNA, to form a structural feature which recruits an RNA editing entity, wherein the RNA editing entity, when associated with the engineered polynucleotide and the region of the target RNA, facilitates: an editing of a base of a nucleotide in the region of the target RNA, a modulation of translation of the LRRK2 polypeptide, or both. In some embodiments, the targeting sequence is about: 40, 45, 60, 80, 100, 120, 200, or 300 nucleotides in length. In some embodiments, the targeting sequence is about 100 nucleotides in length. In some embodiments, the targeting sequence that is at least partially complementary to the region of the target RNA comprises at least one nucleotide that is not complementary to a nucleotide in the region of the target RNA. In some embodiments, the nucleotide that is not complementary is an adenosine (A) in the region of the target RNA, and wherein the A is comprised in an A/C mismatch. In some embodiments, the nucleotide that is not complementary is an adenosine (A) in the region of the target RNA, and wherein the A is comprised in an internal loop or bulge. In some embodiments, the A is the base of the nucleotide in the region of the target RNA for editing. In some embodiments, the target RNA is selected from the group comprising: an mRNA, a pre-mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA. In some embodiments, the target RNA is an mRNA. In some embodiments, the structural feature comprises: a bulge, a hairpin, an internal loop, and any combination thereof. In some embodiments, the structural feature comprises a bulge. In some embodiments, the bulge is an asymmetric bulge. In some embodiments, the bulge is a symmetric bulge. In some embodiments, the bulge is from 1-4 nucleotides in length. In some embodiments, the structural feature comprises a hairpin. In some embodiments, the structural feature comprises an internal loop. In some embodiments, the internal loop is from 5-50 nucleotides in length. In some embodiments, the internal loop is 6 nucleotides in length. In some embodiments, the engineered polynucleotide comprises at least two internal loops. In some embodiments, the two internal loops are internal symmetrical loops. In some embodiments, the two internal loops are internal symmetrical loops and each side of the two internal loop is 6 nucleotides in length. In some embodiments, the internal loop is an asymmetrical internal loop. In some embodiments, the engineered polynucleotide comprises a structured motif. In some embodiments, the structured motif comprises at least two of: the bulge, the hairpin, and the internal loop. In some embodiments, the structured motif comprises the bulge and the hairpin. In some embodiments, the structured motif comprises the bulge and the internal loop. In some embodiments, the engineered polynucleotide lacks a recruiting domain. In some embodiments, the RNA editing entity comprises an adenosine deaminase acting on RNA (ADAR) polypeptide or biologically active fragment thereof or adenosine deaminases acting on tRNA (ADAT) polypeptide or biologically active fragment thereof. In some embodiments, the ADAR polypeptide or biologically active fragment thereof comprises ADAR1 or ADAR2. In some embodiments, the engineered polynucleotide further comprises an RNA editing entity recruiting domain that is capable of recruiting the RNA editing entity. In some embodiments, the RNA editing entity recruiting domain is at least 1 to about 75 nucleotides in length. In some embodiments, the RNA editing entity recruiting domain is at least 30-50 nucleotides in length. In some embodiments, the RNA editing entity recruiting domain comprises a glutamate ionotropic receptor AMPA type subunit 2 (GluR2) sequence. In some embodiments, the GluR2 sequence comprises at least about 80%, 85%, 90%, 95%, or 99% sequence identity to SEQ ID NO: 1. In some embodiments, the GluR2 sequence comprises SEQ ID NO: 1. In some embodiments, the region is from 5 to 600 nucleotides in length of the target RNA, 40 to 400 nucleotides in length, or 80 to 120 nucleotides in length. In some embodiments, the region is from 50 to 200 nucleotides in length of the target RNA. In some embodiments, the region is about 100 nucleotides in length of the target RNA. In some embodiments, the region of the target RNA comprises at least 60%, 70%, 80%, 85%, 90%, 95%, 97%, 99%, or 100% sequence identity to SEQ ID NO: 73 or SEQ ID NO: 74. In some embodiments, the non-coding sequence comprises a three prime untranslated region (3′ UTR). In some embodiments, the non-coding sequence comprises a five prime untranslated region (5′ UTR). In some embodiments, the editing of the base in the 5′UTR of the region of the target RNA results in at least partially regulating gene translation of the LRRK2 polypeptide. In some embodiments, the editing of the base in the 5′UTR of the region of the target RNA results in facilitating regulation mRNA translation of: the LRRK2 polypeptide. In some embodiments, the target RNA encodes the LRRK2 polypeptide. In some embodiments, the target RNA that encodes the LRRK2 polypeptide comprises at least a portion of: a poly(A) tail, a microRNA response element (MRE), AU-rich element (ARE), hnRNP binding sites or any combination thereof. In some embodiments, the engineered polynucleotide is configured to modulate expression of the LRRK2 polypeptide. In some embodiments, the target RNA encodes a repeat domain of the LRRK2 polypeptide, a Ras-of-complex (Roc) GTPase domain of the LRRK2 polypeptide, a kinase domain of the LRRK2 polypeptide, a WD40 domain of the LRRK2 polypeptide, or a C-terminal of Roc (COR) domain of the LRRK2 polypeptide. In some embodiments, the target RNA encodes the kinase domain of the LRRK2 polypeptide. In some embodiments, the region of the target RNA comprises a mutation as compared to an otherwise comparable region encoding a wildtype polypeptide. In some embodiments, the region of the target RNA comprises a mutation as compared to an otherwise comparable region encoding a wildtype LRRK2 polypeptide. In some embodiments, the mutation comprises a polymorphism. In some embodiments, the mutation is a G to A mutation. In some embodiments, the target RNA comprises at least 80%, 90%, 95%, 97%, 98%, 99%, or 100% sequence identity to any one of SEQ ID NO: 5-SEQ ID NO: 14. In some embodiments, the target RNA encodes a LRRK2 polypeptide comprising at least 80%, 90%, 95%, 97%, 98%, 99%, or 100% sequence identity to any one of SEQ ID NO: 15-SEQ ID NO: 24. In some embodiments, the target RNA encodes a LRRK2 polypeptide comprising a mutation corresponding a G2019S of SEQ ID NO: 15. In some embodiments, the editing of the base is editing of an A corresponding to the 6055th nucleotide in SEQ ID NO: 5. In some embodiments, the target RNA encodes a LRRK2 polypeptide comprising a mutation corresponding to a mutation of Table 3, or any combination of mutations of Table 3. In some embodiments, the engineered polynucleotide comprises at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182. In some embodiments, when the engineered polynucleotide associates with the region of the target RNA, the association comprises hybridized polynucleotide strands. In some embodiments, the hybridized polynucleotide strands at least in part form a double stranded RNA duplex. In some embodiments, the engineered polynucleotide further comprises a chemical modification. In some embodiments, the engineered polynucleotide comprises RNA, DNA, or both. In some embodiments, the engineered polynucleotide comprises the RNA. In some embodiments, the region of the target RNA comprises a translation initiation site. In some embodiments, the administering comprises administering a therapeutically effective amount of the vector. In some embodiments, the administering at least partially treats or prevents at least one symptom of the disease or the condition in the subject in need thereof. In some embodiments, the vector further comprises or encodes a second engineered polynucleotide. In some embodiments, a method further comprises administering a second vector that comprises or encodes a second engineered polynucleotide. In some embodiments, the second engineered polynucleotide comprises a second targeting sequence that at least partially hybridizes to a region of a second target RNA. In some embodiments, the second targeting sequence of the second engineered polynucleotide is at least partially complementary to the region of the second target RNA. In some embodiments, the second target RNA encodes for a polypeptide that comprises: alpha-synuclein (SNCA), glucosylceramidase beta (GBA), PTEN-induced kinase 1 (PINK1), Tau, biologically active fragment of any of these, or any combination thereof. In some embodiments, the second target RNA encodes for the SNCA polypeptide or biologically active fragment thereof. In some embodiments, the second engineered polynucleotide is configured to facilitate an editing of a base of a nucleotide of a polynucleotide of a region of the second target RNA by the RNA editing entity. In some embodiments, the editing results in reduced expression of a polypeptide encoded by the second target RNA. In some embodiments, the second engineered polynucleotide comprises at least 80%, 90%, 95%, 97%, 98%, 99%, or 100% sequence identity to any one of SEQ ID NO: 25-SEQ ID NO: 33. In some embodiments, the second engineered polynucleotide encodes a SCNA polypeptide comprising at least 80%, 90%, 95%, 97%, 98%, 99%, or 100% sequence identity to any one of SEQ ID NO: 34-SEQ ID NO: 36. In some embodiments, the second engineered polynucleotide encodes a SNCA polypeptide comprising a mutation corresponding to a mutation of Table 6, or any combination of mutations of Table 6. In some embodiments, the second engineered polynucleotide facilitates editing of an Adenosine (A) of a translational initiation site of the second target RNA that encodes a SNCA polypeptide. In some embodiments, the second engineered polynucleotide comprises at least 80%, 90%, 95%, 97%, 98%, 99%, or 100% sequence identity to any one of SEQ ID NO: 37-SEQ ID NO: 48. In some embodiments, the second engineered polynucleotide facilitates editing of an Adenosine (A) of a translational initiation site of the second target RNA that encodes a Tau polypeptide. In some embodiments, the second engineered polynucleotide comprises at least 80%, 90%, 95%, 97%, 98%, 99%, or 100% sequence identity to SEQ ID NO: 49. In some embodiments, the second engineered polynucleotide facilitates editing of an Adenosine (A) of a translational initiation site of the second target RNA that encodes a PINK1 polypeptide. In some embodiments, the second engineered polynucleotide comprises at least 80%, 90%, 95%, 97%, 98%, 99%, or 100% sequence identity to any one of SEQ ID NO: 50-SEQ ID NO: 54. In some embodiments, the second engineered polynucleotide facilitates editing of an Adenosine (A) of a translational initiation site of the second target RNA that encodes a GBA polypeptide. In some embodiments, the second engineered polynucleotide comprises at least 60%, 70%, 80%, 85%, 90%, 95%, 97%, 99%, or 100% sequence identity to any one of: SEQ ID NO: 183-SEQ ID NO: 192. In some embodiments, the disease or condition is of a central nervous system (CNS), gastrointestinal (GI) tract, or both. In some embodiments, the disease is of both, and wherein the disease is Parkinson's Disease. In some embodiments, the disease is of the GI tract, and wherein the disease is Crohn's disease. In some embodiments, a method further comprises administering a secondary therapy. In some embodiments, the secondary therapy is administered concurrent or sequential to the vector. In some embodiments, the secondary therapy comprises at least one of a probiotic, a carbidopa, a levodopa, a MAO B inhibitor, a catechol O-methyltransferase (COMT) inhibitor, a anticholinergic, a amantadine, a deep brain stimulation, a salt of any of these, or any combination thereof. In some embodiments, the administering of the vector, the secondary therapy, or both are independently performed at least about: 1 time per day, 2 times per day, 3 times per day, 4 times per day, once a week, twice a week, 3 times a week, biweekly, bimonthly, monthly, or yearly. In some embodiments, a method further comprises monitoring the disease or condition of the subject. In some embodiments, the vector is comprised in a pharmaceutical composition in unit dose form. In some embodiments, the subject is diagnosed with the disease or the condition prior to the administering. In some embodiments, the diagnosing is via an in vitro assay. In some embodiments, the editing of the base of the nucleotide of the polynucleotide of the region of the target RNA comprises at least about 3%, 5%, 10%, 15%, or 20% editing as measured by sequencing. In some embodiments, the second target RNA encodes for the SNCA polypeptide, and wherein the editing of the base of the nucleotide of the polynucleotide of the region of the target RNA by an ADAR polypeptide results in a modified polypeptide that comprises a change in a residue, as compared to an unmodified polypeptide encoded by the target RNA, that comprises: (a) an adenine to an inosine at a position corresponding to position 2019 of the LRRK2 polypeptide of SEQ ID NO: 15; (b) an adenine to an inosine at a position corresponding to position 30 or 53 of the SNCA polypeptide of SEQ ID NO: 34; or (c) (a) and (b).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a gel electrophoresis image of the in vitro transcribed (IVT) templates for various anti-LRRK2 guide RNAs, as amplified by Q5 PCR. The primers listed in Table 12 were used for the amplification. Wt 0.100.50 is LRRK2_0.100.50 (no GluR2 domain; guide is 100 nucleotides in length; A to be edited in the target LRRK2 RNA is positioned at nucleotide 50 of the guide), intGluR2 is LRRK2_IntGluR2, flip_intGluR2 is LRRK2_FlipIntGluR2, Nat guided is LRRK2_Natguide, EIE is LRRK2_EIE, Wt 1.100.50 is LRRK2_1.100.50, and Wt 2.100.50 is LRRK2_2.100.50. The lane on the far left-hand side is the molecular marker.

FIG. 2 shows a gel electrophoresis image of various purified IVT-produced anti-LRRK2 guide RNAs. 25 nmol of RNA was loaded in each lane. Wt 0.100.50 is LRRK2_0.100.50, intGluR2 is LRRK2_IntGluR2, flip_intGluR2 is LRRK2_FlipIntGluR2, Nature guided is LRRK2_Natguide, EIE is LRRK2_EIE, Wt 1.100.50 is LRRK2_1.100.50, and Wt 2.100.50 is LRRK2_2.100.50. The lane on the far left-hand side is the molecular marker. The guide RNA sequences are shown in Table 13.

FIG. 3A-FIG. 3H show the secondary structures of RNA-RNA duplex molecules formed from the binding of different engineered polynucleotides to their target strands. FIG. 3A. shows the secondary structure of an RNA-RNA duplex molecule formed from the binding of LRRK2_0.100.50 to its target strand RNA. The A on the target strand being targeted for editing is marked by an arrow. The 5′ and 3′ end of LRRK2_0.100.50 is shown on the left-hand side and right-hand side, respectively. FIG. 3B shows the secondary structure of an RNA-RNA duplex molecule formed from the binding of LRRK2_1.100.50 to its target strand RNA. The A on the target strand being targeted for editing is marked by an arrow. The 5′ and 3′ end of LRRK2_1.100.50 is shown on the left-hand side and right-hand side, respectively. The 3′ end of LRRK2_1.100.50 also contains a GluR2 hairpin. FIG. 3C shows the secondary structure of an RNA-RNA duplex molecule formed from the binding LRRK2_1.100.50 to its target strand RNA. The A on the target strand being targeted for editing is marked by an arrow. The 5′ and 3′ end of LRRK2_2.100.50 is shown on the left-hand side and right-hand side, respectively. Each of the 5′ and 3′ end of LRRK2_1.100.50 also contains a GluR2 hairpin. FIG. 3D shows the secondary structure of an RNA-RNA duplex molecule formed from the binding LRRK2_IntGluR2 to its target strand RNA. The A on the target strand being targeted for editing is marked by an arrow. The 5′ and 3′ end of LRRK2_IntGluR2 is shown on the left-hand side and right-hand side, respectively. The GluR2 hairpin of LRRK2_IntGluR2 is magnified. FIG. 3E shows the secondary structure of an RNA-RNA duplex molecule formed from the binding LRRK2_FlipIntGluR2 to its target strand RNA. The A on the target strand being targeted for editing is marked by an arrow. The 5′ and 3′ end of LRRK2_FlipIntGluR2 is shown on the left-hand side and right-hand side, respectively. LRRK2_FlipIntGluR2 also contains a hairpin “Flipped” GluR2 hairpin. Its sequence orientation is reversed, as compared to that of LRRK2_IntGluR2. FIG. 3F shows the secondary structure of an RNA-RNA duplex molecule formed from the binding LRRK2 NatGuide to its target strand RNA. The A on the target strand being targeted for editing is marked by an arrow. The 5′ and 3′ end of LRRK2 NatGuide is shown on the left-hand side and right-hand side, respectively. The duplex molecule contains a series of bulges. FIG. 3G shows the secondary structure of an RNA-RNA duplex molecule formed from the binding LRRK2_EIE to its target strand RNA. The A on the target strand being targeted for editing is marked by an arrow. The 5′ and 3′ end of LRRK2_EIE is shown on the left-hand side and right-hand side, respectively. The duplex molecule contains a series of bulges. FIG. 3H shows the secondary structure of an RNA-RNA duplex molecule formed from the binding LRRK2 EIEv2 to its target strand RNA. The A on the target strand being targeted for editing is marked by an arrow. The 5′ and 3′ end of LRRK2 EIEv2 is shown on the left-hand side and right-hand side, respectively. The duplex molecule contains a series of bulges.

FIG. 4 shows Sanger sequencing traces of the 6,055^{th} nucleotide in the LRRK2 G2019S heterozygote cells treated with different anti-LRRK2 guide RNAs (e.g., engineered polynucleotides targeting a region of LRRK2 mRNA) and controls. The cells were contracted with the guide RNAs for 3 hours (left panel) or 7 hours (right panel). The cells were EBV transformed B cells heterozygous for the G2019S mutation. The cells were treated with different guide RNAs. The RNA editing efficiency was calculated by the difference of the trace signal of the LRRK2 mRNA with a G (edited) and an A (unedited). The trace signal was measured by Sanger sequencing. By 3 hours (left panel), the RNA editing efficiency of LRRK2_FlipIntGluR2 (labeled as IntFlip) reached ˜14%, as opposed to 0% in Control (Ctrl). By 7 hours (right panel), other guide RNAs, such as LRRK2_0.100.50 (labeled as 0.100.50) and LRRK2_1.100.50 (labeled as 1.100.50), also showed ˜12% and 13.5% editing, respectively.

FIG. 5A-FIG. 5D show U7-driven expression of engineered guide RNAs with a 3′ SmOPT and U7 hairpin that enhance specific guide RNA editing at additional gene targets with minimal unintended exon skipping. FIG. 5A shows the exon structure of human SNCA. Exons are shown as segments; the coding region is denoted as a black line above. Locations of the guide RNA targeting sites are shown as arrows; PCR primers are shown at the top. FIG. 5B shows ADAR editing at each target site (measured by Sanger sequencing). FIG. 5C shows cDNA from edited transcripts for RAB7a (left) and SNCA (right) were PCR amplified using the above primers and analyzed on an agarose gel. PCR amplicons showed the predicted size for correctly spliced exons. FIG. 5D shows Sanger sequencing chromatograms show specific editing at the target adenosine of the indicated SNCA transcripts (box).

FIG. 6A-FIG. 6C show editing of the 3′ UTR of SNCA. FIG. 6A shows an example Sanger sequencing chromatogram of the edited sites of the 3′ UTR, as well as, off-target editing that can occur. FIG. 6B shows the mouse or human U7 promoter with 3′ SmOPT U7 hairpin constructs of the human SNCA 3′UTR target site, with or without ADAR2 overexpression, in a different cell type (K562-VPR-SNCA) under different transfection conditions (nucleofection, Lonza). FIG. 6C shows the percentage of off target editing occurring at the 5′G in the 3′ UTR using the same constructs as FIG. 6B.

FIG. 7A shows a representative vector map of STB026 mU7 GG U7 deoxyribonucleotides_mU6_CMV GFP sv40. FIG. 7B shows a representative vector map of STX0364 pAAV_hU6 scarless-Bsal_mU6-Bbsl_CMV_GFP.noBbsl. FIG. 7C shows a representative vector map of STX441 pAAV_hU6_circular-spacer2A_mU6_CMV_GFP.

FIG. 8 shows the editing kinetics of different guide RNAs on a target RNA LRRK2. The percent editing of the target gene is indicated on the Y-axis and the time is shown on the X-axis. Three examples of guide RNAs are shown: a guide RNA with a perfect duplex, a guide RNA with a single A-C mismatch, and a top-ranked engineered guide RNA. The top ranked guide RNA had higher percent editing in a shorter amount of time compared to the other guide RNA designs.

FIG. 9 shows the editing kinetics of different guide RNAs on a target RNA LRRK2. The percent editing of the target gene is indicated on the Y-axis and the time is shown on the X-axis. Three examples of guide RNAs are shown: a top-ranked engineered guide RNA, a guide RNA with a single A-C mismatch, and a guide RNA with a perfect duplex. The top ranked guide RNA had 30-fold increase in K_{obs} compared to other guide RNA designs.

FIG. 10A shows the target base editing frequency of various positions of a target RNA LRRK2 using the perfect duplex guide RNA design or the A-C mismatch guide design and ADAR2. The Y-axis shows the percent editing frequency of various positions of the target RNA. The X-axis shows various positions of the target RNA. The arrow indicates the target base A. The top panel shows the target base editing frequency of a perfect duplex guide RNA with the target RNA. The bottom panel shows the target base editing frequency of a A-C mismatch guide RNA at the target A in the target RNA. The on-target target base editing is less than about 20% for either guide RNAs. FIG. 10B shows the target base editing frequency of various positions of a target RNA LRRK2 using a top-ranked engineered design and ADAR2. The Y-axis shows the percent editing frequency of various positions of the target RNA. The X-axis shows various positions of the target RNA. The arrow indicates the target base A. The on-target target base editing is more than 80%.

FIG. 11 shows constructs of piggyBac vectors carrying a LRRK2 minigene having a G2019S mutation and mCherry (at top) or a carrying a LRRK2 minigene having a G2019S mutation, mCherry, CMV, and ADAR2 (at bottom).

FIG. 12A shows in vitro on and off-target editing of the LRRK2 G2019S mutation by ADAR1 after administration of two guide RNAs and a control (GFP plasmid). FIG. 12B shows in vitro on and off-target editing of the LRRK2 G2019S mutation by ADAR1 and ADAR2 after administration of two guide RNAs and a control (GFP plasmid).

FIG. 13 shows graphs of on-target and off-target ADAR1 and ADAR1+ADAAR2 editing of LRRK2 and depicts a circular LRRK2 guide (0.100.80) used in the experiment.

FIG. 14 shows an exemplary control guide RNA Guide 02 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 15 shows the kinetics of editing for the exemplary control guide RNA Guide 02 design for targeting LRRK2.

FIG. 16 shows percentage editing as a function of time for the exemplary control guide RNA Guide 02 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 17 shows an exemplary control guide RNA Guide 03 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 18 shows the kinetics of editing for the exemplary control guide RNA Guide 03 design for targeting LRRK2.

FIG. 19 shows percentage editing as a function of time for the exemplary control guide RNA Guide 03 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 20 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 21 shows the kinetics of editing for the exemplary guide RNA Guide 10 design for targeting LRRK2.

FIG. 22 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 23 shows an exemplary guide RNA Guide 11 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 24 shows the kinetics of editing for the exemplary guide RNA Guide 11 design for targeting LRRK2.

FIG. 25 shows percentage editing as a function of time for the exemplary guide RNA Guide 11 design as determined by sequencing at time points 1m, 10m, 30m, and 100m, and the editing at the target A to be edited (“0” on the x-axis) and at RNA editing at off-target positions (represented as black bars at positions that are not “0”).

FIG. 26 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 27 shows the kinetics of editing for the exemplary guide RNA Guide 10 design for targeting LRRK2.

FIG. 28 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 29 shows an exemplary guide RNA Guide 04 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 30 shows the kinetics of editing for the exemplary guide RNA Guide 04 design for targeting LRRK2.

FIG. 31 shows percentage editing as a function of time for the exemplary guide RNA Guide 04 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 32 shows an exemplary guide RNA Guide 04 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 33 shows the kinetics of editing for the exemplary guide RNA Guide 04 design for targeting LRRK2.

FIG. 34 shows percentage editing as a function of time for the exemplary guide RNA Guide 04 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 35 shows an exemplary guide RNA Guide 11 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 36 shows the kinetics of editing for the exemplary guide RNA Guide 11 design for targeting LRRK2.

FIG. 37 shows percentage editing as a function of time for the exemplary guide RNA Guide 11 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 38 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 39 shows the kinetics of editing for the exemplary guide RNA Guide 10 design for targeting LRRK2.

FIG. 40 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 41 shows an exemplary guide RNA Guide 10 design for targeting LRRK2, the percentage editing as a function of time for each guide RNA as determined by sequencing, and editing at the target A to be edited (“0” on the x-axis) and at RNA editing at off-target positions (represented as black bars at positions that are not “0”).

FIG. 42 shows the kinetics of editing for the exemplary guide RNA Guide 10 design for targeting LRRK2.

FIG. 43 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 44 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 45 shows the kinetics of editing for the exemplary guide RNA Guide 10 design for targeting LRRK2.

FIG. 46 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 47 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 48 shows the kinetics of editing for the exemplary guide RNA Guide 10 design for targeting LRRK2.

FIG. 49 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 50 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 51 shows the kinetics of editing for the exemplary guide RNA Guide 10 design for targeting LRRK2.

FIG. 52 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 53 shows an exemplary guide RNA Guide 11 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 54 shows the kinetics of editing for the exemplary guide RNA Guide 11 design for targeting LRRK2.

FIG. 55 shows percentage editing as a function of time for the exemplary guide RNA Guide 11 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 56 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 57 shows the kinetics of editing for the exemplary guide RNA Guide 10 design for targeting LRRK2.

FIG. 58 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 59 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 60 shows the kinetics of editing for the exemplary guide RNA Guide 10 design for targeting LRRK2.

FIG. 61 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 62 shows an exemplary guide RNA Guide 11 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 63 shows the kinetics of editing for the exemplary guide RNA Guide 11 design for targeting LRRK2.

FIG. 64 shows percentage editing as a function of time for the exemplary guide RNA Guide 11 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 65 shows an exemplary guide RNA Guide 11 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 66 shows the kinetics of editing for the exemplary guide RNA Guide 11 design for targeting LRRK2.

FIG. 67 shows percentage editing as a function of time for the exemplary guide RNA Guide 11 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 68 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 69 shows the kinetics of editing for the exemplary guide RNA Guide 10 design for targeting LRRK2.

FIG. 70 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 71 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 72 shows the kinetics of editing for the exemplary guide RNA Guide 10 design for targeting LRRK2.

FIG. 73 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 74 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 75 shows the kinetics of editing for the exemplary guide RNA Guide 10 design for targeting LRRK2.

FIG. 76 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 77 shows an exemplary guide RNA Guide 04 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 78 shows the kinetics of editing for the exemplary guide RNA Guide 04 design for targeting LRRK2.

FIG. 79 shows percentage editing as a function of time for the exemplary guide RNA Guide 04 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 80 shows an exemplary guide RNA Guide 11 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 81 shows the kinetics of editing for the exemplary guide RNA Guide 11 design for targeting LRRK2.

FIG. 82 shows percentage editing as a function of time for the exemplary guide RNA Guide 11 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 83 shows an exemplary guide RNA Guide 11 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 84 shows the kinetics of editing for the exemplary guide RNA Guide 11 design for targeting LRRK2.

FIG. 85 shows percentage editing as a function of time for the exemplary guide RNA Guide 11 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 86 shows an exemplary guide RNA Guide 11 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 87 shows the kinetics of editing for the exemplary guide RNA Guide 11 design for targeting LRRK2.

FIG. 88 shows percentage editing as a function of time for the exemplary guide RNA Guide 11 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 89 shows an exemplary guide RNA Guide 03 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 90 shows the kinetics of editing for the exemplary guide RNA Guide 03 design for targeting LRRK2.

FIG. 91 shows percentage editing as a function of time for the exemplary guide RNA Guide 03 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 92 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 93 shows the kinetics of editing for the exemplary guide RNA Guide 10 design for targeting LRRK2.

FIG. 94 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 95 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 96 shows the kinetics of editing for the exemplary guide RNA Guide 10 design for targeting LRRK2.

FIG. 97 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 98 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 99 shows the kinetics of editing for the exemplary guide RNA Guide 10 design for targeting LRRK2.

FIG. 100 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 101 shows an exemplary guide RNA Guide 11 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 102 shows the kinetics of editing for the exemplary guide RNA Guide 11 design for targeting LRRK2.

FIG. 103 shows percentage editing as a function of time for the exemplary guide RNA Guide 11 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 104 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 105 shows the kinetics of editing for the exemplary guide RNA Guide 10 design for targeting LRRK2.

FIG. 106 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 107 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 108 shows the kinetics of editing for the exemplary guide RNA Guide 10 design for targeting LRRK2.

FIG. 109 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 110 shows an exemplary guide RNA Guide 10 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 111 shows the kinetics of editing for the exemplary guide RNA Guide 10 design for targeting LRRK2.

FIG. 112 shows percentage editing as a function of time for the exemplary guide RNA Guide 10 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 113 shows an exemplary guide RNA Guide 11 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 114 shows the kinetics of editing for the exemplary guide RNA Guide 11 design for targeting LRRK2.

FIG. 115 shows percentage editing as a function of time for the exemplary guide RNA Guide 11 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 116 shows an exemplary guide RNA Guide 11 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 117 shows the kinetics of editing for the exemplary guide RNA Guide 11 design for targeting LRRK2.

FIG. 118 shows percentage editing as a function of time for the exemplary guide RNA Guide 11 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 119 shows an exemplary guide RNA Guide 11 design for targeting LRRK2, the percentage editing as a function of time for each guide RNA as determined by sequencing, and the editing at the target A to be edited (“0” on the x-axis) and at RNA editing at off-target positions (represented as black bars at positions that are not “0”).

FIG. 120 shows the kinetics of editing for the exemplary guide RNA Guide 11 design for targeting LRRK2.

FIG. 121 shows percentage editing as a function of time for the exemplary guide RNA Guide 11 design as determined by sequencing at time points 1 m, 10m, 30m, and 100m, and the editing at the target A to be edited (“0” on the x-axis) and at RNA editing at off-target positions (represented as black bars at positions that are not “0”).

FIG. 122 shows heat maps and structures for exemplary engineered polynucleotide sequences targeting a LRRK2 mRNA. The heat map provides visualization of the editing profile at the 10 minute time point. 5 engineered polynucleotides for on-target editing (with no-2 filter) are in the left graph and 5 engineered polynucleotides for on-target editing with minimal-2 editing are depicted on the right graph. The corresponding predicted secondary structures are below the heat maps.

FIG. 123 shows exemplary engineered polynucleotides comprising a dumbbell design and that target LRRK2 mRNA.

FIG. 124 shows graphs of on-target and off-target ADAR1 (left side) and ADAR1+ADAAR2 (right side) editing of LRRK2 for engineered polynucleotides of FIG. 123.

FIG. 125 shows graphs of on-target and off-target ADAR1 (left side) and ADAR1+ADAAR2 (right side) editing of LRRK2 for engineered polynucleotides of FIG. 123.

FIG. 126 shows graphs of on-target and off-target ADAR1 (left side) and ADAR1+ADAAR2 (right side) editing of LRRK2 for engineered polynucleotides of FIG. 123.

FIG. 127 shows graphs of on-target and off-target ADAR1 (left side) and ADAR1+ADAAR2 (right side) editing of LRRK2 for the engineered polynucleotides of FIG. 123.

### DETAILED DESCRIPTION

The practice of some embodiments disclosed herein employ, unless otherwise indicated, conventional techniques of immunology, biochemistry, chemistry, molecular biology, microbiology, cell biology, genomics and recombinant DNA, which are within the skill of the art.

Unless defined otherwise, all technical and scientific terms used herein have the same meaning as is commonly understood by one of skill in the art.

The term “a” and “an” refers to one or to more than one (i.e., to at least one) of the grammatical object of the article. By way of example, “an element” means one element or more than one element.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values can be described in the application and claims, unless otherwise stated the term “about” meaning within an acceptable error range for the particular value should be assumed. Also, where ranges, subranges, or both, of values can be provided, the ranges or subranges can include the endpoints of the ranges or subranges. The terms “substantially”, “substantially no”, “substantially not”, “substantially free”, and “approximately” can be used when describing a magnitude, a position or both to indicate that the value described can be within a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The term “subject,” “host,” “individual,” and “patient” are as used interchangeably herein to refer to animals, typically mammalian animals. Any suitable mammal can be treated by a method, cell or composition described herein. A mammal can be administered a vector, an engineered guide RNA, a precursor guide RNA, a nucleic acid, a polynucleotide, an engineered polynucleotide, or a pharmaceutical composition, as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a disease such as a neurodegenerative disease. In some embodiments, a subject has or can be suspected of having a cancer or neoplastic disorder. In other embodiments, a subject has or can be suspected of having a disease or disorder associated with aberrant protein expression. In some cases, a human can be more than about: 1 day to about 10 months old, from about 9 months to about 24 months old, from about 1 year to about 8 years old, from about 5 years to about 25 years old, from about 20 years to about 50 years old, from about 1 year old to about 130 years old or from about 30 years to about 100 years old. Humans can be more than about: 1, 2, 5, 10, 20, 30, 40, 50, 60, 70, 80, 90, 100, 110, or 120 years of age. Humans can be less than about: 1, 2, 5, 10, 20, 30, 40, 50, 60, 70, 80, 90, 100, 110, 120 or 130 years of age.

The term “sample” as used herein, generally refers to any sample of a subject (such as a blood sample or a tissue sample). A sample or portion thereof may comprise cell, such as a stem cell. A portion of a sample may be enriched for the stem cell. The stem cell may be isolated from the sample. A sample may comprise a tissue, a cell, serum, plasma, exosomes, a bodily fluid, or any combination thereof. A bodily fluid may comprise urine, blood, serum, plasma, saliva, mucus, spinal fluid, tears, semen, bile, amniotic fluid, cerebrospinal fluid, or any combination thereof. A sample or portion thereof may comprise an extracellular fluid obtained from a subject. A sample or portion thereof may comprise cell-free nucleic acid, DNA or RNA. A sample or portion thereof may be analyzed for a presence or absence or one or more mutations. Genomic data may be obtained from the sample or portion thereof. A sample may be a sample suspected or confirmed of having a disease or condition. A sample may be a sample removed from a subject via a non-invasive technique, a minimally invasive technique, or an invasive technique. A sample or portion thereof may be obtained by a tissue brushing, a swabbing, a tissue biopsy, an excised tissue, a fine needle aspirate, a tissue washing, a cytology specimen, a surgical excision, or any combination thereof. A sample or portion thereof may comprise tissues or cells from a tissue type. For example, a sample may comprise a nasal tissue, a trachea tissue, a lung tissue, a pharynx tissue, a larynx tissue, a bronchus tissue, a pleura tissue, an alveoli tissue, breast tissue, bladder tissue, kidney tissue, liver tissue, colon tissue, thyroid tissue, cervical tissue, prostate tissue, heart tissue, muscle tissue, pancreas tissue, anal tissue, bile duct tissue, a bone tissue, brain tissue, spinal tissue, kidney tissue, uterine tissue, ovarian tissue, endometrial tissue, vaginal tissue, vulvar tissue, uterine tissue, stomach tissue, ocular tissue, sinus tissue, penile tissue, salivary gland tissue, gut tissue, gallbladder tissue, gastrointestinal tissue, bladder tissue, brain tissue, spinal tissue, a blood sample, or any combination thereof.

“Eukaryotic cells” comprise all life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

The term “protein”, “peptide”, and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. As used herein, the term “fusion protein” refers to a protein comprised of domains from more than one naturally occurring or recombinantly produced protein, where generally each domain serves a different function. In this regard, the term “linker” refers to a protein fragment that is used to link these domains together—optionally to preserve the conformation of the fused protein domains and/or prevent unfavorable interactions between the fused protein domains which may compromise their respective functions.

“Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed.

In some cases, the identity between a reference sequence (query sequence, i.e., a sequence of the disclosure) and a subject sequence, also referred to as a global sequence alignment, can be determined using the FASTDB computer program based on the algorithm of Brutlag et al. (Comp. App. Biosci. 6:237-245 (1990)). In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA of a sequence, isolated RNA of a sequence, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA). A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double and single stranded molecules. Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction. Unless otherwise specified or required, any embodiment of this disclosure that is a polynucleotide encompasses both the double stranded form and each of two complementary single stranded forms known or predicted to make up the double stranded form.

Polynucleotides useful in the methods of the disclosure can comprise natural nucleic acid sequences and variants thereof, artificial nucleic acid sequences, or a combination of such sequences. In some embodiments, polynucleotides of the disclosure refer to a DNA sequence. In some embodiments, the DNA sequence is interchangeable with a similar RNA sequence. In some embodiments, polynucleotides of the disclosure refer to an RNA sequence. In some embodiments, the RNA sequence is interchangeable with a similar DNA sequence. In some embodiments, Us and Ts of a polynucleotide may be interchanged in a sequence provided herein.

A polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), a nucleoside formed when hypoxanthine is attached to ribofuranose via a β-N9-glycosidic bond, resulting in the chemical structure:

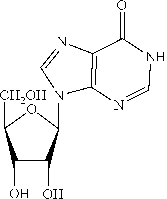

Inosine is read by the translation machinery as guanine (G).

The term “polynucleotide sequence” is the alphabetical representation of a polynucleotide molecule. This alphabetical representation can be input into databases in a computer having a central processing unit and used for bioinformatics applications such as functional genomics and homology searching.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell.

The terms “equivalent” or “biological equivalent” are used interchangeably when referring to a particular molecule, biological, or cellular material and intend those having minimal homology while still maintaining desired structure or functionality.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, the term “functional” may be used to modify any molecule, biological, or cellular material to intend that it accomplishes a particular, specified effect.

The term “mutation” as used herein, refers to an alteration to a nucleic acid sequence encoding a protein relative to the consensus sequence of said protein. “Missense” mutations result in the substitution of one codon for another; “nonsense” mutations change a codon from one encoding a particular amino acid to a stop codon. Nonsense mutations often result in truncated translation of proteins. “Silent” mutations are those which have no effect on the resulting protein. As used herein the term “point mutation” refers to a mutation affecting only one nucleotide in a gene sequence. “Splice site mutations” are those mutations present pre-mRNA (prior to processing to remove introns) resulting in mistranslation and often truncation of proteins from incorrect delineation of the splice site. A mutation can comprise a single nucleotide variation (SNV). A mutation can comprise a sequence variant, a sequence variation, a sequence alteration, or an allelic variant. The reference DNA sequence can be obtained from a reference database. A mutation can affect function. A mutation may not affect function. A mutation can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. A mutation can be a point mutation. A mutation can be a fusion gene. A fusion pair or a fusion gene can result from a mutation, such as a translocation, an interstitial deletion, a chromosomal inversion, or any combination thereof. A mutation can constitute variability in the number of repeated sequences, such as triplications, quadruplications, or others. For example, a mutation can be an increase or a decrease in a copy number associated with a given sequence (e.g., copy number variation, or CNV). A mutation can include two or more sequence changes in different alleles or two or more sequence changes in one allele. A mutation can include two different nucleotides at one position in one allele, such as a mosaic. A mutation can include two different nucleotides at one position in one allele, such as a chimeric. A mutation can be present in a malignant tissue. A presence or an absence of a mutation can indicate an increased risk to develop a disease or condition. A presence or an absence of a mutation can indicate a presence of a disease or condition. A mutation can be present in a benign tissue. Absence of a mutation may indicate that a tissue or sample is benign. As an alternative, absence of a mutation may not indicate that a tissue or sample is benign. Methods as described herein can comprise identifying a presence of a mutation in a sample.

“Messenger RNA” or “mRNA” is a nucleic acid molecule that is transcribed from DNA and then processed to remove non-coding sections known as introns. The resulting mRNA is exported from the nucleus (or another locus where the DNA is present) and translated into a protein. The term “pre-mRNA” refers to the strand prior to processing to remove non-coding sections.

“Non-coding” sections or sequences refer to portions of an RNA polynucleotide that is not translated into a gene. Such non-coding sequences include 5′ and 3′ untranslated sequences such as a Shine-Dalgarno sequence, a Kozak consensus sequence, a 3′ poly-A tail, and the like.

“Canonical amino acids” refer to those 20 amino acids found naturally in the human body shown in the table below with each of their three letter abbreviations, one letter abbreviations, structures, and corresponding codons:

non-polar, aliphatic residues

| | | | | |
|---|---|---|---|---|
| Glycine | Gly | G | 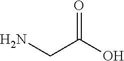 | GGU; GGC; GGA; GGG |
| Alanine | Ala | A | 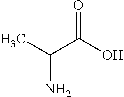 | GCU; GCC; GCA; GCG |
| Valine | Val | V | 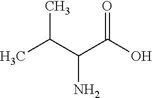 | GUU; GUC; GUA; GUG |
| Leucine | Leu | L | 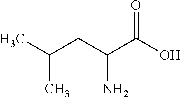 | UUA; UUG; CUU; CUC; CUA; CUG |
| Isoleucine | Ile | I | 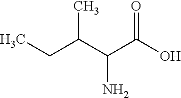 | AUU; AUC; AUA |
| Proline | Pro | P | 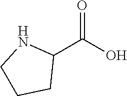 | CCU; CCC; CCA; CCG |

aromatic residues

| | | | | |
|---|---|---|---|---|
| Phenylalanine | Phe | F | 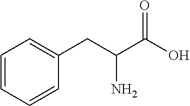 [SMILES: NC(Cc1ccccc1)C(=O)O] | UUU; UUC |
| Tyrosine | Tyr | Y | 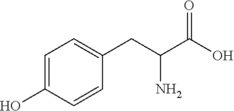 [SMILES: NC(Cc1ccc(O)cc1)C(=O)O] | UAU; UAC |
| Tryptophan | Trp | W | 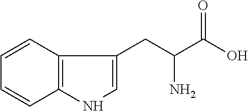 [SMILES: NC(Cc1c[nH]c2ccccc12)C(=O)O] | UGG |

polar, non-charged residues

| | | | | |
|---|---|---|---|---|
| Serine | Ser | S | 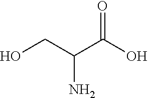 | UCU; UCC; UCA UCG AGU AGC |
| Threonine | Thr | T | 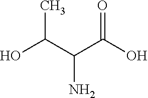 | ACU; ACC; ACA; ACG |
| Cysteine | Cys | C | 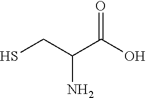 | UGU; UGC |
| Methionine | Met | M | 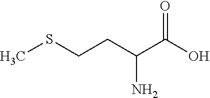 | AUG |
| Asparagine | Asn | N | 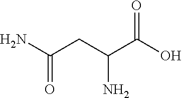 | AAU; AAC |
| Glutamine | Gln | Q | 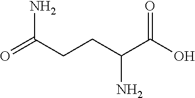 | CAA; CAG |

positively charged residues

| | | | | |
|---|---|---|---|---|
| Lysine | Lys | K | 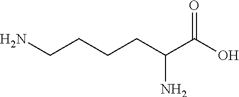 | AAA; AAG |
| Arginine | Arg | R | 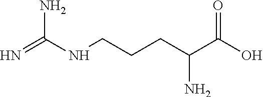 [SMILES: N=C(N)NCCCC(N)C(=O)O] | CGU; CGC; CGA; CGG; AGA; AGG |
| Histidine | His | H | 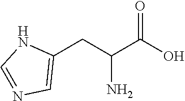 [SMILES: NC(Cc1cnc[nH]1)C(=O)O] | CAU;CAC |

negatively charged residues

| | | | | |
|---|---|---|---|---|
| Aspartate | Asp | D | 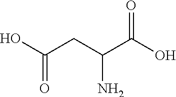 | GAU; GAC |
| Glutamate | Glu | E | 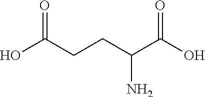 | GAA; GAG |

The term “non-canonical amino acids” refers to those synthetic or otherwise modified amino acids that fall outside this group, typically generated by chemical synthesis or modification of canonical amino acids (e.g. amino acid analogs). The present disclosure employs proteinogenic non-canonical amino acids in some of the methods and vectors disclosed herein. A non-limiting example of a non-canonical amino acid is pyrrolysine (Pyl or 0), the chemical structure of which is provided below:

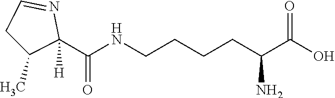 [SMILES: C[C@@H]1CC=N[C@H]1C(=O)NCCCC[C@H](N)C(=O)O]

Inosine (I) is another exemplary non-canonical amino acid, which is commonly found in tRNA and is essential for proper translation according to “wobble base pairing.” The structure of inosine is provided above.

The term “ADAR” as used herein refers to an Adenosine Deaminase Acting on RNA that can convert adenosines (A) to inosines (I) in an RNA sequence. ADAR1 and ADAR2 are two exemplary species of ADAR that are involved in RNA editing in vivo. Non-limiting exemplary sequences for ADAR1 may be found under the following reference numbers: HGNC: 225; Entrez Gene: 103; Ensembl: ENSG 00000160710; OMIM: 146920; UniProtKB: P55265; and GeneCards: GC01M154554, as well as biological equivalents thereof. Non-limiting exemplary sequences for ADAR2 may be found under the following reference numbers: HGNC: 226; Entrez Gene: 104; Ensembl: ENSG00000197381; OMIM: 601218; UniProtKB: P78563; and GeneCards: GC21P045073, as well as biological equivalents thereof. Biologically active fragments of ADAR are also provided herein and can be included when referring to an ADAR.

The term “deficiency” as used herein refers to lower than normal (physiologically acceptable) levels of a particular agent. In context of a protein, a deficiency refers to lower than normal levels of the full-length protein.

The term “complementary” or “complementarity” refers to the ability of a nucleic acid to form hydrogen bond(s) with another nucleic acid sequence by either traditional Watson-Crick or other non-traditional types. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary”, “partially complementary”, “at least partially complementary”, or as used herein refers to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or refers to two nucleic acids that hybridize under stringent conditions (e.g., stringent hybridization conditions). Nucleic acids can include nonspecific sequences. As used herein, the term “nonspecific sequence” or “not specific” refers to a nucleic acid sequence that contains a series of residues that can be not designed to be complementary to or can be only partially complementary to any other nucleic acid sequence.

As used herein, the term “domain” refers to a particular region of a protein or polypeptide and can be associated with a particular function. For example, “a domain which associates with an RNA hairpin motif” refers to the domain of a protein that binds one or more RNA hairpin. This binding may optionally be specific to a particular hairpin.

It is to be inferred without explicit recitation and unless otherwise intended, that when the present disclosure relates to a polypeptide, protein, polynucleotide or antibody, an equivalent or a biological equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent can have at least about 70% homology or identity, at least 80% homology or identity, at least about 85%, at least about 90%, at least about 95%, or at least about 98% percent homology or identity and exhibits substantially equivalent biological activity to the reference protein, polypeptide, or nucleic acid. Alternatively, when referring to polynucleotides, an equivalent thereof is a polynucleotide that hybridizes under stringent conditions to the reference polynucleotide or its complement.

The section headings used herein are for organizational purposes and are not to be construed as limiting the subject matter described.

### Overview

RNA editing has emerged as an attractive alternative to DNA editing. Unlike DNA editing, RNA editing may be less likely to cause a potentially dangerous immune reaction such as those reported utilizing CRISPR-based approaches. Indeed, unlike the DNA-editing enzyme Cas9, which comes from bacteria, RNA editing entities and biologically active fragments thereof such as Adenosine Deaminase Acting on RNA (ADAR) are human proteins that do not trigger the adaptive immune system. Additionally, RNA editing may be a safer approach to gene therapies because editing RNA does not contain a risk for permanent genomic changes as seen with DNA editing. Also, while off-site RNA editing may occur, the off-site edited mRNA is diluted out and/or degraded, unlike with off-site DNA editing that is permanent, e.g., the transient nature of pre-mRNA and mRNA compared to the permeance of DNA, off-site editing is likely far less consequential in the context of RNA vs DNA.

Provided herein are compositions and methods for use in targeting an RNA, particularly for the prevention, amelioration, and/or treatment of disease. Although many diseases can be targeted utilizing the compositions and methods provided herein, in some embodiments, those associated with mutations in Leucine-rich repeat kinase 2 (LRRK2) are targeted. LRRK2 mutations are associated with diseases arising in the central nervous system (CNS) and gastrointestinal (GI) tract. In an aspect, the compositions and methods of the disclosure provide suitable means for which to treat CNS and/or GI disease with improved targeting and reduced immunogenicity as compared to available technologies utilizing DNA editing. In some embodiments, diseases associated with Alpha Synuclein (SNCA) are targeted. In some embodiments, diseases associated with Glucosylceramidase Beta (GBA) are targeted. In some embodiments, diseases associated with PTEN-induced Kinase 1 (PINK1) are targeted. In some embodiments, diseases associated with Tau encoded by MAPT are targeted.

### Targeting of Ribonucleic Acid

Targeting an RNA can be a process by which RNA can be enzymatically modified post synthesis on specific nucleosides. Targeting of RNA can comprise any one of an insertion, deletion, or substitution of a nucleotide(s). Examples of RNA targeting include pseudouridylation (the isomerization of uridine residues) and deamination (removal of an amine group from cytidine to give rise to uridine (C-to-U editing); or removal of an amine group from adenosine to inosine (A-to-I editing)).

Targeting of RNA can modulate expression of a polypeptide. For example, through modulation of polypeptide-encoding dsRNA substrates that enter the RNA interference (RNAi) pathway. This modulation may be by small interfering RNAs (siRNA) that act at the chromatin level to modulate expression of the polypeptide. This modulation may be by micro RNAs (miRNA) that act at the RNA level to modulate expression of the polypeptide.

Targeting of RNA can also be a way to regulate translation of an RNA transcribe form a gene. RNA editing can be a mechanism in which to regulate transcript recoding, e.g., by regulating the introduction of silent mutations and/or non-synonymous mutations into a triplet codon of a transcript.

### RNA Editing Entities and Biologically Active Fragments Thereof

Provided herein are compositions that comprise an RNA editing entity or a biologically active fragment thereof and methods of using the same. An RNA editing entity or biologically active fragment thereof can be any enzyme or biologically fragment thereof that comprises a catalytic domain for catalyzing the chemical conversion of an adenosine to an inosine in RNA.

In an aspect, an RNA editing entity can comprise an adenosine Deaminase Acting on RNA (ADAR), Adenosine Deaminase Acting on tRNA (ADAT), or a biologically active fragment thereof of either of these. ADARs and ADATs can be enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR and ADAT enzymes share a similar single carboxy-terminal catalytic deaminase domain.

ADAR can comprise a variable number of amino-terminal dsRNA binding domains (dsRBDs) and a single carboxy-terminal catalytic deaminase domain. Human ADARs possess two or three dsRBDs. Evidence suggests that ADARs can form homodimer as well as heterodimer with other ADARs when bound to double-stranded RNA, however it is currently inconclusive if dimerization is required for editing to occur. Three human ADAR genes have been identified (ADARs 1-3) with ADAR1 (ADAR) and ADAR2 (ADARB1) proteins having well-characterized adenosine deamination activity. ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain. In an aspect, an RNA editing entity comprises an ADAR. In some embodiments, an ADAR can comprise any one of: ADAR1, ADAR1p110, ADAR1p150, ADAR2, ADAR3, APOBEC protein, or any combination thereof. In some embodiments, the ADAR RNA editing entity is ADAR1. Additionally, or alternatively, the ADAR RNA editing entity is ADAR2. Additionally, or alternatively, the ADAR RNA editing entity is ADAR3. In an aspect, an RNA editing entity can be a non-ADAR In some cases, an RNA editing entity can comprise at least about 80% sequence homology to APOBEC1, APOBEC2, ADAR1, ADAR1p110, ADAR1p150, ADAR2, ADAR3, or any combination thereof.

ADAT catalyzes the deamination on tRNAs. ADAT is also named tadA in E. coli. Three human ADAT genes have been identified (ADATs 1-3).

Specific RNA editing can lead to transcript recoding. Because inosine shares the base pairing properties of guanosine, the translational machinery interprets edited adenosines as guanosine, altering the triplet codon, which can result in amino acid substitutions in protein products. More than half the triplet codons in the genetic code can be reassigned through RNA editing. Due to the degeneracy of the genetic code, RNA editing can cause both silent and non-synonymous amino acid substitutions.

In some cases, targeting an RNA can affect splicing. Adenosines targeted for editing may be disproportionately localized near splice junctions in pre-mRNA. Therefore, during formation of a dsRNA ADAR substrate, intronic cis-acting sequences can form RNA duplexes encompassing splicing sites and potentially obscuring them from the splicing machinery. Furthermore, through modification of select adenosines, ADARs can create or eliminate splicing sites, broadly affecting later splicing of the transcript. Similar to the translational machinery, the spliceosome interprets inosine as guanosine, and therefore, a canonical GU 5′ splice site and AG 3′ acceptor site can be created via the deamination of AU (IU=GU) and AA (AI=AG), respectively. Correspondingly, RNA editing can destroy a canonical AG 3′ splice site (IG=GG).

In some cases, targeting an RNA can affect microRNA (miRNA) production and function. For example, RNA editing of a pre-miRNA precursor can affect the abundance of an miRNA, RNA editing in the seed of the miRNA can redirect it to another target for translational repression, or RNA editing of a miRNA binding site in an RNA can interfere with miRNA complementarity, and thus interfere with suppression via RNAi.

Alternate RNA editing entities are also contemplated, such as those from a clustered regularly interspaced short palindromic repeats (CRISPR) system, such as Cas13 (e.g., Cas13a, Cas13b, Cas13c, Cas13d).

In some cases, an RNA editing entity can be a virus-encoded RNA-dependent RNA polymerase. In some cases, an RNA editing entity can be a virus-encoded RNA-dependent RNA polymerase from measles, mumps, or parainfluenza. In some instances, an RNA editing entity can be an enzyme from Trypanosoma brucei capable of adding or deleting a nucleotide or nucleotides in a target RNA. In some instances, an RNA editing entity can be an enzyme from Trypanosoma brucei capable of adding or deleting an Uracil or more than one Uracil in a target RNA. In some instances, an RNA editing entity can comprise a recombinant enzyme. In some cases, an RNA editing entity can comprise a fusion polypeptide.

In an aspect, an RNA editing entity can be recruited by an engineered polynucleotide as disclosed herein to at target RNA. In some embodiments, an engineered polynucleotide can recruit an RNA editing entity to a target RNA that, when the RNA editing entity is associated with the engineered polynucleotide and the target RNA, facilitates: an editing of a base of a nucleotide of a polynucleotide of the region of the target RNA, a modulation of the expression of a polypeptide encoded by the target RNA, such as LRRK2, SNCA, PINK1, Tau; or a combination thereof. An engineered polynucleotide can comprise an RNA editing entity recruiting domain capable of recruiting an RNA editing entity. In some embodiments, an engineered polynucleotide can lack an RNA editing entity recruiting domain and still be capable of binding an RNA editing entity, or be bound by it.

### Engineered Polynucleotides

Provided herein are polynucleotides and compositions that comprise the same. In an aspect, a polynucleotide can be an engineered polynucleotide. In an embodiment, an engineered polynucleotide can be an engineered polyribonucleotide. In some embodiments, an engineered polynucleotide of the disclosure may be utilized for RNA editing, for example to prevent or treat a disease or condition. In some cases, an engineered polynucleotide can be used in association with a subject RNA editing entity to edit a target RNA or modulate expression of a polypeptide encoded by the target RNA. In an embodiment, compositions disclosed herein can include engineered polynucleotides capable of facilitating editing by subject RNA editing entities such as ADAR or ADAT polypeptides or biologically active fragments thereof.

Engineered polynucleotides can be engineered in any way suitable for RNA targeting. In an aspect, an engineered polynucleotide generally comprises at least a targeting sequence that allows it to hybridize to a region of a target RNA. In some embodiments, the targeting sequence partially hybridizes to a region of a target RNA. In some cases, a targeting sequence may also be referred to as a targeting domain or a targeting region.

In an aspect, a targeting sequence of an engineered polynucleotide allows the engineered polynucleotide to target an RNA sequence through base pairing, such as Watson Crick base pairing. In an embodiment, the targeting sequence can be located at either the N-terminus or C-terminus of the engineered polynucleotide. In some cases, the targeting sequence is located at both termini. The targeting sequence can be of any length. In some cases, the targeting sequence is at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In an embodiment, an engineered polynucleotide comprises a targeting sequence that is about 75-100, 80-110, 90-120, or 95-115 nucleotides in length. In an embodiment, an engineered polynucleotide comprises a targeting sequence that is about 100 nucleotides in length. In an embodiment, an engineered polynucleotide comprises a targeting sequence that is from 50-200, 50-300, or 80-120 nucleotides in length.

In some cases, a subject targeting sequence comprises at least partial sequence complementarity to a region of a target RNA. In some embodiments, the target RNA comprises an mRNA sequence. In some embodiments, the mRNA sequence comprises coding and non-coding sequence. In some embodiments, the non-coding sequence comprises a five prime untranslated region (5′UTR), a three prime untranslated region (5′UTR), an intron, or any combination thereof. In some embodiments, the mRNA sequence encodes a subject polypeptide, for example LRRK2, SNCA, GBA, PINK1, or Tau. In some embodiments, the region of the target RNA comprises from 5 to 400 nucleotides from an mRNA sequence, wherein the mRNA sequence encodes a subject polypeptide, for example LRRK2, SNCA, GBA, PINK1, or Tau. In some embodiments, the region of the target RNA comprises from 5 to 400 nucleotides from a non-coding and coding sequence of an mRNA sequence, wherein the coding sequence encodes a subject polypeptide, for example LRRK2, SNCA, GBA, PINK1, or Tau. In some embodiments, the region of the target RNA comprises from 5 to 400 nucleotides from a three prime untranslated region (3′UTR) and the sequence that encodes a subject polypeptide, for example LRRK2, SNCA, GBA, PINK1, or Tau. In some embodiments, the region of the target RNA comprises from 5 to 300 nucleotides from a five prime untranslated region (5′UTR) and the sequence that encodes a subject polypeptide, for example LRRK2, SNCA, GBA, PINK1, or Tau. In some embodiments, the region of the target RNA comprises from 5 to 400 nucleotides from a three prime untranslated region (3′UTR) and the sequence that encodes a subject polypeptide, for example LRRK2, SNCA, GBA, PINK1, or Tau. In some cases, a subject targeting sequence comprises at least partial sequence complementarity to a region of a target RNA that at least partially encodes a subject polypeptide for example LRRK2, SNCA, GBA, PINK1, or Tau.

In some cases, a targeting sequence comprises 95%, 96%, 97%, 98%, 99%, or 100% sequence complementarity to a region of a target RNA. In some cases, a targeting sequence comprises less than 100% complementarity to a region of a target RNA sequence. For example, a targeting sequence and a region of a target RNA that can be bound by the targeting sequence may have a single base mismatch. In other cases, the targeting sequence of a subject engineered polynucleotide comprises at least about 1, 2, 3, 4, 5, 6, 7, 8, 9, 10, 11, 12, 13, 14, 15, 16, 20, 30, 40 or up to about 50 base mismatches. In some aspects, nucleotide mismatches can be associated with structural features provided herein. In some aspects, a targeting sequence comprises at least about 1, 2, 3, 4, 5, 6, 7, 8, 9, 10, 11, 12, 13, 14, or up to about 15 nucleotides that differ in complementarity from a wildtype RNA of a subject region of a target RNA. In some aspects, a targeting sequence comprises at least about 1, 2, 3, 4, 5, 6, 7, 8, 9, 10, 11, 12, 13, 14, or up to about 15 nucleotides that differ in complementarity from a subject region of a target RNA. In some cases, a targeting sequence comprises at least 50 nucleotides having complementarity to a region of a target RNA. In some cases, a targeting sequence comprises from 50 to 150 nucleotides having complementarity to a region of a target RNA. In some cases, a targeting sequence comprises from 50 to 200 nucleotides having complementarity to a region of a target RNA. In some cases, a targeting sequence comprises from 50 to 250 nucleotides having complementarity to a region of a target RNA. In some cases, a targeting sequence comprises from 50 to 300 nucleotides having complementarity to a region of a target RNA. In some cases, a targeting sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 nucleotides having complementarity to a region of a target RNA. In some cases, a targeting sequence comprises more than 50 nucleotides total and has at least 50 nucleotides having complementarity to a region of a target RNA. In some cases, a targeting sequence comprises from 50 to 400 nucleotides total and has from 50 to 150 nucleotides having complementarity to a region of a target RNA. In some cases, a targeting sequence comprises from 50 to 400 nucleotides total and has from 50 to 200 nucleotides having complementarity to a region of a target RNA. In some cases, a targeting sequence comprises from 50 to 400 nucleotides total and has from 50 to 250 nucleotides having complementarity to a region of a target RNA. In some cases, a targeting sequence comprises from 50 to 400 nucleotides total and has from 50 to 300 nucleotides having complementarity to a region of a target RNA. In some cases, the at least 50 nucleotides having complementarity to a region of a target RNA are separated by one or more structural features. In some cases, the at least 50 nucleotides having complementarity to a region of a target RNA are separated by one or more mismatches, one or more bulges, or one or more loops, or any combination thereof. In some cases, the from 50 to 150 nucleotides having complementarity to a region of a target RNA are separated by one or more structural features. In some cases, the from 50 to 150 nucleotides having complementarity to a region of a target RNA are separated by one or more mismatches, one or more bulges, or one or more loops, or any combination thereof. In some cases, the from 50 to 200 nucleotides having complementarity to a region of a target RNA are separated by one or more structural features. In some cases, the from 50 to 200 nucleotides having complementarity to a region of a target RNA are separated by one or more mismatches, one or more bulges, or one or more loops, or any combination thereof. In some cases, the from 50 to 250 nucleotides having complementarity to a region of a target RNA are separated by one or more structural features. In some cases, the from 50 to 250 nucleotides having complementarity to a region of a target RNA are separated by one or more mismatches, one or more bulges, or one or more loops, or any combination thereof. In some cases, the from 50 to 300 nucleotides having complementarity to a region of a target RNA are separated by one or more structural features. In some cases, the from 50 to 300 nucleotides having complementarity to a region of a target RNA are separated by one or more mismatches, one or more bulges, or one or more loops, or any combination thereof. For example, a targeting sequence comprises a total of 54 nucleotides wherein, sequentially, 25 nucleotides are complementarity to a region of a target RNA, 4 nucleotides form a bulge, and 25 nucleotides are complementarity to the region of the target RNA. As another example, a targeting sequence comprises a total of 118 nucleotides wherein, sequentially, 25 nucleotides are complementarity to a region of a target RNA, 4 nucleotides form a bulge, 25 nucleotides are complementarity to the region of the target RNA, 14 nucleotides form a loop, and 50 nucleotides are complementary to the region of the target RNA.

In some cases, a subject engineered polynucleotide is configured to facilitate editing of a base of a nucleotide of a polynucleotide of a region of a subject target RNA, to modulate expression of a polypeptide encoded by the subject target RNA, or both. In order to facilitate editing, an engineered polynucleotide of the disclosure may recruit an RNA editing entity. In certain embodiments, an engineered polynucleotide comprises an RNA editing entity recruiting domain. In certain embodiments, an engineered polynucleotide lacks an RNA editing entity recruiting domain. Either way, a subject engineered polynucleotide can be capable of binding an RNA editing entity, or be bound by it, and facilitate editing of a subject target RNA.

In an aspect, a subject engineered polynucleotide comprises an RNA editing entity recruiting domain. An RNA editing entity can be recruited by an RNA editing entity recruiting domain on an engineered polynucleotide. In some cases, an engineered polynucleotide can be configured to facilitate an editing of a base of a nucleotide or polynucleotide of a region of an RNA by a subject RNA editing entity.

Various RNA editing entity recruiting domains can be utilized. In an embodiment, a recruiting domain comprises: Glutamate ionotropic receptor AMPA type subunit 2 (GluR2), APOBEC, MS2-bacteriophage-coat-protein-recruiting domain, Alu, a TALEN recruiting domain, a Zn-finger polypeptide recruiting domain, a mega-TAL recruiting domain, or a Cas13 recruiting domain, combinations thereof, or modified versions thereof. In certain embodiments, more than one recruiting domain can be included in an engineered polynucleotide of the disclosure. In cases where a recruiting sequence is present, the recruiting sequence can be utilized to position the RNA editing entity to effectively react with a subject target RNA after the targeting sequence, for example an antisense sequence, hybridizes to a region of the target RNA. In some cases, a recruiting sequence can allow for transient binding of the RNA editing entity to the engineered polynucleotide. In other cases, the recruiting sequence allows for permanent binding of the RNA editing entity to the polynucleotide. A recruiting sequence can be of any length. In some cases, a recruiting sequence is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides in length. In some cases, a recruiting sequence is about 45 nucleotides in length. In some cases, at least a portion of a recruiting sequence comprises from at least 1 to about 75 nucleotides. In some cases, at least a portion of a recruiting sequence comprises from about 45 nucleotides to about 60 nucleotides. In some cases, at least a portion of a recruiting sequence comprises from at least 1 to about 500 nucleotides.

In an embodiment, an RNA editing entity recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example, for enhanced recruitment. In some embodiments, a GluR2 sequence can comprise a portion of a naturally occurring GluR2 sequence and a synthetic sequence.

In an embodiment, a recruiting domain comprises a GluR2 sequence, or a sequence having at least about 80%, 85%, 90%, 95%, 98%, 99%, or 100% identity to: GUGGAAUAGUAUAACAAUAUGCUAAAUGUUGUUAUAGUAUCCCAC (SEQ ID NO: 1). In some cases, a recruiting domain can comprise at least about 80%, 85%, 90%, 95%, 99%, or 100% sequence homology to at least about 10, 15, 20, 25, or 30 nucleotides of SEQ ID NO: 1. In some embodiments, a recruiting domain can comprise at least about 90%, 95%, 96%, 97%, 98%, or 99% sequence homology to SEQ ID NO: 1.

Additional RNA editing entity recruiting domains are also contemplated. In an embodiment, a recruiting domain comprises an apolipoprotein B mRNA editing enzyme, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In another embodiment, a recruiting domain can be from an MS2-bacteriophage-coat-protein-recruiting domain. In another embodiment, a recruiting domain can be from an Alu domain. In some cases, a recruiting domain can comprise at least about: 80%, 85%, 90%, 95%, 99%, or 100% sequence homology to at least about: 15, 20, 25, 30, or 35 nucleotides of an APOBEC, MS2-bacteriophage-coat-protein-recruiting domain, or Alu domain.

In some embodiments, a recruiting domain comprises a CRISPR associated recruiting domain sequence. For example, a CRISPR associated recruiting sequence can comprise a Cas protein sequence. In some cases, a Cas13 recruiting domain can comprise a Cas13a recruiting domain, a Cas13b recruiting domain, a Cas13c recruiting domain, or a Cas13d recruiting domain. In some cases, an RNA editing entity recruiting domain can comprise at least about 80% sequence homology to at least about 20 nucleic acids of a Cas13b recruiting domain. In some embodiments, an RNA editing entity recruiting domain can comprise at least about 80%, 85%, 90%, 95%, 99%, or 100% sequence homology to a Cas13b recruiting domain. In some cases, an RNA editing entity recruiting domain can comprise at least about: 80%, 85%, 90%, 95%, 99%, or 100% sequence homology to at least about: 15, 20, 25, 30, or 35 nucleic acids of a Cas13b domain. In some embodiments, at least a portion of an RNA editing entity recruiting domain can comprise at least about 80%85%, 90%, 95%, 99%, or 100% sequence homology to a Cas13b domain encoding sequence. In some cases, at least a portion of an RNA editing entity recruiting domain can comprise at least about 85% sequence homology to a Cas13b domain encoding sequence. In some embodiments, at least a portion of an RNA editing entity recruiting domain can comprise at least about 90% sequence homology to a Cas13b domain encoding sequence. In some cases, at least a portion of an RNA editing entity recruiting domain can comprise at least about 95% sequence homology to a Cas13b domain encoding sequence. In some cases, at least a portion of an RNA editing entity recruiting domain can comprise at least about 99% sequence homology to a Cas13b domain encoding sequence. In some cases, at least a portion of an RNA editing entity recruiting domain can comprise at least about 100% sequence homology to a Cas13b domain encoding sequence. In some embodiments, a Cas13b-domain-encoding sequence can be a non-naturally occurring sequence. In some cases, a Cas13b-domain-encoding sequence can comprise a modified portion. In some embodiments, a Cas13b-domain-encoding sequence can comprise a portion of a naturally occurring Cas13b-domain-encoding-sequence.

Any number of recruiting sequences may be found in a polynucleotide of the present disclosure. In some cases, at least about 1, 2, 3, 4, 5, 6, 7, 8, 9, or up to about 10 recruiting sequences are included in a polynucleotide. Recruiting sequences may be located at any position of subject polynucleotides. In some cases, a recruiting sequence is on an N-terminus, middle, or C-terminus of a polynucleotide. A recruiting sequence can be upstream or downstream of a targeting sequence. In some cases, a recruiting sequence flanks a targeting sequence of a subject polynucleotide. A recruiting sequence can comprise all ribonucleotides or deoxyribonucleotides, although a recruiting sequence comprising both ribo- and deoxyribonucleotides is not excluded.

In some cases, an engineered polynucleotide can comprise recruiting domain, and one or more structural features or a structured motif. Structural features can comprise any one of a: mismatch, symmetrical bulge, asymmetrical bulge, symmetrical internal loop, asymmetrical internal loop, hairpins, wobble base pairs, chemical modification, or any combination thereof. In an aspect, a double stranded RNA (dsRNA) substrate, for example hybridized polynucleotide strands, can be formed upon hybridization of an engineered polynucleotide of the present disclosure to a region of a target RNA. Described herein can be a feature, which corresponds to one of several structural features that can be present in a dsRNA substrate of the present disclosure. Examples of features include a mismatch, a bulge (symmetrical bulge or asymmetrical bulge), an internal loop (symmetrical internal loop or asymmetrical internal loop), or a hairpin (e.g. a non-targeting domain). Engineered polynucleotides of the present disclosure can have from 1 to 50 features and a recruiting domain. Engineered polynucleotides of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features and a recruiting domain.

In cases where a recruiting domain can be absent, an engineered polynucleotide can still be capable of associating with a subject RNA editing entity (e.g., ADAR) to facilitate editing of a target RNA and/or modulate expression of a polypeptide encoded by a subject target RNA. This can be achieved through one or more structural feature or a structured motif. Structural features can comprise any one of a: mismatch, symmetrical bulge, asymmetrical bulge, symmetrical internal loop, asymmetrical internal loop, hairpins, wobble base pairs, chemical modification, or any combination thereof. In an aspect, a double stranded RNA (dsRNA) substrate, for example hybridized polynucleotide strands, can be formed upon hybridization of an engineered polynucleotide of the present disclosure to a region of a target RNA. Described herein can be a feature, which corresponds to one of several structural features that can be present in a dsRNA substrate of the present disclosure. Examples of features include a mismatch, a bulge (symmetrical bulge or asymmetrical bulge), an internal loop (symmetrical internal loop or asymmetrical internal loop), or a hairpin (a hairpin comprising a non-targeting domain). Engineered polynucleotides of the present disclosure can have from 1 to 50 features. Engineered polynucleotides of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features.

As disclosed herein, a structured motif comprises two or more features in a dsRNA substrate.

A double stranded RNA (dsRNA) substrate can be formed upon hybridization of an engineered polynucleotide of the present disclosure to a target RNA (e.g., a region of the target RNA). As disclosed herein, a mismatch refers to a nucleotide in a polynucleotide that can be unpaired to an opposing nucleotide in a target RNA within the dsRNA. A mismatch can comprise any two nucleotides that do not base pair, are not complementary, or both. In some embodiments, a mismatch can be an A/C mismatch. An A/C mismatch can comprise a C in an engineered polynucleotide of the present disclosure opposite an A in a target RNA (e.g., in a region of the target RNA). In an embodiment, a mismatch comprises an A/C mismatch, wherein the A can be in the target RNA and the C can be in the targeting sequence of the engineered polynucleotide. In another embodiment, the A in the A/C mismatch can be the base of the nucleotide in the target RNA edited by a subject RNA editing entity. In another embodiment, the A in the A/C mismatch can be the base of the nucleotide in the region of the target RNA edited by a subject RNA editing entity. An A/C mismatch can comprise a A in an engineered polynucleotide of the present disclosure opposite an C in a target RNA (e.g., in a region of the target RNA). In an embodiment, a mismatch comprises a G/G mismatch. In an embodiment, a G/G mismatch can comprise a G in an engineered polynucleotide of the present disclosure opposite a G in a target RNA. In some embodiments, a mismatch positioned 5′ of the edit site can facilitate base-flipping of the target A to be edited. A mismatch can also help confer sequence specificity.

In an aspect, a structural feature can form in an engineered polynucleotide independently of hybridization to a region of a target RNA. In other cases, a structural feature can form when an engineered polynucleotide binds to a region of a target RNA. A structural feature can also form when an engineered polynucleotide associates with other molecules such as a peptide, a nucleotide, or a small molecule. In certain embodiments, a structural feature of an engineered polynucleotide can be formed independent of hybridization to a region of a target RNA, and its structure can change as a result of the engineered polynucleotide hybridization to a target RNA region. In certain embodiments, a structural feature can be present when an engineered polynucleotide can be in association with a target RNA.

In some cases, a structural feature can be a hairpin. In some cases, an engineered polynucleotide can lack a hairpin domain. In other cases, an engineered polynucleotide can comprise a hairpin domain or more than one hairpin domain. A hairpin can be located anywhere in an engineered polynucleotide. As disclosed herein, a hairpin can be an RNA duplex wherein a single RNA strand has folded in upon itself to form the RNA duplex. The single RNA strand folds upon itself due to having nucleotide sequences upstream and downstream of the folding region base pairs to each other. A hairpin can have from 10 to 500 nucleotides in length of the entire duplex structure. The stem-loop structure of a hairpin can be from 3 to 15 nucleotides long. A hairpin can be present in any of the engineered polynucleotides disclosed herein. The engineered polynucleotides disclosed herein can comprise from 1 to 10 hairpins. In some embodiments, the engineered polynucleotides disclosed herein comprise 1 hairpin. In some embodiments, the engineered polynucleotides disclosed herein comprise 2 hairpins. As disclosed herein, a hairpin can refer to a recruitment hairpin or a hairpin or a non-recruitment hairpin. A hairpin can be located anywhere within the engineered polynucleotides of the present disclosure. In some embodiments, one or more hairpins can be present at the 3′ end of an engineered polynucleotide of the present disclosure, at the 5′ end of an engineered polynucleotide of the present disclosure or within the targeting sequence of an engineered polynucleotide of the present disclosure, or any combination thereof.

A recruitment hairpin can recruit an RNA editing entity, such as ADAR. In some embodiments, a recruitment hairpin comprises a GluR2 domain. In some embodiments, a recruitment hairpin comprises an Alu domain.

In yet another aspect, a structural feature comprises a non-recruitment hairpin. A non-recruitment hairpin, as disclosed herein, can exhibit functionality that improves localization of the engineered polynucleotide to the target RNA. In some embodiments, a non-recruitment hairpin exhibits functionality that improves localization of the engineered polynucleotide to the region of the target RNA for hybridization. In some embodiments, the non-recruitment hairpin improves nuclear retention. In some embodiments, the non-recruitment hairpin comprises a hairpin from U7 snRNA.

In another aspect, a structural feature comprises a wobble base. A wobble base pair refers to two bases that weakly pair. For example, a wobble base pair of the present disclosure can refer to a G paired with a U.

A hairpin of the present disclosure can be of any length. In an aspect, a hairpin can be from about 5-200 or more nucleotides. In some cases, a hairpin can comprise about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 or more nucleotides. In other cases, a hairpin can also comprise from 5 to 10, 5 to 20, 5 to 30, 5 to 40, 5 to 50, 5 to 60, 5 to 70, 5 to 80, 5 to 90, 5 to 100, 5 to 110, 5 to 120, 5 to 130, 5 to 140, 5 to 150, 5 to 160, 5 to 170, 5 to 180, 5 to 190, 5 to 200, 5 to 210, 5 to 220, 5 to 230, 5 to 240, 5 to 250, 5 to 260, 5 to 270, 5 to 280, 5 to 290, 5 to 300, 5 to 310, 5 to 320, 5 to 330, 5 to 340, 5 to 350, 5 to 360, 5 to 370, 5 to 380, 5 to 390, or 5 to 400 nucleotides. A hairpin can be a structural feature formed from a single strand of RNA with sufficient complementarity to itself to hybridize into a double stranded RNA motif/structure consisting of double-stranded hybridized RNA separated by a nucleotide loop.

In some cases, a structural feature can be a bulge. A bulge can comprise a single (intentional) nucleic acid mismatch between the target strand and an engineered polynucleotide strand. In other cases, more than one consecutive mismatch between strands constitutes a bulge as long as the bulge region, mismatched stretch of bases, can be flanked on both sides with hybridized, complementary dsRNA regions. A bulge can be located at any location of a polynucleotide. In some cases, a bulge can be located from about 30 to about 70 nucleotides from a 5′ hydroxyl or the 3′ hydroxyl.

In an embodiment, a double stranded RNA (dsRNA) substrate can be formed upon hybridization of an engineered polynucleotide of the present disclosure to a target RNA. As disclosed herein, a bulge refers to the structure formed upon formation of the dsRNA substrate, where nucleotides in either the engineered polynucleotide or the target RNA can be not complementary to their positional counterparts on the opposite strand. A bulge can change the secondary or tertiary structure of the dsRNA substrate. A bulge can have from 1 to 4 nucleotides on the engineered polynucleotide side of the dsRNA substrate or the target RNA side of the dsRNA substrate. In some embodiments, the engineered polynucleotides of the present disclosure have 2 bulges. In some embodiments, the engineered polynucleotides of the present disclosure have 3 bulges. In some embodiments, the engineered polynucleotides of the present disclosure have 4 bulges. In some embodiments, the presence of a bulge in a dsRNA substrate can position ADAR to selectively edit the target A in the target RNA and reduce off-target editing of non-targets. In some embodiments, the presence of a bulge in a dsRNA substrate can recruit additional ADAR. Bulges in dsRNA substrates disclosed herein can recruit other proteins, such as other RNA editing entities. In some embodiments, a bulge positioned 5′ of the edit site can facilitate base-flipping of the target A to be edited. A bulge can also help confer sequence specificity. A bulge can help direct ADAR editing by constraining it in an orientation that yield selective editing of the target A. In some embodiments, selective editing of the target A is achieved by positioning the target A between two bulges (e.g., positioned between a 5′ end bulge and a 3′ end bulge, based on the engineered polynucleotide). In some embodiments, the two bulges are both symmetrical bulges. In some embodiments, the two bulges each are formed by 2 nucleotides on the engineered polynucleotide side of the dsRNA target and 2 nucleotides on the target RNA side of the dsRNA substrate. In some embodiments, the two bulges each are formed by 3 nucleotides on the engineered polynucleotide side of the dsRNA target and 3 nucleotides on the target RNA side of the dsRNA substrate. In some embodiments, the two bulges each are formed by 4 nucleotides on the engineered polynucleotide side of the dsRNA target and 4 nucleotides on the target RNA side of the dsRNA substrate. In some embodiments, the target A is position between the two bulges, and is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides from a bulge (e.g., from a 5′end bulge or a 3′ end bulge). In some embodiments, additional structural features are located between the bulges (e.g., between the 5′end bulge and the 3′end bulge). In some embodiments, a mismatch in a bulge comprises a nucleotide base for editing in the target RNA (e.g., an A/C mismatch in the bulge, wherein part of the bulge in the engineered polynucleotide comprises a C mismatched to an A in the part of the bulge in the target RNA, and the A is edited).

In an aspect, a double stranded RNA (dsRNA) substrate can be formed upon hybridization of an engineered polynucleotide of the present disclosure to a target RNA. A bulge can be a symmetrical bulge or an asymmetrical bulge. A bulge can be formed by 1 to 4 participating nucleotides on either the polynucleotide side or the target RNA side of the dsRNA substrate. A symmetrical bulge can be formed when the same number of nucleotides can be present on each side of the bulge. A symmetrical bulge can have from 2 to 4 nucleotides on the engineered polynucleotide side of the dsRNA substrate or the target RNA side of the dsRNA substrate. For example, a symmetrical bulge in a dsRNA substrate of the present disclosure can have the same number of nucleotides on the engineered polynucleotide side and the target RNA side of the dsRNA substrate. A symmetrical bulge of the present disclosure can be formed by 2 nucleotides on the engineered polynucleotide side of the dsRNA target and 2 nucleotides on the target RNA side of the dsRNA substrate. A symmetrical bulge of the present disclosure can be formed by 3 nucleotides on the engineered polynucleotide side of the dsRNA target and 3 nucleotides on the target RNA side of the dsRNA substrate. A symmetrical bulge of the present disclosure can be formed by 4 nucleotides on the engineered polynucleotide side of the dsRNA target and 4 nucleotides on the target RNA side of the dsRNA substrate.

A double stranded RNA (dsRNA) substrate can be formed upon hybridization of an engineered polynucleotide of the present disclosure to a target RNA. A bulge can be a symmetrical bulge or an asymmetrical bulge. An asymmetrical bulge can be formed when a different number of nucleotides can be present on each side of the bulge. An asymmetrical bulge can have from 1 to 4 participating nucleotides on either the polynucleotide side or the target RNA side of the dsRNA substrate. For example, an asymmetrical bulge in a dsRNA substrate of the present disclosure can have different numbers of nucleotides on the engineered polynucleotide side and the target RNA side of the dsRNA substrate. An asymmetrical bulge of the present disclosure can be formed by 0 nucleotides on the engineered polynucleotide side of the dsRNA substrate and 1 nucleotide on the target RNA side of the dsRNA substrate. An asymmetrical bulge of the present disclosure can be formed by 0 nucleotides on the target RNA side of the dsRNA substrate and 1 nucleotide on the engineered polynucleotide side of the dsRNA substrate. An asymmetrical bulge of the present disclosure can be formed by 0 nucleotides on the engineered polynucleotide side of the dsRNA substrate and 2 nucleotides on the target RNA side of the dsRNA substrate. An asymmetrical bulge of the present disclosure can be formed by 0 nucleotides on the target RNA side of the dsRNA substrate and 2 nucleotides on the engineered polynucleotide side of the dsRNA substrate. An asymmetrical bulge of the present disclosure can be formed by 0 nucleotides on the engineered polynucleotide side of the dsRNA substrate and 3 nucleotides on the target RNA side of the dsRNA substrate. An asymmetrical bulge of the present disclosure can be formed by 0 nucleotides on the target RNA side of the dsRNA substrate and 3 nucleotides on the engineered polynucleotide side of the dsRNA substrate. An asymmetrical bulge of the present disclosure can be formed by 0 nucleotides on the engineered polynucleotide side of the dsRNA substrate and 4 nucleotides on the target RNA side of the dsRNA substrate. An asymmetrical bulge of the present disclosure can be formed by 0 nucleotides on the target RNA side of the dsRNA substrate and 4 nucleotides on the engineered polynucleotide side of the dsRNA substrate. An asymmetrical bulge of the present disclosure can be formed by 1 nucleotide on the engineered polynucleotide side of the dsRNA substrate and 2 nucleotides on the target RNA side of the dsRNA substrate. An asymmetrical bulge of the present disclosure can be formed by 1 nucleotide on the target RNA side of the dsRNA substrate and 2 nucleotides on the engineered polynucleotide side of the dsRNA substrate. An asymmetrical bulge of the present disclosure can be formed by 1 nucleotide on the engineered polynucleotide side of the dsRNA substrate and 3 nucleotides on the target RNA side of the dsRNA substrate. An asymmetrical bulge of the present disclosure can be formed by 1 nucleotide on the target RNA side of the dsRNA substrate and 3 nucleotides on the engineered polynucleotide side of the dsRNA substrate. An asymmetrical bulge of the present disclosure can be formed by 1 nucleotides on the engineered polynucleotide side of the dsRNA substrate and 4 nucleotides on the target RNA side of the dsRNA substrate. An asymmetrical bulge of the present disclosure can be formed by 1 nucleotide on the target RNA side of the dsRNA substrate and 4 nucleotides on the engineered polynucleotide side of the dsRNA substrate. An asymmetrical bulge of the present disclosure can be formed by 2 nucleotides on the engineered polynucleotide side of the dsRNA substrate and 3 nucleotides on the target RNA side of the dsRNA substrate. An asymmetrical bulge of the present disclosure can be formed by 2 nucleotides on the target RNA side of the dsRNA substrate and 3 nucleotides on the engineered polynucleotide side of the dsRNA substrate. An asymmetrical bulge of the present disclosure can be formed by 2 nucleotides on the engineered polynucleotide side of the dsRNA substrate and 4 nucleotides on the target RNA side of the dsRNA substrate. An asymmetrical bulge of the present disclosure can be formed by 2 nucleotides on the target RNA side of the dsRNA substrate and 4 nucleotides on the engineered polynucleotide side of the dsRNA substrate. An asymmetrical bulge of the present disclosure can be formed by 3 nucleotides on the engineered polynucleotide side of the dsRNA substrate and 4 nucleotides on the target RNA side of the dsRNA substrate. An asymmetrical bulge of the present disclosure can be formed by 3 nucleotides on the target RNA side of the dsRNA substrate and 4 nucleotides on the engineered polynucleotide side of the dsRNA substrate. In some embodiments, an asymmetrical bulge increases efficiency of editing a target A. In some embodiments, an asymmetrical bulge that increases efficiency of editing a target A is an asymmetrical bulge that is formed to reduce the number of adenosines in the sequence of the engineered polynucleotide. Non-limiting examples of an asymmetrical bulge that increases efficiency of editing a target A are an asymmetrical bulge formed by 0 nucleotides on the engineered polynucleotide side of the dsRNA substrate and 1 nucleotide on the target RNA side of the dsRNA substrate; an asymmetrical bulge of formed by 0 nucleotides on the engineered polynucleotide side of the dsRNA substrate and 2 nucleotides on the target RNA side of the dsRNA substrate; an asymmetrical bulge of formed by 0 nucleotides on the engineered polynucleotide side of the dsRNA substrate and 3 nucleotides on the target RNA side of the dsRNA substrate; an asymmetrical bulge of formed by 0 nucleotides on the engineered polynucleotide side of the dsRNA substrate and 4 nucleotides on the target RNA side of the dsRNA substrate; an asymmetrical bulge of formed by 1 nucleotide on the engineered polynucleotide side of the dsRNA substrate and 2 nucleotides on the target RNA side of the dsRNA substrate; an asymmetrical bulge of formed by 1 nucleotide on the engineered polynucleotide side of the dsRNA substrate and 3 nucleotides on the target RNA side of the dsRNA substrate; an asymmetrical bulge of formed by 1 nucleotide on the engineered polynucleotide side of the dsRNA substrate and 4 nucleotides on the target RNA side of the dsRNA substrate; an asymmetrical bulge of formed by 2 nucleotides on the engineered polynucleotide side of the dsRNA substrate and 3 nucleotides on the target RNA side of the dsRNA substrate; an asymmetrical bulge of formed by 2 nucleotides on the engineered polynucleotide side of the dsRNA substrate and 4 nucleotides on the target RNA side of the dsRNA substrate; and an asymmetrical bulge of formed by 3 nucleotides on the engineered polynucleotide side of the dsRNA substrate and 4 nucleotides on the target RNA side of the dsRNA substrate.

In an aspect, a double stranded RNA (dsRNA) substrate can be formed upon hybridization of an engineered polynucleotide of the present disclosure to a target RNA. In some cases, a structural feature can be a loop. In some embodiments, the loop is an internal loop. As disclosed herein, an internal loop refers to the structure formed upon formation of the dsRNA substrate, where nucleotides in either the engineered polynucleotide or the target RNA can be not complementary to their positional counterparts on the opposite strand and where one side of the internal loop, either on the target RNA side or the engineered polynucleotide side of the dsRNA substrate, has greater than 5 nucleotides. An internal loop can be a symmetrical internal loop or an asymmetrical internal loop. Internal loops present in the vicinity of the edit site can help with base flipping of the target A in the target RNA to be edited. A double stranded RNA (dsRNA) substrate can be formed upon hybridization of an engineered polynucleotide of the present disclosure to a target RNA. An internal loop can be a symmetrical internal loop or an asymmetrical internal loop. In some embodiments, selective editing of the target A is achieved by positioning the target A between two loops (e.g., positioned between a 5′ end loop and a 3′ end loop, based on the engineered polynucleotide). In some embodiments, the two loops are both symmetrical loops. In some embodiments, the two loops each are formed by 5 nucleotides on the engineered polynucleotide side of the dsRNA target and 5 nucleotides on the target RNA side of the dsRNA substrate. In some embodiments, the two loops each are formed by 6 nucleotides on the engineered polynucleotide side of the dsRNA target and 6 nucleotides on the target RNA side of the dsRNA substrate. In some embodiments, the two loops each are formed by 7 nucleotides on the engineered polynucleotide side of the dsRNA target and 7 nucleotides on the target RNA side of the dsRNA substrate. In some embodiments, the two loops each are formed by 8 nucleotides on the engineered polynucleotide side of the dsRNA target and 8 nucleotides on the target RNA side of the dsRNA substrate. In some embodiments, the two loops each are formed by 9 nucleotides on the engineered polynucleotide side of the dsRNA target and 9 nucleotides on the target RNA side of the dsRNA substrate. In some embodiments, the two loops each are formed by 10 nucleotides on the engineered polynucleotide side of the dsRNA target and 10 nucleotides on the target RNA side of the dsRNA substrate. In some embodiments, the target A is position between the two loops, and is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides from a loop (e.g., from a 5′end loop or a 3′ end loop). In some embodiments, additional structural features are located between the loops (e.g., between the 5′end loop and the 3′end loop). In some embodiments, a mismatch in a loop comprises a nucleotide base for editing in the target RNA (e.g., an A/C mismatch in the loop, wherein part of the bulge in the engineered polynucleotide comprises a C mismatched to an A in the part of the loop in the target RNA, and the A is edited).

A symmetrical internal loop can be formed when the same number of nucleotides can be present on each side of the internal loop. For example, a symmetrical internal loop in a dsRNA substrate of the present disclosure can have the same number of nucleotides on the engineered polynucleotide side and the target RNA side of the dsRNA substrate. A symmetrical internal loop of the present disclosure can be formed by 5 nucleotides on the engineered polynucleotide side of the dsRNA target and 5 nucleotides on the target RNA side of the dsRNA substrate. A symmetrical internal loop of the present disclosure can be formed by 6 nucleotides on the engineered polynucleotide side of the dsRNA target and 6 nucleotides on the target RNA side of the dsRNA substrate. A symmetrical internal loop of the present disclosure can be formed by 7 nucleotides on the engineered polynucleotide side of the dsRNA target and 7 nucleotides on the target RNA side of the dsRNA substrate. A symmetrical internal loop of the present disclosure can be formed by 8 nucleotides on the engineered polynucleotide side of the dsRNA target and 8 nucleotides on the target RNA side of the dsRNA substrate. A symmetrical internal loop of the present disclosure can be formed by 9 nucleotides on the engineered polynucleotide side of the dsRNA target and 9 nucleotides on the target RNA side of the dsRNA substrate. A symmetrical internal loop of the present disclosure can be formed by 10 nucleotides on the engineered polynucleotide side of the dsRNA target and 10 nucleotides on the target RNA side of the dsRNA substrate.

In an aspect, a double stranded RNA (dsRNA) substrate can be formed upon hybridization of an engineered polynucleotide of the present disclosure to a target RNA. As disclosed herein, an internal loop refers to the structure formed upon formation of the dsRNA substrate, where nucleotides in either the engineered polynucleotide or the target RNA are not complementary to their positional counterparts on the opposite strand and where one side of the internal loop, either on the target RNA side or the engineered polynucleotide side of the dsRNA substrate, has greater than 5 nucleotides. An internal loop may be a symmetrical internal loop or an asymmetrical internal loop. Internal loops present in the vicinity of the edit site may help with base flipping of the target A in the target RNA to be edited. A double stranded RNA (dsRNA) substrate is formed upon hybridization of an engineered polynucleotide of the present disclosure to a target RNA. An internal loop may be a symmetrical internal loop or an asymmetrical internal loop. A symmetrical internal loop is formed when the same number of nucleotides is present on each side of the internal loop. For example, a symmetrical internal loop in a dsRNA substrate of the present disclosure may have the same number of nucleotides on the engineered polynucleotide side and the target RNA side of the dsRNA substrate. A symmetrical internal loop of the present disclosure may be formed by 5 nucleotides on the engineered polynucleotide side of the dsRNA target and 5 nucleotides on the target RNA side of the dsRNA substrate. A symmetrical internal loop of the present disclosure may be formed by 6 nucleotides on the engineered polynucleotide side of the dsRNA target and 6 nucleotides on the target RNA side of the dsRNA substrate. A symmetrical internal loop of the present disclosure may be formed by 7 nucleotides on the engineered polynucleotide side of the dsRNA target and 7 nucleotides on the target RNA side of the dsRNA substrate. A symmetrical internal loop of the present disclosure may be formed by 8 nucleotides on the engineered polynucleotide side of the dsRNA target and 8 nucleotides on the target RNA side of the dsRNA substrate. A symmetrical internal loop of the present disclosure may be formed by 9 nucleotides on the engineered polynucleotide side of the dsRNA target and 9 nucleotides on the target RNA side of the dsRNA substrate. A symmetrical internal loop of the present disclosure may be formed by 10 nucleotides on the engineered polynucleotide side of the dsRNA target and 10 nucleotides on the target RNA side of the dsRNA substrate. One side of the internal loop, either on the target RNA side or the engineered polynucleotide side of the dsRNA substrate, may be formed by from 5 to 150 nucleotides. One side of the internal loop may be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop may be formed by 5 nucleotides. One side of the internal loop may be formed by 10 nucleotides. One side of the internal loop may be formed by 15 nucleotides. One side of the internal loop may be formed by 20 nucleotides. One side of the internal loop may be formed by 25 nucleotides. One side of the internal loop may be formed by 30 nucleotides. One side of the internal loop may be formed by 35 nucleotides. One side of the internal loop may be formed by 40 nucleotides. One side of the internal loop may be formed by 45 nucleotides. One side of the internal loop may be formed by 50 nucleotides. One side of the internal loop may be formed by 55 nucleotides. One side of the internal loop may be formed by 60 nucleotides. One side of the internal loop may be formed by 65 nucleotides. One side of the internal loop may be formed by 70 nucleotides. One side of the internal loop may be formed by 75 nucleotides. One side of the internal loop may be formed by 80 nucleotides. One side of the internal loop may be formed by 85 nucleotides. One side of the internal loop may be formed by 90 nucleotides. One side of the internal loop may be formed by 95 nucleotides. One side of the internal loop may be formed by 100 nucleotides. One side of the internal loop may be formed by 110 nucleotides. One side of the internal loop may be formed by 120 nucleotides. One side of the internal loop may be formed by 130 nucleotides. One side of the internal loop may be formed by 140 nucleotides. One side of the internal loop may be formed by 150 nucleotides. One side of the internal loop may be formed by 200 nucleotides. One side of the internal loop may be formed by 250 nucleotides. One side of the internal loop may be formed by 300 nucleotides. One side of the internal loop may be formed by 350 nucleotides. One side of the internal loop may be formed by 400 nucleotides. One side of the internal loop may be formed by 450 nucleotides. One side of the internal loop may be formed by 500 nucleotides. One side of the internal loop may be formed by 600 nucleotides. One side of the internal loop may be formed by 700 nucleotides. One side of the internal loop may be formed by 800 nucleotides. One side of the internal loop may be formed by 900 nucleotides. One side of the internal loop may be formed by 1000 nucleotides. An internal loop may be a symmetrical internal loop or an asymmetrical internal loop. Internal loops present in the vicinity of the edit site may help with base flipping of the target A in the target RNA to be edited. A double stranded RNA (dsRNA) substrate is formed upon hybridization of an engineered polynucleotide of the present disclosure to a target RNA. An internal loop may be a symmetrical internal loop or an asymmetrical internal loop. A symmetrical internal loop is formed when the same number of nucleotides is present on each side of the internal loop. For example, a symmetrical internal loop in a dsRNA substrate of the present disclosure may have the same number of nucleotides on the engineered polynucleotide side and the target RNA side of the dsRNA substrate. A symmetrical internal loop of the present disclosure may be formed by from 5 to 150 nucleotides on the engineered polynucleotide side of the dsRNA target and from 5 to 150 nucleotides on the target RNA side of the dsRNA substrate, wherein the number of nucleotides is the same on the engineered side of the dsRNA target and the target RNA side of the dsRNA substrate. A symmetrical internal loop of the present disclosure may be formed by from 5 to 1000 nucleotides on the engineered polynucleotide side of the dsRNA target and from 5 to 1000 nucleotides on the target RNA side of the dsRNA substrate, wherein the number of nucleotides is the same on the engineered side of the dsRNA target and the target RNA side of the dsRNA substrate. A symmetrical internal loop of the present disclosure may be formed by 5 nucleotides on the engineered polynucleotide side of the dsRNA target and 5 nucleotides on the target RNA side of the dsRNA substrate. A symmetrical internal loop of the present disclosure may be formed by 6 nucleotides on the engineered polynucleotide side of the dsRNA target and 6 nucleotides on the target RNA side of the dsRNA substrate. A symmetrical internal loop of the present disclosure may be formed by 7 nucleotides on the engineered polynucleotide side of the dsRNA target and 7 nucleotides on the target RNA side of the dsRNA substrate. A symmetrical internal loop of the present disclosure may be formed by 8 nucleotides on the engineered polynucleotide side of the dsRNA target and 8 nucleotides on the target RNA side of the dsRNA substrate. A symmetrical internal loop of the present disclosure may be formed by 9 nucleotides on the engineered polynucleotide side of the dsRNA target and 9 nucleotides on the target RNA side of the dsRNA substrate. A symmetrical internal loop of the present disclosure may be formed by 10 nucleotides on the engineered polynucleotide side of the dsRNA target and 10 nucleotides on the target RNA side of the dsRNA substrate. A symmetrical internal loop of the present disclosure may be formed by 15 nucleotides on the engineered polynucleotide side of the dsRNA target and 15 nucleotides on the target RNA side of the dsRNA substrate. A symmetrical internal loop of the present disclosure may be formed by 20 nucleotides on the engineered polynucleotide side of the dsRNA target and 20 nucleotides on the target RNA side of the dsRNA substrate. A symmetrical internal loop of the present disclosure may be formed by 30 nucleotides on the engineered polynucleotide side of the dsRNA target and 30 nucleotides on the target RNA side of the dsRNA substrate. A symmetrical internal loop of the present disclosure may be formed by 40 nucleotides on the engineered polynucleotide side of the dsRNA target and 40 nucleotides on the target RNA side of the dsRNA substrate. A symmetrical internal loop of the present disclosure may be formed by 50 nucleotides on the engineered polynucleotide side of the dsRNA target and 50 nucleotides on the target RNA side of the dsRNA substrate. A symmetrical internal loop of the present disclosure may be formed by 60 nucleotides on the engineered polynucleotide side of the dsRNA target and 60 nucleotides on the target RNA side of the dsRNA substrate. A symmetrical internal loop of the present disclosure may be formed by 70 nucleotides on the engineered polynucleotide side of the dsRNA target and 70 nucleotides on the target RNA side of the dsRNA substrate. A symmetrical internal loop of the present disclosure may be formed by 80 nucleotides on the engineered polynucleotide side of the dsRNA target and 80 nucleotides on the target RNA side of the dsRNA substrate. A symmetrical internal loop of the present disclosure may be formed by 90 nucleotides on the engineered polynucleotide side of the dsRNA target and 90 nucleotides on the target RNA side of the dsRNA substrate. A symmetrical internal loop of the present disclosure may be formed by 100 nucleotides on the engineered polynucleotide side of the dsRNA target and 100 nucleotides on the target RNA side of the dsRNA substrate. A symmetrical internal loop of the present disclosure may be formed by 110 nucleotides on the engineered polynucleotide side of the dsRNA target and 110 nucleotides on the target RNA side of the dsRNA substrate. A symmetrical internal loop of the present disclosure may be formed by 120 nucleotides on the engineered polynucleotide side of the dsRNA target and 120 nucleotides on the target RNA side of the dsRNA substrate. A symmetrical internal loop of the present disclosure may be formed by 130 nucleotides on the engineered polynucleotide side of the dsRNA target and 130 nucleotides on the target RNA side of the dsRNA substrate. A symmetrical internal loop of the present disclosure may be formed by 140 nucleotides on the engineered polynucleotide side of the dsRNA target and 140 nucleotides on the target RNA side of the dsRNA substrate. A symmetrical internal loop of the present disclosure may be formed by 150 nucleotides on the engineered polynucleotide side of the dsRNA target and 150 nucleotides on the target RNA side of the dsRNA substrate. A symmetrical internal loop of the present disclosure may be formed by 200 nucleotides on the engineered polynucleotide side of the dsRNA target and 200 nucleotides on the target RNA side of the dsRNA substrate. A symmetrical internal loop of the present disclosure may be formed by 250 nucleotides on the engineered polynucleotide side of the dsRNA target and 250 nucleotides on the target RNA side of the dsRNA substrate. A symmetrical internal loop of the present disclosure may be formed by 300 nucleotides on the engineered polynucleotide side of the dsRNA target and 300 nucleotides on the target RNA side of the dsRNA substrate. A symmetrical internal loop of the present disclosure may be formed by 350 nucleotides on the engineered polynucleotide side of the dsRNA target and 350 nucleotides on the target RNA side of the dsRNA substrate. A symmetrical internal loop of the present disclosure may be formed by 400 nucleotides on the engineered polynucleotide side of the dsRNA target and 400 nucleotides on the target RNA side of the dsRNA substrate. A symmetrical internal loop of the present disclosure may be formed by 450 nucleotides on the engineered polynucleotide side of the dsRNA target and 450 nucleotides on the target RNA side of the dsRNA substrate. A symmetrical internal loop of the present disclosure may be formed by 500 nucleotides on the engineered polynucleotide side of the dsRNA target and 500 nucleotides on the target RNA side of the dsRNA substrate. A symmetrical internal loop of the present disclosure may be formed by 600 nucleotides on the engineered polynucleotide side of the dsRNA target and 600 nucleotides on the target RNA side of the dsRNA substrate. A symmetrical internal loop of the present disclosure may be formed by 700 nucleotides on the engineered polynucleotide side of the dsRNA target and 700 nucleotides on the target RNA side of the dsRNA substrate. A symmetrical internal loop of the present disclosure may be formed by 800 nucleotides on the engineered polynucleotide side of the dsRNA target and 800 nucleotides on the target RNA side of the dsRNA substrate. A symmetrical internal loop of the present disclosure may be formed by 900 nucleotides on the engineered polynucleotide side of the dsRNA target and 900 nucleotides on the target RNA side of the dsRNA substrate. A symmetrical internal loop of the present disclosure may be formed by 1000 nucleotides on the engineered polynucleotide side of the dsRNA target and 1000 nucleotides on the target RNA side of the dsRNA substrate.

In an aspect, a double stranded RNA (dsRNA) substrate is formed upon hybridization of an engineered polynucleotide of the present disclosure to a target RNA. An internal loop may be a symmetrical internal loop or an asymmetrical internal loop. An asymmetrical internal loop is formed when a different number of nucleotides is present on each side of the internal loop. For example, an asymmetrical internal loop in a dsRNA substrate of the present disclosure may have different numbers of nucleotides on the engineered polynucleotide side and the target RNA side of the dsRNA substrate. An asymmetrical internal loop of the present disclosure may be formed by from 5 to 150 nucleotides on the engineered polynucleotide side of the dsRNA substrate and from 5 to 150 nucleotides on the target RNA side of the dsRNA substrate, wherein the number of nucleotides is the different on the engineered side of the dsRNA target than the number of nucleotides on the target RNA side of the dsRNA substrate. An asymmetrical internal loop of the present disclosure may be formed by from 5 to 1000 nucleotides on the engineered polynucleotide side of the dsRNA substrate and from 5 to 1000 nucleotides on the target RNA side of the dsRNA substrate, wherein the number of nucleotides is the different on the engineered side of the dsRNA target than the number of nucleotides on the target RNA side of the dsRNA substrate. An asymmetrical internal loop of the present disclosure may be formed by 5 nucleotides on the engineered polynucleotide side of the dsRNA substrate and 6 nucleotides on the target RNA side of the dsRNA substrate. An asymmetrical internal loop of the present disclosure may be formed by 5 nucleotides on the target RNA side of the dsRNA substrate and 6 nucleotides on the engineered polynucleotide side of the dsRNA substrate. An asymmetrical internal loop of the present disclosure may be formed by 5 nucleotides on the engineered polynucleotide side of the dsRNA substrate and 7 nucleotides on the target RNA side of the dsRNA substrate. An asymmetrical internal loop of the present disclosure may be formed by 5 nucleotides on the target RNA side of the dsRNA substrate and 7 nucleotides on the engineered polynucleotide side of the dsRNA substrate. An asymmetrical internal loop of the present disclosure may be formed by 5 nucleotides on the engineered polynucleotide side of the dsRNA substrate and 8 nucleotides internal loop the target RNA side of the dsRNA substrate. An asymmetrical internal loop of the present disclosure may be formed by 5 nucleotides on the target RNA side of the dsRNA substrate and 8 nucleotides on the engineered polynucleotide side of the dsRNA substrate. An asymmetrical internal loop of the present disclosure may be formed by 5 nucleotides on the engineered polynucleotide side of the dsRNA substrate and 9 nucleotides internal loop the target RNA side of the dsRNA substrate. An asymmetrical internal loop of the present disclosure may be formed by 5 nucleotides on the target RNA side of the dsRNA substrate and 9 nucleotides on the engineered polynucleotide side of the dsRNA substrate. An asymmetrical internal loop of the present disclosure may be formed by 5 nucleotides on the engineered polynucleotide side of the dsRNA substrate and 10 nucleotides internal loop the target RNA side of the dsRNA substrate. An asymmetrical internal loop of the present disclosure may be formed by 5 nucleotides on the target RNA side of the dsRNA substrate and 10 nucleotides on the engineered polynucleotide side of the dsRNA substrate. An asymmetrical internal loop of the present disclosure may be formed by 6 nucleotides on the engineered polynucleotide side of the dsRNA substrate and 7 nucleotides internal loop the target RNA side of the dsRNA substrate. An asymmetrical internal loop of the present disclosure may be formed by 6 nucleotides on the target RNA side of the dsRNA substrate and 7 nucleotides on the engineered polynucleotide side of the dsRNA substrate. An asymmetrical internal loop of the present disclosure may be formed by 6 nucleotides on the engineered polynucleotide side of the dsRNA substrate and 8 nucleotides internal loop the target RNA side of the dsRNA substrate. An asymmetrical internal loop of the present disclosure may be formed by 6 nucleotides on the target RNA side of the dsRNA substrate and 8 nucleotides on the engineered polynucleotide side of the dsRNA substrate. An asymmetrical internal loop of the present disclosure may be formed by 6 nucleotides on the engineered polynucleotide side of the dsRNA substrate and 9 nucleotides internal loop the target RNA side of the dsRNA substrate. An asymmetrical internal loop of the present disclosure may be formed by 6 nucleotides on the target RNA side of the dsRNA substrate and 9 nucleotides on the engineered polynucleotide side of the dsRNA substrate. An asymmetrical internal loop of the present disclosure may be formed by 6 nucleotides on the engineered polynucleotide side of the dsRNA substrate and 10 nucleotides internal loop the target RNA side of the dsRNA substrate. An asymmetrical internal loop of the present disclosure may be formed by 6 nucleotides on the target RNA side of the dsRNA substrate and 10 nucleotides on the engineered polynucleotide side of the dsRNA substrate. An asymmetrical internal loop of the present disclosure may be formed by 7 nucleotides on the engineered polynucleotide side of the dsRNA substrate and 8 nucleotides internal loop the target RNA side of the dsRNA substrate. An asymmetrical internal loop of the present disclosure may be formed by 7 nucleotides on the target RNA side of the dsRNA substrate and 8 nucleotides on the engineered polynucleotide side of the dsRNA substrate. An asymmetrical internal loop of the present disclosure may be formed by 7 nucleotides on the engineered polynucleotide side of the dsRNA substrate and 9 nucleotides internal loop the target RNA side of the dsRNA substrate. An asymmetrical internal loop of the present disclosure may be formed by 7 nucleotides on the target RNA side of the dsRNA substrate and 9 nucleotides on the engineered polynucleotide side of the dsRNA substrate. An asymmetrical internal loop of the present disclosure may be formed by 7 nucleotides on the engineered polynucleotide side of the dsRNA substrate and 10 nucleotides internal loop the target RNA side of the dsRNA substrate. An asymmetrical internal loop of the present disclosure may be formed by 7 nucleotides on the target RNA side of the dsRNA substrate and 10 nucleotides on the engineered polynucleotide side of the dsRNA substrate. An asymmetrical internal loop of the present disclosure may be formed by 8 nucleotides on the engineered polynucleotide side of the dsRNA substrate and 9 nucleotides internal loop the target RNA side of the dsRNA substrate. An asymmetrical internal loop of the present disclosure may be formed by 8 nucleotides on the target RNA side of the dsRNA substrate and 9 nucleotides on the engineered polynucleotide side of the dsRNA substrate. An asymmetrical internal loop of the present disclosure may be formed by 8 nucleotides on the engineered polynucleotide side of the dsRNA substrate and 10 nucleotides internal loop the target RNA side of the dsRNA substrate. An asymmetrical internal loop of the present disclosure may be formed by 8 nucleotides on the target RNA side of the dsRNA substrate and 10 nucleotides on the engineered polynucleotide side of the dsRNA substrate. An asymmetrical internal loop of the present disclosure may be formed by 9 nucleotides on the engineered polynucleotide side of the dsRNA substrate and 10 nucleotides internal loop the target RNA side of the dsRNA substrate. An asymmetrical internal loop of the present disclosure may be formed by 9 nucleotides on the target RNA side of the dsRNA substrate and 10 nucleotides on the engineered polynucleotide side of the dsRNA substrate. An asymmetrical internal loop of the present disclosure may be formed by 5 nucleotides on the target RNA side of the dsRNA substrate and 50 nucleotides on the engineered polynucleotide side of the dsRNA substrate. An asymmetrical internal loop of the present disclosure may be formed by 5 nucleotides on the target RNA side of the dsRNA substrate and 100 nucleotides on the engineered polynucleotide side of the dsRNA substrate. An asymmetrical internal loop of the present disclosure may be formed by 5 nucleotides on the target RNA side of the dsRNA substrate and 150 nucleotides on the engineered polynucleotide side of the dsRNA substrate. An asymmetrical internal loop of the present disclosure may be formed by 5 nucleotides on the target RNA side of the dsRNA substrate and 200 nucleotides on the engineered polynucleotide side of the dsRNA substrate. An asymmetrical internal loop of the present disclosure may be formed by 5 nucleotides on the target RNA side of the dsRNA substrate and 300 nucleotides on the engineered polynucleotide side of the dsRNA substrate. An asymmetrical internal loop of the present disclosure may be formed by 5 nucleotides on the target RNA side of the dsRNA substrate and 400 nucleotides on the engineered polynucleotide side of the dsRNA substrate. An asymmetrical internal loop of the present disclosure may be formed by 5 nucleotides on the target RNA side of the dsRNA substrate and 500 nucleotides on the engineered polynucleotide side of the dsRNA substrate. An asymmetrical internal loop of the present disclosure may be formed by 5 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 5 nucleotides on the engineered polynucleotide side of the dsRNA substrate. An asymmetrical internal loop of the present disclosure may be formed by 500 nucleotides on the target RNA side of the dsRNA substrate and 5 nucleotides on the engineered polynucleotide side of the dsRNA substrate. An asymmetrical internal loop of the present disclosure may be formed by 400 nucleotides on the target RNA side of the dsRNA substrate and 5 nucleotides on the engineered polynucleotide side of the dsRNA substrate. An asymmetrical internal loop of the present disclosure may be formed by 300 nucleotides on the target RNA side of the dsRNA substrate and 5 nucleotides on the engineered polynucleotide side of the dsRNA substrate. An asymmetrical internal loop of the present disclosure may be formed by 200 nucleotides on the target RNA side of the dsRNA substrate and 5 nucleotides on the engineered polynucleotide side of the dsRNA substrate. An asymmetrical internal loop of the present disclosure may be formed by 150 nucleotides on the target RNA side of the dsRNA substrate and 5 nucleotides on the engineered polynucleotide side of the dsRNA substrate. An asymmetrical internal loop of the present disclosure may be formed by 100 nucleotides on the target RNA side of the dsRNA substrate and 5 nucleotides on the engineered polynucleotide side of the dsRNA substrate. An asymmetrical internal loop of the present disclosure may be formed by 50 nucleotides on the target RNA side of the dsRNA substrate and 5 nucleotides on the engineered polynucleotide side of the dsRNA substrate. An asymmetrical internal loop of the present disclosure may be formed by 50 nucleotides on the target RNA side of the dsRNA substrate and 100 nucleotides on the engineered polynucleotide side of the dsRNA substrate. An asymmetrical internal loop of the present disclosure may be formed by 50 nucleotides on the target RNA side of the dsRNA substrate and 150 nucleotides on the engineered polynucleotide side of the dsRNA substrate. An asymmetrical internal loop of the present disclosure may be formed by 50 nucleotides on the target RNA side of the dsRNA substrate and 200 nucleotides on the engineered polynucleotide side of the dsRNA substrate. An asymmetrical internal loop of the present disclosure may be formed by 50 nucleotides on the target RNA side of the dsRNA substrate and 300 nucleotides on the engineered polynucleotide side of the dsRNA substrate. An asymmetrical internal loop of the present disclosure may be formed by 50 nucleotides on the target RNA side of the dsRNA substrate and 400 nucleotides on the engineered polynucleotide side of the dsRNA substrate. An asymmetrical internal loop of the present disclosure may be formed by 50 nucleotides on the target RNA side of the dsRNA substrate and 500 nucleotides on the engineered polynucleotide side of the dsRNA substrate. An asymmetrical internal loop of the present disclosure may be formed by 50 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 50 nucleotides on the engineered polynucleotide side of the dsRNA substrate. An asymmetrical internal loop of the present disclosure may be formed by 500 nucleotides on the target RNA side of the dsRNA substrate and 50 nucleotides on the engineered polynucleotide side of the dsRNA substrate. An asymmetrical internal loop of the present disclosure may be formed by 400 nucleotides on the target RNA side of the dsRNA substrate and 50 nucleotides on the engineered polynucleotide side of the dsRNA substrate. An asymmetrical internal loop of the present disclosure may be formed by 300 nucleotides on the target RNA side of the dsRNA substrate and 50 nucleotides on the engineered polynucleotide side of the dsRNA substrate. An asymmetrical internal loop of the present disclosure may be formed by 200 nucleotides on the target RNA side of the dsRNA substrate and 50 nucleotides on the engineered polynucleotide side of the dsRNA substrate. An asymmetrical internal loop of the present disclosure may be formed by 150 nucleotides on the target RNA side of the dsRNA substrate and 50 nucleotides on the engineered polynucleotide side of the dsRNA substrate. An asymmetrical internal loop of the present disclosure may be formed by 100 nucleotides on the target RNA side of the dsRNA substrate and 50 nucleotides on the engineered polynucleotide side of the dsRNA substrate. An asymmetrical internal loop of the present disclosure may be formed by 100 nucleotides on the target RNA side of the dsRNA substrate and 150 nucleotides on the engineered polynucleotide side of the dsRNA substrate. An asymmetrical internal loop of the present disclosure may be formed by 100 nucleotides on the target RNA side of the dsRNA substrate and 200 nucleotides on the engineered polynucleotide side of the dsRNA substrate. An asymmetrical internal loop of the present disclosure may be formed by 100 nucleotides on the target RNA side of the dsRNA substrate and 300 nucleotides on the engineered polynucleotide side of the dsRNA substrate. An asymmetrical internal loop of the present disclosure may be formed by 100 nucleotides on the target RNA side of the dsRNA substrate and 400 nucleotides on the engineered polynucleotide side of the dsRNA substrate. An asymmetrical internal loop of the present disclosure may be formed by 100 nucleotides on the target RNA side of the dsRNA substrate and 500 nucleotides on the engineered polynucleotide side of the dsRNA substrate. An asymmetrical internal loop of the present disclosure may be formed by 100 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 100 nucleotides on the engineered polynucleotide side of the dsRNA substrate. An asymmetrical internal loop of the present disclosure may be formed by 500 nucleotides on the target RNA side of the dsRNA substrate and 100 nucleotides on the engineered polynucleotide side of the dsRNA substrate. An asymmetrical internal loop of the present disclosure may be formed by 400 nucleotides on the target RNA side of the dsRNA substrate and 100 nucleotides on the engineered polynucleotide side of the dsRNA substrate. An asymmetrical internal loop of the present disclosure may be formed by 300 nucleotides on the target RNA side of the dsRNA substrate and 100 nucleotides on the engineered polynucleotide side of the dsRNA substrate. An asymmetrical internal loop of the present disclosure may be formed by 200 nucleotides on the target RNA side of the dsRNA substrate and 100 nucleotides on the engineered polynucleotide side of the dsRNA substrate. An asymmetrical internal loop of the present disclosure may be formed by 150 nucleotides on the target RNA side of the dsRNA substrate and 100 nucleotides on the engineered polynucleotide side of the dsRNA substrate. An asymmetrical internal loop of the present disclosure may be formed by 150 nucleotides on the target RNA side of the dsRNA substrate and 200 nucleotides on the engineered polynucleotide side of the dsRNA substrate. An asymmetrical internal loop of the present disclosure may be formed by 150 nucleotides on the target RNA side of the dsRNA substrate and 300 nucleotides on the engineered polynucleotide side of the dsRNA substrate. An asymmetrical internal loop of the present disclosure may be formed by 150 nucleotides on the target RNA side of the dsRNA substrate and 400 nucleotides on the engineered polynucleotide side of the dsRNA substrate. An asymmetrical internal loop of the present disclosure may be formed by 150 nucleotides on the target RNA side of the dsRNA substrate and 500 nucleotides on the engineered polynucleotide side of the dsRNA substrate. An asymmetrical internal loop of the present disclosure may be formed by 150 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 150 nucleotides on the engineered polynucleotide side of the dsRNA substrate. An asymmetrical internal loop of the present disclosure may be formed by 500 nucleotides on the target RNA side of the dsRNA substrate and 5 nucleotides on the engineered polynucleotide side of the dsRNA substrate. An asymmetrical internal loop of the present disclosure may be formed by 400 nucleotides on the target RNA side of the dsRNA substrate and 150 nucleotides on the engineered polynucleotide side of the dsRNA substrate. An asymmetrical internal loop of the present disclosure may be formed by 300 nucleotides on the target RNA side of the dsRNA substrate and 150 nucleotides on the engineered polynucleotide side of the dsRNA substrate. An asymmetrical internal loop of the present disclosure may be formed by 200 nucleotides on the target RNA side of the dsRNA substrate and 300 nucleotides on the engineered polynucleotide side of the dsRNA substrate. An asymmetrical internal loop of the present disclosure may be formed by 200 nucleotides on the target RNA side of the dsRNA substrate and 400 nucleotides on the engineered polynucleotide side of the dsRNA substrate. An asymmetrical internal loop of the present disclosure may be formed by 200 nucleotides on the target RNA side of the dsRNA substrate and 500 nucleotides on the engineered polynucleotide side of the dsRNA substrate. An asymmetrical internal loop of the present disclosure may be formed by 200 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 200 nucleotides on the engineered polynucleotide side of the dsRNA substrate. An asymmetrical internal loop of the present disclosure may be formed by 500 nucleotides on the target RNA side of the dsRNA substrate and 200 nucleotides on the engineered polynucleotide side of the dsRNA substrate. An asymmetrical internal loop of the present disclosure may be formed by 400 nucleotides on the target RNA side of the dsRNA substrate and 200 nucleotides on the engineered polynucleotide side of the dsRNA substrate. An asymmetrical internal loop of the present disclosure may be formed by 300 nucleotides on the target RNA side of the dsRNA substrate and 200 nucleotides on the engineered polynucleotide side of the dsRNA substrate. An asymmetrical internal loop of the present disclosure may be formed by 300 nucleotides on the target RNA side of the dsRNA substrate and 400 nucleotides on the engineered polynucleotide side of the dsRNA substrate. An asymmetrical internal loop of the present disclosure may be formed by 300 nucleotides on the target RNA side of the dsRNA substrate and 500 nucleotides on the engineered polynucleotide side of the dsRNA substrate. An asymmetrical internal loop of the present disclosure may be formed by 300 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 300 nucleotides on the engineered polynucleotide side of the dsRNA substrate. An asymmetrical internal loop of the present disclosure may be formed by 500 nucleotides on the target RNA side of the dsRNA substrate and 300 nucleotides on the engineered polynucleotide side of the dsRNA substrate. An asymmetrical internal loop of the present disclosure may be formed by 400 nucleotides on the target RNA side of the dsRNA substrate and 300 nucleotides on the engineered polynucleotide side of the dsRNA substrate. An asymmetrical internal loop of the present disclosure may be formed by 400 nucleotides on the target RNA side of the dsRNA substrate and 500 nucleotides on the engineered polynucleotide side of the dsRNA substrate. An asymmetrical internal loop of the present disclosure may be formed by 400 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 400 nucleotides on the engineered polynucleotide side of the dsRNA substrate. An asymmetrical internal loop of the present disclosure may be formed by 500 nucleotides on the target RNA side of the dsRNA substrate and 400 nucleotides on the engineered polynucleotide side of the dsRNA substrate. An asymmetrical internal loop of the present disclosure may be formed by 500 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 500 nucleotides on the engineered polynucleotide side of the dsRNA substrate. In some embodiments, an asymmetrical loop increases efficiency of editing a target A. In some embodiments, an asymmetrical loop that increases efficiency of editing a target A is an asymmetrical bulge that is formed to reduce the number of adenosines in the sequence of the engineered polynucleotide. Non-limiting examples of an asymmetrical loop that increases efficiency of editing a target A are an asymmetrical loop formed by 5 nucleotides on the engineered polynucleotide side of the dsRNA substrate and 20 nucleotide on the target RNA side of the dsRNA substrate; an asymmetrical bulge of formed by 10 nucleotides on the engineered polynucleotide side of the dsRNA substrate and 50 nucleotides on the target RNA side of the dsRNA substrate; an asymmetrical bulge of formed by 60 nucleotides on the engineered polynucleotide side of the dsRNA substrate and 80 nucleotides on the target RNA side of the dsRNA substrate; an asymmetrical bulge of formed by 18 nucleotides on the engineered polynucleotide side of the dsRNA substrate and 24 nucleotides on the target RNA side of the dsRNA substrate; an asymmetrical bulge of formed by 100 nucleotide on the engineered polynucleotide side of the dsRNA substrate and 150 nucleotides on the target RNA side of the dsRNA substrate; an asymmetrical bulge of formed by 70 nucleotide on the engineered polynucleotide side of the dsRNA substrate and 75 nucleotides on the target RNA side of the dsRNA substrate; an asymmetrical bulge of formed by 8 nucleotide on the engineered polynucleotide side of the dsRNA substrate and 15 nucleotides on the target RNA side of the dsRNA substrate; an asymmetrical bulge of formed by 45 nucleotides on the engineered polynucleotide side of the dsRNA substrate and 46 nucleotides on the target RNA side of the dsRNA substrate; an asymmetrical bulge of formed by 45 nucleotides on the engineered polynucleotide side of the dsRNA substrate and 50 nucleotides on the target RNA side of the dsRNA substrate; and an asymmetrical bulge of formed by 7 nucleotides on the engineered polynucleotide side of the dsRNA substrate and 15 nucleotides on the target RNA side of the dsRNA substrate.

Structural features that comprise a bulge or loop can be of any size. In some cases, a bulge or loop comprise at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 600, 700, 800, 900, or 1000 bases. In some cases, a bulge or loop comprise at least about 1-10, 5-15, 10-20, 15-25, 20-30, 1-30, 1-40, 1-50, 1-60, 1-70, 1-80, 1-90, 1-100, 1-110, 1-120, 1-130, 1-140, 1-150, 1-200, 1-250, 1-300, 1-350, 1-400, 1-450, 1-500, 1-600, 1-700, 1-800, 1-900, 1-1000, 20-50, 20-60, 20-70, 20-80, 20-90, 20-100, 20-110, 20-120, 20-130, 20-140, 20-150, 1-200, 1-250, 1-300, 1-350, 1-400, 1-450, 1-500, 1-600, 1-700, 1-800, 1-900, 1-1000, 30-40, 30-50, 30-60, 30-70, 30-80, 30-90, 30-100, 30-110, 30-120, 30-130, 30-140, 30-150, 30-200, 30-250, 30-300, 30-350, 30-400, 30-450, 30-500, 30-600, 30-700, 30-800, 30-900, 30-1000, 40-50, 40-60, 40-70, 40-80, 40-90, 40-100, 40-110, 40-120, 40-130, 40-140, 40-150, 40-200, 40-250, 40-300, 40-350, 40-400, 40-450, 40-500, 40-600, 40-700, 40-800, 40-900, 40-1000, 50-60, 50-70, 50-80, 50-90, 50-100, 50-110, 50-120, 50-130, 50-140, 50-150, 50-200, 50-250, 50-300, 50-350, 50-400, 50-450, 50-500, 50-600, 50-700, 50-800, 50-900, 50-1000, 60-70, 60-80, 60-90, 60-100, 60-110, 60-120, 60-130, 60-140, 60-150, 60-200, 60-250, 60-300, 60-350, 60-400, 60-450, 60-500, 60-600, 60-700, 60-800, 60-900, 60-1000, 70-80, 70-90, 70-100, 70-110, 70-120, 70-130, 70-140, 70-150, 70-200, 70-250, 70-300, 70-350, 70-400, 70-450, 70-500, 70-600, 70-700, 70-800, 70-900, 70-1000, 80-90, 80-100, 80-110, 80-120, 80-130, 80-140, 80-150, 80-200, 80-250, 80-300, 80-350, 80-400, 80-450, 80-500, 80-600, 80-700, 80-800, 80-900, 80-1000, 90-100, 90-110, 90-120, 90-130, 90-140, 90-150, 90-200, 90-250, 90-300, 90-350, 90-400, 90-450, 90-500, 90-600, 90-700, 90-800, 90-900, 90-1000, 100-110, 100-120, 100-130, 100-140, 100-150, 100-200, 100-250, 100-300, 100-350, 100-400, 100-450, 100-500, 100-600, 100-700, 100-800, 100-900, 100-1000, 110-120, 110-130, 110-140, 110-150, 110-200, 110-250, 110-300, 110-350, 110-400, 110-450, 110-500, 110-600, 110-700, 110-800, 110-900, 110-1000, 120-130, 120-140, 120-150, 120-200, 120-250, 120-300, 120-350, 120-400, 120-450, 120-500, 120-600, 120-700, 120-800, 120-900, 120-1000, 130-140, 130-150, 130-200, 130-250, 130-300, 130-350, 130-400, 130-450, 130-500, 130-600, 130-700, 130-800, 130-900, 130-1000, 140-150, 140-200, 140-250, 140-300, 140-350, 140-400, 140-450, 140-500, 140-600, 140-700, 140-800, 140-900, 140-1000, 150-200, 150-250, 150-300, 150-350, 150-400, 150-450, 150-500, 150-600, 150-700, 150-800, 150-900, 150-1000, 200-250, 200-300, 200-350, 200-400, 200-450, 200-500, 200-600, 200-700, 200-800, 200-900, 200-1000, 250-300, 250-350, 250-400, 250-450, 250-500, 250-600, 250-700, 250-800, 250-900, 250-1000, 300-350, 300-400, 300-450, 300-500, 300-600, 300-700, 300-800, 300-900, 300-1000, 350-400, 350-450, 350-500, 350-600, 350-700, 350-800, 350-900, 350-1000, 400-450, 400-500, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, or 900-1000 bases in total.

In some cases, a structural feature can be a structured motif. As disclosed herein, a structured motif comprises two or more structural features in a dsRNA substrate. A structured motif can comprise of any combination of structural features, such as in the above claims, to generate an ideal substrate for ADAR editing at a precise location(s). These structural motifs could be artificially engineered to maximized ADAR editing, and/or these structural motifs can be modeled to recapitulate known ADAR substrates.

In some cases, the engineered polynucleotide comprises an at least partial circularization of a polynucleotide. In some cases, an engineered polynucleotide provided herein can be circularized or in a circular configuration. In some aspects, an at least partially circular polynucleotide lacks a 5′ hydroxyl or a 3′ hydroxyl.

In some embodiments, an engineered polynucleotide can comprise a backbone comprising a plurality of sugar and phosphate moieties covalently linked together. In some cases, a backbone of an engineered polynucleotide can comprise a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an engineered polynucleotide can lack a 5′ reducing hydroxyl, a 3′ reducing hydroxyl, or both, capable of being exposed to a solvent. In some embodiments, a backbone of an engineered polynucleotide can lack a 5′ reducing hydroxyl, a 3′ reducing hydroxyl, or both, capable of being exposed to nucleases. In some embodiments, a backbone of an engineered polynucleotide can lack a 5′ reducing hydroxyl, a 3′ reducing hydroxyl, or both, capable of being exposed to hydrolytic enzymes. In some instances, a backbone of an engineered polynucleotide can be represented as a polynucleotide sequence in a circular 2-dimensional format with one nucleotide after the other. In some instances, a backbone of an engineered polynucleotide can be represented as a polynucleotide sequence in a looped 2-dimensional format with one nucleotide after the other. In some cases, a 5′ hydroxyl, a 3′ hydroxyl, or both, are joined through a phosphorus-oxygen bond. In some cases, a 5′ hydroxyl, a 3′ hydroxyl, or both, are modified into a phosphoester with a phosphorus-containing moiety.

Subject polynucleotides can comprise modifications. A modification can be a substitution, insertion, deletion, chemical modification, physical modification, stabilization, purification, or any combination thereof. In some cases, a modification is a chemical modification. Suitable chemical modifications comprise any one of: 5′adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3phosphorothioate or any combinations thereof.

A modification can be made at any location of a polynucleotide. In some cases, a modification is located in a 5′ or 3′ end. In some cases, a polynucleotide comprises a modification at a base selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150. More than one modification can be made to a polynucleotide. In some cases, a modification can be permanent. In other cases, a modification can be transient. In some cases, multiple modifications are made to a polynucleic acid. A polynucleic acid modification may alter physio-chemical properties of a nucleotide, such as their conformation, polarity, hydrophobicity, chemical reactivity, base-pairing interactions, or any combination thereof.

A modification can also be a phosphorothioate substitute. In some cases, a natural phosphodiester bond may be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases is of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

A polynucleotide can have any frequency of bases. For example, a polynucleotide can have a percent adenine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent cytosine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent thymine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent guanine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent uracil of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%.

In some cases, a polynucleotide can undergo quality control after a modification. In some cases, quality control may include PAGE, HPLC, MS, or any combination thereof. In some cases, a mass of a polynucleotide can be determined. A mass can be determined by LC-MS assay. A mass can be 30,000 amu, 50,000 amu, 70,000 amu, 90,000 amu, 100,000 amu, 120,000 amu, 150,000 amu, 175,000 amu, 200,000 amu, 250,000 amu, 300,000 amu, 350,000 amu, 400,000 amu, to about 500,000 amu. A mass can be of a sodium salt of a polynucleotide.

In some cases, an endotoxin level of a polynucleotide can be determined. A clinically/therapeutically acceptable level of an endotoxin can be less than 3 EU/mL. A clinically/therapeutically acceptable level of an endotoxin can be less than 10 EU/mL. A clinically/therapeutically acceptable level of an endotoxin can be less than 8 EU/mL. A clinically/therapeutically acceptable level of an endotoxin can be less than 5 EU/mL. A clinically/therapeutically acceptable level of an endotoxin can be less than 4 EU/mL. A clinically/therapeutically acceptable level of an endotoxin can be less than 3 EU/mL. A clinically/therapeutically acceptable level of an endotoxin can be less than 2 EU/mL. A clinically/therapeutically acceptable level of an endotoxin can be less than 1 EU/mL. A clinically/therapeutically acceptable level of an endotoxin can be less than 0.5 EU/mL.

In some cases, a polynucleotide can undergo sterility testing. A clinically/therapeutically acceptable level of a sterility testing can be 0 or denoted by no growth on a culture. A clinically/therapeutically acceptable level of a sterility testing can be less than 0.5% growth. A clinically/therapeutically acceptable level of a sterility testing can be less than 1% growth.

In some cases, any one of the polynucleotides that comprise recruiting sequences may also comprise structural features described herein.

Also provided are linear engineered polynucleotides. Linear polynucleotides can substantially lack structural features provided herein. For example, a linear polynucleotide can lack a structural feature or can have less than about 2 structural features or partial structures. A partial structure can comprise a portion of the bases required to achieve a structural feature as described herein.

In other cases, a linear engineered polynucleotide can comprise any one of: 5′ hydroxyl, a 3′ hydroxyl, or both. Any one of these can be capable of being exposed to solvent and maintain linearization.

Compositions and methods provided herein can be utilized to modulate expression of a target. Modulation can refer to altering the expression of a gene or portion thereof at one of various stages, with a view to alleviate a disease or condition associated with the gene or a mutation in the gene. Modulation can be mediated at the level of transcription or post-transcriptionally. Modulating transcription can correct aberrant expression of splice variants generated by a mutation in a gene. In some cases, compositions and methods provided herein can be utilized to regulate translation of a target. Modulation can refer to decreasing or knocking down the expression of a gene or portion thereof by decreasing the abundance of a transcript. The decreasing the abundance of a transcript can be mediated by decreasing the processing, splicing, turnover or stability of the transcript; or by decreasing the accessibility of the transcript to translational machinery such as ribosome. In some cases, an engineered polynucleotide described herein can facilitate a knockdown. A knockdown can be the reduction of the expression of a target RNA. In some cases, a knockdown can be achieved by editing of an mRNA. In some instances, a knockdown can be achieved by targeting an untranslated region of the target RNA, such as a 3′ UTR, a 5′ UTR or both. In some cases, a knockdown can be achieved by targeting a coding region of the target RNA. In some instances, a knockdown can be mediated by an RNA editing enzyme (e.g. ADAR). In some instances, an RNA editing enzyme can cause a knockdown by hydrolytic deamination of multiple adenosines in an RNA. Hydrolytic deamination of multiple adenosines in an RNA can be referred to as hyper-editing. In some cases, hyper-editing can occur in cis (e.g. in an Alu element) or in trans (e.g. in a target RNA by an engineered polynucleotide). In some instances, an RNA editing enzyme can cause a knockdown by editing a target RNA to comprise a premature stop codon or prevent initiation of translation of the target RNA due to an edit in the target RNA.

In some embodiments, the engineered polynucleotide comprises at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182. In some embodiments, the engineered polynucleotide comprising at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182 is used to facilitate editing of a LRRK2 mRNA. In some embodiments, the engineered polynucleotide comprising at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182 is used to facilitate editing of a nucleotide corresponding to the 6055^{th} nucleotide of an LRRK2 mRNA having a sequence of SEQ ID NO: 6. In some embodiments, the engineered polynucleotide comprises at least 60%, 70%, 80%, 85%, 90%, 95%, 97%, 99%, or 100% sequence identity to any one of: SEQ ID NO: 183-SEQ ID NO: 192. In some embodiments, the engineered polynucleotide comprising at least 60%, 70%, 80%, 85%, 90%, 95%, 97%, 99%, or 100% sequence identity to any one of: SEQ ID NO: 183-SEQ ID NO: 192 is used to facilitate editing of an SNCA mRNA. In some embodiments, the engineered polynucleotide comprising at least 60%, 70%, 80%, 85%, 90%, 95%, 97%, 99%, or 100% sequence identity to any one of: SEQ ID NO: 183-SEQ ID NO: 192 is used to facilitate editing of a translation initiation site (TIS) of a SNCA mRNA (e.g., a SNCA mRNA as disclosed herein).

In some embodiments, a composition as disclosed herein comprises an engineered polynucleotide. In some embodiments, the engineered polynucleotide targets a region of a LRRK2 mRNA (e.g., correcting a mutation). In some embodiments, the engineered polynucleotide targets a region of an SNCA mRNA (e.g., resulting in a knockdown of SNCA). In some embodiments, the engineered polynucleotide targets a region of a MAPT mRNA. In some embodiments, the engineered polynucleotide targets a region of a PINK1 mRNA. In some embodiments, the engineered polynucleotide targets a region of a GBA mRNA. In some embodiments, a composition comprises one or more different engineered polynucleotides. For example, a composition comprises an engineered polynucleotide that targets a region of a LRRK2 mRNA and an engineered polynucleotide that targets a region of a SNCA mRNA. In some embodiments, a composition comprises an engineered polynucleotide that targets a region of a GBA mRNA and an engineered polynucleotide that targets a region of a SNCA mRNA. In some embodiments, a composition comprises an engineered polynucleotide that targets a region of a PINK1 mRNA and an engineered polynucleotide that targets a region of a SNCA mRNA. In some embodiments, a composition comprises an engineered polynucleotide that targets a region of a Tau mRNA and an engineered polynucleotide that targets a region of a SNCA mRNA. In some embodiments, a composition comprises an engineered polynucleotide that targets a region of a LRRK2 mRNA, an engineered polynucleotide that targets a region of a Tau, and an engineered polynucleotide that targets a region of a SNCA mRNA.

In some embodiments, the one or more engineered polynucleotides are encoded in the same vector (e.g., a vector disclosed herein). In some embodiments, the one or more engineered polynucleotides encoded in the same vector are the same engineered polynucleotide (e.g., target the same region of a LRRK2 mRNA). In some embodiments, the one or more engineered polynucleotides encoded in the same vector are different engineered polynucleotides (e.g., an engineered polynucleotide that targets a region of a LRRK2 mRNA and an engineered polynucleotide that targets a region of a SNCA mRNA). In some embodiments, two, three, four, or five different engineered polynucleotides are encoded in the same vector. In some embodiments, the one or more engineered polynucleotides are independently encoded in a vector.

### Suitable Targets

Compositions and methods provided herein can be utilized to target suitable RNA polynucleotides and portions thereof. In some cases, a suitable RNA comprises a non-protein coding region, a protein coding region, or both. Exemplary non-protein coding regions include but are not limited to a three prime untranslated region (3′UTR), five prime untranslated region (5′UTR), poly(A) tail, a microRNA response element (MRE), AU-rich element (ARE), or any combination thereof.

In some cases, a suitable RNA to target includes but is not limited to: a precursor-mRNA, a pre-messenger RNA, a messenger RNA, a ribosomal RNA, a transfer RNA, a long non-coding RNA, a small RNA, and any combination thereof.

Exemplary targets can comprise Leucine-rich repeat kinase 2 (LRRK2), Alpha-synuclein (SNCA), glucosylceramidase beta (GBA), PTEN-induced kinase 1 (PINK1), Tau, variants thereof, mutated versions thereof, biologically active fragments of any of these, and combinations thereof.

### Leucine-Rich Repeat Kinase 2 (LRRK2)

Leucine-rich repeat kinase 2 (LRRK2) has been associated with familial and sporadic cases of Parkinson's Disease and immune-related disorders like Crohn's disease. Its aliases include LRRK2, AURA17, DARDARIN, PARK8, RIPK7, ROCO2, or leucine-rich repeat kinase 2. The LRRK2 gene is made up of 51 exons and encodes a 2527 amino-acid protein with a predicted molecular mass of about 286 kDa. The encoded product is a multi-domain protein with kinase and GTPase activities. LRRK2 can be found in various tissues and organs including but not limited to adrenal, appendix, bone marrow, brain, colon, duodenum, endometrium, esophagus, fat, gall bladder, heart, kidney, liver, lung, lymph node, ovary, pancreas, placenta, prostate, salivary gland, skin, small intestine, spleen, stomach, testis, thyroid, and urinary bladder. LRRK2 can be ubiquitously expressed but is generally more abundant in the brain, kidney, and lung tissue. Cellularly, LRRK2 has been found in astrocytes, endothelial cells, microglia, neurons, and peripheral immune cells.

Over 100 amino acid mutations have been identified in LRRK2; six of them—G2019S, R1441C/G/H, Y1699C, and I2020T—have been shown to cause Parkinson's Disease through segregation analysis. G2019S and R1441C are the most common disease-causing mutations in inherited cases. In sporadic cases, these mutations have shown age-dependent penetrance: The percentage of individuals carrying the G2019S mutation that develops the disease jumps from 17% to 85% when the age increases from 50 to 70 years old. In some cases, mutation-carrying individuals never develop the disease.

At its catalytic core, LRRK2 contains the Ras of complex proteins (Roc), C-terminal of ROC (COR), and kinase domains. Multiple protein-protein interaction domains flank this core: an armadillo repeats (ARM) region, an ankyrin repeat (ANK) region, and a leucine-rich repeat (LRR) domain are found in the N-terminus joined by a C-terminal WD40 domain. The G2019S mutation is located within the kinase domain. It has been shown to increase the kinase activity. The R1441C/G/H and Y1699C mutations can decrease the GTPase activity of the Roc domain. Genome-wide association study has found that common variations in LRRK2 increase the risk of developing sporadic Parkinson's Disease. While some of these variations are nonconservative mutations that affect the protein's binding or catalytic activities, others modulate its expression. These results suggest that specific alleles or haplotypes can regulate LRRK2 expression.

Pro-inflammatory signals upregulate LRRK2 expression in various immune cell types, suggesting that LRRK2 is a critical regulator in the immune response. Studies have found that both systemic and central nervous system (CNS) inflammation are involved in Parkinson's Disease's symptoms. Moreover, LRRK2 mutations associated with Parkinson's Disease modulate its expression levels in response to inflammatory stimuli. Many mutations in LRRK2 are associated with immune-related disorders such as inflammatory bowel disease (e.g., Crohn's Disease). For example, both G2019S and N2081D increase LRRK2's kinase activity and are over-represented in Crohn's Disease patients in specific populations. Because of its critical role in these disorders, LRRK2 is an important therapeutic target for Parkinson's Disease and Crohn's Disease. In particular, many mutations, such as point mutations including G2019S, play roles in developing these diseases, making LRRK2 an attractive for therapeutic strategy such as RNA editing.

LRRK2 is encoded by the mRNA sequence of Table 1. In some cases, a region of LRRK2 can be targeted utilizing compositions provided herein. In some cases, at least a portion of an exon or intron of the LRRK2 mRNA can be targeted by an engineered polynucleotide as described herein. In some embodiments, at least a portion of a region of a non-coding sequence of the LRRK2 mRNA, such as the 5′UTR and 3′UTR, can be targeted by an engineered polynucleotide as described herein. In some cases, an editing of a nucleotide base of a 5′UTR can result in regulating translation of a target RNA, such as a polynucleotide encoding a LRRK2 polypeptide. In other cases, a region of the coding sequence of the LRRK2 mRNA can be targeted by an engineered polynucleotide as described herein. In some cases, a region targeted by an engineered polynucleotide described herein comprises a region from a target RNA, wherein the target RNA comprises at least 80%, 85%, 90%, 95%, 97%, or 99% sequence identity to any one of SEQ ID NO: 5 to SEQ ID NO: 14. In some cases, a region targeted by an engineered polynucleotide described herein comprises a region from a target RNA, wherein the target RNA comprises at 100% sequence identity to any one of SEQ ID NO: 5 to SEQ ID NO: 14. Suitable regions of a target RNA include but are not limited to a repeat domain, Ras-of-complex (Roc) GTPase domain, a kinase domain, a WD40 domain, and a C-terminal of Roc (COR) domain, and combinations thereof. In some aspects, a suitable target region of a target RNA can be located in the kinase domain of LRRK2. In some embodiments, a region of a target RNA is any region that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides in length, from any one of SEQ ID NO: 5 to SEQ ID NO: 14. In some embodiments, an engineered polynucleotide as described herein has at least 70%, 75%, 80%, 85%, 90%, 95%, 98%, 99%, or 100% complementarity to a region as described herein from any one of SEQ ID NO: 5 to SEQ ID NO: 14.

In some cases, an exon of the LRRK2 gene is targeted by an engineered polynucleotide as described herein. For example, a suitable target region of a target RNA can comprise exon 41 of LRRK2. A nucleotide codon in exon 41 is implicated in a mutation comprising a glycine to serine substitution (G2019S) located within the protein kinase domain encoded by exon 41.

In an embodiment, a specific nucleotide residue can be targeted utilizing compositions and methods provided herein. Specific nucleotide residues can comprise point mutations as compared to a wildtype sequence such as that provided in Table 1. In some cases, a target nucleotide residue can be position 6190 of the LRRK2 mRNA of SEQ ID NO: 6. Therefore, in some embodiments, an engineered polynucleotide, for example, targets a region comprising the nucleotide residue of position 6190 of SEQ ID NO: 6. In some embodiments, an engineered polynucleotide comprises a targeting sequence that is at least partially complementary to a region of the target RNA, wherein the region of the target RNA comprises at least 60%, 70%, 80%, 85%, 90%, 95%, 97%, 99%, or 100% sequence identity to SEQ ID NO: 73 or SEQ ID NO: 74.

TABLE 1
Human LRRK2 mRNA Isoform Sequences. Sequences obtained from NCBI
LRRK2 gene ID: 120892; Assembly GRCh38.p13
(GCF 000001405.39); NC_000012.12 (40224890 . . . 40369285)

| SEQ ID NO: | Isoform | mRNA Sequence |
|---|---|---|
| 5 | Isoform 1 | GGGGCCCGCGGGGAGCGCTGGCTGCGGGCGGTGAGCTGAGCTCG |
| | | CCCCCGGGGAGCTGTGGCCGGCGCCCCTGCCGGTTCCCTGAGCA |
| | | GCGGACGTTCATGCTGGGAGGGCGGCGGGTTGGAAGCAGGTGCC |
| | | ACCATGGCTAGTGGCAGCTGTCAGGGGTGCGAAGAGGACGAGGA |
| | | AACTCTGAAGAAGTTGATAGTCAGGCTGAACAATGTCCAGGAAG |
| | | GAAAACAGATAGAAACGCTGGTCCAAATCCTGGAGGATCTGCTG |
| | | GTGTTCACGTACTCCGAGCGCGCCTCCAAGTTATTTCAAGGCAAA |
| | | AATATCCATGTGCCTCTGTTGATCGTCTTGGACTCCTATATGAGA |
| | | GTCGCGAGTGTGCAGCAGGTGGGTTGGTCACTTCTGTGCAAATTA |
| | | ATAGAAGTCTGTCCAGGTACAATGCAAAGCTTAATGGGACCCCA |
| | | GGATGTTGGAAATGATTGGGAAGTCCTTGGTGTTCACCAATTGAT |
| | | TCTTAAAATGCTAACAGTTCATAATGCCAGTGTAAACTTGTCAGT |
| | | GATTGGACTGAAGACCTTAGATCTCCTCCTAACTTCAGGTAAAAT |
| | | CACCTTGCTGATATTGGATGAAGAAAGTGATATTTTCATGTTAAT |
| | | TTTTGATGCCATGCACTCATTTCCAGCCAATGATGAAGTCCAGAA |
| | | ACTTGGATGCAAAGCTTTACATGTGCTGTTTGAGAGAGTCTCAGA |
| | | GGAGCAACTGACTGAATTTGTTGAGAACAAAGATTATATGATATT |
| | | GTTAAGTGCGTTAACAAATTTTAAAGATGAAGAGGAAATTGTGC |
| | | TTCATGTGCTGCATTGTTTACATTCCCTAGCGATTCCTTGCAATAA |
| | | TGTGGAAGTCCTCATGAGTGGCAATGTCAGGTGTTATAATATTGT |
| | | GGTGGAAGCTATGAAAGCATTCCCTATGAGTGAAAGAATTCAAG |
| | | AAGTGAGTTGCTGTTTGCTCCATAGGCTTACATTAGGTAATTTTTT |
| | | CAATATCCTGGTATTAAACGAAGTCCATGAGTTTGTGGTGAAAGC |
| | | TGTGCAGCAGTACCCAGAGAATGCAGCATTGCAGATCTCAGCGC |
| | | TCAGCTGTTTGGCCCTCCTCACTGAGACTATTTTCTTAAATCAAG |
| | | ATTTAGAGGAAAAGAATGAGAATCAAGAGAATGATGATGAGGG |
| | | GGAAGAAGATAAATTGTTTTGGCTGGAAGCCTGTTACAAAGCAT |
| | | TAACGTGGCATAGAAAGAACAAGCACGTGCAGGAGGCCGCATGC |
| | | TGGGCACTAAATAATCTCCTTATGTACCAAAACAGTTTACATGAG |
| | | AAGATTGGAGATGAAGATGGCCATTTCCCAGCTCATAGGGAAGT |
| | | GATGCTCTCCATGCTGATGCATTCTTCATCAAAGGAAGTTTTCCA |
| | | GGCATCTGCGAATGCATTGTCAACTCTCTTAGAACAAAATGTTAA |
| | | TTTCAGAAAAATACTGTTATCAAAAGGAATACACCTGAATGTTTT |
| | | GGAGTTAATGCAGAAGCATATACATTCTCCTGAAGTGGCTGAAA |
| | | GTGGCTGTAAAATGCTAAATCATCTTTTTGAAGGAAGCAACACTT |
| | | CCCTGGATATAATGGCAGCAGTGGTCCCCAAAATACTAACAGTT |
| | | ATGAAACGTCATGAGACATCATTACCAGTGCAGCTGGAGGCGCT |
| | | TCGAGCTATTTTACATTTTATAGTGCCTGGCATGCCAGAAGAATC |
| | | CAGGGAGGATACAGAATTTCATCATAAGCTAAATATGGTTAAAA |
| | | AACAGTGTTTCAAGAATGATATTCACAAACTGGTCCTAGCAGCTT |
| | | TGAACAGGTTCATTGGAAATCCTGGGATTCAGAAATGTGGATTA |
| | | AAAGTAATTTCTTCTATTGTACATTTTCCTGATGCATTAGAGATGT |
| | | TATCCCTGGAAGGTGCTATGGATTCAGTGCTTCACACACTGCAGA |
| | | TGTATCCAGATGACCAAGAAATTCAGTGTCTGGGTTTAAGTCTTA |
| | | TAGGATACTTGATTACAAAGAAGAATGTGTTCATAGGAACTGGA |
| | | CATCTGCTGGCAAAAATTCTGGTTTCCAGCTTATACCGATTTAAG |
| | | GATGTTGCTGAAATACAGACTAAAGGATTTCAGACAATCTTAGC |
| | | AATCCTCAAATTGTCAGCATCTTTTTCTAAGCTGCTGGTGCATCAT |
| | | TCATTTGACTTAGTAATATTCCATCAAATGTCTTCCAATATCATGG |
| | | AACAAAAGGATCAACAGTTTCTAAACCTCTGTTGCAAGTGTTTTG |
| | | CAAAAGTAGCTATGGATGATTACTTAAAAAATGTGATGCTAGAG |
| | | AGAGCGTGTGATCAGAATAACAGCATCATGGTTGAATGCTTGCTT |
| | | CTATTGGGAGCAGATGCCAATCAAGCAAAGGAGGGATCTTCTTT |
| | | AATTTGTCAGGTATGTGAGAAAGAGAGCAGTCCCAAATTGGTGG |
| | | AACTCTTACTGAATAGTGGATCTCGTGAACAAGATGTACGAAAA |
| | | GCGTTGACGATAAGCATTGGGAAAGGTGACAGCCAGATCATCAG |
| | | CTTGCTCTTAAGGAGGCTGGCCCTGGATGTGGCCAACAATAGCAT |
| | | TTGCCTTGGAGGATTTTGTATAGGAAAAGTTGAACCTTCTTGGCT |
| | | TGGTCCTTTATTTCCAGATAAGACTTCTAATTTAAGGAAACAAAC |
| | | AAATATAGCATCTACACTAGCAAGAATGGTGATCAGATATCAGA |
| | | TGAAAAGTGCTGTGGAAGAAGGAACAGCCTCAGGCAGCGATGGA |
| | | AATTTTTCTGAAGATGTGCTGTCTAAATTTGATGAATGGACCTTT |
| | | ATTCCTGACTCTTCTATGGACAGTGTGTTTGCTCAAAGTGATGAC |
| | | CTGGATAGTGAAGGAAGTGAAGGCTCATTTCTTGTGAAAAAGAA |
| | | ATCTAATTCAATTAGTGTAGGAGAATTTTACCGAGATGCCGTATT |
| | | ACAGCGTTGCTCACCAAATTTGCAAAGACATTCCAATTCCTTGGG |
| | | GCCCATTTTTGATCATGAAGATTTACTGAAGCGAAAAAGAAAAA |
| | | TATTATCTTCAGATGATTCACTCAGGTCATCAAAACTTCAATCCC |
| | | ATATGAGGCATTCAGACAGCATTTCTTCTCTGGCTTCTGAGAGAG |
| | | AATATATTACATCACTAGACCTTTCAGCAAATGAACTAAGAGATA |
| | | TTGATGCCCTAAGCCAGAAATGCTGTATAAGTGTTCATTTGGAGC |
| | | ATCTTGAAAAGCTGGAGCTTCACCAGAATGCACTCACGAGCTTTC |
| | | CACAACAGCTATGTGAAACTCTGAAGAGTTTGACACATTTGGACT |
| | | TGCACAGTAATAAATTTACATCATTTCCTTCTTATTTGTTGAAAAT |
| | | GAGTTGTATTGCTAATCTTGATGTCTCTCGAAATGACATTGGACC |
| | | CTCAGTGGTTTTAGATCCTACAGTGAAATGTCCAACTCTGAAACA |
| | | GTTTAACCTGTCATATAACCAGCTGTCTTTTGTACCTGAGAACCT |
| | | CACTGATGTGGTAGAGAAACTGGAGCAGCTCATTTTAGAAGGAA |
| | | ATAAAATATCAGGGATATGCTCCCCCTTGAGACTGAAGGAACTG |
| | | AAGATTTTAAACCTTAGTAAGAACCACATTTCATCCCTATCAGAG |
| | | AACTTTCTTGAGGCTTGTCCTAAAGTGGAGAGTTTCAGTGCCAGA |
| | | ATGAATTTTCTTGCTGCTATGCCTTTCTTGCCTCCTTCTATGACAA |
| | | TCCTAAAATTATCTCAGAACAAATTTTCCTGTATTCCAGAAGCAA |
| | | TTTTAAATCTTCCACACTTGCGGTCTTTAGATATGAGCAGCAATG |
| | | ATATTCAGTACCTACCAGGTCCCGCACACTGGAAATCTTTGAACT |
| | | TAAGGGAACTCTTATTTAGCCATAATCAGATCAGCATCTTGGACT |
| | | TGAGTGAAAAAGCATATTTATGGTCTAGAGTAGAGAAACTGCAT |
| | | CTTTCTCACAATAAACTGAAAGAGATTCCTCCTGAGATTGGCTGT |
| | | CTTGAAAATCTGACATCTCTGGATGTCAGTTACAACTTGGAACTA |
| | | AGATCCTTTCCCAATGAAATGGGGAAATTAAGCAAAATATGGGA |
| | | TCTTCCTTTGGATGAACTGCATCTTAACTTTGATTTTAAACATATA |
| | | GGATGTAAAGCCAAAGACATCATAAGGTTTCTTCAACAGCGATT |
| | | AAAAAAGGCTGTGCCTTATAACCGAATGAAACTTATGATTGTGG |
| | | GAAATACTGGGAGTGGTAAAACCACCTTATTGCAGCAATTAATG |
| | | AAAACCAAGAAATCAGATCTTGGAATGCAAAGTGCCACAGTTGG |
| | | CATAGATGTGAAAGACTGGCCTATCCAAATAAGAGACAAAAGAA |
| | | AGAGAGATCTCGTCCTAAATGTGTGGGATTTTGCAGGTCGTGAGG |
| | | AATTCTATAGTACTCATCCCCATTTTATGACGCAGCGAGCATTGT |
| | | ACCTTGCTGTCTATGACCTCAGCAAGGGACAGGCTGAAGTTGATG |
| | | CCATGAAGCCTTGGCTCTTCAATATAAAGGCTCGCGCTTCTTCTT |
| | | CCCCTGTGATTCTCGTTGGCACACATTTGGATGTTTCTGATGAGA |
| | | AGCAACGCAAAGCCTGCATGAGTAAAATCACCAAGGAACTCCTG |
| | | AATAAGCGAGGGTTCCCTGCCATACGAGATTACCACTTTGTGAAT |
| | | GCCACCGAGGAATCTGATGCTTTGGCAAAACTTCGGAAAACCAT |
| | | CATAAACGAGAGCCTTAATTTCAAGATCCGAGATCAGCTTGTTGT |
| | | TGGACAGCTGATTCCAGACTGCTATGTAGAACTTGAAAAAATCAT |
| | | TTTATCGGAGCGTAAAAATGTGCCAATTGAATTTCCCGTAATTGA |
| | | CCGGAAACGATTATTACAACTAGTGAGAGAAAATCAGCTGCAGT |
| | | TAGATGAAAATGAGCTTCCTCACGCAGTTCACTTTCTAAATGAAT |
| | | CAGGAGTCCTTCTTCATTTTCAAGACCCAGCACTGCAGTTAAGTG |
| | | ACTTGTACTTTGTGGAACCCAAGTGGCTTTGTAAAATCATGGCAC |
| | | AGATTTTGACAGTGAAAGTGGAAGGTTGTCCAAAACACCCTAAG |
| | | GGCATTATTTCGCGTAGAGATGTGGAAAAATTTCTTTCAAAAAAA |
| | | AGGAAATTTCCAAAGAACTACATGTCACAGTATTTTAAGCTCCTA |
| | | GAAAAATTCCAGATTGCTTTGCCAATAGGAGAAGAATATTTGCTG |
| | | GTTCCAAGCAGTTTGTCTGACCACAGGCCTGTGATAGAGCTTCCC |
| | | CATTGTGAGAACTCTGAAATTATCATCCGACTATATGAAATGCCT |
| | | TATTTTCCAATGGGATTTTGGTCAAGATTAATCAATCGATTACTT |
| | | GAGATTTCACCTTACATGCTTTCAGGGAGAGAACGAGCACTTCGC |
| | | CCAAACAGAATGTATTGGCGACAAGGCATTTACTTAAATTGGTCT |
| | | CCTGAAGCTTATTGTCTGGTAGGATCTGAAGTCTTAGACAATCAT |
| | | CCAGAGAGTTTCTTAAAAATTACAGTTCCTTCTTGTAGAAAAGGC |
| | | TGTATTCTTTTGGGCCAAGTTGTGGACCACATTGATTCTCTCATGG |
| | | AAGAATGGTTTCCTGGGTTGCTGGAGATTGATATTTGTGGTGAAG |
| | | GAGAAACTCTGTTGAAGAAATGGGCATTATATAGTTTTAATGATG |
| | | GTGAAGAACATCAAAAAATCTTACTTGATGACTTGATGAAGAAA |
| | | GCAGAGGAAGGAGATCTCTTAGTAAATCCAGATCAACCAAGGCT |
| | | CACCATTCCAATATCTCAGATTGCCCCTGACTTGATTTTGGCTGA |
| | | CCTGCCTAGAAATATTATGTTGAATAATGATGAGTTGGAATTTGA |
| | | ACAAGCTCCAGAGTTTCTCCTAGGTGATGGCAGTTTTGGATCAGT |
| | | TTACCGAGCAGCCTATGAAGGAGAAGAAGTGGCTGTGAAGATTT |
| | | TTAATAAACATACATCACTCAGGCTGTTAAGACAAGAGCTTGTGG |
| | | TGCTTTGCCACCTCCACCACCCCAGTTTGATATCTTTGCTGGCAGC |
| | | TGGGATTCGTCCCCGGATGTTGGTGATGGAGTTAGCCTCCAAGGG |
| | | TTCCTTGGATCGCCTGCTTCAGCAGGACAAAGCCAGCCTCACTAG |
| | | AACCCTACAGCACAGGATTGCACTCCACGTAGCTGATGGTTTGAG |
| | | ATACCTCCACTCAGCCATGATTATATACCGAGACCTGAAACCCCA |
| | | CAATGTGCTGCTTTTCACACTGTATCCCAATGCTGCCATCATTGC |
| | | AAAGATTGCTGACTACGGCATTGCTCAGTACTGCTGTAGAATGGG |
| | | GATAAAAACATCAGAGGGCACACCAGGGTTTCGTGCACCTGAAG |
| | | TTGCCAGAGGAAATGTCATTTATAACCAACAGGCTGATGTTTATT |
| | | CATTTGGTTTACTACTCTATGACATTTTGACAACTGGAGGTAGAA |
| | | TAGTAGAGGGTTTGAAGTTTCCAAATGAGTTTGATGAATTAGAAA |
| | | TACAAGGAAAATTACCTGATCCAGTTAAAGAATATGGTTGTGCCC |
| | | CATGGCCTATGGTTGAGAAATTAATTAAACAGTGTTTGAAAGAA |
| | | AATCCTCAAGAAAGGCCTACTTCTGCCCAGGTCTTTGACATTTTG |
| | | AATTCAGCTGAATTAGTCTGTCTGACGAGACGCATTTTATTACCT |
| | | AAAAACGTAATTGTTGAATGCATGGTTGCTACACATCACAACAG |
| | | CAGGAATGCAAGCATTTGGCTGGGCTGTGGGCACACCGACAGAG |
| | | GACAGCTCTCATTTCTTGACTTAAATACTGAAGGATACACTTCTG |
| | | AGGAAGTTGCTGATAGTAGAATATTGTGCTTAGCCTTGGTGCATC |
| | | TTCCTGTTGAAAAGGAAAGCTGGATTGTGTCTGGGACACAGTCTG |
| | | GTACTCTCCTGGTCATCAATACCGAAGATGGGAAAAAGAGACAT |
| | | ACCCTAGAAAAGATGACTGATTCTGTCACTTGTTTGTATTGCAAT |
| | | TCCTTTTCCAAGCAAAGCAAACAAAAAAATTTTCTTTTGGTTGGA |
| | | ACCGCTGATGGCAAGTTAGCAATTTTTGAAGATAAGACTGTTAAG |
| | | CTTAAAGGAGCTGCTCCTTTGAAGATACTAAATATAGGAAATGTC |
| | | AGTACTCCATTGATGTGTTTGAGTGAATCCACAAATTCAACGGAA |
| | | AGAAATGTAATGTGGGGAGGATGTGGCACAAAGATTTTCTCCTTT |
| | | TCTAATGATTTCACCATTCAGAAACTCATTGAGACAAGAACAAGC |
| | | CAACTGTTTTCTTATGCAGCTTTCAGTGATTCCAACATCATAACA |
| | | GTGGTGGTAGACACTGCTCTCTATATTGCTAAGCAAAATAGCCCT |
| | | GTTGTGGAAGTGTGGGATAAGAAAACTGAAAAACTCTGTGGACT |
| | | AATAGACTGCGTGCACTTTTTAAGGGAGGTAATGGTAAAAGAAA |
| | | ACAAGGAATCAAAACACAAAATGTCTTATTCTGGGAGAGTGAAA |
| | | ACCCTCTGCCTTCAGAAGAACACTGCTCTTTGGATAGGAACTGGA |
| | | GGAGGCCATATTTTACTCCTGGATCTTTCAACTCGTCGACTTATA |
| | | CGTGTAATTTACAACTTTTGTAATTCGGTCAGAGTCATGATGACA |
| | | GCACAGCTAGGAAGCCTTAAAAATGTCATGCTGGTATTGGGCTA |
| | | CAACCGGAAAAATACTGAAGGTACACAAAAGCAGAAAGAGATA |
| | | CAATCTTGCTTGACCGTTTGGGACATCAATCTTCCACATGAAGTG |
| | | CAAAATTTAGAAAAACACATTGAAGTGAGAAAAGAATTAGCTGA |
| | | AAAAATGAGACGAACATCTGTTGAGTAAGAGAGAAATAGGAATT |
| | | GTCTTTGGATAGGAAAATTATTCTCTCCTCTTGTAAATATTTATTT |
| | | TAAAAATGTTCACATGGAAAGGGTACTCACATTTTTTGAAATAGC |
| | | TCGTGTGTATGAAGGAATGTTATTATTTTTAATTTAAATATATGTA |
| | | AAAATACTTACCAGTAAATGTGTATTTTAAAGAACTATTTAAAAC |
| | | ACAATGTTATATTTCTTATAAATACCAGTTACTTTCGTTCATTAAT |
| | | TAATGAAAATAAATCTGTGAAGTACCTAATTTAAGTACTCATACT |
| | | AAAATTTATAAGGCCGATAATTTTTTGTTTTCTTGTCTGTAATGGA |
| | | GGTAAACTTTATTTTAAATTCTGTGCTTAAGACAGGACTATTGCT |
| | | TGTCGATTTTTCTAGAAATCTGCACGGTATAATGAAAATATTAAG |
| | | ACAGTTTCCCATGTAATGTATTCCTTCTTAGATTGCATCGAAATG |
| | | CACTATCATATATGCTTGTAAATATTCAAATGAATTTGCACTAAT |
| | | AAAGTCCTTTGTTGGTATGTGAATTCTCTTTGTTGCTGTTGCAAAC |
| | | AGTGCATCTTACACAACTTCACTCAATTCAAAAGAAAACTCCATT |
| | | AAAAGTACTAATGAAAAAACATGACATACTGTCAAAGTCCTCAT |
| | | ATCTAGGAAAGACACAGAAACTCTCTTTGTCACAGAAACTCTCTG |
| | | TGTCTTTCCTAGACATAATAGAGTTGTTTTTCAACTCTATGTTTGA |
| | | ATGTGGATACCCTGAATTTTGTATAATTAGTGTAAATACAGTGTT |
| | | CAGTCCTTCAAGTGATATTTTTATTTTTTTATTCATACCACTAGCT |
| | | ACTTGTTTTCTAATCTGCTTCATTCTAATGCTTATATTCATCTTTTC |
| | | CCTAAATTTGTGATGCTGCAGATCCTACATCATTCAGATAGAAAC |
| | | CTTTTTTTTTTTCAGAATTATAGAATTCCACAGCTCCTACCAAGAC |
| | | CATGAGGATAAATATCTAACACTTTTCAGTTGCTGAAGGAGAAA |
| | | GGAGCTTTAGTTATGATGGATAAAAATATCTGCCACCCTAGGCTT |
| | | CCAAATTATACTTAAATTGTTTACATAGCTTACCACAATAGGAGT |
| | | ATCAGGGCCAAATACCTATGTAATAATTTGAGGTCATTTCTGCTT |
| | | TAGGAAAAGTACTTTCGGTAAATTCTTTGGCCCTGACCAGTATTC |
| | | ATTATTTCAGATAATTCCCTGTGATAGGACAACTAGTACATTTAA |
| | | TATTCTCAGAACTTATGGCATTTTACTATGTGAAAACTTTAAATTT |
| | | ATTTATATTAAGGGTAATCAAATTCTTAAAGATGAAAGATTTTCT |
| | | GTATTTTAAAGGAAGCTATGCTTTAACTTGTTATGTAATTAACAA |
| | | AAAAATCATATATAATAGAGCTCTTTGTTCCAGTGTTATCTCTTTC |
| | | ATTGTTACTTTGTATTTGCAATTTTTTTTACCAAAGACAAATTAAA |
| | | AAAATGAATACCATATTTAAATGGAATAATAAAGGTTTTTTAAAA |
| | | ACTTTAAA |
| 6 | X1 | CCTGAGTGGGGGAGGAGGAAGCCGAGCAGGAGGGCTCCGGAGA |
| | | GGGAGGGCAACGCGGGGCGGGGAGCTGCCTCCTTCCTCATAAAC |
| | | AGGCGGGCGTGGGCGCCGATGGGGCCCGCGGGGAGCGCTGGCTG |
| | | CGGGCGGTGAGCTGAGCTCGCCCCCGGGGAGCTGTGGCCGGCGC |
| | | CCCTGCCGGTTCCCTGAGCAGCGGACGTTCATGCTGGGAGGGCG |
| | | GCGGGTTGGAAGCAGGTGCCACCATGGCTAGTGGCAGCTGTCAG |
| | | GGGTGCGAAGAGGACGAGGAAACTCTGAAGAAGTTGATAGTCAG |
| | | GCTGAACAATGTCCAGGAAGGAAAACAGATAGAAACGCTGGTCC |
| | | AAATCCTGGAGGATCTGCTGGTGTTCACGTACTCCGAGCGCGCCT |
| | | CCAAGTTATTTCAAGGCAAAAATATCCATGTGCCTCTGTTGATCG |
| | | TCTTGGACTCCTATATGAGAGTCGCGAGTGTGCAGCAGGTGGGTT |
| | | GGTCACTTCTGTGCAAATTAATAGAAGTCTGTCCAGGTACAATGC |
| | | AAAGCTTAATGGGACCCCAGGATGTTGGAAATGATTGGGAAGTC |
| | | CTTGGTGTTCACCAATTGATTCTTAAAATGCTAACAGTTCATAAT |
| | | GCCAGTGTAAACTTGTCAGTGATTGGACTGAAGACCTTAGATCTC |
| | | CTCCTAACTTCAGGTAAAATCACCTTGCTGATATTGGATGAAGAA |
| | | AGTGATATTTTCATGTTAATTTTTGATGCCATGCACTCATTTCCAG |
| | | CCAATGATGAAGTCCAGAAACTTGGATGCAAAGCTTTACATGTG |
| | | CTGTTTGAGAGAGTCTCAGAGGAGCAACTGACTGAATTTGTTGAG |
| | | AACAAAGATTATATGATATTGTTAAGTGCGTTAACAAATTTTAAA |
| | | GATGAAGAGGAAATTGTGCTTCATGTGCTGCATTGTTTACATTCC |
| | | CTAGCGATTCCTTGCAATAATGTGGAAGTCCTCATGAGTGGCAAT |
| | | GTCAGGTGTTATAATATTGTGGTGGAAGCTATGAAAGCATTCCCT |
| | | ATGAGTGAAAGAATTCAAGAAGTGAGTTGCTGTTTGCTCCATAG |
| | | GCTTACATTAGGTAATTTTTTCAATATCCTGGTATTAAACGAAGT |
| | | CCATGAGTTTGTGGTGAAAGCTGTGCAGCAGTACCCAGAGAATG |
| | | CAGCATTGCAGATCTCAGCGCTCAGCTGTTTGGCCCTCCTCACTG |
| | | AGACTATTTTCTTAAATCAAGATTTAGAGGAAAAGAATGAGAAT |
| | | CAAGAGAATGATGATGAGGGGGAAGAAGATAAATTGTTTTGGCT |
| | | GGAAGCCTGTTACAAAGCATTAACGTGGCATAGAAAGAACAAGC |
| | | ACGTGCAGGAGGCCGCATGCTGGGCACTAAATAATCTCCTTATGT |
| | | ACCAAAACAGTTTACATGAGAAGATTGGAGATGAAGATGGCCAT |
| | | TTCCCAGCTCATAGGGAAGTGATGCTCTCCATGCTGATGCATTCT |
| | | TCATCAAAGGAAGTTTTCCAGGCATCTGCGAATGCATTGTCAACT |
| | | CTCTTAGAACAAAATGTTAATTTCAGAAAAATACTGTTATCAAAA |
| | | GGAATACACCTGAATGTTTTGGAGTTAATGCAGAAGCATATACAT |
| | | TCTCCTGAAGTGGCTGAAAGTGGCTGTAAAATGCTAAATCATCTT |
| | | TTTGAAGGAAGCAACACTTCCCTGGATATAATGGCAGCAGTGGT |
| | | CCCCAAAATACTAACAGTTATGAAACGTCATGAGACATCATTACC |
| | | AGTGCAGCTGGAGGCGCTTCGAGCTATTTTACATTTTATAGTGCC |
| | | TGGCATGCCAGAAGAATCCAGGGAGGATACAGAATTTCATCATA |
| | | AGCTAAATATGGTTAAAAAACAGTGTTTCAAGAATGATATTCAC |
| | | AAACTGGTCCTAGCAGCTTTGAACAGGTTCATTGGAAATCCTGGG |
| | | ATTCAGAAATGTGGATTAAAAGTAATTTCTTCTATTGTACATTTTC |
| | | CTGATGCATTAGAGATGTTATCCCTGGAAGGTGCTATGGATTCAG |
| | | TGCTTCACACACTGCAGATGTATCCAGATGACCAAGAAATTCAGT |
| | | GTCTGGGTTTAAGTCTTATAGGATACTTGATTACAAAGAAGAATG |
| | | TGTTCATAGGAACTGGACATCTGCTGGCAAAAATTCTGGTTTCCA |
| | | GCTTATACCGATTTAAGGATGTTGCTGAAATACAGACTAAAGGAT |
| | | TTCAGACAATCTTAGCAATCCTCAAATTGTCAGCATCTTTTTCTAA |
| | | GCTGCTGGTGCATCATTCATTTGACTTAGTAATATTCCATCAAAT |
| | | GTCTTCCAATATCATGGAACAAAAGGATCAACAGTTTCTAAACCT |
| | | CTGTTGCAAGTGTTTTGCAAAAGTAGCTATGGATGATTACTTAAA |
| | | AAATGTGATGCTAGAGAGAGCGTGTGATCAGAATAACAGCATCA |
| | | TGGTTGAATGCTTGCTTCTATTGGGAGCAGATGCCAATCAAGCAA |
| | | AGGAGGGATCTTCTTTAATTTGTCAGGTATGTGAGAAAGAGAGC |
| | | AGTCCCAAATTGGTGGAACTCTTACTGAATAGTGGATCTCGTGAA |
| | | CAAGATGTACGAAAAGCGTTGACGATAAGCATTGGGAAAGGTGA |
| | | CAGCCAGATCATCAGCTTGCTCTTAAGGAGGCTGGCCCTGGATGT |
| | | GGCCAACAATAGCATTTGCCTTGGAGGATTTTGTATAGGAAAAGT |
| | | TGAACCTTCTTGGCTTGGTCCTTTATTTCCAGATAAGACTTCTAAT |
| | | TTAAGGAAACAAACAAATATAGCATCTACACTAGCAAGAATGGT |
| | | GATCAGATATCAGATGAAAAGTGCTGTGGAAGAAGGAACAGCCT |
| | | CAGGCAGCGATGGAAATTTTTCTGAAGATGTGCTGTCTAAATTTG |
| | | ATGAATGGACCTTTATTCCTGACTCTTCTATGGACAGTGTGTTTGC |
| | | TCAAAGTGATGACCTGGATAGTGAAGGAAGTGAAGGCTCATTTC |
| | | TTGTGAAAAAGAAATCTAATTCAATTAGTGTAGGAGAATTTTACC |
| | | GAGATGCCGTATTACAGCGTTGCTCACCAAATTTGCAAAGACATT |
| | | CCAATTCCTTGGGGCCCATTTTTGATCATGAAGATTTACTGAAGC |
| | | GAAAAAGAAAAATATTATCTTCAGATGATTCACTCAGGTCATCA |
| | | AAACTTCAATCCCATATGAGGCATTCAGACAGCATTTCTTCTCTG |
| | | GCTTCTGAGAGAGAATATATTACATCACTAGACCTTTCAGCAAAT |
| | | GAACTAAGAGATATTGATGCCCTAAGCCAGAAATGCTGTATAAG |
| | | TGTTCATTTGGAGCATCTTGAAAAGCTGGAGCTTCACCAGAATGC |
| | | ACTCACGAGCTTTCCACAACAGCTATGTGAAACTCTGAAGAGTTT |
| | | GACACATTTGGACTTGCACAGTAATAAATTTACATCATTTCCTTC |
| | | TTATTTGTTGAAAATGAGTTGTATTGCTAATCTTGATGTCTCTCGA |
| | | AATGACATTGGACCCTCAGTGGTTTTAGATCCTACAGTGAAATGT |
| | | CCAACTCTGAAACAGTTTAACCTGTCATATAACCAGCTGTCTTTT |
| | | GTACCTGAGAACCTCACTGATGTGGTAGAGAAACTGGAGCAGCT |
| | | CATTTTAGAAGGAAATAAAATATCAGGGATATGCTCCCCCTTGAG |
| | | ACTGAAGGAACTGAAGATTTTAAACCTTAGTAAGAACCACATTTC |
| | | ATCCCTATCAGAGAACTTTCTTGAGGCTTGTCCTAAAGTGGAGAG |
| | | TTTCAGTGCCAGAATGAATTTTCTTGCTGCTATGCCTTTCTTGCCT |
| | | CCTTCTATGACAATCCTAAAATTATCTCAGAACAAATTTTCCTGT |
| | | ATTCCAGAAGCAATTTTAAATCTTCCACACTTGCGGTCTTTAGAT |
| | | ATGAGCAGCAATGATATTCAGTACCTACCAGGTCCCGCACACTG |
| | | GAAATCTTTGAACTTAAGGGAACTCTTATTTAGCCATAATCAGAT |
| | | CAGCATCTTGGACTTGAGTGAAAAAGCATATTTATGGTCTAGAGT |
| | | AGAGAAACTGCATCTTTCTCACAATAAACTGAAAGAGATTCCTCC |
| | | TGAGATTGGCTGTCTTGAAAATCTGACATCTCTGGATGTCAGTTA |
| | | CAACTTGGAACTAAGATCCTTTCCCAATGAAATGGGGAAATTAA |
| | | GCAAAATATGGGATCTTCCTTTGGATGAACTGCATCTTAACTTTG |
| | | ATTTTAAACATATAGGATGTAAAGCCAAAGACATCATAAGGTTTC |
| | | TTCAACAGCGATTAAAAAAGGCTGTGCCTTATAACCGAATGAAA |
| | | CTTATGATTGTGGGAAATACTGGGAGTGGTAAAACCACCTTATTG |
| | | CAGCAATTAATGAAAACCAAGAAATCAGATCTTGGAATGCAAAG |
| | | TGCCACAGTTGGCATAGATGTGAAAGACTGGCCTATCCAAATAA |
| | | GAGACAAAAGAAAGAGAGATCTCGTCCTAAATGTGTGGGATTTT |
| | | GCAGGTCGTGAGGAATTCTATAGTACTCATCCCCATTTTATGACG |
| | | CAGCGAGCATTGTACCTTGCTGTCTATGACCTCAGCAAGGGACAG |
| | | GCTGAAGTTGATGCCATGAAGCCTTGGCTCTTCAATATAAAGGCT |
| | | CGCGCTTCTTCTTCCCCTGTGATTCTCGTTGGCACACATTTGGATG |
| | | TTTCTGATGAGAAGCAACGCAAAGCCTGCATGAGTAAAATCACC |
| | | AAGGAACTCCTGAATAAGCGAGGGTTCCCTGCCATACGAGATTA |
| | | CCACTTTGTGAATGCCACCGAGGAATCTGATGCTTTGGCAAAACT |
| | | TCGGAAAACCATCATAAACGAGAGCCTTAATTTCAAGATCCGAG |
| | | ATCAGCTTGTTGTTGGACAGCTGATTCCAGACTGCTATGTAGAAC |
| | | TTGAAAAAATCATTTTATCGGAGCGTAAAAATGTGCCAATTGAAT |
| | | TTCCCGTAATTGACCGGAAACGATTATTACAACTAGTGAGAGAA |
| | | AATCAGCTGCAGTTAGATGAAAATGAGCTTCCTCACGCAGTTCAC |
| | | TTTCTAAATGAATCAGGAGTCCTTCTTCATTTTCAAGACCCAGCA |
| | | CTGCAGTTAAGTGACTTGTACTTTGTGGAACCCAAGTGGCTTTGT |
| | | AAAATCATGGCACAGATTTTGACAGTGAAAGTGGAAGGTTGTCC |
| | | AAAACACCCTAAGGGCATTATTTCGCGTAGAGATGTGGAAAAAT |
| | | TTCTTTCAAAAAAAAGGAAATTTCCAAAGAACTACATGTCACAGT |
| | | ATTTTAAGCTCCTAGAAAAATTCCAGATTGCTTTGCCAATAGGAG |
| | | AAGAATATTTGCTGGTTCCAAGCAGTTTGTCTGACCACAGGCCTG |
| | | TGATAGAGCTTCCCCATTGTGAGAACTCTGAAATTATCATCCGAC |
| | | TATATGAAATGCCTTATTTTCCAATGGGATTTTGGTCAAGATTAA |
| | | TCAATCGATTACTTGAGATTTCACCTTACATGCTTTCAGGGAGAG |
| | | AACGAGCACTTCGCCCAAACAGAATGTATTGGCGACAAGGCATT |
| | | TACTTAAATTGGTCTCCTGAAGCTTATTGTCTGGTAGGATCTGAA |
| | | GTCTTAGACAATCATCCAGAGAGTTTCTTAAAAATTACAGTTCCT |
| | | TCTTGTAGAAAAGGCTGTATTCTTTTGGGCCAAGTTGTGGACCAC |
| | | ATTGATTCTCTCATGGAAGAATGGTTTCCTGGGTTGCTGGAGATT |
| | | GATATTTGTGGTGAAGGAGAAACTCTGTTGAAGAAATGGGCATT |
| | | ATATAGTTTTAATGATGGTGAAGAACATCAAAAAATCTTACTTGA |
| | | TGACTTGATGAAGAAAGCAGAGGAAGGAGATCTCTTAGTAAATC |
| | | CAGATCAACCAAGGCTCACCATTCCAATATCTCAGATTGCCCCTG |
| | | ACTTGATTTTGGCTGACCTGCCTAGAAATATTATGTTGAATAATG |
| | | ATGAGTTGGAATTTGAACAAGCTCCAGAGTTTCTCCTAGGTGATG |
| | | GCAGTTTTGGATCAGTTTACCGAGCAGCCTATGAAGGAGAAGAA |
| | | GTGGCTGTGAAGATTTTTAATAAACATACATCACTCAGGCTGTTA |
| | | AGACAAGAGCTTGTGGTGCTTTGCCACCTCCACCACCCCAGTTTG |
| | | ATATCTTTGCTGGCAGCTGGGATTCGTCCCCGGATGTTGGTGATG |
| | | GAGTTAGCCTCCAAGGGTTCCTTGGATCGCCTGCTTCAGCAGGAC |
| | | AAAGCCAGCCTCACTAGAACCCTACAGCACAGGATTGCACTCCA |
| | | CGTAGCTGATGGTTTGAGATACCTCCACTCAGCCATGATTATATA |
| | | CCGAGACCTGAAACCCCACAATGTGCTGCTTTTCACACTGTATCC |
| | | CAATGCTGCCATCATTGCAAAGATTGCTGACTACGGCATTGCTCA |
| | | GTACTGCTGTAGAATGGGGATAAAAACATCAGAGGGCACACCAG |
| | | GGTTTCGTGCACCTGAAGTTGCCAGAGGAAATGTCATTTATAACC |
| | | AACAGGCTGATGTTTATTCATTTGGTTTACTACTCTATGACATTTT |
| | | GACAACTGGAGGTAGAATAGTAGAGGGTTTGAAGTTTCCAAATG |
| | | AGTTTGATGAATTAGAAATACAAGGAAAATTACCTGATCCAGTT |
| | | AAAGAATATGGTTGTGCCCCATGGCCTATGGTTGAGAAATTAATT |
| | | AAACAGTGTTTGAAAGAAAATCCTCAAGAAAGGCCTACTTCTGC |
| | | CCAGGTCTTTGACATTTTGAATTCAGCTGAATTAGTCTGTCTGAC |
| | | GAGACGCATTTTATTACCTAAAAACGTAATTGTTGAATGCATGGT |
| | | TGCTACACATCACAACAGCAGGAATGCAAGCATTTGGCTGGGCT |
| | | GTGGGCACACCGACAGAGGACAGCTCTCATTTCTTGACTTAAATA |
| | | CTGAAGGATACACTTCTGAGGAAGTTGCTGATAGTAGAATATTGT |
| | | GCTTAGCCTTGGTGCATCTTCCTGTTGAAAAGGAAAGCTGGATTG |
| | | TGTCTGGGACACAGTCTGGTACTCTCCTGGTCATCAATACCGAAG |
| | | ATGGGAAAAAGAGACATACCCTAGAAAAGATGACTGATTCTGTC |
| | | ACTTGTTTGTATTGCAATTCCTTTTCCAAGCAAAGCAAACAAAAA |
| | | AATTTTCTTTTGGTTGGAACCGCTGATGGCAAGTTAGCAATTTTT |
| | | GAAGATAAGACTGTTAAGCTTAAAGGAGCTGCTCCTTTGAAGAT |
| | | ACTAAATATAGGAAATGTCAGTACTCCATTGATGTGTTTGAGTGA |
| | | ATCCACAAATTCAACGGAAAGAAATGTAATGTGGGGAGGATGTG |
| | | GCACAAAGATTTTCTCCTTTTCTAATGATTTCACCATTCAGAAACT |
| | | CATTGAGACAAGAACAAGCCAACTGTTTTCTTATGCAGCTTTCAG |
| | | TGATTCCAACATCATAACAGTGGTGGTAGACACTGCTCTCTATAT |
| | | TGCTAAGCAAAATAGCCCTGTTGTGGAAGTGTGGGATAAGAAAA |
| | | CTGAAAAACTCTGTGGACTAATAGACTGCGTGCACTTTTTAAGGG |
| | | AGGTAATGGTAAAAGAAAACAAGGAATCAAAACACAAAATGTCT |
| | | TATTCTGGGAGAGTGAAAACCCTCTGCCTTCAGAAGAACACTGCT |
| | | CTTTGGATAGGAACTGGAGGAGGCCATATTTTACTCCTGGATCTT |
| | | TCAACTCGTCGACTTATACGTGTAATTTACAACTTTTGTAATTCGG |
| | | TCAGAGTCATGATGACAGCACAGCTAGGAAGCCTTAAAAATGTC |
| | | ATGCTGGTATTGGGCTACAACCGGAAAAATACTGAAGAGATACA |
| | | ATCTTGCTTGACCGTTTGGGACATCAATCTTCCACATGAAGTGCA |
| | | AAATTTAGAAAAACACATTGAAGTGAGAAAAGAATTAGCTGAAA |
| | | AAATGAGACGAACATCTGTTGAGTAAGAGAGAAATAGGAATTGT |
| | | CTTTGGATAGGAAAATTATTCTCTCCTCTTGTAAATATTTATTTTA |
| | | AAAATGTTCACATGGAAAGGGTACTCACATTTTTTGAAATAGCTC |
| | | GTGTGTATGAAGGAATGTTATTATTTTTAATTTAAATATATGTAA |
| | | AAATACTTACCAGTAAATGTGTATTTTAAAGAACTATTTAAAACA |
| | | CAATGTTATATTTCTTATAAATACCAGTTACTTTCGTTCATTAATT |
| | | AATGAAAATAAATCTGTGAAGTACCTAATTTAAGTACTCATACTA |
| | | AAATTTATAAGGCCGATAATTTTTTGTTTTCTTGTCTGTAATGGAG |
| | | GTAAACTTTATTTTAAATTCTGTGCTTAAGACAGGACTATTGCTT |
| | | GTCGATTTTTCTAGAAATCTGCACGGTATAATGAAAATATTAAGA |
| | | CAGTTTCCCATGTAATGTATTCCTTCTTAGATTGCATCGAAATGC |
| | | ACTATCATATATGCTTGTAAATATTCAAATGAATTTGCACTAATA |
| | | AAGTCCTTTGTTGGTATGTGAATTCTCTTTGTTGCTGTTGCAAACA |
| | | GTGCATCTTACACAACTTCACTCAATTCAAAAGAAAACTCCATTA |
| | | AAAGTACTAATGAAAAAACATGACATACTGTCAAAGTCCTCATA |
| | | TCTAGGAAAGACACAGAAACTCTCTTTGTCACAGAAACTCTCTGT |
| | | GTCTTTCCTAGACATAATAGAGTTGTTTTTCAACTCTATGTTTGAA |
| | | TGTGGATACCCTGAATTTTGTATAATTAGTGTAAATACAGTGTTC |
| | | AGTCCTTCAAGTGATATTTTTATTTTTTTATTCATACCACTAGCTA |
| | | CTTGTTTTCTAATCTGCTTCATTCTAATGCTTATATTCATCTTTTCC |
| | | CTAAATTTGTGATGCTGCAGATCCTACATCATTCAGATAGAAACC |
| | | TTTTTTTTTTTCAGAATTATAGAATTCCACAGCTCCTACCAAGACC |
| | | ATGAGGATAAATATCTAACACTTTTCAGTTGCTGAAGGAGAAAG |
| | | GAGCTTTAGTTATGATGGATAAAAATATCTGCCACCCTAGGCTTC |
| | | CAAATTATACTTAAATTGTTTACATAGCTTACCACAATAGGAGTA |
| | | TCAGGGCCAAATACCTATGTAATAATTTGAGGTCATTTCTGCTTT |
| | | AGGAAAAGTACTTTCGGTAAATTCTTTGGCCCTGACCAGTATTCA |
| | | TTATTTCAGATAATTCCCTGTGATAGGACAACTAGTACATTTAAT |
| | | ATTCTCAGAACTTATGGCATTTTACTATGTGAAAACTTTAAATTT |
| | | ATTTATATTAAGGGTAATCAAATTCTTAAAGATGAAAGATTTTCT |
| | | GTATTTTAAAGGAAGCTATGCTTTAACTTGTTATGTAATTAACAA |
| | | AAAAATCATATATAATAGAGCTCTTTGTTCCAGTGTTATCTCTTTC |
| | | ATTGTTACTTTGTATTTGCAATTTTTTTTACCAAAGACAAATTAAA |
| | | AAAATGAATACCATATTTAAATGGAATAATAAAGGTTTTTTAAA |
| 7 | X2 | CCTGAGTGGGGGAGGAGGAAGCCGAGCAGGAGGGCTCCGGAGA |
| | | GGGAGGGCAACGCGGGGCGGGGAGCTGCCTCCTTCCTCATAAAC |
| | | AGGCGGGCGTGGGCGCCGATGGGGCCCGCGGGGAGCGCTGGCTG |
| | | CGGGCGGTGAGCTGAGCTCGCCCCCGGGGAGCTGTGGCCGGCGC |
| | | CCCTGCCGGTTCCCTGAGCAGCGGACGTTCATGCTGGGAGGGCG |
| | | GCGGGTTGGAAGCAGGTGCCACCATGGCTAGTGGCAGCTGTCAG |
| | | GGGTGCGAAGAGGACGAGGAAACTCTGAAGAAGTTGATAGTCAG |
| | | GCTGAACAATGTCCAGGAAGGAAAACAGATAGAAACGCTGGTCC |
| | | AAATCCTGGAGGATCTGCTGGTGTTCACGTACTCCGAGCGCGCCT |
| | | CCAAGTTATTTCAAGGCAAAAATATCCATGTGCCTCTGTTGATCG |
| | | TCTTGGACTCCTATATGAGAGTCGCGAGTGTGCAGCAGGTGGGTT |
| | | GGTCACTTCTGTGCAAATTAATAGAAGTCTGTCCAGGTACAATGC |
| | | AAAGCTTAATGGGACCCCAGGATGTTGGAAATGATTGGGAAGTC |
| | | CTTGGTGTTCACCAATTGATTCTTAAAATGCTAACAGTTCATAAT |
| | | GCCAGTGTAAACTTGTCAGTGATTGGACTGAAGACCTTAGATCTC |
| | | CTCCTAACTTCAGGTAAAATCACCTTGCTGATATTGGATGAAGAA |
| | | AGTGATATTTTCATGTTAATTTTTGATGCCATGCACTCATTTCCAG |
| | | CCAATGATGAAGTCCAGAAACTTGGATGCAAAGCTTTACATGTG |
| | | CTGTTTGAGAGAGTCTCAGAGGAGCAACTGACTGAATTTGTTGAG |
| | | AACAAAGATTATATGATATTGTTAAGTGCGTTAACAAATTTTAAA |
| | | GATGAAGAGGAAATTGTGCTTCATGTGCTGCATTGTTTACATTCC |
| | | CTAGCGATTCCTTGCAATAATGTGGAAGTCCTCATGAGTGGCAAT |
| | | GTCAGGTGTTATAATATTGTGGTGGAAGCTATGAAAGCATTCCCT |
| | | ATGAGTGAAAGAATTCAAGAAGTGAGTTGCTGTTTGCTCCATAG |
| | | GCTTACATTAGGTAATTTTTTCAATATCCTGGTATTAAACGAAGT |
| | | CCATGAGTTTGTGGTGAAAGCTGTGCAGCAGTACCCAGAGAATG |
| | | CAGCATTGCAGATCTCAGCGCTCAGCTGTTTGGCCCTCCTCACTG |
| | | AGACTATTTTCTTAAATCAAGATTTAGAGGAAAAGAATGAGAAT |
| | | CAAGAGAATGATGATGAGGGGGAAGAAGATAAATTGTTTTGGCT |
| | | GGAAGCCTGTTACAAAGCATTAACGTGGCATAGAAAGAACAAGC |
| | | ACGTGCAGGAGGCCGCATGCTGGGCACTAAATAATCTCCTTATGT |
| | | ACCAAAACAGTTTACATGAGAAGATTGGAGATGAAGATGGCCAT |
| | | TTCCCAGCTCATAGGGAAGTGATGCTCTCCATGCTGATGCATTCT |
| | | TCATCAAAGGAAGTTTTCCAGGCATCTGCGAATGCATTGTCAACT |
| | | CTCTTAGAACAAAATGTTAATTTCAGAAAAATACTGTTATCAAAA |
| | | GGAATACACCTGAATGTTTTGGAGTTAATGCAGAAGCATATACAT |
| | | TCTCCTGAAGTGGCTGAAAGTGGCTGTAAAATGCTAAATCATCTT |
| | | TTTGAAGGAAGCAACACTTCCCTGGATATAATGGCAGCAGTGGT |
| | | CCCCAAAATACTAACAGTTATGAAACGTCATGAGACATCATTACC |
| | | AGTGCAGCTGGAGGCGCTTCGAGCTATTTTACATTTTATAGTGCC |
| | | TGGCATGCCAGAAGAATCCAGGGAGGATACAGAATTTCATCATA |
| | | AGCTAAATATGGTTAAAAAACAGTGTTTCAAGAATGATATTCAC |
| | | AAACTGGTCCTAGCAGCTTTGAACAGGTTCATTGGAAATCCTGGG |
| | | ATTCAGAAATGTGGATTAAAAGTAATTTCTTCTATTGTACATTTTC |
| | | CTGATGCATTAGAGATGTTATCCCTGGAAGGTGCTATGGATTCAG |
| | | TGCTTCACACACTGCAGATGTATCCAGATGACCAAGAAATTCAGT |
| | | GTCTGGGTTTAAGTCTTATAGGATACTTGATTACAAAGAAGAATG |
| | | TGTTCATAGGAACTGGACATCTGCTGGCAAAAATTCTGGTTTCCA |
| | | GCTTATACCGATTTAAGGATGTTGCTGAAATACAGACTAAAGGAT |
| | | TTCAGACAATCTTAGCAATCCTCAAATTGTCAGCATCTTTTTCTAA |
| | | GCTGCTGGTGCATCATTCATTTGACTTAGTAATATTCCATCAAAT |
| | | GTCTTCCAATATCATGGAACAAAAGGATCAACAGTTTCTAAACCT |
| | | CTGTTGCAAGTGTTTTGCAAAAGTAGCTATGGATGATTACTTAAA |
| | | AAATGTGATGCTAGAGAGAGCGTGTGATCAGAATAACAGCATCA |
| | | TGGTTGAATGCTTGCTTCTATTGGGAGCAGATGCCAATCAAGCAA |
| | | AGGAGGGATCTTCTTTAATTTGTCAGGTATGTGAGAAAGAGAGC |
| | | AGTCCCAAATTGGTGGAACTCTTACTGAATAGTGGATCTCGTGAA |
| | | CAAGATGTACGAAAAGCGTTGACGATAAGCATTGGGAAAGGTGA |
| | | CAGCCAGATCATCAGCTTGCTCTTAAGGAGGCTGGCCCTGGATGT |
| | | GGCCAACAATAGCATTTGCCTTGGAGGATTTTGTATAGGAAAAGT |
| | | TGAACCTTCTTGGCTTGGTCCTTTATTTCCAGATAAGACTTCTAAT |
| | | TTAAGGAAACAAACAAATATAGCATCTACACTAGCAAGAATGGT |
| | | GATCAGATATCAGATGAAAAGTGCTGTGGAAGAAGGAACAGCCT |
| | | CAGGCAGCGATGGAAATTTTTCTGAAGATGTGCTGTCTAAATTTG |
| | | ATGAATGGACCTTTATTCCTGACTCTTCTATGGACAGTGTGTTTGC |
| | | TCAAAGTGATGACCTGGATAGTGAAGGAAGTGAAGGCTCATTTC |
| | | TTGTGAAAAAGAAATCTAATTCAATTAGTGTAGGAGAATTTTACC |
| | | GAGATGCCGTATTACAGCGTTGCTCACCAAATTTGCAAAGACATT |
| | | CCAATTCCTTGGGGCCCATTTTTGATCATGAAGATTTACTGAAGC |
| | | GAAAAAGAAAAATATTATCTTCAGATGATTCACTCAGGTCATCA |
| | | AAACTTCAATCCCATATGAGGCATTCAGACAGCATTTCTTCTCTG |
| | | GCTTCTGAGAGAGAATATATTACATCACTAGACCTTTCAGCAAAT |
| | | GAACTAAGAGATATTGATGCCCTAAGCCAGAAATGCTGTATAAG |
| | | TGTTCATTTGGAGCATCTTGAAAAGCTGGAGCTTCACCAGAATGC |
| | | ACTCACGAGCTTTCCACAACAGCTATGTGAAACTCTGAAGAGTTT |
| | | GACACATTTGGACTTGCACAGTAATAAATTTACATCATTTCCTTC |
| | | TTATTTGTTGAAAATGAGTTGTATTGCTAATCTTGATGTCTCTCGA |
| | | AATGACATTGGACCCTCAGTGGTTTTAGATCCTACAGTGAAATGT |
| | | CCAACTCTGAAACAGTTTAACCTGTCATATAACCAGCTGTCTTTT |
| | | GTACCTGAGAACCTCACTGATGTGGTAGAGAAACTGGAGCAGCT |
| | | CATTTTAGAAGGAAATAAAATATCAGGGATATGCTCCCCCTTGAG |
| | | ACTGAAGGAACTGAAGATTTTAAACCTTAGTAAGAACCACATTTC |
| | | ATCCCTATCAGAGAACTTTCTTGAGGCTTGTCCTAAAGTGGAGAG |
| | | TTTCAGTGCCAGAATGAATTTTCTTGCTGCTATGCCTTTCTTGCCT |
| | | CCTTCTATGACAATCCTAAAATTATCTCAGAACAAATTTTCCTGT |
| | | ATTCCAGAAGCAATTTTAAATCTTCCACACTTGCGGTCTTTAGAT |
| | | ATGAGCAGCAATGATATTCAGTACCTACCAGGTCCCGCACACTG |
| | | GAAATCTTTGAACTTAAGGGAACTCTTATTTAGCCATAATCAGAT |
| | | CAGCATCTTGGACTTGAGTGAAAAAGCATATTTATGGTCTAGAGT |
| | | AGAGAAACTGCATCTTTCTCACAATAAACTGAAAGAGATTCCTCC |
| | | TGAGATTGGCTGTCTTGAAAATCTGACATCTCTGGATGTCAGTTA |
| | | CAACTTGGAACTAAGATCCTTTCCCAATGAAATGGGGAAATTAA |
| | | GCAAAATATGGGATCTTCCTTTGGATGAACTGCATCTTAACTTTG |
| | | ATTTTAAACATATAGGATGTAAAGCCAAAGACATCATAAGGTTTC |
| | | TTCAACAGCGATTAAAAAAGGCTGTGCCTTATAACCGAATGAAA |
| | | CTTATGATTGTGGGAAATACTGGGAGTGGTAAAACCACCTTATTG |
| | | CAGCAATTAATGAAAACCAAGAAATCAGATCTTGGAATGCAAAG |
| | | TGCCACAGTTGGCATAGATGTGAAAGACTGGCCTATCCAAATAA |
| | | GAGACAAAAGAAAGAGAGATCTCGTCCTAAATGTGTGGGATTTT |
| | | GCAGGTCGTGAGGAATTCTATAGTACTCATCCCCATTTTATGACG |
| | | CAGCGAGCATTGTACCTTGCTGTCTATGACCTCAGCAAGGGACAG |
| | | GCTGAAGTTGATGCCATGAAGCCTTGGCTCTTCAATATAAAGGCT |
| | | CGCGCTTCTTCTTCCCCTGTGATTCTCGTTGGCACACATTTGGATG |
| | | TTTCTGATGAGAAGCAACGCAAAGCCTGCATGAGTAAAATCACC |
| | | AAGGAACTCCTGAATAAGCGAGGGTTCCCTGCCATACGAGATTA |
| | | CCACTTTGTGAATGCCACCGAGGAATCTGATGCTTTGGCAAAACT |
| | | TCGGAAAACCATCATAAACGAGAGCCTTAATTTCAAGATCCGAG |
| | | ATCAGCTTGTTGTTGGACAGCTGATTCCAGACTGCTATGTAGAAC |
| | | TTGAAAAAATCATTTTATCGGAGCGTAAAAATGTGCCAATTGAAT |
| | | TTCCCGTAATTGACCGGAAACGATTATTACAACTAGTGAGAGAA |
| | | AATCAGCTGCAGTTAGATGAAAATGAGCTTCCTCACGCAGTTCAC |
| | | TTTCTAAATGAATCAGGAGTCCTTCTTCATTTTCAAGACCCAGCA |
| | | CTGCAGTTAAGTGACTTGTACTTTGTGGAACCCAAGTGGCTTTGT |
| | | AAAATCATGGCACAGATTTTGACAGTGAAAGTGGAAGGTTGTCC |
| | | AAAACACCCTAAGGGCATTATTTCGCGTAGAGATGTGGAAAAAT |
| | | TTCTTTCAAAAAAAAGGAAATTTCCAAAGAACTACATGTCACAGT |
| | | ATTTTAAGCTCCTAGAAAAATTCCAGATTGCTTTGCCAATAGGAG |
| | | AAGAATATTTGCTGGTTCCAAGCAGTTTGTCTGACCACAGGCCTG |
| | | TGATAGAGCTTCCCCATTGTGAGAACTCTGAAATTATCATCCGAC |
| | | TATATGAAATGCCTTATTTTCCAATGGGATTTTGGTCAAGATTAA |
| | | TCAATCGATTACTTGAGATTTCACCTTACATGCTTTCAGGGAGAG |
| | | AACGAGCACTTCGCCCAAACAGAATGTATTGGCGACAAGGCATT |
| | | TACTTAAATTGGTCTCCTGAAGCTTATTGTCTGGTAGGATCTGAA |
| | | GTCTTAGACAATCATCCAGAGAGTTTCTTAAAAATTACAGTTCCT |
| | | TCTTGTAGAAAAGGCTGTATTCTTTTGGGCCAAGTTGTGGACCAC |
| | | ATTGATTCTCTCATGGAAGAATGGTTTCCTGGGTTGCTGGAGATT |
| | | GATATTTGTGGTGAAGGAGAAACTCTGTTGAAGAAATGGGCATT |
| | | ATATAGTTTTAATGATGGTGAAGAACATCAAAAAATCTTACTTGA |
| | | TGACTTGATGAAGAAAGCAGAGGAAGGAGATCTCTTAGTAAATC |
| | | CAGATCAACCAAGGCTCACCATTCCAATATCTCAGATTGCCCCTG |
| | | ACTTGATTTTGGCTGACCTGCCTAGAAATATTATGTTGAATAATG |
| | | ATGAGTTGGAATTTGAACAAGCTCCAGAGTTTCTCCTAGGTGATG |
| | | GCAGTTTTGGATCAGTTTACCGAGCAGCCTATGAAGGAGAAGAA |
| | | GTGGCTGTGAAGATTTTTAATAAACATACATCACTCAGGCTGTTA |
| | | AGACAAGAGCTTGTGGTGCTTTGCCACCTCCACCACCCCAGTTTG |
| | | ATATCTTTGCTGGCAGCTGGGATTCGTCCCCGGATGTTGGTGATG |
| | | GAGTTAGCCTCCAAGGGTTCCTTGGATCGCCTGCTTCAGCAGGAC |
| | | AAAGCCAGCCTCACTAGAACCCTACAGCACAGGATTGCACTCCA |
| | | CGTAGCTGATGGTTTGAGATACCTCCACTCAGCCATGATTATATA |
| | | CCGAGACCTGAAACCCCACAATGTGCTGCTTTTCACACTGTATCC |
| | | CAATGCTGCCATCATTGCAAAGATTGCTGACTACGGCATTGCTCA |
| | | GTACTGCTGTAGAATGGGGATAAAAACATCAGAGGGCACACCAG |
| | | GGTTTCGTGCACCTGAAGTTGCCAGAGGAAATGTCATTTATAACC |
| | | AACAGGCTGATGTTTATTCATTTGGTTTACTACTCTATGACATTTT |
| | | GACAACTGGAGGTAGAATAGTAGAGGGTTTGAAGTTTCCAAATG |
| | | AGTTTGATGAATTAGAAATACAAGGAAAATTACCTGATCCAGTT |
| | | AAAGAATATGGTTGTGCCCCATGGCCTATGGTTGAGAAATTAATT |
| | | AAACAGTGTTTGAAAGAAAATCCTCAAGAAAGGCCTACTTCTGC |
| | | CCAGGTCTTTGACATTTTGAATTCAGCTGAATTAGTCTGTCTGAC |
| | | GAGACGCATTTTATTACCTAAAAACGTAATTGTTGAATGCATGGT |
| | | TGCTACACATCACAACAGCAGGAATGCAAGCATTTGGCTGGGCT |
| | | GTGGGCACACCGACAGAGGACAGCTCTCATTTCTTGACTTAAATA |
| | | CTGAAGGATACACTTCTGAGGAAGTTGCTGATAGTAGAATATTGT |
| | | GCTTAGCCTTGGTGCATCTTCCTGTTGAAAAGGAAAGCTGGATTG |
| | | TGTCTGGGACACAGTCTGGTACTCTCCTGGTCATCAATACCGAAG |
| | | ATGGGAAAAAGAGACATACCCTAGAAAAGATGACTGATTCTGTC |
| | | ACTTGTTTGTATTGCAATTCCTTTTCCAAGCAAAGCAAACAAAAA |
| | | AATTTTCTTTTGGTTGGAACCGCTGATGGCAAGTTAGCAATTTTT |
| | | GAAGATAAGACTGTTAAGCTTAAAGGAGCTGCTCCTTTGAAGAT |
| | | ACTAAATATAGGAAATGTCAGTACTCCATTGATGTGTTTGAGTGA |
| | | ATCCACAAATTCAACGGAAAGAAATGTAATGTGGGGAGGATGTG |
| | | GCACAAAGATTTTCTCCTTTTCTAATGATTTCACCATTCAGAAACT |
| | | CATTGAGACAAGAACAAGCCAACTGTTTTCTTATGCAGCTTTCAG |
| | | TGATTCCAACATCATAACAGTGGTGGTAGACACTGCTCTCTATAT |
| | | TGCTAAGCAAAATAGCCCTGTTGTGGAAGTGTGGGATAAGAAAA |
| | | CTGAAAAACTCTGTGGACTAATAGACTGCGTGCACTTTTTAAGGG |
| | | AGGTAATGGTAAAAGAAAACAAGGAATCAAAACACAAAATGTCT |
| | | TATTCTGGGAGAGTGAAAACCCTCTGCCTTCAGAAGAACACTGCT |
| | | CTTTGGATAGGAACTGGAGGAGGCCATATTTTACTCCTGGATCTT |
| | | TCAACTCGTCGACTTATACGTGTAATTTACAACTTTTGTAATTCGG |
| | | TCAGAGTCATGATGACAGCACAGCTAGAGATACAATCTTGCTTG |
| | | ACCGTTTGGGACATCAATCTTCCACATGAAGTGCAAAATTTAGAA |
| | | AAACACATTGAAGTGAGAAAAGAATTAGCTGAAAAAATGAGAC |
| | | GAACATCTGTTGAGTAAGAGAGAAATAGGAATTGTCTTTGGATA |
| | | GGAAAATTATTCTCTCCTCTTGTAAATATTTATTTTAAAAATGTTC |
| | | ACATGGAAAGGGTACTCACATTTTTTGAAATAGCTCGTGTGTATG |
| | | AAGGAATGTTATTATTTTTAATTTAAATATATGTAAAAATACTTA |
| | | CCAGTAAATGTGTATTTTAAAGAACTATTTAAAACACAATGTTAT |
| | | ATTTCTTATAAATACCAGTTACTTTCGTTCATTAATTAATGAAAAT |
| | | AAATCTGTGAAGTACCTAATTTAAGTACTCATACTAAAATTTATA |
| | | AGGCCGATAATTTTTTGTTTTCTTGTCTGTAATGGAGGTAAACTTT |
| | | ATTTTAAATTCTGTGCTTAAGACAGGACTATTGCTTGTCGATTTTT |
| | | CTAGAAATCTGCACGGTATAATGAAAATATTAAGACAGTTTCCCA |
| | | TGTAATGTATTCCTTCTTAGATTGCATCGAAATGCACTATCATAT |
| | | ATGCTTGTAAATATTCAAATGAATTTGCACTAATAAAGTCCTTTG |
| | | TTGGTATGTGAATTCTCTTTGTTGCTGTTGCAAACAGTGCATCTTA |
| | | CACAACTTCACTCAATTCAAAAGAAAACTCCATTAAAAGTACTA |
| | | ATGAAAAAACATGACATACTGTCAAAGTCCTCATATCTAGGAAA |
| | | GACACAGAAACTCTCTTTGTCACAGAAACTCTCTGTGTCTTTCCT |
| | | AGACATAATAGAGTTGTTTTTCAACTCTATGTTTGAATGTGGATA |
| | | CCCTGAATTTTGTATAATTAGTGTAAATACAGTGTTCAGTCCTTC |
| | | AAGTGATATTTTTATTTTTTTATTCATACCACTAGCTACTTGTTTT |
| | | CTAATCTGCTTCATTCTAATGCTTATATTCATCTTTTCCCTAAATT |
| | | TGTGATGCTGCAGATCCTACATCATTCAGATAGAAACCTTTTTTTT |
| | | TTTCAGAATTATAGAATTCCACAGCTCCTACCAAGACCATGAGGA |
| | | TAAATATCTAACACTTTTCAGTTGCTGAAGGAGAAAGGAGCTTTA |
| | | GTTATGATGGATAAAAATATCTGCCACCCTAGGCTTCCAAATTAT |
| | | ACTTAAATTGTTTACATAGCTTACCACAATAGGAGTATCAGGGCC |
| | | AAATACCTATGTAATAATTTGAGGTCATTTCTGCTTTAGGAAAAG |
| | | TACTTTCGGTAAATTCTTTGGCCCTGACCAGTATTCATTATTTCAG |
| | | ATAATTCCCTGTGATAGGACAACTAGTACATTTAATATTCTCAGA |
| | | ACTTATGGCATTTTACTATGTGAAAACTTTAAATTTATTTATATTA |
| | | AGGGTAATCAAATTCTTAAAGATGAAAGATTTTCTGTATTTTAAA |
| | | GGAAGCTATGCTTTAACTTGTTATGTAATTAACAAAAAAATCATA |
| | | TATAATAGAGCTCTTTGTTCCAGTGTTATCTCTTTCATTGTTACTT |
| | | TGTATTTGCAATTTTTTTTACCAAAGACAAATTAAAAAAATGAAT |
| | | ACCATATTTAAATGGAATAATAAAGGTTTTTTAAACCTGAGTGGG |
| | | GGAGGAGGAAGCCGAGCAGGAGGGCTCCGGAGAGGGAGGGCAA |
| | | CGCGGGGCGGGGAGCTGCCTCCTTCCTCATAAACAGGCGGGCGT |
| | | GGGCGCCGATGGGGCCCGCGGGGAGCGCTGGCTGCGGGCGGTGA |
| | | GCTGAGCTCGCCCCCGGGGAGCTGTGGCCGGCGCCCCTGCCGGTT |
| | | CCCTGAGCAGCGGACGTTCATGCTGGGAGGGCGGCGGGTTGGAA |
| | | GCAGGTGCCACCATGGCTAGTGGCAGCTGTCAGGGGTGCGAAGA |
| | | GGACGAGGAAACTCTGAAGAAGTTGATAGTCAGGCTGAACAATG |
| | | TCCAGGAAGGAAAACAGATAGAAACGCTGGTCCAAATCCTGGAG |
| | | GATCTGCTGGTGTTCACGTACTCCGAGCGCGCCTCCAAGTTATTT |
| | | CAAGGCAAAAATATCCATGTGCCTCTGTTGATCGTCTTGGACTCC |
| | | TATATGAGAGTCGCGAGTGTGCAGCAGGTGGGTTGGTCACTTCTG |
| | | TGCAAATTAATAGAAGTCTGTCCAGGTACAATGCAAAGCTTAAT |
| | | GGGACCCCAGGATGTTGGAAATGATTGGGAAGTCCTTGGTGTTC |
| | | ACCAATTGATTCTTAAAATGCTAACAGTTCATAATGCCAGTGTAA |
| | | ACTTGTCAGTGATTGGACTGAAGACCTTAGATCTCCTCCTAACTT |
| | | CAGGTAAAATCACCTTGCTGATATTGGATGAAGAAAGTGATATTT |
| | | TCATGTTAATTTTTGATGCCATGCACTCATTTCCAGCCAATGATG |
| | | AAGTCCAGAAACTTGGATGCAAAGCTTTACATGTGCTGTTTGAGA |
| | | GAGTCTCAGAGGAGCAACTGACTGAATTTGTTGAGAACAAAGAT |
| | | TATATGATATTGTTAAGTGCGTTAACAAATTTTAAAGATGAAGAG |
| | | GAAATTGTGCTTCATGTGCTGCATTGTTTACATTCCCTAGCGATTC |
| | | CTTGCAATAATGTGGAAGTCCTCATGAGTGGCAATGTCAGGTGTT |
| | | ATAATATTGTGGTGGAAGCTATGAAAGCATTCCCTATGAGTGAA |
| | | AGAATTCAAGAAGTGAGTTGCTGTTTGCTCCATAGGCTTACATTA |
| | | GGTAATTTTTTCAATATCCTGGTATTAAACGAAGTCCATGAGTTT |
| | | GTGGTGAAAGCTGTGCAGCAGTACCCAGAGAATGCAGCATTGCA |
| | | GATCTCAGCGCTCAGCTGTTTGGCCCTCCTCACTGAGACTATTTTC |
| | | TTAAATCAAGATTTAGAGGAAAAGAATGAGAATCAAGAGAATGA |
| | | TGATGAGGGGGAAGAAGATAAATTGTTTTGGCTGGAAGCCTGTT |
| | | ACAAAGCATTAACGTGGCATAGAAAGAACAAGCACGTGCAGGA |
| | | GGCCGCATGCTGGGCACTAAATAATCTCCTTATGTACCAAAACAG |
| | | TTTACATGAGAAGATTGGAGATGAAGATGGCCATTTCCCAGCTCA |
| | | TAGGGAAGTGATGCTCTCCATGCTGATGCATTCTTCATCAAAGGA |
| | | AGTTTTCCAGGCATCTGCGAATGCATTGTCAACTCTCTTAGAACA |
| | | AAATGTTAATTTCAGAAAAATACTGTTATCAAAAGGAATACACCT |
| | | GAATGTTTTGGAGTTAATGCAGAAGCATATACATTCTCCTGAAGT |
| | | GGCTGAAAGTGGCTGTAAAATGCTAAATCATCTTTTTGAAGGAA |
| | | GCAACACTTCCCTGGATATAATGGCAGCAGTGGTCCCCAAAATA |
| | | CTAACAGTTATGAAACGTCATGAGACATCATTACCAGTGCAGCTG |
| | | GAGGCGCTTCGAGCTATTTTACATTTTATAGTGCCTGGCATGCCA |
| | | GAAGAATCCAGGGAGGATACAGAATTTCATCATAAGCTAAATAT |
| | | GGTTAAAAAACAGTGTTTCAAGAATGATATTCACAAACTGGTCCT |
| | | AGCAGCTTTGAACAGGTTCATTGGAAATCCTGGGATTCAGAAAT |
| | | GTGGATTAAAAGTAATTTCTTCTATTGTACATTTTCCTGATGCATT |
| | | AGAGATGTTATCCCTGGAAGGTGCTATGGATTCAGTGCTTCACAC |
| | | ACTGCAGATGTATCCAGATGACCAAGAAATTCAGTGTCTGGGTTT |
| | | AAGTCTTATAGGATACTTGATTACAAAGAAGAATGTGTTCATAGG |
| | | AACTGGACATCTGCTGGCAAAAATTCTGGTTTCCAGCTTATACCG |
| | | ATTTAAGGATGTTGCTGAAATACAGACTAAAGGATTTCAGACAA |
| | | TCTTAGCAATCCTCAAATTGTCAGCATCTTTTTCTAAGCTGCTGGT |
| | | GCATCATTCATTTGACTTAGTAATATTCCATCAAATGTCTTCCAAT |
| | | ATCATGGAACAAAAGGATCAACAGTTTCTAAACCTCTGTTGCAA |
| | | GTGTTTTGCAAAAGTAGCTATGGATGATTACTTAAAAAATGTGAT |
| | | GCTAGAGAGAGCGTGTGATCAGAATAACAGCATCATGGTTGAAT |
| | | GCTTGCTTCTATTGGGAGCAGATGCCAATCAAGCAAAGGAGGGA |
| | | TCTTCTTTAATTTGTCAGGTATGTGAGAAAGAGAGCAGTCCCAAA |
| | | TTGGTGGAACTCTTACTGAATAGTGGATCTCGTGAACAAGATGTA |
| | | CGAAAAGCGTTGACGATAAGCATTGGGAAAGGTGACAGCCAGAT |
| | | CATCAGCTTGCTCTTAAGGAGGCTGGCCCTGGATGTGGCCAACAA |
| | | TAGCATTTGCCTTGGAGGATTTTGTATAGGAAAAGTTGAACCTTC |
| | | TTGGCTTGGTCCTTTATTTCCAGATAAGACTTCTAATTTAAGGAA |
| | | ACAAACAAATATAGCATCTACACTAGCAAGAATGGTGATCAGAT |
| | | ATCAGATGAAAAGTGCTGTGGAAGAAGGAACAGCCTCAGGCAGC |
| | | GATGGAAATTTTTCTGAAGATGTGCTGTCTAAATTTGATGAATGG |
| | | ACCTTTATTCCTGACTCTTCTATGGACAGTGTGTTTGCTCAAAGTG |
| | | ATGACCTGGATAGTGAAGGAAGTGAAGGCTCATTTCTTGTGAAA |
| | | AAGAAATCTAATTCAATTAGTGTAGGAGAATTTTACCGAGATGCC |
| | | GTATTACAGCGTTGCTCACCAAATTTGCAAAGACATTCCAATTCC |
| | | TTGGGGCCCATTTTTGATCATGAAGATTTACTGAAGCGAAAAAGA |
| | | AAAATATTATCTTCAGATGATTCACTCAGGTCATCAAAACTTCAA |
| | | TCCCATATGAGGCATTCAGACAGCATTTCTTCTCTGGCTTCTGAG |
| | | AGAGAATATATTACATCACTAGACCTTTCAGCAAATGAACTAAG |
| | | AGATATTGATGCCCTAAGCCAGAAATGCTGTATAAGTGTTCATTT |
| | | GGAGCATCTTGAAAAGCTGGAGCTTCACCAGAATGCACTCACGA |
| | | GCTTTCCACAACAGCTATGTGAAACTCTGAAGAGTTTGACACATT |
| | | TGGACTTGCACAGTAATAAATTTACATCATTTCCTTCTTATTTGTT |
| | | GAAAATGAGTTGTATTGCTAATCTTGATGTCTCTCGAAATGACAT |
| | | TGGACCCTCAGTGGTTTTAGATCCTACAGTGAAATGTCCAACTCT |
| | | GAAACAGTTTAACCTGTCATATAACCAGCTGTCTTTTGTACCTGA |
| | | GAACCTCACTGATGTGGTAGAGAAACTGGAGCAGCTCATTTTAG |
| | | AAGGAAATAAAATATCAGGGATATGCTCCCCCTTGAGACTGAAG |
| | | GAACTGAAGATTTTAAACCTTAGTAAGAACCACATTTCATCCCTA |
| | | TCAGAGAACTTTCTTGAGGCTTGTCCTAAAGTGGAGAGTTTCAGT |
| | | GCCAGAATGAATTTTCTTGCTGCTATGCCTTTCTTGCCTCCTTCTA |
| | | TGACAATCCTAAAATTATCTCAGAACAAATTTTCCTGTATTCCAG |
| | | AAGCAATTTTAAATCTTCCACACTTGCGGTCTTTAGATATGAGCA |
| | | GCAATGATATTCAGTACCTACCAGGTCCCGCACACTGGAAATCTT |
| | | TGAACTTAAGGGAACTCTTATTTAGCCATAATCAGATCAGCATCT |
| | | TGGACTTGAGTGAAAAAGCATATTTATGGTCTAGAGTAGAGAAA |
| | | CTGCATCTTTCTCACAATAAACTGAAAGAGATTCCTCCTGAGATT |
| | | GGCTGTCTTGAAAATCTGACATCTCTGGATGTCAGTTACAACTTG |
| | | GAACTAAGATCCTTTCCCAATGAAATGGGGAAATTAAGCAAAAT |
| | | ATGGGATCTTCCTTTGGATGAACTGCATCTTAACTTTGATTTTAAA |
| | | CATATAGGATGTAAAGCCAAAGACATCATAAGGTTTCTTCAACA |
| | | GCGATTAAAAAAGGCTGTGCCTTATAACCGAATGAAACTTATGA |
| | | TTGTGGGAAATACTGGGAGTGGTAAAACCACCTTATTGCAGCAA |
| | | TTAATGAAAACCAAGAAATCAGATCTTGGAATGCAAAGTGCCAC |
| | | AGTTGGCATAGATGTGAAAGACTGGCCTATCCAAATAAGAGACA |
| | | AAAGAAAGAGAGATCTCGTCCTAAATGTGTGGGATTTTGCAGGT |
| | | CGTGAGGAATTCTATAGTACTCATCCCCATTTTATGACGCAGCGA |
| | | GCATTGTACCTTGCTGTCTATGACCTCAGCAAGGGACAGGCTGAA |
| | | GTTGATGCCATGAAGCCTTGGCTCTTCAATATAAAGGCTCGCGCT |
| | | TCTTCTTCCCCTGTGATTCTCGTTGGCACACATTTGGATGTTTCTG |
| | | ATGAGAAGCAACGCAAAGCCTGCATGAGTAAAATCACCAAGGAA |
| | | CTCCTGAATAAGCGAGGGTTCCCTGCCATACGAGATTACCACTTT |
| | | GTGAATGCCACCGAGGAATCTGATGCTTTGGCAAAACTTCGGAA |
| | | AACCATCATAAACGAGAGCCTTAATTTCAAGATCCGAGATCAGC |
| | | TTGTTGTTGGACAGCTGATTCCAGACTGCTATGTAGAACTTGAAA |
| | | AAATCATTTTATCGGAGCGTAAAAATGTGCCAATTGAATTTCCCG |
| | | TAATTGACCGGAAACGATTATTACAACTAGTGAGAGAAAATCAG |
| | | CTGCAGTTAGATGAAAATGAGCTTCCTCACGCAGTTCACTTTCTA |
| | | AATGAATCAGGAGTCCTTCTTCATTTTCAAGACCCAGCACTGCAG |
| | | TTAAGTGACTTGTACTTTGTGGAACCCAAGTGGCTTTGTAAAATC |
| | | ATGGCACAGATTTTGACAGTGAAAGTGGAAGGTTGTCCAAAACA |
| | | CCCTAAGGGCATTATTTCGCGTAGAGATGTGGAAAAATTTCTTTC |
| | | AAAAAAAAGGAAATTTCCAAAGAACTACATGTCACAGTATTTTA |
| | | AGCTCCTAGAAAAATTCCAGATTGCTTTGCCAATAGGAGAAGAA |
| | | TATTTGCTGGTTCCAAGCAGTTTGTCTGACCACAGGCCTGTGATA |
| | | GAGCTTCCCCATTGTGAGAACTCTGAAATTATCATCCGACTATAT |
| | | GAAATGCCTTATTTTCCAATGGGATTTTGGTCAAGATTAATCAAT |
| | | CGATTACTTGAGATTTCACCTTACATGCTTTCAGGGAGAGAACGA |
| | | GCACTTCGCCCAAACAGAATGTATTGGCGACAAGGCATTTACTTA |
| | | AATTGGTCTCCTGAAGCTTATTGTCTGGTAGGATCTGAAGTCTTA |
| | | GACAATCATCCAGAGAGTTTCTTAAAAATTACAGTTCCTTCTTGT |
| | | AGAAAAGGCTGTATTCTTTTGGGCCAAGTTGTGGACCACATTGAT |
| | | TCTCTCATGGAAGAATGGTTTCCTGGGTTGCTGGAGATTGATATT |
| | | TGTGGTGAAGGAGAAACTCTGTTGAAGAAATGGGCATTATATAG |
| | | TTTTAATGATGGTGAAGAACATCAAAAAATCTTACTTGATGACTT |
| | | GATGAAGAAAGCAGAGGAAGGAGATCTCTTAGTAAATCCAGATC |
| | | AACCAAGGCTCACCATTCCAATATCTCAGATTGCCCCTGACTTGA |
| | | TTTTGGCTGACCTGCCTAGAAATATTATGTTGAATAATGATGAGT |
| | | TGGAATTTGAACAAGCTCCAGAGTTTCTCCTAGGTGATGGCAGTT |
| | | TTGGATCAGTTTACCGAGCAGCCTATGAAGGAGAAGAAGTGGCT |
| | | GTGAAGATTTTTAATAAACATACATCACTCAGGCTGTTAAGACAA |
| | | GAGCTTGTGGTGCTTTGCCACCTCCACCACCCCAGTTTGATATCTT |
| | | TGCTGGCAGCTGGGATTCGTCCCCGGATGTTGGTGATGGAGTTAG |
| | | CCTCCAAGGGTTCCTTGGATCGCCTGCTTCAGCAGGACAAAGCCA |
| | | GCCTCACTAGAACCCTACAGCACAGGATTGCACTCCACGTAGCTG |
| | | ATGGTTTGAGATACCTCCACTCAGCCATGATTATATACCGAGACC |
| | | TGAAACCCCACAATGTGCTGCTTTTCACACTGTATCCCAATGCTG |
| | | CCATCATTGCAAAGATTGCTGACTACGGCATTGCTCAGTACTGCT |
| | | GTAGAATGGGGATAAAAACATCAGAGGGCACACCAGGGTTTCGT |
| | | GCACCTGAAGTTGCCAGAGGAAATGTCATTTATAACCAACAGGC |
| | | TGATGTTTATTCATTTGGTTTACTACTCTATGACATTTTGACAACT |
| | | GGAGGTAGAATAGTAGAGGGTTTGAAGTTTCCAAATGAGTTTGA |
| | | TGAATTAGAAATACAAGGAAAATTACCTGATCCAGTTAAAGAAT |
| | | ATGGTTGTGCCCCATGGCCTATGGTTGAGAAATTAATTAAACAGT |
| | | GTTTGAAAGAAAATCCTCAAGAAAGGCCTACTTCTGCCCAGGTCT |
| | | TTGACATTTTGAATTCAGCTGAATTAGTCTGTCTGACGAGACGCA |
| | | TTTTATTACCTAAAAACGTAATTGTTGAATGCATGGTTGCTACAC |
| | | ATCACAACAGCAGGAATGCAAGCATTTGGCTGGGCTGTGGGCAC |
| | | ACCGACAGAGGACAGCTCTCATTTCTTGACTTAAATACTGAAGGA |
| | | TACACTTCTGAGGAAGTTGCTGATAGTAGAATATTGTGCTTAGCC |
| | | TTGGTGCATCTTCCTGTTGAAAAGGAAAGCTGGATTGTGTCTGGG |
| | | ACACAGTCTGGTACTCTCCTGGTCATCAATACCGAAGATGGGAA |
| | | AAAGAGACATACCCTAGAAAAGATGACTGATTCTGTCACTTGTTT |
| | | GTATTGCAATTCCTTTTCCAAGCAAAGCAAACAAAAAAATTTTCT |
| | | TTTGGTTGGAACCGCTGATGGCAAGTTAGCAATTTTTGAAGATAA |
| | | GACTGTTAAGCTTAAAGGAGCTGCTCCTTTGAAGATACTAAATAT |
| | | AGGAAATGTCAGTACTCCATTGATGTGTTTGAGTGAATCCACAAA |
| | | TTCAACGGAAAGAAATGTAATGTGGGGAGGATGTGGCACAAAGA |
| | | TTTTCTCCTTTTCTAATGATTTCACCATTCAGAAACTCATTGAGAC |
| | | AAGAACAAGCCAACTGTTTTCTTATGCAGCTTTCAGTGATTCCAA |
| | | CATCATAACAGTGGTGGTAGACACTGCTCTCTATATTGCTAAGCA |
| | | AAATAGCCCTGTTGTGGAAGTGTGGGATAAGAAAACTGAAAAAC |
| | | TCTGTGGACTAATAGACTGCGTGCACTTTTTAAGGGAGGTAATGG |
| | | TAAAAGAAAACAAGGAATCAAAACACAAAATGTCTTATTCTGGG |
| | | AGAGTGAAAACCCTCTGCCTTCAGAAGAACACTGCTCTTTGGATA |
| | | GGAACTGGAGGAGGCCATATTTTACTCCTGGATCTTTCAACTCGT |
| | | CGACTTATACGTGTAATTTACAACTTTTGTAATTCGGTCAGAGTC |
| | | ATGATGACAGCACAGCTAGAGATACAATCTTGCTTGACCGTTTGG |
| | | GACATCAATCTTCCACATGAAGTGCAAAATTTAGAAAAACACAT |
| | | TGAAGTGAGAAAAGAATTAGCTGAAAAAATGAGACGAACATCTG |
| | | TTGAGTAAGAGAGAAATAGGAATTGTCTTTGGATAGGAAAATTA |
| | | TTCTCTCCTCTTGTAAATATTTATTTTAAAAATGTTCACATGGAAA |
| | | GGGTACTCACATTTTTTGAAATAGCTCGTGTGTATGAAGGAATGT |
| | | TATTATTTTTAATTTAAATATATGTAAAAATACTTACCAGTAAAT |
| | | GTGTATTTTAAAGAACTATTTAAAACACAATGTTATATTTCTTAT |
| | | AAATACCAGTTACTTTCGTTCATTAATTAATGAAAATAAATCTGT |
| | | GAAGTACCTAATTTAAGTACTCATACTAAAATTTATAAGGCCGAT |
| | | AATTTTTTGTTTTCTTGTCTGTAATGGAGGTAAACTTTATTTTAAA |
| | | TTCTGTGCTTAAGACAGGACTATTGCTTGTCGATTTTTCTAGAAAT |
| | | CTGCACGGTATAATGAAAATATTAAGACAGTTTCCCATGTAATGT |
| | | ATTCCTTCTTAGATTGCATCGAAATGCACTATCATATATGCTTGTA |
| | | AATATTCAAATGAATTTGCACTAATAAAGTCCTTTGTTGGTATGT |
| | | GAATTCTCTTTGTTGCTGTTGCAAACAGTGCATCTTACACAACTTC |
| | | ACTCAATTCAAAAGAAAACTCCATTAAAAGTACTAATGAAAAAA |
| | | CATGACATACTGTCAAAGTCCTCATATCTAGGAAAGACACAGAA |
| | | ACTCTCTTTGTCACAGAAACTCTCTGTGTCTTTCCTAGACATAATA |
| | | GAGTTGTTTTTCAACTCTATGTTTGAATGTGGATACCCTGAATTTT |
| | | GTATAATTAGTGTAAATACAGTGTTCAGTCCTTCAAGTGATATTT |
| | | TTATTTTTTTATTCATACCACTAGCTACTTGTTTTCTAATCTGCTTC |
| | | ATTCTAATGCTTATATTCATCTTTTCCCTAAATTTGTGATGCTGCA |
| | | GATCCTACATCATTCAGATAGAAACCTTTTTTTTTTTCAGAATTAT |
| | | AGAATTCCACAGCTCCTACCAAGACCATGAGGATAAATATCTAA |
| | | CACTTTTCAGTTGCTGAAGGAGAAAGGAGCTTTAGTTATGATGGA |
| | | TAAAAATATCTGCCACCCTAGGCTTCCAAATTATACTTAAATTGT |
| | | TTACATAGCTTACCACAATAGGAGTATCAGGGCCAAATACCTATG |
| | | TAATAATTTGAGGTCATTTCTGCTTTAGGAAAAGTACTTTCGGTA |
| | | AATTCTTTGGCCCTGACCAGTATTCATTATTTCAGATAATTCCCTG |
| | | TGATAGGACAACTAGTACATTTAATATTCTCAGAACTTATGGCAT |
| | | TTTACTATGTGAAAACTTTAAATTTATTTATATTAAGGGTAATCA |
| | | AATTCTTAAAGATGAAAGATTTTCTGTATTTTAAAGGAAGCTATG |
| | | CTTTAACTTGTTATGTAATTAACAAAAAAATCATATATAATAGAG |
| | | CTCTTTGTTCCAGTGTTATCTCTTTCATTGTTACTTTGTATTTGCAA |
| | | TTTTTTTTACCAAAGACAAATTAAAAAAATGAATACCATATTTAA |
| | | ATGGAATAATAAAGGTTTTTTAAA |
| 8 | X3 | TTCAAACATCATAAGACCGGCACTCTCTCCCAAAGATACAAGCTG |
| | | TAGCAAGGAGTTTTGTGCATATCAGTTTCCCAGCTCATAGGGAAG |
| | | TGATGCTCTCCATGCTGATGCATTCTTCATCAAAGGAAGTTTTCC |
| | | AGGCATCTGCGAATGCATTGTCAACTCTCTTAGAACAAAATGTTA |
| | | ATTTCAGAAAAATACTGTTATCAAAAGGAATACACCTGAATGTTT |
| | | TGGAGTTAATGCAGAAGCATATACATTCTCCTGAAGTGGCTGAA |
| | | AGTGGCTGTAAAATGCTAAATCATCTTTTTGAAGGAAGCAACACT |
| | | TCCCTGGATATAATGGCAGCAGTGGTCCCCAAAATACTAACAGTT |
| | | ATGAAACGTCATGAGACATCATTACCAGTGCAGCTGGAGGCGCT |
| | | TCGAGCTATTTTACATTTTATAGTGCCTGGCATGCCAGAAGAATC |
| | | CAGGGAGGATACAGAATTTCATCATAAGCTAAATATGGTTAAAA |
| | | AACAGTGTTTCAAGAATGATATTCACAAACTGGTCCTAGCAGCTT |
| | | TGAACAGGTTCATTGGAAATCCTGGGATTCAGAAATGTGGATTA |
| | | AAAGTAATTTCTTCTATTGTACATTTTCCTGATGCATTAGAGATGT |
| | | TATCCCTGGAAGGTGCTATGGATTCAGTGCTTCACACACTGCAGA |
| | | TGTATCCAGATGACCAAGAAATTCAGTGTCTGGGTTTAAGTCTTA |
| | | TAGGATACTTGATTACAAAGAAGAATGTGTTCATAGGAACTGGA |
| | | CATCTGCTGGCAAAAATTCTGGTTTCCAGCTTATACCGATTTAAG |
| | | GATGTTGCTGAAATACAGACTAAAGGATTTCAGACAATCTTAGC |
| | | AATCCTCAAATTGTCAGCATCTTTTTCTAAGCTGCTGGTGCATCAT |
| | | TCATTTGACTTAGTAATATTCCATCAAATGTCTTCCAATATCATGG |
| | | AACAAAAGGATCAACAGTTTCTAAACCTCTGTTGCAAGTGTTTTG |
| | | CAAAAGTAGCTATGGATGATTACTTAAAAAATGTGATGCTAGAG |
| | | AGAGCGTGTGATCAGAATAACAGCATCATGGTTGAATGCTTGCTT |
| | | CTATTGGGAGCAGATGCCAATCAAGCAAAGGAGGGATCTTCTTT |
| | | AATTTGTCAGGTATGTGAGAAAGAGAGCAGTCCCAAATTGGTGG |
| | | AACTCTTACTGAATAGTGGATCTCGTGAACAAGATGTACGAAAA |
| | | GCGTTGACGATAAGCATTGGGAAAGGTGACAGCCAGATCATCAG |
| | | CTTGCTCTTAAGGAGGCTGGCCCTGGATGTGGCCAACAATAGCAT |
| | | TTGCCTTGGAGGATTTTGTATAGGAAAAGTTGAACCTTCTTGGCT |
| | | TGGTCCTTTATTTCCAGATAAGACTTCTAATTTAAGGAAACAAAC |
| | | AAATATAGCATCTACACTAGCAAGAATGGTGATCAGATATCAGA |
| | | TGAAAAGTGCTGTGGAAGAAGGAACAGCCTCAGGCAGCGATGGA |
| | | AATTTTTCTGAAGATGTGCTGTCTAAATTTGATGAATGGACCTTT |
| | | ATTCCTGACTCTTCTATGGACAGTGTGTTTGCTCAAAGTGATGAC |
| | | CTGGATAGTGAAGGAAGTGAAGGCTCATTTCTTGTGAAAAAGAA |
| | | ATCTAATTCAATTAGTGTAGGAGAATTTTACCGAGATGCCGTATT |
| | | ACAGCGTTGCTCACCAAATTTGCAAAGACATTCCAATTCCTTGGG |
| | | GCCCATTTTTGATCATGAAGATTTACTGAAGCGAAAAAGAAAAA |
| | | TATTATCTTCAGATGATTCACTCAGGTCATCAAAACTTCAATCCC |
| | | ATATGAGGCATTCAGACAGCATTTCTTCTCTGGCTTCTGAGAGAG |
| | | AATATATTACATCACTAGACCTTTCAGCAAATGAACTAAGAGATA |
| | | TTGATGCCCTAAGCCAGAAATGCTGTATAAGTGTTCATTTGGAGC |
| | | ATCTTGAAAAGCTGGAGCTTCACCAGAATGCACTCACGAGCTTTC |
| | | CACAACAGCTATGTGAAACTCTGAAGAGTTTGACACATTTGGACT |
| | | TGCACAGTAATAAATTTACATCATTTCCTTCTTATTTGTTGAAAAT |
| | | GAGTTGTATTGCTAATCTTGATGTCTCTCGAAATGACATTGGACC |
| | | CTCAGTGGTTTTAGATCCTACAGTGAAATGTCCAACTCTGAAACA |
| | | GTTTAACCTGTCATATAACCAGCTGTCTTTTGTACCTGAGAACCT |
| | | CACTGATGTGGTAGAGAAACTGGAGCAGCTCATTTTAGAAGGAA |
| | | ATAAAATATCAGGGATATGCTCCCCCTTGAGACTGAAGGAACTG |
| | | AAGATTTTAAACCTTAGTAAGAACCACATTTCATCCCTATCAGAG |
| | | AACTTTCTTGAGGCTTGTCCTAAAGTGGAGAGTTTCAGTGCCAGA |
| | | ATGAATTTTCTTGCTGCTATGCCTTTCTTGCCTCCTTCTATGACAA |
| | | TCCTAAAATTATCTCAGAACAAATTTTCCTGTATTCCAGAAGCAA |
| | | TTTTAAATCTTCCACACTTGCGGTCTTTAGATATGAGCAGCAATG |
| | | ATATTCAGTACCTACCAGGTCCCGCACACTGGAAATCTTTGAACT |
| | | TAAGGGAACTCTTATTTAGCCATAATCAGATCAGCATCTTGGACT |
| | | TGAGTGAAAAAGCATATTTATGGTCTAGAGTAGAGAAACTGCAT |
| | | CTTTCTCACAATAAACTGAAAGAGATTCCTCCTGAGATTGGCTGT |
| | | CTTGAAAATCTGACATCTCTGGATGTCAGTTACAACTTGGAACTA |
| | | AGATCCTTTCCCAATGAAATGGGGAAATTAAGCAAAATATGGGA |
| | | TCTTCCTTTGGATGAACTGCATCTTAACTTTGATTTTAAACATATA |
| | | GGATGTAAAGCCAAAGACATCATAAGGTTTCTTCAACAGCGATT |
| | | AAAAAAGGCTGTGCCTTATAACCGAATGAAACTTATGATTGTGG |
| | | GAAATACTGGGAGTGGTAAAACCACCTTATTGCAGCAATTAATG |
| | | AAAACCAAGAAATCAGATCTTGGAATGCAAAGTGCCACAGTTGG |
| | | CATAGATGTGAAAGACTGGCCTATCCAAATAAGAGACAAAAGAA |
| | | AGAGAGATCTCGTCCTAAATGTGTGGGATTTTGCAGGTCGTGAGG |
| | | AATTCTATAGTACTCATCCCCATTTTATGACGCAGCGAGCATTGT |
| | | ACCTTGCTGTCTATGACCTCAGCAAGGGACAGGCTGAAGTTGATG |
| | | CCATGAAGCCTTGGCTCTTCAATATAAAGGCTCGCGCTTCTTCTT |
| | | CCCCTGTGATTCTCGTTGGCACACATTTGGATGTTTCTGATGAGA |
| | | AGCAACGCAAAGCCTGCATGAGTAAAATCACCAAGGAACTCCTG |
| | | AATAAGCGAGGGTTCCCTGCCATACGAGATTACCACTTTGTGAAT |
| | | GCCACCGAGGAATCTGATGCTTTGGCAAAACTTCGGAAAACCAT |
| | | CATAAACGAGAGCCTTAATTTCAAGATCCGAGATCAGCTTGTTGT |
| | | TGGACAGCTGATTCCAGACTGCTATGTAGAACTTGAAAAAATCAT |
| | | TTTATCGGAGCGTAAAAATGTGCCAATTGAATTTCCCGTAATTGA |
| | | CCGGAAACGATTATTACAACTAGTGAGAGAAAATCAGCTGCAGT |
| | | TAGATGAAAATGAGCTTCCTCACGCAGTTCACTTTCTAAATGAAT |
| | | CAGGAGTCCTTCTTCATTTTCAAGACCCAGCACTGCAGTTAAGTG |
| | | ACTTGTACTTTGTGGAACCCAAGTGGCTTTGTAAAATCATGGCAC |
| | | AGATTTTGACAGTGAAAGTGGAAGGTTGTCCAAAACACCCTAAG |
| | | GGCATTATTTCGCGTAGAGATGTGGAAAAATTTCTTTCAAAAAAA |
| | | AGGAAATTTCCAAAGAACTACATGTCACAGTATTTTAAGCTCCTA |
| | | GAAAAATTCCAGATTGCTTTGCCAATAGGAGAAGAATATTTGCTG |
| | | GTTCCAAGCAGTTTGTCTGACCACAGGCCTGTGATAGAGCTTCCC |
| | | CATTGTGAGAACTCTGAAATTATCATCCGACTATATGAAATGCCT |
| | | TATTTTCCAATGGGATTTTGGTCAAGATTAATCAATCGATTACTT |
| | | GAGATTTCACCTTACATGCTTTCAGGGAGAGAACGAGCACTTCGC |
| | | CCAAACAGAATGTATTGGCGACAAGGCATTTACTTAAATTGGTCT |
| | | CCTGAAGCTTATTGTCTGGTAGGATCTGAAGTCTTAGACAATCAT |
| | | CCAGAGAGTTTCTTAAAAATTACAGTTCCTTCTTGTAGAAAAGGC |
| | | TGTATTCTTTTGGGCCAAGTTGTGGACCACATTGATTCTCTCATGG |
| | | AAGAATGGTTTCCTGGGTTGCTGGAGATTGATATTTGTGGTGAAG |
| | | GAGAAACTCTGTTGAAGAAATGGGCATTATATAGTTTTAATGATG |
| | | GTGAAGAACATCAAAAAATCTTACTTGATGACTTGATGAAGAAA |
| | | GCAGAGGAAGGAGATCTCTTAGTAAATCCAGATCAACCAAGGCT |
| | | CACCATTCCAATATCTCAGATTGCCCCTGACTTGATTTTGGCTGA |
| | | CCTGCCTAGAAATATTATGTTGAATAATGATGAGTTGGAATTTGA |
| | | ACAAGCTCCAGAGTTTCTCCTAGGTGATGGCAGTTTTGGATCAGT |
| | | TTACCGAGCAGCCTATGAAGGAGAAGAAGTGGCTGTGAAGATTT |
| | | TTAATAAACATACATCACTCAGGCTGTTAAGACAAGAGCTTGTGG |
| | | TGCTTTGCCACCTCCACCACCCCAGTTTGATATCTTTGCTGGCAGC |
| | | TGGGATTCGTCCCCGGATGTTGGTGATGGAGTTAGCCTCCAAGGG |
| | | TTCCTTGGATCGCCTGCTTCAGCAGGACAAAGCCAGCCTCACTAG |
| | | AACCCTACAGCACAGGATTGCACTCCACGTAGCTGATGGTTTGAG |
| | | ATACCTCCACTCAGCCATGATTATATACCGAGACCTGAAACCCCA |
| | | CAATGTGCTGCTTTTCACACTGTATCCCAATGCTGCCATCATTGC |
| | | AAAGATTGCTGACTACGGCATTGCTCAGTACTGCTGTAGAATGGG |
| | | GATAAAAACATCAGAGGGCACACCAGGGTTTCGTGCACCTGAAG |
| | | TTGCCAGAGGAAATGTCATTTATAACCAACAGGCTGATGTTTATT |
| | | CATTTGGTTTACTACTCTATGACATTTTGACAACTGGAGGTAGAA |
| | | TAGTAGAGGGTTTGAAGTTTCCAAATGAGTTTGATGAATTAGAAA |
| | | TACAAGGAAAATTACCTGATCCAGTTAAAGAATATGGTTGTGCCC |
| | | CATGGCCTATGGTTGAGAAATTAATTAAACAGTGTTTGAAAGAA |
| | | AATCCTCAAGAAAGGCCTACTTCTGCCCAGGTCTTTGACATTTTG |
| | | AATTCAGCTGAATTAGTCTGTCTGACGAGACGCATTTTATTACCT |
| | | AAAAACGTAATTGTTGAATGCATGGTTGCTACACATCACAACAG |
| | | CAGGAATGCAAGCATTTGGCTGGGCTGTGGGCACACCGACAGAG |
| | | GACAGCTCTCATTTCTTGACTTAAATACTGAAGGATACACTTCTG |
| | | AGGAAGTTGCTGATAGTAGAATATTGTGCTTAGCCTTGGTGCATC |
| | | TTCCTGTTGAAAAGGAAAGCTGGATTGTGTCTGGGACACAGTCTG |
| | | GTACTCTCCTGGTCATCAATACCGAAGATGGGAAAAAGAGACAT |
| | | ACCCTAGAAAAGATGACTGATTCTGTCACTTGTTTGTATTGCAAT |
| | | TCCTTTTCCAAGCAAAGCAAACAAAAAAATTTTCTTTTGGTTGGA |
| | | ACCGCTGATGGCAAGTTAGCAATTTTTGAAGATAAGACTGTTAAG |
| | | CTTAAAGGAGCTGCTCCTTTGAAGATACTAAATATAGGAAATGTC |
| | | AGTACTCCATTGATGTGTTTGAGTGAATCCACAAATTCAACGGAA |
| | | AGAAATGTAATGTGGGGAGGATGTGGCACAAAGATTTTCTCCTTT |
| | | TCTAATGATTTCACCATTCAGAAACTCATTGAGACAAGAACAAGC |
| | | CAACTGTTTTCTTATGCAGCTTTCAGTGATTCCAACATCATAACA |
| | | GTGGTGGTAGACACTGCTCTCTATATTGCTAAGCAAAATAGCCCT |
| | | GTTGTGGAAGTGTGGGATAAGAAAACTGAAAAACTCTGTGGACT |
| | | AATAGACTGCGTGCACTTTTTAAGGGAGGTAATGGTAAAAGAAA |
| | | ACAAGGAATCAAAACACAAAATGTCTTATTCTGGGAGAGTGAAA |
| | | ACCCTCTGCCTTCAGAAGAACACTGCTCTTTGGATAGGAACTGGA |
| | | GGAGGCCATATTTTACTCCTGGATCTTTCAACTCGTCGACTTATA |
| | | CGTGTAATTTACAACTTTTGTAATTCGGTCAGAGTCATGATGACA |
| | | GCACAGCTAGGAAGCCTTAAAAATGTCATGCTGGTATTGGGCTA |
| | | CAACCGGAAAAATACTGAAGGTACACAAAAGCAGAAAGAGATA |
| | | CAATCTTGCTTGACCGTTTGGGACATCAATCTTCCACATGAAGTG |
| | | CAAAATTTAGAAAAACACATTGAAGTGAGAAAAGAATTAGCTGA |
| | | AAAAATGAGACGAACATCTGTTGAGTAAGAGAGAAATAGGAATT |
| | | GTCTTTGGATAGGAAAATTATTCTCTCCTCTTGTAAATATTTATTT |
| | | TAAAAATGTTCACATGGAAAGGGTACTCACATTTTTTGAAATAGC |
| | | TCGTGTGTATGAAGGAATGTTATTATTTTTAATTTAAATATATGTA |
| | | AAAATACTTACCAGTAAATGTGTATTTTAAAGAACTATTTAAAAC |
| | | ACAATGTTATATTTCTTATAAATACCAGTTACTTTCGTTCATTAAT |
| | | TAATGAAAATAAATCTGTGAAGTACCTAATTTAAGTACTCATACT |
| | | AAAATTTATAAGGCCGATAATTTTTTGTTTTCTTGTCTGTAATGGA |
| | | GGTAAACTTTATTTTAAATTCTGTGCTTAAGACAGGACTATTGCT |
| | | TGTCGATTTTTCTAGAAATCTGCACGGTATAATGAAAATATTAAG |
| | | ACAGTTTCCCATGTAATGTATTCCTTCTTAGATTGCATCGAAATG |
| | | CACTATCATATATGCTTGTAAATATTCAAATGAATTTGCACTAAT |
| | | AAAGTCCTTTGTTGGTATGTGAATTCTCTTTGTTGCTGTTGCAAAC |
| | | AGTGCATCTTACACAACTTCACTCAATTCAAAAGAAAACTCCATT |
| | | AAAAGTACTAATGAAAAAACATGACATACTGTCAAAGTCCTCAT |
| | | ATCTAGGAAAGACACAGAAACTCTCTTTGTCACAGAAACTCTCTG |
| | | TGTCTTTCCTAGACATAATAGAGTTGTTTTTCAACTCTATGTTTGA |
| | | ATGTGGATACCCTGAATTTTGTATAATTAGTGTAAATACAGTGTT |
| | | CAGTCCTTCAAGTGATATTTTTATTTTTTTATTCATACCACTAGCT |
| | | ACTTGTTTTCTAATCTGCTTCATTCTAATGCTTATATTCATCTTTTC |
| | | CCTAAATTTGTGATGCTGCAGATCCTACATCATTCAGATAGAAAC |
| | | CTTTTTTTTTTTCAGAATTATAGAATTCCACAGCTCCTACCAAGAC |
| | | CATGAGGATAAATATCTAACACTTTTCAGTTGCTGAAGGAGAAA |
| | | GGAGCTTTAGTTATGATGGATAAAAATATCTGCCACCCTAGGCTT |
| | | CCAAATTATACTTAAATTGTTTACATAGCTTACCACAATAGGAGT |
| | | ATCAGGGCCAAATACCTATGTAATAATTTGAGGTCATTTCTGCTT |
| | | TAGGAAAAGTACTTTCGGTAAATTCTTTGGCCCTGACCAGTATTC |
| | | ATTATTTCAGATAATTCCCTGTGATAGGACAACTAGTACATTTAA |
| | | TATTCTCAGAACTTATGGCATTTTACTATGTGAAAACTTTAAATTT |
| | | ATTTATATTAAGGGTAATCAAATTCTTAAAGATGAAAGATTTTCT |
| | | GTATTTTAAAGGAAGCTATGCTTTAACTTGTTATGTAATTAACAA |
| | | AAAAATCATATATAATAGAGCTCTTTGTTCCAGTGTTATCTCTTTC |
| | | ATTGTTACTTTGTATTTGCAATTTTTTTTACCAAAGACAAATTAAA |
| | | AAAATGAATACCATATTTAAATGGAATAATAAAGGTTTTTTAAAA |
| | | ACTT |
| 9 | X4 | CCTGAGTGGGGGAGGAGGAAGCCGAGCAGGAGGGCTCCGGAGA |
| | | GGGAGGGCAACGCGGGGCGGGGAGCTGCCTCCTTCCTCATAAAC |
| | | AGGCGGGCGTGGGCGCCGATGGGGCCCGCGGGGAGCGCTGGCTG |
| | | CGGGCGGTGAGCTGAGCTCGCCCCCGGGGAGCTGTGGCCGGCGC |
| | | CCCTGCCGGTTCCCTGAGCAGCGGACGTTCATGCTGGGAGGGCG |
| | | GCGGGTTGGAAGCAGGTGCCACCATGGCTAGTGGCAGCTGTCAG |
| | | GGGTGCGAAGAGGACGAGGAAACTCTGAAGAAGTTGATAGTCAG |
| | | GCTGAACAATGTCCAGGAAGGAAAACAGATAGAAACGCTGGTCC |
| | | AAATCCTGGAGGATCTGCTGGTGTTCACGTACTCCGAGCGCGCCT |
| | | CCAAGTTATTTCAAGGCAAAAATATCCATGTGCCTCTGTTGATCG |
| | | TCTTGGACTCCTATATGAGAGTCGCGAGTGTGCAGCAGGTGGGTT |
| | | GGTCACTTCTGTGCAAATTAATAGAAGTCTGTCCAGGTACAATGC |
| | | AAAGCTTAATGGGACCCCAGGATGTTGGAAATGATTGGGAAGTC |
| | | CTTGGTGTTCACCAATTGATTCTTAAAATGCTAACAGTTCATAAT |
| | | GCCAGTGTAAACTTGTCAGTGATTGGACTGAAGACCTTAGATCTC |
| | | CTCCTAACTTCAGGTAAAATCACCTTGCTGATATTGGATGAAGAA |
| | | AGTGATATTTTCATGTTAATTTTTGATGCCATGCACTCATTTCCAG |
| | | CCAATGATGAAGTCCAGAAACTTGGATGCAAAGCTTTACATGTG |
| | | CTGTTTGAGAGAGTCTCAGAGGAGCAACTGACTGAATTTGTTGAG |
| | | AACAAAGATTATATGATATTGTTAAGTGCGTTAACAAATTTTAAA |
| | | GATGAAGAGGAAATTGTGCTTCATGTGCTGCATTGTTTACATTCC |
| | | CTAGCGATTCCTTGCAATAATGTGGAAGTCCTCATGAGTGGCAAT |
| | | GTCAGGTGTTATAATATTGTGGTGGAAGCTATGAAAGCATTCCCT |
| | | ATGAGTGAAAGAATTCAAGAAGTGAGTTGCTGTTTGCTCCATAG |
| | | GCTTACATTAGGTAATTTTTTCAATATCCTGGTATTAAACGAAGT |
| | | CCATGAGTTTGTGGTGAAAGCTGTGCAGCAGTACCCAGAGAATG |
| | | CAGCATTGCAGATCTCAGCGCTCAGCTGTTTGGCCCTCCTCACTG |
| | | AGACTATTTTCTTAAATCAAGATTTAGAGGAAAAGAATGAGAAT |
| | | CAAGAGAATGATGATGAGGGGGAAGAAGATAAATTGTTTTGGCT |
| | | GGAAGCCTGTTACAAAGCATTAACGTGGCATAGAAAGAACAAGC |
| | | ACGTGCAGGAGGCCGCATGCTGGGCACTAAATAATCTCCTTATGT |
| | | ACCAAAACAGTTTACATGAGAAGATTGGAGATGAAGATGGCCAT |
| | | TTCCCAGCTCATAGGGAAGTGATGCTCTCCATGCTGATGCATTCT |
| | | TCATCAAAGGAAGTTTTCCAGGCATCTGCGAATGCATTGTCAACT |
| | | CTCTTAGAACAAAATGTTAATTTCAGAAAAATACTGTTATCAAAA |
| | | GGAATACACCTGAATGTTTTGGAGTTAATGCAGAAGCATATACAT |
| | | TCTCCTGAAGTGGCTGAAAGTGGCTGTAAAATGCTAAATCATCTT |
| | | TTTGAAGGAAGCAACACTTCCCTGGATATAATGGCAGCAGTGGT |
| | | CCCCAAAATACTAACAGTTATGAAACGTCATGAGACATCATTACC |
| | | AGTGCAGCTGGAGGCGCTTCGAGCTATTTTACATTTTATAGTGCC |
| | | TGGCATGCCAGAAGAATCCAGGGAGGATACAGAATTTCATCATA |
| | | AGCTAAATATGGTTAAAAAACAGTGTTTCAAGAATGATATTCAC |
| | | AAACTGGTCCTAGCAGCTTTGAACAGGTTCATTGGAAATCCTGGG |
| | | ATTCAGAAATGTGGATTAAAAGTAATTTCTTCTATTGTACATTTTC |
| | | CTGATGCATTAGAGATGTTATCCCTGGAAGGTGCTATGGATTCAG |
| | | TGCTTCACACACTGCAGATGTATCCAGATGACCAAGAAATTCAGT |
| | | GTCTGGGTTTAAGTCTTATAGGATACTTGATTACAAAGAAGAATG |
| | | TGTTCATAGGAACTGGACATCTGCTGGCAAAAATTCTGGTTTCCA |
| | | GCTTATACCGATTTAAGGATGTTGCTGAAATACAGACTAAAGGAT |
| | | TTCAGACAATCTTAGCAATCCTCAAATTGTCAGCATCTTTTTCTAA |
| | | GCTGCTGGTGCATCATTCATTTGACTTAGTAATATTCCATCAAAT |
| | | GTCTTCCAATATCATGGAACAAAAGGATCAACAGTTTCTAAACCT |
| | | CTGTTGCAAGTGTTTTGCAAAAGTAGCTATGGATGATTACTTAAA |
| | | AAATGTGATGCTAGAGAGAGCGTGTGATCAGAATAACAGCATCA |
| | | TGGTTGAATGCTTGCTTCTATTGGGAGCAGATGCCAATCAAGCAA |
| | | AGGAGGGATCTTCTTTAATTTGTCAGGTATGTGAGAAAGAGAGC |
| | | AGTCCCAAATTGGTGGAACTCTTACTGAATAGTGGATCTCGTGAA |
| | | CAAGATGTACGAAAAGCGTTGACGATAAGCATTGGGAAAGGTGA |
| | | CAGCCAGATCATCAGCTTGCTCTTAAGGAGGCTGGCCCTGGATGT |
| | | GGCCAACAATAGCATTTGCCTTGGAGGATTTTGTATAGGAAAAGT |
| | | TGAACCTTCTTGGCTTGGTCCTTTATTTCCAGATAAGACTTCTAAT |
| | | TTAAGGAAACAAACAAATATAGCATCTACACTAGCAAGAATGGT |
| | | GATCAGATATCAGATGAAAAGTGCTGTGGAAGAAGGAACAGCCT |
| | | CAGGCAGCGATGGAAATTTTTCTGAAGATGTGCTGTCTAAATTTG |
| | | ATGAATGGACCTTTATTCCTGACTCTTCTATGGACAGTGTGTTTGC |
| | | TCAAAGTGATGACCTGGATAGTGAAGGAAGTGAAGGCTCATTTC |
| | | TTGTGAAAAAGAAATCTAATTCAATTAGTGTAGGAGAATTTTACC |
| | | GAGATGCCGTATTACAGCGTTGCTCACCAAATTTGCAAAGACATT |
| | | CCAATTCCTTGGGGCCCATTTTTGATCATGAAGATTTACTGAAGC |
| | | GAAAAAGAAAAATATTATCTTCAGATGATTCACTCAGGTCATCA |
| | | AAACTTCAATCCCATATGAGGCATTCAGACAGCATTTCTTCTCTG |
| | | GCTTCTGAGAGAGAATATATTACATCACTAGACCTTTCAGCAAAT |
| | | GAACTAAGAGATATTGATGCCCTAAGCCAGAAATGCTGTATAAG |
| | | TGTTCATTTGGAGCATCTTGAAAAGCTGGAGCTTCACCAGAATGC |
| | | ACTCACGAGCTTTCCACAACAGCTATGTGAAACTCTGAAGAGTTT |
| | | GACACATTTGGACTTGCACAGTAATAAATTTACATCATTTCCTTC |
| | | TTATTTGTTGAAAATGAGTTGTATTGCTAATCTTGATGTCTCTCGA |
| | | AATGACATTGGACCCTCAGTGGTTTTAGATCCTACAGTGAAATGT |
| | | CCAACTCTGAAACAGTTTAACCTGTCATATAACCAGCTGTCTTTT |
| | | GTACCTGAGAACCTCACTGATGTGGTAGAGAAACTGGAGCAGCT |
| | | CATTTTAGAAGGAAATAAAATATCAGGGATATGCTCCCCCTTGAG |
| | | ACTGAAGGAACTGAAGATTTTAAACCTTAGTAAGAACCACATTTC |
| | | ATCCCTATCAGAGAACTTTCTTGAGGCTTGTCCTAAAGTGGAGAG |
| | | TTTCAGTGCCAGAATGAATTTTCTTGCTGCTATGCCTTTCTTGCCT |
| | | CCTTCTATGACAATCCTAAAATTATCTCAGAACAAATTTTCCTGT |
| | | ATTCCAGAAGCAATTTTAAATCTTCCACACTTGCGGTCTTTAGAT |
| | | ATGAGCAGCAATGATATTCAGTACCTACCAGGTCCCGCACACTG |
| | | GAAATCTTTGAACTTAAGGGAACTCTTATTTAGCCATAATCAGAT |
| | | CAGCATCTTGGACTTGAGTGAAAAAGCATATTTATGGTCTAGAGT |
| | | AGAGAAACTGCATCTTTCTCACAATAAACTGAAAGAGATTCCTCC |
| | | TGAGATTGGCTGTCTTGAAAATCTGACATCTCTGGATGTCAGTTA |
| | | CAACTTGGAACTAAGATCCTTTCCCAATGAAATGGGGAAATTAA |
| | | GCAAAATATGGGATCTTCCTTTGGATGAACTGCATCTTAACTTTG |
| | | ATTTTAAACATATAGGATGTAAAGCCAAAGACATCATAAGGTTTC |
| | | TTCAACAGCGATTAAAAAAGGCTGTGCCTTATAACCGAATGAAA |
| | | CTTATGATTGTGGGAAATACTGGGAGTGGTAAAACCACCTTATTG |
| | | CAGCAATTAATGAAAACCAAGAAATCAGATCTTGGAATGCAAAG |
| | | TGCCACAGTTGGCATAGATGTGAAAGACTGGCCTATCCAAATAA |
| | | GAGACAAAAGAAAGAGAGATCTCGTCCTAAATGTGTGGGATTTT |
| | | GCAGGTCGTGAGGAATTCTATAGTACTCATCCCCATTTTATGACG |
| | | CAGCGAGCATTGTACCTTGCTGTCTATGACCTCAGCAAGGGACAG |
| | | GCTGAAGTTGATGCCATGAAGCCTTGGCTCTTCAATATAAAGGCT |
| | | CGCGCTTCTTCTTCCCCTGTGATTCTCGTTGGCACACATTTGGATG |
| | | TTTCTGATGAGAAGCAACGCAAAGCCTGCATGAGTAAAATCACC |
| | | AAGGAACTCCTGAATAAGCGAGGGTTCCCTGCCATACGAGATTA |
| | | CCACTTTGTGAATGCCACCGAGGAATCTGATGCTTTGGCAAAACT |
| | | TCGGAAAACCATCATAAACGAGAGCCTTAATTTCAAGATCCGAG |
| | | ATCAGCTTGTTGTTGGACAGCTGATTCCAGACTGCTATGTAGAAC |
| | | TTGAAAAAATCATTTTATCGGAGCGTAAAAATGTGCCAATTGAAT |
| | | TTCCCGTAATTGACCGGAAACGATTATTACAACTAGTGAGAGAA |
| | | AATCAGCTGCAGTTAGATGAAAATGAGCTTCCTCACGCAGTTCAC |
| | | TTTCTAAATGAATCAGGAGTCCTTCTTCATTTTCAAGACCCAGCA |
| | | CTGCAGTTAAGTGACTTGTACTTTGTGGAACCCAAGTGGCTTTGT |
| | | AAAATCATGGCACAGTTTGTCTGACCACAGGCCTGTGATAGAGCT |
| | | TCCCCATTGTGAGAACTCTGAAATTATCATCCGACTATATGAAAT |
| | | GCCTTATTTTCCAATGGGATTTTGGTCAAGATTAAT |
| 10 | X5 | CCTGAGTGGGGGAGGAGGAAGCCGAGCAGGAGGGCTCCGGAGA |
| | | GGGAGGGCAACGCGGGGCGGGGAGCTGCCTCCTTCCTCATAAAC |
| | | AGGCGGGCGTGGGCGCCGATGGGGCCCGCGGGGAGCGCTGGCTG |
| | | CGGGCGGTGAGCTGAGCTCGCCCCCGGGGAGCTGTGGCCGGCGC |
| | | CCCTGCCGGTTCCCTGAGCAGCGGACGTTCATGCTGGGAGGGCG |
| | | GCGGGTTGGAAGCAGGTGCCACCATGGCTAGTGGCAGCTGTCAG |
| | | GGGTGCGAAGAGGACGAGGAAACTCTGAAGAAGTTGATAGTCAG |
| | | GCTGAACAATGTCCAGGAAGGAAAACAGATAGAAACGCTGGTCC |
| | | AAATCCTGGAGGATCTGCTGGTGTTCACGTACTCCGAGCGCGCCT |
| | | CCAAGTTATTTCAAGGCAAAAATATCCATGTGCCTCTGTTGATCG |
| | | TCTTGGACTCCTATATGAGAGTCGCGAGTGTGCAGCAGGTGGGTT |
| | | GGTCACTTCTGTGCAAATTAATAGAAGTCTGTCCAGGTACAATGC |
| | | AAAGCTTAATGGGACCCCAGGATGTTGGAAATGATTGGGAAGTC |
| | | CTTGGTGTTCACCAATTGATTCTTAAAATGCTAACAGTTCATAAT |
| | | GCCAGTGTAAACTTGTCAGTGATTGGACTGAAGACCTTAGATCTC |
| | | CTCCTAACTTCAGGTAAAATCACCTTGCTGATATTGGATGAAGAA |
| | | AGTGATATTTTCATGTTAATTTTTGATGCCATGCACTCATTTCCAG |
| | | CCAATGATGAAGTCCAGAAACTTGGATGCAAAGCTTTACATGTG |
| | | CTGTTTGAGAGAGTCTCAGAGGAGCAACTGACTGAATTTGTTGAG |
| | | AACAAAGATTATATGATATTGTTAAGTGCGTTAACAAATTTTAAA |
| | | GATGAAGAGGAAATTGTGCTTCATGTGCTGCATTGTTTACATTCC |
| | | CTAGCGATTCCTTGCAATAATGTGGAAGTCCTCATGAGTGGCAAT |
| | | GTCAGGTGTTATAATATTGTGGTGGAAGCTATGAAAGCATTCCCT |
| | | ATGAGTGAAAGAATTCAAGAAGTGAGTTGCTGTTTGCTCCATAG |
| | | GCTTACATTAGGTAATTTTTTCAATATCCTGGTATTAAACGAAGT |
| | | CCATGAGTTTGTGGTGAAAGCTGTGCAGCAGTACCCAGAGAATG |
| | | CAGCATTGCAGATCTCAGCGCTCAGCTGTTTGGCCCTCCTCACTG |
| | | AGACTATTTTCTTAAATCAAGATTTAGAGGAAAAGAATGAGAAT |
| | | CAAGAGAATGATGATGAGGGGGAAGAAGATAAATTGTTTTGGCT |
| | | GGAAGCCTGTTACAAAGCATTAACGTGGCATAGAAAGAACAAGC |
| | | ACGTGCAGGAGGCCGCATGCTGGGCACTAAATAATCTCCTTATGT |
| | | ACCAAAACAGTTTACATGAGAAGATTGGAGATGAAGATGGCCAT |
| | | TTCCCAGCTCATAGGGAAGTGATGCTCTCCATGCTGATGCATTCT |
| | | TCATCAAAGGAAGTTTTCCAGGCATCTGCGAATGCATTGTCAACT |
| | | CTCTTAGAACAAAATGTTAATTTCAGAAAAATACTGTTATCAAAA |
| | | GGAATACACCTGAATGTTTTGGAGTTAATGCAGAAGCATATACAT |
| | | TCTCCTGAAGTGGCTGAAAGTGGCTGTAAAATGCTAAATCATCTT |
| | | TTTGAAGGAAGCAACACTTCCCTGGATATAATGGCAGCAGTGGT |
| | | CCCCAAAATACTAACAGTTATGAAACGTCATGAGACATCATTACC |
| | | AGTGCAGCTGGAGGCGCTTCGAGCTATTTTACATTTTATAGTGCC |
| | | TGGCATGCCAGAAGAATCCAGGGAGGATACAGAATTTCATCATA |
| | | AGCTAAATATGGTTAAAAAACAGTGTTTCAAGAATGATATTCAC |
| | | AAACTGGTCCTAGCAGCTTTGAACAGGTTCATTGGAAATCCTGGG |
| | | ATTCAGAAATGTGGATTAAAAGTAATTTCTTCTATTGTACATTTTC |
| | | CTGATGCATTAGAGATGTTATCCCTGGAAGGTGCTATGGATTCAG |
| | | TGCTTCACACACTGCAGATGTATCCAGATGACCAAGAAATTCAGT |
| | | GTCTGGGTTTAAGTCTTATAGGATACTTGATTACAAAGAAGAATG |
| | | TGTTCATAGGAACTGGACATCTGCTGGCAAAAATTCTGGTTTCCA |
| | | GCTTATACCGATTTAAGGATGTTGCTGAAATACAGACTAAAGGAT |
| | | TTCAGACAATCTTAGCAATCCTCAAATTGTCAGCATCTTTTTCTAA |
| | | GCTGCTGGTGCATCATTCATTTGACTTAGTAATATTCCATCAAAT |
| | | GTCTTCCAATATCATGGAACAAAAGGATCAACAGTTTCTAAACCT |
| | | CTGTTGCAAGTGTTTTGCAAAAGTAGCTATGGATGATTACTTAAA |
| | | AAATGTGATGCTAGAGAGAGCGTGTGATCAGAATAACAGCATCA |
| | | TGGTTGAATGCTTGCTTCTATTGGGAGCAGATGCCAATCAAGCAA |
| | | AGGAGGGATCTTCTTTAATTTGTCAGGTATGTGAGAAAGAGAGC |
| | | AGTCCCAAATTGGTGGAACTCTTACTGAATAGTGGATCTCGTGAA |
| | | CAAGATGTACGAAAAGCGTTGACGATAAGCATTGGGAAAGGTGA |
| | | CAGCCAGATCATCAGCTTGCTCTTAAGGAGGCTGGCCCTGGATGT |
| | | GGCCAACAATAGCATTTGCCTTGGAGGATTTTGTATAGGAAAAGT |
| | | TGAACCTTCTTGGCTTGGTCCTTTATTTCCAGATAAGACTTCTAAT |
| | | TTAAGGAAACAAACAAATATAGCATCTACACTAGCAAGAATGGT |
| | | GATCAGATATCAGATGAAAAGTGCTGTGGAAGAAGGAACAGCCT |
| | | CAGGCAGCGATGGAAATTTTTCTGAAGATGTGCTGTCTAAATTTG |
| | | ATGAATGGACCTTTATTCCTGACTCTTCTATGGACAGTGTGTTTGC |
| | | TCAAAGTGATGACCTGGATAGTGAAGGAAGTGAAGGCTCATTTC |
| | | TTGTGAAAAAGAAATCTAATTCAATTAGTGTAGGAGAATTTTACC |
| | | GAGATGCCGTATTACAGCGTTGCTCACCAAATTTGCAAAGACATT |
| | | CCAATTCCTTGGGGCCCATTTTTGATCATGAAGATTTACTGAAGC |
| | | GAAAAAGAAAAATATTATCTTCAGATGATTCACTCAGGTCATCA |
| | | AAACTTCAATCCCATATGAGGCATTCAGACAGCATTTCTTCTCTG |
| | | GCTTCTGAGAGAGAATATATTACATCACTAGACCTTTCAGCAAAT |
| | | GAACTAAGAGATATTGATGCCCTAAGCCAGAAATGCTGTATAAG |
| | | TGTTCATTTGGAGCATCTTGAAAAGCTGGAGCTTCACCAGAATGC |
| | | ACTCACGAGCTTTCCACAACAGCTATGTGAAACTCTGAAGAGTTT |
| | | GACACATTTGGACTTGCACAGTAATAAATTTACATCATTTCCTTC |
| | | TTATTTGTTGAAAATGAGTTGTATTGCTAATCTTGATGTCTCTCGA |
| | | AATGACATTGGACCCTCAGTGGTTTTAGATCCTACAGTGAAATGT |
| | | CCAACTCTGAAACAGTTTAACCTGTCATATAACCAGCTGTCTTTT |
| | | GTACCTGAGAACCTCACTGATGTGGTAGAGAAACTGGAGCAGCT |
| | | CATTTTAGAAGGAAATAAAATATCAGGGATATGCTCCCCCTTGAG |
| | | ACTGAAGGAACTGAAGATTTTAAACCTTAGTAAGAACCACATTTC |
| | | ATCCCTATCAGAGAACTTTCTTGAGGCTTGTCCTAAAGTGGAGAG |
| | | TTTCAGTGCCAGAATGAATTTTCTTGCTGCTATGCCTTTCTTGCCT |
| | | CCTTCTATGACAATCCTAAAATTATCTCAGAACAAATTTTCCTGT |
| | | ATTCCAGAAGCAATTTTAAATCTTCCACACTTGCGGTCTTTAGAT |
| | | ATGAGCAGCAATGATATTCAGTACCTACCAGGTCCCGCACACTG |
| | | GAAATCTTTGAACTTAAGGGAACTCTTATTTAGCCATAATCAGAT |
| | | CAGCATCTTGGACTTGAGTGAAAAAGCATATTTATGGTCTAGAGT |
| | | AGAGAAACTGCATCTTTCTCACAATAAACTGAAAGAGATTCCTCC |
| | | TGAGATTGGCTGTCTTGAAAATCTGACATCTCTGGATGTCAGTTA |
| | | CAACTTGGAACTAAGATCCTTTCCCAATGAAATGGGGAAATTAA |
| | | GCAAAATATGGGATCTTCCTTTGGATGAACTGCATCTTAACTTTG |
| | | ATTTTAAACATATAGGATGTAAAGCCAAAGACATCATAAGGTTTC |
| | | TTCAACAGCGATTAAAAAAGGCTGTGCCTTATAACCGAATGAAA |
| | | CTTATGATTGTGGGAAATACTGGGAGTGGTAAAACCACCTTATTG |
| | | CAGCAATTAATGAAAACCAAGAAATCAGATCTTGGAATGCAAAG |
| | | TGCCACAGTTGGCATAGATGTGAAAGACTGGCCTATCCAAATAA |
| | | GAGACAAAAGAAAGAGAGATCTCGTCCTAAATGTGTGGGATTTT |
| | | GCAGGTCGTGAGGAATTCTATAGTACTCATCCCCATTTTATGACG |
| | | CAGCGAGCATTGTACCTTGCTGTCTATGACCTCAGCAAGGGACAG |
| | | GCTGAAGTTGATGCCATGAAGCCTTGGCTCTTCAATATAAAGGCT |
| | | CGCGCTTCTTCTTCCCCTGTGATTCTCGTTGGCACACATTTGGATG |
| | | TTTCTGATGAGAAGCAACGCAAAGCCTGCATGAGTAAAATCACC |
| | | AAGGAACTCCTGAATAAGCGAGGGTTCCCTGCCATACGAGATTA |
| | | CCACTTTGTGAATGCCACCGAGGAATCTGATGCTTTGGCAAAACT |
| | | TCGGAAAACCATCATAAACGAGAGCCTTAATTTCAAGGAGTCCTT |
| | | CTTCATTTTCAAGACCCAGCACTGCAGTTAAGTGACTTGTACTTT |
| | | GTGGAACCCAAGTGGCTTTGTAAAATCATGGCACAGG |
| 11 | X6 | GAAATCTAATTCAATTAGTGTAGGAGAATTTTACCGAGATGCCGT |
| | | ATTACAGCGTTGCTCACCAAATTTGCAAAGACATTCCAATTCCTT |
| | | GGGGCCCATTTTTGATCATGAAGATTTACTGAAGCGAAAAAGAA |
| | | AAATATTATCTTCAGATGATTCACTCAACTCTGAAGAGTTTGACA |
| | | CATTTGGACTTGCACAGTAATAAATTTACATCATTTCCTTCTTATT |
| | | TGTTGAAAATGAGTTGTATTGCTAATCTTGATGTCTCTCGAAATG |
| | | ACATTGGACCCTCAGTGGTTTTAGATCCTACAGTGAAATGTCCAA |
| | | CTCTGAAACAGTTTAACCTGTCATATAACCAGCTGTCTTTTGTAC |
| | | CTGAGAACCTCACTGATGTGGTAGAGAAACTGGAGCAGCTCATT |
| | | TTAGAAGGAAATAAAATATCAGGGATATGCTCCCCCTTGAGACT |
| | | GAAGGAACTGAAGATTTTAAACCTTAGTAAGAACCACATTTCATC |
| | | CCTATCAGAGAACTTTCTTGAGGCTTGTCCTAAAGTGGAGAGTTT |
| | | CAGTGCCAGAATGAATTTTCTTGCTGCTATGCCTTTCTTGCCTCCT |
| | | TCTATGACAATCCTAAAATTATCTCAGAACAAATTTTCCTGTATT |
| | | CCAGAAGCAATTTTAAATCTTCCACACTTGCGGTCTTTAGATATG |
| | | AGCAGCAATGATATTCAGTACCTACCAGGTCCCGCACACTGGAA |
| | | ATCTTTGAACTTAAGGGAACTCTTATTTAGCCATAATCAGATCAG |
| | | CATCTTGGACTTGAGTGAAAAAGCATATTTATGGTCTAGAGTAGA |
| | | GAAACTGCATCTTTCTCACAATAAACTGAAAGAGATTCCTCCTGA |
| | | GATTGGCTGTCTTGAAAATCTGACATCTCTGGATGTCAGTTACAA |
| | | CTTGGAACTAAGATCCTTTCCCAATGAAATGGGGAAATTAAGCA |
| | | AAATATGGGATCTTCCTTTGGATGAACTGCATCTTAACTTTGATTT |
| | | TAAACATATAGGATGTAAAGCCAAAGACATCATAAGGTTTCTTC |
| | | AACAGCGATTAAAAAAGGCTGTGCCTTATAACCGAATGAAACTT |
| | | ATGATTGTGGGAAATACTGGGAGTGGTAAAACCACCTTATTGCA |
| | | GCAATTAATGAAAACCAAGAAATCAGATCTTGGAATGCAAAGTG |
| | | CCACAGTTGGCATAGATGTGAAAGACTGGCCTATCCAAATAAGA |
| | | GACAAAAGAAAGAGAGATCTCGTCCTAAATGTGTGGGATTTTGC |
| | | AGGTCGTGAGGAATTCTATAGTACTCATCCCCATTTTATGACGCA |
| | | GCGAGCATTGTACCTTGCTGTCTATGACCTCAGCAAGGGACAGGC |
| | | TGAAGTTGATGCCATGAAGCCTTGGCTCTTCAATATAAAGGCTCG |
| | | CGCTTCTTCTTCCCCTGTGATTCTCGTTGGCACACATTTGGATGTT |
| | | TCTGATGAGAAGCAACGCAAAGCCTGCATGAGTAAAATCACCAA |
| | | GGAACTCCTGAATAAGCGAGGGTTCCCTGCCATACGAGATTACC |
| | | ACTTTGTGAATGCCACCGAGGAATCTGATGCTTTGGCAAAACTTC |
| | | GGAAAACCATCATAAACGAGAGCCTTAATTTCAAGATCCGAGAT |
| | | CAGCTTGTTGTTGGACAGCTGATTCCAGACTGCTATGTAGAACTT |
| | | GAAAAAATCATTTTATCGGAGCGTAAAAATGTGCCAATTGAATTT |
| | | CCCGTAATTGACCGGAAACGATTATTACAACTAGTGAGAGAAAA |
| | | TCAGCTGCAGTTAGATGAAAATGAGCTTCCTCACGCAGTTCACTT |
| | | TCTAAATGAATCAGGAGTCCTTCTTCATTTTCAAGACCCAGCACT |
| | | GCAGTTAAGTGACTTGTACTTTGTGGAACCCAAGTGGCTTTGTAA |
| | | AATCATGGCACAGATTTTGACAGTGAAAGTGGAAGGTTGTCCAA |
| | | AACACCCTAAGGGCATTATTTCGCGTAGAGATGTGGAAAAATTTC |
| | | TTTCAAAAAAAAGGAAATTTCCAAAGAACTACATGTCACAGTAT |
| | | TTTAAGCTCCTAGAAAAATTCCAGATTGCTTTGCCAATAGGAGAA |
| | | GAATATTTGCTGGTTCCAAGCAGTTTGTCTGACCACAGGCCTGTG |
| | | ATAGAGCTTCCCCATTGTGAGAACTCTGAAATTATCATCCGACTA |
| | | TATGAAATGCCTTATTTTCCAATGGGATTTTGGTCAAGATTAATC |
| | | AATCGATTACTTGAGATTTCACCTTACATGCTTTCAGGGAGAGAA |
| | | CGAGCACTTCGCCCAAACAGAATGTATTGGCGACAAGGCATTTA |
| | | CTTAAATTGGTCTCCTGAAGCTTATTGTCTGGTAGGATCTGAAGT |
| | | CTTAGACAATCATCCAGAGAGTTTCTTAAAAATTACAGTTCCTTC |
| | | TTGTAGAAAAGGCTGTATTCTTTTGGGCCAAGTTGTGGACCACAT |
| | | TGATTCTCTCATGGAAGAATGGTTTCCTGGGTTGCTGGAGATTGA |
| | | TATTTGTGGTGAAGGAGAAACTCTGTTGAAGAAATGGGCATTAT |
| | | ATAGTTTTAATGATGGTGAAGAACATCAAAAAATCTTACTTGATG |
| | | ACTTGATGAAGAAAGCAGAGGAAGGAGATCTCTTAGTAAATCCA |
| | | GATCAACCAAGGCTCACCATTCCAATATCTCAGATTGCCCCTGAC |
| | | TTGATTTTGGCTGACCTGCCTAGAAATATTATGTTGAATAATGAT |
| | | GAGTTGGAATTTGAACAAGCTCCAGAGTTTCTCCTAGGTGATGGC |
| | | AGTTTTGGATCAGTTTACCGAGCAGCCTATGAAGGAGAAGAAGT |
| | | GGCTGTGAAGATTTTTAATAAACATACATCACTCAGGCTGTTAAG |
| | | ACAAGAGCTTGTGGTGCTTTGCCACCTCCACCACCCCAGTTTGAT |
| | | ATCTTTGCTGGCAGCTGGGATTCGTCCCCGGATGTTGGTGATGGA |
| | | GTTAGCCTCCAAGGGTTCCTTGGATCGCCTGCTTCAGCAGGACAA |
| | | AGCCAGCCTCACTAGAACCCTACAGCACAGGATTGCACTCCACG |
| | | TAGCTGATGGTTTGAGATACCTCCACTCAGCCATGATTATATACC |
| | | GAGACCTGAAACCCCACAATGTGCTGCTTTTCACACTGTATCCCA |
| | | ATGCTGCCATCATTGCAAAGATTGCTGACTACGGCATTGCTCAGT |
| | | ACTGCTGTAGAATGGGGATAAAAACATCAGAGGGCACACCAGGG |
| | | TTTCGTGCACCTGAAGTTGCCAGAGGAAATGTCATTTATAACCAA |
| | | CAGGCTGATGTTTATTCATTTGGTTTACTACTCTATGACATTTTGA |
| | | CAACTGGAGGTAGAATAGTAGAGGGTTTGAAGTTTCCAAATGAG |
| | | TTTGATGAATTAGAAATACAAGGAAAATTACCTGATCCAGTTAA |
| | | AGAATATGGTTGTGCCCCATGGCCTATGGTTGAGAAATTAATTAA |
| | | ACAGTGTTTGAAAGAAAATCCTCAAGAAAGGCCTACTTCTGCCC |
| | | AGGTCTTTGACATTTTGAATTCAGCTGAATTAGTCTGTCTGACGA |
| | | GACGCATTTTATTACCTAAAAACGTAATTGTTGAATGCATGGTTG |
| | | CTACACATCACAACAGCAGGAATGCAAGCATTTGGCTGGGCTGT |
| | | GGGCACACCGACAGAGGACAGCTCTCATTTCTTGACTTAAATACT |
| | | GAAGGATACACTTCTGAGGAAGTTGCTGATAGTAGAATATTGTG |
| | | CTTAGCCTTGGTGCATCTTCCTGTTGAAAAGGAAAGCTGGATTGT |
| | | GTCTGGGACACAGTCTGGTACTCTCCTGGTCATCAATACCGAAGA |
| | | TGGGAAAAAGAGACATACCCTAGAAAAGATGACTGATTCTGTCA |
| | | CTTGTTTGTATTGCAATTCCTTTTCCAAGCAAAGCAAACAAAAAA |
| | | ATTTTCTTTTGGTTGGAACCGCTGATGGCAAGTTAGCAATTTTTG |
| | | AAGATAAGACTGTTAAGCTTAAAGGAGCTGCTCCTTTGAAGATA |
| | | CTAAATATAGGAAATGTCAGTACTCCATTGATGTGTTTGAGTGAA |
| | | TCCACAAATTCAACGGAAAGAAATGTAATGTGGGGAGGATGTGG |
| | | CACAAAGATTTTCTCCTTTTCTAATGATTTCACCATTCAGAAACTC |
| | | ATTGAGACAAGAACAAGCCAACTGTTTTCTTATGCAGCTTTCAGT |
| | | GATTCCAACATCATAACAGTGGTGGTAGACACTGCTCTCTATATT |
| | | GCTAAGCAAAATAGCCCTGTTGTGGAAGTGTGGGATAAGAAAAC |
| | | TGAAAAACTCTGTGGACTAATAGACTGCGTGCACTTTTTAAGGGA |
| | | GGTAATGGTAAAAGAAAACAAGGAATCAAAACACAAAATGTCTT |
| | | ATTCTGGGAGAGTGAAAACCCTCTGCCTTCAGAAGAACACTGCTC |
| | | TTTGGATAGGAACTGGAGGAGGCCATATTTTACTCCTGGATCTTT |
| | | CAACTCGTCGACTTATACGTGTAATTTACAACTTTTGTAATTCGGT |
| | | CAGAGTCATGATGACAGCACAGCTAGGAAGCCTTAAAAATGTCA |
| | | TGCTGGTATTGGGCTACAACCGGAAAAATACTGAAGGTACACAA |
| | | AAGCAGAAAGAGATACAATCTTGCTTGACCGTTTGGGACATCAA |
| | | TCTTCCACATGAAGTGCAAAATTTAGAAAAACACATTGAAGTGA |
| | | GAAAAGAATTAGCTGAAAAAATGAGACGAACATCTGTTGAGTAA |
| | | GAGAGAAATAGGAATTGTCTTTGGATAGGAAAATTATTCTCTCCT |
| | | CTTGTAAATATTTATTTTAAAAATGTTCACATGGAAAGGGTACTC |
| | | ACATTTTTTGAAATAGCTCGTGTGTATGAAGGAATGTTATTATTTT |
| | | TAATTTAAATATATGTAAAAATACTTACCAGTAAATGTGTATTTT |
| | | AAAGAACTATTTAAAACACAATGTTATATTTCTTATAAATACCAG |
| | | TTACTTTCGTTCATTAATTAATGAAAATAAATCTGTGAAGTACCT |
| | | AATTTAAGTACTCATACTAAAATTTATAAGGCCGATAATTTTTTG |
| | | TTTTCTTGTCTGTAATGGAGGTAAACTTTATTTTAAATTCTGTGCT |
| | | TAAGACAGGACTATTGCTTGTCGATTTTTCTAGAAATCTGCACGG |
| | | TATAATGAAAATATTAAGACAGTTTCCCATGTAATGTATTCCTTC |
| | | TTAGATTGCATCGAAATGCACTATCATATATGCTTGTAAATATTC |
| | | AAATGAATTTGCACTAATAAAGTCCTTTGTTGGTATGTGAATTCT |
| | | CTTTGTTGCTGTTGCAAACAGTGCATCTTACACAACTTCACTCAA |
| | | TTCAAAAGAAAACTCCATTAAAAGTACTAATGAAAAAACATGAC |
| | | ATACTGTCAAAGTCCTCATATCTAGGAAAGACACAGAAACTCTCT |
| | | TTGTCACAGAAACTCTCTGTGTCTTTCCTAGACATAATAGAGTTG |
| | | TTTTTCAACTCTATGTTTGAATGTGGATACCCTGAATTTTGTATAA |
| | | TTAGTGTAAATACAGTGTTCAGTCCTTCAAGTGATATTTTTATTTT |
| | | TTTATTCATACCACTAGCTACTTGTTTTCTAATCTGCTTCATTCTA |
| | | ATGCTTATATTCATCTTTTCCCTAAATTTGTGATGCTGCAGATCCT |
| | | ACATCATTCAGATAGAAACCTTTTTTTTTTTCAGAATTATAGAATT |
| | | CCACAGCTCCTACCAAGACCATGAGGATAAATATCTAACACTTTT |
| | | CAGTTGCTGAAGGAGAAAGGAGCTTTAGTTATGATGGATAAAAA |
| | | TATCTGCCACCCTAGGCTTCCAAATTATACTTAAATTGTTTACATA |
| | | GCTTACCACAATAGGAGTATCAGGGCCAAATACCTATGTAATAA |
| | | TTTGAGGTCATTTCTGCTTTAGGAAAAGTACTTTCGGTAAATTCTT |
| | | TGGCCCTGACCAGTATTCATTATTTCAGATAATTCCCTGTGATAG |
| | | GACAACTAGTACATTTAATATTCTCAGAACTTATGGCATTTTACT |
| | | ATGTGAAAACTTTAAATTTATTTATATTAAGGGTAATCAAATTCT |
| | | TAAAGATGAAAGATTTTCTGTATTTTAAAGGAAGCTATGCTTTAA |
| | | CTTGTTATGTAATTAACAAAAAAATCATATATAATAGAGCTCTTT |
| | | GTTCCAGTGTTATCTCTTTCATTGTTACTTTGTATTTGCAATTTTTT |
| | | TTACCAAAGACAAATTAAAAAAATGAATACCATATTTAAATGGA |
| | | ATAATAAAGGTTTTTTAAAAACTT |
| 12 | X7 | GAGTAGAGAAACTGCATCTTTCTCACAATAAACTGAAAGAGGAG |
| | | CAGAATGAAGTCACAGATAATGGATAACCAAATCCATTTTGTGA |
| | | TTCCTCCTGAGATTGGCTGTCTTGAAAATCTGACATCTCTGGATG |
| | | TCAGTTACAACTTGGAACTAAGATCCTTTCCCAATGAAATGGGGA |
| | | AATTAAGCAAAATATGGGATCTTCCTTTGGATGAACTGCATCTTA |
| | | ACTTTGATTTTAAACATATAGGATGTAAAGCCAAAGACATCATAA |
| | | GGTTTCTTCAACAGCGATTAAAAAAGGCTGTGCCTTATAACCGAA |
| | | TGAAACTTATGATTGTGGGAAATACTGGGAGTGGTAAAACCACC |
| | | TTATTGCAGCAATTAATGAAAACCAAGAAATCAGATCTTGGAAT |
| | | GCAAAGTGCCACAGTTGGCATAGATGTGAAAGACTGGCCTATCC |
| | | AAATAAGAGACAAAAGAAAGAGAGATCTCGTCCTAAATGTGTGG |
| | | GATTTTGCAGGTCGTGAGGAATTCTATAGTACTCATCCCCATTTT |
| | | ATGACGCAGCGAGCATTGTACCTTGCTGTCTATGACCTCAGCAAG |
| | | GGACAGGCTGAAGTTGATGCCATGAAGCCTTGGCTCTTCAATATA |
| | | AAGGCTCGCGCTTCTTCTTCCCCTGTGATTCTCGTTGGCACACATT |
| | | TGGATGTTTCTGATGAGAAGCAACGCAAAGCCTGCATGAGTAAA |
| | | ATCACCAAGGAACTCCTGAATAAGCGAGGGTTCCCTGCCATACG |
| | | AGATTACCACTTTGTGAATGCCACCGAGGAATCTGATGCTTTGGC |
| | | AAAACTTCGGAAAACCATCATAAACGAGAGCCTTAATTTCAAGA |
| | | TCCGAGATCAGCTTGTTGTTGGACAGCTGATTCCAGACTGCTATG |
| | | TAGAACTTGAAAAAATCATTTTATCGGAGCGTAAAAATGTGCCA |
| | | ATTGAATTTCCCGTAATTGACCGGAAACGATTATTACAACTAGTG |
| | | AGAGAAAATCAGCTGCAGTTAGATGAAAATGAGCTTCCTCACGC |
| | | AGTTCACTTTCTAAATGAATCAGGAGTCCTTCTTCATTTTCAAGA |
| | | CCCAGCACTGCAGTTAAGTGACTTGTACTTTGTGGAACCCAAGTG |
| | | GCTTTGTAAAATCATGGCACAGATTTTGACAGTGAAAGTGGAAG |
| | | GTTGTCCAAAACACCCTAAGGGCATTATTTCGCGTAGAGATGTGG |
| | | AAAAATTTCTTTCAAAAAAAAGGAAATTTCCAAAGAACTACATG |
| | | TCACAGTATTTTAAGCTCCTAGAAAAATTCCAGATTGCTTTGCCA |
| | | ATAGGAGAAGAATATTTGCTGGTTCCAAGCAGTTTGTCTGACCAC |
| | | AGGCCTGTGATAGAGCTTCCCCATTGTGAGAACTCTGAAATTATC |
| | | ATCCGACTATATGAAATGCCTTATTTTCCAATGGGATTTTGGTCA |
| | | AGATTAATCAATCGATTACTTGAGATTTCACCTTACATGCTTTCA |
| | | GGGAGAGAACGAGCACTTCGCCCAAACAGAATGTATTGGCGACA |
| | | AGGCATTTACTTAAATTGGTCTCCTGAAGCTTATTGTCTGGTAGG |
| | | ATCTGAAGTCTTAGACAATCATCCAGAGAGTTTCTTAAAAATTAC |
| | | AGTTCCTTCTTGTAGAAAAGGCTGTATTCTTTTGGGCCAAGTTGT |
| | | GGACCACATTGATTCTCTCATGGAAGAATGGTTTCCTGGGTTGCT |
| | | GGAGATTGATATTTGTGGTGAAGGAGAAACTCTGTTGAAGAAAT |
| | | GGGCATTATATAGTTTTAATGATGGTGAAGAACATCAAAAAATCT |
| | | TACTTGATGACTTGATGAAGAAAGCAGAGGAAGGAGATCTCTTA |
| | | GTAAATCCAGATCAACCAAGGCTCACCATTCCAATATCTCAGATT |
| | | GCCCCTGACTTGATTTTGGCTGACCTGCCTAGAAATATTATGTTG |
| | | AATAATGATGAGTTGGAATTTGAACAAGCTCCAGAGTTTCTCCTA |
| | | GGTGATGGCAGTTTTGGATCAGTTTACCGAGCAGCCTATGAAGG |
| | | AGAAGAAGTGGCTGTGAAGATTTTTAATAAACATACATCACTCA |
| | | GGCTGTTAAGACAAGAGCTTGTGGTGCTTTGCCACCTCCACCACC |
| | | CCAGTTTGATATCTTTGCTGGCAGCTGGGATTCGTCCCCGGATGT |
| | | TGGTGATGGAGTTAGCCTCCAAGGGTTCCTTGGATCGCCTGCTTC |
| | | AGCAGGACAAAGCCAGCCTCACTAGAACCCTACAGCACAGGATT |
| | | GCACTCCACGTAGCTGATGGTTTGAGATACCTCCACTCAGCCATG |
| | | ATTATATACCGAGACCTGAAACCCCACAATGTGCTGCTTTTCACA |
| | | CTGTATCCCAATGCTGCCATCATTGCAAAGATTGCTGACTACGGC |
| | | ATTGCTCAGTACTGCTGTAGAATGGGGATAAAAACATCAGAGGG |
| | | CACACCAGGGTTTCGTGCACCTGAAGTTGCCAGAGGAAATGTCA |
| | | TTTATAACCAACAGGCTGATGTTTATTCATTTGGTTTACTACTCTA |
| | | TGACATTTTGACAACTGGAGGTAGAATAGTAGAGGGTTTGAAGT |
| | | TTCCAAATGAGTTTGATGAATTAGAAATACAAGGAAAATTACCT |
| | | GATCCAGTTAAAGAATATGGTTGTGCCCCATGGCCTATGGTTGAG |
| | | AAATTAATTAAACAGTGTTTGAAAGAAAATCCTCAAGAAAGGCC |
| | | TACTTCTGCCCAGGTCTTTGACATTTTGAATTCAGCTGAATTAGTC |
| | | TGTCTGACGAGACGCATTTTATTACCTAAAAACGTAATTGTTGAA |
| | | TGCATGGTTGCTACACATCACAACAGCAGGAATGCAAGCATTTG |
| | | GCTGGGCTGTGGGCACACCGACAGAGGACAGCTCTCATTTCTTGA |
| | | CTTAAATACTGAAGGATACACTTCTGAGGAAGTTGCTGATAGTAG |
| | | AATATTGTGCTTAGCCTTGGTGCATCTTCCTGTTGAAAAGGAAAG |
| | | CTGGATTGTGTCTGGGACACAGTCTGGTACTCTCCTGGTCATCAA |
| | | TACCGAAGATGGGAAAAAGAGACATACCCTAGAAAAGATGACTG |
| | | ATTCTGTCACTTGTTTGTATTGCAATTCCTTTTCCAAGCAAAGCAA |
| | | ACAAAAAAATTTTCTTTTGGTTGGAACCGCTGATGGCAAGTTAGC |
| | | AATTTTTGAAGATAAGACTGTTAAGCTTAAAGGAGCTGCTCCTTT |
| | | GAAGATACTAAATATAGGAAATGTCAGTACTCCATTGATGTGTTT |
| | | GAGTGAATCCACAAATTCAACGGAAAGAAATGTAATGTGGGGAG |
| | | GATGTGGCACAAAGATTTTCTCCTTTTCTAATGATTTCACCATTCA |
| | | GAAACTCATTGAGACAAGAACAAGCCAACTGTTTTCTTATGCAGC |
| | | TTTCAGTGATTCCAACATCATAACAGTGGTGGTAGACACTGCTCT |
| | | CTATATTGCTAAGCAAAATAGCCCTGTTGTGGAAGTGTGGGATAA |
| | | GAAAACTGAAAAACTCTGTGGACTAATAGACTGCGTGCACTTTTT |
| | | AAGGGAGGTAATGGTAAAAGAAAACAAGGAATCAAAACACAAA |
| | | ATGTCTTATTCTGGGAGAGTGAAAACCCTCTGCCTTCAGAAGAAC |
| | | ACTGCTCTTTGGATAGGAACTGGAGGAGGCCATATTTTACTCCTG |
| | | GATCTTTCAACTCGTCGACTTATACGTGTAATTTACAACTTTTGTA |
| | | ATTCGGTCAGAGTCATGATGACAGCACAGCTAGGAAGCCTTAAA |
| | | AATGTCATGCTGGTATTGGGCTACAACCGGAAAAATACTGAAGG |
| | | TACACAAAAGCAGAAAGAGATACAATCTTGCTTGACCGTTTGGG |
| | | ACATCAATCTTCCACATGAAGTGCAAAATTTAGAAAAACACATT |
| | | GAAGTGAGAAAAGAATTAGCTGAAAAAATGAGACGAACATCTGT |
| | | TGAGTAAGAGAGAAATAGGAATTGTCTTTGGATAGGAAAATTAT |
| | | TCTCTCCTCTTGTAAATATTTATTTTAAAAATGTTCACATGGAAAG |
| | | GGTACTCACATTTTTTGAAATAGCTCGTGTGTATGAAGGAATGTT |
| | | ATTATTTTTAATTTAAATATATGTAAAAATACTTACCAGTAAATG |
| | | TGTATTTTAAAGAACTATTTAAAACACAATGTTATATTTCTTATA |
| | | AATACCAGTTACTTTCGTTCATTAATTAATGAAAATAAATCTGTG |
| | | AAGTACCTAATTTAAGTACTCATACTAAAATTTATAAGGCCGATA |
| | | ATTTTTTGTTTTCTTGTCTGTAATGGAGGTAAACTTTATTTTAAAT |
| | | TCTGTGCTTAAGACAGGACTATTGCTTGTCGATTTTTCTAGAAAT |
| | | CTGCACGGTATAATGAAAATATTAAGACAGTTTCCCATGTAATGT |
| | | ATTCCTTCTTAGATTGCATCGAAATGCACTATCATATATGCTTGTA |
| | | AATATTCAAATGAATTTGCACTAATAAAGTCCTTTGTTGGTATGT |
| | | GAATTCTCTTTGTTGCTGTTGCAAACAGTGCATCTTACACAACTTC |
| | | ACTCAATTCAAAAGAAAACTCCATTAAAAGTACTAATGAAAAAA |
| | | CATGACATACTGTCAAAGTCCTCATATCTAGGAAAGACACAGAA |
| | | ACTCTCTTTGTCACAGAAACTCTCTGTGTCTTTCCTAGACATAATA |
| | | GAGTTGTTTTTCAACTCTATGTTTGAATGTGGATACCCTGAATTTT |
| | | GTATAATTAGTGTAAATACAGTGTTCAGTCCTTCAAGTGATATTT |
| | | TTATTTTTTTATTCATACCACTAGCTACTTGTTTTCTAATCTGCTTC |
| | | ATTCTAATGCTTATATTCATCTTTTCCCTAAATTTGTGATGCTGCA |
| | | GATCCTACATCATTCAGATAGAAACCTTTTTTTTTTTCAGAATTAT |
| | | AGAATTCCACAGCTCCTACCAAGACCATGAGGATAAATATCTAA |
| | | CACTTTTCAGTTGCTGAAGGAGAAAGGAGCTTTAGTTATGATGGA |
| | | TAAAAATATCTGCCACCCTAGGCTTCCAAATTATACTTAAATTGT |
| | | TTACATAGCTTACCACAATAGGAGTATCAGGGCCAAATACCTATG |
| | | TAATAATTTGAGGTCATTTCTGCTTTAGGAAAAGTACTTTCGGTA |
| | | AATTCTTTGGCCCTGACCAGTATTCATTATTTCAGATAATTCCCTG |
| | | TGATAGGACAACTAGTACATTTAATATTCTCAGAACTTATGGCAT |
| | | TTTACTATGTGAAAACTTTAAATTTATTTATATTAAGGGTAATCA |
| | | AATTCTTAAAGATGAAAGATTTTCTGTATTTTAAAGGAAGCTATG |
| | | CTTTAACTTGTTATGTAATTAACAAAAAAATCATATATAATAGAG |
| | | CTCTTTGTTCCAGTGTTATCTCTTTCATTGTTACTTTGTATTTGCAA |
| | | TTTTTTTTACCAAAGACAAATTAAAAAAATGAATACCATATTTAA |
| | | ATGGAATAATAAAGGTTTTTTAAAAACTT |
| 13 | X8 | CCTGAGTGGGGGAGGAGGAAGCCGAGCAGGAGGGCTCCGGAGA |
| | | GGGAGGGCAACGCGGGGCGGGGAGCTGCCTCCTTCCTCATAAAC |
| | | AGGCGGGCGTGGGCGCCGATGGGGCCCGCGGGGAGCGCTGGCTG |
| | | CGGGCGGTGAGCTGAGCTCGCCCCCGGGGAGCTGTGGCCGGCGC |
| | | CCCTGCCGGTTCCCTGAGCAGCGGACGTTCATGCTGGGAGGGCG |
| | | GCGGGTTGGAAGCAGGTGCCACCATGGCTAGTGGCAGCTGTCAG |
| | | GGGTGCGAAGAGGACGAGGAAACTCTGAAGAAGTTGATAGTCAG |
| | | GCTGAACAATGTCCAGGAAGGAAAACAGATAGAAACGCTGGTCC |
| | | AAATCCTGGAGGATCTGCTGGTGTTCACGTACTCCGAGCGCGCCT |
| | | CCAAGTTATTTCAAGGCAAAAATATCCATGTGCCTCTGTTGATCG |
| | | TCTTGGACTCCTATATGAGAGTCGCGAGTGTGCAGCAGGTGGGTT |
| | | GGTCACTTCTGTGCAAATTAATAGAAGTCTGTCCAGGTACAATGC |
| | | AAAGCTTAATGGGACCCCAGGATGTTGGAAATGATTGGGAAGTC |
| | | CTTGGTGTTCACCAATTGATTCTTAAAATGCTAACAGTTCATAAT |
| | | GCCAGTGTAAACTTGTCAGTGATTGGACTGAAGACCTTAGATCTC |
| | | CTCCTAACTTCAGGTAAAATCACCTTGCTGATATTGGATGAAGAA |
| | | AGTGATATTTTCATGTTAATTTTTGATGCCATGCACTCATTTCCAG |
| | | CCAATGATGAAGTCCAGAAACTTGGATGCAAAGCTTTACATGTG |
| | | CTGTTTGAGAGAGTCTCAGAGGAGCAACTGACTGAATTTGTTGAG |
| | | AACAAAGATTATATGATATTGTTAAGTGCGTTAACAAATTTTAAA |
| | | GATGAAGAGGAAATTGTGCTTCATGTGCTGCATTGTTTACATTCC |
| | | CTAGCGATTCCTTGCAATAATGTGGAAGTCCTCATGAGTGGCAAT |
| | | GTCAGGTGTTATAATATTGTGGTGGAAGCTATGAAAGCATTCCCT |
| | | ATGAGTGAAAGAATTCAAGAAGTGAGTTGCTGTTTGCTCCATAG |
| | | GCTTACATTAGGTAATTTTTTCAATATCCTGGTATTAAACGAAGT |
| | | CCATGAGTTTGTGGTGAAAGCTGTGCAGCAGTACCCAGAGAATG |
| | | CAGCATTGCAGATCTCAGCGCTCAGCTGTTTGGCCCTCCTCACTG |
| | | AGACTATTTTCTTAAATCAAGATTTAGAGGAAAAGAATGAGAAT |
| | | CAAGAGAATGATGATGAGGGGGAAGAAGATAAATTGTTTTGGCT |
| | | GGAAGCCTGTTACAAAGCATTAACGTGGCATAGAAAGAACAAGC |
| | | ACGTGCAGGAGGCCGCATGCTGGGCACTAAATAATCTCCTTATGT |
| | | ACCAAAACAGTTTACATGAGAAGATTGGAGATGAAGATGGCCAT |
| | | TTCCCAGCTCATAGGGAAGTGATGCTCTCCATGCTGATGCATTCT |
| | | TCATCAAAGGAAGTTTTCCAGGCATCTGCGAATGCATTGTCAACT |
| | | CTCTTAGAACAAAATGTTAATTTCAGAAAAATACTGTTATCAAAA |
| | | GGAATACACCTGAATGTTTTGGAGTTAATGCAGAAGCATATACAT |
| | | TCTCCTGAAGTGGCTGAAAGTGGCTGTAAAATGCTAAATCATCTT |
| | | TTTGAAGGAAGCAACACTTCCCTGGATATAATGGCAGCAGTGGT |
| | | CCCCAAAATACTAACAGTTATGAAACGTCATGAGACATCATTACC |
| | | AGTGCAGCTGGAGGCGCTTCGAGCTATTTTACATTTTATAGTGCC |
| | | TGGCATGCCAGAAGAATCCAGGGAGGATACAGAATTTCATCATA |
| | | AGCTAAATATGGTTAAAAAACAGTGTTTCAAGAATGATATTCAC |
| | | AAACTGGTCCTAGCAGCTTTGAACAGGTTCATTGGAAATCCTGGG |
| | | ATTCAGAAATGTGGATTAAAAGTAATTTCTTCTATTGTACATTTTC |
| | | CTGATGCATTAGAGATGTTATCCCTGGAAGGTGCTATGGATTCAG |
| | | TGCTTCACACACTGCAGATGTATCCAGATGACCAAGAAATTCAGT |
| | | GTCTGGGTTTAAGTCTTATAGGATACTTGATTACAAAGAAGAATG |
| | | TGTTCATAGGAACTGGACATCTGCTGGCAAAAATTCTGGTTTCCA |
| | | GCTTATACCGATTTAAGGATGTTGCTGAAATACAGACTAAAGGAT |
| | | TTCAGACAATCTTAGCAATCCTCAAATTGTCAGCATCTTTTTCTAA |
| | | GCTGCTGGTGCATCATTCATTTGACTTAGTAATATTCCATCAAAT |
| | | GTCTTCCAATATCATGGAACAAAAGGATCAACAGTTTCTAAACCT |
| | | CTGTTGCAAGTGTTTTGCAAAAGTAGCTATGGATGATTACTTAAA |
| | | AAATGTGATGCTAGAGAGAGCGTGTGATCAGAATAACAGCATCA |
| | | TGGTTGAATGCTTGCTTCTATTGGGAGCAGATGCCAATCAAGCAA |
| | | AGGAGGGATCTTCTTTAATTTGTCAGGTATGTGAGAAAGAGAGC |
| | | AGTCCCAAATTGGTGGAACTCTTACTGAATAGTGGATCTCGTGAA |
| | | CAAGATGTACGAAAAGCGTTGACGATAAGCATTGGGAAAGGTGA |
| | | CAGCCAGATCATCAGCTTGCTCTTAAGGAGGCTGGCCCTGGATGT |
| | | GGCCAACAATAGCATTTGCCTTGGAGGATTTTGTATAGGAAAAGT |
| | | TGAACCTTCTTGGCTTGGTCCTTTATTTCCAGATAAGACTTCTAAT |
| | | TTAAGGAAACAAACAAATATAGCATCTACACTAGCAAGAATGGT |
| | | GATCAGATATCAGATGAAAAGTGCTGTGGAAGAAGGAACAGCCT |
| | | CAGGCAGCGATGGAAATTTTTCTGAAGATGTGCTGTCTAAATTTG |
| | | ATGAATGGACCTTTATTCCTGACTCTTCTATGGACAGTGTGTTTGC |
| | | TCAAAGTGATGACCTGGATAGTGAAGGAAGTGAAGGCTCATTTC |
| | | TTGTGAAAAAGAAATCTAATTCAATTAGTGTAGGAGAATTTTACC |
| | | GAGATGCCGTATTACAGCGTTGCTCACCAAATTTGCAAAGACATT |
| | | CCAATTCCTTGGGGCCCATTTTTGATCATGAAGATTTACTGAAGC |
| | | GAAAAAGAAAAATATTATCTTCAGATGATTCACTCAGGTCATCA |
| | | AAACTTCAATCCCATATGAGGCATTCAGACAGCATTTCTTCTCTG |
| | | GCTTCTGAGAGAGAATATATTACATCACTAGACCTTTCAGCAAAT |
| | | GAACTAAGAGATATTGATGCCCTAAGCCAGAAATGCTGTATAAG |
| | | TGTTCATTTGGAGCATCTTGAAAAGCTGGAGCTTCACCAGAATGC |
| | | ACTCACGAGCTTTCCACAACAGCTATGTGAAACTCTGAAGAGTTT |
| | | GACACATTTGGACTTGCACAGTAATAAATTTACATCATTTCCTTC |
| | | TTATTTGTTGAAAATGAGTTGTATTGCTAATCTTGATGTCTCTCGA |
| | | AATGACATTGGACCCTCAGTGGTTTTAGATCCTACAGTGAAATGT |
| | | CCAACTCTGAAACAGTTTAACCTGTCATATAACCAGCTGTCTTTT |
| | | GTACCTGAGAACCTCACTGATGTGGTAGAGAAACTGGAGCAGCT |
| | | CATTTTAGAAGGAAATAAAATATCAGGGATATGCTCCCCCTTGAG |
| | | ACTGAAGGAACTGAAGATTTTAAACCTTAGTAAGAACCACATTTC |
| | | ATCCCTATCAGAGAACTTTCTTGAGGCTTGTCCTAAAGTGGAGAG |
| | | TTTCAGTGCCAGAATGAATTTTCTTGCTGCTATGCCTTTCTTGCCT |
| | | CCTTCTATGACAATCCTAAAATTATCTCAGAACAAATTTTCCTGT |
| | | ATTCCAGAAGCAATTTTAAATCTTCCACACTTGCGGTCTTTAGAT |
| | | ATGAGCAGCAATGATATTCAGTACCTACCAGGTCCCGCACACTG |
| | | GAAATCTTTGAACTTAAGGGAACTCTTATTTAGCCATAATCAGAT |
| | | CAGCATCTTGGACTTGAGTGAAAAAGCATATTTATGGTCTAGAGT |
| | | AGAGAAACTGCATCTTTCTCACAATAAACTGAAAGAGGTTTCTTC |
| | | AACAGCGATTAAAAAAGGCTGTGCCTTATAACCGAATGAAACTT |
| | | ATGATTGTGGGAAATACTGGGAGTGGTAAAACCACCTTATTGCA |
| | | GCAATTAATGAAAACCAAGAAATCAGATCTTGGAATGC |
| 14 | X9 | CCTGAGTGGGGGAGGAGGAAGCCGAGCAGGAGGGCTCCGGAGA |
| | | GGGAGGGCAACGCGGGGCGGGGAGCTGCCTCCTTCCTCATAAAC |
| | | AGGCGGGCGTGGGCGCCGATGGGGCCCGCGGGGAGCGCTGGCTG |
| | | CGGGCGGTGAGCTGAGCTCGCCCCCGGGGAGCTGTGGCCGGCGC |
| | | CCCTGCCGGTTCCCTGAGCAGCGGACGTTCATGCTGGGAGGGCG |
| | | GCGGGTTGGAAGCAGGTGCCACCATGGCTAGTGGCAGCTGTCAG |
| | | GGGTGCGAAGAGGACGAGGAAACTCTGAAGAAGTTGATAGTCAG |
| | | GCTGAACAATGTCCAGGAAGGAAAACAGATAGAAACGCTGGTCC |
| | | AAATCCTGGAGGATCTGCTGGTGTTCACGTACTCCGAGCGCGCCT |
| | | CCAAGTTATTTCAAGGCAAAAATATCCATGTGCCTCTGTTGATCG |
| | | TCTTGGACTCCTATATGAGAGTCGCGAGTGTGCAGCAGGTGGGTT |
| | | GGTCACTTCTGTGCAAATTAATAGAAGTCTGTCCAGGTACAATGC |
| | | AAAGCTTAATGGGACCCCAGGATGTTGGAAATGATTGGGAAGTC |
| | | CTTGGTGTTCACCAATTGATTCTTAAAATGCTAACAGTTCATAAT |
| | | GCCAGTGTAAACTTGTCAGTGATTGGACTGAAGACCTTAGATCTC |
| | | CTCCTAACTTCAGGTAAAATCACCTTGCTGATATTGGATGAAGAA |
| | | AGTGATATTTTCATGTTAATTTTTGATGCCATGCACTCATTTCCAG |
| | | CCAATGATGAAGTCCAGAAACTTGGATGCAAAGCTTTACATGTG |
| | | CTGTTTGAGAGAGTCTCAGAGGAGCAACTGACTGAATTTGTTGAG |
| | | AACAAAGATTATATGATATTGTTAAGTGCGTTAACAAATTTTAAA |
| | | GATGAAGAGGAAATTGTGCTTCATGTGCTGCATTGTTTACATTCC |
| | | CTAGCGATTCCTTGCAATAATGTGGAAGTCCTCATGAGTGGCAAT |
| | | GTCAGGTGTTATAATATTGTGGTGGAAGCTATGAAAGCATTCCCT |
| | | ATGAGTGAAAGAATTCAAGAAGTGAGTTGCTGTTTGCTCCATAG |
| | | GCTTACATTAGGTAATTTTTTCAATATCCTGGTATTAAACGAAGT |
| | | CCATGAGTTTGTGGTGAAAGCTGTGCAGCAGTACCCAGAGAATG |
| | | CAGCATTGCAGATCTCAGCGCTCAGCTGTTTGGCCCTCCTCACTG |
| | | AGACTATTTTCTTAAATCAAGATTTAGAGGAAAAGAATGAGAAT |
| | | CAAGAGAATGATGATGAGGGGGAAGAAGATAAATTGTTTTGGCT |
| | | GGAAGCCTGTTACAAAGCATTAACGTGGCATAGAAAGAACAAGC |
| | | ACGTGCAGGAGGCCGCATGCTGGGCACTAAATAATCTCCTTATGT |
| | | ACCAAAACAGTTTACATGAGAAGATTGGAGATGAAGATGGCCAT |
| | | TTCCCAGCTCATAGGGAAGTGATGCTCTCCATGCTGATGCATTCT |
| | | TCATCAAAGGAAGTTTTCCAGGCATCTGCGAATGCATTGTCAACT |
| | | CTCTTAGAACAAAATGTTAATTTCAGAAAAATACTGTTATCAAAA |
| | | GGAATACACCTGAATGTTTTGGAGTTAATGCAGAAGCATATACAT |
| | | TCTCCTGAAGTGGCTGAAAGTGGCTGTAAAATGCTAAATCATCTT |
| | | TTTGAAGGAAGCAACACTTCCCTGGATATAATGGCAGCAGTGGT |
| | | CCCCAAAATACTAACAGTTATGAAACGTCATGAGACATCATTACC |
| | | AGTGCAGCTGGAGGCGCTTCGAGCTATTTTACATTTTATAGTGCC |
| | | TGGCATGCCAGAAGAATCCAGGGAGGATACAGAATTTCATCATA |
| | | AGCTAAATATGGTTAAAAAACAGTGTTTCAAGAATGATATTCAC |
| | | AAACTGGTCCTAGCAGCTTTGAACAGGTTCATTGGAAATCCTGGG |
| | | ATTCAGAAATGTGGATTAAAAGTAATTTCTTCTATTGTACATTTTC |
| | | CTGATGCATTAGAGATGTTATCCCTGGAAGGTGCTATGGATTCAG |
| | | TGCTTCACACACTGCAGATGTATCCAGATGACCAAGAAATTCAGT |
| | | GTCTGGGTTTAAGTCTTATAGGATACTTGATTACAAAGAAGAATG |
| | | TGTTCATAGGAACTGGACATCTGCTGGCAAAAATTCTGGTTTCCA |
| | | GCTTATACCGATTTAAGGATGTTGCTGAAATACAGACTAAAGGAT |
| | | TTCAGACAATCTTAGCAATCCTCAAATTGTCAGCATCTTTTTCTAA |
| | | GCTGCTGGTGCATCATTCATTTGACTTAGTAATATTCCATCAAAT |
| | | GTCTTCCAATATCATGGAACAAAAGGATCAACAGTTTCTAAACCT |
| | | CTGTTGCAAGTGTTTTGCAAAAGTAGCTATGGATGATTACTTAAA |
| | | AAATGTGATGCTAGAGAGAGCGTGTGATCAGAATAACAGCATCA |
| | | TGGTTGAATGCTTGCTTCTATTGGGAGCAGATGCCAATCAAGCAA |
| | | AGGAGGGATCTTCTTTAATTTGTCAGGTATGTGAGAAAGAGAGC |
| | | AGTCCCAAATTGGTGGAACTCTTACTGAATAGTGGATCTCGTGAA |
| | | CAAGATGTACGAAAAGCGTTGACGATAAGCATTGGGAAAGGTGA |
| | | CAGCCAGATCATCAGCTTGCTCTTAAGGAGGCTGGCCCTGGATGT |
| | | GGCCAACAATAGCATTTGCCTTGGAGGATTTTGTATAGGAAAAGT |
| | | TGAACCTTCTTGGCTTGGTCCTTTATTTCCAGATAAGACTTCTAAT |
| | | TTAAGGAAACAAACAAATATAGCATCTACACTAGCAAGAATGGT |
| | | GATCAGATATCAGATGAAAAGTGCTGTGGAAGAAGGAACAGCCT |
| | | CAGGCAGCGATGGAAATTTTTCTGAAGATGTGCTGTCTAAATTTG |
| | | ATGAATGGACCTTTATTCCTGACTCTTCTATGGACAGTGTGTTTGC |
| | | TCAAAGTGATGACCTGGATAGTGAAGGAAGTGAAGGCTCATTTC |
| | | TTGTGAAAAAGAAATCTAATTCAATTAGTGTAGGAGAATTTTACC |
| | | GAGATGCCGTATTACAGCGTTGCTCACCAAATTTGCAAAGACATT |
| | | CCAATTCCTTGGGGCCCATTTTTGATCATGAAGATTTACTGAAGC |
| | | GAAAAAGAAAAATATTATCTTCAGATGATTCACTCAGGTCATCA |
| | | AAACTTCAATCCCATATGAGGCATTCAGACAGCATTTCTTCTCTG |
| | | GCTTCTGAGAGAGAATATATTACATCACTAGACCTTTCAGCAAAT |
| | | GAACTAAGAGATATTGATGCCCTAAGCCAGAAATGCTGTATAAG |
| | | TGTTCATTTGGAGCATCTTGAAAAGCTGGAGCTTCACCAGAATGC |
| | | ACTCACGAGCTTTCCACAACAGCTATGTGAAACTCTGAAGAGTTT |
| | | GACACATTTGGACTTGCACAGTAATAAATTTACATCATTTCCTTC |
| | | TTATTTGTTGAAAATGAGTTGTATTGCTAATCTTGATGTCTCTCGA |
| | | AATGACATTGGACCCTCAGTGGTTTTAGATCCTACAGTGAAATGT |
| | | CCAACTCTGAAACAGTTTAACCTGTCATATAACCAGCTGTCTTTT |
| | | GTACCTGAGAACCTCACTGATGTGGTAGAGAAACTGGAGCAGCT |
| | | CATTTTAGAAGGAAATAAAATATCAGGGATATGCTCCCCCTTGAG |
| | | ACTGAAGGAACTGAAGATTTTAAACCTTAGTAAGAACCACATTTC |
| | | ATCCCTATCAGAGAACTTTCTTGAGGCTTGTCCTAAAGTGGAGAG |
| | | TTTCAGTGCCAGAATGAATTTTCTTGCTGCTATGCCTTTCTTGCCT |
| | | CCTTCTATGACAATCCTAAAATTATCTCAGAACAAATTTTCCTGT |
| | | ATTCCAGAAGCAATTTTAAATCTTCCACACTTGCGGTCTTTAGAT |
| | | ATGAGCAGCAATGATATTCAGTACCTACCAGGTCCCGCACACTG |
| | | GAAATCTTTGAACTTAAGGGAACTCTTATTTAGCCATAATCAGAT |
| | | CAGCATCTTGGACTTGAGTGAAAAAGCATATTTATGGTCTAGAGT |
| | | AGAGAAACTGCATCTTTCTCACAATAAACTGAAAGAGGAGCAGA |
| | | ATGAAGTCACAGATAATGGATAACCAAATCCATTTTGTGATTCCT |
| | | CCTGAGATTGGCTGTCTTGAAAATCTGACATCTCTGGATGTCAGT |
| | | TACAACTTGGAACTAA |

In some aspects, a region from an RNA sequence encoding a LRRK2 polypeptide sequence is targeted by an engineered polynucleotide as disclosed herein. Exemplary LRRK2 polypeptide sequences encoded by the isoforms previously provided are shown in Table 2. Any nucleotide of a polynucleotide sequence encoding any isoform from Table 2 can be targeted by an engineered polynucleotide as disclosed herein. In some cases, the nucleotides encoding any one of the 2,521 residues of a sequence associated with isoform 1 may be targeted utilizing the compositions and method provided herein. In some cases, a target nucleotide may encode an amino acid residue located among nucleotide residues 1-100, 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, 1901-2000, 2001-2100, 2101-2200, 2201-2300, 2301-2400, 2401-2500, 2501-2521 of isoform 1.

TABLE 2
Human LRRK2 Polypeptide Sequences associated with
isoforms provided in Table 1

| SEQ | | |
|---|---|---|
| ID NO: | Isoform | Polypeptide Sequence |
| 15 | Isoform 1 | MASGSCQGCEEDEETLKKLIVRLNNVQEGKQIETLVQILEDLLVFT |
| | | YSERASKLFQGKNIHVPLLIVLDSYMRVASVQQVGWSLLCKLIEVC |
| | | PGTMQSLMGPQDVGNDWEVLGVHQLILKMLTVHNASVNLSVIGLKT |
| | | LDLLLTSGKITLLILDEESDIFMLIFDAMHSFPANDEVQKLGCKAL |
| | | HVLFERVSEEQLTEFVENKDYMILLSALTNFKDEEEIVLHVLHCLH |
| | | SLAIPCNNVEVLMSGNVRCYNIVVEAMKAFPMSERIQEVSCCLLHR |
| | | LTLGNFFNILVLNEVHEFVVKAVQQYPENAALQISALSCLALLTET |
| | | IFLNQDLEEKNENQENDDEGEEDKLFWLEACYKALTWHRKNKHVQE |
| | | AACWALNNLLMYQNSLHEKIGDEDGHFPAHREVMLSMLMHSSSKEV |
| | | FQASANALSTLLEQNVNFRKILLSKGIHLNVLELMQKHIHSPEVAE |
| | | SGCKMLNHLFEGSNTSLDIMAAVVPKILTVMKRHETSLPVQLEALR |
| | | AILHFIVPGMPEESREDTEFHHKLNMVKKQCFKNDIHKLVLAALNR |
| | | FIGNPGIQKCGLKVISSIVHFPDALEMLSLEGAMDSVLHTLQMYPD |
| | | DQEIQCLGLSLIGYLITKKNVFIGTGHLLAKILVSSLYRFKDVAEI |
| | | QTKGFQTILAILKLSASFSKLLVHHSFDLVIFHQMSSNIMEQKDQQ |
| | | FLNLCCKCFAKVAMDDYLKNVMLERACDQNNSIMVECLLLLGADAN |
| | | QAKEGSSLICQVCEKESSPKLVELLLNSGSREQDVRKALTISIGKG |
| | | DSQIISLLLRRLALDVANNSICLGGFCIGKVEPSWLGPLFPDKTSN |
| | | LRKQTNIASTLARMVIRYQMKSAVEEGTASGSDGNFSEDVLSKFDE |
| | | WTFIPDSSMDSVFAQSDDLDSEGSEGSFLVKKKSNSISVGEFYRDA |
| | | VLQRCSPNLQRHSNSLGPIFDHEDLLKRKRKILSSDDSLRSSKLQS |
| | | HMRHSDSISSLASEREYITSLDLSANELRDIDALSQKCCISVHLEH |
| | | LEKLELHQNALTSFPQQLCETLKSLTHLDLHSNKFTSFPSYLLKMS |
| | | CIANLDVSRNDIGPSVVLDPTVKCPTLKQFNLSYNQLSFVPENLTD |
| | | VVEKLEQLILEGNKISGICSPLRLKELKILNLSKNHISSLSENFLE |
| | | ACPKVESFSARMNFLAAMPFLPPSMTILKLSQNKFSCIPEAILNLP |
| | | HLRSLDMSSNDIQYLPGPAHWKSLNLRELLFSHNQISILDLSEKAY |
| | | LWSRVEKLHLSHNKLKEIPPEIGCLENLTSLDVSYNLELRSFPNEM |
| | | GKLSKIWDLPLDELHLNFDFKHIGCKAKDIIRFLQQRLKKAVPYNR |
| | | MKLMIVGNTGSGKTTLLQQLMKTKKSDLGMQSATVGIDVKDWPIQI |
| | | RDKRKRDLVLNVWDFAGREEFYSTHPHFMTQRALYLAVYDLSKGQA |
| | | EVDAMKPWLFNIKARASSSPVILVGTHLDVSDEKQRKACMSKITKE |
| | | LLNKRGFPAIRDYHFVNATEESDALAKLRKTIINESLNFKIRDQLV |
| | | VGQLIPDCYVELEKIILSERKNVPIEFPVIDRKRLLQLVRENQLQL |
| | | DENELPHAVHFLNESGVLLHFQDPALQLSDLYFVEPKWLCKIMAQI |
| | | LTVKVEGCPKHPKGIISRRDVEKFLSKKRKFPKNYMSQYFKLLEKF |
| | | QIALPIGEEYLLVPSSLSDHRPVIELPHCENSEIIIRLYEMPYFPM |
| | | GFWSRLINRLLEISPYMLSGRERALRPNRMYWRQGIYLNWSPEAYC |
| | | LVGSEVLDNHPESFLKITVPSCRKGCILLGQVVDHIDSLMEEWFPG |
| | | LLEIDICGEGETLLKKWALYSFNDGEEHQKILLDDLMKKAEEGDLL |
| | | VNPDQPRLTIPISQIAPDLILADLPRNIMLNNDELEFEQAPEFLLG |
| | | DGSFGSVYRAAYEGEEVAVKIFNKHTSLRLLRQELVVLCHLHHPSL |
| | | ISLLAAGIRPRMLVMELASKGSLDRLLQQDKASLTRTLQHRIALHV |
| | | ADGLRYLHSAMIIYRDLKPHNVLLFTLYPNAAIIAKIADYGIAQYC |
| | | CRMGIKTSEGTPGFRAPEVARGNVIYNQQADVYSFGLLLYDILTTG |
| | | GRIVEGLKFPNEFDELEIQGKLPDPVKEYGCAPWPMVEKLIKQCLK |
| | | ENPQERPTSAQVFDILNSAELVCLTRRILLPKNVIVECMVATHHNS |
| | | RNASIWLGCGHTDRGQLSFLDLNTEGYTSEEVADSRILCLALVHLP |
| | | VEKESWIVSGTQSGTLLVINTEDGKKRHTLEKMTDSVTCLYCNSFS |
| | | KQSKQKNFLLVGTADGKLAIFEDKTVKLKGAAPLKILNIGNVSTPL |
| | | MCLSESTNSTERNVMWGGCGTKIFSFSNDFTIQKLIETRTSQLFSY |
| | | AAFSDSNIITVVVDTALYIAKQNSPVVEVWDKKTEKLCGLIDCVHF |
| | | LREVMVKENKESKHKMSYSGRVKTLCLQKNTALWIGTGGGHILLLD |
| | | LSTRRLIRVIYNFCNSVRVMMTAQLGSLKNVMLVLGYNRKNTEGTQ |
| | | KQKEIQSCLTVWDINLPHEVQNLEKHIEVRKELAEKMRRTSVE |
| 16 | X1 | MASGSCQGCEEDEETLKKLIVRLNNVQEGKQIETLVQILEDLLVFT |
| | | YSERASKLFQGKNIHVPLLIVLDSYMRVASVQQVGWSLLCKLIEVC |
| | | PGTMQSLMGPQDVGNDWEVLGVHQLILKMLTVHNASVNLSVIGLKT |
| | | LDLLLTSGKITLLILDEESDIFMLIFDAMHSFPANDEVQKLGCKAL |
| | | HVLFERVSEEQLTEFVENKDYMILLSALTNFKDEEEIVLHVLHCLH |
| | | SLAIPCNNVEVLMSGNVRCYNIVVEAMKAFPMSERIQEVSCCLLHR |
| | | LTLGNFFNILVLNEVHEFVVKAVQQYPENAALQISALSCLALLTET |
| | | IFLNQDLEEKNENQENDDEGEEDKLFWLEACYKALTWHRKNKHVQE |
| | | AACWALNNLLMYQNSLHEKIGDEDGHFPAHREVMLSMLMHSSSKEV |
| | | FQASANALSTLLEQNVNFRKILLSKGIHLNVLELMQKHIHSPEVAE |
| | | SGCKMLNHLFEGSNTSLDIMAAVVPKILTVMKRHETSLPVQLEALR |
| | | AILHFIVPGMPEESREDTEFHHKLNMVKKQCFKNDIHKLVLAALNR |
| | | FIGNPGIQKCGLKVISSIVHFPDALEMLSLEGAMDSVLHTLQMYPD |
| | | DQEIQCLGLSLIGYLITKKNVFIGTGHLLAKILVSSLYRFKDVAEI |
| | | QTKGFQTILAILKLSASFSKLLVHHSFDLVIFHQMSSNIMEQKDQQ |
| | | FLNLCCKCFAKVAMDDYLKNVMLERACDQNNSIMVECLLLLGADAN |
| | | QAKEGSSLICQVCEKESSPKLVELLLNSGSREQDVRKALTISIGKG |
| | | DSQIISLLLRRLALDVANNSICLGGFCIGKVEPSWLGPLFPDKTSN |
| | | LRKQTNIASTLARMVIRYQMKSAVEEGTASGSDGNFSEDVLSKFDE |
| | | WTFIPDSSMDSVFAQSDDLDSEGSEGSFLVKKKSNSISVGEFYRDA |
| | | VLQRCSPNLQRHSNSLGPIFDHEDLLKRKRKILSSDDSLRSSKLQS |
| | | HMRHSDSISSLASEREYITSLDLSANELRDIDALSQKCCISVHLEH |
| | | LEKLELHQNALTSFPQQLCETLKSLTHLDLHSNKFTSFPSYLLKMS |
| | | CIANLDVSRNDIGPSVVLDPTVKCPTLKQFNLSYNQLSFVPENLTD |
| | | VVEKLEQLILEGNKISGICSPLRLKELKILNLSKNHISSLSENFLE |
| | | ACPKVESFSARMNFLAAMPFLPPSMTILKLSQNKFSCIPEAILNLP |
| | | HLRSLDMSSNDIQYLPGPAHWKSLNLRELLFSHNQISILDLSEKAY |
| | | LWSRVEKLHLSHNKLKEIPPEIGCLENLTSLDVSYNLELRSFPNEM |
| | | GKLSKIWDLPLDELHLNFDFKHIGCKAKDIIRFLQQRLKKAVPYNR |
| | | MKLMIVGNTGSGKTTLLQQLMKTKKSDLGMQSATVGIDVKDWPIQI |
| | | RDKRKRDLVLNVWDFAGREEFYSTHPHFMTQRALYLAVYDLSKGQA |
| | | EVDAMKPWLFNIKARASSSPVILVGTHLDVSDEKQRKACMSKITKE |
| | | LLNKRGFPAIRDYHFVNATEESDALAKLRKTIINESLNFKIRDQLV |
| | | VGQLIPDCYVELEKIILSERKNVPIEFPVIDRKRLLQLVRENQLQL |
| | | DENELPHAVHFLNESGVLLHFQDPALQLSDLYFVEPKWLCKIMAQI |
| | | LTVKVEGCPKHPKGIISRRDVEKFLSKKRKFPKNYMSQYFKLLEKF |
| | | QIALPIGEEYLLVPSSLSDHRPVIELPHCENSEIIIRLYEMPYFPM |
| | | GFWSRLINRLLEISPYMLSGRERALRPNRMYWRQGIYLNWSPEAYC |
| | | LVGSEVLDNHPESFLKITVPSCRKGCILLGQVVDHIDSLMEEWFPG |
| | | LLEIDICGEGETLLKKWALYSFNDGEEHQKILLDDLMKKAEEGDLL |
| | | VNPDQPRLTIPISQIAPDLILADLPRNIMLNNDELEFEQAPEFLLG |
| | | DGSFGSVYRAAYEGEEVAVKIFNKHTSLRLLRQELVVLCHLHHPSL |
| | | ISLLAAGIRPRMLVMELASKGSLDRLLQQDKASLTRTLQHRIALHV |
| | | ADGLRYLHSAMIIYRDLKPHNVLLFTLYPNAAIIAKIADYGIAQYC |
| | | CRMGIKTSEGTPGFRAPEVARGNVIYNQQADVYSFGLLLYDILTTG |
| | | GRIVEGLKFPNEFDELEIQGKLPDPVKEYGCAPWPMVEKLIKQCLK |
| | | ENPQERPTSAQVFDILNSAELVCLTRRILLPKNVIVECMVATHHNS |
| | | RNASIWLGCGHTDRGQLSFLDLNTEGYTSEEVADSRILCLALVHLP |
| | | VEKESWIVSGTQSGTLLVINTEDGKKRHTLEKMTDSVTCLYCNSFS |
| | | KQSKQKNFLLVGTADGKLAIFEDKTVKLKGAAPLKILNIGNVSTPL |
| | | MCLSESTNSTERNVMWGGCGTKIFSFSNDFTIQKLIETRTSQLFSY |
| | | AAFSDSNIITVVVDTALYIAKQNSPVVEVWDKKTEKLCGLIDCVHF |
| | | LREVMVKENKESKHKMSYSGRVKTLCLQKNTALWIGTGGGHILLLD |
| | | LSTRRLIRVIYNFCNSVRVMMTAQLGSLKNVMLVLGYNRKNTEEIQ |
| | | SCLTVWDINLPHEVQNLEKHIEVRKELAEKMRRTSVE |
| 17 | X2 | MASGSCQGCEEDEETLKKLIVRLNNVQEGKQIETLVQILEDLLVFT |
| | | YSERASKLFQGKNIHVPLLIVLDSYMRVASVQQVGWSLLCKLIEVC |
| | | PGTMQSLMGPQDVGNDWEVLGVHQLILKMLTVHNASVNLSVIGLKT |
| | | LDLLLTSGKITLLILDEESDIFMLIFDAMHSFPANDEVQKLGCKAL |
| | | HVLFERVSEEQLTEFVENKDYMILLSALTNFKDEEEIVLHVLHCLH |
| | | SLAIPCNNVEVLMSGNVRCYNIVVEAMKAFPMSERIQEVSCCLLHR |
| | | LTLGNFFNILVLNEVHEFVVKAVQQYPENAALQISALSCLALLTET |
| | | IFLNQDLEEKNENQENDDEGEEDKLFWLEACYKALTWHRKNKHVQE |
| | | AACWALNNLLMYQNSLHEKIGDEDGHFPAHREVMLSMLMHSSSKEV |
| | | FQASANALSTLLEQNVNFRKILLSKGIHLNVLELMQKHIHSPEVAE |
| | | SGCKMLNHLFEGSNTSLDIMAAVVPKILTVMKRHETSLPVQLEALR |
| | | AILHFIVPGMPEESREDTEFHHKLNMVKKQCFKNDIHKLVLAALNR |
| | | FIGNPGIQKCGLKVISSIVHFPDALEMLSLEGAMDSVLHTLQMYPD |
| | | DQEIQCLGLSLIGYLITKKNVFIGTGHLLAKILVSSLYRFKDVAEI |
| | | QTKGFQTILAILKLSASFSKLLVHHSFDLVIFHQMSSNIMEQKDQQ |
| | | FLNLCCKCFAKVAMDDYLKNVMLERACDQNNSIMVECLLLLGADAN |
| | | QAKEGSSLICQVCEKESSPKLVELLLNSGSREQDVRKALTISIGKG |
| | | DSQIISLLLRRLALDVANNSICLGGFCIGKVEPSWLGPLFPDKTSN |
| | | LRKQTNIASTLARMVIRYQMKSAVEEGTASGSDGNFSEDVLSKFDE |
| | | WTFIPDSSMDSVFAQSDDLDSEGSEGSFLVKKKSNSISVGEFYRDA |
| | | VLQRCSPNLQRHSNSLGPIFDHEDLLKRKRKILSSDDSLRSSKLQS |
| | | HMRHSDSISSLASEREYITSLDLSANELRDIDALSQKCCISVHLEH |
| | | LEKLELHQNALTSFPQQLCETLKSLTHLDLHSNKFTSFPSYLLKMS |
| | | CIANLDVSRNDIGPSVVLDPTVKCPTLKQFNLSYNQLSFVPENLTD |
| | | VVEKLEQLILEGNKISGICSPLRLKELKILNLSKNHISSLSENFLE |
| | | ACPKVESFSARMNFLAAMPFLPPSMTILKLSQNKFSCIPEAILNLP |
| | | HLRSLDMSSNDIQYLPGPAHWKSLNLRELLFSHNQISILDLSEKAY |
| | | LWSRVEKLHLSHNKLKEIPPEIGCLENLTSLDVSYNLELRSFPNEM |
| | | GKLSKIWDLPLDELHLNFDFKHIGCKAKDIIRFLQQRLKKAVPYNR |
| | | MKLMIVGNTGSGKTTLLQQLMKTKKSDLGMQSATVGIDVKDWPIQI |
| | | RDKRKRDLVLNVWDFAGREEFYSTHPHFMTQRALYLAVYDLSKGQA |
| | | EVDAMKPWLFNIKARASSSPVILVGTHLDVSDEKQRKACMSKITKE |
| | | LLNKRGFPAIRDYHFVNATEESDALAKLRKTIINESLNFKIRDQLV |
| | | VGQLIPDCYVELEKIILSERKNVPIEFPVIDRKRLLQLVRENQLQL |
| | | DENELPHAVHFLNESGVLLHFQDPALQLSDLYFVEPKWLCKIMAQI |
| | | LTVKVEGCPKHPKGIISRRDVEKFLSKKRKFPKNYMSQYFKLLEKF |
| | | QIALPIGEEYLLVPSSLSDHRPVIELPHCENSEIIIRLYEMPYFPM |
| | | GFWSRLINRLLEISPYMLSGRERALRPNRMYWRQGIYLNWSPEAYC |
| | | LVGSEVLDNHPESFLKITVPSCRKGCILLGQVVDHIDSLMEEWFPG |
| | | LLEIDICGEGETLLKKWALYSFNDGEEHQKILLDDLMKKAEEGDLL |
| | | VNPDQPRLTIPISQIAPDLILADLPRNIMLNNDELEFEQAPEFLLG |
| | | DGSFGSVYRAAYEGEEVAVKIFNKHTSLRLLRQELVVLCHLHHPSL |
| | | ISLLAAGIRPRMLVMELASKGSLDRLLQQDKASLTRTLQHRIALHV |
| | | ADGLRYLHSAMIIYRDLKPHNVLLFTLYPNAAIIAKIADYGIAQYC |
| | | CRMGIKTSEGTPGFRAPEVARGNVIYNQQADVYSFGLLLYDILTTG |
| | | GRIVEGLKFPNEFDELEIQGKLPDPVKEYGCAPWPMVEKLIKQCLK |
| | | ENPQERPTSAQVFDILNSAELVCLTRRILLPKNVIVECMVATHHNS |
| | | RNASIWLGCGHTDRGQLSFLDLNTEGYTSEEVADSRILCLALVHLP |
| | | VEKESWIVSGTQSGTLLVINTEDGKKRHTLEKMTDSVTCLYCNSFS |
| | | KQSKQKNFLLVGTADGKLAIFEDKTVKLKGAAPLKILNIGNVSTPL |
| | | MCLSESTNSTERNVMWGGCGTKIFSFSNDFTIQKLIETRTSQLFSY |
| | | AAFSDSNIITVVVDTALYIAKQNSPVVEVWDKKTEKLCGLIDCVHF |
| | | LREVMVKENKESKHKMSYSGRVKTLCLQKNTALWIGTGGGHILLLD |
| | | LSTRRLIRVIYNFCNSVRVMMTAQLEIQSCLTVWDINLPHEVQNLE |
| | | KHIEVRKELAEKMRRTSVE |
| 18 | X3 | MLSMLMHSSSKEVFQASANALSTLLEQNVNFRKILLSKGIHLNVLE |
| | | LMQKHIHSPEVAESGCKMLNHLFEGSNTSLDIMAAVVPKILTVMKR |
| | | HETSLPVQLEALRAILHFIVPGMPEESREDTEFHHKLNMVKKQCFK |
| | | NDIHKLVLAALNRFIGNPGIQKCGLKVISSIVHFPDALEMLSLEGA |
| | | MDSVLHTLQMYPDDQEIQCLGLSLIGYLITKKNVFIGTGHLLAKIL |
| | | VSSLYRFKDVAEIQTKGFQTILAILKLSASFSKLLVHHSFDLVIFH |
| | | QMSSNIMEQKDQQFLNLCCKCFAKVAMDDYLKNVMLERACDQNNSI |
| | | MVECLLLLGADANQAKEGSSLICQVCEKESSPKLVELLLNSGSREQ |
| | | DVRKALTISIGKGDSQIISLLLRRLALDVANNSICLGGFCIGKVEP |
| | | SWLGPLFPDKTSNLRKQTNIASTLARMVIRYQMKSAVEEGTASGSD |
| | | GNFSEDVLSKFDEWTFIPDSSMDSVFAQSDDLDSEGSEGSFLVKKK |
| | | SNSISVGEFYRDAVLQRCSPNLQRHSNSLGPIFDHEDLLKRKRKIL |
| | | SSDDSLRSSKLQSHMRHSDSISSLASEREYITSLDLSANELRDIDA |
| | | LSQKCCISVHLEHLEKLELHQNALTSFPQQLCETLKSLTHLDLHSN |
| | | KFTSFPSYLLKMSCIANLDVSRNDIGPSVVLDPTVKCPTLKQFNLS |
| | | YNQLSFVPENLTDVVEKLEQLILEGNKISGICSPLRLKELKILNLS |
| | | KNHISSLSENFLEACPKVESFSARMNFLAAMPFLPPSMTILKLSQN |
| | | KFSCIPEAILNLPHLRSLDMSSNDIQYLPGPAHWKSLNLRELLFSH |
| | | NQISILDLSEKAYLWSRVEKLHLSHNKLKEIPPEIGCLENLTSLDV |
| | | SYNLELRSFPNEMGKLSKIWDLPLDELHLNFDFKHIGCKAKDIIRF |
| | | LQQRLKKAVPYNRMKLMIVGNTGSGKTTLLQQLMKTKKSDLGMQSA |
| | | TVGIDVKDWPIQIRDKRKRDLVLNVWDFAGREEFYSTHPHFMTQRA |
| | | LYLAVYDLSKGQAEVDAMKPWLFNIKARASSSPVILVGTHLDVSDE |
| | | KQRKACMSKITKELLNKRGFVELEKIILSERKNVPIEFPVIDRKRL |
| | | LQLVRENQLQLDENELPHAVHFLNESGVLLHFQDPALQLSDLYFVE |
| | | PKWLCKIMAQILTVKVEGCPKHPKGIISRRDVEKFLSKKRKFPKNY |
| | | MSQYFKLLEKFQIALPIGEEYLLVPSSLSDHRPVIELPHCENSEII |
| | | IRLYEMPYFPMGFWSRLINRLLEISPYMLSGRERALRPNRMYWRQG |
| | | IYLNWSPEAYCLVGSEVLDNHPESFLKITVPSCRKGCILLGQVVDH |
| | | IDSLMEEWFPGLLEIDICGEGETLLKKWALYSFNDGEEHQKILLDD |
| | | LMKKAEEGDLLVNPDQPRLTIPISQIAPDLILADLPRNIMLNNDEL |
| | | EFEQAPEFLLGDGSFGSVYRAAYEGEEVAVKIFNKHTSLRLLRQEL |
| | | VVLCHLHHPSLISLLAAGIRPRMLVMELASKGSLDRLLQQDKASLT |
| | | RTLQHRIALHVADGLRYLHSAMIIYRDLKPHNVLLFTLYPNAAIIA |
| | | KIADYGIAQYCCRMGIKTSEGTPGFRAPEVARGNVIYNQQADVYSF |
| | | GLLLYDILTTGGRIVEGLKFPNEFDELEIQGKLPDPVKEYGCAPWP |
| | | MVEKLIKQCLKENPQERPTSAQVFDILNSAELVCLTRRILLPKNVI |
| | | VECMVATHHNSRNASIWLGCGHTDRGQLSFLDLNTEGYTSEEVADS |
| | | RILCLALVHLPVEKESWIVSGTQSGTLLVINTEDGKKRHTLEKMTD |
| | | SVTCLYCNSFSKQSKQKNFLLVGTADGKLAIFEDKTVKLKGAAPLK |
| | | ILNIGNVSTPLMCLSESTNSTERNVMWGGCGTKIFSFSNDFTIQKL |
| | | IETRTSQLFSYAAFSDSNIITVVVDTALYIAKQNSPVVEVWDKKTE |
| | | KLCGLIDCVHFLREVMVKENKESKHKMSYSGRVKTLCLQKNTALWI |
| | | GTGGGHILLLDLSTRRLIRVIYNFCNSVRVMMTAQLGSLKNVMLVL |
| | | GYNRKNTEGTQKQKEIQSCLTVWDINLPHEVQNLEKHIEVRKELAE |
| | | KMRRTSVE |
| 19 | X4 | MASGSCQGCEEDEETLKKLIVRLNNVQEGKQIETLVQILEDLLVFT |
| | | YSERASKLFQGKNIHVPLLIVLDSYMRVASVQQVGWSLLCKLIEVC |
| | | PGTMQSLMGPQDVGNDWEVLGVHQLILKMLTVHNASVNLSVIGLKT |
| | | LDLLLTSGKITLLILDEESDIFMLIFDAMHSFPANDEVQKLGCKAL |
| | | HVLFERVSEEQLTEFVENKDYMILLSALTNFKDEEEIVLHVLHCLH |
| | | SLAIPCNNVEVLMSGNVRCYNIVVEAMKAFPMSERIQEVSCCLLHR |
| | | LTLGNFFNILVLNEVHEFVVKAVQQYPENAALQISALSCLALLTET |
| | | IFLNQDLEEKNENQENDDEGEEDKLFWLEACYKALTWHRKNKHVQE |
| | | AACWALNNLLMYQNSLHEKIGDEDGHFPAHREVMLSMLMHSSSKEV |
| | | FQASANALSTLLEQNVNFRKILLSKGIHLNVLELMQKHIHSPEVAE |
| | | SGCKMLNHLFEGSNTSLDIMAAVVPKILTVMKRHETSLPVQLEALR |
| | | AILHFIVPGMPEESREDTEFHHKLNMVKKQCFKNDIHKLVLAALNR |
| | | FIGNPGIQKCGLKVISSIVHFPDALEMLSLEGAMDSVLHTLQMYPD |
| | | DQEIQCLGLSLIGYLITKKNVFIGTGHLLAKILVSSLYRFKDVAEI |
| | | QTKGFQTILAILKLSASFSKLLVHHSFDLVIFHQMSSNIMEQKDQQ |
| | | FLNLCCKCFAKVAMDDYLKNVMLERACDQNNSIMVECLLLLGADAN |
| | | QAKEGSSLICQVCEKESSPKLVELLLNSGSREQDVRKALTISIGKG |
| | | DSQIISLLLRRLALDVANNSICLGGFCIGKVEPSWLGPLFPDKTSN |
| | | LRKQTNIASTLARMVIRYQMKSAVEEGTASGSDGNFSEDVLSKFDE |
| | | WTFIPDSSMDSVFAQSDDLDSEGSEGSFLVKKKSNSISVGEFYRDA |
| | | VLQRCSPNLQRHSNSLGPIFDHEDLLKRKRKILSSDDSLRSSKLQS |
| | | HMRHSDSISSLASEREYITSLDLSANELRDIDALSQKCCISVHLEH |
| | | LEKLELHQNALTSFPQQLCETLKSLTHLDLHSNKFTSFPSYLLKMS |
| | | CIANLDVSRNDIGPSVVLDPTVKCPTLKQFNLSYNQLSFVPENLTD |
| | | VVEKLEQLILEGNKISGICSPLRLKELKILNLSKNHISSLSENFLE |
| | | ACPKVESFSARMNFLAAMPFLPPSMTILKLSQNKFSCIPEAILNLP |
| | | HLRSLDMSSNDIQYLPGPAHWKSLNLRELLFSHNQISILDLSEKAY |
| | | LWSRVEKLHLSHNKLKEIPPEIGCLENLTSLDVSYNLELRSFPNEM |
| | | GKLSKIWDLPLDELHLNFDFKHIGCKAKDIIRFLQQRLKKAVPYNR |
| | | MKLMIVGNTGSGKTTLLQQLMKTKKSDLGMQSATVGIDVKDWPIQI |
| | | RDKRKRDLVLNVWDFAGREEFYSTHPHFMTQRALYLAVYDLSKGQA |
| | | EVDAMKPWLFNIKARASSSPVILVGTHLDVSDEKQRKACMSKITKE |
| | | LLNKRGFPAIRDYHFVNATEESDALAKLRKTIINESLNFKIRDQLV |
| | | VGQLIPDCYVELEKIILSERKNVPIEFPVIDRKRLLQLVRENQLQL |
| | | DENELPHAVHFLNESGVLLHFQDPALQLSDLYFVEPKWLCKIMAQF |
| | | V |
| 20 | X5 | MASGSCQGCEEDEETLKKLIVRLNNVQEGKQIETLVQILEDLLVFT |
| | | YSERASKLFQGKNIHVPLLIVLDSYMRVASVQQVGWSLLCKLIEVC |
| | | PGTMQSLMGPQDVGNDWEVLGVHQLILKMLTVHNASVNLSVIGLKT |
| | | LDLLLTSGKITLLILDEESDIFMLIFDAMHSFPANDEVQKLGCKAL |
| | | HVLFERVSEEQLTEFVENKDYMILLSALTNFKDEEEIVLHVLHCLH |
| | | SLAIPCNNVEVLMSGNVRCYNIVVEAMKAFPMSERIQEVSCCLLHR |
| | | LTLGNFFNILVLNEVHEFVVKAVQQYPENAALQISALSCLALLTET |
| | | IFLNQDLEEKNENQENDDEGEEDKLFWLEACYKALTWHRKNKHVQE |
| | | AACWALNNLLMYQNSLHEKIGDEDGHFPAHREVMLSMLMHSSSKEV |
| | | FQASANALSTLLEQNVNFRKILLSKGIHLNVLELMQKHIHSPEVAE |
| | | SGCKMLNHLFEGSNTSLDIMAAVVPKILTVMKRHETSLPVQLEALR |
| | | AILHFIVPGMPEESREDTEFHHKLNMVKKQCFKNDIHKLVLAALNR |
| | | FIGNPGIQKCGLKVISSIVHFPDALEMLSLEGAMDSVLHTLQMYPD |
| | | DQEIQCLGLSLIGYLITKKNVFIGTGHLLAKILVSSLYRFKDVAEI |
| | | QTKGFQTILAILKLSASFSKLLVHHSFDLVIFHQMSSNIMEQKDQQ |
| | | FLNLCCKCFAKVAMDDYLKNVMLERACDQNNSIMVECLLLLGADAN |
| | | QAKEGSSLICQVCEKESSPKLVELLLNSGSREQDVRKALTISIGKG |
| | | DSQIISLLLRRLALDVANNSICLGGFCIGKVEPSWLGPLFPDKTSN |
| | | LRKQTNIASTLARMVIRYQMKSAVEEGTASGSDGNFSEDVLSKFDE |
| | | WTFIPDSSMDSVFAQSDDLDSEGSEGSFLVKKKSNSISVGEFYRDA |
| | | VLQRCSPNLQRHSNSLGPIFDHEDLLKRKRKILSSDDSLRSSKLQS |
| | | HMRHSDSISSLASEREYITSLDLSANELRDIDALSQKCCISVHLEH |
| | | LEKLELHQNALTSFPQQLCETLKSLTHLDLHSNKFTSFPSYLLKMS |
| | | CIANLDVSRNDIGPSVVLDPTVKCPTLKQFNLSYNQLSFVPENLTD |
| | | VVEKLEQLILEGNKISGICSPLRLKELKILNLSKNHISSLSENFLE |
| | | ACPKVESFSARMNFLAAMPFLPPSMTILKLSQNKFSCIPEAILNLP |
| | | HLRSLDMSSNDIQYLPGPAHWKSLNLRELLFSHNQISILDLSEKAY |
| | | LWSRVEKLHLSHNKLKEIPPEIGCLENLTSLDVSYNLELRSFPNEM |
| | | GKLSKIWDLPLDELHLNFDFKHIGCKAKDIIRFLQQRLKKAVPYNR |
| | | MKLMIVGNTGSGKTTLLQQLMKTKKSDLGMQSATVGIDVKDWPIQI |
| | | RDKRKRDLVLNVWDFAGREEFYSTHPHFMTQRALYLAVYDLSKGQA |
| | | EVDAMKPWLFNIKARASSSPVILVGTHLDVSDEKQRKACMSKITKE |
| | | LLNKRGFPAIRDYHFVNATEESDALAKLRKTIINESLNFKESFFIF |
| | | KTQHCS |
| 21 | X6 | MIHSTLKSLTHLDLHSNKFTSFPSYLLKMSCIANLDVSRNDIGPSV |
| | | VLDPTVKCPTLKQFNLSYNQLSFVPENLTDVVEKLEQLILEGNKIS |
| | | GICSPLRLKELKILNLSKNHISSLSENFLEACPKVESFSARMNFLA |
| | | AMPFLPPSMTILKLSQNKFSCIPEAILNLPHLRSLDMSSNDIQYLP |
| | | GPAHWKSLNLRELLFSHNQISILDLSEKAYLWSRVEKLHLSHNKLK |
| | | EIPPEIGCLENLTSLDVSYNLELRSFPNEMGKLSKIWDLPLDELHL |
| | | NFDFKHIGCKAKDIIRFLQQRLKKAVPYNRMKLMIVGNTGSGKTTL |
| | | LQQLMKTKKSDLGMQSATVGIDVKDWPIQIRDKRKRDLVLNVWDFA |
| | | GREEFYSTHPHFMTQRALYLAVYDLSKGQAEVDAMKPWLFNIKARA |
| | | SSSPVILVGTHLDVSDEKQRKACMSKITKELLNKRGFPAIRDYHFV |
| | | NATEESDALAKLRKTIINESLNFKIRDQLVVGQLIPDCYVELEKII |
| | | LSERKNVPIEFPVIDRKRLLQLVRENQLQLDENELPHAVHFLNESG |
| | | VLLHFQDPALQLSDLYFVEPKWLCKIMAQILTVKVEGCPKHPKGII |
| | | SRRDVEKFLSKKRKFPKNYMSQYFKLLEKFQIALPIGEEYLLVPSS |
| | | LSDHRPVIELPHCENSEIIIRLYEMPYFPMGFWSRLINRLLEISPY |
| | | MLSGRERALRPNRMYWRQGIYLNWSPEAYCLVGSEVLDNHPESFLK |
| | | ITVPSCRKGCILLGQVVDHIDSLMEEWFPGLLEIDICGEGETLLKK |
| | | WALYSFNDGEEHQKILLDDLMKKAEEGDLLVNPDQPRLTIPISQIA |
| | | PDLILADLPRNIMLNNDELEFEQAPEFLLGDGSFGSVYRAAYEGEE |
| | | VAVKIFNKHTSLRLLRQELVVLCHLHHPSLISLLAAGIRPRMLVME |
| | | LASKGSLDRLLQQDKASLTRTLQHRIALHVADGLRYLHSAMIIYRD |
| | | LKPHNVLLFTLYPNAAIIAKIADYGIAQYCCRMGIKTSEGTPGFRA |
| | | PEVARGNVIYNQQADVYSFGLLLYDILTTGGRIVEGLKFPNEFDEL |
| | | EIQGKLPDPVKEYGCAPWPMVEKLIKQCLKENPQERPTSAQVFDIL |
| | | NSAELVCLTRRILLPKNVIVECMVATHHNSRNASIWLGCGHTDRGQ |
| | | LSFLDLNTEGYTSEEVADSRILCLALVHLPVEKESWIVSGTQSGTL |
| | | LVINTEDGKKRHTLEKMTDSVTCLYCNSFSKQSKQKNFLLVGTADG |
| | | KLAIFEDKTVKLKGAAPLKILNIGNVSTPLMCLSESTNSTERNVMW |
| | | GGCGTKIFSFSNDFTIQKLIETRTSQLFSYAAFSDSNIITVVVDTA |
| | | LYIAKQNSPVVEVWDKKTEKLCGLIDCVHFLREVMVKENKESKHKM |
| | | SYSGRVKTLCLQKNTALWIGTGGGHILLLDLSTRRLIRVIYNFCNS |
| | | VRVMMTAQLGSLKNVMLVLGYNRKNTEGTQKQKEIQSCLTVWDINL |
| | | PHEVQNLEKHIEVRKELAEKMRRTSVE |
| 22 | X7 | MKSQIMDNQIHFVIPPEIGCLENLTSLDVSYNLELRSFPNEMGKLS |
| | | KIWDLPLDELHLNFDFKHIGCKAKDIIRFLQQRLKKAVPYNRMKLM |
| | | IVGNTGSGKTTLLQQLMKTKKSDLGMQSATVGIDVKDWPIQIRDKR |
| | | KRDLVLNVWDFAGREEFYSTHPHFMTQRALYLAVYDLSKGQAEVDA |
| | | MKPWLFNIKARASSSPVILVGTHLDVSDEKQRKACMSKITKELLNK |
| | | RGFPAIRDYHFVNATEESDALAKLRKTIINESLNFKIRDQLVVGQL |
| | | IPDCYVELEKIILSERKNVPIEFPVIDRKRLLQLVRENQLQLDENE |
| | | LPHAVHFLNESGVLLHFQDPALQLSDLYFVEPKWLCKIMAQILTVK |
| | | VEGCPKHPKGIISRRDVEKFLSKKRKFPKNYMSQYFKLLEKFQIAL |
| | | PIGEEYLLVPSSLSDHRPVIELPHCENSEIIIRLYEMPYFPMGFWS |
| | | RLINRLLEISPYMLSGRERALRPNRMYWRQGIYLNWSPEAYCLVGS |
| | | EVLDNHPESFLKITVPSCRKGCILLGQVVDHIDSLMEEWFPGLLEI |
| | | DICGEGETLLKKWALYSFNDGEEHQKILLDDLMKKAEEGDLLVNPD |
| | | QPRLTIPISQIAPDLILADLPRNIMLNNDELEFEQAPEFLLGDGSF |
| | | GSVYRAAYEGEEVAVKIFNKHTSLRLLRQELVVLCHLHHPSLISLL |
| | | AAGIRPRMLVMELASKGSLDRLLQQDKASLTRTLQHRIALHVADGL |
| | | RYLHSAMIIYRDLKPHNVLLFTLYPNAAIIAKIADYGIAQYCCRMG |
| | | IKTSEGTPGFRAPEVARGNVIYNQQADVYSFGLLLYDILTTGGRIV |
| | | EGLKFPNEFDELEIQGKLPDPVKEYGCAPWPMVEKLIKQCLKENPQ |
| | | ERPTSAQVFDILNSAELVCLTRRILLPKNVIVECMVATHHNSRNAS |
| | | IWLGCGHTDRGQLSFLDLNTEGYTSEEVADSRILCLALVHLPVEKE |
| | | SWIVSGTQSGTLLVINTEDGKKRHTLEKMTDSVTCLYCNSFSKQSK |
| | | QKNFLLVGTADGKLAIFEDKTVKLKGAAPLKILNIGNVSTPLMCLS |
| | | ESTNSTERNVMWGGCGTKIFSFSNDFTIQKLIETRTSQLFSYAAFS |
| | | DSNIITWVDTALYIAKQNSPVVEVWDKKTEKLCGLIDCVHFLREVM |
| | | VKENKESKHKMSYSGRVKTLCLQKNTALWIGTGGGHILLLDLSTRR |
| | | LIRVIYNFCNSVRVMMTAQLGSLKNVMLVLGYNRKNTEGTQKQKEI |
| | | QSCLTVWDINLPHEVQNLEKHIEVRKELAEKMRRTSVE |
| 23 | X8 | MASGSCQGCEEDEETLKKLIVRLNNVQEGKQIETLVQILEDLLVFT |
| | | YSERASKLFQGKNIHVPLLIVLDSYMRVASVQQVGWSLLCKLIEVC |
| | | PGTMQSLMGPQDVGNDWEVLGVHQLILKMLTVHNASVNLSVIGLKT |
| | | LDLLLTSGKITLLILDEESDIFMLIFDAMHSFPANDEVQKLGCKAL |
| | | HVLFERVSEEQLTEFVENKDYMILLSALTNFKDEEEIVLHVLHCLH |
| | | SLAIPCNNVEVLMSGNVRCYNIVVEAMKAFPMSERIQEVSCCLLHR |
| | | LTLGNFFNILVLNEVHEFVVKAVQQYPENAALQISALSCLALLTET |
| | | IFLNQDLEEKNENQENDDEGEEDKLFWLEACYKALTWHRKNKHVQE |
| | | AACWALNNLLMYQNSLHEKIGDEDGHFPAHREVMLSMLMHSSSKEV |
| | | FQASANALSTLLEQNVNFRKILLSKGIHLNVLELMQKHIHSPEVAE |
| | | SGCKMLNHLFEGSNTSLDIMAAVVPKILTVMKRHETSLPVQLEALR |
| | | AILHFIVPGMPEESREDTEFHHKLNMVKKQCFKNDIHKLVLAALNR |
| | | FIGNPGIQKCGLKVISSIVHFPDALEMLSLEGAMDSVLHTLQMYPD |
| | | DQEIQCLGLSLIGYLITKKNVFIGTGHLLAKILVSSLYRFKDVAEI |
| | | QTKGFQTILAILKLSASFSKLLVHHSFDLVIFHQMSSNIMEQKDQQ |
| | | FLNLCCKCFAKVAMDDYLKNVMLERACDQNNSIMVECLLLLGADAN |
| | | QAKEGSSLICQVCEKESSPKLVELLLNSGSREQDVRKALTISIGKG |
| | | DSQIISLLLRRLALDVANNSICLGGFCIGKVEPSWLGPLFPDKTSN |
| | | LRKQTNIASTLARMVIRYQMKSAVEEGTASGSDGNFSEDVLSKFDE |
| | | WTFIPDSSMDSVFAQSDDLDSEGSEGSFLVKKKSNSISVGEFYRDA |
| | | VLQRCSPNLQRHSNSLGPIFDHEDLLKRKRKILSSDDSLRSSKLQS |
| | | HMRHSDSISSLASEREYITSLDLSANELRDIDALSQKCCISVHLEH |
| | | LEKLELHQNALTSFPQQLCETLKSLTHLDLHSNKFTSFPSYLLKMS |
| | | CIANLDVSRNDIGPSVVLDPTVKCPTLKQFNLSYNQLSFVPENLTD |
| | | VVEKLEQLILEGNKISGICSPLRLKELKILNLSKNHISSLSENFLE |
| | | ACPKVESFSARMNFLAAMPFLPPSMTILKLSQNKFSCIPEAILNLP |
| | | HLRSLDMSSNDIQYLPGPAHWKSLNLRELLFSHNQISILDLSEKAY |
| | | LWSRVEKLHLSHNKLKEVSSTAIKKGCAL |
| 24 | X9 | MASGSCQGCEEDEETLKKLIVRLNNVQEGKQIETLVQILEDLLVFT |
| | | YSERASKLFQGKNIHVPLLIVLDSYMRVASVQQVGWSLLCKLIEVC |
| | | PGTMQSLMGPQDVGNDWEVLGVHQLILKMLTVHNASVNLSVIGLKT |
| | | LDLLLTSGKITLLILDEESDIFMLIFDAMHSFPANDEVQKLGCKAL |
| | | HVLFERVSEEQLTEFVENKDYMILLSALTNFKDEEEIVLHVLHCLH |
| | | SLAIPCNNVEVLMSGNVRCYNIVVEAMKAFPMSERIQEVSCCLLHR |
| | | LTLGNFFNILVLNEVHEFVVKAVQQYPENAALQISALSCLALLTET |
| | | IFLNQDLEEKNENQENDDEGEEDKLFWLEACYKALTWHRKNKHVQE |
| | | AACWALNNLLMYQNSLHEKIGDEDGHFPAHREVMLSMLMHSSSKEV |
| | | FQASANALSTLLEQNVNFRKILLSKGIHLNVLELMQKHIHSPEVAE |
| | | SGCKMLNHLFEGSNTSLDIMAAVVPKILTVMKRHETSLPVQLEALR |
| | | AILHFIVPGMPEESREDTEFHHKLNMVKKQCFKNDIHKLVLAALNR |
| | | FIGNPGIQKCGLKVISSIVHFPDALEMLSLEGAMDSVLHTLQMYPD |
| | | DQEIQCLGLSLIGYLITKKNVFIGTGHLLAKILVSSLYRFKDVAEI |
| | | QTKGFQTILAILKLSASFSKLLVHHSFDLVIFHQMSSNIMEQKDQQ |
| | | FLNLCCKCFAKVAMDDYLKNVMLERACDQNNSIMVECLLLLGADAN |
| | | QAKEGSSLICQVCEKESSPKLVELLLNSGSREQDVRKALTISIGKG |
| | | DSQIISLLLRRLALDVANNSICLGGFCIGKVEPSWLGPLFPDKTSN |
| | | LRKQTNIASTLARMVIRYQMKSAVEEGTASGSDGNFSEDVLSKFDE |
| | | WTFIPDSSMDSVFAQSDDLDSEGSEGSFLVKKKSNSISVGEFYRDA |
| | | VLQRCSPNLQRHSNSLGPIFDHEDLLKRKRKILSSDDSLRSSKLQS |
| | | HMRHSDSISSLASEREYITSLDLSANELRDIDALSQKCCISVHLEH |
| | | LEKLELHQNALTSFPQQLCETLKSLTHLDLHSNKFTSFPSYLLKMS |
| | | CIANLDVSRNDIGPSVVLDPTVKCPTLKQFNLSYNQLSFVPENLTD |
| | | VVEKLEQLILEGNKISGICSPLRLKELKILNLSKNHISSLSENFLE |
| | | ACPKVESFSARMNFLAAMPFLPPSMTILKLSQNKFSCIPEAILNLP |
| | | HLRSLDMSSNDIQYLPGPAHWKSLNLRELLFSHNQISILDLSEKAY |
| | | LWSRVEKLHLSHNKLKEEQNEVTDNG |

Specifically, the LRRK2 polypeptide mutation G2019S has been suggested to play an important role in Parkinson's Disease in some ethnicities. The mutation can be autosomal dominant and the lifetime penetrance for the mutation has been estimated at about 31.8%. The SNP responsible for this missense mutation is annotated as rs34637584 in the human genome, and is a G to A substitution at the genomic level (6055G>A). This LRRK2 mutation can be referred to either as G2019S or 6055G>A and is found at or near chr12:40734202. The G2019S mutation has been shown to increase LRRK2 kinase activity, and is found in the within the activation domain or protein kinase-like domain of the protein. In some cases, a target amino acid residue to be corrected utilizing compositions provided herein can be residue 2019 of the LRRK2 polypeptide of SEQ ID NO: 15. Therefore, an engineered polynucleotide disclosed herein can target a region of a target RNA that comprises a sequence encoding the nucleotide codon that encodes the amino acid residue 2019 of the LRRK2 polypeptide of SEQ ID NO: 15. Additional exemplary amino acid residue mutations that can be reverted utilizing compositions and methods provided herein are shown in Table 3. Therefore, an engineered polynucleotide disclosed herein can target a region of a target RNA that comprises a sequence encoding nucleotide codon that encodes an amino acid residue mutation as shown in Table 3. In some embodiments, the engineered polynucleotide disclosed herein facilitates editing of a nucleotide of a codon that encodes an amino acid residue mutation, such as an amino acid residue mutation shown in Table 3. In some embodiments, the editing of a nucleotide of a codon that encodes an amino acid residue mutation results in a corrected amino acid residue upon translation of the edited codon.

TABLE 3
Exemplary protein mutations in LRRK2 isoform 1
and corresponding exons that can be targeted

| Exon number of LRRK2 | Protein Mutation of LRRK2 Isoform 1 |
|---|---|
| 1 | E10K |
| 2 | A30P; S52F |
| 3 | E46K; A53T |
| 4 | L119P |
| 6 | A211V; C228S |
| 9 | E334K; N363S; V366M |
| 11 | A419V |
| 13 | R506Q |
| 14 | K544E; N551K |
| 18 | A716V; M712V; I723V |
| 19 | P755L; R793M; I810V |
| 20 | K871E |
| 21 | Q923H; Q930R |
| 24 | R1067Q; S1096C; Q1111H |
| 25 | I1122V; A1151T; L1165P |
| 26 | I1192V |
| 27 | H1216R; S1228T |
| 28 | P1262A |
| 29 | R1325Q; I1371V |
| 30 | R1398H; T1410M; D1420N |
| 31 | R1441G; R1441C; R1441H; A1442P; P1446L; |
| | V1450I; K1468E; R1483Q |
| 32 | R1514Q; P1542S |
| 34 | V1613A; R1628P; M1646T; S1647T |
| 35 | Y1699C |
| 36 | R1728H; R1728L |
| 37 | L1795F |
| 38 | M1869V; M1869T; L1870F; E1874X |
| 40 | R1941H |
| 41 | Y2006H; I2012T; G2019S; I2020T; T2031S |
| 42 | N2081D |
| 44 | T2141M; R2143H; Y2189C |
| 48 | T2356I; G2385R; V2390M |
| 49 | E2395K; M2397T |
| 50 | L2466H |
| 51 | Q2490NfsX3 |

### Alpha-Synuclein (SNCA)

Alpha-synuclein is a major causative gene for familial Parkinson's Disease. Its aliases include NACP, PARK1, PARK4, PD1, synuclein alpha, or SNCA. The Alpha-synuclein gene is made up of 5 exons and encodes a 140 amino-acid protein with a predicted molecular mass of ˜14.5 kDa. The encoded product is an intrinsically disordered protein with unknown functions. Usually, Alpha-synuclein is a monomer. Under certain stress conditions or other unknown causes, α-synuclein self-aggregates into oligomers. Alpha-synuclein is highly expressed in the brain but is also found in the adrenal glands, appendix, bone marrow, colon, duodenum, endometrium, esophagus, fat, gall bladder, heart kidney, liver, lung, lymph node, ovary, placenta, prostate, skin, thyroid, bladder, skeletal muscle, and pancreas. In the brain, Alpha-synuclein is localized at the pre-synaptic terminal of the neuron and interacts with other proteins and phospholipids. The domain structure of Alpha-synuclein comprises an N-terminal A2 lipid-binding alpha-helix domain, a Non-amyloid β component (NAC) domain, and a C-terminal acidic domain. The lipid-binding domain consists of five KXKEGV imperfect repeats. The NAC domain consists of a GAV motif with a VGGAVVTGV consensus sequence and three GXXX sub-motifs—where X is any of Gly, Ala, Val, Ile, Leu, Phe, Tyr, Trp, Thr, Ser, or Met. The C-terminal acidic domain contains a copper-binding motif with a DPDNEA consensus sequence. Molecularly, Alpha-synuclein is suggested to play a role in neuronal transmission and DNA repair.

Pathological aggregates of α-synuclein are a defining characteristic of a group of diseases including Parkinson's Disease, Parkinson's Disease with Dementia (PDD), Dementia with Lewy Bodies (DLB), Multiple System Atrophy (MSA), and Pure Autonomic Failure (PAF). Five missense mutations—A30P, E46K, H50Q, G51D, A53E, and A53T—are causative of familial Parkinson's disease. These mutations are located within the N-terminal two alpha-helical regions. Other missense mutations, such as A18T, A29S, and A53V, have also been shown to be associated with Parkinson's Disease. Moreover, genome-wide association studies have identified many polymorphisms in Alpha-synuclein as risk factors for Parkinson's Disease. Copy-number variation, such as duplication, is also frequently found in many patients and a somatic mutation in some cases of Parkinson's Disease.

Alpha-synuclein can form “prion-like” aggregates and spread through connected neuronal networks. LRRK2 G2019S mutation has been shown to promote Alpha-synuclein aggregation in both mouse and human models. Alpha-synuclein aggregation is also reduced in the neurons with LRRK2 knocked out in vitro. The strong genetic interaction between Alpha-synuclein and LRRK2 and their important roles in Parkinson's Disease suggest that they are effective candidate targets for combinatorial therapy.

In some cases, a region of Alpha-synuclein can be targeted utilizing compositions provided herein. In some cases, a region of the Alpha-synuclein mRNA can be targeted by an engineered polynucleotide disclosed herein. In some cases, a region of the exon or intron of the Alpha-synuclein mRNA can be targeted by an engineered polynucleotide disclosed herein. In some embodiments, a region of the non-coding sequence of the Alpha-synuclein mRNA, such as the 5′UTR and 3′UTR, can be targeted by an engineered polynucleotide disclosed herein. In other cases, a region of the coding sequence of the Alpha-synuclein mRNA can be targeted by an engineered polynucleotide disclosed herein. In some cases, a polynucleotide comprises a targeting sequence that can hybridize to at least a portion of a sequence of Table 4. In some cases, a polynucleotide comprises a targeting sequence that can hybridize to at least a portion of a sequence that comprises at least about 80%, 85%, 90%, 95%, 97%, or 99% sequence identity to a sequence of Table 4. In some embodiments, a polynucleotide comprises a targeting sequence that can hybridize to at least a portion of a sequence that comprises at least about 80%, 85%, 90%, 95%, 97%, or 99% sequence identity to a sequence of Table 4. In other cases, a region of the coding sequence of the SNCA mRNA can be targeted by an engineered polynucleotide as described herein. In some cases, a region targeted by an engineered polynucleotide described herein comprises a region from a target RNA, wherein the target RNA comprises at least 80%, 85%, 90%, 95%, 97%, or 99% sequence identity to a sequence of Table 4. In some cases, a region targeted by an engineered polynucleotide described herein comprises a region from a target RNA, wherein the target RNA comprises at 100% sequence identity to a sequence of Table 4. Suitable regions include but are not limited to a N-terminal A2 lipid-binding alpha-helix domain, a Non-amyloid β component (NAC) domain, amino acid phosphorylation/glycosylation sites, or a C-terminal acidic domain. In some embodiments, a region of a target RNA is any region that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides in length, from any one of SEQ ID NO: 5 to SEQ ID NO: 14. In some embodiments, an engineered polynucleotide as described herein has at least 70%, 75%, 80%, 85%, 90%, 95%, 98%, 99%, or 100% complementarity to a region as described herein from a sequence of Table 4.

In some cases, a region of Alpha-synuclein can be targeted utilizing compositions provided herein. In some cases, a region of the Alpha-synuclein mRNA can be targeted with the engineered polynucleotides disclosed herein for knockdown. In some cases, a region of the exon or intron of the Alpha-synuclein mRNA can be targeted by an engineered polynucleotide disclosed herein. In some embodiments, a region of the non-coding sequence of the Alpha-synuclein mRNA, such as the 5′UTR and 3′UTR, can be targeted by an engineered polynucleotide disclosed herein. In other cases, a region of the coding sequence of the Alpha-synuclein mRNA can be targeted by an engineered polynucleotide disclosed herein. In some cases, a polynucleotide comprises a targeting sequence that can hybridize to at least a portion or region of a sequence of Table 4. In some cases, a polynucleotide comprises a targeting sequence that can hybridize to at least a portion or region of a sequence that comprises at least about 80%, 85%, 90%, 95%, 97%, or 99% sequence identity to a sequence of Table 4. In other cases, a region of the coding sequence of the SNCA mRNA can be targeted by an engineered polynucleotide as described herein. In some cases, a region targeted by an engineered polynucleotide described herein comprises a region from a target RNA, wherein the target RNA comprises at least 80%, 85%, 90%, 95%, 97%, or 99% sequence identity to a sequence of Table 4. In some cases, a region targeted by an engineered polynucleotide described herein comprises a region from a target RNA, wherein the target RNA comprises at 100% sequence identity to a sequence of Table 4. Suitable regions include but are not limited to a N-terminal A2 lipid-binding alpha-helix domain, a Non-amyloid β component (NAC) domain, or a C-terminal acidic domain. In some embodiments, a portion or a region of a target RNA is any portion or any region that is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 nucleotides in length, from any one of SEQ ID NO: 5 to SEQ ID NO: 14. In some embodiments, an engineered polynucleotide as described herein has at least 70%, 75%, 80%, 85%, 90%, 95%, 98%, 99%, or 100% complementarity to a region as described herein from a sequence of Table 4.

In some aspects, an alpha-synuclein mRNA sequence is targeted by an engineered polynucleotide as disclosed herein. Exemplary complete mRNA sequences are shown in Table 4. In some cases, any one of the 3,177 nucleotides of the sequence may be targeted utilizing the compositions and method provided herein. In some cases, a target nucleotide of the alpha-synuclein mRNA may be located among nucleotides 1-100, 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, 1901-2000, 2001-2100, 2101-2200, 2201-2300, 2301-2400, 2401-2500, 2501-2600, 2601-2700, 2701-2800, 2801-2900, 2901-3000, 3001-3100, and/or 3101-3177.

TABLE 4
Human Alpha-synuclein mRNA Isoform Sequences. Sequences derived from
NCBI SNCA sequence corresponding to gene ID 6622; Assembly GRCh38.p13
(GCF_000001405.39); NC_000004.12 (89724099 . . .89838324, complement).

| SEQ | | |
|---|---|---|
| ID NO: | Isoform | mRNA Sequence |
| 25 | Variant | GGCGACGACCAGAAGGGGCCCAAGAGAGGGGGCGAGCGACCGA |
| | 1 | GCGCCGCGACGCGGAAGTGAGGTGCGTGCGGGCTGCAGCGCAGA |
| | | CCCCGGCCCGGCCCCTCCGAGAGCGTCCTGGGCGCTCCCTCACGC |
| | | CTTGCCTTCAAGCCTTCTGCCTTTCCACCCTCGTGAGCGGAGAACT |
| | | GGGAGTGGCCATTCGACGACAGTGTGGTGTAAAGGAATTCATTAG |
| | | CCATGGATGTATTCATGAAAGGACTTTCAAAGGCCAAGGAGGGA |
| | | GTTGTGGCTGCTGCTGAGAAAACCAAACAGGGTGTGGCAGAAGC |
| | | AGCAGGAAAGACAAAAGAGGGTGTTCTCTATGTAGGCTCCAAAA |
| | | CCAAGGAGGGAGTGGTGCATGGTGTGGCAACAGTGGCTGAGAAG |
| | | ACCAAAGAGCAAGTGACAAATGTTGGAGGAGCAGTGGTGACGGG |
| | | TGTGACAGCAGTAGCCCAGAAGACAGTGGAGGGAGCAGGGAGCA |
| | | TTGCAGCAGCCACTGGCTTTGTCAAAAAGGACCAGTTGGGCAAGA |
| | | ATGAAGAAGGAGCCCCACAGGAAGGAATTCTGGAAGATATGCCT |
| | | GTGGATCCTGACAATGAGGCTTATGAAATGCCTTCTGAGGAAGGG |
| | | TATCAAGACTACGAACCTGAAGCCTAAGAAATATCTTTGCTCCCA |
| | | GTTTCTTGAGATCTGCTGACAGATGTTCCATCCTGTACAAGTGCTC |
| | | AGTTCCAATGTGCCCAGTCATGACATTTCTCAAAGTTTTTACAGTG |
| | | TATCTCGAAGTCTTCCATCAGCAGTGATTGAAGTATCTGTACCTGC |
| | | CCCCACTCAGCATTTCGGTGCTTCCCTTTCACTGAAGTGAATACAT |
| | | GGTAGCAGGGTCTTTGTGTGCTGTGGATTTTGTGGCTTCAATCTAC |
| | | GATGTTAAAACAAATTAAAAACACCTAAGTGACTACCACTTATTT |
| | | CTAAATCCTCACTATTTTTTTGTTGCTGTTGTTCAGAAGTTGTTAG |
| | | TGATTTGCTATCATATATTATAAGATTTTTAGGTGTCTTTTAATGA |
| | | TACTGTCTAAGAATAATGACGTATTGTGAAATTTGTTAATATATAT |
| | | AATACTTAAAAATATGTGAGCATGAAACTATGCACCTATAAATAC |
| | | TAAATATGAAATTTTACCATTTTGCGATGTGTTTTATTCACTTGTG |
| | | TTTGTATATAAATGGTGAGAATTAAAATAAAACGTTATCTCATTG |
| | | CAAAAATATTTTATTTTTATCCCATCTCACTTTAATAATAAAAATC |
| | | ATGCTTATAAGCAACATGAATTAAGAACTGACACAAAGGACAAA |
| | | AATATAAAGTTATTAATAGCCATTTGAAGAAGGAGGAATTTTAGA |
| | | AGAGGTAGAGAAAATGGAACATTAACCCTACACTCGGAATTCCCT |
| | | GAAGCAACACTGCCAGAAGTGTGTTTTGGTATGCACTGGTTCCTT |
| | | AAGTGGCTGTGATTAATTATTGAAAGTGGGGTGTTGAAGACCCCA |
| | | ACTACTATTGTAGAGTGGTCTATTTCTCCCTTCAATCCTGTCAATG |
| | | TTTGCTTTACGTATTTTGGGGAACTGTTGTTTGATGTGTATGTGTT |
| | | TATAATTGTTATACATTTTTAATTGAGCCTTTTATTAACATATATT |
| | | GTTATTTTTGTCTCGAAATAATTTTTTAGTTAAAATCTATTTTGTCT |
| | | GATATTGGTGTGAATGCTGTACCTTTCTGACAATAAATAATATTC |
| | | GACCATGAATAAAAAAAAAAAAAAAGTGGGTTCCCGGGAACTAA |
| | | GCAGTGTAGAAGATGATTTTGACTACACCCTCCTTAGAGAGCCAT |
| | | AAGACACATTAGCACATATTAGCACATTCAAGGCTCTGAGAGAAT |
| | | GTGGTTAACTTTGTTTAACTCAGCATTCCTCACTTTTTTTTTTTAAT |
| | | CATCAGAAATTCTCTCTCTCTCTCTCTCTTTTTCTCTCGCTCTCTTT |
| | | TTTTTTTTTTTTTTACAGGAAATGCCTTTAAACATCGTTGGAACTA |
| | | CCAGAGTCACCTTAAAGGAGATCAATTCTCTAGACTGATAAAAAT |
| | | TTCATGGCCTCCTTTAAATGTTGCCAAATATATGAATTCTAGGATT |
| | | TTTCCTTAGGAAAGGTTTTTCTCTTTCAGGGAAGATCTATTAACTC |
| | | CCCATGGGTGCTGAAAATAAACTTGATGGTGAAAAACTCTGTATA |
| | | AATTAATTTAAAAATTATTTGGTTTCTCTTTTTAATTATTCTGGGG |
| | | CATAGTCATTTCTAAAAGTCACTAGTAGAAAGTATAATTTCAAGA |
| | | CAGAATATTCTAGACATGCTAGCAGTTTATATGTATTCATGAGTA |
| | | ATGTGATATATATTGGGCGCTGGTGAGGAAGGAAGGAGGAATGA |
| | | GTGACTATAAGGATGGTTACCATAGAAACTTCCTTTTTTACCTAAT |
| | | TGAAGAGAGACTACTACAGAGTGCTAAGCTGCATGTGTCATCTTA |
| | | CACTAGAGAGAAATGGTAAGTTTCTTGTTTTATTTAAGTTATGTTT |
| | | AAGCAAGGAAAGGATTTGTTATTGAACAGTATATTTCAGGAAGGT |
| | | TAGAAAGTGGCGGTTAGGATATATTTTAAATCTACCTAAAGCAGC |
| | | ATATTTTAAAAATTTAAAAGTATTGGTATTAAATTAAGAAATAGA |
| | | GGACAGAACTAGACTGATAGCAGTGACCTAGAACAATTTGAGAT |
| | | TAGGAAAGTTGTGACCATGAATTTAAGGATTTATGTGGATACAAA |
| | | TTCTCCTTTAAAGTGTTTCTTCCCTTAATATTTATCTGACGGTAATT |
| | | TTTGAGCAGTGAATTACTTTATATATCTTAATAGTTTATTTGGGAC |
| | | CAAACACTTAAACAAAAAGTTCTTTAAGTCATATAAGCCTTTTCA |
| | | GGAAGCTTGTCTCATATTCACTCCCGAGACATTCACCTGCCAAGT |
| | | GGCCTGAGGATCAATCCAGTCCTAGGTTTATTTTGCAGACTTACA |
| | | TTCTCCCAAGTTATTCAGCCTCATATGACTCCACGGTCGGCTTTAC |
| | | CAAAACAGTTCAGAGTGCACTTTGGCACACAATTGGGAACAGAA |
| | | CAATCTAATGTGTGGTTTGGTATTCCAAGTGGGGTCTTTTTCAGAA |
| | | TCTCTGCACTAGTGTGAGATGCAAACATGTTTCCTCATCTTTCTGG |
| | | CTTATCCAGTATGTAGCTATTTGTGACATAATAAATATATACATAT |
| | | ATGAAAATA |
| 26 | Variant | GGCGACGACCAGAAGGGGCCCAAGAGAGGGGGCGAGCGACCGA |
| | 2 | GCGCCGCGACGCGGAAGTGAGGTGCGTGCGGGCTGCAGCGCAGA |
| | | CCCCGGCCCGGCCCCTCCGAGAGCGTCCTGGGCGCTCCCTCACGC |
| | | CTTGCCTTCAAGCCTTCTGCCTTTCCACCCTCGTGAGCGGAGAACT |
| | | GGGAGTGGCCATTCGACGACAGGTTAGCGGGTTTGCCTCCCACTC |
| | | CCCCAGCCTCGCGTCGCCGGCTCACAGCGGCCTCCTCTGGGGACA |
| | | GTCCCCCCCGGGTGCCGCCTCCGCCCTTCCTGTGCGCTCCTTTTCC |
| | | TTCTTCTTTCCTATTAAATATTATTTGGGAATTGTTTAAATTTTTTT |
| | | TTTAAAAAAAGAGAGAGGCGGGGAGGAGTCGGAGTTGTGGAGAA |
| | | GCAGAGGGACTCAGTGTGGTGTAAAGGAATTCATTAGCCATGGAT |
| | | GTATTCATGAAAGGACTTTCAAAGGCCAAGGAGGGAGTTGTGGCT |
| | | GCTGCTGAGAAAACCAAACAGGGTGTGGCAGAAGCAGCAGGAAA |
| | | GACAAAAGAGGGTGTTCTCTATGTAGGCTCCAAAACCAAGGAGG |
| | | GAGTGGTGCATGGTGTGGCAACAGTGGCTGAGAAGACCAAAGAG |
| | | CAAGTGACAAATGTTGGAGGAGCAGTGGTGACGGGTGTGACAGC |
| | | AGTAGCCCAGAAGACAGTGGAGGGAGCAGGGAGCATTGCAGCAG |
| | | CCACTGGCTTTGTCAAAAAGGACCAGTTGGGCAAGAATGAAGAA |
| | | GGAGCCCCACAGGAAGGAATTCTGGAAGATATGCCTGTGGATCCT |
| | | GACAATGAGGCTTATGAAATGCCTTCTGAGGAAGGGTATCAAGA |
| | | CTACGAACCTGAAGCCTAAGAAATATCTTTGCTCCCAGTTTCTTG |
| | | AGATCTGCTGACAGATGTTCCATCCTGTACAAGTGCTCAGTTCCA |
| | | ATGTGCCCAGTCATGACATTTCTCAAAGTTTTTACAGTGTATCTCG |
| | | AAGTCTTCCATCAGCAGTGATTGAAGTATCTGTACCTGCCCCCAC |
| | | TCAGCATTTCGGTGCTTCCCTTTCACTGAAGTGAATACATGGTAGC |
| | | AGGGTCTTTGTGTGCTGTGGATTTTGTGGCTTCAATCTACGATGTT |
| | | AAAACAAATTAAAAACACCTAAGTGACTACCACTTATTTCTAAAT |
| | | CCTCACTATTTTTTTGTTGCTGTTGTTCAGAAGTTGTTAGTGATTTG |
| | | CTATCATATATTATAAGATTTTTAGGTGTCTTTTAATGATACTGTC |
| | | TAAGAATAATGACGTATTGTGAAATTTGTTAATATATATAATACT |
| | | TAAAAATATGTGAGCATGAAACTATGCACCTATAAATACTAAATA |
| | | TGAAATTTTACCATTTTGCGATGTGTTTTATTCACTTGTGTTTGTAT |
| | | ATAAATGGTGAGAATTAAAATAAAACGTTATCTCATTGCAAAAAT |
| | | ATTTTATTTTTATCCCATCTCACTTTAATAATAAAAATCATGCTTA |
| | | TAAGCAACATGAATTAAGAACTGACACAAAGGACAAAAATATAA |
| | | AGTTATTAATAGCCATTTGAAGAAGGAGGAATTTTAGAAGAGGTA |
| | | GAGAAAATGGAACATTAACCCTACACTCGGAATTCCCTGAAGCA |
| | | ACACTGCCAGAAGTGTGTTTTGGTATGCACTGGTTCCTTAAGTGG |
| | | CTGTGATTAATTATTGAAAGTGGGGTGTTGAAGACCCCAACTACT |
| | | ATTGTAGAGTGGTCTATTTCTCCCTTCAATCCTGTCAATGTTTGCT |
| | | TTACGTATTTTGGGGAACTGTTGTTTGATGTGTATGTGTTTATAAT |
| | | TGTTATACATTTTTAATTGAGCCTTTTATTAACATATATTGTTATTT |
| | | TTGTCTCGAAATAATTTTTTAGTTAAAATCTATTTTGTCTGATATT |
| | | GGTGTGAATGCTGTACCTTTCTGACAATAAATAATATTCGACCAT |
| | | GAATAAAAAAAAAAAAAAAGTGGGTTCCCGGGAACTAAGCAGTG |
| | | TAGAAGATGATTTTGACTACACCCTCCTTAGAGAGCCATAAGACA |
| | | CATTAGCACATATTAGCACATTCAAGGCTCTGAGAGAATGTGGTT |
| | | AACTTTGTTTAACTCAGCATTCCTCACTTTTTTTTTTTAATCATCAG |
| | | AAATTCTCTCTCTCTCTCTCTCTTTTTCTCTCGCTCTCTTTTTTTTTT |
| | | TTTTTTTACAGGAAATGCCTTTAAACATCGTTGGAACTACCAGAG |
| | | TCACCTTAAAGGAGATCAATTCTCTAGACTGATAAAAATTTCATG |
| | | GCCTCCTTTAAATGTTGCCAAATATATGAATTCTAGGATTTTTCCT |
| | | TAGGAAAGGTTTTTCTCTTTCAGGGAAGATCTATTAACTCCCCATG |
| | | GGTGCTGAAAATAAACTTGATGGTGAAAAACTCTGTATAAATTAA |
| | | TTTAAAAATTATTTGGTTTCTCTTTTTAATTATTCTGGGGCATAGT |
| | | CATTTCTAAAAGTCACTAGTAGAAAGTATAATTTCAAGACAGAAT |
| | | ATTCTAGACATGCTAGCAGTTTATATGTATTCATGAGTAATGTGAT |
| | | ATATATTGGGCGCTGGTGAGGAAGGAAGGAGGAATGAGTGACTA |
| | | TAAGGATGGTTACCATAGAAACTTCCTTTTTTACCTAATTGAAGA |
| | | GAGACTACTACAGAGTGCTAAGCTGCATGTGTCATCTTACACTAG |
| | | AGAGAAATGGTAAGTTTCTTGTTTTATTTAAGTTATGTTTAAGCAA |
| | | GGAAAGGATTTGTTATTGAACAGTATATTTCAGGAAGGTTAGAAA |
| | | GTGGCGGTTAGGATATATTTTAAATCTACCTAAAGCAGCATATTT |
| | | TAAAAATTTAAAAGTATTGGTATTAAATTAAGAAATAGAGGACA |
| | | GAACTAGACTGATAGCAGTGACCTAGAACAATTTGAGATTAGGA |
| | | AAGTTGTGACCATGAATTTAAGGATTTATGTGGATACAAATTCTC |
| | | CTTTAAAGTGTTTCTTCCCTTAATATTTATCTGACGGTAATTTTTG |
| | | AGCAGTGAATTACTTTATATATCTTAATAGTTTATTTGGGACCAAA |
| | | CACTTAAACAAAAAGTTCTTTAAGTCATATAAGCCTTTTCAGGAA |
| | | GCTTGTCTCATATTCACTCCCGAGACATTCACCTGCCAAGTGGCCT |
| | | GAGGATCAATCCAGTCCTAGGTTTATTTTGCAGACTTACATTCTCC |
| | | CAAGTTATTCAGCCTCATATGACTCCACGGTCGGCTTTACCAAAA |
| | | CAGTTCAGAGTGCACTTTGGCACACAATTGGGAACAGAACAATCT |
| | | AATGTGTGGTTTGGTATTCCAAGTGGGGTCTTTTTCAGAATCTCTG |
| | | CACTAGTGTGAGATGCAAACATGTTTCCTCATCTTTCTGGCTTATC |
| | | CAGTATGTAGCTATTTGTGACATAATAAATATATACATATATGAA |
| | | AATA |
| 27 | Variant | GCTTCTCCATTCTGGTGTGATCCAGGAACAGCTGTCTTCCAGCTCT |
| | 3 | GAAAGAGTGTGGTGTAAAGGAATTCATTAGCCATGGATGTATTCA |
| | | TGAAAGGACTTTCAAAGGCCAAGGAGGGAGTTGTGGCTGCTGCT |
| | | GAGAAAACCAAACAGGGTGTGGCAGAAGCAGCAGGAAAGACAA |
| | | AAGAGGGTGTTCTCTATGTAGGCTCCAAAACCAAGGAGGGAGTG |
| | | GTGCATGGTGTGGCAACAGTGGCTGAGAAGACCAAAGAGCAAGT |
| | | GACAAATGTTGGAGGAGCAGTGGTGACGGGTGTGACAGCAGTAG |
| | | CCCAGAAGACAGTGGAGGGAGCAGGGAGCATTGCAGCAGCCACT |
| | | GGCTTTGTCAAAAAGGACCAGTTGGGCAAGAATGAAGAAGGAGC |
| | | CCCACAGGAAGGAATTCTGGAAGATATGCCTGTGGATCCTGACAA |
| | | TGAGGCTTATGAAATGCCTTCTGAGGAAGGGTATCAAGACTACGA |
| | | ACCTGAAGCCTAAGAAATATCTTTGCTCCCAGTTTCTTGAGATCTG |
| | | CTGACAGATGTTCCATCCTGTACAAGTGCTCAGTTCCAATGTGCC |
| | | CAGTCATGACATTTCTCAAAGTTTTTACAGTGTATCTCGAAGTCTT |
| | | CCATCAGCAGTGATTGAAGTATCTGTACCTGCCCCCACTCAGCAT |
| | | TTCGGTGCTTCCCTTTCACTGAAGTGAATACATGGTAGCAGGGTC |
| | | TTTGTGTGCTGTGGATTTTGTGGCTTCAATCTACGATGTTAAAACA |
| | | AATTAAAAACACCTAAGTGACTACCACTTATTTCTAAATCCTCAC |
| | | TATTTTTTTGTTGCTGTTGTTCAGAAGTTGTTAGTGATTTGCTATCA |
| | | TATATTATAAGATTTTTAGGTGTCTTTTAATGATACTGTCTAAGAA |
| | | TAATGACGTATTGTGAAATTTGTTAATATATATAATACTTAAAAA |
| | | TATGTGAGCATGAAACTATGCACCTATAAATACTAAATATGAAAT |
| | | TTTACCATTTTGCGATGTGTTTTATTCACTTGTGTTTGTATATAAAT |
| | | GGTGAGAATTAAAATAAAACGTTATCTCATTGCAAAAATATTTTA |
| | | TTTTTATCCCATCTCACTTTAATAATAAAAATCATGCTTATAAGCA |
| | | ACATGAATTAAGAACTGACACAAAGGACAAAAATATAAAGTTAT |
| | | TAATAGCCATTTGAAGAAGGAGGAATTTTAGAAGAGGTAGAGAA |
| | | AATGGAACATTAACCCTACACTCGGAATTCCCTGAAGCAACACTG |
| | | CCAGAAGTGTGTTTTGGTATGCACTGGTTCCTTAAGTGGCTGTGAT |
| | | TAATTATTGAAAGTGGGGTGTTGAAGACCCCAACTACTATTGTAG |
| | | AGTGGTCTATTTCTCCCTTCAATCCTGTCAATGTTTGCTTTACGTA |
| | | TTTTGGGGAACTGTTGTTTGATGTGTATGTGTTTATAATTGTTATA |
| | | CATTTTTAATTGAGCCTTTTATTAACATATATTGTTATTTTTGTCTC |
| | | GAAATAATTTTTTAGTTAAAATCTATTTTGTCTGATATTGGTGTGA |
| | | ATGCTGTACCTTTCTGACAATAAATAATATTCGACCATGAATAAA |
| | | AAAAAAAAAAAAGTGGGTTCCCGGGAACTAAGCAGTGTAGAAGA |
| | | TGATTTTGACTACACCCTCCTTAGAGAGCCATAAGACACATTAGC |
| | | ACATATTAGCACATTCAAGGCTCTGAGAGAATGTGGTTAACTTTG |
| | | TTTAACTCAGCATTCCTCACTTTTTTTTTTTAATCATCAGAAATTCT |
| | | CTCTCTCTCTCTCTCTTTTTCTCTCGCTCTCTTTTTTTTTTTTTTTTTA |
| | | CAGGAAATGCCTTTAAACATCGTTGGAACTACCAGAGTCACCTTA |
| | | AAGGAGATCAATTCTCTAGACTGATAAAAATTTCATGGCCTCCTT |
| | | TAAATGTTGCCAAATATATGAATTCTAGGATTTTTCCTTAGGAAA |
| | | GGTTTTTCTCTTTCAGGGAAGATCTATTAACTCCCCATGGGTGCTG |
| | | AAAATAAACTTGATGGTGAAAAACTCTGTATAAATTAATTTAAAA |
| | | ATTATTTGGTTTCTCTTTTTAATTATTCTGGGGCATAGTCATTTCTA |
| | | AAAGTCACTAGTAGAAAGTATAATTTCAAGACAGAATATTCTAGA |
| | | CATGCTAGCAGTTTATATGTATTCATGAGTAATGTGATATATATTG |
| | | GGCGCTGGTGAGGAAGGAAGGAGGAATGAGTGACTATAAGGATG |
| | | GTTACCATAGAAACTTCCTTTTTTACCTAATTGAAGAGAGACTACT |
| | | ACAGAGTGCTAAGCTGCATGTGTCATCTTACACTAGAGAGAAATG |
| | | GTAAGTTTCTTGTTTTATTTAAGTTATGTTTAAGCAAGGAAAGGAT |
| | | TTGTTATTGAACAGTATATTTCAGGAAGGTTAGAAAGTGGCGGTT |
| | | AGGATATATTTTAAATCTACCTAAAGCAGCATATTTTAAAAATTT |
| | | AAAAGTATTGGTATTAAATTAAGAAATAGAGGACAGAACTAGAC |
| | | TGATAGCAGTGACCTAGAACAATTTGAGATTAGGAAAGTTGTGAC |
| | | CATGAATTTAAGGATTTATGTGGATACAAATTCTCCTTTAAAGTGT |
| | | TTCTTCCCTTAATATTTATCTGACGGTAATTTTTGAGCAGTGAATT |
| | | ACTTTATATATCTTAATAGTTTATTTGGGACCAAACACTTAAACAA |
| | | AAAGTTCTTTAAGTCATATAAGCCTTTTCAGGAAGCTTGTCTCATA |
| | | TTCACTCCCGAGACATTCACCTGCCAAGTGGCCTGAGGATCAATC |
| | | CAGTCCTAGGTTTATTTTGCAGACTTACATTCTCCCAAGTTATTCA |
| | | GCCTCATATGACTCCACGGTCGGCTTTACCAAAACAGTTCAGAGT |
| | | GCACTTTGGCACACAATTGGGAACAGAACAATCTAATGTGTGGTT |
| | | TGGTATTCCAAGTGGGGTCTTTTTCAGAATCTCTGCACTAGTGTGA |
| | | GATGCAAACATGTTTCCTCATCTTTCTGGCTTATCCAGTATGTAGC |
| | | TATTTGTGACATAATAAATATATACATATATGAAAATA |
| 28 | Variant | GGCGACGACCAGAAGGGGCCCAAGAGAGGGGGCGAGCGACCGA |
| | 4 | GCGCCGCGACGCGGAAGTGAGGTGCGTGCGGGCTGCAGCGCAGA |
| | | CCCCGGCCCGGCCCCTCCGAGAGCGTCCTGGGCGCTCCCTCACGC |
| | | CTTGCCTTCAAGCCTTCTGCCTTTCCACCCTCGTGAGCGGAGAACT |
| | | GGGAGTGGCCATTCGACGACAGGTTAGCGGGTTTGCCTCCCACTC |
| | | CCCCAGCCTCGCGTCGCCGGCTCACAGCGGCCTCCTCTGGGGACA |
| | | GTCCCCCCCGGGTGCCGCCTCCGCCCTTCCTGTGCGCTCCTTTTCC |
| | | TTCTTCTTTCCTATTAAATATTATTTGGGAATTGTTTAAATTTTTTT |
| | | TTTAAAAAAAGAGAGAGGCGGGGAGGAGTCGGAGTTGTGGAGAA |
| | | GCAGAGGGACTCAGTGTGGTGTAAAGGAATTCATTAGCCATGGAT |
| | | GTATTCATGAAAGGACTTTCAAAGGCCAAGGAGGGAGTTGTGGCT |
| | | GCTGCTGAGAAAACCAAACAGGGTGTGGCAGAAGCAGCAGGAAA |
| | | GACAAAAGAGGGTGTTCTCTATGTAGGCTCCAAAACCAAGGAGG |
| | | GAGTGGTGCATGGTGTGGCAACAGTGGCTGAGAAGACCAAAGAG |
| | | CAAGTGACAAATGTTGGAGGAGCAGTGGTGACGGGTGTGACAGC |
| | | AGTAGCCCAGAAGACAGTGGAGGGAGCAGGGAGCATTGCAGCAG |
| | | CCACTGGCTTTGTCAAAAAGGACCAGTTGGGCAAGGAAGGGTAT |
| | | CAAGACTACGAACCTGAAGCCTAAGAAATATCTTTGCTCCCAGTT |
| | | TCTTGAGATCTGCTGACAGATGTTCCATCCTGTACAAGTGCTCAGT |
| | | TCCAATGTGCCCAGTCATGACATTTCTCAAAGTTTTTACAGTGTAT |
| | | CTCGAAGTCTTCCATCAGCAGTGATTGAAGTATCTGTACCTGCCC |
| | | CCACTCAGCATTTCGGTGCTTCCCTTTCACTGAAGTGAATACATGG |
| | | TAGCAGGGTCTTTGTGTGCTGTGGATTTTGTGGCTTCAATCTACGA |
| | | TGTTAAAACAAATTAAAAACACCTAAGTGACTACCACTTATTTCT |
| | | AAATCCTCACTATTTTTTTGTTGCTGTTGTTCAGAAGTTGTTAGTG |
| | | ATTTGCTATCATATATTATAAGATTTTTAGGTGTCTTTTAATGATA |
| | | CTGTCTAAGAATAATGACGTATTGTGAAATTTGTTAATATATATA |
| | | ATACTTAAAAATATGTGAGCATGAAACTATGCACCTATAAATACT |
| | | AAATATGAAATTTTACCATTTTGCGATGTGTTTTATTCACTTGTGT |
| | | TTGTATATAAATGGTGAGAATTAAAATAAAACGTTATCTCATTGC |
| | | AAAAATATTTTATTTTTATCCCATCTCACTTTAATAATAAAAATCA |
| | | TGCTTATAAGCAACATGAATTAAGAACTGACACAAAGGACAAAA |
| | | ATATAAAGTTATTAATAGCCATTTGAAGAAGGAGGAATTTTAGAA |
| | | GAGGTAGAGAAAATGGAACATTAACCCTACACTCGGAATTCCCTG |
| | | AAGCAACACTGCCAGAAGTGTGTTTTGGTATGCACTGGTTCCTTA |
| | | AGTGGCTGTGATTAATTATTGAAAGTGGGGTGTTGAAGACCCCAA |
| | | CTACTATTGTAGAGTGGTCTATTTCTCCCTTCAATCCTGTCAATGT |
| | | TTGCTTTACGTATTTTGGGGAACTGTTGTTTGATGTGTATGTGTTT |
| | | ATAATTGTTATACATTTTTAATTGAGCCTTTTATTAACATATATTG |
| | | TTATTTTTGTCTCGAAATAATTTTTTAGTTAAAATCTATTTTGTCTG |
| | | ATATTGGTGTGAATGCTGTACCTTTCTGACAATAAATAATATTCG |
| | | ACCATGAATAAAAAAAAAAAAAAAGTGGGTTCCCGGGAACTAAG |
| | | CAGTGTAGAAGATGATTTTGACTACACCCTCCTTAGAGAGCCATA |
| | | AGACACATTAGCACATATTAGCACATTCAAGGCTCTGAGAGAATG |
| | | TGGTTAACTTTGTTTAACTCAGCATTCCTCACTTTTTTTTTTTAATC |
| | | ATCAGAAATTCTCTCTCTCTCTCTCTCTTTTTCTCTCGCTCTCTTTT |
| | | TTTTTTTTTTTTTACAGGAAATGCCTTTAAACATCGTTGGAACTAC |
| | | CAGAGTCACCTTAAAGGAGATCAATTCTCTAGACTGATAAAAATT |
| | | TCATGGCCTCCTTTAAATGTTGCCAAATATATGAATTCTAGGATTT |
| | | TTCCTTAGGAAAGGTTTTTCTCTTTCAGGGAAGATCTATTAACTCC |
| | | CCATGGGTGCTGAAAATAAACTTGATGGTGAAAAACTCTGTATAA |
| | | ATTAATTTAAAAATTATTTGGTTTCTCTTTTTAATTATTCTGGGGC |
| | | ATAGTCATTTCTAAAAGTCACTAGTAGAAAGTATAATTTCAAGAC |
| | | AGAATATTCTAGACATGCTAGCAGTTTATATGTATTCATGAGTAA |
| | | TGTGATATATATTGGGCGCTGGTGAGGAAGGAAGGAGGAATGAG |
| | | TGACTATAAGGATGGTTACCATAGAAACTTCCTTTTTTACCTAATT |
| | | GAAGAGAGACTACTACAGAGTGCTAAGCTGCATGTGTCATCTTAC |
| | | ACTAGAGAGAAATGGTAAGTTTCTTGTTTTATTTAAGTTATGTTTA |
| | | AGCAAGGAAAGGATTTGTTATTGAACAGTATATTTCAGGAAGGTT |
| | | AGAAAGTGGCGGTTAGGATATATTTTAAATCTACCTAAAGCAGCA |
| | | TATTTTAAAAATTTAAAAGTATTGGTATTAAATTAAGAAATAGAG |
| | | GACAGAACTAGACTGATAGCAGTGACCTAGAACAATTTGAGATT |
| | | AGGAAAGTTGTGACCATGAATTTAAGGATTTATGTGGATACAAAT |
| | | TCTCCTTTAAAGTGTTTCTTCCCTTAATATTTATCTGACGGTAATTT |
| | | TTGAGCAGTGAATTACTTTATATATCTTAATAGTTTATTTGGGACC |
| | | AAACACTTAAACAAAAAGTTCTTTAAGTCATATAAGCCTTTTCAG |
| | | GAAGCTTGTCTCATATTCACTCCCGAGACATTCACCTGCCAAGTG |
| | | GCCTGAGGATCAATCCAGTCCTAGGTTTATTTTGCAGACTTACATT |
| | | CTCCCAAGTTATTCAGCCTCATATGACTCCACGGTCGGCTTTACCA |
| | | AAACAGTTCAGAGTGCACTTTGGCACACAATTGGGAACAGAACA |
| | | ATCTAATGTGTGGTTTGGTATTCCAAGTGGGGTCTTTTTCAGAATC |
| | | TCTGCACTAGTGTGAGATGCAAACATGTTTCCTCATCTTTCTGGCT |
| | | TATCCAGTATGTAGCTATTTGTGACATAATAAATATATACATATAT |
| | | GAAAATA |
| 29 | Variant | GCTTCTCCATTCTGGTGTGATCCAGGAACAGCTGTCTTCCAGCTCT |
| | 5 | GAAAGAGGGCTGAGAGATTAGGCTGCTTCTCCGGGATCCGCTTTT |
| | | CCCCGGGAAACGCGAGGATGCTCCATGGAGCTGTGGTGTAAAGG |
| | | AATTCATTAGCCATGGATGTATTCATGAAAGGACTTTCAAAGGCC |
| | | AAGGAGGGAGTTGTGGCTGCTGCTGAGAAAACCAAACAGGGTGT |
| | | GGCAGAAGCAGCAGGAAAGACAAAAGAGGGTGTTCTCTATGTAG |
| | | GCTCCAAAACCAAGGAGGGAGTGGTGCATGGTGTGGCAACAGTG |
| | | GCTGAGAAGACCAAAGAGCAAGTGACAAATGTTGGAGGAGCAGT |
| | | GGTGACGGGTGTGACAGCAGTAGCCCAGAAGACAGTGGAGGGAG |
| | | CAGGGAGCATTGCAGCAGCCACTGGCTTTGTCAAAAAGGACCAG |
| | | TTGGGCAAGAATGAAGAAGGAGCCCCACAGGAAGGAATTCTGGA |
| | | AGATATGCCTGTGGATCCTGACAATGAGGCTTATGAAATGCCTTC |
| | | TGAGGAAGGGTATCAAGACTACGAACCTGAAGCCTAAGAAATAT |
| | | CTTTGCTCCCAGTTTCTTGAGATCTGCTGACAGATGTTCCATCCTG |
| | | TACAAGTGCTCAGTTCCAATGTGCCCAGTCATGACATTTCTCAAA |
| | | GTTTTTACAGTGTATCTCGAAGTCTTCCATCAGCAGTGATTGAAGT |
| | | ATCTGTACCTGCCCCCACTCAGCATTTCGGTGCTTCCCTTTCACTG |
| | | AAGTGAATACATGGTAGCAGGGTCTTTGTGTGCTGTGGATTTTGT |
| | | GGCTTCAATCTACGATGTTAAAACAAATTAAAAACACCTAAGTGA |
| | | CTACCACTTATTTCTAAATCCTCACTATTTTTTTGTTGCTGTTGTTC |
| | | AGAAGTTGTTAGTGATTTGCTATCATATATTATAAGATTTTTAGGT |
| | | GTCTTTTAATGATACTGTCTAAGAATAATGACGTATTGTGAAATTT |
| | | GTTAATATATATAATACTTAAAAATATGTGAGCATGAAACTATGC |
| | | ACCTATAAATACTAAATATGAAATTTTACCATTTTGCGATGTGTTT |
| | | TATTCACTTGTGTTTGTATATAAATGGTGAGAATTAAAATAAAAC |
| | | GTTATCTCATTGCAAAAATATTTTATTTTTATCCCATCTCACTTTA |
| | | ATAATAAAAATCATGCTTATAAGCAACATGAATTAAGAACTGACA |
| | | CAAAGGACAAAAATATAAAGTTATTAATAGCCATTTGAAGAAGG |
| | | AGGAATTTTAGAAGAGGTAGAGAAAATGGAACATTAACCCTACA |
| | | CTCGGAATTCCCTGAAGCAACACTGCCAGAAGTGTGTTTTGGTAT |
| | | GCACTGGTTCCTTAAGTGGCTGTGATTAATTATTGAAAGTGGGGT |
| | | GTTGAAGACCCCAACTACTATTGTAGAGTGGTCTATTTCTCCCTTC |
| | | AATCCTGTCAATGTTTGCTTTACGTATTTTGGGGAACTGTTGTTTG |
| | | ATGTGTATGTGTTTATAATTGTTATACATTTTTAATTGAGCCTTTT |
| | | ATTAACATATATTGTTATTTTTGTCTCGAAATAATTTTTTAGTTAA |
| | | AATCTATTTTGTCTGATATTGGTGTGAATGCTGTACCTTTCTGACA |
| | | ATAAATAATATTCGACCATGAATAAAAAAAAAAAAAAAGTGGGT |
| | | TCCCGGGAACTAAGCAGTGTAGAAGATGATTTTGACTACACCCTC |
| | | CTTAGAGAGCCATAAGACACATTAGCACATATTAGCACATTCAAG |
| | | GCTCTGAGAGAATGTGGTTAACTTTGTTTAACTCAGCATTCCTCAC |
| | | TTTTTTTTTTTAATCATCAGAAATTCTCTCTCTCTCTCTCTCTTTTTC |
| | | TCTCGCTCTCTTTTTTTTTTTTTTTTTACAGGAAATGCCTTTAAACA |
| | | TCGTTGGAACTACCAGAGTCACCTTAAAGGAGATCAATTCTCTAG |
| | | ACTGATAAAAATTTCATGGCCTCCTTTAAATGTTGCCAAATATAT |
| | | GAATTCTAGGATTTTTCCTTAGGAAAGGTTTTTCTCTTTCAGGGAA |
| | | GATCTATTAACTCCCCATGGGTGCTGAAAATAAACTTGATGGTGA |
| | | AAAACTCTGTATAAATTAATTTAAAAATTATTTGGTTTCTCTTTTT |
| | | AATTATTCTGGGGCATAGTCATTTCTAAAAGTCACTAGTAGAAAG |
| | | TATAATTTCAAGACAGAATATTCTAGACATGCTAGCAGTTTATAT |
| | | GTATTCATGAGTAATGTGATATATATTGGGCGCTGGTGAGGAAGG |
| | | AAGGAGGAATGAGTGACTATAAGGATGGTTACCATAGAAACTTC |
| | | CTTTTTTACCTAATTGAAGAGAGACTACTACAGAGTGCTAAGCTG |
| | | CATGTGTCATCTTACACTAGAGAGAAATGGTAAGTTTCTTGTTTTA |
| | | TTTAAGTTATGTTTAAGCAAGGAAAGGATTTGTTATTGAACAGTA |
| | | TATTTCAGGAAGGTTAGAAAGTGGCGGTTAGGATATATTTTAAAT |
| | | CTACCTAAAGCAGCATATTTTAAAAATTTAAAAGTATTGGTATTA |
| | | AATTAAGAAATAGAGGACAGAACTAGACTGATAGCAGTGACCTA |
| | | GAACAATTTGAGATTAGGAAAGTTGTGACCATGAATTTAAGGATT |
| | | TATGTGGATACAAATTCTCCTTTAAAGTGTTTCTTCCCTTAATATT |
| | | TATCTGACGGTAATTTTTGAGCAGTGAATTACTTTATATATCTTAA |
| | | TAGTTTATTTGGGACCAAACACTTAAACAAAAAGTTCTTTAAGTC |
| | | ATATAAGCCTTTTCAGGAAGCTTGTCTCATATTCACTCCCGAGAC |
| | | ATTCACCTGCCAAGTGGCCTGAGGATCAATCCAGTCCTAGGTTTA |
| | | TTTTGCAGACTTACATTCTCCCAAGTTATTCAGCCTCATATGACTC |
| | | CACGGTCGGCTTTACCAAAACAGTTCAGAGTGCACTTTGGCACAC |
| | | AATTGGGAACAGAACAATCTAATGTGTGGTTTGGTATTCCAAGTG |
| | | GGGTCTTTTTCAGAATCTCTGCACTAGTGTGAGATGCAAACATGT |
| | | TTCCTCATCTTTCTGGCTTATCCAGTATGTAGCTATTTGTGACATA |
| | | ATAAATATATACATATATGAAAATA |
| 30 | Variant | GGCGACGACCAGAAGGGGCCCAAGAGAGGGGGCGAGCGACCGA |
| | 6 | GCGCCGCGACGCGGAAGTGAGTGTGGTGTAAAGGAATTCATTAG |
| | | CCATGGATGTATTCATGAAAGGACTTTCAAAGGCCAAGGAGGGA |
| | | GTTGTGGCTGCTGCTGAGAAAACCAAACAGGGTGTGGCAGAAGC |
| | | AGCAGGAAAGACAAAAGAGGGTGTTCTCTATGTAGGCTCCAAAA |
| | | CCAAGGAGGGAGTGGTGCATGGTGTGGCAACAGTGGCTGAGAAG |
| | | ACCAAAGAGCAAGTGACAAATGTTGGAGGAGCAGTGGTGACGGG |
| | | TGTGACAGCAGTAGCCCAGAAGACAGTGGAGGGAGCAGGGAGCA |
| | | TTGCAGCAGCCACTGGCTTTGTCAAAAAGGACCAGTTGGGCAAGA |
| | | ATGAAGAAGGAGCCCCACAGGAAGGAATTCTGGAAGATATGCCT |
| | | GTGGATCCTGACAATGAGGCTTATGAAATGCCTTCTGAGGAAGGG |
| | | TATCAAGACTACGAACCTGAAGCCTAAGAAATATCTTTGCTCCCA |
| | | GTTTCTTGAGATCTGCTGACAGATGTTCCATCCTGTACAAGTGCTC |
| | | AGTTCCAATGTGCCCAGTCATGACATTTCTCAAAGTTTTTACAGTG |
| | | TATCTCGAAGTCTTCCATCAGCAGTGATTGAAGTATCTGTACCTGC |
| | | CCCCACTCAGCATTTCGGTGCTTCCCTTTCACTGAAGTGAATACAT |
| | | GGTAGCAGGGTCTTTGTGTGCTGTGGATTTTGTGGCTTCAATCTAC |
| | | GATGTTAAAACAAATTAAAAACACCTAAGTGACTACCACTTATTT |
| | | CTAAATCCTCACTATTTTTTTGTTGCTGTTGTTCAGAAGTTGTTAG |
| | | TGATTTGCTATCATATATTATAAGATTTTTAGGTGTCTTTTAATGA |
| | | TACTGTCTAAGAATAATGACGTATTGTGAAATTTGTTAATATATAT |
| | | AATACTTAAAAATATGTGAGCATGAAACTATGCACCTATAAATAC |
| | | TAAATATGAAATTTTACCATTTTGCGATGTGTTTTATTCACTTGTG |
| | | TTTGTATATAAATGGTGAGAATTAAAATAAAACGTTATCTCATTG |
| | | CAAAAATATTTTATTTTTATCCCATCTCACTTTAATAATAAAAATC |
| | | ATGCTTATAAGCAACATGAATTAAGAACTGACACAAAGGACAAA |
| | | AATATAAAGTTATTAATAGCCATTTGAAGAAGGAGGAATTTTAGA |
| | | AGAGGTAGAGAAAATGGAACATTAACCCTACACTCGGAATTCCCT |
| | | GAAGCAACACTGCCAGAAGTGTGTTTTGGTATGCACTGGTTCCTT |
| | | AAGTGGCTGTGATTAATTATTGAAAGTGGGGTGTTGAAGACCCCA |
| | | ACTACTATTGTAGAGTGGTCTATTTCTCCCTTCAATCCTGTCAATG |
| | | TTTGCTTTACGTATTTTGGGGAACTGTTGTTTGATGTGTATGTGTT |
| | | TATAATTGTTATACATTTTTAATTGAGCCTTTTATTAACATATATT |
| | | GTTATTTTTGTCTCGAAATAATTTTTTAGTTAAAATCTATTTTGTCT |
| | | GATATTGGTGTGAATGCTGTACCTTTCTGACAATAAATAATATTC |
| | | GACCATGAATAAAAAAAAAAAAAAAGTGGGTTCCCGGGAACTAA |
| | | GCAGTGTAGAAGATGATTTTGACTACACCCTCCTTAGAGAGCCAT |
| | | AAGACACATTAGCACATATTAGCACATTCAAGGCTCTGAGAGAAT |
| | | GTGGTTAACTTTGTTTAACTCAGCATTCCTCACTTTTTTTTTTTAAT |
| | | CATCAGAAATTCTCTCTCTCTCTCTCTCTTTTTCTCTCGCTCTCTTT |
| | | TTTTTTTTTTTTTTACAGGAAATGCCTTTAAACATCGTTGGAACTA |
| | | CCAGAGTCACCTTAAAGGAGATCAATTCTCTAGACTGATAAAAAT |
| | | TTCATGGCCTCCTTTAAATGTTGCCAAATATATGAATTCTAGGATT |
| | | TTTCCTTAGGAAAGGTTTTTCTCTTTCAGGGAAGATCTATTAACTC |
| | | CCCATGGGTGCTGAAAATAAACTTGATGGTGAAAAACTCTGTATA |
| | | AATTAATTTAAAAATTATTTGGTTTCTCTTTTTAATTATTCTGGGG |
| | | CATAGTCATTTCTAAAAGTCACTAGTAGAAAGTATAATTTCAAGA |
| | | CAGAATATTCTAGACATGCTAGCAGTTTATATGTATTCATGAGTA |
| | | ATGTGATATATATTGGGCGCTGGTGAGGAAGGAAGGAGGAATGA |
| | | GTGACTATAAGGATGGTTACCATAGAAACTTCCTTTTTTACCTAAT |
| | | TGAAGAGAGACTACTACAGAGTGCTAAGCTGCATGTGTCATCTTA |
| | | CACTAGAGAGAAATGGTAAGTTTCTTGTTTTATTTAAGTTATGTTT |
| | | AAGCAAGGAAAGGATTTGTTATTGAACAGTATATTTCAGGAAGGT |
| | | TAGAAAGTGGCGGTTAGGATATATTTTAAATCTACCTAAAGCAGC |
| | | ATATTTTAAAAATTTAAAAGTATTGGTATTAAATTAAGAAATAGA |
| | | GGACAGAACTAGACTGATAGCAGTGACCTAGAACAATTTGAGAT |
| | | TAGGAAAGTTGTGACCATGAATTTAAGGATTTATGTGGATACAAA |
| | | TTCTCCTTTAAAGTGTTTCTTCCCTTAATATTTATCTGACGGTAATT |
| | | TTTGAGCAGTGAATTACTTTATATATCTTAATAGTTTATTTGGGAC |
| | | CAAACACTTAAACAAAAAGTTCTTTAAGTCATATAAGCCTTTTCA |
| | | GGAAGCTTGTCTCATATTCACTCCCGAGACATTCACCTGCCAAGT |
| | | GGCCTGAGGATCAATCCAGTCCTAGGTTTATTTTGCAGACTTACA |
| | | TTCTCCCAAGTTATTCAGCCTCATATGACTCCACGGTCGGCTTTAC |
| | | CAAAACAGTTCAGAGTGCACTTTGGCACACAATTGGGAACAGAA |
| | | CAATCTAATGTGTGGTTTGGTATTCCAAGTGGGGTCTTTTTCAGAA |
| | | TCTCTGCACTAGTGTGAGATGCAAACATGTTTCCTCATCTTTCTGG |
| | | CTTATCCAGTATGTAGCTATTTGTGACATAATAAATATATACATAT |
| | | ATGAAAATA |
| 31 | Variant | GGCGACGACCAGAAGGGGCCCAAGAGAGGGGGCGAGCGACCGA |
| | 7 | GCGCCGCGACGCGGAAGTGAGGTGCGTGCGGGCTGCAGCGCAGA |
| | | CCCCGGCCCGGCCCCTCCGAGAGCGTCCTGGGCGCTCCCTCACGC |
| | | CTTGCCTTCAAGCCTTCTGCCTTTCCACCCTCGTGAGCGGAGAACT |
| | | GGGAGTGGCCATTCGACGACAGGTTAGCGGGTTTGCCTCCCACTC |
| | | CCCCAGCCTCGCGTCGCCGGCTCACAGCGGCCTCCTCTGGGGACA |
| | | GTCCCCCCCGGGTGCCGCCTCCGCCCTTCCTGTGCGCTCCTTTTCC |
| | | TTCTTCTTTCCTATTAAATATTATTTGGGAATTGTTTAAATTTTTTT |
| | | TTTAAAAAAAGAGAGAGGCGGGGAGGAGTCGGAGTTGTGGAGAA |
| | | GCAGAGGGACTCAGGGCTGAGAGATTAGGCTGCTTCTCCGGGATC |
| | | CGCTTTTCCCCGGGAAACGCGAGGATGCTCCATGGAGCTGTGGTG |
| | | TAAAGGAATTCATTAGCCATGGATGTATTCATGAAAGGACTTTCA |
| | | AAGGCCAAGGAGGGAGTTGTGGCTGCTGCTGAGAAAACCAAACA |
| | | GGGTGTGGCAGAAGCAGCAGGAAAGACAAAAGAGGGTGTTCTCT |
| | | ATGTAGGCTCCAAAACCAAGGAGGGAGTGGTGCATGGTGTGGCA |
| | | ACAGTGGCTGAGAAGACCAAAGAGCAAGTGACAAATGTTGGAGG |
| | | AGCAGTGGTGACGGGTGTGACAGCAGTAGCCCAGAAGACAGTGG |
| | | AGGGAGCAGGGAGCATTGCAGCAGCCACTGGCTTTGTCAAAAAG |
| | | GACCAGTTGGGCAAGAATGAAGAAGGAGCCCCACAGGAAGGAAT |
| | | TCTGGAAGATATGCCTGTGGATCCTGACAATGAGGCTTATGAAAT |
| | | GCCTTCTGAGGAAGGGTATCAAGACTACGAACCTGAAGCCTAAG |
| | | AAATATCTTTGCTCCCAGTTTCTTGAGATCTGCTGACAGATGTTCC |
| | | ATCCTGTACAAGTGCTCAGTTCCAATGTGCCCAGTCATGACATTTC |
| | | TCAAAGTTTTTACAGTGTATCTCGAAGTCTTCCATCAGCAGTGATT |
| | | GAAGTATCTGTACCTGCCCCCACTCAGCATTTCGGTGCTTCCCTTT |
| | | CACTGAAGTGAATACATGGTAGCAGGGTCTTTGTGTGCTGTGGAT |
| | | TTTGTGGCTTCAATCTACGATGTTAAAACAAATTAAAAACACCTA |
| | | AGTGACTACCACTTATTTCTAAATCCTCACTATTTTTTTGTTGCTGT |
| | | TGTTCAGAAGTTGTTAGTGATTTGCTATCATATATTATAAGATTTT |
| | | TAGGTGTCTTTTAATGATACTGTCTAAGAATAATGACGTATTGTG |
| | | AAATTTGTTAATATATATAATACTTAAAAATATGTGAGCATGAAA |
| | | CTATGCACCTATAAATACTAAATATGAAATTTTACCATTTTGCGAT |
| | | GTGTTTTATTCACTTGTGTTTGTATATAAATGGTGAGAATTAAAAT |
| | | AAAACGTTATCTCATTGCAAAAATATTTTATTTTTATCCCATCTCA |
| | | CTTTAATAATAAAAATCATGCTTATAAGCAACATGAATTAAGAAC |
| | | TGACACAAAGGACAAAAATATAAAGTTATTAATAGCCATTTGAA |
| | | GAAGGAGGAATTTTAGAAGAGGTAGAGAAAATGGAACATTAACC |
| | | CTACACTCGGAATTCCCTGAAGCAACACTGCCAGAAGTGTGTTTT |
| | | GGTATGCACTGGTTCCTTAAGTGGCTGTGATTAATTATTGAAAGT |
| | | GGGGTGTTGAAGACCCCAACTACTATTGTAGAGTGGTCTATTTCT |
| | | CCCTTCAATCCTGTCAATGTTTGCTTTACGTATTTTGGGGAACTGT |
| | | TGTTTGATGTGTATGTGTTTATAATTGTTATACATTTTTAATTGAG |
| | | CCTTTTATTAACATATATTGTTATTTTTGTCTCGAAATAATTTTTTA |
| | | GTTAAAATCTATTTTGTCTGATATTGGTGTGAATGCTGTACCTTTC |
| | | TGACAATAAATAATATTCGACCATGAATAAAAAAAAAAAAAAAG |
| | | TGGGTTCCCGGGAACTAAGCAGTGTAGAAGATGATTTTGACTACA |
| | | CCCTCCTTAGAGAGCCATAAGACACATTAGCACATATTAGCACAT |
| | | TCAAGGCTCTGAGAGAATGTGGTTAACTTTGTTTAACTCAGCATT |
| | | CCTCACTTTTTTTTTTTAATCATCAGAAATTCTCTCTCTCTCTCTCT |
| | | CTTTTTCTCTCGCTCTCTTTTTTTTTTTTTTTTTACAGGAAATGCCTT |
| | | TAAACATCGTTGGAACTACCAGAGTCACCTTAAAGGAGATCAATT |
| | | CTCTAGACTGATAAAAATTTCATGGCCTCCTTTAAATGTTGCCAA |
| | | ATATATGAATTCTAGGATTTTTCCTTAGGAAAGGTTTTTCTCTTTC |
| | | AGGGAAGATCTATTAACTCCCCATGGGTGCTGAAAATAAACTTGA |
| | | TGGTGAAAAACTCTGTATAAATTAATTTAAAAATTATTTGGTTTCT |
| | | CTTTTTAATTATTCTGGGGCATAGTCATTTCTAAAAGTCACTAGTA |
| | | GAAAGTATAATTTCAAGACAGAATATTCTAGACATGCTAGCAGTT |
| | | TATATGTATTCATGAGTAATGTGATATATATTGGGCGCTGGTGAG |
| | | GAAGGAAGGAGGAATGAGTGACTATAAGGATGGTTACCATAGAA |
| | | ACTTCCTTTTTTACCTAATTGAAGAGAGACTACTACAGAGTGCTA |
| | | AGCTGCATGTGTCATCTTACACTAGAGAGAAATGGTAAGTTTCTT |
| | | GTTTTATTTAAGTTATGTTTAAGCAAGGAAAGGATTTGTTATTGAA |
| | | CAGTATATTTCAGGAAGGTTAGAAAGTGGCGGTTAGGATATATTT |
| | | TAAATCTACCTAAAGCAGCATATTTTAAAAATTTAAAAGTATTGG |
| | | TATTAAATTAAGAAATAGAGGACAGAACTAGACTGATAGCAGTG |
| | | ACCTAGAACAATTTGAGATTAGGAAAGTTGTGACCATGAATTTAA |
| | | GGATTTATGTGGATACAAATTCTCCTTTAAAGTGTTTCTTCCCTTA |
| | | ATATTTATCTGACGGTAATTTTTGAGCAGTGAATTACTTTATATAT |
| | | CTTAATAGTTTATTTGGGACCAAACACTTAAACAAAAAGTTCTTT |
| | | AAGTCATATAAGCCTTTTCAGGAAGCTTGTCTCATATTCACTCCCG |
| | | AGACATTCACCTGCCAAGTGGCCTGAGGATCAATCCAGTCCTAGG |
| | | TTTATTTTGCAGACTTACATTCTCCCAAGTTATTCAGCCTCATATG |
| | | ACTCCACGGTCGGCTTTACCAAAACAGTTCAGAGTGCACTTTGGC |
| | | ACACAATTGGGAACAGAACAATCTAATGTGTGGTTTGGTATTCCA |
| | | AGTGGGGTCTTTTTCAGAATCTCTGCACTAGTGTGAGATGCAAAC |
| | | ATGTTTCCTCATCTTTCTGGCTTATCCAGTATGTAGCTATTTGTGA |
| | | CATAATAAATATATACATATATGAAAATA |
| 32 | Variant | GAGTGTGAGCGGCGCCTGCTCAGGGTAGATAGCTGAGGGCGGGG |
| | 8 | GTGGATGTTGGATGGATTAGAACCATCACACTTGGGCCTGCTGTT |
| | | TGCCTGAGTTTGAACCACACCCCGATGTGGTGTAAAGGAATTCAT |
| | | TAGCCATGGATGTATTCATGAAAGGACTTTCAAAGGCCAAGGAG |
| | | GGAGTTGTGGCTGCTGCTGAGAAAACCAAACAGGGTGTGGCAGA |
| | | AGCAGCAGGAAAGACAAAAGAGGGTGTTCTCTATGTAGGCTCCA |
| | | AAACCAAGGAGGGAGTGGTGCATGGTGTGGCAACAGTGGCTGAG |
| | | AAGACCAAAGAGCAAGTGACAAATGTTGGAGGAGCAGTGGTGAC |
| | | GGGTGTGACAGCAGTAGCCCAGAAGACAGTGGAGGGAGCAGGGA |
| | | GCATTGCAGCAGCCACTGGCTTTGTCAAAAAGGACCAGTTGGGCA |
| | | AGAATGAAGAAGGAGCCCCACAGGAAGGAATTCTGGAAGATATG |
| | | CCTGTGGATCCTGACAATGAGGCTTATGAAATGCCTTCTGAGGAA |
| | | GGGTATCAAGACTACGAACCTGAAGCCTAAGAAATATCTTTGCTC |
| | | CCAGTTTCTTGAGATCTGCTGACAGATGTTCCATCCTGTACAAGTG |
| | | CTCAGTTCCAATGTGCCCAGTCATGACATTTCTCAAAGTTTTTACA |
| | | GTGTATCTCGAAGTCTTCCATCAGCAGTGATTGAAGTATCTGTAC |
| | | CTGCCCCCACTCAGCATTTCGGTGCTTCCCTTTCACTGAAGTGAAT |
| | | ACATGGTAGCAGGGTCTTTGTGTGCTGTGGATTTTGTGGCTTCAAT |
| | | CTACGATGTTAAAACAAATTAAAAACACCTAAGTGACTACCACTT |
| | | ATTTCTAAATCCTCACTATTTTTTTGTTGCTGTTGTTCAGAAGTTGT |
| | | TAGTGATTTGCTATCATATATTATAAGATTTTTAGGTGTCTTTTAA |
| | | TGATACTGTCTAAGAATAATGACGTATTGTGAAATTTGTTAATAT |
| | | ATATAATACTTAAAAATATGTGAGCATGAAACTATGCACCTATAA |
| | | ATACTAAATATGAAATTTTACCATTTTGCGATGTGTTTTATTCACT |
| | | TGTGTTTGTATATAAATGGTGAGAATTAAAATAAAACGTTATCTC |
| | | ATTGCAAAAATATTTTATTTTTATCCCATCTCACTTTAATAATAAA |
| | | AATCATGCTTATAAGCAACATGAATTAAGAACTGACACAAAGGA |
| | | CAAAAATATAAAGTTATTAATAGCCATTTGAAGAAGGAGGAATTT |
| | | TAGAAGAGGTAGAGAAAATGGAACATTAACCCTACACTCGGAAT |
| | | TCCCTGAAGCAACACTGCCAGAAGTGTGTTTTGGTATGCACTGGT |
| | | TCCTTAAGTGGCTGTGATTAATTATTGAAAGTGGGGTGTTGAAGA |
| | | CCCCAACTACTATTGTAGAGTGGTCTATTTCTCCCTTCAATCCTGT |
| | | CAATGTTTGCTTTACGTATTTTGGGGAACTGTTGTTTGATGTGTAT |
| | | GTGTTTATAATTGTTATACATTTTTAATTGAGCCTTTTATTAACAT |
| | | ATATTGTTATTTTTGTCTCGAAATAATTTTTTAGTTAAAATCTATTT |
| | | TGTCTGATATTGGTGTGAATGCTGTACCTTTCTGACAATAAATAAT |
| | | ATTCGACCATGAATAAAAAAAAAAAAAAAGTGGGTTCCCGGGAA |
| | | CTAAGCAGTGTAGAAGATGATTTTGACTACACCCTCCTTAGAGAG |
| | | CCATAAGACACATTAGCACATATTAGCACATTCAAGGCTCTGAGA |
| | | GAATGTGGTTAACTTTGTTTAACTCAGCATTCCTCACTTTTTTTTTT |
| | | TAATCATCAGAAATTCTCTCTCTCTCTCTCTCTTTTTCTCTCGCTCT |
| | | CTTTTTTTTTTTTTTTTTACAGGAAATGCCTTTAAACATCGTTGGAA |
| | | CTACCAGAGTCACCTTAAAGGAGATCAATTCTCTAGACTGATAAA |
| | | AATTTCATGGCCTCCTTTAAATGTTGCCAAATATATGAATTCTAGG |
| | | ATTTTTCCTTAGGAAAGGTTTTTCTCTTTCAGGGAAGATCTATTAA |
| | | CTCCCCATGGGTGCTGAAAATAAACTTGATGGTGAAAAACTCTGT |
| | | ATAAATTAATTTAAAAATTATTTGGTTTCTCTTTTTAATTATTCTG |
| | | GGGCATAGTCATTTCTAAAAGTCACTAGTAGAAAGTATAATTTCA |
| | | AGACAGAATATTCTAGACATGCTAGCAGTTTATATGTATTCATGA |
| | | GTAATGTGATATATATTGGGCGCTGGTGAGGAAGGAAGGAGGAA |
| | | TGAGTGACTATAAGGATGGTTACCATAGAAACTTCCTTTTTTACCT |
| | | AATTGAAGAGAGACTACTACAGAGTGCTAAGCTGCATGTGTCATC |
| | | TTACACTAGAGAGAAATGGTAAGTTTCTTGTTTTATTTAAGTTATG |
| | | TTTAAGCAAGGAAAGGATTTGTTATTGAACAGTATATTTCAGGAA |
| | | GGTTAGAAAGTGGCGGTTAGGATATATTTTAAATCTACCTAAAGC |
| | | AGCATATTTTAAAAATTTAAAAGTATTGGTATTAAATTAAGAAAT |
| | | AGAGGACAGAACTAGACTGATAGCAGTGACCTAGAACAATTTGA |
| | | GATTAGGAAAGTTGTGACCATGAATTTAAGGATTTATGTGGATAC |
| | | AAATTCTCCTTTAAAGTGTTTCTTCCCTTAATATTTATCTGACGGT |
| | | AATTTTTGAGCAGTGAATTACTTTATATATCTTAATAGTTTATTTG |
| | | GGACCAAACACTTAAACAAAAAGTTCTTTAAGTCATATAAGCCTT |
| | | TTCAGGAAGCTTGTCTCATATTCACTCCCGAGACATTCACCTGCCA |
| | | AGTGGCCTGAGGATCAATCCAGTCCTAGGTTTATTTTGCAGACTT |
| | | ACATTCTCCCAAGTTATTCAGCCTCATATGACTCCACGGTCGGCTT |
| | | TACCAAAACAGTTCAGAGTGCACTTTGGCACACAATTGGGAACAG |
| | | AACAATCTAATGTGTGGTTTGGTATTCCAAGTGGGGTCTTTTTCAG |
| | | AATCTCTGCACTAGTGTGAGATGCAAACATGTTTCCTCATCTTTCT |
| | | GGCTTATCCAGTATGTAGCTATTTGTGACATAATAAATATATACA |
| | | TATATGAAAATA |
| 33 | Variant | GCTCTAATTCTCTGCACCTTCTCAAGCATTGTGCAGATTGGTTTTC |
| | 9 | TGGATTATCAGCCTGAAGGACAAAACGAAGAAACAGCCATTAGC |
| | | TCCTGTCTCCCATTGTCTGAGAGCTGCCACTAGGATATTAACTTCC |
| | | TGAAATTCTGCAGAAATCTCCTCTTACTTTGGCACTGGAGATGCC |
| | | CATACGCAGAAAGCAAAAAGGCACAGCATATTTAAGGAAGCTCA |
| | | TAAGAAACAGTGCATCCAGAAGTGGCGAGAATTGGAGGAATGGA |
| | | CATGAGACTCTAAGAACCAGCGCCTTTGATGTTCCTTTTGATCTGT |
| | | TATGTAGCTCTTCTTGTACACAGAATGAAGAAGGAGCCCCACAGG |
| | | AAGGAATTCTGGAAGATATGCCTGTGGATCCTGACAATGAGGCTT |
| | | ATGAAATGCCTTCTGAGGAAGGGTATCAAGACTACGAACCTGAA |
| | | GCCTAAGAAATATCTTTGCTCCCAGTTTCTTGAGATCTGCTGACAG |
| | | ATGTTCCATCCTGTACAAGTGCTCAGTTCCAATGTGCCCAGTCATG |
| | | ACATTTCTCAAAGTTTTTACAGTGTATCTCGAAGTCTTCCATCAGC |
| | | AGTGATTGAAGTATCTGTACCTGCCCCCACTCAGCATTTCGGTGCT |
| | | TCCCTTTCACTGAAGTGAATACATGGTAGCAGGGTCTTTGTGTGCT |
| | | GTGGATTTTGTGGCTTCAATCTACGATGTTAAAACAAATTAAAAA |
| | | CACCTAAGTGACTACCACTTATTTCTAAATCCTCACTATTTTTTTG |
| | | TTGCTGTTGTTCAGAAGTTGTTAGTGATTTGCTATCATATATTATA |
| | | AGATTTTTAGGTGTCTTTTAATGATACTGTCTAAGAATAATGACGT |
| | | ATTGTGAAATTTGTTAATATATATAATACTTAAAAATATGTGAGC |
| | | ATGAAACTATGCACCTATAAATACTAAATATGAAATTTTACCATT |
| | | TTGCGATGTGTTTTATTCACTTGTGTTTGTATATAAATGGTGAGAA |
| | | TTAAAATAAAACGTTATCTCATTGCAAAAATATTTTATTTTTATCC |
| | | CATCTCACTTTAATAATAAAAATCATGCTTATAAGCAACATGAAT |
| | | TAAGAACTGACACAAAGGACAAAAATATAAAGTTATTAATAGCC |
| | | ATTTGAAGAAGGAGGAATTTTAGAAGAGGTAGAGAAAATGGAAC |
| | | ATTAACCCTACACTCGGAATTCCCTGAAGCAACACTGCCAGAAGT |
| | | GTGTTTTGGTATGCACTGGTTCCTTAAGTGGCTGTGATTAATTATT |
| | | GAAAGTGGGGTGTTGAAGACCCCAACTACTATTGTAGAGTGGTCT |
| | | ATTTCTCCCTTCAATCCTGTCAATGTTTGCTTTACGTATTTTGGGG |
| | | AACTGTTGTTTGATGTGTATGTGTTTATAATTGTTATACATTTTTA |
| | | ATTGAGCCTTTTATTAACATATATTGTTATTTTTGTCTCGAAATAA |
| | | TTTTTTAGTTAAAATCTATTTTGTCTGATATTGGTGTGAATGCTGT |
| | | ACCTTTCTGACAATAAATAATATTCGACCATGAATAAAAAAAAAA |
| | | AAAAAGTGGGTTCCCGGGAACTAAGCAGTGTAGAAGATGATTTT |
| | | GACTACACCCTCCTTAGAGAGCCATAAGACACATTAGCACATATT |
| | | AGCACATTCAAGGCTCTGAGAGAATGTGGTTAACTTTGTTTAACT |
| | | CAGCATTCCTCACTTTTTTTTTTTAATCATCAGAAATTCTCTCTCTC |
| | | TCTCTCTCTTTTTCTCTCGCTCTCTTTTTTTTTTTTTTTTTACAGGAA |
| | | ATGCCTTTAAACATCGTTGGAACTACCAGAGTCACCTTAAAGGAG |
| | | ATCAATTCTCTAGACTGATAAAAATTTCATGGCCTCCTTTAAATGT |
| | | TGCCAAATATATGAATTCTAGGATTTTTCCTTAGGAAAGGTTTTTC |
| | | TCTTTCAGGGAAGATCTATTAACTCCCCATGGGTGCTGAAAATAA |
| | | ACTTGATGGTGAAAAACTCTGTATAAATTAATTTAAAAATTATTT |
| | | GGTTTCTCTTTTTAATTATTCTGGGGCATAGTCATTTCTAAAAGTC |
| | | ACTAGTAGAAAGTATAATTTCAAGACAGAATATTCTAGACATGCT |
| | | AGCAGTTTATATGTATTCATGAGTAATGTGATATATATTGGGCGC |
| | | TGGTGAGGAAGGAAGGAGGAATGAGTGACTATAAGGATGGTTAC |
| | | CATAGAAACTTCCTTTTTTACCTAATTGAAGAGAGACTACTACAG |
| | | AGTGCTAAGCTGCATGTGTCATCTTACACTAGAGAGAAATGGTAA |
| | | GTTTCTTGTTTTATTTAAGTTATGTTTAAGCAAGGAAAGGATTTGT |
| | | TATTGAACAGTATATTTCAGGAAGGTTAGAAAGTGGCGGTTAGGA |
| | | TATATTTTAAATCTACCTAAAGCAGCATATTTTAAAAATTTAAAA |
| | | GTATTGGTATTAAATTAAGAAATAGAGGACAGAACTAGACTGAT |
| | | AGCAGTGACCTAGAACAATTTGAGATTAGGAAAGTTGTGACCATG |
| | | AATTTAAGGATTTATGTGGATACAAATTCTCCTTTAAAGTGTTTCT |
| | | TCCCTTAATATTTATCTGACGGTAATTTTTGAGCAGTGAATTACTT |
| | | TATATATCTTAATAGTTTATTTGGGACCAAACACTTAAACAAAAA |
| | | GTTCTTTAAGTCATATAAGCCTTTTCAGGAAGCTTGTCTCATATTC |
| | | ACTCCCGAGACATTCACCTGCCAAGTGGCCTGAGGATCAATCCAG |
| | | TCCTAGGTTTATTTTGCAGACTTACATTCTCCCAAGTTATTCAGCC |
| | | TCATATGACTCCACGGTCGGCTTTACCAAAACAGTTCAGAGTGCA |
| | | CTTTGGCACACAATTGGGAACAGAACAATCTAATGTGTGGTTTGG |
| | | TATTCCAAGTGGGGTCTTTTTCAGAATCTCTGCACTAGTGTGAGAT |
| | | GCAAACATGTTTCCTCATCTTTCTGGCTTATCCAGTATGTAGCTAT |
| | | TTGTGACATAATAAATATATACATATATGAAAATA |

In some cases, a region of Alpha-synuclein polypeptide can be targeted utilizing compositions provided herein. Suitable regions include but are not limited to a N-terminal A2 lipid-binding alpha-helix domain, a Non-amyloid β component (NAC) domain, or a C-terminal acidic domain.

In some cases, a target residue may be located among residues 1-10, 10-20, 20-40, 40-60, 60-80, 80-100, 100-120, or 120-140, overlapping portions thereof, and combinations thereof.

In some aspects, a region from an RNA sequence encoding an alpha-synuclein polypeptide sequence is targeted by an engineered polynucleotide as disclosed herein. Exemplary alpha-synuclein polypeptide sequences encoded by mRNA sequences that are targeted by engineered polynucleotides as disclosed herein are shown in Table 5. Any nucleotide of a polynucleotide sequence encoding a peptide of Table 5 can be targeted by an engineered polynucleotide as disclosed herein. In some cases, a target nucleotide may encode a residue located among residues 1-10, 10-20, 20-40, 40-60, 60-80, 80-100, 100-120, or 120-140, overlapping portions thereof, and combinations thereof, of a peptide of Table 5.

TABLE 5
Human Alpha-synuclein (SNCA) polypeptide
sequences associated with isoform
of Table 4

| SEQ | | |
|---|---|---|
| ID | | |
| NO: | Isoform | SNCA Polypeptide Sequence |
| 34 | Variant | MDVFMKGLSKAKEGVVAAAEKTKQGVAEA |
| | 1-3; | AGKTKEGVLYVGSKTKEGVVHGVATVAEK |
| | 5-8 | TKEQVTNVGGAVVTGVTAVAQKTVEGAGS |
| | | IAAATGFVKKDQLGKNEEGAPQEGILEDM |
| | | PVDPDNEAYEMPSEEGYQDYEPEA |
| 35 | Variant 4 | MDVFMKGLSKAKEGVVAAAEKTKQGVAEA |
| | | AGKTKEGVLYVGSKTKEGVVHGVATVAEK |
| | | TKEQVTNVGGAVVTGVTAVAQKTVEGAGS |
| | | IAAATGFVKKDQLGKEGYQDYEPEA |
| 36 | Variant 9 | MPIRRKQKGTAYLRKLIRNSASRSGENWR |
| | | NGHETLRTSAFDVPFDLLCSSSCTQNEEG |
| | | APQEGILEDMPVDPDNEAYEMPSEEGYQD |
| | | YEPEA |

In some embodiments, the engineered polynucleotide disclosed herein facilitates editing of a nucleotide of a codon that encodes a residue mutation, such as a residue mutation shown in Table 6. In some embodiments, the editing of a nucleotide of a codon that encodes a residue mutation results in a corrected residue upon translation of the edited codon.

Exemplary regions that can be targeted utilizing compositions provided herein can include but are not limited to exon 2 or exon 3. Therefore, an engineered polynucleotide disclosed herein can target a region of a target RNA that comprises a sequence encoding exon 2 or exon 3. In some cases, a target nucleotide of a codon that encodes an amino acid residue of an SNCA polypeptide sequence is any one of amino acid residues: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, and/or 140.

In some embodiments, the engineered polynucleotide disclosed herein facilitates editing of a nucleotide of a codon that encodes an amino acid residue mutation, such as an amino acid residue at position 30, 46, or 53 of the alpha-synuclein polypeptide of SEQ ID NO: 34 or SEQ ID NO: 35. In other cases, a nucleotide of a codon that encodes an amino acid residue mutation residue can be an amino acid at position 49 (Exon 3) or position 136 (Exon 6), or a nucleotide at position 534 (3′UTR), or 926 (3′UTR). These amino acid residue mutations are listed in Table 6. In some embodiments, the engineered polynucleotide disclosed herein facilitates editing of a nucleotide of a codon that encodes an amino acid residue mutation, such as an amino acid residue mutation shown in Table 6. In some embodiments, the editing of a nucleotide of a codon that encodes an amino acid residue mutation results in a corrected amino acid residue upon translation of the edited codon. In some embodiments, engineered polynucleotide facilitates editing of the translation initiation site (TIS) of the SNCA mRNA (e.g., editing the A of the ATG codon). In some embodiments, the editing of the TIS results in a knockdown of expression the SNCA polypeptide from the edited SNCA mRNA.

TABLE 6
SNCA exons associated with provided missense,
nonsense, and frameshift mutations from relevant
polypeptide sequences in Table 5.

| Region of SNCA | Protein Mutation |
|---|---|
| Exon 2 | A30P |
| Exon 3 | E46K; A53T |

### Tau

Tau proteins (Tau-p) are encoded by six mRNA isoforms of Tau MAPT. Tau-p is a microtubule-binding protein, important for microtubule stability and transport. It is primarily expressed in the neurons of the CNS. The aggregation of hyperphosphorylated mutant Tau proteins into neurofibrillary tangles (NFTs) in the human brain causes a group of neurodegenerative diseases named Taupathies, including Parkinson's Disease, Alzheimer's, Frontotemporal Dementia (FTD), Chronic Traumatic Encephalopathy (CTE), Progressive Supranuclear Palsy, and Corticobasal Degeneration. Tau proteins can also be associated with Alzheimer's disease, Proteolytic Tau cleavage fragments can also be directly neurotoxic. Therefore, a multiplex strategy to substantially reduce Tau formation can be important in effectively treating neurodegenerative diseases.

In an embodiment, a specific nucleotide can be targeted utilizing compositions and methods provided herein. Exemplary Tau mRNA sequences are shown in Table 7. In some cases, a target nucleotide can be located at any position of a target sequence. In some cases, a target nucleotide may be located among nucleotide residues 1-100, 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, 1901-2000, 2001-2100, 2101-2200, 2201-2300, 2301-2400, 2401-2500, 2501-2600, 2601-2700, 2701-2800, 2801-2900, 2901-3000, 3001-3100, 3101-3200, 3201-3300, 3301-3400, 3401-3500, 3501-3600, 3601-3700, 3701-3800, 3801-3900, 3901-4000, 4001-4100, 4101-4200, 4201-4300, 4301-4400, 4401-4500, 4501-4600, 4601-4700, 4701-4800, 4801-4900, 4901-5000, 5001-5100, 5101-5200, 5201-5300, 5301-5400, 5401-5500, 5501-5600, 5601-5700, 5701-5800, 5801-5900, 5901-6000, 6001-6100, 6101-6200, 6201-6300, 6301-6400, 6401-6500, 6501-6600, and/or 6601-6644, or any combination thereof of the Tau mRNA. In some embodiments, engineered polynucleotide facilitates editing of the translation initiation site (TIS) of the MAPT mRNA (e.g., editing the A of the ATG codon). In some embodiments, the editing of the TIS results in a knockdown of expression the Tau polypeptide from the edited MAPT mRNA.

TABLE 7
Human MAPT mRNA Isoform Sequences. Sequences obtained from NCBI MAPT
gene ID: 4137; Assembly GRCh38.p13 (GCF 000001405.39); NC_000017.11
(45894538 . . . 46028334)

| SEQ | | |
|---|---|---|
| ID NO: | Isoform | mRNA sequences |
| 37 | 1 | GCAGTCACCGCCACCCACCAGCTCCGGCACCAACAGCAGCGCCG |
| | | CTGCCACCGCCCACCTTCTGCCGCCGCCACCACAGCCACCTTCTCC |
| | | TCCTCCGCTGTCCTCTCCCGTCCTCGCCTCTGTCGACTATCAGGTG |
| | | AACTTTGAACCAGGATGGCTGAGCCCCGCCAGGAGTTCGAAGTG |
| | | ATGGAAGATCACGCTGGGACGTACGGGTTGGGGGACAGGAAAGA |
| | | TCAGGGGGGCTACACCATGCACCAAGACCAAGAGGGTGACACGG |
| | | ACGCTGGCCTGAAAGAATCTCCCCTGCAGACCCCCACTGAGGACG |
| | | GATCTGAGGAACCGGGCTCTGAAACCTCTGATGCTAAGAGCACTC |
| | | CAACAGCGGAAGATGTGACAGCACCCTTAGTGGATGAGGGAGCT |
| | | CCCGGCAAGCAGGCTGCCGCGCAGCCCCACACGGAGATCCCAGA |
| | | AGGAACCACAGCTGAAGAAGCAGGCATTGGAGACACCCCCAGCC |
| | | TGGAAGACGAAGCTGCTGGTCACGTGACCCAAGAGCCTGAAAGT |
| | | GGTAAGGTGGTCCAGGAAGGCTTCCTCCGAGAGCCAGGCCCCCC |
| | | AGGTCTGAGCCACCAGCTCATGTCCGGCATGCCTGGGGCTCCCCT |
| | | CCTGCCTGAGGGCCCCAGAGAGGCCACACGCCAACCTTCGGGGA |
| | | CAGGACCTGAGGACACAGAGGGCGGCCGCCACGCCCCTGAGCTG |
| | | CTCAAGCACCAGCTTCTAGGAGACCTGCACCAGGAGGGGCCGCC |
| | | GCTGAAGGGGGCAGGGGGCAAAGAGAGGCCGGGGAGCAAGGAG |
| | | GAGGTGGATGAAGACCGCGACGTCGATGAGTCCTCCCCCCAAGA |
| | | CTCCCCTCCCTCCAAGGCCTCCCCAGCCCAAGATGGGCGGCCTCC |
| | | CCAGACAGCCGCCAGAGAAGCCACCAGCATCCCAGGCTTCCCAG |
| | | CGGAGGGTGCCATCCCCCTCCCTGTGGATTTCCTCTCCAAAGTTTC |
| | | CACAGAGATCCCAGCCTCAGAGCCCGACGGGCCCAGTGTAGGGC |
| | | GGGCCAAAGGGCAGGATGCCCCCCTGGAGTTCACGTTTCACGTGG |
| | | AAATCACACCCAACGTGCAGAAGGAGCAGGCGCACTCGGAGGAG |
| | | CATTTGGGAAGGGCTGCATTTCCAGGGGCCCCTGGAGAGGGGCC |
| | | AGAGGCCCGGGGCCCCTCTTTGGGAGAGGACACAAAAGAGGCTG |
| | | ACCTTCCAGAGCCCTCTGAAAAGCAGCCTGCTGCTGCTCCGCGGG |
| | | GGAAGCCCGTCAGCCGGGTCCCTCAACTCAAAGCTCGCATGGTCA |
| | | GTAAAAGCAAAGACGGGACTGGAAGCGATGACAAAAAAGCCAA |
| | | GACATCCACACGTTCCTCTGCTAAAACCTTGAAAAATAGGCCTTG |
| | | CCTTAGCCCCAAACACCCCACTCCTGGTAGCTCAGACCCTCTGAT |
| | | CCAACCCTCCAGCCCTGCTGTGTGCCCAGAGCCACCTTCCTCTCCT |
| | | AAATACGTCTCTTCTGTCACTTCCCGAACTGGCAGTTCTGGAGCA |
| | | AAGGAGATGAAACTCAAGGGGGCTGATGGTAAAACGAAGATCGC |
| | | CACACCGCGGGGAGCAGCCCCTCCAGGCCAGAAGGGCCAGGCCA |
| | | ACGCCACCAGGATTCCAGCAAAAACCCCGCCCGCTCCAAAGACA |
| | | CCACCCAGCTCTGGTGAACCTCCAAAATCAGGGGATCGCAGCGGC |
| | | TACAGCAGCCCCGGCTCCCCAGGCACTCCCGGCAGCCGCTCCCGC |
| | | ACCCCGTCCCTTCCAACCCCACCCACCCGGGAGCCCAAGAAGGTG |
| | | GCAGTGGTCCGTACTCCACCCAAGTCGCCGTCTTCCGCCAAGAGC |
| | | CGCCTGCAGACAGCCCCCGTGCCCATGCCAGACCTGAAGAATGTC |
| | | AAGTCCAAGATCGGCTCCACTGAGAACCTGAAGCACCAGCCGGG |
| | | AGGCGGGAAGGTGCAGATAATTAATAAGAAGCTGGATCTTAGCA |
| | | ACGTCCAGTCCAAGTGTGGCTCAAAGGATAATATCAAACACGTCC |
| | | CGGGAGGCGGCAGTGTGCAAATAGTCTACAAACCAGTTGACCTG |
| | | AGCAAGGTGACCTCCAAGTGTGGCTCATTAGGCAACATCCATCAT |
| | | AAACCAGGAGGTGGCCAGGTGGAAGTAAAATCTGAGAAGCTTGA |
| | | CTTCAAGGACAGAGTCCAGTCGAAGATTGGGTCCCTGGACAATAT |
| | | CACCCACGTCCCTGGCGGAGGAAATAAAAAGATTGAAACCCACA |
| | | AGCTGACCTTCCGCGAGAACGCCAAAGCCAAGACAGACCACGGG |
| | | GCGGAGATCGTGTACAAGTCGCCAGTGGTGTCTGGGGACACGTCT |
| | | CCACGGCATCTCAGCAATGTCTCCTCCACCGGCAGCATCGACATG |
| | | GTAGACTCGCCCCAGCTCGCCACGCTAGCTGACGAGGTGTCTGCC |
| | | TCCCTGGCCAAGCAGGGTTTGTGATCAGGCCCCTGGGGCGGTCAA |
| | | TAATTGTGGAGAGGAGAGAATGAGAGAGTGTGGAAAAAAAAAG |
| | | AATAATGACCCGGCCCCCGCCCTCTGCCCCCAGCTGCTCCTCGCA |
| | | GTTCGGTTAATTGGTTAATCACTTAACCTGCTTTTGTCACTCGGCT |
| | | TTGGCTCGGGACTTCAAAATCAGTGATGGGAGTAAGAGCAAATTT |
| | | CATCTTTCCAAATTGATGGGTGGGCTAGTAATAAAATATTTAAAA |
| | | AAAAACATTCAAAAACATGGCCACATCCAACATTTCCTCAGGCAA |
| | | TTCCTTTTGATTCTTTTTTCTTCCCCCTCCATGTAGAAGAGGGAGA |
| | | AGGAGAGGCTCTGAAAGCTGCTTCTGGGGGATTTCAAGGGACTG |
| | | GGGGTGCCAACCACCTCTGGCCCTGTTGTGGGGGTGTCACAGAGG |
| | | CAGTGGCAGCAACAAAGGATTTGAAACTTGGTGTGTTCGTGGAGC |
| | | CACAGGCAGACGATGTCAACCTTGTGTGAGTGTGACGGGGGTTGG |
| | | GGTGGGGCGGGAGGCCACGGGGGAGGCCGAGGCAGGGGCTGGG |
| | | CAGAGGGGAGAGGAAGCACAAGAAGTGGGAGTGGGAGAGGAAG |
| | | CCACGTGCTGGAGAGTAGACATCCCCCTCCTTGCCGCTGGGAGAG |
| | | CCAAGGCCTATGCCACCTGCAGCGTCTGAGCGGCCGCCTGTCCTT |
| | | GGTGGCCGGGGGTGGGGGCCTGCTGTGGGTCAGTGTGCCACCCTC |
| | | TGCAGGGCAGCCTGTGGGAGAAGGGACAGCGGGTAAAAAGAGA |
| | | AGGCAAGCTGGCAGGAGGGTGGCACTTCGTGGATGACCTCCTTAG |
| | | AAAAGACTGACCTTGATGTCTTGAGAGCGCTGGCCTCTTCCTCCC |
| | | TCCCTGCAGGGTAGGGGGCCTGAGTTGAGGGGCTTCCCTCTGCTC |
| | | CACAGAAACCCTGTTTTATTGAGTTCTGAAGGTTGGAACTGCTGC |
| | | CATGATTTTGGCCACTTTGCAGACCTGGGACTTTAGGGCTAACCA |
| | | GTTCTCTTTGTAAGGACTTGTGCCTCTTGGGAGACGTCCACCCGTT |
| | | TCCAAGCCTGGGCCACTGGCATCTCTGGAGTGTGTGGGGGTCTGG |
| | | GAGGCAGGTCCCGAGCCCCCTGTCCTTCCCACGGCCACTGCAGTC |
| | | ACCCCGTCTGCGCCGCTGTGCTGTTGTCTGCCGTGAGAGCCCAAT |
| | | CACTGCCTATACCCCTCATCACACGTCACAATGTCCCGAATTCCC |
| | | AGCCTCACCACCCCTTCTCAGTAATGACCCTGGTTGGTTGCAGGA |
| | | GGTACCTACTCCATACTGAGGGTGAAATTAAGGGAAGGCAAAGT |
| | | CCAGGCACAAGAGTGGGACCCCAGCCTCTCACTCTCAGTTCCACT |
| | | CATCCAACTGGGACCCTCACCACGAATCTCATGATCTGATTCGGT |
| | | TCCCTGTCTCCTCCTCCCGTCACAGATGTGAGCCAGGGCACTGCTC |
| | | AGCTGTGACCCTAGGTGTTTCTGCCTTGTTGACATGGAGAGAGCC |
| | | CTTTCCCCTGAGAAGGCCTGGCCCCTTCCTGTGCTGAGCCCACAG |
| | | CAGCAGGCTGGGTGTCTTGGTTGTCAGTGGTGGCACCAGGATGGA |
| | | AGGGCAAGGCACCCAGGGCAGGCCCACAGTCCCGCTGTCCCCCA |
| | | CTTGCACCCTAGCTTGTAGCTGCCAACCTCCCAGACAGCCCAGCC |
| | | CGCTGCTCAGCTCCACATGCATAGTATCAGCCCTCCACACCCGAC |
| | | AAAGGGGAACACACCCCCTTGGAAATGGTTCTTTTCCCCCAGTCC |
| | | CAGCTGGAAGCCATGCTGTCTGTTCTGCTGGAGCAGCTGAACATA |
| | | TACATAGATGTTGCCCTGCCCTCCCCATCTGCACCCTGTTGAGTTG |
| | | TAGTTGGATTTGTCTGTTTATGCTTGGATTCACCAGAGTGACTATG |
| | | ATAGTGAAAAGAAAAAAAAAAAAAAAAAAGGACGCATGTATCTT |
| | | GAAATGCTTGTAAAGAGGTTTCTAACCCACCCTCACGAGGTGTCT |
| | | CTCACCCCCACACTGGGACTCGTGTGGCCTGTGTGGTGCCACCCT |
| | | GCTGGGGCCTCCCAAGTTTTGAAAGGCTTTCCTCAGCACCTGGGA |
| | | CCCAACAGAGACCAGCTTCTAGCAGCTAAGGAGGCCGTTCAGCTG |
| | | TGACGAAGGCCTGAAGCACAGGATTAGGACTGAAGCGATGATGT |
| | | CCCCTTCCCTACTTCCCCTTGGGGCTCCCTGTGTCAGGGCACAGAC |
| | | TAGGTCTTGTGGCTGGTCTGGCTTGCGGCGCGAGGATGGTTCTCT |
| | | CTGGTCATAGCCCGAAGTCTCATGGCAGTCCCAAAGGAGGCTTAC |
| | | AACTCCTGCATCACAAGAAAAAGGAAGCCACTGCCAGCTGGGGG |
| | | GATCTGCAGCTCCCAGAAGCTCCGTGAGCCTCAGCCACCCCTCAG |
| | | ACTGGGTTCCTCTCCAAGCTCGCCCTCTGGAGGGGCAGCGCAGCC |
| | | TCCCACCAAGGGCCCTGCGACCACAGCAGGGATTGGGATGAATT |
| | | GCCTGTCCTGGATCTGCTCTAGAGGCCCAAGCTGCCTGCCTGAGG |
| | | AAGGATGACTTGACAAGTCAGGAGACACTGTTCCCAAAGCCTTGA |
| | | CCAGAGCACCTCAGCCCGCTGACCTTGCACAAACTCCATCTGCTG |
| | | CCATGAGAAAAGGGAAGCCGCCTTTGCAAAACATTGCTGCCTAA |
| | | AGAAACTCAGCAGCCTCAGGCCCAATTCTGCCACTTCTGGTTTGG |
| | | GTACAGTTAAAGGCAACCCTGAGGGACTTGGCAGTAGAAATCCA |
| | | GGGCCTCCCCTGGGGCTGGCAGCTTCGTGTGCAGCTAGAGCTTTA |
| | | CCTGAAAGGAAGTCTCTGGGCCCAGAACTCTCCACCAAGAGCCTC |
| | | CCTGCCGTTCGCTGAGTCCCAGCAATTCTCCTAAGTTGAAGGGAT |
| | | CTGAGAAGGAGAAGGAAATGTGGGGTAGATTTGGTGGTGGTTAG |
| | | AGATATGCCCCCCTCATTACTGCCAACAGTTTCGGCTGCATTTCTT |
| | | CACGCACCTCGGTTCCTCTTCCTGAAGTTCTTGTGCCCTGCTCTTC |
| | | AGCACCATGGGCCTTCTTATACGGAAGGCTCTGGGATCTCCCCCT |
| | | TGTGGGGCAGGCTCTTGGGGCCAGCCTAAGATCATGGTTTAGGGT |
| | | GATCAGTGCTGGCAGATAAATTGAAAAGGCACGCTGGCTTGTGAT |
| | | CTTAAATGAGGACAATCCCCCCAGGGCTGGGCACTCCTCCCCTCC |
| | | CCTCACTTCTCCCACCTGCAGAGCCAGTGTCCTTGGGTGGGCTAG |
| | | ATAGGATATACTGTATGCCGGCTCCTTCAAGCTGCTGACTCACTTT |
| | | ATCAATAGTTCCATTTAAATTGACTTCAGTGGTGAGACTGTATCCT |
| | | GTTTGCTATTGCTTGTTGTGCTATGGGGGGAGGGGGGAGGAATGT |
| | | GTAAGATAGTTAACATGGGCAAAGGGAGATCTTGGGGTGCAGCA |
| | | CTTAAACTGCCTCGTAACCCTTTTCATGATTTCAACCACATTTGCT |
| | | AGAGGGAGGGAGCAGCCACGGAGTTAGAGGCCCTTGGGGTTTCT |
| | | CTTTTCCACTGACAGGCTTTCCCAGGCAGCTGGCTAGTTCATTCCC |
| | | TCCCCAGCCAGGTGCAGGCGTAGGAATATGGACATCTGGTTGCTT |
| | | TGGCCTGCTGCCCTCTTTCAGGGGTCCTAAGCCCACAATCATGCCT |
| | | CCCTAAGACCTTGGCATCCTTCCCTCTAAGCCGTTGGCACCTCTGT |
| | | GCCACCTCTCACACTGGCTCCAGACACACAGCCTGTGCTTTTGGA |
| | | GCTGAGATCACTCGCTTCACCCTCCTCATCTTTGTTCTCCAAGTAA |
| | | AGCCACGAGGTCGGGGCGAGGGCAGAGGTGATCACCTGCGTGTC |
| | | CCATCTACAGACCTGCAGCTTCATAAAACTTCTGATTTCTCTTCAG |
| | | CTTTGAAAAGGGTTACCCTGGGCACTGGCCTAGAGCCTCACCTCC |
| | | TAATAGACTTAGCCCCATGAGTTTGCCATGTTGAGCAGGACTATT |
| | | TCTGGCACTTGCAAGTCCCATGATTTCTTCGGTAATTCTGAGGGTG |
| | | GGGGGAGGGACATGAAATCATCTTAGCTTAGCTTTCTGTCTGTGA |
| | | ATGTCTATATAGTGTATTGTGTGTTTTAACAAATGATTTACACTGA |
| | | CTGTTGCTGTAAAAGTGAATTTGGAAATAAAGTTATTACTCTGAT |
| | | TAAA |
| 38 | 2 | GCAGTCACCGCCACCCACCAGCTCCGGCACCAACAGCAGCGCCG |
| | | CTGCCACCGCCCACCTTCTGCCGCCGCCACCACAGCCACCTTCTCC |
| | | TCCTCCGCTGTCCTCTCCCGTCCTCGCCTCTGTCGACTATCAGGTG |
| | | AACTTTGAACCAGGATGGCTGAGCCCCGCCAGGAGTTCGAAGTG |
| | | ATGGAAGATCACGCTGGGACGTACGGGTTGGGGGACAGGAAAGA |
| | | TCAGGGGGGCTACACCATGCACCAAGACCAAGAGGGTGACACGG |
| | | ACGCTGGCCTGAAAGAATCTCCCCTGCAGACCCCCACTGAGGACG |
| | | GATCTGAGGAACCGGGCTCTGAAACCTCTGATGCTAAGAGCACTC |
| | | CAACAGCGGAAGATGTGACAGCACCCTTAGTGGATGAGGGAGCT |
| | | CCCGGCAAGCAGGCTGCCGCGCAGCCCCACACGGAGATCCCAGA |
| | | AGGAACCACAGCTGAAGAAGCAGGCATTGGAGACACCCCCAGCC |
| | | TGGAAGACGAAGCTGCTGGTCACGTGACCCAAGCTCGCATGGTCA |
| | | GTAAAAGCAAAGACGGGACTGGAAGCGATGACAAAAAAGCCAA |
| | | GGGGGCTGATGGTAAAACGAAGATCGCCACACCGCGGGGAGCAG |
| | | CCCCTCCAGGCCAGAAGGGCCAGGCCAACGCCACCAGGATTCCA |
| | | GCAAAAACCCCGCCCGCTCCAAAGACACCACCCAGCTCTGGTGA |
| | | ACCTCCAAAATCAGGGGATCGCAGCGGCTACAGCAGCCCCGGCT |
| | | CCCCAGGCACTCCCGGCAGCCGCTCCCGCACCCCGTCCCTTCCAA |
| | | CCCCACCCACCCGGGAGCCCAAGAAGGTGGCAGTGGTCCGTACTC |
| | | CACCCAAGTCGCCGTCTTCCGCCAAGAGCCGCCTGCAGACAGCCC |
| | | CCGTGCCCATGCCAGACCTGAAGAATGTCAAGTCCAAGATCGGCT |
| | | CCACTGAGAACCTGAAGCACCAGCCGGGAGGCGGGAAGGTGCAG |
| | | ATAATTAATAAGAAGCTGGATCTTAGCAACGTCCAGTCCAAGTGT |
| | | GGCTCAAAGGATAATATCAAACACGTCCCGGGAGGCGGCAGTGT |
| | | GCAAATAGTCTACAAACCAGTTGACCTGAGCAAGGTGACCTCCAA |
| | | GTGTGGCTCATTAGGCAACATCCATCATAAACCAGGAGGTGGCCA |
| | | GGTGGAAGTAAAATCTGAGAAGCTTGACTTCAAGGACAGAGTCC |
| | | AGTCGAAGATTGGGTCCCTGGACAATATCACCCACGTCCCTGGCG |
| | | GAGGAAATAAAAAGATTGAAACCCACAAGCTGACCTTCCGCGAG |
| | | AACGCCAAAGCCAAGACAGACCACGGGGCGGAGATCGTGTACAA |
| | | GTCGCCAGTGGTGTCTGGGGACACGTCTCCACGGCATCTCAGCAA |
| | | TGTCTCCTCCACCGGCAGCATCGACATGGTAGACTCGCCCCAGCT |
| | | CGCCACGCTAGCTGACGAGGTGTCTGCCTCCCTGGCCAAGCAGGG |
| | | TTTGTGATCAGGCCCCTGGGGCGGTCAATAATTGTGGAGAGGAGA |
| | | GAATGAGAGAGTGTGGAAAAAAAAAGAATAATGACCCGGCCCCC |
| | | GCCCTCTGCCCCCAGCTGCTCCTCGCAGTTCGGTTAATTGGTTAAT |
| | | CACTTAACCTGCTTTTGTCACTCGGCTTTGGCTCGGGACTTCAAAA |
| | | TCAGTGATGGGAGTAAGAGCAAATTTCATCTTTCCAAATTGATGG |
| | | GTGGGCTAGTAATAAAATATTTAAAAAAAAACATTCAAAAACAT |
| | | GGCCACATCCAACATTTCCTCAGGCAATTCCTTTTGATTCTTTTTT |
| | | CTTCCCCCTCCATGTAGAAGAGGGAGAAGGAGAGGCTCTGAAAG |
| | | CTGCTTCTGGGGGATTTCAAGGGACTGGGGGTGCCAACCACCTCT |
| | | GGCCCTGTTGTGGGGGTGTCACAGAGGCAGTGGCAGCAACAAAG |
| | | GATTTGAAACTTGGTGTGTTCGTGGAGCCACAGGCAGACGATGTC |
| | | AACCTTGTGTGAGTGTGACGGGGGTTGGGGTGGGGCGGGAGGCC |
| | | ACGGGGGAGGCCGAGGCAGGGGCTGGGCAGAGGGGAGAGGAAG |
| | | CACAAGAAGTGGGAGTGGGAGAGGAAGCCACGTGCTGGAGAGTA |
| | | GACATCCCCCTCCTTGCCGCTGGGAGAGCCAAGGCCTATGCCACC |
| | | TGCAGCGTCTGAGCGGCCGCCTGTCCTTGGTGGCCGGGGGTGGGG |
| | | GCCTGCTGTGGGTCAGTGTGCCACCCTCTGCAGGGCAGCCTGTGG |
| | | GAGAAGGGACAGCGGGTAAAAAGAGAAGGCAAGCTGGCAGGAG |
| | | GGTGGCACTTCGTGGATGACCTCCTTAGAAAAGACTGACCTTGAT |
| | | GTCTTGAGAGCGCTGGCCTCTTCCTCCCTCCCTGCAGGGTAGGGG |
| | | GCCTGAGTTGAGGGGCTTCCCTCTGCTCCACAGAAACCCTGTTTT |
| | | ATTGAGTTCTGAAGGTTGGAACTGCTGCCATGATTTTGGCCACTTT |
| | | GCAGACCTGGGACTTTAGGGCTAACCAGTTCTCTTTGTAAGGACT |
| | | TGTGCCTCTTGGGAGACGTCCACCCGTTTCCAAGCCTGGGCCACT |
| | | GGCATCTCTGGAGTGTGTGGGGGTCTGGGAGGCAGGTCCCGAGCC |
| | | CCCTGTCCTTCCCACGGCCACTGCAGTCACCCCGTCTGCGCCGCTG |
| | | TGCTGTTGTCTGCCGTGAGAGCCCAATCACTGCCTATACCCCTCAT |
| | | CACACGTCACAATGTCCCGAATTCCCAGCCTCACCACCCCTTCTC |
| | | AGTAATGACCCTGGTTGGTTGCAGGAGGTACCTACTCCATACTGA |
| | | GGGTGAAATTAAGGGAAGGCAAAGTCCAGGCACAAGAGTGGGAC |
| | | CCCAGCCTCTCACTCTCAGTTCCACTCATCCAACTGGGACCCTCAC |
| | | CACGAATCTCATGATCTGATTCGGTTCCCTGTCTCCTCCTCCCGTC |
| | | ACAGATGTGAGCCAGGGCACTGCTCAGCTGTGACCCTAGGTGTTT |
| | | CTGCCTTGTTGACATGGAGAGAGCCCTTTCCCCTGAGAAGGCCTG |
| | | GCCCCTTCCTGTGCTGAGCCCACAGCAGCAGGCTGGGTGTCTTGG |
| | | TTGTCAGTGGTGGCACCAGGATGGAAGGGCAAGGCACCCAGGGC |
| | | AGGCCCACAGTCCCGCTGTCCCCCACTTGCACCCTAGCTTGTAGC |
| | | TGCCAACCTCCCAGACAGCCCAGCCCGCTGCTCAGCTCCACATGC |
| | | ATAGTATCAGCCCTCCACACCCGACAAAGGGGAACACACCCCCTT |
| | | GGAAATGGTTCTTTTCCCCCAGTCCCAGCTGGAAGCCATGCTGTC |
| | | TGTTCTGCTGGAGCAGCTGAACATATACATAGATGTTGCCCTGCC |
| | | CTCCCCATCTGCACCCTGTTGAGTTGTAGTTGGATTTGTCTGTTTA |
| | | TGCTTGGATTCACCAGAGTGACTATGATAGTGAAAAGAAAAAAA |
| | | AAAAAAAAAAAGGACGCATGTATCTTGAAATGCTTGTAAAGAGG |
| | | TTTCTAACCCACCCTCACGAGGTGTCTCTCACCCCCACACTGGGA |
| | | CTCGTGTGGCCTGTGTGGTGCCACCCTGCTGGGGCCTCCCAAGTTT |
| | | TGAAAGGCTTTCCTCAGCACCTGGGACCCAACAGAGACCAGCTTC |
| | | TAGCAGCTAAGGAGGCCGTTCAGCTGTGACGAAGGCCTGAAGCA |
| | | CAGGATTAGGACTGAAGCGATGATGTCCCCTTCCCTACTTCCCCTT |
| | | GGGGCTCCCTGTGTCAGGGCACAGACTAGGTCTTGTGGCTGGTCT |
| | | GGCTTGCGGCGCGAGGATGGTTCTCTCTGGTCATAGCCCGAAGTC |
| | | TCATGGCAGTCCCAAAGGAGGCTTACAACTCCTGCATCACAAGAA |
| | | AAAGGAAGCCACTGCCAGCTGGGGGGATCTGCAGCTCCCAGAAG |
| | | CTCCGTGAGCCTCAGCCACCCCTCAGACTGGGTTCCTCTCCAAGC |
| | | TCGCCCTCTGGAGGGGCAGCGCAGCCTCCCACCAAGGGCCCTGCG |
| | | ACCACAGCAGGGATTGGGATGAATTGCCTGTCCTGGATCTGCTCT |
| | | AGAGGCCCAAGCTGCCTGCCTGAGGAAGGATGACTTGACAAGTC |
| | | AGGAGACACTGTTCCCAAAGCCTTGACCAGAGCACCTCAGCCCGC |
| | | TGACCTTGCACAAACTCCATCTGCTGCCATGAGAAAAGGGAAGCC |
| | | GCCTTTGCAAAACATTGCTGCCTAAAGAAACTCAGCAGCCTCAGG |
| | | CCCAATTCTGCCACTTCTGGTTTGGGTACAGTTAAAGGCAACCCT |
| | | GAGGGACTTGGCAGTAGAAATCCAGGGCCTCCCCTGGGGCTGGC |
| | | AGCTTCGTGTGCAGCTAGAGCTTTACCTGAAAGGAAGTCTCTGGG |
| | | CCCAGAACTCTCCACCAAGAGCCTCCCTGCCGTTCGCTGAGTCCC |
| | | AGCAATTCTCCTAAGTTGAAGGGATCTGAGAAGGAGAAGGAAAT |
| | | GTGGGGTAGATTTGGTGGTGGTTAGAGATATGCCCCCCTCATTAC |
| | | TGCCAACAGTTTCGGCTGCATTTCTTCACGCACCTCGGTTCCTCTT |
| | | CCTGAAGTTCTTGTGCCCTGCTCTTCAGCACCATGGGCCTTCTTAT |
| | | ACGGAAGGCTCTGGGATCTCCCCCTTGTGGGGCAGGCTCTTGGGG |
| | | CCAGCCTAAGATCATGGTTTAGGGTGATCAGTGCTGGCAGATAAA |
| | | TTGAAAAGGCACGCTGGCTTGTGATCTTAAATGAGGACAATCCCC |
| | | CCAGGGCTGGGCACTCCTCCCCTCCCCTCACTTCTCCCACCTGCAG |
| | | AGCCAGTGTCCTTGGGTGGGCTAGATAGGATATACTGTATGCCGG |
| | | CTCCTTCAAGCTGCTGACTCACTTTATCAATAGTTCCATTTAAATT |
| | | GACTTCAGTGGTGAGACTGTATCCTGTTTGCTATTGCTTGTTGTGC |
| | | TATGGGGGGAGGGGGGAGGAATGTGTAAGATAGTTAACATGGGC |
| | | AAAGGGAGATCTTGGGGTGCAGCACTTAAACTGCCTCGTAACCCT |
| | | TTTCATGATTTCAACCACATTTGCTAGAGGGAGGGAGCAGCCACG |
| | | GAGTTAGAGGCCCTTGGGGTTTCTCTTTTCCACTGACAGGCTTTCC |
| | | CAGGCAGCTGGCTAGTTCATTCCCTCCCCAGCCAGGTGCAGGCGT |
| | | AGGAATATGGACATCTGGTTGCTTTGGCCTGCTGCCCTCTTTCAGG |
| | | GGTCCTAAGCCCACAATCATGCCTCCCTAAGACCTTGGCATCCTT |
| | | CCCTCTAAGCCGTTGGCACCTCTGTGCCACCTCTCACACTGGCTCC |
| | | AGACACACAGCCTGTGCTTTTGGAGCTGAGATCACTCGCTTCACC |
| | | CTCCTCATCTTTGTTCTCCAAGTAAAGCCACGAGGTCGGGGCGAG |
| | | GGCAGAGGTGATCACCTGCGTGTCCCATCTACAGACCTGCAGCTT |
| | | CATAAAACTTCTGATTTCTCTTCAGCTTTGAAAAGGGTTACCCTGG |
| | | GCACTGGCCTAGAGCCTCACCTCCTAATAGACTTAGCCCCATGAG |
| | | TTTGCCATGTTGAGCAGGACTATTTCTGGCACTTGCAAGTCCCATG |
| | | ATTTCTTCGGTAATTCTGAGGGTGGGGGGAGGGACATGAAATCAT |
| | | CTTAGCTTAGCTTTCTGTCTGTGAATGTCTATATAGTGTATTGTGT |
| | | GTTTTAACAAATGATTTACACTGACTGTTGCTGTAAAAGTGAATTT |
| | | GGAAATAAAGTTATTACTCTGATTAAA |
| 39 | 3 | GCAGTCACCGCCACCCACCAGCTCCGGCACCAACAGCAGCGCCG |
| | | CTGCCACCGCCCACCTTCTGCCGCCGCCACCACAGCCACCTTCTCC |
| | | TCCTCCGCTGTCCTCTCCCGTCCTCGCCTCTGTCGACTATCAGGTG |
| | | AACTTTGAACCAGGATGGCTGAGCCCCGCCAGGAGTTCGAAGTG |
| | | ATGGAAGATCACGCTGGGACGTACGGGTTGGGGGACAGGAAAGA |
| | | TCAGGGGGGCTACACCATGCACCAAGACCAAGAGGGTGACACGG |
| | | ACGCTGGCCTGAAAGCTGAAGAAGCAGGCATTGGAGACACCCCC |
| | | AGCCTGGAAGACGAAGCTGCTGGTCACGTGACCCAAGCTCGCAT |
| | | GGTCAGTAAAAGCAAAGACGGGACTGGAAGCGATGACAAAAAA |
| | | GCCAAGGGGGCTGATGGTAAAACGAAGATCGCCACACCGCGGGG |
| | | AGCAGCCCCTCCAGGCCAGAAGGGCCAGGCCAACGCCACCAGGA |
| | | TTCCAGCAAAAACCCCGCCCGCTCCAAAGACACCACCCAGCTCTG |
| | | GTGAACCTCCAAAATCAGGGGATCGCAGCGGCTACAGCAGCCCC |
| | | GGCTCCCCAGGCACTCCCGGCAGCCGCTCCCGCACCCCGTCCCTT |
| | | CCAACCCCACCCACCCGGGAGCCCAAGAAGGTGGCAGTGGTCCG |
| | | TACTCCACCCAAGTCGCCGTCTTCCGCCAAGAGCCGCCTGCAGAC |
| | | AGCCCCCGTGCCCATGCCAGACCTGAAGAATGTCAAGTCCAAGAT |
| | | CGGCTCCACTGAGAACCTGAAGCACCAGCCGGGAGGCGGGAAGG |
| | | TGCAGATAATTAATAAGAAGCTGGATCTTAGCAACGTCCAGTCCA |
| | | AGTGTGGCTCAAAGGATAATATCAAACACGTCCCGGGAGGCGGC |
| | | AGTGTGCAAATAGTCTACAAACCAGTTGACCTGAGCAAGGTGACC |
| | | TCCAAGTGTGGCTCATTAGGCAACATCCATCATAAACCAGGAGGT |
| | | GGCCAGGTGGAAGTAAAATCTGAGAAGCTTGACTTCAAGGACAG |
| | | AGTCCAGTCGAAGATTGGGTCCCTGGACAATATCACCCACGTCCC |
| | | TGGCGGAGGAAATAAAAAGATTGAAACCCACAAGCTGACCTTCC |
| | | GCGAGAACGCCAAAGCCAAGACAGACCACGGGGCGGAGATCGTG |
| | | TACAAGTCGCCAGTGGTGTCTGGGGACACGTCTCCACGGCATCTC |
| | | AGCAATGTCTCCTCCACCGGCAGCATCGACATGGTAGACTCGCCC |
| | | CAGCTCGCCACGCTAGCTGACGAGGTGTCTGCCTCCCTGGCCAAG |
| | | CAGGGTTTGTGATCAGGCCCCTGGGGCGGTCAATAATTGTGGAGA |
| | | GGAGAGAATGAGAGAGTGTGGAAAAAAAAAGAATAATGACCCG |
| | | GCCCCCGCCCTCTGCCCCCAGCTGCTCCTCGCAGTTCGGTTAATTG |
| | | GTTAATCACTTAACCTGCTTTTGTCACTCGGCTTTGGCTCGGGACT |
| | | TCAAAATCAGTGATGGGAGTAAGAGCAAATTTCATCTTTCCAAAT |
| | | TGATGGGTGGGCTAGTAATAAAATATTTAAAAAAAAACATTCAA |
| | | AAACATGGCCACATCCAACATTTCCTCAGGCAATTCCTTTTGATTC |
| | | TTTTTTCTTCCCCCTCCATGTAGAAGAGGGAGAAGGAGAGGCTCT |
| | | GAAAGCTGCTTCTGGGGGATTTCAAGGGACTGGGGGTGCCAACC |
| | | ACCTCTGGCCCTGTTGTGGGGGTGTCACAGAGGCAGTGGCAGCAA |
| | | CAAAGGATTTGAAACTTGGTGTGTTCGTGGAGCCACAGGCAGACG |
| | | ATGTCAACCTTGTGTGAGTGTGACGGGGGTTGGGGTGGGGCGGG |
| | | AGGCCACGGGGGAGGCCGAGGCAGGGGCTGGGCAGAGGGGAGA |
| | | GGAAGCACAAGAAGTGGGAGTGGGAGAGGAAGCCACGTGCTGG |
| | | AGAGTAGACATCCCCCTCCTTGCCGCTGGGAGAGCCAAGGCCTAT |
| | | GCCACCTGCAGCGTCTGAGCGGCCGCCTGTCCTTGGTGGCCGGGG |
| | | GTGGGGGCCTGCTGTGGGTCAGTGTGCCACCCTCTGCAGGGCAGC |
| | | CTGTGGGAGAAGGGACAGCGGGTAAAAAGAGAAGGCAAGCTGG |
| | | CAGGAGGGTGGCACTTCGTGGATGACCTCCTTAGAAAAGACTGAC |
| | | CTTGATGTCTTGAGAGCGCTGGCCTCTTCCTCCCTCCCTGCAGGGT |
| | | AGGGGGCCTGAGTTGAGGGGCTTCCCTCTGCTCCACAGAAACCCT |
| | | GTTTTATTGAGTTCTGAAGGTTGGAACTGCTGCCATGATTTTGGCC |
| | | ACTTTGCAGACCTGGGACTTTAGGGCTAACCAGTTCTCTTTGTAA |
| | | GGACTTGTGCCTCTTGGGAGACGTCCACCCGTTTCCAAGCCTGGG |
| | | CCACTGGCATCTCTGGAGTGTGTGGGGGTCTGGGAGGCAGGTCCC |
| | | GAGCCCCCTGTCCTTCCCACGGCCACTGCAGTCACCCCGTCTGCG |
| | | CCGCTGTGCTGTTGTCTGCCGTGAGAGCCCAATCACTGCCTATAC |
| | | CCCTCATCACACGTCACAATGTCCCGAATTCCCAGCCTCACCACC |
| | | CCTTCTCAGTAATGACCCTGGTTGGTTGCAGGAGGTACCTACTCC |
| | | AACTGAGGGGAAATTAAGGGAAGGCAAAGCCAGGCACAAGA |
| | | GTGGGACCCCAGCCTCTCACTCTCAGTTCCACTCATCCAACTGGG |
| | | ACCCTCACCACGAATCTCATGATCTGATTCGGTTCCCTGTCTCCTC |
| | | CTCCCGTCACAGATGTGAGCCAGGGCACTGCTCAGCTGTGACCCT |
| | | AGGTGTTTCTGCCTTGTTGACATGGAGAGAGCCCTTTCCCCTGAG |
| | | AAGGCCTGGCCCCTTCCTGTGCTGAGCCCACAGCAGCAGGCTGGG |
| | | TGTCTTGGTTGTCAGTGGTGGCACCAGGATGGAAGGGCAAGGCAC |
| | | CCAGGGCAGGCCCACAGTCCCGCTGTCCCCCACTTGCACCCTAGC |
| | | TTGTAGCTGCCAACCTCCCAGACAGCCCAGCCCGCTGCTCAGCTC |
| | | CACATGCATAGTATCAGCCCTCCACACCCGACAAAGGGGAACAC |
| | | ACCCCCTTGGAAATGGTTCTTTTCCCCCAGTCCCAGCTGGAAGCC |
| | | ATGCTGTCTGTTCTGCTGGAGCAGCTGAACATATACATAGATGTT |
| | | GCCCTGCCCTCCCCATCTGCACCCTGTTGAGTTGTAGTTGGATTTG |
| | | TCTGTTTATGCTTGGATTCACCAGAGTGACTATGATAGTGAAAAG |
| | | AAAAAAAAAAAAAAAAAAGGACGCATGTATCTTGAAATGCTTGT |
| | | AAAGAGGTTTCTAACCCACCCTCACGAGGTGTCTCTCACCCCCAC |
| | | ACTGGGACTCGTGTGGCCTGTGTGGTGCCACCCTGCTGGGGCCTC |
| | | CCAAGTTTTGAAAGGCTTTCCTCAGCACCTGGGACCCAACAGAGA |
| | | CCAGCTTCTAGCAGCTAAGGAGGCCGTTCAGCTGTGACGAAGGCC |
| | | TGAAGCACAGGATTAGGACTGAAGCGATGATGTCCCCTTCCCTAC |
| | | TTCCCCTTGGGGCTCCCTGTGTCAGGGCACAGACTAGGTCTTGTG |
| | | GCTGGTCTGGCTTGCGGCGCGAGGATGGTTCTCTCTGGTCATAGC |
| | | CCGAAGTCTCATGGCAGTCCCAAAGGAGGCTTACAACTCCTGCAT |
| | | CACAAGAAAAAGGAAGCCACTGCCAGCTGGGGGGATCTGCAGCT |
| | | CCCAGAAGCTCCGTGAGCCTCAGCCACCCCTCAGACTGGGTTCCT |
| | | CTCCAAGCTCGCCCTCTGGAGGGGCAGCGCAGCCTCCCACCAAGG |
| | | GCCCTGCGACCACAGCAGGGATTGGGATGAATTGCCTGTCCTGGA |
| | | TCTGCTCTAGAGGCCCAAGCTGCCTGCCTGAGGAAGGATGACTTG |
| | | ACAAGTCAGGAGACACTGTTCCCAAAGCCTTGACCAGAGCACCTC |
| | | AGCCCGCTGACCTTGCACAAACTCCATCTGCTGCCATGAGAAAAG |
| | | GGAAGCCGCCTTTGCAAAACATTGCTGCCTAAAGAAACTCAGCAG |
| | | CCTCAGGCCCAATTCTGCCACTTCTGGTTTGGGTACAGTTAAAGG |
| | | CAACCCTGAGGGACTTGGCAGTAGAAATCCAGGGCCTCCCCTGGG |
| | | GCTGGCAGCTTCGTGTGCAGCTAGAGCTTTACCTGAAAGGAAGTC |
| | | TCTGGGCCCAGAACTCTCCACCAAGAGCCTCCCTGCCGTTCGCTG |
| | | AGTCCCAGCAATTCTCCTAAGTTGAAGGGATCTGAGAAGGAGAA |
| | | GGAAATGTGGGGTAGATTTGGTGGTGGTTAGAGATATGCCCCCCT |
| | | CATTACTGCCAACAGTTTCGGCTGCATTTCTTCACGCACCTCGGTT |
| | | CCTCTTCCTGAAGTTCTTGTGCCCTGCTCTTCAGCACCATGGGCCT |
| | | TCTTATACGGAAGGCTCTGGGATCTCCCCCTTGTGGGGCAGGCTC |
| | | TTGGGGCCAGCCTAAGATCATGGTTTAGGGTGATCAGTGCTGGCA |
| | | GATAAATTGAAAAGGCACGCTGGCTTGTGATCTTAAATGAGGACA |
| | | ATCCCCCCAGGGCTGGGCACTCCTCCCCTCCCCTCACTTCTCCCAC |
| | | CTGCAGAGCCAGTGTCCTTGGGTGGGCTAGATAGGATATACTGTA |
| | | TGCCGGCTCCTTCAAGCTGCTGACTCACTTTATCAATAGTTCCATT |
| | | TAAATTGACTTCAGTGGTGAGACTGTATCCTGTTTGCTATTGCTTG |
| | | TTGTGCTATGGGGGGAGGGGGGAGGAATGTGTAAGATAGTTAAC |
| | | ATGGGCAAAGGGAGATCTTGGGGTGCAGCACTTAAACTGCCTCGT |
| | | AACCCTTTTCATGATTTCAACCACATTTGCTAGAGGGAGGGAGCA |
| | | GCCACGGAGTTAGAGGCCCTTGGGGTTTCTCTTTTCCACTGACAG |
| | | GCTTTCCCAGGCAGCTGGCTAGTTCATTCCCTCCCCAGCCAGGTG |
| | | CAGGCGTAGGAATATGGACATCTGGTTGCTTTGGCCTGCTGCCCT |
| | | CTTTCAGGGGTCCTAAGCCCACAATCATGCCTCCCTAAGACCTTG |
| | | GCATCCTTCCCTCTAAGCCGTTGGCACCTCTGTGCCACCTCTCACA |
| | | CTGGCTCCAGACACACAGCCTGTGCTTTTGGAGCTGAGATCACTC |
| | | GCTTCACCCTCCTCATCTTTGTTCTCCAAGTAAAGCCACGAGGTCG |
| | | GGGCGAGGGCAGAGGTGATCACCTGCGTGTCCCATCTACAGACCT |
| | | GCAGCTTCATAAAACTTCTGATTTCTCTTCAGCTTTGAAAAGGGTT |
| | | ACCCTGGGCACTGGCCTAGAGCCTCACCTCCTAATAGACTTAGCC |
| | | CCATGAGTTTGCCATGTTGAGCAGGACTATTTCTGGCACTTGCAA |
| | | GTCCCATGATTTCTTCGGTAATTCTGAGGGTGGGGGGAGGGACAT |
| | | GAAATCATCTTAGCTTAGCTTTCTGTCTGTGAATGTCTATATAGTG |
| | | TATTGTGTGTTTTAACAAATGATTTACACTGACTGTTGCTGTAAAA |
| | | GTGAATTTGGAAATAAAGTTATTACTCTGATTAAA |
| 40 | 4 | GCAGTCACCGCCACCCACCAGCTCCGGCACCAACAGCAGCGCCG |
| | | CTGCCACCGCCCACCTTCTGCCGCCGCCACCACAGCCACCTTCTCC |
| | | TCCTCCGCTGTCCTCTCCCGTCCTCGCCTCTGTCGACTATCAGGTG |
| | | AACTTTGAACCAGGATGGCTGAGCCCCGCCAGGAGTTCGAAGTG |
| | | ATGGAAGATCACGCTGGGACGTACGGGTTGGGGGACAGGAAAGA |
| | | TCAGGGGGGCTACACCATGCACCAAGACCAAGAGGGTGACACGG |
| | | ACGCTGGCCTGAAAGCTGAAGAAGCAGGCATTGGAGACACCCCC |
| | | AGCCTGGAAGACGAAGCTGCTGGTCACGTGACCCAAGCTCGCAT |
| | | GGTCAGTAAAAGCAAAGACGGGACTGGAAGCGATGACAAAAAA |
| | | GCCAAGGGGGCTGATGGTAAAACGAAGATCGCCACACCGCGGGG |
| | | AGCAGCCCCTCCAGGCCAGAAGGGCCAGGCCAACGCCACCAGGA |
| | | TTCCAGCAAAAACCCCGCCCGCTCCAAAGACACCACCCAGCTCTG |
| | | GTGAACCTCCAAAATCAGGGGATCGCAGCGGCTACAGCAGCCCC |
| | | GGCTCCCCAGGCACTCCCGGCAGCCGCTCCCGCACCCCGTCCCTT |
| | | CCAACCCCACCCACCCGGGAGCCCAAGAAGGTGGCAGTGGTCCG |
| | | TACTCCACCCAAGTCGCCGTCTTCCGCCAAGAGCCGCCTGCAGAC |
| | | AGCCCCCGTGCCCATGCCAGACCTGAAGAATGTCAAGTCCAAGAT |
| | | CGGCTCCACTGAGAACCTGAAGCACCAGCCGGGAGGCGGGAAGG |
| | | TGCAAATAGTCTACAAACCAGTTGACCTGAGCAAGGTGACCTCCA |
| | | AGTGTGGCTCATTAGGCAACATCCATCATAAACCAGGAGGTGGCC |
| | | AGGTGGAAGTAAAATCTGAGAAGCTTGACTTCAAGGACAGAGTC |
| | | CAGTCGAAGATTGGGTCCCTGGACAATATCACCCACGTCCCTGGC |
| | | GGAGGAAATAAAAAGATTGAAACCCACAAGCTGACCTTCCGCGA |
| | | GAACGCCAAAGCCAAGACAGACCACGGGGCGGAGATCGTGTACA |
| | | AGTCGCCAGTGGTGTCTGGGGACACGTCTCCACGGCATCTCAGCA |
| | | ATGTCTCCTCCACCGGCAGCATCGACATGGTAGACTCGCCCCAGC |
| | | TCGCCACGCTAGCTGACGAGGTGTCTGCCTCCCTGGCCAAGCAGG |
| | | GTTTGTGATCAGGCCCCTGGGGCGGTCAATAATTGTGGAGAGGAG |
| | | AGAATGAGAGAGTGTGGAAAAAAAAAGAATAATGACCCGGCCCC |
| | | CGCCCTCTGCCCCCAGCTGCTCCTCGCAGTTCGGTTAATTGGTTAA |
| | | TCACTTAACCTGCTTTTGTCACTCGGCTTTGGCTCGGGACTTCAAA |
| | | ATCAGTGATGGGAGTAAGAGCAAATTTCATCTTTCCAAATTGATG |
| | | GGTGGGCTAGTAATAAAATATTTAAAAAAAAACATTCAAAAACA |
| | | TGGCCACATCCAACATTTCCTCAGGCAATTCCTTTTGATTCTTTTT |
| | | TCTTCCCCCTCCATGTAGAAGAGGGAGAAGGAGAGGCTCTGAAA |
| | | GCTGCTTCTGGGGGATTTCAAGGGACTGGGGGTGCCAACCACCTC |
| | | TGGCCCTGTTGTGGGGGTGTCACAGAGGCAGTGGCAGCAACAAA |
| | | GGATTTGAAACTTGGTGTGTTCGTGGAGCCACAGGCAGACGATGT |
| | | CAACCTTGTGTGAGTGTGACGGGGGTTGGGGTGGGGCGGGAGGC |
| | | CACGGGGGAGGCCGAGGCAGGGGCTGGGCAGAGGGGAGAGGAA |
| | | GCACAAGAAGTGGGAGTGGGAGAGGAAGCCACGTGCTGGAGAGT |
| | | AGACATCCCCCTCCTTGCCGCTGGGAGAGCCAAGGCCTATGCCAC |
| | | CTGCAGCGTCTGAGCGGCCGCCTGTCCTTGGTGGCCGGGGGTGGG |
| | | GGCCTGCTGTGGGTCAGTGTGCCACCCTCTGCAGGGCAGCCTGTG |
| | | GGAGAAGGGACAGCGGGTAAAAAGAGAAGGCAAGCTGGCAGGA |
| | | GGGTGGCACTTCGTGGATGACCTCCTTAGAAAAGACTGACCTTGA |
| | | TGTCTTGAGAGCGCTGGCCTCTTCCTCCCTCCCTGCAGGGTAGGG |
| | | GGCCTGAGTTGAGGGGCTTCCCTCTGCTCCACAGAAACCCTGTTT |
| | | TATTGAGTTCTGAAGGTTGGAACTGCTGCCATGATTTTGGCCACTT |
| | | TGCAGACCTGGGACTTTAGGGCTAACCAGTTCTCTTTGTAAGGAC |
| | | TTGTGCCTCTTGGGAGACGTCCACCCGTTTCCAAGCCTGGGCCAC |
| | | TGGCATCTCTGGAGTGTGTGGGGGTCTGGGAGGCAGGTCCCGAGC |
| | | CCCCTGTCCTTCCCACGGCCACTGCAGTCACCCCGTCTGCGCCGCT |
| | | GTGCTGTTGTCTGCCGTGAGAGCCCAATCACTGCCTATACCCCTC |
| | | ATCACACGTCACAATGTCCCGAATTCCCAGCCTCACCACCCCTTCT |
| | | CAGTAATGACCCTGGTTGGTTGCAGGAGGTACCTACTCCATACTG |
| | | AGGGTGAAATTAAGGGAAGGCAAAGTCCAGGCACAAGAGTGGGA |
| | | CCCCAGCCTCTCACTCTCAGTTCCACTCATCCAACTGGGACCCTCA |
| | | CCACGAATCTCATGATCTGATTCGGTTCCCTGTCTCCTCCTCCCGT |
| | | CACAGATGTGAGCCAGGGCACTGCTCAGCTGTGACCCTAGGTGTT |
| | | TCTGCCTTGTTGACATGGAGAGAGCCCTTTCCCCTGAGAAGGCCT |
| | | GGCCCCTTCCTGTGCTGAGCCCACAGCAGCAGGCTGGGTGTCTTG |
| | | GTTGTCAGTGGTGGCACCAGGATGGAAGGGCAAGGCACCCAGGG |
| | | CAGGCCCACAGTCCCGCTGTCCCCCACTTGCACCCTAGCTTGTAG |
| | | CTGCCAACCTCCCAGACAGCCCAGCCCGCTGCTCAGCTCCACATG |
| | | CATAGTATCAGCCCTCCACACCCGACAAAGGGGAACACACCCCCT |
| | | TGGAAATGGTTCTTTTCCCCCAGTCCCAGCTGGAAGCCATGCTGT |
| | | CTGTTCTGCTGGAGCAGCTGAACATATACATAGATGTTGCCCTGC |
| | | CCTCCCCATCTGCACCCTGTTGAGTTGTAGTTGGATTTGTCTGTTT |
| | | ATGCTTGGATTCACCAGAGTGACTATGATAGTGAAAAGAAAAAA |
| | | AAAAAAAAAAAAGGACGCATGTATCTTGAAATGCTTGTAAAGAG |
| | | GTTTCTAACCCACCCTCACGAGGTGTCTCTCACCCCCACACTGGG |
| | | ACTCGTGTGGCCTGTGTGGTGCCACCCTGCTGGGGCCTCCCAAGT |
| | | TTTGAAAGGCTTTCCTCAGCACCTGGGACCCAACAGAGACCAGCT |
| | | TCTAGCAGCTAAGGAGGCCGTTCAGCTGTGACGAAGGCCTGAAG |
| | | CACAGGATTAGGACTGAAGCGATGATGTCCCCTTCCCTACTTCCC |
| | | CTTGGGGCTCCCTGTGTCAGGGCACAGACTAGGTCTTGTGGCTGG |
| | | TCTGGCTTGCGGCGCGAGGATGGTTCTCTCTGGTCATAGCCCGAA |
| | | GTCTCATGGCAGTCCCAAAGGAGGCTTACAACTCCTGCATCACAA |
| | | GAAAAAGGAAGCCACTGCCAGCTGGGGGGATCTGCAGCTCCCAG |
| | | AAGCTCCGTGAGCCTCAGCCACCCCTCAGACTGGGTTCCTCTCCA |
| | | AGCTCGCCCTCTGGAGGGGCAGCGCAGCCTCCCACCAAGGGCCCT |
| | | GCGACCACAGCAGGGATTGGGATGAATTGCCTGTCCTGGATCTGC |
| | | TCTAGAGGCCCAAGCTGCCTGCCTGAGGAAGGATGACTTGACAA |
| | | GTCAGGAGACACTGTTCCCAAAGCCTTGACCAGAGCACCTCAGCC |
| | | CGCTGACCTTGCACAAACTCCATCTGCTGCCATGAGAAAAGGGAA |
| | | GCCGCCTTTGCAAAACATTGCTGCCTAAAGAAACTCAGCAGCCTC |
| | | AGGCCCAATTCTGCCACTTCTGGTTTGGGTACAGTTAAAGGCAAC |
| | | CCTGAGGGACTTGGCAGTAGAAATCCAGGGCCTCCCCTGGGGCTG |
| | | GCAGCTTCGTGTGCAGCTAGAGCTTTACCTGAAAGGAAGTCTCTG |
| | | GGCCCAGAACTCTCCACCAAGAGCCTCCCTGCCGTTCGCTGAGTC |
| | | CCAGCAATTCTCCTAAGTTGAAGGGATCTGAGAAGGAGAAGGAA |
| | | ATGTGGGGTAGATTTGGTGGTGGTTAGAGATATGCCCCCCTCATT |
| | | ACTGCCAACAGTTTCGGCTGCATTTCTTCACGCACCTCGGTTCCTC |
| | | TTCCTGAAGTTCTTGTGCCCTGCTCTTCAGCACCATGGGCCTTCTT |
| | | ATACGGAAGGCTCTGGGATCTCCCCCTTGTGGGGCAGGCTCTTGG |
| | | GGCCAGCCTAAGATCATGGTTTAGGGTGATCAGTGCTGGCAGATA |
| | | AATTGAAAAGGCACGCTGGCTTGTGATCTTAAATGAGGACAATCC |
| | | CCCCAGGGCTGGGCACTCCTCCCCTCCCCTCACTTCTCCCACCTGC |
| | | AGAGCCAGTGTCCTTGGGTGGGCTAGATAGGATATACTGTATGCC |
| | | GGCTCCTTCAAGCTGCTGACTCACTTTATCAATAGTTCCATTTAAA |
| | | TTGACTTCAGTGGTGAGACTGTATCCTGTTTGCTATTGCTTGTTGT |
| | | GCTATGGGGGGAGGGGGGAGGAATGTGTAAGATAGTTAACATGG |
| | | GCAAAGGGAGATCTTGGGGTGCAGCACTTAAACTGCCTCGTAACC |
| | | CTTTTCATGATTTCAACCACATTTGCTAGAGGGAGGGAGCAGCCA |
| | | CGGAGTTAGAGGCCCTTGGGGTTTCTCTTTTCCACTGACAGGCTTT |
| | | CCCAGGCAGCTGGCTAGTTCATTCCCTCCCCAGCCAGGTGCAGGC |
| | | GTAGGAATATGGACATCTGGTTGCTTTGGCCTGCTGCCCTCTTTCA |
| | | GGGGTCCTAAGCCCACAATCATGCCTCCCTAAGACCTTGGCATCC |
| | | TTCCCTCTAAGCCGTTGGCACCTCTGTGCCACCTCTCACACTGGCT |
| | | CCAGACACACAGCCTGTGCTTTTGGAGCTGAGATCACTCGCTTCA |
| | | CCCTCCTCATCTTTGTTCTCCAAGTAAAGCCACGAGGTCGGGGCG |
| | | AGGGCAGAGGTGATCACCTGCGTGTCCCATCTACAGACCTGCAGC |
| | | TTCATAAAACTTCTGATTTCTCTTCAGCTTTGAAAAGGGTTACCCT |
| | | GGGCACTGGCCTAGAGCCTCACCTCCTAATAGACTTAGCCCCATG |
| | | AGTTTGCCATGTTGAGCAGGACTATTTCTGGCACTTGCAAGTCCC |
| | | ATGATTTCTTCGGTAATTCTGAGGGTGGGGGGAGGGACATGAAAT |
| | | CATCTTAGCTTAGCTTTCTGTCTGTGAATGTCTATATAGTGTATTG |
| | | TGTGTTTTAACAAATGATTTACACTGACTGTTGCTGTAAAAGTGA |
| | | ATTTGGAAATAAAGTTATTACTCTGATTAAA |
| 41 | 5 | GCAGTCACCGCCACCCACCAGCTCCGGCACCAACAGCAGCGCCG |
| | | CTGCCACCGCCCACCTTCTGCCGCCGCCACCACAGCCACCTTCTCC |
| | | TCCTCCGCTGTCCTCTCCCGTCCTCGCCTCTGTCGACTATCAGGTG |
| | | AACTTTGAACCAGGATGGCTGAGCCCCGCCAGGAGTTCGAAGTG |
| | | ATGGAAGATCACGCTGGGACGTACGGGTTGGGGGACAGGAAAGA |
| | | TCAGGGGGGCTACACCATGCACCAAGACCAAGAGGGTGACACGG |
| | | ACGCTGGCCTGAAAGAATCTCCCCTGCAGACCCCCACTGAGGACG |
| | | GATCTGAGGAACCGGGCTCTGAAACCTCTGATGCTAAGAGCACTC |
| | | CAACAGCGGAAGCTGAAGAAGCAGGCATTGGAGACACCCCCAGC |
| | | CTGGAAGACGAAGCTGCTGGTCACGTGACCCAAGCTCGCATGGTC |
| | | AGTAAAAGCAAAGACGGGACTGGAAGCGATGACAAAAAAGCCA |
| | | AGGGGGCTGATGGTAAAACGAAGATCGCCACACCGCGGGGAGCA |
| | | GCCCCTCCAGGCCAGAAGGGCCAGGCCAACGCCACCAGGATTCC |
| | | AGCAAAAACCCCGCCCGCTCCAAAGACACCACCCAGCTCTGGTG |
| | | AACCTCCAAAATCAGGGGATCGCAGCGGCTACAGCAGCCCCGGC |
| | | TCCCCAGGCACTCCCGGCAGCCGCTCCCGCACCCCGTCCCTTCCA |
| | | ACCCCACCCACCCGGGAGCCCAAGAAGGTGGCAGTGGTCCGTAC |
| | | TCCACCCAAGTCGCCGTCTTCCGCCAAGAGCCGCCTGCAGACAGC |
| | | CCCCGTGCCCATGCCAGACCTGAAGAATGTCAAGTCCAAGATCGG |
| | | CTCCACTGAGAACCTGAAGCACCAGCCGGGAGGCGGGAAGGTGC |
| | | AGATAATTAATAAGAAGCTGGATCTTAGCAACGTCCAGTCCAAGT |
| | | GTGGCTCAAAGGATAATATCAAACACGTCCCGGGAGGCGGCAGT |
| | | GTGCAAATAGTCTACAAACCAGTTGACCTGAGCAAGGTGACCTCC |
| | | AAGTGTGGCTCATTAGGCAACATCCATCATAAACCAGGAGGTGGC |
| | | CAGGTGGAAGTAAAATCTGAGAAGCTTGACTTCAAGGACAGAGT |
| | | CCAGTCGAAGATTGGGTCCCTGGACAATATCACCCACGTCCCTGG |
| | | CGGAGGAAATAAAAAGATTGAAACCCACAAGCTGACCTTCCGCG |
| | | AGAACGCCAAAGCCAAGACAGACCACGGGGCGGAGATCGTGTAC |
| | | AAGTCGCCAGTGGTGTCTGGGGACACGTCTCCACGGCATCTCAGC |
| | | AATGTCTCCTCCACCGGCAGCATCGACATGGTAGACTCGCCCCAG |
| | | CTCGCCACGCTAGCTGACGAGGTGTCTGCCTCCCTGGCCAAGCAG |
| | | GGTTTGTGATCAGGCCCCTGGGGCGGTCAATAATTGTGGAGAGGA |
| | | GAGAATGAGAGAGTGTGGAAAAAAAAAGAATAATGACCCGGCCC |
| | | CCGCCCTCTGCCCCCAGCTGCTCCTCGCAGTTCGGTTAATTGGTTA |
| | | ATCACTTAACCTGCTTTTGTCACTCGGCTTTGGCTCGGGACTTCAA |
| | | AATCAGTGATGGGAGTAAGAGCAAATTTCATCTTTCCAAATTGAT |
| | | GGGTGGGCTAGTAATAAAATATTTAAAAAAAAACATTCAAAAAC |
| | | ATGGCCACATCCAACATTTCCTCAGGCAATTCCTTTTGATTCTTTT |
| | | TTCTTCCCCCTCCATGTAGAAGAGGGAGAAGGAGAGGCTCTGAAA |
| | | GCTGCTTCTGGGGGATTTCAAGGGACTGGGGGTGCCAACCACCTC |
| | | TGGCCCTGTTGTGGGGGTGTCACAGAGGCAGTGGCAGCAACAAA |
| | | GGATTTGAAACTTGGTGTGTTCGTGGAGCCACAGGCAGACGATGT |
| | | CAACCTTGTGTGAGTGTGACGGGGGTTGGGGTGGGGCGGGAGGC |
| | | CACGGGGGAGGCCGAGGCAGGGGCTGGGCAGAGGGGAGAGGAA |
| | | GCACAAGAAGTGGGAGTGGGAGAGGAAGCCACGTGCTGGAGAGT |
| | | AGACATCCCCCTCCTTGCCGCTGGGAGAGCCAAGGCCTATGCCAC |
| | | CTGCAGCGTCTGAGCGGCCGCCTGTCCTTGGTGGCCGGGGGTGGG |
| | | GGCCTGCTGTGGGTCAGTGTGCCACCCTCTGCAGGGCAGCCTGTG |
| | | GGAGAAGGGACAGCGGGTAAAAAGAGAAGGCAAGCTGGCAGGA |
| | | GGGTGGCACTTCGTGGATGACCTCCTTAGAAAAGACTGACCTTGA |
| | | TGTCTTGAGAGCGCTGGCCTCTTCCTCCCTCCCTGCAGGGTAGGG |
| | | GGCCTGAGTTGAGGGGCTTCCCTCTGCTCCACAGAAACCCTGTTT |
| | | TATTGAGTTCTGAAGGTTGGAACTGCTGCCATGATTTTGGCCACTT |
| | | TGCAGACCTGGGACTTTAGGGCTAACCAGTTCTCTTTGTAAGGAC |
| | | TTGTGCCTCTTGGGAGACGTCCACCCGTTTCCAAGCCTGGGCCAC |
| | | TGGCATCTCTGGAGTGTGTGGGGGTCTGGGAGGCAGGTCCCGAGC |
| | | CCCCTGTCCTTCCCACGGCCACTGCAGTCACCCCGTCTGCGCCGCT |
| | | GTGCTGTTGTCTGCCGTGAGAGCCCAATCACTGCCTATACCCCTC |
| | | ATCACACGTCACAATGTCCCGAATTCCCAGCCTCACCACCCCTTCT |
| | | CAGTAATGACCCTGGTTGGTTGCAGGAGGTACCTACTCCATACTG |
| | | AGGGTGAAATTAAGGGAAGGCAAAGTCCAGGCACAAGAGTGGGA |
| | | CCCCAGCCTCTCACTCTCAGTTCCACTCATCCAACTGGGACCCTCA |
| | | CCACGAATCTCATGATCTGATTCGGTTCCCTGTCTCCTCCTCCCGT |
| | | CACAGATGTGAGCCAGGGCACTGCTCAGCTGTGACCCTAGGTGTT |
| | | TCTGCCTTGTTGACATGGAGAGAGCCCTTTCCCCTGAGAAGGCCT |
| | | GGCCCCTTCCTGTGCTGAGCCCACAGCAGCAGGCTGGGTGTCTTG |
| | | GTTGTCAGTGGTGGCACCAGGATGGAAGGGCAAGGCACCCAGGG |
| | | CAGGCCCACAGTCCCGCTGTCCCCCACTTGCACCCTAGCTTGTAG |
| | | CTGCCAACCTCCCAGACAGCCCAGCCCGCTGCTCAGCTCCACATG |
| | | CATAGTATCAGCCCTCCACACCCGACAAAGGGGAACACACCCCCT |
| | | TGGAAATGGTTCTTTTCCCCCAGTCCCAGCTGGAAGCCATGCTGT |
| | | CTGTTCTGCTGGAGCAGCTGAACATATACATAGATGTTGCCCTGC |
| | | CCTCCCCATCTGCACCCTGTTGAGTTGTAGTTGGATTTGTCTGTTT |
| | | ATGCTTGGATTCACCAGAGTGACTATGATAGTGAAAAGAAAAAA |
| | | AAAAAAAAAAAAGGACGCATGTATCTTGAAATGCTTGTAAAGAG |
| | | GTTTCTAACCCACCCTCACGAGGTGTCTCTCACCCCCACACTGGG |
| | | ACTCGTGTGGCCTGTGTGGTGCCACCCTGCTGGGGCCTCCCAAGT |
| | | TTTGAAAGGCTTTCCTCAGCACCTGGGACCCAACAGAGACCAGCT |
| | | TCTAGCAGCTAAGGAGGCCGTTCAGCTGTGACGAAGGCCTGAAG |
| | | CACAGGATTAGGACTGAAGCGATGATGTCCCCTTCCCTACTTCCC |
| | | CTTGGGGCTCCCTGTGTCAGGGCACAGACTAGGTCTTGTGGCTGG |
| | | TCTGGCTTGCGGCGCGAGGATGGTTCTCTCTGGTCATAGCCCGAA |
| | | GTCTCATGGCAGTCCCAAAGGAGGCTTACAACTCCTGCATCACAA |
| | | GAAAAAGGAAGCCACTGCCAGCTGGGGGGATCTGCAGCTCCCAG |
| | | AAGCTCCGTGAGCCTCAGCCACCCCTCAGACTGGGTTCCTCTCCA |
| | | AGCTCGCCCTCTGGAGGGGCAGCGCAGCCTCCCACCAAGGGCCCT |
| | | GCGACCACAGCAGGGATTGGGATGAATTGCCTGTCCTGGATCTGC |
| | | TCTAGAGGCCCAAGCTGCCTGCCTGAGGAAGGATGACTTGACAA |
| | | GTCAGGAGACACTGTTCCCAAAGCCTTGACCAGAGCACCTCAGCC |
| | | CGCTGACCTTGCACAAACTCCATCTGCTGCCATGAGAAAAGGGAA |
| | | GCCGCCTTTGCAAAACATTGCTGCCTAAAGAAACTCAGCAGCCTC |
| | | AGGCCCAATTCTGCCACTTCTGGTTTGGGTACAGTTAAAGGCAAC |
| | | CCTGAGGGACTTGGCAGTAGAAATCCAGGGCCTCCCCTGGGGCTG |
| | | GCAGCTTCGTGTGCAGCTAGAGCTTTACCTGAAAGGAAGTCTCTG |
| | | GGCCCAGAACTCTCCACCAAGAGCCTCCCTGCCGTTCGCTGAGTC |
| | | CCAGCAATTCTCCTAAGTTGAAGGGATCTGAGAAGGAGAAGGAA |
| | | ATGTGGGGTAGATTTGGTGGTGGTTAGAGATATGCCCCCCTCATT |
| | | ACTGCCAACAGTTTCGGCTGCATTTCTTCACGCACCTCGGTTCCTC |
| | | TTCCTGAAGTTCTTGTGCCCTGCTCTTCAGCACCATGGGCCTTCTT |
| | | ATACGGAAGGCTCTGGGATCTCCCCCTTGTGGGGCAGGCTCTTGG |
| | | GGCCAGCCTAAGATCATGGTTTAGGGTGATCAGTGCTGGCAGATA |
| | | AATTGAAAAGGCACGCTGGCTTGTGATCTTAAATGAGGACAATCC |
| | | CCCCAGGGCTGGGCACTCCTCCCCTCCCCTCACTTCTCCCACCTGC |
| | | AGAGCCAGTGTCCTTGGGTGGGCTAGATAGGATATACTGTATGCC |
| | | GGCTCCTTCAAGCTGCTGACTCACTTTATCAATAGTTCCATTTAAA |
| | | TTGACTTCAGTGGTGAGACTGTATCCTGTTTGCTATTGCTTGTTGT |
| | | GCTATGGGGGGAGGGGGGAGGAATGTGTAAGATAGTTAACATGG |
| | | GCAAAGGGAGATCTTGGGGTGCAGCACTTAAACTGCCTCGTAACC |
| | | CTTTTCATGATTTCAACCACATTTGCTAGAGGGAGGGAGCAGCCA |
| | | CGGAGTTAGAGGCCCTTGGGGTTTCTCTTTTCCACTGACAGGCTTT |
| | | CCCAGGCAGCTGGCTAGTTCATTCCCTCCCCAGCCAGGTGCAGGC |
| | | GTAGGAATATGGACATCTGGTTGCTTTGGCCTGCTGCCCTCTTTCA |
| | | GGGGTCCTAAGCCCACAATCATGCCTCCCTAAGACCTTGGCATCC |
| | | TTCCCTCTAAGCCGTTGGCACCTCTGTGCCACCTCTCACACTGGCT |
| | | CCAGACACACAGCCTGTGCTTTTGGAGCTGAGATCACTCGCTTCA |
| | | CCCTCCTCATCTTTGTTCTCCAAGTAAAGCCACGAGGTCGGGGCG |
| | | AGGGCAGAGGTGATCACCTGCGTGTCCCATCTACAGACCTGCAGC |
| | | TTCATAAAACTTCTGATTTCTCTTCAGCTTTGAAAAGGGTTACCCT |
| | | GGGCACTGGCCTAGAGCCTCACCTCCTAATAGACTTAGCCCCATG |
| | | AGTTTGCCATGTTGAGCAGGACTATTTCTGGCACTTGCAAGTCCC |
| | | ATGATTTCTTCGGTAATTCTGAGGGTGGGGGGAGGGACATGAAAT |
| | | CATCTTAGCTTAGCTTTCTGTCTGTGAATGTCTATATAGTGTATTG |
| | | TGTGTTTTAACAAATGATTTACACTGACTGTTGCTGTAAAAGTGA |
| | | ATTTGGAAATAAAGTTATTACTCTGATTAAA |
| 42 | 6 | GCAGTCACCGCCACCCACCAGCTCCGGCACCAACAGCAGCGCCG |
| | | CTGCCACCGCCCACCTTCTGCCGCCGCCACCACAGCCACCTTCTCC |
| | | TCCTCCGCTGTCCTCTCCCGTCCTCGCCTCTGTCGACTATCAGGTG |
| | | AACTTTGAACCAGGATGGCTGAGCCCCGCCAGGAGTTCGAAGTG |
| | | ATGGAAGATCACGCTGGGACGTACGGGTTGGGGGACAGGAAAGA |
| | | TCAGGGGGGCTACACCATGCACCAAGACCAAGAGGGTGACACGG |
| | | ACGCTGGCCTGAAAGAATCTCCCCTGCAGACCCCCACTGAGGACG |
| | | GATCTGAGGAACCGGGCTCTGAAACCTCTGATGCTAAGAGCACTC |
| | | CAACAGCGGAAGATGTGACAGCACCCTTAGTGGATGAGGGAGCT |
| | | CCCGGCAAGCAGGCTGCCGCGCAGCCCCACACGGAGATCCCAGA |
| | | AGGAACCACAGCTGAAGAAGCAGGCATTGGAGACACCCCCAGCC |
| | | TGGAAGACGAAGCTGCTGGTCACGTGACCCAAGAGCCTGAAAGT |
| | | GGTAAGGTGGTCCAGGAAGGCTTCCTCCGAGAGCCAGGCCCCCC |
| | | AGGTCTGAGCCACCAGCTCATGTCCGGCATGCCTGGGGCTCCCCT |
| | | CCTGCCTGAGGGCCCCAGAGAGGCCACACGCCAACCTTCGGGGA |
| | | CAGGACCTGAGGACACAGAGGGCGGCCGCCACGCCCCTGAGCTG |
| | | CTCAAGCACCAGCTTCTAGGAGACCTGCACCAGGAGGGGCCGCC |
| | | GCTGAAGGGGGCAGGGGGCAAAGAGAGGCCGGGGAGCAAGGAG |
| | | GAGGTGGATGAAGACCGCGACGTCGATGAGTCCTCCCCCCAAGA |
| | | CTCCCCTCCCTCCAAGGCCTCCCCAGCCCAAGATGGGCGGCCTCC |
| | | CCAGACAGCCGCCAGAGAAGCCACCAGCATCCCAGGCTTCCCAG |
| | | CGGAGGGTGCCATCCCCCTCCCTGTGGATTTCCTCTCCAAAGTTTC |
| | | CACAGAGATCCCAGCCTCAGAGCCCGACGGGCCCAGTGTAGGGC |
| | | GGGCCAAAGGGCAGGATGCCCCCCTGGAGTTCACGTTTCACGTGG |
| | | AAATCACACCCAACGTGCAGAAGGAGCAGGCGCACTCGGAGGAG |
| | | CATTTGGGAAGGGCTGCATTTCCAGGGGCCCCTGGAGAGGGGCC |
| | | AGAGGCCCGGGGCCCCTCTTTGGGAGAGGACACAAAAGAGGCTG |
| | | ACCTTCCAGAGCCCTCTGAAAAGCAGCCTGCTGCTGCTCCGCGGG |
| | | GGAAGCCCGTCAGCCGGGTCCCTCAACTCAAAGCTCGCATGGTCA |
| | | GTAAAAGCAAAGACGGGACTGGAAGCGATGACAAAAAAGCCAA |
| | | GACATCCACACGTTCCTCTGCTAAAACCTTGAAAAATAGGCCTTG |
| | | CCTTAGCCCCAAACACCCCACTCCTGGTAGCTCAGACCCTCTGAT |
| | | CCAACCCTCCAGCCCTGCTGTGTGCCCAGAGCCACCTTCCTCTCCT |
| | | AAATACGTCTCTTCTGTCACTTCCCGAACTGGCAGTTCTGGAGCA |
| | | AAGGAGATGAAACTCAAGGGGGCTGATGGTAAAACGAAGATCGC |
| | | CACACCGCGGGGAGCAGCCCCTCCAGGCCAGAAGGGCCAGGCCA |
| | | ACGCCACCAGGATTCCAGCAAAAACCCCGCCCGCTCCAAAGACA |
| | | CCACCCAGCTCTGCGACTAAGCAAGTCCAGAGAAGACCACCCCCT |
| | | GCAGGGCCCAGATCTGAGAGAGGTGAACCTCCAAAATCAGGGGA |
| | | TCGCAGCGGCTACAGCAGCCCCGGCTCCCCAGGCACTCCCGGCAG |
| | | CCGCTCCCGCACCCCGTCCCTTCCAACCCCACCCACCCGGGAGCC |
| | | CAAGAAGGTGGCAGTGGTCCGTACTCCACCCAAGTCGCCGTCTTC |
| | | CGCCAAGAGCCGCCTGCAGACAGCCCCCGTGCCCATGCCAGACCT |
| | | GAAGAATGTCAAGTCCAAGATCGGCTCCACTGAGAACCTGAAGC |
| | | ACCAGCCGGGAGGCGGGAAGGTGCAGATAATTAATAAGAAGCTG |
| | | GATCTTAGCAACGTCCAGTCCAAGTGTGGCTCAAAGGATAATATC |
| | | AAACACGTCCCGGGAGGCGGCAGTGTGCAAATAGTCTACAAACC |
| | | AGTTGACCTGAGCAAGGTGACCTCCAAGTGTGGCTCATTAGGCAA |
| | | CATCCATCATAAACCAGGAGGTGGCCAGGTGGAAGTAAAATCTG |
| | | AGAAGCTTGACTTCAAGGACAGAGTCCAGTCGAAGATTGGGTCCC |
| | | TGGACAATATCACCCACGTCCCTGGCGGAGGAAATAAAAAGATT |
| | | GAAACCCACAAGCTGACCTTCCGCGAGAACGCCAAAGCCAAGAC |
| | | AGACCACGGGGCGGAGATCGTGTACAAGTCGCCAGTGGTGTCTG |
| | | GGGACACGTCTCCACGGCATCTCAGCAATGTCTCCTCCACCGGCA |
| | | GCATCGACATGGTAGACTCGCCCCAGCTCGCCACGCTAGCTGACG |
| | | AGGTGTCTGCCTCCCTGGCCAAGCAGGGTTTGTGATCAGGCCCCT |
| | | GGGGCGGTCAATAATTGTGGAGAGGAGAGAATGAGAGAGTGTGG |
| | | AAAAAAAAAGAATAATGACCCGGCCCCCGCCCTCTGCCCCCAGCT |
| | | GCTCCTCGCAGTTCGGTTAATTGGTTAATCACTTAACCTGCTTTTG |
| | | TCACTCGGCTTTGGCTCGGGACTTCAAAATCAGTGATGGGAGTAA |
| | | GAGCAAATTTCATCTTTCCAAATTGATGGGTGGGCTAGTAATAAA |
| | | ATATTTAAAAAAAAACATTCAAAAACATGGCCACATCCAACATTT |
| | | CCTCAGGCAATTCCTTTTGATTCTTTTTTCTTCCCCCTCCATGTAGA |
| | | AGAGGGAGAAGGAGAGGCTCTGAAAGCTGCTTCTGGGGGATTTC |
| | | AAGGGACTGGGGGTGCCAACCACCTCTGGCCCTGTTGTGGGGGTG |
| | | TCACAGAGGCAGTGGCAGCAACAAAGGATTTGAAACTTGGTGTG |
| | | TTCGTGGAGCCACAGGCAGACGATGTCAACCTTGTGTGAGTGTGA |
| | | CGGGGGTTGGGGTGGGGCGGGAGGCCACGGGGGAGGCCGAGGC |
| | | AGGGGCTGGGCAGAGGGGAGAGGAAGCACAAGAAGTGGGAGTG |
| | | GGAGAGGAAGCCACGTGCTGGAGAGTAGACATCCCCCTCCTTGCC |
| | | GCTGGGAGAGCCAAGGCCTATGCCACCTGCAGCGTCTGAGCGGC |
| | | CGCCTGTCCTTGGTGGCCGGGGGTGGGGGCCTGCTGTGGGTCAGT |
| | | GTGCCACCCTCTGCAGGGCAGCCTGTGGGAGAAGGGACAGCGGG |
| | | TAAAAAGAGAAGGCAAGCTGGCAGGAGGGTGGCACTTCGTGGAT |
| | | GACCTCCTTAGAAAAGACTGACCTTGATGTCTTGAGAGCGCTGGC |
| | | CTCTTCCTCCCTCCCTGCAGGGTAGGGGGCCTGAGTTGAGGGGCT |
| | | TCCCTCTGCTCCACAGAAACCCTGTTTTATTGAGTTCTGAAGGTTG |
| | | GAACTGCTGCCATGATTTTGGCCACTTTGCAGACCTGGGACTTTA |
| | | GGGCTAACCAGTTCTCTTTGTAAGGACTTGTGCCTCTTGGGAGAC |
| | | GTCCACCCGTTTCCAAGCCTGGGCCACTGGCATCTCTGGAGTGTG |
| | | TGGGGGTCTGGGAGGCAGGTCCCGAGCCCCCTGTCCTTCCCACGG |
| | | CCACTGCAGTCACCCCGTCTGCGCCGCTGTGCTGTTGTCTGCCGTG |
| | | AGAGCCCAATCACTGCCTATACCCCTCATCACACGTCACAATGTC |
| | | CCGAATTCCCAGCCTCACCACCCCTTCTCAGTAATGACCCTGGTTG |
| | | GTTGCAGGAGGTACCTACTCCATACTGAGGGTGAAATTAAGGGA |
| | | AGGCAAAGTCCAGGCACAAGAGTGGGACCCCAGCCTCTCACTCTC |
| | | AGTTCCACTCATCCAACTGGGACCCTCACCACGAATCTCATGATC |
| | | TGATTCGGTTCCCTGTCTCCTCCTCCCGTCACAGATGTGAGCCAGG |
| | | GCACTGCTCAGCTGTGACCCTAGGTGTTTCTGCCTTGTTGACATGG |
| | | AGAGAGCCCTTTCCCCTGAGAAGGCCTGGCCCCTTCCTGTGCTGA |
| | | GCCCACAGCAGCAGGCTGGGTGTCTTGGTTGTCAGTGGTGGCACC |
| | | AGGATGGAAGGGCAAGGCACCCAGGGCAGGCCCACAGTCCCGCT |
| | | GTCCCCCACTTGCACCCTAGCTTGTAGCTGCCAACCTCCCAGACA |
| | | GCCCAGCCCGCTGCTCAGCTCCACATGCATAGTATCAGCCCTCCA |
| | | CACCCGACAAAGGGGAACACACCCCCTTGGAAATGGTTCTTTTCC |
| | | CCCAGTCCCAGCTGGAAGCCATGCTGTCTGTTCTGCTGGAGCAGC |
| | | TGAACATATACATAGATGTTGCCCTGCCCTCCCCATCTGCACCCTG |
| | | TTGAGTTGTAGTTGGATTTGTCTGTTTATGCTTGGATTCACCAGAG |
| | | TGACTATGATAGTGAAAAGAAAAAAAAAAAAAAAAAAGGACGC |
| | | ATGTATCTTGAAATGCTTGTAAAGAGGTTTCTAACCCACCCTCAC |
| | | GAGGTGTCTCTCACCCCCACACTGGGACTCGTGTGGCCTGTGTGG |
| | | TGCCACCCTGCTGGGGCCTCCCAAGTTTTGAAAGGCTTTCCTCAG |
| | | CACCTGGGACCCAACAGAGACCAGCTTCTAGCAGCTAAGGAGGC |
| | | CGTTCAGCTGTGACGAAGGCCTGAAGCACAGGATTAGGACTGAA |
| | | GCGATGATGTCCCCTTCCCTACTTCCCCTTGGGGCTCCCTGTGTCA |
| | | GGGCACAGACTAGGTCTTGTGGCTGGTCTGGCTTGCGGCGCGAGG |
| | | ATGGTTCTCTCTGGTCATAGCCCGAAGTCTCATGGCAGTCCCAAA |
| | | GGAGGCTTACAACTCCTGCATCACAAGAAAAAGGAAGCCACTGC |
| | | CAGCTGGGGGGATCTGCAGCTCCCAGAAGCTCCGTGAGCCTCAGC |
| | | CACCCCTCAGACTGGGTTCCTCTCCAAGCTCGCCCTCTGGAGGGG |
| | | CAGCGCAGCCTCCCACCAAGGGCCCTGCGACCACAGCAGGGATT |
| | | GGGATGAATTGCCTGTCCTGGATCTGCTCTAGAGGCCCAAGCTGC |
| | | CTGCCTGAGGAAGGATGACTTGACAAGTCAGGAGACACTGTTCCC |
| | | AAAGCCTTGACCAGAGCACCTCAGCCCGCTGACCTTGCACAAACT |
| | | CCATCTGCTGCCATGAGAAAAGGGAAGCCGCCTTTGCAAAACATT |
| | | GCTGCCTAAAGAAACTCAGCAGCCTCAGGCCCAATTCTGCCACTT |
| | | CTGGTTTGGGTACAGTTAAAGGCAACCCTGAGGGACTTGGCAGTA |
| | | GAAATCCAGGGCCTCCCCTGGGGCTGGCAGCTTCGTGTGCAGCTA |
| | | GAGCTTTACCTGAAAGGAAGTCTCTGGGCCCAGAACTCTCCACCA |
| | | AGAGCCTCCCTGCCGTTCGCTGAGTCCCAGCAATTCTCCTAAGTT |
| | | GAAGGGATCTGAGAAGGAGAAGGAAATGTGGGGTAGATTTGGTG |
| | | GTGGTTAGAGATATGCCCCCCTCATTACTGCCAACAGTTTCGGCT |
| | | GCATTTCTTCACGCACCTCGGTTCCTCTTCCTGAAGTTCTTGTGCC |
| | | CTGCTCTTCAGCACCATGGGCCTTCTTATACGGAAGGCTCTGGGA |
| | | TCTCCCCCTTGTGGGGCAGGCTCTTGGGGCCAGCCTAAGATCATG |
| | | GTTTAGGGTGATCAGTGCTGGCAGATAAATTGAAAAGGCACGCTG |
| | | GCTTGTGATCTTAAATGAGGACAATCCCCCCAGGGCTGGGCACTC |
| | | CTCCCCTCCCCTCACTTCTCCCACCTGCAGAGCCAGTGTCCTTGGG |
| | | TGGGCTAGATAGGATATACTGTATGCCGGCTCCTTCAAGCTGCTG |
| | | ACTCACTTTATCAATAGTTCCATTTAAATTGACTTCAGTGGTGAGA |
| | | CTGTATCCTGTTTGCTATTGCTTGTTGTGCTATGGGGGGAGGGGG |
| | | GAGGAATGTGTAAGATAGTTAACATGGGCAAAGGGAGATCTTGG |
| | | GGTGCAGCACTTAAACTGCCTCGTAACCCTTTTCATGATTTCAACC |
| | | ACATTTGCTAGAGGGAGGGAGCAGCCACGGAGTTAGAGGCCCTT |
| | | GGGGTTTCTCTTTTCCACTGACAGGCTTTCCCAGGCAGCTGGCTAG |
| | | TTCATTCCCTCCCCAGCCAGGTGCAGGCGTAGGAATATGGACATC |
| | | TGGTTGCTTTGGCCTGCTGCCCTCTTTCAGGGGTCCTAAGCCCACA |
| | | ATCATGCCTCCCTAAGACCTTGGCATCCTTCCCTCTAAGCCGTTGG |
| | | CACCTCTGTGCCACCTCTCACACTGGCTCCAGACACACAGCCTGT |
| | | GCTTTTGGAGCTGAGATCACTCGCTTCACCCTCCTCATCTTTGTTC |
| | | TCCAAGTAAAGCCACGAGGTCGGGGCGAGGGCAGAGGTGATCAC |
| | | CTGCGTGTCCCATCTACAGACCTGCAGCTTCATAAAACTTCTGATT |
| | | TCTCTTCAGCTTTGAAAAGGGTTACCCTGGGCACTGGCCTAGAGC |
| | | CTCACCTCCTAATAGACTTAGCCCCATGAGTTTGCCATGTTGAGC |
| | | AGGACTATTTCTGGCACTTGCAAGTCCCATGATTTCTTCGGTAATT |
| | | CTGAGGGTGGGGGGAGGGACATGAAATCATCTTAGCTTAGCTTTC |
| | | TGTCTGTGAATGTCTATATAGTGTATTGTGTGTTTTAACAAATGAT |
| | | TTACACTGACTGTTGCTGTAAAAGTGAATTTGGAAATAAAGTTAT |
| | | TACTCTGATTAAA |
| 43 | 7 | GCAGTCACCGCCACCCACCAGCTCCGGCACCAACAGCAGCGCCG |
| | | CTGCCACCGCCCACCTTCTGCCGCCGCCACCACAGCCACCTTCTCC |
| | | TCCTCCGCTGTCCTCTCCCGTCCTCGCCTCTGTCGACTATCAGGTG |
| | | AACTTTGAACCAGGATGGCTGAGCCCCGCCAGGAGTTCGAAGTG |
| | | ATGGAAGATCACGCTGGGACGTACGGGTTGGGGGACAGGAAAGA |
| | | TCAGGGGGGCTACACCATGCACCAAGACCAAGAGGGTGACACGG |
| | | ACGCTGGCCTGAAAGAATCTCCCCTGCAGACCCCCACTGAGGACG |
| | | GATCTGAGGAACCGGGCTCTGAAACCTCTGATGCTAAGAGCACTC |
| | | CAACAGCGGAAGCTGAAGAAGCAGGCATTGGAGACACCCCCAGC |
| | | CTGGAAGACGAAGCTGCTGGTCACGTGACCCAAGCTCGCATGGTC |
| | | AGTAAAAGCAAAGACGGGACTGGAAGCGATGACAAAAAAGCCA |
| | | AGGGGGCTGATGGTAAAACGAAGATCGCCACACCGCGGGGAGCA |
| | | GCCCCTCCAGGCCAGAAGGGCCAGGCCAACGCCACCAGGATTCC |
| | | AGCAAAAACCCCGCCCGCTCCAAAGACACCACCCAGCTCTGGTG |
| | | AACCTCCAAAATCAGGGGATCGCAGCGGCTACAGCAGCCCCGGC |
| | | TCCCCAGGCACTCCCGGCAGCCGCTCCCGCACCCCGTCCCTTCCA |
| | | ACCCCACCCACCCGGGAGCCCAAGAAGGTGGCAGTGGTCCGTAC |
| | | TCCACCCAAGTCGCCGTCTTCCGCCAAGAGCCGCCTGCAGACAGC |
| | | CCCCGTGCCCATGCCAGACCTGAAGAATGTCAAGTCCAAGATCGG |
| | | CTCCACTGAGAACCTGAAGCACCAGCCGGGAGGCGGGAAGGTGC |
| | | AAATAGTCTACAAACCAGTTGACCTGAGCAAGGTGACCTCCAAGT |
| | | GTGGCTCATTAGGCAACATCCATCATAAACCAGGAGGTGGCCAG |
| | | GTGGAAGTAAAATCTGAGAAGCTTGACTTCAAGGACAGAGTCCA |
| | | GTCGAAGATTGGGTCCCTGGACAATATCACCCACGTCCCTGGCGG |
| | | AGGAAATAAAAAGATTGAAACCCACAAGCTGACCTTCCGCGAGA |
| | | ACGCCAAAGCCAAGACAGACCACGGGGCGGAGATCGTGTACAAG |
| | | TCGCCAGTGGTGTCTGGGGACACGTCTCCACGGCATCTCAGCAAT |
| | | GTCTCCTCCACCGGCAGCATCGACATGGTAGACTCGCCCCAGCTC |
| | | GCCACGCTAGCTGACGAGGTGTCTGCCTCCCTGGCCAAGCAGGGT |
| | | TTGTGATCAGGCCCCTGGGGCGGTCAATAATTGTGGAGAGGAGA |
| | | GAATGAGAGAGTGTGGAAAAAAAAAGAATAATGACCCGGCCCCC |
| | | GCCCTCTGCCCCCAGCTGCTCCTCGCAGTTCGGTTAATTGGTTAAT |
| | | CACTTAACCTGCTTTTGTCACTCGGCTTTGGCTCGGGACTTCAAAA |
| | | TCAGTGATGGGAGTAAGAGCAAATTTCATCTTTCCAAATTGATGG |
| | | GTGGGCTAGTAATAAAATATTTAAAAAAAAACATTCAAAAACAT |
| | | GGCCACATCCAACATTTCCTCAGGCAATTCCTTTTGATTCTTTTTT |
| | | CTTCCCCCTCCATGTAGAAGAGGGAGAAGGAGAGGCTCTGAAAG |
| | | CTGCTTCTGGGGGATTTCAAGGGACTGGGGGTGCCAACCACCTCT |
| | | GGCCCTGTTGTGGGGGTGTCACAGAGGCAGTGGCAGCAACAAAG |
| | | GATTTGAAACTTGGTGTGTTCGTGGAGCCACAGGCAGACGATGTC |
| | | AACCTTGTGTGAGTGTGACGGGGGTTGGGGTGGGGCGGGAGGCC |
| | | ACGGGGGAGGCCGAGGCAGGGGCTGGGCAGAGGGGAGAGGAAG |
| | | CACAAGAAGTGGGAGTGGGAGAGGAAGCCACGTGCTGGAGAGTA |
| | | GACATCCCCCTCCTTGCCGCTGGGAGAGCCAAGGCCTATGCCACC |
| | | TGCAGCGTCTGAGCGGCCGCCTGTCCTTGGTGGCCGGGGGTGGGG |
| | | GCCTGCTGTGGGTCAGTGTGCCACCCTCTGCAGGGCAGCCTGTGG |
| | | GAGAAGGGACAGCGGGTAAAAAGAGAAGGCAAGCTGGCAGGAG |
| | | GGTGGCACTTCGTGGATGACCTCCTTAGAAAAGACTGACCTTGAT |
| | | GTCTTGAGAGCGCTGGCCTCTTCCTCCCTCCCTGCAGGGTAGGGG |
| | | GCCTGAGTTGAGGGGCTTCCCTCTGCTCCACAGAAACCCTGTTTT |
| | | ATTGAGTTCTGAAGGTTGGAACTGCTGCCATGATTTTGGCCACTTT |
| | | GCAGACCTGGGACTTTAGGGCTAACCAGTTCTCTTTGTAAGGACT |
| | | TGTGCCTCTTGGGAGACGTCCACCCGTTTCCAAGCCTGGGCCACT |
| | | GGCATCTCTGGAGTGTGTGGGGGTCTGGGAGGCAGGTCCCGAGCC |
| | | CCCTGTCCTTCCCACGGCCACTGCAGTCACCCCGTCTGCGCCGCTG |
| | | TGCTGTTGTCTGCCGTGAGAGCCCAATCACTGCCTATACCCCTCAT |
| | | CACACGTCACAATGTCCCGAATTCCCAGCCTCACCACCCCTTCTC |
| | | AGTAATGACCCTGGTTGGTTGCAGGAGGTACCTACTCCATACTGA |
| | | GGGTGAAATTAAGGGAAGGCAAAGTCCAGGCACAAGAGTGGGAC |
| | | CCCAGCCTCTCACTCTCAGTTCCACTCATCCAACTGGGACCCTCAC |
| | | CACGAATCTCATGATCTGATTCGGTTCCCTGTCTCCTCCTCCCGTC |
| | | ACAGATGTGAGCCAGGGCACTGCTCAGCTGTGACCCTAGGTGTTT |
| | | CTGCCTTGTTGACATGGAGAGAGCCCTTTCCCCTGAGAAGGCCTG |
| | | GCCCCTTCCTGTGCTGAGCCCACAGCAGCAGGCTGGGTGTCTTGG |
| | | TTGTCAGTGGTGGCACCAGGATGGAAGGGCAAGGCACCCAGGGC |
| | | AGGCCCACAGTCCCGCTGTCCCCCACTTGCACCCTAGCTTGTAGC |
| | | TGCCAACCTCCCAGACAGCCCAGCCCGCTGCTCAGCTCCACATGC |
| | | ATAGTATCAGCCCTCCACACCCGACAAAGGGGAACACACCCCCTT |
| | | GGAAATGGTTCTTTTCCCCCAGTCCCAGCTGGAAGCCATGCTGTC |
| | | TGTTCTGCTGGAGCAGCTGAACATATACATAGATGTTGCCCTGCC |
| | | CTCCCCATCTGCACCCTGTTGAGTTGTAGTTGGATTTGTCTGTTTA |
| | | TGCTTGGATTCACCAGAGTGACTATGATAGTGAAAAGAAAAAAA |
| | | AAAAAAAAAAAGGACGCATGTATCTTGAAATGCTTGTAAAGAGG |
| | | TTTCTAACCCACCCTCACGAGGTGTCTCTCACCCCCACACTGGGA |
| | | CTCGTGTGGCCTGTGTGGTGCCACCCTGCTGGGGCCTCCCAAGTTT |
| | | TGAAAGGCTTTCCTCAGCACCTGGGACCCAACAGAGACCAGCTTC |
| | | TAGCAGCTAAGGAGGCCGTTCAGCTGTGACGAAGGCCTGAAGCA |
| | | CAGGATTAGGACTGAAGCGATGATGTCCCCTTCCCTACTTCCCCTT |
| | | GGGGCTCCCTGTGTCAGGGCACAGACTAGGTCTTGTGGCTGGTCT |
| | | GGCTTGCGGCGCGAGGATGGTTCTCTCTGGTCATAGCCCGAAGTC |
| | | TCATGGCAGTCCCAAAGGAGGCTTACAACTCCTGCATCACAAGAA |
| | | AAAGGAAGCCACTGCCAGCTGGGGGGATCTGCAGCTCCCAGAAG |
| | | CTCCGTGAGCCTCAGCCACCCCTCAGACTGGGTTCCTCTCCAAGC |
| | | TCGCCCTCTGGAGGGGCAGCGCAGCCTCCCACCAAGGGCCCTGCG |
| | | ACCACAGCAGGGATTGGGATGAATTGCCTGTCCTGGATCTGCTCT |
| | | AGAGGCCCAAGCTGCCTGCCTGAGGAAGGATGACTTGACAAGTC |
| | | AGGAGACACTGTTCCCAAAGCCTTGACCAGAGCACCTCAGCCCGC |
| | | TGACCTTGCACAAACTCCATCTGCTGCCATGAGAAAAGGGAAGCC |
| | | GCCTTTGCAAAACATTGCTGCCTAAAGAAACTCAGCAGCCTCAGG |
| | | CCCAATTCTGCCACTTCTGGTTTGGGTACAGTTAAAGGCAACCCT |
| | | GAGGGACTTGGCAGTAGAAATCCAGGGCCTCCCCTGGGGCTGGC |
| | | AGCTTCGTGTGCAGCTAGAGCTTTACCTGAAAGGAAGTCTCTGGG |
| | | CCCAGAACTCTCCACCAAGAGCCTCCCTGCCGTTCGCTGAGTCCC |
| | | AGCAATTCTCCTAAGTTGAAGGGATCTGAGAAGGAGAAGGAAAT |
| | | GTGGGGTAGATTTGGTGGTGGTTAGAGATATGCCCCCCTCATTAC |
| | | TGCCAACAGTTTCGGCTGCATTTCTTCACGCACCTCGGTTCCTCTT |
| | | CCTGAAGTTCTTGTGCCCTGCTCTTCAGCACCATGGGCCTTCTTAT |
| | | ACGGAAGGCTCTGGGATCTCCCCCTTGTGGGGCAGGCTCTTGGGG |
| | | CCAGCCTAAGATCATGGTTTAGGGTGATCAGTGCTGGCAGATAAA |
| | | TTGAAAAGGCACGCTGGCTTGTGATCTTAAATGAGGACAATCCCC |
| | | CCAGGGCTGGGCACTCCTCCCCTCCCCTCACTTCTCCCACCTGCAG |
| | | AGCCAGTGTCCTTGGGTGGGCTAGATAGGATATACTGTATGCCGG |
| | | CTCCTTCAAGCTGCTGACTCACTTTATCAATAGTTCCATTTAAATT |
| | | GACTTCAGTGGTGAGACTGTATCCTGTTTGCTATTGCTTGTTGTGC |
| | | TATGGGGGGAGGGGGGAGGAATGTGTAAGATAGTTAACATGGGC |
| | | AAAGGGAGATCTTGGGGTGCAGCACTTAAACTGCCTCGTAACCCT |
| | | TTTCATGATTTCAACCACATTTGCTAGAGGGAGGGAGCAGCCACG |
| | | GAGTTAGAGGCCCTTGGGGTTTCTCTTTTCCACTGACAGGCTTTCC |
| | | CAGGCAGCTGGCTAGTTCATTCCCTCCCCAGCCAGGTGCAGGCGT |
| | | AGGAATATGGACATCTGGTTGCTTTGGCCTGCTGCCCTCTTTCAGG |
| | | GGTCCTAAGCCCACAATCATGCCTCCCTAAGACCTTGGCATCCTT |
| | | CCCTCTAAGCCGTTGGCACCTCTGTGCCACCTCTCACACTGGCTCC |
| | | AGACACACAGCCTGTGCTTTTGGAGCTGAGATCACTCGCTTCACC |
| | | CTCCTCATCTTTGTTCTCCAAGTAAAGCCACGAGGTCGGGGCGAG |
| | | GGCAGAGGTGATCACCTGCGTGTCCCATCTACAGACCTGCAGCTT |
| | | CATAAAACTTCTGATTTCTCTTCAGCTTTGAAAAGGGTTACCCTGG |
| | | GCACTGGCCTAGAGCCTCACCTCCTAATAGACTTAGCCCCATGAG |
| | | TTTGCCATGTTGAGCAGGACTATTTCTGGCACTTGCAAGTCCCATG |
| | | ATTTCTTCGGTAATTCTGAGGGTGGGGGGAGGGACATGAAATCAT |
| | | CTTAGCTTAGCTTTCTGTCTGTGAATGTCTATATAGTGTATTGTGT |
| | | GTTTTAACAAATGATTTACACTGACTGTTGCTGTAAAAGTGAATTT |
| | | GGAAATAAAGTTATTACTCTGATTAAA |
| 44 | 8 | GCAGTCACCGCCACCCACCAGCTCCGGCACCAACAGCAGCGCCG |
| | | CTGCCACCGCCCACCTTCTGCCGCCGCCACCACAGCCACCTTCTCC |
| | | TCCTCCGCTGTCCTCTCCCGTCCTCGCCTCTGTCGACTATCAGGTG |
| | | AACTTTGAACCAGGATGGCTGAGCCCCGCCAGGAGTTCGAAGTG |
| | | ATGGAAGATCACGCTGGGACGTACGGGTTGGGGGACAGGAAAGA |
| | | TCAGGGGGGCTACACCATGCACCAAGACCAAGAGGGTGACACGG |
| | | ACGCTGGCCTGAAAGAATCTCCCCTGCAGACCCCCACTGAGGACG |
| | | GATCTGAGGAACCGGGCTCTGAAACCTCTGATGCTAAGAGCACTC |
| | | CAACAGCGGAAGATGTGACAGCACCCTTAGTGGATGAGGGAGCT |
| | | CCCGGCAAGCAGGCTGCCGCGCAGCCCCACACGGAGATCCCAGA |
| | | AGGAACCACAGCTGAAGAAGCAGGCATTGGAGACACCCCCAGCC |
| | | TGGAAGACGAAGCTGCTGGTCACGTGACCCAAGCTCGCATGGTCA |
| | | GTAAAAGCAAAGACGGGACTGGAAGCGATGACAAAAAAGCCAA |
| | | GGGGGCTGATGGTAAAACGAAGATCGCCACACCGCGGGGAGCAG |
| | | CCCCTCCAGGCCAGAAGGGCCAGGCCAACGCCACCAGGATTCCA |
| | | GCAAAAACCCCGCCCGCTCCAAAGACACCACCCAGCTCTGGTGA |
| | | ACCTCCAAAATCAGGGGATCGCAGCGGCTACAGCAGCCCCGGCT |
| | | CCCCAGGCACTCCCGGCAGCCGCTCCCGCACCCCGTCCCTTCCAA |
| | | CCCCACCCACCCGGGAGCCCAAGAAGGTGGCAGTGGTCCGTACTC |
| | | CACCCAAGTCGCCGTCTTCCGCCAAGAGCCGCCTGCAGACAGCCC |
| | | CCGTGCCCATGCCAGACCTGAAGAATGTCAAGTCCAAGATCGGCT |
| | | CCACTGAGAACCTGAAGCACCAGCCGGGAGGCGGGAAGGTGCAA |
| | | ATAGTCTACAAACCAGTTGACCTGAGCAAGGTGACCTCCAAGTGT |
| | | GGCTCATTAGGCAACATCCATCATAAACCAGGAGGTGGCCAGGT |
| | | GGAAGTAAAATCTGAGAAGCTTGACTTCAAGGACAGAGTCCAGT |
| | | CGAAGATTGGGTCCCTGGACAATATCACCCACGTCCCTGGCGGAG |
| | | GAAATAAAAAGATTGAAACCCACAAGCTGACCTTCCGCGAGAAC |
| | | GCCAAAGCCAAGACAGACCACGGGGCGGAGATCGTGTACAAGTC |
| | | GCCAGTGGTGTCTGGGGACACGTCTCCACGGCATCTCAGCAATGT |
| | | CTCCTCCACCGGCAGCATCGACATGGTAGACTCGCCCCAGCTCGC |
| | | CACGCTAGCTGACGAGGTGTCTGCCTCCCTGGCCAAGCAGGGTTT |
| | | GTGATCAGGCCCCTGGGGCGGTCAATAATTGTGGAGAGGAGAGA |
| | | ATGAGAGAGTGTGGAAAAAAAAAGAATAATGACCCGGCCCCCGC |
| | | CCTCTGCCCCCAGCTGCTCCTCGCAGTTCGGTTAATTGGTTAATCA |
| | | CTTAACCTGCTTTTGTCACTCGGCTTTGGCTCGGGACTTCAAAATC |
| | | AGTGATGGGAGTAAGAGCAAATTTCATCTTTCCAAATTGATGGGT |
| | | GGGCTAGTAATAAAATATTTAAAAAAAAACATTCAAAAACATGG |
| | | CCACATCCAACATTTCCTCAGGCAATTCCTTTTGATTCTTTTTTCTT |
| | | CCCCCTCCATGTAGAAGAGGGAGAAGGAGAGGCTCTGAAAGCTG |
| | | CTTCTGGGGGATTTCAAGGGACTGGGGGTGCCAACCACCTCTGGC |
| | | CCTGTTGTGGGGGTGTCACAGAGGCAGTGGCAGCAACAAAGGAT |
| | | TTGAAACTTGGTGTGTTCGTGGAGCCACAGGCAGACGATGTCAAC |
| | | CTTGTGTGAGTGTGACGGGGGTTGGGGTGGGGCGGGAGGCCACG |
| | | GGGGAGGCCGAGGCAGGGGCTGGGCAGAGGGGAGAGGAAGCAC |
| | | AAGAAGTGGGAGTGGGAGAGGAAGCCACGTGCTGGAGAGTAGAC |
| | | ATCCCCCTCCTTGCCGCTGGGAGAGCCAAGGCCTATGCCACCTGC |
| | | AGCGTCTGAGCGGCCGCCTGTCCTTGGTGGCCGGGGGTGGGGGCC |
| | | TGCTGTGGGTCAGTGTGCCACCCTCTGCAGGGCAGCCTGTGGGAG |
| | | AAGGGACAGCGGGTAAAAAGAGAAGGCAAGCTGGCAGGAGGGT |
| | | GGCACTTCGTGGATGACCTCCTTAGAAAAGACTGACCTTGATGTC |
| | | TTGAGAGCGCTGGCCTCTTCCTCCCTCCCTGCAGGGTAGGGGGCC |
| | | TGAGTTGAGGGGCTTCCCTCTGCTCCACAGAAACCCTGTTTTATTG |
| | | AGTTCTGAAGGTTGGAACTGCTGCCATGATTTTGGCCACTTTGCA |
| | | GACCTGGGACTTTAGGGCTAACCAGTTCTCTTTGTAAGGACTTGT |
| | | GCCTCTTGGGAGACGTCCACCCGTTTCCAAGCCTGGGCCACTGGC |
| | | ATCTCTGGAGTGTGTGGGGGTCTGGGAGGCAGGTCCCGAGCCCCC |
| | | TGTCCTTCCCACGGCCACTGCAGTCACCCCGTCTGCGCCGCTGTGC |
| | | TGTTGTCTGCCGTGAGAGCCCAATCACTGCCTATACCCCTCATCAC |
| | | ACGTCACAATGTCCCGAATTCCCAGCCTCACCACCCCTTCTCAGT |
| | | AATGACCCTGGTTGGTTGCAGGAGGTACCTACTCCATACTGAGGG |
| | | TGAAATTAAGGGAAGGCAAAGTCCAGGCACAAGAGTGGGACCCC |
| | | AGCCTCTCACTCTCAGTTCCACTCATCCAACTGGGACCCTCACCAC |
| | | GAATCTCATGATCTGATTCGGTTCCCTGTCTCCTCCTCCCGTCACA |
| | | GATGTGAGCCAGGGCACTGCTCAGCTGTGACCCTAGGTGTTTCTG |
| | | CCTTGTTGACATGGAGAGAGCCCTTTCCCCTGAGAAGGCCTGGCC |
| | | CCTTCCTGTGCTGAGCCCACAGCAGCAGGCTGGGTGTCTTGGTTG |
| | | TCAGTGGTGGCACCAGGATGGAAGGGCAAGGCACCCAGGGCAGG |
| | | CCCACAGTCCCGCTGTCCCCCACTTGCACCCTAGCTTGTAGCTGCC |
| | | AACCTCCCAGACAGCCCAGCCCGCTGCTCAGCTCCACATGCATAG |
| | | TATCAGCCCTCCACACCCGACAAAGGGGAACACACCCCCTTGGAA |
| | | ATGGTTCTTTTCCCCCAGTCCCAGCTGGAAGCCATGCTGTCTGTTC |
| | | TGCTGGAGCAGCTGAACATATACATAGATGTTGCCCTGCCCTCCC |
| | | CATCTGCACCCTGTTGAGTTGTAGTTGGATTTGTCTGTTTATGCTT |
| | | GGATTCACCAGAGTGACTATGATAGTGAAAAGAAAAAAAAAAAA |
| | | AAAAAAGGACGCATGTATCTTGAAATGCTTGTAAAGAGGTTTCTA |
| | | ACCCACCCTCACGAGGTGTCTCTCACCCCCACACTGGGACTCGTG |
| | | TGGCCTGTGTGGTGCCACCCTGCTGGGGCCTCCCAAGTTTTGAAA |
| | | GGCTTTCCTCAGCACCTGGGACCCAACAGAGACCAGCTTCTAGCA |
| | | GCTAAGGAGGCCGTTCAGCTGTGACGAAGGCCTGAAGCACAGGA |
| | | TTAGGACTGAAGCGATGATGTCCCCTTCCCTACTTCCCCTTGGGGC |
| | | TCCCTGTGTCAGGGCACAGACTAGGTCTTGTGGCTGGTCTGGCTT |
| | | GCGGCGCGAGGATGGTTCTCTCTGGTCATAGCCCGAAGTCTCATG |
| | | GCAGTCCCAAAGGAGGCTTACAACTCCTGCATCACAAGAAAAAG |
| | | GAAGCCACTGCCAGCTGGGGGGATCTGCAGCTCCCAGAAGCTCC |
| | | GTGAGCCTCAGCCACCCCTCAGACTGGGTTCCTCTCCAAGCTCGC |
| | | CCTCTGGAGGGGCAGCGCAGCCTCCCACCAAGGGCCCTGCGACC |
| | | ACAGCAGGGATTGGGATGAATTGCCTGTCCTGGATCTGCTCTAGA |
| | | GGCCCAAGCTGCCTGCCTGAGGAAGGATGACTTGACAAGTCAGG |
| | | AGACACTGTTCCCAAAGCCTTGACCAGAGCACCTCAGCCCGCTGA |
| | | CCTTGCACAAACTCCATCTGCTGCCATGAGAAAAGGGAAGCCGCC |
| | | TTTGCAAAACATTGCTGCCTAAAGAAACTCAGCAGCCTCAGGCCC |
| | | AATTCTGCCACTTCTGGTTTGGGTACAGTTAAAGGCAACCCTGAG |
| | | GGACTTGGCAGTAGAAATCCAGGGCCTCCCCTGGGGCTGGCAGCT |
| | | TCGTGTGCAGCTAGAGCTTTACCTGAAAGGAAGTCTCTGGGCCCA |
| | | GAACTCTCCACCAAGAGCCTCCCTGCCGTTCGCTGAGTCCCAGCA |
| | | ATTCTCCTAAGTTGAAGGGATCTGAGAAGGAGAAGGAAATGTGG |
| | | GGTAGATTTGGTGGTGGTTAGAGATATGCCCCCCTCATTACTGCC |
| | | AACAGTTTCGGCTGCATTTCTTCACGCACCTCGGTTCCTCTTCCTG |
| | | AAGTTCTTGTGCCCTGCTCTTCAGCACCATGGGCCTTCTTATACGG |
| | | AAGGCTCTGGGATCTCCCCCTTGTGGGGCAGGCTCTTGGGGCCAG |
| | | CCTAAGATCATGGTTTAGGGTGATCAGTGCTGGCAGATAAATTGA |
| | | AAAGGCACGCTGGCTTGTGATCTTAAATGAGGACAATCCCCCCAG |
| | | GGCTGGGCACTCCTCCCCTCCCCTCACTTCTCCCACCTGCAGAGCC |
| | | AGTGTCCTTGGGTGGGCTAGATAGGATATACTGTATGCCGGCTCC |
| | | TTCAAGCTGCTGACTCACTTTATCAATAGTTCCATTTAAATTGACT |
| | | TCAGTGGTGAGACTGTATCCTGTTTGCTATTGCTTGTTGTGCTATG |
| | | GGGGGAGGGGGGAGGAATGTGTAAGATAGTTAACATGGGCAAAG |
| | | GGAGATCTTGGGGTGCAGCACTTAAACTGCCTCGTAACCCTTTTC |
| | | ATGATTTCAACCACATTTGCTAGAGGGAGGGAGCAGCCACGGAG |
| | | TTAGAGGCCCTTGGGGTTTCTCTTTTCCACTGACAGGCTTTCCCAG |
| | | GCAGCTGGCTAGTTCATTCCCTCCCCAGCCAGGTGCAGGCGTAGG |
| | | AATATGGACATCTGGTTGCTTTGGCCTGCTGCCCTCTTTCAGGGGT |
| | | CCTAAGCCCACAATCATGCCTCCCTAAGACCTTGGCATCCTTCCCT |
| | | CTAAGCCGTTGGCACCTCTGTGCCACCTCTCACACTGGCTCCAGA |
| | | CACACAGCCTGTGCTTTTGGAGCTGAGATCACTCGCTTCACCCTCC |
| | | TCATCTTTGTTCTCCAAGTAAAGCCACGAGGTCGGGGCGAGGGCA |
| | | GAGGTGATCACCTGCGTGTCCCATCTACAGACCTGCAGCTTCATA |
| | | AAACTTCTGATTTCTCTTCAGCTTTGAAAAGGGTTACCCTGGGCAC |
| | | TGGCCTAGAGCCTCACCTCCTAATAGACTTAGCCCCATGAGTTTG |
| | | CCATGTTGAGCAGGACTATTTCTGGCACTTGCAAGTCCCATGATTT |
| | | CTTCGGTAATTCTGAGGGTGGGGGGAGGGACATGAAATCATCTTA |
| | | GCTTAGCTTTCTGTCTGTGAATGTCTATATAGTGTATTGTGTGTTT |
| | | TAACAAATGATTTACACTGACTGTTGCTGTAAAAGTGAATTTGGA |
| | | AATAAAGTTATTACTCTGATTAAA |
| 45 | 9 | GCAGTCACCGCCACCCACCAGCTCCGGCACCAACAGCAGCGCCG |
| | | CTGCCACCGCCCACCTTCTGCCGCCGCCACCACAGCCACCTTCTCC |
| | | TCCTCCGCTGTCCTCTCCCGTCCTCGCCTCTGTCGACTATCAGGTG |
| | | AACTTTGAACCAGGATGGCTGAGCCCCGCCAGGAGTTCGAAGTG |
| | | ATGGAAGATCACGCTGGGACGTACGGGTTGGGGGACAGGAAAGA |
| | | TCAGGGGGGCTACACCATGCACCAAGACCAAGAGGGTGACACGG |
| | | ACGCTGGCCTGAAAGAATCTCCCCTGCAGACCCCCACTGAGGACG |
| | | GATCTGAGGAACCGGGCTCTGAAACCTCTGATGCTAAGAGCACTC |
| | | CAACAGCGGAAGCTGAAGAAGCAGGCATTGGAGACACCCCCAGC |
| | | CTGGAAGACGAAGCTGCTGGTCACGTGACCCAAGAGGAGTTGAG |
| | | AGTTCCGGGCCGGCAGAGGAAGGCGCCTGAAAGGCCCCTGGCCA |
| | | ATGAGATTAGCGCCCACGTCCAGCCTGGACCCTGCGGAGAGGCCT |
| | | CTGGGGTCTCTGGGCCGTGCCTCGGGGAGAAAGAGCCAGAAGCT |
| | | CCCGTCCCGCTGACCGCGAGCCTTCCTCAGCACCGTCCCGTTTGCC |
| | | CAGCGCCTCCTCCAACAGGAGGCCCTCAGGAGCCCTCCCTGGAGT |
| | | GGGGACAAAAAGGCGGGGACTGGGCCGAGAAGGGTCCGGCCTTT |
| | | CCGAAGCCCGCCACCACTGCGTATCTCCACACAGAGCCTGAAAGT |
| | | GGTAAGGTGGTCCAGGAAGGCTTCCTCCGAGAGCCAGGCCCCCC |
| | | AGGTCTGAGCCACCAGCTCATGTCCGGCATGCCTGGGGCTCCCCT |
| | | CCTGCCTGAGGGCCCCAGAGAGGCCACACGCCAACCTTCGGGGA |
| | | CAGGACCTGAGGACACAGAGGGCGGCCGCCACGCCCCTGAGCTG |
| | | CTCAAGCACCAGCTTCTAGGAGACCTGCACCAGGAGGGGCCGCC |
| | | GCTGAAGGGGGCAGGGGGCAAAGAGAGGCCGGGGAGCAAGGAG |
| | | GAGGTGGATGAAGACCGCGACGTCGATGAGTCCTCCCCCCAAGA |
| | | CTCCCCTCCCTCCAAGGCCTCCCCAGCCCAAGATGGGCGGCCTCC |
| | | CCAGACAGCCGCCAGAGAAGCCACCAGCATCCCAGGCTTCCCAG |
| | | CGGAGGGTGCCATCCCCCTCCCTGTGGATTTCCTCTCCAAAGTTTC |
| | | CACAGAGATCCCAGCCTCAGAGCCCGACGGGCCCAGTGTAGGGC |
| | | GGGCCAAAGGGCAGGATGCCCCCCTGGAGTTCACGTTTCACGTGG |
| | | AAATCACACCCAACGTGCAGAAGGAGCAGGCGCACTCGGAGGAG |
| | | CATTTGGGAAGGGCTGCATTTCCAGGGGCCCCTGGAGAGGGGCC |
| | | AGAGGCCCGGGGCCCCTCTTTGGGAGAGGACACAAAAGAGGCTG |
| | | ACCTTCCAGAGCCCTCTGAAAAGCAGCCTGCTGCTGCTCCGCGGG |
| | | GGAAGCCCGTCAGCCGGGTCCCTCAACTCAAAGCTCGCATGGTCA |
| | | GTAAAAGCAAAGACGGGACTGGAAGCGATGACAAAAAAGCCAA |
| | | GACATCCACACGTTCCTCTGCTAAAACCTTGAAAAATAGGCCTTG |
| | | CCTTAGCCCCAAACACCCCACTCCTGGTAGCTCAGACCCTCTGAT |
| | | CCAACCCTCCAGCCCTGCTGTGTGCCCAGAGCCACCTTCCTCTCCT |
| | | AAATACGTCTCTTCTGTCACTTCCCGAACTGGCAGTTCTGGAGCA |
| | | AAGGAGATGAAACTCAAGGGGGCTGATGGTAAAACGAAGATCGC |
| | | CACACCGCGGGGAGCAGCCCCTCCAGGCCAGAAGGGCCAGGCCA |
| | | ACGCCACCAGGATTCCAGCAAAAACCCCGCCCGCTCCAAAGACA |
| | | CCACCCAGCTCTGGTGAACCTCCAAAATCAGGGGATCGCAGCGGC |
| | | TACAGCAGCCCCGGCTCCCCAGGCACTCCCGGCAGCCGCTCCCGC |
| | | ACCCCGTCCCTTCCAACCCCACCCACCCGGGAGCCCAAGAAGGTG |
| | | GCAGTGGTCCGTACTCCACCCAAGTCGCCGTCTTCCGCCAAGAGC |
| | | CGCCTGCAGACAGCCCCCGTGCCCATGCCAGACCTGAAGAATGTC |
| | | AAGTCCAAGATCGGCTCCACTGAGAACCTGAAGCACCAGCCGGG |
| | | AGGCGGGAAGGTGCAGATAATTAATAAGAAGCTGGATCTTAGCA |
| | | ACGTCCAGTCCAAGTGTGGCTCAAAGGATAATATCAAACACGTCC |
| | | CGGGAGGCGGCAGTGTGCAAATAGTCTACAAACCAGTTGACCTG |
| | | AGCAAGGTGACCTCCAAGTGTGGCTCATTAGGCAACATCCATCAT |
| | | AAACCAGGAGGTGGCCAGGTGGAAGTAAAATCTGAGAAGCTTGA |
| | | CTTCAAGGACAGAGTCCAGTCGAAGATTGGGTCCCTGGACAATAT |
| | | CACCCACGTCCCTGGCGGAGGAAATAAAAAGATTGAAACCCACA |
| | | AGCTGACCTTCCGCGAGAACGCCAAAGCCAAGACAGACCACGGG |
| | | GCGGAGATCGTGTACAAGTCGCCAGTGGTGTCTGGGGACACGTCT |
| | | CCACGGCATCTCAGCAATGTCTCCTCCACCGGCAGCATCGACATG |
| | | GTAGACTCGCCCCAGCTCGCCACGCTAGCTGACGAGGTGTCTGCC |
| | | TCCCTGGCCAAGCAGGGTTTGTGATCAGGCCCCTGGGGCGGTCAA |
| | | TAATTGTGGAGAGGAGAGAATGAGAGAGTGTGGAAAAAAAAAG |
| | | AATAATGACCCGGCCCCCGCCCTCTGCCCCCAGCTGCTCCTCGCA |
| | | GTTCGGTTAATTGGTTAATCACTTAACCTGCTTTTGTCACTCGGCT |
| | | TTGGCTCGGGACTTCAAAATCAGTGATGGGAGTAAGAGCAAATTT |
| | | CATCTTTCCAAATTGATGGGTGGGCTAGTAATAAAATATTTAAAA |
| | | AAAAACATTCAAAAACATGGCCACATCCAACATTTCCTCAGGCAA |
| | | TTCCTTTTGATTCTTTTTTCTTCCCCCTCCATGTAGAAGAGGGAGA |
| | | AGGAGAGGCTCTGAAAGCTGCTTCTGGGGGATTTCAAGGGACTG |
| | | GGGGTGCCAACCACCTCTGGCCCTGTTGTGGGGGTGTCACAGAGG |
| | | CAGTGGCAGCAACAAAGGATTTGAAACTTGGTGTGTTCGTGGAGC |
| | | CACAGGCAGACGATGTCAACCTTGTGTGAGTGTGACGGGGGTTGG |
| | | GGTGGGGCGGGAGGCCACGGGGGAGGCCGAGGCAGGGGCTGGG |
| | | CAGAGGGGAGAGGAAGCACAAGAAGTGGGAGTGGGAGAGGAAG |
| | | CCACGTGCTGGAGAGTAGACATCCCCCTCCTTGCCGCTGGGAGAG |
| | | CCAAGGCCTATGCCACCTGCAGCGTCTGAGCGGCCGCCTGTCCTT |
| | | GGTGGCCGGGGGTGGGGGCCTGCTGTGGGTCAGTGTGCCACCCTC |
| | | TGCAGGGCAGCCTGTGGGAGAAGGGACAGCGGGTAAAAAGAGA |
| | | AGGCAAGCTGGCAGGAGGGTGGCACTTCGTGGATGACCTCCTTAG |
| | | AAAAGACTGACCTTGATGTCTTGAGAGCGCTGGCCTCTTCCTCCC |
| | | TCCCTGCAGGGTAGGGGGCCTGAGTTGAGGGGCTTCCCTCTGCTC |
| | | CACAGAAACCCTGTTTTATTGAGTTCTGAAGGTTGGAACTGCTGC |
| | | CATGATTTTGGCCACTTTGCAGACCTGGGACTTTAGGGCTAACCA |
| | | GTTCTCTTTGTAAGGACTTGTGCCTCTTGGGAGACGTCCACCCGTT |
| | | TCCAAGCCTGGGCCACTGGCATCTCTGGAGTGTGTGGGGGTCTGG |
| | | GAGGCAGGTCCCGAGCCCCCTGTCCTTCCCACGGCCACTGCAGTC |
| | | ACCCCGTCTGCGCCGCTGTGCTGTTGTCTGCCGTGAGAGCCCAAT |
| | | CACTGCCTATACCCCTCATCACACGTCACAATGTCCCGAATTCCC |
| | | AGCCTCACCACCCCTTCTCAGTAATGACCCTGGTTGGTTGCAGGA |
| | | GGTACCTACTCCATACTGAGGGTGAAATTAAGGGAAGGCAAAGT |
| | | CCAGGCACAAGAGTGGGACCCCAGCCTCTCACTCTCAGTTCCACT |
| | | CATCCAACTGGGACCCTCACCACGAATCTCATGATCTGATTCGGT |
| | | TCCCTGTCTCCTCCTCCCGTCACAGATGTGAGCCAGGGCACTGCTC |
| | | AGCTGTGACCCTAGGTGTTTCTGCCTTGTTGACATGGAGAGAGCC |
| | | CTTTCCCCTGAGAAGGCCTGGCCCCTTCCTGTGCTGAGCCCACAG |
| | | CAGCAGGCTGGGTGTCTTGGTTGTCAGTGGTGGCACCAGGATGGA |
| | | AGGGCAAGGCACCCAGGGCAGGCCCACAGTCCCGCTGTCCCCCA |
| | | CTTGCACCCTAGCTTGTAGCTGCCAACCTCCCAGACAGCCCAGCC |
| | | CGCTGCTCAGCTCCACATGCATAGTATCAGCCCTCCACACCCGAC |
| | | AAAGGGGAACACACCCCCTTGGAAATGGTTCTTTTCCCCCAGTCC |
| | | CAGCTGGAAGCCATGCTGTCTGTTCTGCTGGAGCAGCTGAACATA |
| | | TACATAGATGTTGCCCTGCCCTCCCCATCTGCACCCTGTTGAGTTG |
| | | TAGTTGGATTTGTCTGTTTATGCTTGGATTCACCAGAGTGACTATG |
| | | ATAGTGAAAAGAAAAAAAAAAAAAAAAAAGGACGCATGTATCTT |
| | | GAAATGCTTGTAAAGAGGTTTCTAACCCACCCTCACGAGGTGTCT |
| | | CTCACCCCCACACTGGGACTCGTGTGGCCTGTGTGGTGCCACCCT |
| | | GCTGGGGCCTCCCAAGTTTTGAAAGGCTTTCCTCAGCACCTGGGA |
| | | CCCAACAGAGACCAGCTTCTAGCAGCTAAGGAGGCCGTTCAGCTG |
| | | TGACGAAGGCCTGAAGCACAGGATTAGGACTGAAGCGATGATGT |
| | | CCCCTTCCCTACTTCCCCTTGGGGCTCCCTGTGTCAGGGCACAGAC |
| | | TAGGTCTTGTGGCTGGTCTGGCTTGCGGCGCGAGGATGGTTCTCT |
| | | CTGGTCATAGCCCGAAGTCTCATGGCAGTCCCAAAGGAGGCTTAC |
| | | AACTCCTGCATCACAAGAAAAAGGAAGCCACTGCCAGCTGGGGG |
| | | GATCTGCAGCTCCCAGAAGCTCCGTGAGCCTCAGCCACCCCTCAG |
| | | ACTGGGTTCCTCTCCAAGCTCGCCCTCTGGAGGGGCAGCGCAGCC |
| | | TCCCACCAAGGGCCCTGCGACCACAGCAGGGATTGGGATGAATT |
| | | GCCTGTCCTGGATCTGCTCTAGAGGCCCAAGCTGCCTGCCTGAGG |
| | | AAGGATGACTTGACAAGTCAGGAGACACTGTTCCCAAAGCCTTGA |
| | | CCAGAGCACCTCAGCCCGCTGACCTTGCACAAACTCCATCTGCTG |
| | | CCATGAGAAAAGGGAAGCCGCCTTTGCAAAACATTGCTGCCTAA |
| | | AGAAACTCAGCAGCCTCAGGCCCAATTCTGCCACTTCTGGTTTGG |
| | | GTACAGTTAAAGGCAACCCTGAGGGACTTGGCAGTAGAAATCCA |
| | | GGGCCTCCCCTGGGGCTGGCAGCTTCGTGTGCAGCTAGAGCTTTA |
| | | CCTGAAAGGAAGTCTCTGGGCCCAGAACTCTCCACCAAGAGCCTC |
| | | CCTGCCGTTCGCTGAGTCCCAGCAATTCTCCTAAGTTGAAGGGAT |
| | | CTGAGAAGGAGAAGGAAATGTGGGGTAGATTTGGTGGTGGTTAG |
| | | AGATATGCCCCCCTCATTACTGCCAACAGTTTCGGCTGCATTTCTT |
| | | CACGCACCTCGGTTCCTCTTCCTGAAGTTCTTGTGCCCTGCTCTTC |
| | | AGCACCATGGGCCTTCTTATACGGAAGGCTCTGGGATCTCCCCCT |
| | | TGTGGGGCAGGCTCTTGGGGCCAGCCTAAGATCATGGTTTAGGGT |
| | | GATCAGTGCTGGCAGATAAATTGAAAAGGCACGCTGGCTTGTGAT |
| | | CTTAAATGAGGACAATCCCCCCAGGGCTGGGCACTCCTCCCCTCC |
| | | CCTCACTTCTCCCACCTGCAGAGCCAGTGTCCTTGGGTGGGCTAG |
| | | ATAGGATATACTGTATGCCGGCTCCTTCAAGCTGCTGACTCACTTT |
| | | ATCAATAGTTCCATTTAAATTGACTTCAGTGGTGAGACTGTATCCT |
| | | GTTTGCTATTGCTTGTTGTGCTATGGGGGGAGGGGGGAGGAATGT |
| | | GTAAGATAGTTAACATGGGCAAAGGGAGATCTTGGGGTGCAGCA |
| | | CTTAAACTGCCTCGTAACCCTTTTCATGATTTCAACCACATTTGCT |
| | | AGAGGGAGGGAGCAGCCACGGAGTTAGAGGCCCTTGGGGTTTCT |
| | | CTTTTCCACTGACAGGCTTTCCCAGGCAGCTGGCTAGTTCATTCCC |
| | | TCCCCAGCCAGGTGCAGGCGTAGGAATATGGACATCTGGTTGCTT |
| | | TGGCCTGCTGCCCTCTTTCAGGGGTCCTAAGCCCACAATCATGCCT |
| | | CCCTAAGACCTTGGCATCCTTCCCTCTAAGCCGTTGGCACCTCTGT |
| | | GCCACCTCTCACACTGGCTCCAGACACACAGCCTGTGCTTTTGGA |
| | | GCTGAGATCACTCGCTTCACCCTCCTCATCTTTGTTCTCCAAGTAA |
| | | AGCCACGAGGTCGGGGCGAGGGCAGAGGTGATCACCTGCGTGTC |
| | | CCATCTACAGACCTGCAGCTTCATAAAACTTCTGATTTCTCTTCAG |
| | | CTTTGAAAAGGGTTACCCTGGGCACTGGCCTAGAGCCTCACCTCC |
| | | TAATAGACTTAGCCCCATGAGTTTGCCATGTTGAGCAGGACTATT |
| | | TCTGGCACTTGCAAGTCCCATGATTTCTTCGGTAATTCTGAGGGTG |
| | | GGGGGAGGGACATGAAATCATCTTAGCTTAGCTTTCTGTCTGTGA |
| | | ATGTCTATATAGTGTATTGTGTGTTTTAACAAATGATTTACACTGA |
| | | CTGTTGCTGTAAAAGTGAATTTGGAAATAAAGTTATTACTCTGAT |
| | | TAAA |
| 46 | 10 | GCAGTCACCGCCACCCACCAGCTCCGGCACCAACAGCAGCGCCG |
| | | CTGCCACCGCCCACCTTCTGCCGCCGCCACCACAGCCACCTTCTCC |
| | | TCCTCCGCTGTCCTCTCCCGTCCTCGCCTCTGTCGACTATCAGGTG |
| | | AACTTTGAACCAGGATGGCTGAGCCCCGCCAGGAGTTCGAAGTG |
| | | ATGGAAGATCACGCTGGGACGTACGGGTTGGGGGACAGGAAAGA |
| | | TCAGGGGGGCTACACCATGCACCAAGACCAAGAGGGTGACACGG |
| | | ACGCTGGCCTGAAAGAATCTCCCCTGCAGACCCCCACTGAGGACG |
| | | GATCTGAGGAACCGGGCTCTGAAACCTCTGATGCTAAGAGCACTC |
| | | CAACAGCGGAAGCTGAAGAAGCAGGCATTGGAGACACCCCCAGC |
| | | CTGGAAGACGAAGCTGCTGGTCACGTGACCCAAGAGGAGTTGAG |
| | | AGTTCCGGGCCGGCAGAGGAAGGCGCCTGAAAGGCCCCTGGCCA |
| | | ATGAGATTAGCGCCCACGTCCAGCCTGGACCCTGCGGAGAGGCCT |
| | | CTGGGGTCTCTGGGCCGTGCCTCGGGGAGAAAGAGCCAGAAGCT |
| | | CCCGTCCCGCTGACCGCGAGCCTTCCTCAGCACCGTCCCGTTTGCC |
| | | CAGCGCCTCCTCCAACAGGAGGCCCTCAGGAGCCCTCCCTGGAGT |
| | | GGGGACAAAAAGGCGGGGACTGGGCCGAGAAGGGTCCGGCCTTT |
| | | CCGAAGCCCGCCACCACTGCGTATCTCCACACAGAGCCTGAAAGT |
| | | GGTAAGGTGGTCCAGGAAGGCTTCCTCCGAGAGCCAGGCCCCCC |
| | | AGGTCTGAGCCACCAGCTCATGTCCGGCATGCCTGGGGCTCCCCT |
| | | CCTGCCTGAGGGCCCCAGAGAGGCCACACGCCAACCTTCGGGGA |
| | | CAGGACCTGAGGACACAGAGGGCGGCCGCCACGCCCCTGAGCTG |
| | | CTCAAGCACCAGCTTCTAGGAGACCTGCACCAGGAGGGGCCGCC |
| | | GCTGAAGGGGGCAGGGGGCAAAGAGAGGCCGGGGAGCAAGGAG |
| | | GAGGTGGATGAAGACCGCGACGTCGATGAGTCCTCCCCCCAAGA |
| | | CTCCCCTCCCTCCAAGGCCTCCCCAGCCCAAGATGGGCGGCCTCC |
| | | CCAGACAGCCGCCAGAGAAGCCACCAGCATCCCAGGCTTCCCAG |
| | | CGGAGGGTGCCATCCCCCTCCCTGTGGATTTCCTCTCCAAAGTTTC |
| | | CACAGAGATCCCAGCCTCAGAGCCCGACGGGCCCAGTGTAGGGC |
| | | GGGCCAAAGGGCAGGATGCCCCCCTGGAGTTCACGTTTCACGTGG |
| | | AAATCACACCCAACGTGCAGAAGGAGCAGGCGCACTCGGAGGAG |
| | | CATTTGGGAAGGGCTGCATTTCCAGGGGCCCCTGGAGAGGGGCC |
| | | AGAGGCCCGGGGCCCCTCTTTGGGAGAGGACACAAAAGAGGCTG |
| | | ACCTTCCAGAGCCCTCTGAAAAGCAGCCTGCTGCTGCTCCGCGGG |
| | | GGAAGCCCGTCAGCCGGGTCCCTCAACTCAAAGCTCGCATGGTCA |
| | | GTAAAAGCAAAGACGGGACTGGAAGCGATGACAAAAAAGCCAA |
| | | GGGGGCTGATGGTAAAACGAAGATCGCCACACCGCGGGGAGCAG |
| | | CCCCTCCAGGCCAGAAGGGCCAGGCCAACGCCACCAGGATTCCA |
| | | GCAAAAACCCCGCCCGCTCCAAAGACACCACCCAGCTCTGGTGA |
| | | ACCTCCAAAATCAGGGGATCGCAGCGGCTACAGCAGCCCCGGCT |
| | | CCCCAGGCACTCCCGGCAGCCGCTCCCGCACCCCGTCCCTTCCAA |
| | | CCCCACCCACCCGGGAGCCCAAGAAGGTGGCAGTGGTCCGTACTC |
| | | CACCCAAGTCGCCGTCTTCCGCCAAGAGCCGCCTGCAGACAGCCC |
| | | CCGTGCCCATGCCAGACCTGAAGAATGTCAAGTCCAAGATCGGCT |
| | | CCACTGAGAACCTGAAGCACCAGCCGGGAGGCGGGAAGGTGCAA |
| | | ATAGTCTACAAACCAGTTGACCTGAGCAAGGTGACCTCCAAGTGT |
| | | GGCTCATTAGGCAACATCCATCATAAACCAGGAGGTGGCCAGGT |
| | | GGAAGTAAAATCTGAGAAGCTTGACTTCAAGGACAGAGTCCAGT |
| | | CGAAGATTGGGTCCCTGGACAATATCACCCACGTCCCTGGCGGAG |
| | | GAAATAAAAAGATTGAAACCCACAAGCTGACCTTCCGCGAGAAC |
| | | GCCAAAGCCAAGACAGACCACGGGGCGGAGATCGTGTACAAGTC |
| | | GCCAGTGGTGTCTGGGGACACGTCTCCACGGCATCTCAGCAATGT |
| | | CTCCTCCACCGGCAGCATCGACATGGTAGACTCGCCCCAGCTCGC |
| | | CACGCTAGCTGACGAGGTGTCTGCCTCCCTGGCCAAGCAGGGTTT |
| | | GTGATCAGGCCCCTGGGGCGGTCAATAATTGTGGAGAGGAGAGA |
| | | ATGAGAGAGTGTGGAAAAAAAAAGAATAATGACCCGGCCCCCGC |
| | | CCTCTGCCCCCAGCTGCTCCTCGCAGTTCGGTTAATTGGTTAATCA |
| | | CTTAACCTGCTTTTGTCACTCGGCTTTGGCTCGGGACTTCAAAATC |
| | | AGTGATGGGAGTAAGAGCAAATTTCATCTTTCCAAATTGATGGGT |
| | | GGGCTAGTAATAAAATATTTAAAAAAAAACATTCAAAAACATGG |
| | | CCACATCCAACATTTCCTCAGGCAATTCCTTTTGATTCTTTTTTCTT |
| | | CCCCCTCCATGTAGAAGAGGGAGAAGGAGAGGCTCTGAAAGCTG |
| | | CTTCTGGGGGATTTCAAGGGACTGGGGGTGCCAACCACCTCTGGC |
| | | CCTGTTGTGGGGGTGTCACAGAGGCAGTGGCAGCAACAAAGGAT |
| | | TTGAAACTTGGTGTGTTCGTGGAGCCACAGGCAGACGATGTCAAC |
| | | CTTGTGTGAGTGTGACGGGGGTTGGGGTGGGGCGGGAGGCCACG |
| | | GGGGAGGCCGAGGCAGGGGCTGGGCAGAGGGGAGAGGAAGCAC |
| | | AAGAAGTGGGAGTGGGAGAGGAAGCCACGTGCTGGAGAGTAGAC |
| | | ATCCCCCTCCTTGCCGCTGGGAGAGCCAAGGCCTATGCCACCTGC |
| | | AGCGTCTGAGCGGCCGCCTGTCCTTGGTGGCCGGGGGTGGGGGCC |
| | | TGCTGTGGGTCAGTGTGCCACCCTCTGCAGGGCAGCCTGTGGGAG |
| | | AAGGGACAGCGGGTAAAAAGAGAAGGCAAGCTGGCAGGAGGGT |
| | | GGCACTTCGTGGATGACCTCCTTAGAAAAGACTGACCTTGATGTC |
| | | TTGAGAGCGCTGGCCTCTTCCTCCCTCCCTGCAGGGTAGGGGGCC |
| | | TGAGTTGAGGGGCTTCCCTCTGCTCCACAGAAACCCTGTTTTATTG |
| | | AGTTCTGAAGGTTGGAACTGCTGCCATGATTTTGGCCACTTTGCA |
| | | GACCTGGGACTTTAGGGCTAACCAGTTCTCTTTGTAAGGACTTGT |
| | | GCCTCTTGGGAGACGTCCACCCGTTTCCAAGCCTGGGCCACTGGC |
| | | ATCTCTGGAGTGTGTGGGGGTCTGGGAGGCAGGTCCCGAGCCCCC |
| | | TGTCCTTCCCACGGCCACTGCAGTCACCCCGTCTGCGCCGCTGTGC |
| | | TGTTGTCTGCCGTGAGAGCCCAATCACTGCCTATACCCCTCATCAC |
| | | ACGTCACAATGTCCCGAATTCCCAGCCTCACCACCCCTTCTCAGT |
| | | AATGACCCTGGTTGGTTGCAGGAGGTACCTACTCCATACTGAGGG |
| | | TGAAATTAAGGGAAGGCAAAGTCCAGGCACAAGAGTGGGACCCC |
| | | AGCCTCTCACTCTCAGTTCCACTCATCCAACTGGGACCCTCACCAC |
| | | GAATCTCATGATCTGATTCGGTTCCCTGTCTCCTCCTCCCGTCACA |
| | | GATGTGAGCCAGGGCACTGCTCAGCTGTGACCCTAGGTGTTTCTG |
| | | CCTTGTTGACATGGAGAGAGCCCTTTCCCCTGAGAAGGCCTGGCC |
| | | CCTTCCTGTGCTGAGCCCACAGCAGCAGGCTGGGTGTCTTGGTTG |
| | | TCAGTGGTGGCACCAGGATGGAAGGGCAAGGCACCCAGGGCAGG |
| | | CCCACAGTCCCGCTGTCCCCCACTTGCACCCTAGCTTGTAGCTGCC |
| | | AACCTCCCAGACAGCCCAGCCCGCTGCTCAGCTCCACATGCATAG |
| | | TATCAGCCCTCCACACCCGACAAAGGGGAACACACCCCCTTGGAA |
| | | ATGGTTCTTTTCCCCCAGTCCCAGCTGGAAGCCATGCTGTCTGTTC |
| | | TGCTGGAGCAGCTGAACATATACATAGATGTTGCCCTGCCCTCCC |
| | | CATCTGCACCCTGTTGAGTTGTAGTTGGATTTGTCTGTTTATGCTT |
| | | GGATTCACCAGAGTGACTATGATAGTGAAAAGAAAAAAAAAAAA |
| | | AAAAAAGGACGCATGTATCTTGAAATGCTTGTAAAGAGGTTTCTA |
| | | ACCCACCCTCACGAGGTGTCTCTCACCCCCACACTGGGACTCGTG |
| | | TGGCCTGTGTGGTGCCACCCTGCTGGGGCCTCCCAAGTTTTGAAA |
| | | GGCTTTCCTCAGCACCTGGGACCCAACAGAGACCAGCTTCTAGCA |
| | | GCTAAGGAGGCCGTTCAGCTGTGACGAAGGCCTGAAGCACAGGA |
| | | TTAGGACTGAAGCGATGATGTCCCCTTCCCTACTTCCCCTTGGGGC |
| | | TCCCTGTGTCAGGGCACAGACTAGGTCTTGTGGCTGGTCTGGCTT |
| | | GCGGCGCGAGGATGGTTCTCTCTGGTCATAGCCCGAAGTCTCATG |
| | | GCAGTCCCAAAGGAGGCTTACAACTCCTGCATCACAAGAAAAAG |
| | | GAAGCCACTGCCAGCTGGGGGGATCTGCAGCTCCCAGAAGCTCC |
| | | GTGAGCCTCAGCCACCCCTCAGACTGGGTTCCTCTCCAAGCTCGC |
| | | CCTCTGGAGGGGCAGCGCAGCCTCCCACCAAGGGCCCTGCGACC |
| | | ACAGCAGGGATTGGGATGAATTGCCTGTCCTGGATCTGCTCTAGA |
| | | GGCCCAAGCTGCCTGCCTGAGGAAGGATGACTTGACAAGTCAGG |
| | | AGACACTGTTCCCAAAGCCTTGACCAGAGCACCTCAGCCCGCTGA |
| | | CCTTGCACAAACTCCATCTGCTGCCATGAGAAAAGGGAAGCCGCC |
| | | TTTGCAAAACATTGCTGCCTAAAGAAACTCAGCAGCCTCAGGCCC |
| | | AATTCTGCCACTTCTGGTTTGGGTACAGTTAAAGGCAACCCTGAG |
| | | GGACTTGGCAGTAGAAATCCAGGGCCTCCCCTGGGGCTGGCAGCT |
| | | TCGTGTGCAGCTAGAGCTTTACCTGAAAGGAAGTCTCTGGGCCCA |
| | | GAACTCTCCACCAAGAGCCTCCCTGCCGTTCGCTGAGTCCCAGCA |
| | | ATTCTCCTAAGTTGAAGGGATCTGAGAAGGAGAAGGAAATGTGG |
| | | GGTAGATTTGGTGGTGGTTAGAGATATGCCCCCCTCATTACTGCC |
| | | AACAGTTTCGGCTGCATTTCTTCACGCACCTCGGTTCCTCTTCCTG |
| | | AAGTTCTTGTGCCCTGCTCTTCAGCACCATGGGCCTTCTTATACGG |
| | | AAGGCTCTGGGATCTCCCCCTTGTGGGGCAGGCTCTTGGGGCCAG |
| | | CCTAAGATCATGGTTTAGGGTGATCAGTGCTGGCAGATAAATTGA |
| | | AAAGGCACGCTGGCTTGTGATCTTAAATGAGGACAATCCCCCCAG |
| | | GGCTGGGCACTCCTCCCCTCCCCTCACTTCTCCCACCTGCAGAGCC |
| | | AGTGTCCTTGGGTGGGCTAGATAGGATATACTGTATGCCGGCTCC |
| | | TTCAAGCTGCTGACTCACTTTATCAATAGTTCCATTTAAATTGACT |
| | | TCAGTGGTGAGACTGTATCCTGTTTGCTATTGCTTGTTGTGCTATG |
| | | GGGGGAGGGGGGAGGAATGTGTAAGATAGTTAACATGGGCAAAG |
| | | GGAGATCTTGGGGTGCAGCACTTAAACTGCCTCGTAACCCTTTTC |
| | | ATGATTTCAACCACATTTGCTAGAGGGAGGGAGCAGCCACGGAG |
| | | TTAGAGGCCCTTGGGGTTTCTCTTTTCCACTGACAGGCTTTCCCAG |
| | | GCAGCTGGCTAGTTCATTCCCTCCCCAGCCAGGTGCAGGCGTAGG |
| | | AATATGGACATCTGGTTGCTTTGGCCTGCTGCCCTCTTTCAGGGGT |
| | | CCTAAGCCCACAATCATGCCTCCCTAAGACCTTGGCATCCTTCCCT |
| | | CTAAGCCGTTGGCACCTCTGTGCCACCTCTCACACTGGCTCCAGA |
| | | CACACAGCCTGTGCTTTTGGAGCTGAGATCACTCGCTTCACCCTCC |
| | | TCATCTTTGTTCTCCAAGTAAAGCCACGAGGTCGGGGCGAGGGCA |
| | | GAGGTGATCACCTGCGTGTCCCATCTACAGACCTGCAGCTTCATA |
| | | AAACTTCTGATTTCTCTTCAGCTTTGAAAAGGGTTACCCTGGGCAC |
| | | TGGCCTAGAGCCTCACCTCCTAATAGACTTAGCCCCATGAGTTTG |
| | | CCATGTTGAGCAGGACTATTTCTGGCACTTGCAAGTCCCATGATTT |
| | | CTTCGGTAATTCTGAGGGTGGGGGGAGGGACATGAAATCATCTTA |
| | | GCTTAGCTTTCTGTCTGTGAATGTCTATATAGTGTATTGTGTGTTT |
| | | TAACAAATGATTTACACTGACTGTTGCTGTAAAAGTGAATTTGGA |
| | | AATAAAGTTATTACTCTGATTAAA |
| 47 | 11 | GCAGTCACCGCCACCCACCAGCTCCGGCACCAACAGCAGCGCCG |
| | | CTGCCACCGCCCACCTTCTGCCGCCGCCACCACAGCCACCTTCTCC |
| | | TCCTCCGCTGTCCTCTCCCGTCCTCGCCTCTGTCGACTATCAGGTG |
| | | AACTTTGAACCAGGATGGCTGAGCCCCGCCAGGAGTTCGAAGTG |
| | | ATGGAAGATCACGCTGGGACGTACGGGTTGGGGGACAGGAAAGA |
| | | TCAGGGGGGCTACACCATGCACCAAGACCAAGAGGGTGACACGG |
| | | ACGCTGGCCTGAAAGAATCTCCCCTGCAGACCCCCACTGAGGACG |
| | | GATCTGAGGAACCGGGCTCTGAAACCTCTGATGCTAAGAGCACTC |
| | | CAACAGCGGAAGCTGAAGAAGCAGGCATTGGAGACACCCCCAGC |
| | | CTGGAAGACGAAGCTGCTGGTCACGTGACCCAAGCTCGCATGGTC |
| | | AGTAAAAGCAAAGACGGGACTGGAAGCGATGACAAAAAAGCCA |
| | | AGGGGGCTGATGGTAAAACGAAGATCGCCACACCGCGGGGAGCA |
| | | GCCCCTCCAGGCCAGAAGGGCCAGGCCAACGCCACCAGGATTCC |
| | | AGCAAAAACCCCGCCCGCTCCAAAGACACCACCCAGCTCTGGTG |
| | | AACCTCCAAAATCAGGGGATCGCAGCGGCTACAGCAGCCCCGGC |
| | | TCCCCAGGCACTCCCGGCAGCCGCTCCCGCACCCCGTCCCTTCCA |
| | | ACCCCACCCACCCGGGAGCCCAAGAAGGTGGCAGTGGTCCGTAC |
| | | TCCACCCAAGTCGCCGTCTTCCGCCAAGAGCCGCCTGCAGACAGC |
| | | CCCCGTGCCCATGCCAGACCTGAAGAATGTCAAGTCCAAGATCGG |
| | | CTCCACTGAGAACCTGAAGCACCAGCCGGGAGGCGGGAAGGTGC |
| | | AAATAGTCTACAAACCAGTTGACCTGAGCAAGGTTGGAACTGCTG |
| | | CCATGATTTTGGCCACTTTGCAGACCTGGGACTTTAGGGCTAACC |
| | | AGTTCTCTTTGTAAGGACTTGTGCCTCTTGGGAGACGTCCACCCGT |
| | | TTCCAAGCCTGGGCCACTGGCATCTCTGGAGTGTGTGGGGGTCTG |
| | | GGAGGCAGGTCCCGAGCCCCCTGTCCTTCCCACGGCCACTGCAGT |
| | | CACCCCGTCTGCGCCGCTGTGCTGTTGTCTGCCGTGAGAGCCCAA |
| | | TCACTGCCTATACCCCTCATCACACGTCACAATGTCCCGAATTCCC |
| | | AGCCTCACCACCCCTTCTCAGTAATGACCCTGGTTGGTTGCAGGA |
| | | GGTACCTACTCCATACTGAGGGTGAAATTAAGGGAAGGCAAAGT |
| | | CCAGGCACAAGAGTGGGACCCCAGCCTCTCACTCTCAGTTCCACT |
| | | CATCCAACTGGGACCCTCACCACGAATCTCATGATCTGATTCGGT |
| | | TCCCTGTCTCCTCCTCCCGTCACAGATGTGAGCCAGGGCACTGCTC |
| | | AGCTGTGACCCTAGGTGTTTCTGCCTTGTTGACATGGAGAGAGCC |
| | | CTTTCCCCTGAGAAGGCCTGGCCCCTTCCTGTGCTGAGCCCACAG |
| | | CAGCAGGCTGGGTGTCTTGGTTGTCAGTGGTGGCACCAGGATGGA |
| | | AGGGCAAGGCACCCAGGGCAGGCCCACAGTCCCGCTGTCCCCCA |
| | | CTTGCACCCTAGCTTGTAGCTGCCAACCTCCCAGACAGCCCAGCC |
| | | CGCTGCTCAGCTCCACATGCATAGTATCAGCCCTCCACACCCGAC |
| | | AAAGGGGAACACACCCCCTTGGAAATGGTTCTTTTCCCCCAGTCC |
| | | CAGCTGGAAGCCATGCTGTCTGTTCTGCTGGAGCAGCTGAACATA |
| | | TACATAGATGTTGCCCTGCCCTCCCCATCTGCACCCTGTTGAGTTG |
| | | TAGTTGGATTTGTCTGTTTATGCTTGGATTCACCAGAGTGACTATG |
| | | ATAGTGAAAAGAAAAAAAAAAAAAAAAAAGGACGCATGTATCTT |
| | | GAAATGCTTGTAAAGAGGTTTCTAACCCACCCTCACGAGGTGTCT |
| | | CTCACCCCCACACTGGGACTCGTGTGGCCTGTGTGGTGCCACCCT |
| | | GCTGGGGCCTCCCAAGTTTTGAAAGGCTTTCCTCAGCACCTGGGA |
| | | CCCAACAGAGACCAGCTTCTAGCAGCTAAGGAGGCCGTTCAGCTG |
| | | TGACGAAGGCCTGAAGCACAGGATTAGGACTGAAGCGATGATGT |
| | | CCCCTTCCCTACTTCCCCTTGGGGCTCCCTGTGTCAGGGCACAGAC |
| | | TAGGTCTTGTGGCTGGTCTGGCTTGCGGCGCGAGGATGGTTCTCT |
| | | CTGGTCATAGCCCGAAGTCTCATGGCAGTCCCAAAGGAGGCTTAC |
| | | AACTCCTGCATCACAAGAAAAAGGAAGCCACTGCCAGCTGGGGG |
| | | GATCTGCAGCTCCCAGAAGCTCCGTGAGCCTCAGCCACCCCTCAG |
| | | ACTGGGTTCCTCTCCAAGCTCGCCCTCTGGAGGGGCAGCGCAGCC |
| | | TCCCACCAAGGGCCCTGCGACCACAGCAGGGATTGGGATGAATT |
| | | GCCTGTCCTGGATCTGCTCTAGAGGCCCAAGCTGCCTGCCTGAGG |
| | | AAGGATGACTTGACAAGTCAGGAGACACTGTTCCCAAAGCCTTGA |
| | | CCAGAGCACCTCAGCCCGCTGACCTTGCACAAACTCCATCTGCTG |
| | | CCATGAGAAAAGGGAAGCCGCCTTTGCAAAACATTGCTGCCTAA |
| | | AGAAACTCAGCAGCCTCAGGCCCAATTCTGCCACTTCTGGTTTGG |
| | | GTACAGTTAAAGGCAACCCTGAGGGACTTGGCAGTAGAAATCCA |
| | | GGGCCTCCCCTGGGGCTGGCAGCTTCGTGTGCAGCTAGAGCTTTA |
| | | CCTGAAAGGAAGTCTCTGGGCCCAGAACTCTCCACCAAGAGCCTC |
| | | CCTGCCGTTCGCTGAGTCCCAGCAATTCTCCTAAGTTGAAGGGAT |
| | | CTGAGAAGGAGAAGGAAATGTGGGGTAGATTTGGTGGTGGTTAG |
| | | AGATATGCCCCCCTCATTACTGCCAACAGTTTCGGCTGCATTTCTT |
| | | CACGCACCTCGGTTCCTCTTCCTGAAGTTCTTGTGCCCTGCTCTTC |
| | | AGCACCATGGGCCTTCTTATACGGAAGGCTCTGGGATCTCCCCCT |
| | | TGTGGGGCAGGCTCTTGGGGCCAGCCTAAGATCATGGTTTAGGGT |
| | | GATCAGTGCTGGCAGATAAATTGAAAAGGCACGCTGGCTTGTGAT |
| | | CTTAAATGAGGACAATCCCCCCAGGGCTGGGCACTCCTCCCCTCC |
| | | CCTCACTTCTCCCACCTGCAGAGCCAGTGTCCTTGGGTGGGCTAG |
| | | ATAGGATATACTGTATGCCGGCTCCTTCAAGCTGCTGACTCACTTT |
| | | ATCAATAGTTCCATTTAAATTGACTTCAGTGGTGAGACTGTATCCT |
| | | GTTTGCTATTGCTTGTTGTGCTATGGGGGGAGGGGGGAGGAATGT |
| | | GTAAGATAGTTAACATGGGCAAAGGGAGATCTTGGGGTGCAGCA |
| | | CTTAAACTGCCTCGTAACCCTTTTCATGATTTCAACCACATTTGCT |
| | | AGAGGGAGGGAGCAGCCACGGAGTTAGAGGCCCTTGGGGTTTCT |
| | | CTTTTCCACTGACAGGCTTTCCCAGGCAGCTGGCTAGTTCATTCCC |
| | | TCCCCAGCCAGGTGCAGGCGTAGGAATATGGACATCTGGTTGCTT |
| | | TGGCCTGCTGCCCTCTTTCAGGGGTCCTAAGCCCACAATCATGCCT |
| | | CCCTAAGACCTTGGCATCCTTCCCTCTAAGCCGTTGGCACCTCTGT |
| | | GCCACCTCTCACACTGGCTCCAGACACACAGCCTGTGCTTTTGGA |
| | | GCTGAGATCACTCGCTTCACCCTCCTCATCTTTGTTCTCCAAGTAA |
| | | AGCCACGAGGTCGGGGCGAGGGCAGAGGTGATCACCTGCGTGTC |
| | | CCATCTACAGACCTGCAGCTTCATAAAACTTCTGATTTCTCTTCAG |
| | | CTTTGAAAAGGGTTACCCTGGGCACTGGCCTAGAGCCTCACCTCC |
| | | TAATAGACTTAGCCCCATGAGTTTGCCATGTTGAGCAGGACTATT |
| | | TCTGGCACTTGCAAGTCCCATGATTTCTTCGGTAATTCTGAGGGTG |
| | | GGGGGAGGGACATGAAATCATCTTAGCTTAGCTTTCTGTCTGTGA |
| | | ATGTCTATATAGTGTATTGTGTGTTTTAACAAATGATTTACACTGA |
| | | CTGTTGCTGTAAAAGTGAATTTGGAAATAAAGTTATTACTCTGAT |
| | | TAAA |
| 48 | 12 | GCAGTCACCGCCACCCACCAGCTCCGGCACCAACAGCAGCGCCG |
| | | CTGCCACCGCCCACCTTCTGCCGCCGCCACCACAGCCACCTTCTCC |
| | | TCCTCCGCTGTCCTCTCCCGTCCTCGCCTCTGTCGACTATCAGACA |
| | | GGCTCTGCTGATGCTGTCCCTCTCCTGTTCAGTCGTGCCCTCACCG |
| | | TTAAAGAGAAAGAGCAAACTGCTGGGCAGCAGCATTGATTTTTTT |
| | | AATGAAGTGGAAAGAGAGCTGGGAATAACAAGTCGGGCCCACCT |
| | | CACCTGCCTCACCTGGTGAACTTTGAACCAGGATGGCTGAGCCCC |
| | | GCCAGGAGTTCGAAGTGATGGAAGATCACGCTGGGACGTACGGG |
| | | TTGGGGGACAGGAAAGATCAGGGGGGCTACACCATGCACCAAGA |
| | | CCAAGAGGGTGACACGGACGCTGGCCTGAAAGCTGAAGAAGCAG |
| | | GCATTGGAGACACCCCCAGCCTGGAAGACGAAGCTGCTGGTCAC |
| | | GTGACCCAAGCTCGCATGGTCAGTAAAAGCAAAGACGGGACTGG |
| | | AAGCGATGACAAAAAAGCCAAGGGGGCTGATGGTAAAACGAAG |
| | | ATCGCCACACCGCGGGGAGCAGCCCCTCCAGGCCAGAAGGGCCA |
| | | GGCCAACGCCACCAGGATTCCAGCAAAAACCCCGCCCGCTCCAA |
| | | AGACACCACCCAGCTCTGGTGAACCTCCAAAATCAGGGGATCGC |
| | | AGCGGCTACAGCAGCCCCGGCTCCCCAGGCACTCCCGGCAGCCGC |
| | | TCCCGCACCCCGTCCCTTCCAACCCCACCCACCCGGGAGCCCAAG |
| | | AAGGTGGCAGTGGTCCGTACTCCACCCAAGTCGCCGTCTTCCGCC |
| | | AAGAGCCGCCTGCAGACAGCCCCCGTGCCCATGCCAGACCTGAA |
| | | GAATGTCAAGTCCAAGATCGGCTCCACTGAGAACCTGAAGCACC |
| | | AGCCGGGAGGCGGGAAGGTGCAAATAGTCTACAAACCAGTTGAC |
| | | CTGAGCAAGGTGACCTCCAAGTGTGGCTCATTAGGCAACATCCAT |
| | | CATAAACCAGGAGGTGGCCAGGTGGAAGTAAAATCTGAGAAGCT |
| | | TGACTTCAAGGACAGAGTCCAGTCGAAGATTGGGTCCCTGGACAA |
| | | TATCACCCACGTCCCTGGCGGAGGAAATAAAAAGATTGAAACCC |
| | | ACAAGCTGACCTTCCGCGAGAACGCCAAAGCCAAGACAGACCAC |
| | | GGGGCGGAGATCGTGTACAAGTCGCCAGTGGTGTCTGGGGACAC |
| | | GTCTCCACGGCATCTCAGCAATGTCTCCTCCACCGGCAGCATCGA |
| | | CATGGTAGACTCGCCCCAGCTCGCCACGCTAGCTGACGAGGTGTC |
| | | TGCCTCCCTGGCCAAGCAGGGTTTGTGATCAGGCCCCTGGGGCGG |
| | | TCAATAATTGTGGAGAGGAGAGAATGAGAGAGTGTGGAAAAAAA |
| | | AAGAATAATGACCCGGCCCCCGCCCTCTGCCCCCAGCTGCTCCTC |
| | | GCAGTTCGGTTAATTGGTTAATCACTTAACCTGCTTTTGTCACTCG |
| | | GCTTTGGCTCGGGACTTCAAAATCAGTGATGGGAGTAAGAGCAA |
| | | ATTTCATCTTTCCAAATTGATGGGTGGGCTAGTAATAAAATATTTA |
| | | AAAAAAAACATTCAAAAACATGGCCACATCCAACATTTCCTCAGG |
| | | CAATTCCTTTTGATTCTTTTTTCTTCCCCCTCCATGTAGAAGAGGG |
| | | AGAAGGAGAGGCTCTGAAAGCTGCTTCTGGGGGATTTCAAGGGA |
| | | CTGGGGGTGCCAACCACCTCTGGCCCTGTTGTGGGGGTGTCACAG |
| | | AGGCAGTGGCAGCAACAAAGGATTTGAAACTTGGTGTGTTCGTGG |
| | | AGCCACAGGCAGACGATGTCAACCTTGTGTGAGTGTGACGGGGG |
| | | TTGGGGTGGGGCGGGAGGCCACGGGGGAGGCCGAGGCAGGGGCT |
| | | GGGCAGAGGGGAGAGGAAGCACAAGAAGTGGGAGTGGGAGAGG |
| | | AAGCCACGTGCTGGAGAGTAGACATCCCCCTCCTTGCCGCTGGGA |
| | | GAGCCAAGGCCTATGCCACCTGCAGCGTCTGAGCGGCCGCCTGTC |
| | | CTTGGTGGCCGGGGGTGGGGGCCTGCTGTGGGTCAGTGTGCCACC |
| | | CTCTGCAGGGCAGCCTGTGGGAGAAGGGACAGCGGGTAAAAAGA |
| | | GAAGGCAAGCTGGCAGGAGGGTGGCACTTCGTGGATGACCTCCTT |
| | | AGAAAAGACTGACCTTGATGTCTTGAGAGCGCTGGCCTCTTCCTC |
| | | CCTCCCTGCAGGGTAGGGGGCCTGAGTTGAGGGGCTTCCCTCTGC |
| | | TCCACAGAAACCCTGTTTTATTGAGTTCTGAAGGTTGGAACTGCT |
| | | GCCATGATTTTGGCCACTTTGCAGACCTGGGACTTTAGGGCTAAC |
| | | CAGTTCTCTTTGTAAGGACTTGTGCCTCTTGGGAGACGTCCACCCG |
| | | TTTCCAAGCCTGGGCCACTGGCATCTCTGGAGTGTGTGGGGGTCT |
| | | GGGAGGCAGGTCCCGAGCCCCCTGTCCTTCCCACGGCCACTGCAG |
| | | TCACCCCGTCTGCGCCGCTGTGCTGTTGTCTGCCGTGAGAGCCCA |
| | | ATCACTGCCTATACCCCTCATCACACGTCACAATGTCCCGAATTCC |
| | | CAGCCTCACCACCCCTTCTCAGTAATGACCCTGGTTGGTTGCAGG |
| | | AGGTACCTACTCCATACTGAGGGTGAAATTAAGGGAAGGCAAAG |
| | | TCCAGGCACAAGAGTGGGACCCCAGCCTCTCACTCTCAGTTCCAC |
| | | TCATCCAACTGGGACCCTCACCACGAATCTCATGATCTGATTCGG |
| | | TTCCCTGTCTCCTCCTCCCGTCACAGATGTGAGCCAGGGCACTGCT |
| | | CAGCTGTGACCCTAGGTGTTTCTGCCTTGTTGACATGGAGAGAGC |
| | | CCTTTCCCCTGAGAAGGCCTGGCCCCTTCCTGTGCTGAGCCCACA |
| | | GCAGCAGGCTGGGTGTCTTGGTTGTCAGTGGTGGCACCAGGATGG |
| | | AAGGGCAAGGCACCCAGGGCAGGCCCACAGTCCCGCTGTCCCCC |
| | | ACTTGCACCCTAGCTTGTAGCTGCCAACCTCCCAGACAGCCCAGC |
| | | CCGCTGCTCAGCTCCACATGCATAGTATCAGCCCTCCACACCCGA |
| | | CAAAGGGGAACACACCCCCTTGGAAATGGTTCTTTTCCCCCAGTC |
| | | CCAGCTGGAAGCCATGCTGTCTGTTCTGCTGGAGCAGCTGAACAT |
| | | ATACATAGATGTTGCCCTGCCCTCCCCATCTGCACCCTGTTGAGTT |
| | | GTAGTTGGATTTGTCTGTTTATGCTTGGATTCACCAGAGTGACTAT |
| | | GATAGTGAAAAGAAAAAAAAAAAAAAAAAAGGACGCATGTATCT |
| | | TGAAATGCTTGTAAAGAGGTTTCTAACCCACCCTCACGAGGTGTC |
| | | TCTCACCCCCACACTGGGACTCGTGTGGCCTGTGTGGTGCCACCC |
| | | TGCTGGGGCCTCCCAAGTTTTGAAAGGCTTTCCTCAGCACCTGGG |
| | | ACCCAACAGAGACCAGCTTCTAGCAGCTAAGGAGGCCGTTCAGCT |
| | | GTGACGAAGGCCTGAAGCACAGGATTAGGACTGAAGCGATGATG |
| | | TCCCCTTCCCTACTTCCCCTTGGGGCTCCCTGTGTCAGGGCACAGA |
| | | CTAGGTCTTGTGGCTGGTCTGGCTTGCGGCGCGAGGATGGTTCTC |
| | | TCTGGTCATAGCCCGAAGTCTCATGGCAGTCCCAAAGGAGGCTTA |
| | | CAACTCCTGCATCACAAGAAAAAGGAAGCCACTGCCAGCTGGGG |
| | | GGATCTGCAGCTCCCAGAAGCTCCGTGAGCCTCAGCCACCCCTCA |
| | | GACTGGGTTCCTCTCCAAGCTCGCCCTCTGGAGGGGCAGCGCAGC |
| | | CTCCCACCAAGGGCCCTGCGACCACAGCAGGGATTGGGATGAATT |
| | | GCCTGTCCTGGATCTGCTCTAGAGGCCCAAGCTGCCTGCCTGAGG |
| | | AAGGATGACTTGACAAGTCAGGAGACACTGTTCCCAAAGCCTTGA |
| | | CCAGAGCACCTCAGCCCGCTGACCTTGCACAAACTCCATCTGCTG |
| | | CCATGAGAAAAGGGAAGCCGCCTTTGCAAAACATTGCTGCCTAA |
| | | AGAAACTCAGCAGCCTCAGGCCCAATTCTGCCACTTCTGGTTTGG |
| | | GTACAGTTAAAGGCAACCCTGAGGGACTTGGCAGTAGAAATCCA |
| | | GGGCCTCCCCTGGGGCTGGCAGCTTCGTGTGCAGCTAGAGCTTTA |
| | | CCTGAAAGGAAGTCTCTGGGCCCAGAACTCTCCACCAAGAGCCTC |
| | | CCTGCCGTTCGCTGAGTCCCAGCAATTCTCCTAAGTTGAAGGGAT |
| | | CTGAGAAGGAGAAGGAAATGTGGGGTAGATTTGGTGGTGGTTAG |
| | | AGATATGCCCCCCTCATTACTGCCAACAGTTTCGGCTGCATTTCTT |
| | | CACGCACCTCGGTTCCTCTTCCTGAAGTTCTTGTGCCCTGCTCTTC |
| | | AGCACCATGGGCCTTCTTATACGGAAGGCTCTGGGATCTCCCCCT |
| | | TGTGGGGCAGGCTCTTGGGGCCAGCCTAAGATCATGGTTTAGGGT |
| | | GATCAGTGCTGGCAGATAAATTGAAAAGGCACGCTGGCTTGTGAT |
| | | CTTAAATGAGGACAATCCCCCCAGGGCTGGGCACTCCTCCCCTCC |
| | | CCTCACTTCTCCCACCTGCAGAGCCAGTGTCCTTGGGTGGGCTAG |
| | | ATAGGATATACTGTATGCCGGCTCCTTCAAGCTGCTGACTCACTTT |
| | | ATCAATAGTTCCATTTAAATTGACTTCAGTGGTGAGACTGTATCCT |
| | | GTTTGCTATTGCTTGTTGTGCTATGGGGGGAGGGGGGAGGAATGT |
| | | GTAAGATAGTTAACATGGGCAAAGGGAGATCTTGGGGTGCAGCA |
| | | CTTAAACTGCCTCGTAACCCTTTTCATGATTTCAACCACATTTGCT |
| | | AGAGGGAGGGAGCAGCCACGGAGTTAGAGGCCCTTGGGGTTTCT |
| | | CTTTTCCACTGACAGGCTTTCCCAGGCAGCTGGCTAGTTCATTCCC |
| | | TCCCCAGCCAGGTGCAGGCGTAGGAATATGGACATCTGGTTGCTT |
| | | TGGCCTGCTGCCCTCTTTCAGGGGTCCTAAGCCCACAATCATGCCT |
| | | CCCTAAGACCTTGGCATCCTTCCCTCTAAGCCGTTGGCACCTCTGT |
| | | GCCACCTCTCACACTGGCTCCAGACACACAGCCTGTGCTTTTGGA |
| | | GCTGAGATCACTCGCTTCACCCTCCTCATCTTTGTTCTCCAAGTAA |
| | | AGCCACGAGGTCGGGGCGAGGGCAGAGGTGATCACCTGCGTGTC |
| | | CCATCTACAGACCTGCAGCTTCATAAAACTTCTGATTTCTCTTCAG |
| | | CTTTGAAAAGGGTTACCCTGGGCACTGGCCTAGAGCCTCACCTCC |
| | | TAATAGACTTAGCCCCATGAGTTTGCCATGTTGAGCAGGACTATT |
| | | TCTGGCACTTGCAAGTCCCATGATTTCTTCGGTAATTCTGAGGGTG |
| | | GGGGGAGGGACATGAAATCATCTTAGCTTAGCTTTCTGTCTGTGA |
| | | ATGTCTATATAGTGTATTGTGTGTTTTAACAAATGATTTACACTGA |
| | | CTGTTGCTGTAAAAGTGAATTTGGAAATAAAGTTATTACTCTGAT |
| | | TAAA |

### PTEN-Induced Kinase 1 (PINK1)

PINK1 encodes a mitochondrial serine/threonine-protein kinase. It is ubiquitously expressed, with the highest expression in the heart, muscles, and testes. It functions in the protection of mitochondrial function during stress, the mitochondrial quality control, mitochondrial fission, and mitochondrial mobility. In the nervous system, PINK1 is also processed and released by mitochondria to regulate neuronal differentiation. Mutations in this gene has been shown to lead to the build-up of Lewy bodies and cause one form of autosomal recessive Parkinson's Disease.

In an embodiment, a specific nucleotide residue can be targeted utilizing compositions and methods provided herein. Exemplary complete PINK1 mRNA sequences are shown in Table 8. In some cases, a target nucleotide residue can be at any position of the 2,657 nucleotide residues of a sequence that may be targeted utilizing the compositions and method provided herein. In some cases, a target nucleotide residue may be located among nucleotide residues 1-100, 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, 1901-2000, 2001-2100, 2101-2200, 2201-2300, 2301-2400, 2401-2500, 2501-2600, 2601-2657, or any combination thereof of the PINK1 mRNA.

In some embodiments, engineered polynucleotide facilitates editing of the translation initiation site (TIS) of the PINK1 mRNA (e.g., editing the A of the ATG codon). In some embodiments, the editing of the TIS results in a knockdown of expression the PINK1 polypeptide from the edited PINK1 mRNA.

TABLE 8
Human PINK1 mRNA Isoform Sequences. Sequences obtained from NCBI PINK1
gene ID: 65018; Assembly GRCh38.p13 (GCF_000001405.39); NC_000001.11
(20633458..20651511)

| SEQ ID | | |
|---|---|---|
| NO: | Isoform | mRNA sequence |
| 49 | 1 | AGAGGCACCGCCCCAAGTTTGTTGTGACCGGCGGGGGACGCCGG |
| | | TGGTGGCGGCAGCGGCGGCTGCGGGGGCACCGGGCCGCGGCGC |
| | | CACCATGGCGGTGCGACAGGCGCTGGGCCGCGGCCTGCAGCTGG |
| | | GTCGAGCGCTGCTGCTGCGCTTCACGGGCAAGCCCGGCCGGGCC |
| | | TACGGCTTGGGGCGGCCGGGCCCGGCGGCGGGCTGTGTCCGCGG |
| | | GGAGCGTCCAGGCTGGGCCGCAGGACCGGGCGCGGAGCCTCGC |
| | | AGGGTCGGGCTCGGGCTCCCTAACCGTCTCCGCTTCTTCCGCCAG |
| | | TCGGTGGCCGGGCTGGCGGCGCGGTTGCAGCGGCAGTTCGTGGT |
| | | GCGGGCCTGGGGCTGCGCGGGCCCTTGCGGCCGGGCAGTCTTTC |
| | | TGGCCTTCGGGCTAGGGCTGGGCCTCATCGAGGAAAAACAGGCG |
| | | GAGAGCCGGCGGGCGGTCTCGGCCTGTCAGGAGATCCAGGCAAT |
| | | TTTTACCCAGAAAAGCAAGCCGGGGCCTGACCCGTTGGACACGA |
| | | GACGCTTGCAGGGCTTTCGGCTGGAGGAGTATCTGATAGGGCAG |
| | | TCCATTGGTAAGGGCTGCAGTGCTGCTGTGTATGAAGCCACCAT |
| | | GCCTACATTGCCCCAGAACCTGGAGGTGACAAAGAGCACCGGGT |
| | | TGCTTCCAGGGAGAGGCCCAGGTACCAGTGCACCAGGAGAAGG |
| | | GCAGGAGCGAGCTCCGGGGGCCCCTGCCTTCCCCTTGGCCATCA |
| | | AGATGATGTGGAACATCTCGGCAGGTTCCTCCAGCGAAGCCATC |
| | | TTGAACACAATGAGCCAGGAGCTGGTCCCAGCGAGCCGAGTGGC |
| | | CTTGGCTGGGGAGTATGGAGCAGTCACTTACAGAAAATCCAAGA |
| | | GAGGTCCCAAGCAACTAGCCCCTCACCCCAACATCATCCGGGTT |
| | | CTCCGCGCCTTCACCTCTTCCGTGCCGCTGCTGCCAGGGGCCCTG |
| | | GTCGACTACCCTGATGTGCTGCCCTCACGCCTCCACCCTGAAGGC |
| | | CTGGGCCATGGCCGGACGCTGTTCCTCGTTATGAAGAACTATCCC |
| | | TGTACCCTGCGCCAGTACCTTTGTGTGAACACACCCAGCCCCCGC |
| | | CTCGCCGCCATGATGCTGCTGCAGCTGCTGGAAGGCGTGGACCA |
| | | TCTGGTTCAACAGGGCATCGCGCACAGAGACCTGAAATCCGACA |
| | | ACATCCTTGTGGAGCTGGACCCAGACGGCTGCCCCTGGCTGGTG |
| | | ATCGCAGATTTTGGCTGCTGCCTGGCTGATGAGAGCATCGGCCTG |
| | | CAGTTGCCCTTCAGCAGCTGGTACGTGGATCGGGGCGGAAACGG |
| | | CTGTCTGATGGCCCCAGAGGTGTCCACGGCCCGTCCTGGCCCCA |
| | | GGGCAGTGATTGACTACAGCAAGGCTGATGCCTGGGCAGTGGGA |
| | | GCCATCGCCTATGAAATCTTCGGGCTTGTCAATCCCTTCTACGGC |
| | | CAGGGCAAGGCCCACCTTGAAAGCCGCAGCTACCAAGAGGCTCA |
| | | GCTACCTGCACTGCCCGAGTCAGTGCCTCCAGACGTGAGACAGT |
| | | TGGTGAGGGCACTGCTCCAGCGAGAGGCCAGCAAGAGACCATCT |
| | | GCCCGAGTAGCCGCAAATGTGCTTCATCTAAGCCTCTGGGGTGA |
| | | ACATATTCTAGCCCTGAAGAATCTGAAGTTAGACAAGATGGTTG |
| | | GCTGGCTCCTCCAACAATCGGCCGCCACTTTGTTGGCCAACAGGC |
| | | TCACAGAGAAGTGTTGTGTGGAAACAAAAATGAAGATGCTCTTT |
| | | CTGGCTAACCTGGAGTGTGAAACGCTCTGCCAGGCAGCCCTCCT |
| | | CCTCTGCTCATGGAGGGCAGCCCTGTGATGTCCCTGCATGGAGCT |
| | | GGTGAATTACTAAAAGAACATGGCATCCTCTGTGTCGTGATGGT |
| | | CTGTGAATGGTGAGGGTGGGAGTCAGGAGACAAGACAGCGCAG |
| | | AGAGGGCTGGTTAGCCGGAAAAGGCCTCGGGCTTGGCAAATGGA |
| | | AGAACTTGAGTGAGAGTTCAGTCTGCAGTCCTCTGCTCACAGAC |
| | | ATCTGAAAAGTGAATGGCCAAGCTGGTCTAGTAGATGAGGCTGG |
| | | ACTGAGGAGGGGTAGGCCTGCATCCACAGAGAGGATCCAGGCC |
| | | AAGGCACTGGCTGTCAGTGGCAGAGTTTGGCTGTGACCTTTGCCC |
| | | CTAACACGAGGAACTCGTTTGAAGGGGGCAGCGTAGCATGTCTG |
| | | ATTTGCCACCTGGATGAAGGCAGACATCAACATGGGTCAGCACG |
| | | TTCAGTTACGGGAGTGGGAAATTACATGAGGCCTGGGCCTCTGC |
| | | GTTCCCAAGCTGTGCGTTCTGGACCAGCTACTGAATTATTAATCT |
| | | CACTTAGCGAAAGTGACGGATGAGCAGTAAGTAAGTAAGTGTGG |
| | | GGATTTAAACTTGAGGGTTTCCCTCCTGACTAGCCTCTCTTACAG |
| | | GAATTGTGAAATATTAAATGCAAATTTACAACTGCAGATGACGT |
| | | ATGTGCCTTGAACTGAATATTTGGCTTTAAGAATGATTCTTATAC |
| | | TCTGAAGGTGAGAATATTTTGTGGGCAGGTATCAACATTGGGGA |
| | | AGAGATTTCATGTCTAACTAACTAACTTTATACATGATTTTTAGG |
| | | AAGCTATTGCCTAAATCAGCGTCAACATGCAGTAAAGGTTGTCTT |
| | | CAACTGA |

### Glucosylceramidase Beta (GBA)

GBA, also called β-Glucocerebrosidase, encodes a lysosomal membrane associated enzyme involved in the glycolipid metabolism. GBA cleaves the beta-glucosidic linkage of glucocerebroside during membrane biogenesis. The GBA protein contains three domains (I, II, and II). Domain I is necessary for the catalytic activity. Domain III binds to the substrate and contains the active site. Mutations in Deficiency caused by the mutations in the GBA gene have been implicated in Parkinson's Disease and Gaucher's Disease. It has been hypothesized that gain-of-function mutations in GBA can promote the aggregation of alpha-synuclein while loss-of-function mutations can affect the processing and clearance of alpha-synuclein.

In an embodiment, a specific nucleotide residue can be targeted utilizing compositions and methods provided herein. Exemplary complete GBA mRNA sequences are shown in Table 9. In some cases, a target nucleotide residue can be at any position of the 2,344 nucleotide residues of a sequence that may be targeted utilizing the compositions and method provided herein. In some cases, a target nucleotide residue may be located among nucleotide residues 1-100, 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, 1901-2000, 2001-2100, 2101-2200, 2201-2300, 2301-2344, or any combination thereof of the GBA mRNA. In some embodiments, engineered polynucleotide facilitates editing of the translation initiation site (TIS) of the GBA mRNA (e.g., editing the A of the ATG codon). In some embodiments, the editing of the TIS results in a knockdown of expression the GBA polypeptide from the edited GBA mRNA.

TABLE 9
Human GBA mRNA Isoform Sequences. Sequences obtained from NCBI GBA
gene ID: 2629; Assembly GRCh38.p13 (GCF_000001405.39); NC_000001.11
(155234452..155244627, complement)

| SEQ | | |
|---|---|---|
| ID NO: | Isoform | mRNA sequence |
| 50 | 1 | GGAATTACTTGCAGGGCTAACCTAGTGCCTATAGCTAAGGCAG |
| | | GTACCTGCATCCTTGTTTTTGTTTAGTGGATCCTCTATCCTTCAG |
| | | AGACTCTGGAACCCCTGTGGTCTTCTCTTCATCTAATGACCCTG |
| | | AGGGGATGGAGTTTTCAAGTCCTTCCAGAGAGGAATGTCCCAA |
| | | GCCTTTGAGTAGGGTAAGCATCATGGCTGGCAGCCTCACAGGA |
| | | TTGCTTCTACTTCAGGCAGTGTCGTGGGCATCAGGTGCCCGCCC |
| | | CTGCATCCCTAAAAGCTTCGGCTACAGCTCGGTGGTGTGTGTCT |
| | | GCAATGCCACATACTGTGACTCCTTTGACCCCCCGACCTTTCCT |
| | | GCCCTTGGTACCTTCAGCCGCTATGAGAGTACACGCAGTGGGC |
| | | GACGGATGGAGCTGAGTATGGGGCCCATCCAGGCTAATCACAC |
| | | GGGCACAGGCCTGCTACTGACCCTGCAGCCAGAACAGAAGTTC |
| | | CAGAAAGTGAAGGGATTTGGAGGGGCCATGACAGATGCTGCT |
| | | GCTCTCAACATCCTTGCCCTGTCACCCCCTGCCCAAAATTTGCT |
| | | ACTTAAATCGTACTTCTCTGAAGAAGGAATCGGATATAACATC |
| | | ATCCGGGTACCCATGGCCAGCTGTGACTTCTCCATCCGCACCTA |
| | | CACCTATGCAGACACCCCTGATGATTTCCAGTTGCACAACTTCA |
| | | GCCTCCCAGAGGAAGATACCAAGCTCAAGATACCCCTGATTCA |
| | | CCGAGCCCTGCAGTTGGCCCAGCGTCCCGTTTCACTCCTTGCCA |
| | | GCCCCTGGACATCACCCACTTGGCTCAAGACCAATGGAGCGGT |
| | | GAATGGGAAGGGGTCACTCAAGGGACAGCCCGGAGACATCTA |
| | | CCACCAGACCTGGGCCAGATACTTTGTGAAGTTCCTGGATGCC |
| | | TATGCTGAGCACAAGTTACAGTTCTGGGCAGTGACAGCTGAAA |
| | | ATGAGCCTTCTGCTGGGCTGTTGAGTGGATACCCCTTCCAGTGC |
| | | CTGGGCTTCACCCCTGAACATCAGCGAGACTTCATTGCCCGTG |
| | | ACCTAGGTCCTACCCTCGCCAACAGTACTCACCACAATGTCCG |
| | | CCTACTCATGCTGGATGACCAACGCTTGCTGCTGCCCCACTGGG |
| | | CAAAGGTGGTACTGACAGACCCAGAAGCAGCTAAATATGTTCA |
| | | TGGCATTGCTGTACATTGGTACCTGGACTTTCTGGCTCCAGCCA |
| | | AAGCCACCCTAGGGGAGACACACCGCCTGTTCCCCAACACCAT |
| | | GCTCTTTGCCTCAGAGGCCTGTGTGGGCTCCAAGTTCTGGGAGC |
| | | AGAGTGTGCGGCTAGGCTCCTGGGATCGAGGGATGCAGTACAG |
| | | CCACAGCATCATCACGAACCTCCTGTACCATGTGGTCGGCTGG |
| | | ACCGACTGGAACCTTGCCCTGAACCCCGAAGGAGGACCCAATT |
| | | GGGTGCGTAACTTTGTCGACAGTCCCATCATTGTAGACATCACC |
| | | AAGGACACGTTTTACAAACAGCCCATGTTCTACCACCTTGGCC |
| | | ACTTCAGCAAGTTCATTCCTGAGGGCTCCCAGAGAGTGGGGCT |
| | | GGTTGCCAGTCAGAAGAACGACCTGGACGCAGTGGCACTGATG |
| | | CATCCCGATGGCTCTGCTGTTGTGGTCGTGCTAAACCGCTCCTC |
| | | TAAGGATGTGCCTCTTACCATCAAGGATCCTGCTGTGGGCTTCC |
| | | TGGAGACAATCTCACCTGGCTACTCCATTCACACCTACCTGTGG |
| | | CGTCGCCAGTGATGGAGCAGATACTCAAGGAGGCACTGGGCTC |
| | | AGCCTGGGCATTAAAGGGACAGAGTCAGCTCACACGCTGTCTG |
| | | TGACTAAAGAGGGCACAGCAGGGCCAGTGTGAGCTTACAGCG |
| | | ACGTAAGCCCAGGGGCAATGGTTTGGGTGACTCACTTTCCCCT |
| | | CTAGGTGGTGCCAGGGGCTGGAGGCCCCTAGAAAAAGATCAGT |
| | | AAGCCCCAGTGTCCCCCCAGCCCCCATGCTTATGTGAACATGC |
| | | GCTGTGTGCTGCTTGCTTTGGAAACTGGGCCTGGGTCCAGGCCT |
| | | AGGGTGAGCTCACTGTCCGTACAAACACAAGATCAGGGCTGAG |
| | | GGTAAGGAAAAGAAGAGACTAGGAAAGCTGGGCCCAAAACTG |
| | | GAGACTGTTTGTCTTTCCTGGAGATGCAGAACTGGGCCCGTGG |
| | | AGCAGCAGTGTCAGCATCAGGGCGGAAGCCTTAAAGCAGCAG |
| | | CGGGTGTGCCCAGGCACCCAGATGATTCCTATGGCACCAGCCA |
| | | GGAAAAATGGCAGCTCTTAAAGGAGAAAATGTTTGAGCCCA |
| 51 | 2 | ATCCTGCCTTCAGAGTCTTACTGCGCGGGGCCCCAGTCTCCAGT |
| | | CCCGCCCAGGCGCCTTTGCAGGCTGCGGTGGGATTTCGTTTTGC |
| | | CTCCGGTTGGGGCTGCTGTTTCTCTTCGCCGACGTGGATCCTCT |
| | | ATCCTTCAGAGACTCTGGAACCCCTGTGGTCTTCTCTTCATCTA |
| | | ATGACCCTGAGGGGATGGAGTTTTCAAGTCCTTCCAGAGAGGA |
| | | ATGTCCCAAGCCTTTGAGTAGGGTAAGCATCATGGCTGGCAGC |
| | | CTCACAGGATTGCTTCTACTTCAGGCAGTGTCGTGGGCATCAG |
| | | GTGCCCGCCCCTGCATCCCTAAAAGCTTCGGCTACAGCTCGGT |
| | | GGTGTGTGTCTGCAATGCCACATACTGTGACTCCTTTGACCCCC |
| | | CGACCTTTCCTGCCCTTGGTACCTTCAGCCGCTATGAGAGTACA |
| | | CGCAGTGGGCGACGGATGGAGCTGAGTATGGGGCCCATCCAG |
| | | GCTAATCACACGGGCACAGGCCTGCTACTGACCCTGCAGCCAG |
| | | AACAGAAGTTCCAGAAAGTGAAGGGATTTGGAGGGGCCATGA |
| | | CAGATGCTGCTGCTCTCAACATCCTTGCCCTGTCACCCCCTGCC |
| | | CAAAATTTGCTACTTAAATCGTACTTCTCTGAAGAAGGAATCG |
| | | GATATAACATCATCCGGGTACCCATGGCCAGCTGTGACTTCTCC |
| | | ATCCGCACCTACACCTATGCAGACACCCCTGATGATTTCCAGTT |
| | | GCACAACTTCAGCCTCCCAGAGGAAGATACCAAGCTCAAGATA |
| | | CCCCTGATTCACCGAGCCCTGCAGTTGGCCCAGCGTCCCGTTTC |
| | | ACTCCTTGCCAGCCCCTGGACATCACCCACTTGGCTCAAGACC |
| | | AATGGAGCGGTGAATGGGAAGGGGTCACTCAAGGGACAGCCC |
| | | GGAGACATCTACCACCAGACCTGGGCCAGATACTTTGTGAAGT |
| | | TCCTGGATGCCTATGCTGAGCACAAGTTACAGTTCTGGGCAGT |
| | | GACAGCTGAAAATGAGCCTTCTGCTGGGCTGTTGAGTGGATAC |
| | | CCCTTCCAGTGCCTGGGCTTCACCCCTGAACATCAGCGAGACTT |
| | | CATTGCCCGTGACCTAGGTCCTACCCTCGCCAACAGTACTCACC |
| | | ACAATGTCCGCCTACTCATGCTGGATGACCAACGCTTGCTGCTG |
| | | CCCCACTGGGCAAAGGTGGTACTGACAGACCCAGAAGCAGCTA |
| | | AATATGTTCATGGCATTGCTGTACATTGGTACCTGGACTTTCTG |
| | | GCTCCAGCCAAAGCCACCCTAGGGGAGACACACCGCCTGTTCC |
| | | CCAACACCATGCTCTTTGCCTCAGAGGCCTGTGTGGGCTCCAA |
| | | GTTCTGGGAGCAGAGTGTGCGGCTAGGCTCCTGGGATCGAGGG |
| | | ATGCAGTACAGCCACAGCATCATCACGAACCTCCTGTACCATG |
| | | TGGTCGGCTGGACCGACTGGAACCTTGCCCTGAACCCCGAAGG |
| | | AGGACCCAATTGGGTGCGTAACTTTGTCGACAGTCCCATCATT |
| | | GTAGACATCACCAAGGACACGTTTTACAAACAGCCCATGTTCT |
| | | ACCACCTTGGCCACTTCAGCAAGTTCATTCCTGAGGGCTCCCAG |
| | | AGAGTGGGGCTGGTTGCCAGTCAGAAGAACGACCTGGACGCA |
| | | GTGGCACTGATGCATCCCGATGGCTCTGCTGTTGTGGTCGTGCT |
| | | AAACCGCTCCTCTAAGGATGTGCCTCTTACCATCAAGGATCCTG |
| | | CTGTGGGCTTCCTGGAGACAATCTCACCTGGCTACTCCATTCAC |
| | | ACCTACCTGTGGCGTCGCCAGTGATGGAGCAGATACTCAAGGA |
| | | GGCACTGGGCTCAGCCTGGGCATTAAAGGGACAGAGTCAGCTC |
| | | ACACGCTGTCTGTGACTAAAGAGGGCACAGCAGGGCCAGTGTG |
| | | AGCTTACAGCGACGTAAGCCCAGGGGCAATGGTTTGGGTGACT |
| | | CACTTTCCCCTCTAGGTGGTGCCAGGGGCTGGAGGCCCCTAGA |
| | | AAAAGATCAGTAAGCCCCAGTGTCCCCCCAGCCCCCATGCTTA |
| | | TGTGAACATGCGCTGTGTGCTGCTTGCTTTGGAAACTGGGCCTG |
| | | GGTCCAGGCCTAGGGTGAGCTCACTGTCCGTACAAACACAAGA |
| | | TCAGGGCTGAGGGTAAGGAAAAGAAGAGACTAGGAAAGCTGG |
| | | GCCCAAAACTGGAGACTGTTTGTCTTTCCTGGAGATGCAGAAC |
| | | TGGGCCCGTGGAGCAGCAGTGTCAGCATCAGGGCGGAAGCCTT |
| | | AAAGCAGCAGCGGGTGTGCCCAGGCACCCAGATGATTCCTATG |
| | | GCACCAGCCAGGAAAAATGGCAGCTCTTAAAGGAGAAAATGT |
| | | TTGAGCCCA |
| 52 | 3 | ATCCTGCCTTCAGAGTCTTACTGCGCGGGGCCCCAGTCTCCAGT |
| | | CCCGCCCAGGCGCCTTTGCAGGCTGCGGTGGGATTTCGTTTTGC |
| | | CTCCGGTTGGGGCTGCTGTTTCTCTTCGCCGACGAGACTCTGGA |
| | | ACCCCTGTGGTCTTCTCTTCATCTAATGACCCTGAGGGGATGGA |
| | | GTTTTCAAGTCCTTCCAGAGAGGAATGTCCCAAGCCTTTGAGTA |
| | | GGGTAAGCATCATGGCTGGCAGCCTCACAGGATTGCTTCTACT |
| | | TCAGGCAGTGTCGTGGGCATCAGGTGCCCGCCCCTGCATCCCT |
| | | AAAAGCTTCGGCTACAGCTCGGTGGTGTGTGTCTGCAATGCCA |
| | | CATACTGTGACTCCTTTGACCCCCCGACCTTTCCTGCCCTTGGT |
| | | ACCTTCAGCCGCTATGAGAGTACACGCAGTGGGCGACGGATGG |
| | | AGCTGAGTATGGGGCCCATCCAGGCTAATCACACGGGCACAGG |
| | | CCTGCTACTGACCCTGCAGCCAGAACAGAAGTTCCAGAAAGTG |
| | | AAGGGATTTGGAGGGGCCATGACAGATGCTGCTGCTCTCAACA |
| | | TCCTTGCCCTGTCACCCCCTGCCCAAAATTTGCTACTTAAATCG |
| | | TACTTCTCTGAAGAAGGAATCGGATATAACATCATCCGGGTAC |
| | | CCATGGCCAGCTGTGACTTCTCCATCCGCACCTACACCTATGCA |
| | | GACACCCCTGATGATTTCCAGTTGCACAACTTCAGCCTCCCAGA |
| | | GGAAGATACCAAGCTCAAGATACCCCTGATTCACCGAGCCCTG |
| | | CAGTTGGCCCAGCGTCCCGTTTCACTCCTTGCCAGCCCCTGGAC |
| | | ATCACCCACTTGGCTCAAGACCAATGGAGCGGTGAATGGGAAG |
| | | GGGTCACTCAAGGGACAGCCCGGAGACATCTACCACCAGACCT |
| | | GGGCCAGATACTTTGTGAAGTTCCTGGATGCCTATGCTGAGCA |
| | | CAAGTTACAGTTCTGGGCAGTGACAGCTGAAAATGAGCCTTCT |
| | | GCTGGGCTGTTGAGTGGATACCCCTTCCAGTGCCTGGGCTTCAC |
| | | CCCTGAACATCAGCGAGACTTCATTGCCCGTGACCTAGGTCCT |
| | | ACCCTCGCCAACAGTACTCACCACAATGTCCGCCTACTCATGCT |
| | | GGATGACCAACGCTTGCTGCTGCCCCACTGGGCAAAGGTGGTA |
| | | CTGACAGACCCAGAAGCAGCTAAATATGTTCATGGCATTGCTG |
| | | TACATTGGTACCTGGACTTTCTGGCTCCAGCCAAAGCCACCCTA |
| | | GGGGAGACACACCGCCTGTTCCCCAACACCATGCTCTTTGCCTC |
| | | AGAGGCCTGTGTGGGCTCCAAGTTCTGGGAGCAGAGTGTGCGG |
| | | CTAGGCTCCTGGGATCGAGGGATGCAGTACAGCCACAGCATCA |
| | | TCACGAACCTCCTGTACCATGTGGTCGGCTGGACCGACTGGAA |
| | | CCTTGCCCTGAACCCCGAAGGAGGACCCAATTGGGTGCGTAAC |
| | | TTTGTCGACAGTCCCATCATTGTAGACATCACCAAGGACACGTT |
| | | TTACAAACAGCCCATGTTCTACCACCTTGGCCACTTCAGCAAGT |
| | | TCATTCCTGAGGGCTCCCAGAGAGTGGGGCTGGTTGCCAGTCA |
| | | GAAGAACGACCTGGACGCAGTGGCACTGATGCATCCCGATGGC |
| | | TCTGCTGTTGTGGTCGTGCTAAACCGCTCCTCTAAGGATGTGCC |
| | | TCTTACCATCAAGGATCCTGCTGTGGGCTTCCTGGAGACAATCT |
| | | CACCTGGCTACTCCATTCACACCTACCTGTGGCGTCGCCAGTGA |
| | | TGGAGCAGATACTCAAGGAGGCACTGGGCTCAGCCTGGGCATT |
| | | AAAGGGACAGAGTCAGCTCACACGCTGTCTGTGACTAAAGAGG |
| | | GCACAGCAGGGCCAGTGTGAGCTTACAGCGACGTAAGCCCAG |
| | | GGGCAATGGTTTGGGTGACTCACTTTCCCCTCTAGGTGGTGCCA |
| | | GGGGCTGGAGGCCCCTAGAAAAAGATCAGTAAGCCCCAGTGTC |
| | | CCCCCAGCCCCCATGCTTATGTGAACATGCGCTGTGTGCTGCTT |
| | | GCTTTGGAAACTGGGCCTGGGTCCAGGCCTAGGGTGAGCTCAC |
| | | TGTCCGTACAAACACAAGATCAGGGCTGAGGGTAAGGAAAAG |
| | | AAGAGACTAGGAAAGCTGGGCCCAAAACTGGAGACTGTTTGTC |
| | | TTTCCTGGAGATGCAGAACTGGGCCCGTGGAGCAGCAGTGTCA |
| | | GCATCAGGGCGGAAGCCTTAAAGCAGCAGCGGGTGTGCCCAG |
| | | GCACCCAGATGATTCCTATGGCACCAGCCAGGAAAAATGGCAG |
| | | CTCTTAAAGGAGAAAATGTTTGAGCCCA |
| 53 | 4 | ATCCTGCCTTCAGAGTCTTACTGCGCGGGGCCCCAGTCTCCAGT |
| | | CCCGCCCAGGCGCCTTTGCAGGCTGCGGTGGGATTTCGTTTTGC |
| | | CTCCGGTTGGGGCTGCTGTTTCTCTTCGCCGACGGTGCCCGCCC |
| | | CTGCATCCCTAAAAGCTTCGGCTACAGCTCGGTGGTGTGTGTCT |
| | | GCAATGCCACATACTGTGACTCCTTTGACCCCCCGACCTTTCCT |
| | | GCCCTTGGTACCTTCAGCCGCTATGAGAGTACACGCAGTGGGC |
| | | GACGGATGGAGCTGAGTATGGGGCCCATCCAGGCTAATCACAC |
| | | GGGCACAGGCCTGCTACTGACCCTGCAGCCAGAACAGAAGTTC |
| | | CAGAAAGTGAAGGGATTTGGAGGGGCCATGACAGATGCTGCT |
| | | GCTCTCAACATCCTTGCCCTGTCACCCCCTGCCCAAAATTTGCT |
| | | ACTTAAATCGTACTTCTCTGAAGAAGGAATCGGATATAACATC |
| | | ATCCGGGTACCCATGGCCAGCTGTGACTTCTCCATCCGCACCTA |
| | | CACCTATGCAGACACCCCTGATGATTTCCAGTTGCACAACTTCA |
| | | GCCTCCCAGAGGAAGATACCAAGCTCAAGATACCCCTGATTCA |
| | | CCGAGCCCTGCAGTTGGCCCAGCGTCCCGTTTCACTCCTTGCCA |
| | | GCCCCTGGACATCACCCACTTGGCTCAAGACCAATGGAGCGGT |
| | | GAATGGGAAGGGGTCACTCAAGGGACAGCCCGGAGACATCTA |
| | | CCACCAGACCTGGGCCAGATACTTTGTGAAGTTCCTGGATGCC |
| | | TATGCTGAGCACAAGTTACAGTTCTGGGCAGTGACAGCTGAAA |
| | | ATGAGCCTTCTGCTGGGCTGTTGAGTGGATACCCCTTCCAGTGC |
| | | CTGGGCTTCACCCCTGAACATCAGCGAGACTTCATTGCCCGTG |
| | | ACCTAGGTCCTACCCTCGCCAACAGTACTCACCACAATGTCCG |
| | | CCTACTCATGCTGGATGACCAACGCTTGCTGCTGCCCCACTGGG |
| | | CAAAGGTGGTACTGACAGACCCAGAAGCAGCTAAATATGTTCA |
| | | TGGCATTGCTGTACATTGGTACCTGGACTTTCTGGCTCCAGCCA |
| | | AAGCCACCCTAGGGGAGACACACCGCCTGTTCCCCAACACCAT |
| | | GCTCTTTGCCTCAGAGGCCTGTGTGGGCTCCAAGTTCTGGGAGC |
| | | AGAGTGTGCGGCTAGGCTCCTGGGATCGAGGGATGCAGTACAG |
| | | CCACAGCATCATCACGAACCTCCTGTACCATGTGGTCGGCTGG |
| | | ACCGACTGGAACCTTGCCCTGAACCCCGAAGGAGGACCCAATT |
| | | GGGTGCGTAACTTTGTCGACAGTCCCATCATTGTAGACATCACC |
| | | AAGGACACGTTTTACAAACAGCCCATGTTCTACCACCTTGGCC |
| | | ACTTCAGCAAGTTCATTCCTGAGGGCTCCCAGAGAGTGGGGCT |
| | | GGTTGCCAGTCAGAAGAACGACCTGGACGCAGTGGCACTGATG |
| | | CATCCCGATGGCTCTGCTGTTGTGGTCGTGCTAAACCGCTCCTC |
| | | TAAGGATGTGCCTCTTACCATCAAGGATCCTGCTGTGGGCTTCC |
| | | TGGAGACAATCTCACCTGGCTACTCCATTCACACCTACCTGTGG |
| | | CGTCGCCAGTGATGGAGCAGATACTCAAGGAGGCACTGGGCTC |
| | | AGCCTGGGCATTAAAGGGACAGAGTCAGCTCACACGCTGTCTG |
| | | TGACTAAAGAGGGCACAGCAGGGCCAGTGTGAGCTTACAGCG |
| | | ACGTAAGCCCAGGGGCAATGGTTTGGGTGACTCACTTTCCCCT |
| | | CTAGGTGGTGCCAGGGGCTGGAGGCCCCTAGAAAAAGATCAGT |
| | | AAGCCCCAGTGTCCCCCCAGCCCCCATGCTTATGTGAACATGC |
| | | GCTGTGTGCTGCTTGCTTTGGAAACTGGGCCTGGGTCCAGGCCT |
| | | AGGGTGAGCTCACTGTCCGTACAAACACAAGATCAGGGCTGAG |
| | | GGTAAGGAAAAGAAGAGACTAGGAAAGCTGGGCCCAAAACTG |
| | | GAGACTGTTTGTCTTTCCTGGAGATGCAGAACTGGGCCCGTGG |
| | | AGCAGCAGTGTCAGCATCAGGGCGGAAGCCTTAAAGCAGCAG |
| | | CGGGTGTGCCCAGGCACCCAGATGATTCCTATGGCACCAGCCA |
| | | GGAAAAATGGCAGCTCTTAAAGGAGAAAATGTTTGAGCCCA |
| 54 | 5 | GGAATTACTTGCAGGGCTAACCTAGTGCCTATAGCTAAGGCAG |
| | | GTACCTGCATCCTTGTTTTTGTTTAGTGGATCCTCTATCCTTCAG |
| | | AGACTCTGGAACCCCTGTGGTCTTCTCTTCATCTAATGACCCTG |
| | | AGGGGATGGAGTTTTCAAGTCCTTCCAGAGAGGAATGTCCCAA |
| | | GCCTTTGAGTAGGGTAAGCATCATGGCTGGCAGCCTCACAGGA |
| | | TTGCTTCTACTTCAGGCAGTGTCGTGGGCATCAGGTGCCCGCCC |
| | | CTGCATCCCTAAAAGCTTCGGCTACAGCTCGGTGGTGTGTGTCT |
| | | GCAATGCCACATACTGTGACTCCTTTGACCCCCCGACCTTTCCT |
| | | GCCCTTGGTACCTTCAGCCGCTATGAGAGTACACGCAGTGGGC |
| | | GACGGATGGAGCTGAGTATGGGGCCCATCCAGGCTAATCACAC |
| | | GGGCACAGGAATCGGATATAACATCATCCGGGTACCCATGGCC |
| | | AGCTGTGACTTCTCCATCCGCACCTACACCTATGCAGACACCCC |
| | | TGATGATTTCCAGTTGCACAACTTCAGCCTCCCAGAGGAAGAT |
| | | ACCAAGCTCAAGATACCCCTGATTCACCGAGCCCTGCAGTTGG |
| | | CCCAGCGTCCCGTTTCACTCCTTGCCAGCCCCTGGACATCACCC |
| | | ACTTGGCTCAAGACCAATGGAGCGGTGAATGGGAAGGGGTCA |
| | | CTCAAGGGACAGCCCGGAGACATCTACCACCAGACCTGGGCCA |
| | | GATACTTTGTGAAGTTCCTGGATGCCTATGCTGAGCACAAGTTA |
| | | CAGTTCTGGGCAGTGACAGCTGAAAATGAGCCTTCTGCTGGGC |
| | | TGTTGAGTGGATACCCCTTCCAGTGCCTGGGCTTCACCCCTGAA |
| | | CATCAGCGAGACTTCATTGCCCGTGACCTAGGTCCTACCCTCGC |
| | | CAACAGTACTCACCACAATGTCCGCCTACTCATGCTGGATGAC |
| | | CAACGCTTGCTGCTGCCCCACTGGGCAAAGGTGGTACTGACAG |
| | | ACCCAGAAGCAGCTAAATATGTTCATGGCATTGCTGTACATTG |
| | | GTACCTGGACTTTCTGGCTCCAGCCAAAGCCACCCTAGGGGAG |
| | | ACACACCGCCTGTTCCCCAACACCATGCTCTTTGCCTCAGAGGC |
| | | CTGTGTGGGCTCCAAGTTCTGGGAGCAGAGTGTGCGGCTAGGC |
| | | TCCTGGGATCGAGGGATGCAGTACAGCCACAGCATCATCACGA |
| | | ACCTCCTGTACCATGTGGTCGGCTGGACCGACTGGAACCTTGC |
| | | CCTGAACCCCGAAGGAGGACCCAATTGGGTGCGTAACTTTGTC |
| | | GACAGTCCCATCATTGTAGACATCACCAAGGACACGTTTTACA |
| | | AACAGCCCATGTTCTACCACCTTGGCCACTTCAGCAAGTTCATT |
| | | CCTGAGGGCTCCCAGAGAGTGGGGCTGGTTGCCAGTCAGAAGA |
| | | ACGACCTGGACGCAGTGGCACTGATGCATCCCGATGGCTCTGC |
| | | TGTTGTGGTCGTGCTAAACCGCTCCTCTAAGGATGTGCCTCTTA |
| | | CCATCAAGGATCCTGCTGTGGGCTTCCTGGAGACAATCTCACCT |
| | | GGCTACTCCATTCACACCTACCTGTGGCGTCGCCAGTGATGGA |
| | | GCAGATACTCAAGGAGGCACTGGGCTCAGCCTGGGCATTAAAG |
| | | GGACAGAGTCAGCTCACACGCTGTCTGTGACTAAAGAGGGCAC |
| | | AGCAGGGCCAGTGTGAGCTTACAGCGACGTAAGCCCAGGGGC |
| | | AATGGTTTGGGTGACTCACTTTCCCCTCTAGGTGGTGCCAGGGG |
| | | CTGGAGGCCCCTAGAAAAAGATCAGTAAGCCCCAGTGTCCCCC |
| | | CAGCCCCCATGCTTATGTGAACATGCGCTGTGTGCTGCTTGCTT |
| | | TGGAAACTGGGCCTGGGTCCAGGCCTAGGGTGAGCTCACTGTC |
| | | CGTACAAACACAAGATCAGGGCTGAGGGTAAGGAAAAGAAGA |
| | | GACTAGGAAAGCTGGGCCCAAAACTGGAGACTGTTTGTCTTTC |
| | | CTGGAGATGCAGAACTGGGCCCGTGGAGCAGCAGTGTCAGCAT |
| | | CAGGGCGGAAGCCTTAAAGCAGCAGCGGGTGTGCCCAGGCAC |
| | | CCAGATGATTCCTATGGCACCAGCCAGGAAAAATGGCAGCTCT |
| | | TAAAGGAGAAAATGTTTGAGCCCA |

### Genome Editing of LRRK2

In some embodiments, the LRRK2 gene can be altered using genome editing. Genome editing can comprise a CRISPR/Cas associated protein, RNA guided endonuclease, zinc finger nuclease, transcription activator-like effector nuclease (TALEN), meganuclease, functional portion of any of these, fusion protein of any of these, or any combination thereof. In some embodiments, a CRISPR/Cas associated protein can comprise a CRISPR/Cas endonuclease. In some embodiments, a CRISPR/Cas associated protein can comprise class 1 or class 2 CRISPR/Cas protein. A class 2 CRISPR/Cas associated protein can comprise a type II CRISPR/Cas protein, a type V CRISPR/Cas protein, a type VI CRISPR/Cas protein. A CRISPR/Cas associated protein can comprise a Cas9 protein, Cas 12 protein, Cas13 protein, functional portion of any of these, fusion protein of any of these, or any combinations thereof. A CRISPR/Cas associated protein can comprise a wildtype or a variant CRISPR/Cas associated protein, functional portion of any of these, fusion protein of any of these, or any combinations thereof. A CRISPR/Cas associated protein can comprise a base editor. A base editor can comprise a cytidine deaminase, a deoxyadenosine deaminase, functional portion of any of these, fusion protein of any of these, or any combinations thereof. A CRISPR/Cas associated protein can comprise a reverse transcriptase. A reverse transcriptase can comprise a Moloney murine leukemia virus (M-MLV) reverse transcriptase or an Avian Myeloblastosis Virus (AMV) reverse transcriptase.

A CRISPR/Cas associated protein as described herein are targeted to a specific target DNA sequence in a genome by a guide RNA to which it is bound. The guide RNA comprises a sequence that is complementary to a target sequence within the target DNA, thus targeting the bound CRISPR/Cas protein to a specific location within the target DNA (the target sequence). A CRISPR/Cas associated protein, when targeted to the specific target DNA sequence, can create a single-strand break, two single-strand breaks, a double-strand break, two double-strand breaks, or any combinations thereof in the genome. A CRISPR/Cas associated protein, when targeted to the specific target DNA sequence, may not create any breaks in the genome. A CRISPR/Cas associated protein-guide RNA complex can make a blunt-ended double-stranded break, a 1-base pair (bp) staggered cut, a 2-bp staggered cut, a staggered cut with more than 2 base pairs, or any combination thereof in the genome. A double-strand DNA break can be repaired by end-joining mechanism or homologous directed repair. A double-strand DNA break can also be repaired by end-joining mechanism or homologous directed repair with a double strand donor DNA or a single-stranded oligonucleotide donor DNA. An edit in the genome can comprise stochastic or pre-selected insertions, deletions, base substitutions, inversion, chromosomal translocation, insertion.

A guide RNA can comprise a single guide RNA (sgRNA), a double guide RNA, or an engineered prime editing guide RNA (pegRNA). A guide RNA can comprise a crRNA and a tracrRNA. A crRNA can comprise a targeting sequence that hybridizes to a target sequence in the target DNA or locus. A tracrRNA can comprise a sequence that can form a stem-loop structure. Such a stem-loop structure can bind a CRISPR/Cas associated protein to activate the nuclease activity of the CRISPR/Cas associated protein. A sgRNA can comprise a crRNA and a tracrRNA in one RNA molecule. A double guide RNA can comprise a crRNA and a tracrRNA in two RNA molecules. A pegRNA can comprise a sequence that comprises a pre-selected edit or sequence in the genome. In such editing, the pre-selected sequence hybridizes to a cut and liberated 3′ end of a nicked/cut DNA strand to form a primer-template complex, wherein the cut, liberated, and hybridized 3′ end of the nicked/cut DNA strand can serve as a primer while the pre-selected edit or sequence of the pegRNA can serve as a template for the subsequent reactions, including but not limited to reverse transcription.

### Vectors

The compositions provided herein (e.g., engineered polynucleotides) can be delivered by any suitable means. In some cases, a suitable means comprises a vector. Any vector system can be used utilized, including but not limited to: plasmid vectors, minicircle vectors, linear DNA vectors, doggy bone vectors, retroviral vectors, lentiviral vectors, adenovirus vectors, poxvirus vectors, herpesvirus vectors, adeno-associated virus (AAV) vectors, a liposome, a nanoparticle, an exosome, an extracellular vesicle, a nanomesh, modified versions thereof, good manufacturing practices versions thereof, chimeras thereof, and any combination thereof. In some cases, a vector can be used to introduce a polynucleotide provided herein. In some embodiments, a nanoparticle vector can comprise a polymeric-based nanoparticle, an aminolipid-based nanoparticle, a metallic nanoparticle (such as gold-based nanoparticle), a portion of any of these, or any combination thereof. In some cases, the polynucleotide (e.g., the engineered polynucleotide) delivered by the vector comprises a targeting sequence that hybridizes to a region of a target RNA provided herein.

Vectors provided herein can be used to deliver polynucleotide compositions provided herein. In some cases, at least about 2, 3, 4, or up to 5 polynucleotides are delivered using a single vector. In some cases, at least about 2, 3, 4, or up to 5 different polynucleotides are delivered using a single vector. In some cases, at least about 2, 3, 4, or up to 5 of the same polynucleotide are delivered using a single vector. In some cases, multiple vectors are delivered. In some cases, multiple vector delivery can be co-current or sequential.

A vector can be employed to deliver a nucleic acid. A vector can comprise DNA, such as double stranded DNA or single stranded DNA. A vector can comprise RNA. In some cases, the RNA can comprise a base modification. The vector can comprise a recombinant vector. The vector can be a vector that is modified from a naturally occurring vector. The vector can comprise at least a portion of a non-naturally occurring vector. Any vector can be utilized. A viral vector can comprise an adenoviral vector, an adeno-associated viral vector (AAV), a lentiviral vector, a retroviral vector, a portion of any of these, or any combination thereof. In some cases, a vector can comprise an AAV vector. A vector can be modified to include a modified VP protein (such as an AAV vector modified to include a VP1 protein, VP2 protein, or VP3 protein). In an aspect, an AAV vector is a recombinant AAV (rAAV) vector. rAAVs can be composed of substantially similar capsid sequence and structure as found in wild-type AAVs (wtAAVs). However, rAAVs encapsidate genomes that are substantially devoid of AAV protein-coding sequences and have therapeutic gene expression cassettes, such as subject polynucleotides, designed in their place. In some cases, sequences of viral origin can be the ITRs, which may be needed to guide genome replication and packaging during vector production. Suitable AAV vectors can be selected from any AAV serotype or combination of serotypes. For example, an AAV vector can be any one of: AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68, or any combination thereof. In some cases, a vector is selected based on its natural tropism. In some cases, a vector serotype is selected based on its ability to cross the blood brain barrier. AAV9 and AAV10 have been shown to cross the blood brain barrier to transduce neurons and glia. In an aspect, an AAV vector is AAV2, AAV5, AAV6, AAV8, or AAV9. In some cases, an AAV vector is a chimera of at least two serotypes. In an aspect, an AAV vector is of serotypes AAV2 and AAV5. In some cases, a chimeric AAV vector comprises rep and ITR sequences from AAV2 and a cap sequence from AAV5. In some cases, a chimeric AAV vector comprises rep and ITR sequences from AAV2 and a cap sequence from any other AAV serotype. In some embodiments, an AAV vector can be self-complementary. In some cases, an AAV vector can comprise an inverted terminal repeat. In other cases, an AAV vector can comprise an inverted terminal repeat (scITR) sequence with a mutated terminal resolution site. In some cases, rep, cap, and ITR sequences can be mixed and matched from all the of the different AAV serotypes provided herein. In some cases, an AAV vector is from an adeno-associated virus having a serotype selected from AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. In some cases, a vector can be a recombinant AAV (rAAV) vector, a hybrid AAV vector, a chimeric AAV vector, a self-complementary AAV (scAAV) vector, a single-stranded AAV or any combination thereof. In some cases, an AAV vector comprises a genome comprising a replication gene and inverted terminal repeats from a first AAV serotype and a capsid protein from a second AAV serotype. In some cases, an AAV vector can be chimeric and can be an: AAV 2/5 vector, an AAV 2/6 vector, an AAV 2/7 vector, an AAV2/8 vector, or an AAV 2/9 vector. In some cases, inverted terminal repeats of an AAV vector comprise a 5′ inverted terminal repeat, a 3′ inverted terminal repeat, and a mutated inverted terminal repeat. In some cases, mutated inverted terminal repeat lack a terminal resolution site. In some cases, a suitable AAV vector can be further modified to encompass modifications such as in a capsid or rep protein. Modifications can also include deletions, insertions, mutations, and combinations thereof. In some cases, a modification to a vector is made to reduce immunogenicity to allow for repeated dosing. In some cases, a serotype of a vector that is utilized is changed when repeated dosing is performed to reduce and/or eliminate immunogenicity.

In some embodiments, an AAV vector can comprise from 2 to 6 copies of engineered polynucleotides per viral genome. In some cases, an AAV vector can comprise from 1 to 2, from 1 to 3, from 1 to 4, from 1 to 5, from 1 to 6, from 1 to 7, from 1 to 8, from 1 to 9, from 1 to 10, from 2 to 3, from 2 to 4, from 2 to 5, from 2 to 6, from 2 to 7, from 2 to 8, from 2 to 9, or from 2 to 10 copies per viral genome. In some cases, an AAV vector can comprise 1, 2, 3, 4, 5, 6, 7, 8, 9, or 10 copies per viral genome. In some embodiments, an AAV vector can comprise from 1 to 5, from 1 to 10, from 1 to 15, from 1 to 20, from 1 to 25, from 1 to 30, from 1 to 35, from 1 to 40, from 1 to 45, or from 1 to 50 copies per viral genome.

Vectors can be delivered in vivo by administration to a subject, typically by systemic administration (e.g., intravenous, intraparenchymal, intraperitoneal, intramuscular, subdermal, or intracranial infusion) or topical application, or a combination thereof. Various administrations can be made. In some cases, administration of a vector is performed 1, 2, 3, 4, 5, 6, 7, 8, 9, or 10 times. Frequency of administration can also be modulated. In an aspect, a vector provided herein is administered hourly, daily, weekly, monthly, bi monthly, yearly, biyearly, or every 2, 4, 6, or 8 years.

In some cases, a vector provided herein can integrate into a genome of a subject. This may be useful in achieving prolonged expression of transgene expression and/or polypeptide expression.

Vectors provided herein can be utilized to transfect a target cell. Target cells can be found in any of tissues and organs of the body. In some cases, a target cell is found in a tissue or organ implicated in a disease. A disease can be of the CNS or of the gastrointestinal tract. In some cases, a disease can be Parkinson's and/or Crohn's disease. In some cases, the disease can be Lewy body dementia, multiple system atrophy (MSA), Gaucher disease, Alzheimer's disease, frontotemporal dementia (FTD), chronic traumatic encephalopathy (CTE), progressive supranuclear palsy, or corticobasal degeneration.

Suitable target cells for the treatment of Parkinson's disease can include neurons or glia cells. Suitable target neurons for the treatment of Parkinson's disease can include dopaminergic (DA) neurons or norepinephrine (NE) neurons. Suitable target dopaminergic neurons for the treatment of Parkinson's disease can include dopaminergic neurons in the ventral mesencephalon. Suitable target dopaminergic neurons for the treatment of Parkinson's disease can also include group A8, A9, A10, A11, A12, A13, A14, A15, A16, Aaq, or Telencephalic group dopaminergic neurons. Suitable target glial cells for the treatment of Parkinson's disease can include astrocytes, ependymal cells, microglial cells, oligodendrocytes, satellite cells, or Schwann cells. Suitable target microglial cells for the treatment of Parkinson's disease can include compact, longitudinally branched, or radially branched microglial cells.

Suitable target cells for the treatment of Crohn's are: dendritic cells, eosinophils, intraepithelial lymphocytes, macrophages, mast cells, neutrophils, or T-reg cells.

In some instances, a cell subjected to a treatment can comprise a human cell. In some cases, a cell subjected to a treatment can comprise a leukocyte. In some embodiments, a cell subjected to a treatment can comprise a lymphocyte. In some instances, a cell subjected to a treatment can comprise a T-cell. In some case, a cell subjected to a treatment can comprise a helper CD4+ T-cell, a cytotoxic CD8+ T-cell, a memory T-cell, a regulatory CD4+ T-cell, a natural killer T-cell, a mucosal associated T-cell, a gamma delta T-cell, or any combination thereof. In some embodiments, a cell subjected to a treatment can comprise a B-cell. In some cases, a cell subjected to a treatment can comprise a plasmablast, a plasma cell, a lymphoplasmacytoid cell, a memory B-cell, a follicular B-cell, a marginal zone B-cell, a B-1 cell, a regulatory B cell, or any combination thereof.

Suitable target cells for the treatment of a CNS disease can include neurons or glia cells.

In some cases, the transfection efficiency or editing efficiency of target cells with any of the vectors encoding polynucleotides and/or naked polynucleotides described herein, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%. Transfection efficiency or editing efficiency can be determined by evaluating disease burden. Transfection efficiency can also be determined by evaluating reduction in disease symptoms. In some cases, an editing efficiency can be therapeutically effective, meaning that editing achieves levels that can result in phenotypic changes in a treated subject. Phenotypic changes can comprise reduction or elimination of disease as measured by level of a symptom associated with a mutation.

### Non-Viral Vector Approaches

In some cases, compositions provided herein can be delivered without a vector. Non-viral methods can comprise naked delivery of compositions comprising polynucleotides and the like. In some cases, modifications provided herein can be incorporated into polynucleotides to increase stability and combat degradation when being delivered as naked polynucleotides. In other cases, a non-viral approach can harness use of nanoparticles, liposomes, and the like.

### Methods of Use

The compositions provided herein can be utilized in methods provided herein. In some cases, a method comprises at least partially preventing, reducing, and/or treating a disease or condition, or a symptom of a disease or condition. Methods of the disclosure can be performed in a subject. A subject can be a human or non-human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician).

In some cases, a disease is of the central nervous system (CNS). An exemplary CNS disease can be Parkinson's Disease.

Parkinson's disease is a progressive degenerative disorder that affects the motor system. Early symptoms comprise tremor, rigidity, slowness of movement, and difficulty walking. Cognitive and behavioral problems may also occur. Dementia becomes common in the late stages of the disease. Other symptoms comprise depression, anxiety, and problems in sensation, sleep, and emotion. Currently, there is no cure. The cause of Parkinson's Disease is unknown but involves both inherited and environmental factors. Other risk factors comprise age and sex.

Diagnosis of Parkinson's Disease can be based on symptoms such as tremor or the involuntary and rhythmic movements of the limbs and jaw; muscle rigidity or stiffness of the limbs, shoulders, or neck; loss of spontaneous movement; loss of automatic movement; posture; unsteady walk or balance; depression; or dementia. A physician can assess medical history and neurological examination. Magnetic resonance imaging (MM), positron emission tomography (PET), and single-photon emission computerized tomography (SPECT) scan such as dopamine transporter scan (DaTscan) can also be used to support the diagnosis.

Parkinson's Disease can be monitored by the Unified Parkinson Disease Rating Scale (UPDRS), Hoehn and Yahr staging, or the Schwab and England rating of activities of daily living.

In some embodiments, an engineered polynucleotide is used to treat Parkinson's Disease. In some embodiments, the engineered polynucleotide targets a region of a LRRK2 mRNA (e.g., correcting a mutation). In some embodiments, the engineered polynucleotide targets a region of an SNCA mRNA (e.g., resulting in a knockdown of SNCA). In some embodiments, the engineered polynucleotide targets a region of a MAPT mRNA. In some embodiments, the engineered polynucleotide targets a region of a PINK1 mRNA. In some embodiments, the engineered polynucleotide targets a region of a GBA mRNA. In some embodiments, one or more different engineered polynucleotides are used to treat Parkinson's disease. For example, an engineered polynucleotide that targets a region of a LRRK2 mRNA and an engineered polynucleotide that targets a region of a SNCA mRNA are used to treat Parkinson's disease. In some embodiments, an engineered polynucleotide that targets a region of a GBA mRNA and an engineered polynucleotide that targets a region of a SNCA mRNA are used to treat Parkinson's disease. In some embodiments, an engineered polynucleotide that targets a region of a PINK1 mRNA and an engineered polynucleotide that targets a region of a SNCA mRNA are used to treat Parkinson's disease. In some embodiments, an engineered polynucleotide that targets a region of a Tau mRNA and an engineered polynucleotide that targets a region of a SNCA mRNA are used to treat Parkinson's disease. In some embodiments, an engineered polynucleotide that targets a region of a LRRK2 mRNA, an engineered polynucleotide that targets a region of a Tau, and an engineered polynucleotide that targets a region of a SNCA mRNA are used to treat Parkinson's disease.

In some cases, a disease is a gastrointestinal (GI) disease. An exemplary GI disease can be Crohn's Disease. Crohn's Disease is a type of inflammatory bowel disease affecting GI tract. Crohn's Disease causes inflammation of the digestive tract leading to abdominal pain, fatigue, fever, diarrhea, malnutrition, mouth sores, and weight loss. The causes of Crohn's Disease are unknown; factors such as environment, immune system, and microbiota are suggested to be involved. There is no known cure for Crohn's Disease. Risk factors include age, ethnicity, heredity, nonsteroidal anti-inflammatory medications, and smoking.

Diagnosis of Crohn's Disease can be based on blood tests, colonoscopy, computerized tomography (CT) scan, MRI, capsule endoscopy, or balloon-assisted enteroscopy.

Crohn's Disease can be monitored by quality indicators. Quality indicators for Crohn's Disease can comprise Accountability measures of American gastroenterology Association, Improvement measures of Crohn's and Colitis Foundation, IBD centers for excellence (Spain) of Grupo Español de Trabajo en Enfermedad de Crohn y Colitis ulcerosa (National IBD Society of Spain), Aligns with international initiative of International Consortium for Health Outcomes Measurement, Metrics for Canadian IBD of Canadian Quality Improvement Measures, or 5 Process measures of poor quality care of “Choosing Wisely” (Canada). Quality indicators for Crohn's Disease can also comprise American Gastroenterology Association (AGA) IBD performance measures, the Crohn's & Colitis Foundation (CCFA) process and outcome measures, the International Consortium for Health Outcomes Measurement IBD standard set, ImproveCareNow, or IBD Qorus.

In some embodiments, an engineered polynucleotide is used to treat Crohn's Disease. In some embodiments, the engineered polynucleotide targets a region of a LRRK2 mRNA (e.g., correcting a mutation).

In some embodiments, an engineered polynucleotide is used to treat Lewy body dementia. In some embodiments, the engineered polynucleotide targets a region of a LRRK2 mRNA.

In some embodiments, an engineered polynucleotide is used to treat Multiple System atrophy (MSA). In some embodiments, the engineered polynucleotide targets a region of a SNCA.

In some embodiments, an engineered polynucleotide is used to treat Gaucher's disease. In some embodiments, the engineered polynucleotide targets a region of a GBA mRNA.

In some embodiments, an engineered polynucleotide is used to treat a Taupathy.

In some embodiments, an engineered polynucleotide is used to treat Alzheimer's disease, frontotemporal dementia, chronic traumatic encephalopathy, progressive supranuclear palsy, or corticobasal degeneration. In some embodiments, the engineered polynucleotide targets a region of a MAPT mRNA.

In some cases, the disease or condition is associated with a mutation in a DNA molecule or RNA molecule encoding ABCA4, AAT, SERPINA1, SERPINA1 E342K, HEXA, LRRK2, SNCA, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof. In some examples, a protein encoded for by a mutated DNA molecule or RNA molecule encoding ABCA4, AAT, SERPINA1, SERPINA1 E342K, HEXA, LRRK2, SNCA, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof. contributes to, at least in part, the pathogenesis or progression of a disease. In some examples, the mutation in the DNA or RNA molecule is relative to an otherwise identical reference DNA or RNA molecule.

### Pharmaceutical Compositions

Compositions and methods provided herein can utilize pharmaceutical compositions. The compositions described throughout can be formulated into a pharmaceutical and be used to treat a human or mammal, in need thereof, diagnosed with a disease. In some cases, pharmaceutical compositions can be used prophylactically.

Vectors of the disclosure can be administered at any suitable dose to subject. Suitable doses can be at least about 5×10⁷ to 50×10¹³ genome copies/mL. In some cases, suitable doses can be at least about 5×10⁷, 6×10⁷, 7×10⁷, 8×10⁷, 9×10⁷, 10×10⁷, 11×10⁷, 15×10⁷, 20×10⁷, 25×10⁷, 30×10⁷ or 50×10⁷ genome copies/mL. In some embodiments, suitable doses can be about 5×10⁷ to 6×10⁷, 6×10⁷ to 7×10⁷, 7×10⁷ to 8×10⁷, 8×10⁷ to 9×10⁷, 9×10⁷ to 10×10⁷, 10×10⁷ to 11×10⁷, 11×10⁷ to 15×10⁷, 15×10⁷ to 20×10⁷, 20×10⁷ to 25×10⁷, 25×10⁷ to 30×10⁷, 30×10⁷ to 50×10⁷, or 50×10⁷ to 100×10⁷ genome copies/mL. In some cases, suitable doses can be about 5×10⁷ to 10×10⁷, 10×10⁷ to 25×10⁷, or 25×10⁷ to 50×10⁷ genome copies/mL. In some cases, suitable doses can be at least about 5×10⁸, 6×10⁸, 7×10⁸, 8×10⁸, 9×10⁸, 10×10⁸, 11×10⁸, 15×10⁸, 20×10⁸, 25×10⁸, 30×10⁸ or 50×10⁸ genome copies/mL. In some embodiments, suitable doses can be about 5×10⁸ to 6×10⁸, 6×10⁸ to 7×10⁸, 7×10⁸ to 8×10⁸, 8×10⁸ to 9×10⁸, 9×10⁸ to 10×10⁸, 10×10⁸ to 11×10⁸, 11×10⁸ to 15×10⁸, 15×10⁸ to 20×10⁸, 20×10⁸ to 25×10⁸, 25×10⁸ to 30×10⁸, 30×10⁸ to 50×10⁸, or 50×10⁸ to 100×10⁸ genome copies/mL. In some cases, suitable doses can be about 5×10⁸ to 10×10⁸, 10×10⁸ to 25×10⁸, or 25×10⁸ to 50×10⁸ genome copies/mL. In some cases, suitable doses can be at least about 5×10⁹, 6×10⁹, 7×10⁹, 8×10⁹, 9×10⁹, 10×10⁹, 11×10⁹, 15×10⁹, 20×10⁹, 25×10⁹, 30×10⁹ or 50×10⁹ genome copies/mL. In some embodiments, suitable doses can be about 5×10⁹ to 6×10⁹, 6×10⁹ to 7×10⁹, 7×10⁹ to 8×10⁹, 8×10⁹ to 9×10⁹, 9×10⁹ to 10×10⁹, 10×10⁹ to 11×10⁹, 11×10⁹ to 15×10⁹, 15×10⁹ to 20×10⁹, 20×10⁹ to 25×10⁹, 25×10⁹ to 30×10⁹, 30×10⁹ to 50×10⁹, or 50×10⁹ to 100×10⁹ genome copies/mL. In some cases, suitable doses can be about 5×10⁹ to 10×10⁹, 10×10⁹ to 25×10⁹, or 25×10⁹ to 50×10⁹ genome copies/mL. In some cases, suitable doses can be at least about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰ or 50×10¹⁰ genome copies/mL. In some embodiments, suitable doses can be about 5×10¹⁰ to 6×10¹⁰, 6×10¹⁰ to 7×10¹⁰, 7×10¹⁰ to 8×10¹⁰, 8×10¹⁰ to 9×10¹⁰, 9×10¹⁰ to 10×10¹⁰, 10×10¹⁰ to 11×10¹⁰, 10×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 50×10¹⁰, or 50×10¹⁰ to 100×10¹⁰ genome copies/mL. In some cases, suitable doses can be about 5×10¹⁰ to 10×10¹⁰, 10×10¹⁰ to 25×10¹⁰, or 25×10¹⁰ to 50×10¹⁰ genome copies/mL. In some cases, suitable doses can be at least about 5×10¹¹, 6×10¹¹, 7×10¹¹, 8×10¹¹, 9×10¹¹, 10×10¹¹, 11×10¹¹, 15×10¹¹, 20×10¹¹, 25×10¹¹, 30×10¹¹ or 50×10¹¹ genome copies/mL. In some embodiments, suitable doses can be about 5×10¹¹ to 6×10¹¹, 6×10¹¹ to 7×10¹¹, 7×10¹¹ to 8×10¹¹, 8×10¹¹ to 9×10¹¹, 9×10¹¹ to 10×10¹¹, 10×10¹¹ to 11×10¹¹, 11×10¹¹ to 15×10¹¹, 15×10¹¹ to 20×10¹¹, 20×10¹¹ to 25×10¹¹, 25×10¹¹ to 30×10¹¹, 30×10¹¹ to 50×10¹¹, or 50×10¹¹ to 100×10¹¹ genome copies/mL. In some cases, suitable doses can be about 5×10¹¹ to 10×10¹¹, 10×10¹¹ to 25×10¹¹, or 25×10¹¹ to 50×10¹¹ genome copies/mL. In some cases, suitable doses can be at least about 5×10¹², 6×10¹², 7×10¹², 8×10¹², 9×10¹², 10×10¹², 11×10¹², 15×10¹², 20×10¹², 25×10¹², 30×10¹² or 50×10¹² genome copies/mL. In some embodiments, suitable doses can be about 5×10¹² to 6×10¹², 6×10¹² to 7×1V, 7×10¹² to 8×10¹², 8×10¹² to 9×10¹², 9×10¹² to 10×10¹², 10×10¹² to 11×10¹², 11×10¹² to 15×10¹², 15×10¹² to 20×10¹², 20×10¹² to 25×10¹², 25×10¹² to 30×10¹², 30×10¹² to 50×10¹², or 50×10¹² to 100×10¹² genome copies/mL. In some cases, suitable doses can be about 5×10¹² to 10×10¹², 10×10¹² to 25×10¹², or 25×10¹² to 50×10¹² genome copies/mL. In some cases, suitable doses can be at least about 5×10¹³, 6×10¹³, 7×10¹³, 8×10¹³, 9×10¹³, 10×10¹³, 11×10¹³, 15×10¹³, 20×10¹³, 25×10¹³, 30×10¹³ or 50×10¹³ genome copies/mL. In some embodiments, suitable doses can be about 5×10¹³ to 6×10¹³, 6×10¹³ to 7×10¹³, 7×10¹³ to 8×10¹³, 8×10¹³ to 9×10¹³, 9×10¹³ to 10×10¹³, 10×10¹³ to 11×10¹³, 11×10¹³ to 15×10¹³, 15×10¹³ to 20×10¹³, 20×10¹³ to 25×10¹³, 25×10¹³ to 30×10¹³, 30×10¹³ to 50×10¹³, or 50×10¹³ to 100×10¹³ genome copies/mL. In some cases, suitable doses can be about 5×10¹³ to 10×10¹³, 10×10¹³ to 25×10¹³, or 25×10¹³ to 50×10¹³ genome copies/mL. In some cases, suitable doses can be at least about 5×10¹³, 6×10¹³, 7×10¹³, 8×10¹³, 9×10¹³, 10×10¹³, 11×10¹³, 15×10¹³, 20×10¹³, 25×10¹³, 30×10¹³ or 50×10¹³ genome copies/mL. In some embodiments, suitable doses can be about 5×10¹³ to 6×10¹³, 6×10¹³ to 7×10¹³, 7×10¹³ to 8×10¹³, 8×10¹³ to 9×10¹³, 9×10¹³ to 10×10¹³, 10×10¹³ to 11×10¹³, 11×10¹³ to 15×10¹³, 15×10¹³ to 20×10¹³, 20×10¹³ to 25×10¹³, 25×10¹³ to 30×10¹³, 30×10¹³ to 50×10¹³, or 50×10¹³ to 100×10¹³ genome copies/mL. In some cases, suitable doses can be about 5×10¹³ to 10×10¹³, 10×10¹³ to 25×10¹³, or 25×10¹³ to 50×10¹³ genome copies/mL.

In some cases, the dose of virus particles administered to the individual can be any at least about 1×10⁷ to about 1×10¹³ genome copies/kg body weight. In some embodiments, the dose of virus particles administered to the individual can be 1×10⁷, 2×10⁷, 3×10⁷, 4×10⁷, 5×10⁷, 6×10⁷, 7×10⁷, 8×10⁷, or 9×10⁷ genome copies/kg body weight. In some embodiments, the dose of virus particles administered to the individual can be 1×10⁸, 2×10⁸, 3×10⁸, 4×10⁸, 5×10⁸, 6×10⁸, 7×10⁸, 8×10⁸, or 9×10⁸ genome copies/kg body weight. In some embodiments, the dose of virus particles administered to the individual can be 1×10⁹, 2×10⁹, 3×10⁹, 4×10⁹, 5×10⁹, 6×10⁹, 7×10⁹, 8×10⁹, or 9×10⁹ genome copies/kg body weight. In some embodiments, the dose of virus particles administered to the individual can be 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ genome copies/kg body weight. In some embodiments, the dose of virus particles administered to the individual can be 1×10¹¹, 2×10¹¹, 3×10¹¹, 4×10¹¹, 5×10¹¹, 6×10¹¹, 7×10¹¹, 8×10¹¹, or 9×10¹¹ genome copies/kg body weight. In some embodiments, the dose of virus particles administered to the individual can be 1×10¹², 2×10¹², 3×10¹², 4×10¹², 5×10¹², 6×10¹², 7×10¹², 8×10¹², or 9×10¹² genome copies/kg body weight. In some embodiments, the dose of virus particles administered to the individual can be 1×10¹³, 2×10¹³, 3×10¹³, 4×10¹³, 5×10¹³, 6×10¹³, 7×10¹³, 8×10¹³, or 9×10¹³ genome copies/kg body weight.

In some cases, compositions provided herein are utilized in conjunction, before, during, and/or after a secondary therapy. Secondary therapies can be associated with treatment of disease provided herein such as CNS and/or GI disease.

In some cases, as secondary therapy is utilized in Parkinson's Disease. Parkinson's Disease treatments can comprise medications, surgery, lifestyle change, or physical therapy. Medications can increase or substitute for dopamine. For example, levodopa can be a precursor to dopamine. It can be taken together with carbidopa, which can protect levodopa from early conversion to dopamine outside the brain. Carbidopa-levodopa can be taken orally or infused directly to the small intestine. Dopamine agonists, such as pramipexole, ropinirole, rotigotine, and apomorphine, mimic dopamine effects. Dopamine agonists can be taken orally or injected. Monoamine oxidase B inhibitors, such as selegiline, rasagiline, and safinamide, can inhibit the breakdown of brain dopamine. Catechol O-methyltransferase (COMT) inhibitors, such as entacapone and tolcapone, can prolong the effect of levodopa therapy by inhibiting the breakdown of dopamine. Anticholinergic medications, such as benztropine and trihexyphenidyl, and amantadine can also be used. Probiotic treatment, such as Bacillus subtilis, can be used to treat Parkinson's Disease patients. Surgical procedures can comprise deep brain stimulation, such as open pop-up dialog box Deep brain stimulation. Electrodes can be implanted into the brain and connected to a generator that can send electrical pulses to the brain to reduce Parkinson's Disease symptoms. In some cases, a secondary therapy comprises deep brain stimulation.

In some cases, as secondary therapy is utilized in Crohn's Disease. Crohn's Disease treatments can comprise medications, nutrition therapy, or surgery. Medications can comprise anti-inflammatory drugs, immune system suppressors, antibiotics, or others. Anti-inflammatory drugs can comprise corticosteroids and 5-aminosalicylates. Corticosteroids can comprise prednisone and budesonide (Entocort EC). 5-aminosalicylates can comprise sulfasalazine or mesalamine. Immune system suppressors can comprise azathioprine, mercaptopurine, infliximab, adalimumab, certolizumab pegol, methotrexate, natalizumab, vedolizumab, or ustekinumab. Antibiotics can comprise ciprofloxacin or metronidazole. Medications can also comprise anti-diarrheals, pain relievers, iron supplements, vitamin B-12 supplements, calcium supplements, or vitamin D supplements. Anti-diarrheal can comprise fiber supplements or loperamide. Fiber supplements can comprise psyllium powder or methylcellulose. Pain relievers can comprise acetaminophen. Pain relievers may not comprise ibuprofen or naproxen sodium. Nutrition therapy can comprise enteral nutrition or parenteral nutrition. Nutrition therapy can be combined with medications mentioned herein. Surgery can comprise the removal of a damaged portion of a digestive tract and reconnection of healthy sections. Surgery can comprise closure of fistulas or drainage abscesses.

Secondary therapies can be administered at any suitable dose. In some cases, a dose comprises: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78 79 80, 81, 82, 83, 84, 85, 86, 87, 88, 89, 90, 95, 100, 110, 120, 130, 140, 150, 160, 170, 180, 190, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or up to about 1000 mg/m² of a subject. These dosages can be administered daily, weekly, monthly, or yearly. These dosages can be administered once or multiple times.

In some cases, a pharmaceutical composition provided herein, or a secondary therapy can be administered either alone or together with a pharmaceutically acceptable carrier or excipient, by any routes, and such administration can be carried out in both single and multiple dosages. More particularly, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes.

Administration or application of a composition disclosed herein can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days. Administration or application of a composition disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. Administration can be performed repeatedly over a lifetime of a subject, such as once a month or once a year for the lifetime of a subject. Administration can be performed repeatedly over a substantial portion of a subject's life, such as once a month or once a year for at least about 1 year, 5 years, 10 years, 15 years, 20 years, 25 years, 30 years, or more.

Administration or application of composition disclosed herein can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration or application of composition disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, a composition can be administered/applied as a single dose or as divided doses. In some cases, the compositions described herein can be administered at a first time point and a second time point. In some cases, a composition can be administered such that a first administration is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

In some embodiments, compositions disclosed herein can be in unit dose forms or multiple-dose forms. For example, a pharmaceutical composition described herein can be in unit dose form. Unit dose forms, as used herein, refer to physically discrete units suitable for administration to human or non-human subjects (e.g., pets, livestock, non-human primates, and the like) and packaged individually. Each unit dose can contain a predetermined quantity of an active ingredient(s) that can be sufficient to produce the desired therapeutic effect in association with pharmaceutical carriers, diluents, excipients, or any combination thereof. Examples of unit dose forms can include, ampules, syringes, and individually packaged tablets and capsules. In some instances, a unit dose form can be comprised in a food, for example, a chocolate. In some instances, unit-dosage forms can be administered in fractions or multiples thereof. A multiple-dose form can be a plurality of identical unit dose forms packaged in a single container, which can be administered in segregated a unit dose form. Examples of a multiple-dose form can include vials, bottles of tablets or capsules, bottles of gummies, or bottles of pints or gallons. In some instances, a multiple-dose form can comprise different pharmaceutically active agents. In some embodiments, a unit dose form can be a serving. In some cases, a multiple-dose form can have more than about: 1, 2, 3, 4, 5, 6, 7, 8, 9, 10, 15, 20, 30, 40, 50, 60, 70, 80, 90, 100 or 200 servings. In some embodiments, a multiple-dose form can have less than about: 1, 2, 3, 4, 5, 6, 7, 8, 9, 10, 15, 20, 30, 40, 50, 60, 70, 80, 90, 100 or 200 servings. In some instances, a multiple-dose form can have from about: 1 serving to about 200 servings, 1 serving to about 20 servings, 5 servings to about 50 servings, 10 servings to about 100 servings, or about 30 servings to about 150 servings.

A composition described herein can compromise an excipient. An excipient can comprise a cryo-preservative, such as DMSO, glycerol, polyvinylpyrrolidone (PVP), or any combination thereof. An excipient can comprise a cryo-preservative, such as a sucrose, a trehalose, a starch, a salt of any of these, a derivative of any of these, or any combination thereof. An excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a triglyceride, an alcohol, or any combination thereof. An excipient can comprise sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. An excipient can be an excipient described in the Handbook of Pharmaceutical Excipients, American Pharmaceutical Association (1986).

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent. In some cases, an excipient can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts, or combinations thereof can be used in a pharmaceutical formulation.

An excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitors.

A pharmaceutical formulation can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

The binders that can be used in a pharmaceutical formulation can be selected from starches such as potato starch, corn starch, wheat starch; sugars such as sucrose, glucose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol, water, or a combination thereof.

A pharmaceutical formulation can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical formulation can be selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some cases, a pharmaceutical formulation can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isomorphous silicate, and microcrystalline cellulose as high HLB emulsifier surfactants.

In some embodiments, a pharmaceutical formulation can comprise a disintegrant as an excipient. In some instances, a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments, a disintegrant can be an effervescent disintegrant. Non-limiting examples of suitable effervescent disintegrants can include sodium bicarbonate in combination with citric acid, and sodium bicarbonate in combination with tartaric acid.

A pharmaceutical composition can comprise a diluent. Non-limiting examples of diluents can include water, glycerol, methanol, ethanol, and other similar biocompatible diluents. In some cases, a diluent can be an aqueous acid such as acetic acid, citric acid, maleic acid, hydrochloric acid, phosphoric acid, nitric acid, sulfuric acid, or similar. In other cases, a diluent can be selected from a group comprising alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, choline, lactose, maltose, mannitol, simethicone, sorbitol, starch, pregelatinized starch, talc, xylitol and/or anhydrates, hydrates and/or pharmaceutically acceptable derivatives thereof or combinations thereof.

A pharmaceutical composition can comprise a carrier. A carrier can be a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers can also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), which can be present singly or in combination, comprising alone or in combination 1-99.99% by weight or volume. Exemplary protein excipients include serum albumins such as human serum albumin (HSA), recombinant human albumin (rHA), gelatin, casein, and the like. Representative amino acid components, antibody components, or both, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients can be also intended within the scope of this technology, examples of which include but can be not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), and myoinositol.

Treating or treatments can comprise obtaining a desired pharmacologic effect, physiologic effect, or any combination thereof. In some instances, a treatment can reverse an adverse effect attributable to the disease or condition. In some cases, the treatment can stabilize the disease or condition. In some cases, the treatment can delay progression of the disease or condition. In some instances, the treatment can cause regression of the disease or condition. In some instances, a treatment can at least partially prevent the occurrence of the disease, condition, or a symptom of any of these. In some embodiments, a treatment's effect can be measured. In some cases, measurements can be compared before and after administration of the composition. For example, a subject can have medical images prior to treatment compared to images after treatment to show cancer regression. In some instances, a subject can have an improved blood test result after treatment compared to a blood test before treatment. In some instances, measurements can be compared to a standard.

### Kits

Any of the compositions described herein may be comprised in a kit. In a non-limiting example, a vector, a polynucleotide, a peptide, reagents to generate polynucleotides provided herein, and any combination thereof may be comprised in a kit. In some cases, kit components are provided in suitable container means.

Kits may comprise a suitably aliquoted composition. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third, or other additional container into which the additional components may be separately placed. However, various combinations of components may be comprised in a vial. The kits also will typically include a means for containing the components in close confinement for commercial sale. Such containers may include injection or blow-molded plastic containers into which the desired vials are retained.

However, the components of the kit may be provided as dried powder(s). When reagents and/or components are provided as a dry powder, the powder can be reconstituted by the addition of a suitable solvent. It is envisioned that the solvent may also be provided in another container means.

In some embodiments, a kit can comprise an engineered polynucleotide as disclosed herein (e.g., engineered guide RNA), a precursor engineered polynucleotide (e.g., a precursor engineered guide RNA), a vector comprising the engineered polynucleotide (e.g., engineered guide RNA or the precursor engineered guide RNA), or a nucleic acid of the engineered polynucleotide (e.g., engineered guide RNA or the precursor engineered guide RNA), or a pharmaceutical composition and a container. In some instances, a container can be plastic, glass, metal, or any combination thereof.

In some instances, a packaged product comprising a composition described herein can be properly labeled. In some instances, the pharmaceutical composition described herein can be manufactured according to good manufacturing practice (cGMP) and labeling regulations. In some cases, a pharmaceutical composition disclosed herein can be aseptic.

While preferred embodiments have been shown and described herein, it will be obvious to those skilled in the art that such embodiments are provided by way of example only. Numerous variations, changes, and substitutions will now occur to those skilled in the art. It should be understood that various alternatives to the embodiments described herein may be employed. It is intended that the following claims define the scope and that methods and structures within the scope of these claims and their equivalents be covered herein.

### Numbered Embodiments #1

Embodiment 1. An engineered polynucleotide that comprises a targeting sequence that is at least partially complementary to a region of a target RNA, wherein the region of the target RNA: (a) at least partially encodes for: a Leucine-rich repeat kinase 2 (LRRK2) polypeptide, an alpha-synuclein (SNCA) polypeptide, a glucosylceramidase beta (GBA) polypeptide, a PTEN-induced kinase 1 (PINK1) polypeptide, or a Tau polypeptide; (b) comprises a sequence that is proximal to (a); or (c) comprises (a) and (b), wherein the engineered polynucleotide is configured upon binding to the region of the target RNA, in association with the target RNA, to form a structural feature which at least in part recruits an RNA editing entity, wherein the RNA editing entity, when associated with the engineered polynucleotide and the target RNA, facilitates: an editing of a base of a nucleotide of a polynucleotide of the region of the target RNA, a modulation of the expression of the LRRK2 polypeptide, the SNCA polypeptide, the GBA polypeptide, the PINK1 polypeptide, the Tau polypeptide; or a combination thereof.

Embodiment 2. The engineered polynucleotide of embodiment 1, comprising (b), wherein the sequence that is proximal to the region of the target RNA at least partially encoding the LRRK2 polypeptide, the SNCA polypeptide, the GBA polypeptide, the PINK1 polypeptide, or the Tau polypeptide comprises at least a portion of a three prime untranslated region (3′ UTR).

Embodiment 3. The engineered polynucleotide of embodiment 1, comprising (b), wherein the sequence that is proximal to the region of the target RNA at least partially encoding the LRRK2 polypeptide, the SNCA polypeptide, the GBA polypeptide, the PINK1 polypeptide, or the Tau polypeptide comprises at least a portion of a five prime untranslated region (5′ UTR).

Embodiment 4. The engineered polynucleotide of embodiment 3, wherein the editing of the base of the 5′UTR results in at least partially regulating gene translation of the LRRK2 polypeptide, the SNCA polypeptide, the GBA polypeptide, the PINK1 polypeptide, or the Tau polypeptide.

Embodiment 5. The engineered polynucleotide of embodiment 3, wherein the editing of the base of the nucleotide of the polynucleotide of the region of the 5′UTR results in facilitating regulating mRNA translation of: the LRRK2 polypeptide, the SNCA polypeptide, the GBA polypeptide, the PINK1 polypeptide, or the Tau polypeptide.

Embodiment 6. The engineered polynucleotide of any one of embodiments 1-5, wherein the region of the target RNA at least partially encodes the LRRK2 polypeptide.

Embodiment 7. The engineered polynucleotide of embodiment 6, wherein the region of the target RNA that at least partially encodes the LRRK2 polypeptide comprises at least a portion of: a poly(A) tail, a microRNA response element (MRE), AU-rich element (ARE), hnRNP binding sites or any combination thereof.

Embodiment 8. The engineered polynucleotide of any one of embodiments 6-7, wherein the engineered polynucleotide is configured to modulate expression of the LRRK2 polypeptide.

Embodiment 9. The engineered polynucleotide of any one of embodiments 1-5, wherein the region of the target RNA at least partially encodes the SNCA polypeptide, and wherein the engineered polynucleotide is configured to modulate expression of the SNCA polypeptide.

Embodiment 10. The engineered polynucleotide of any one of embodiments 1-5, wherein the region of the target RNA at least partially encodes the GBA polypeptide, and wherein the engineered polynucleotide is configured to modulate expression of the GBA polypeptide.

Embodiment 11. The engineered polynucleotide of any one of embodiments 1-5, wherein the region of the target RNA at least partially encodes the PINK1 polypeptide, and wherein the engineered polynucleotide is configured to modulate expression of the PINK1 polypeptide.

Embodiment 12. The engineered polynucleotide of any one of embodiments 1-5, wherein the region of the target RNA at least partially encodes the Tau polypeptide, and wherein the engineered polynucleotide is configured to modulate expression of the Tau polypeptide.

Embodiment 13. The engineered polynucleotide of any one of embodiments 1-12, wherein the targeting sequence is about: 40, 60, 80, 100, or 120 nucleotides in length.

Embodiment 14. The engineered polynucleotide of embodiment 13, wherein the targeting sequence is about 100 nucleotides in length.

Embodiment 15. The engineered polynucleotide of any one of embodiments 1-14, wherein the targeting sequence that is at least partially complementary to the region of the target RNA comprises at least one nucleotide that is not complementary to a nucleotide in the region of the target RNA.

Embodiment 16. The engineered polynucleotide of embodiment 15, wherein the at least one nucleotide that is not complementary is an adenosine (A), and wherein the A is comprised in an A/C mismatch.

Embodiment 17. The engineered polynucleotide of any one of embodiments 1-16, wherein the target RNA is selected from the group comprising: an mRNA, a pre-mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA.

Embodiment 18. The engineered polynucleotide of embodiment 17, wherein the target RNA is an mRNA.

Embodiment 19. The engineered polynucleotide of any one of embodiments 6-7, wherein the region of the target RNA comprises: a region at least partially encoding a repeat domain of the LRRK2 polypeptide, a Ras-of-complex (Roc) GTPase domain of the LRRK2 polypeptide, a kinase domain of the LRRK2 polypeptide, a WD40 domain of the LRRK2 polypeptide, and a C-terminal of Roc (COR) domain of the LRRK2 polypeptide.

Embodiment 20. The engineered polynucleotide of embodiment 19, wherein the region of the target RNA comprises the region at least partially encoding the kinase domain of the LRRK2 polypeptide.

Embodiment 21. The engineered polynucleotide of embodiment 18, wherein the region of the target RNA comprises a mutation as compared to an otherwise comparable region encoding a wildtype LRRK2 polypeptide.

Embodiment 22. The engineered polynucleotide of embodiment 21, wherein the mutation comprises a polymorphism.

Embodiment 23. The engineered polynucleotide of any one of embodiments 1-22, wherein the engineered polynucleotide further comprises an RNA editing entity recruiting domain that is capable of recruiting the RNA editing entity.

Embodiment 24. The engineered polynucleotide of embodiment 23, wherein the RNA editing entity recruiting domain is at least 1 to about 75 nucleotides in length.

Embodiment 25. The engineered polynucleotide of embodiment 24, wherein the RNA editing entity recruiting domain is at least 30-50 nucleotides in length.

Embodiment 26. The engineered polynucleotide of any one of embodiments 1-25, wherein the RNA editing entity comprises an adenosine deaminase acting on RNA (ADAR) polypeptide or biologically active fragment thereof or adenosine deaminases acting on tRNA (ADAT) polypeptide or biologically active fragment thereof.

Embodiment 27. The engineered polynucleotide of embodiment 26, comprising the ADAR polypeptide or biologically active fragment thereof, which comprises ADAR1 or ADAR2.

Embodiment 28. The engineered polynucleotide of any one of embodiments 23-25, wherein the RNA editing entity recruiting domain comprises a glutamate ionotropic receptor AMPA type subunit 2 (GluR2) sequence.

Embodiment 29. The engineered polynucleotide of embodiment 28, wherein the GluR2 sequence comprises at least about 80%, 85%, 90%, 95%, or 99% sequence identity to SEQ ID NO: 1.

Embodiment 30. The engineered polynucleotide of embodiment 29, wherein the GluR2 sequence comprises SEQ ID NO: 1.

Embodiment 31. The engineered polynucleotide of any one of embodiments 1-22, wherein the engineered polynucleotide lacks a recruiting domain.

Embodiment 32. The engineered polynucleotide of any one of embodiments 1-31, wherein the structural feature comprises: a bulge, a hairpin, an internal loop, a structured motif, and any combination thereof.

Embodiment 33. The engineered polynucleotide of embodiment 32, wherein the structural feature comprises the bulge.

Embodiment 34. The engineered polynucleotide of embodiment 33, wherein the bulge is an asymmetric bulge.

Embodiment 35. The engineered polynucleotide of embodiment 33, wherein the bulge is a symmetric bulge.

Embodiment 36. The engineered polynucleotide of any one of embodiments 33-35, wherein the bulge is from 1-29 nucleotides in length.

Embodiment 37. The engineered polynucleotide of embodiment 32, wherein the structural feature comprises the hairpin.

Embodiment 38. The engineered polynucleotide of embodiment 32, wherein the structural feature comprises the internal loop.

Embodiment 39. The engineered polynucleotide of embodiment 32, wherein the structural feature comprises the structured motif.

Embodiment 40. The engineered polynucleotide of embodiment 39, wherein the structured motif comprises at least two of: the bulge, the hairpin, and the internal loop.

Embodiment 41. The engineered polynucleotide of embodiment 40, wherein the structured motif comprises the bulge and the hairpin.

Embodiment 42. The engineered polynucleotide of embodiment 40, wherein the structured motif comprises the bulge and the internal loop.

Embodiment 43. The engineered polynucleotide of any one of embodiments 1-42, wherein the engineered polynucleotide comprises a backbone that comprises a plurality of sugar and phosphate moieties covalently linked together, and wherein the backbone comprises a 5′ reducing hydroxyl, a 3′ reducing hydroxyl, or both.

Embodiment 44. The engineered polynucleotide of embodiment 43, wherein each of the 5′ reducing hydroxyl in the backbone is linked to each of the 3′ reducing hydroxyl via a phosphodiester bond.

Embodiment 45. The engineered polynucleotide of any one of embodiments 1-42, wherein the engineered polynucleotide comprises a backbone that comprises a plurality of sugar and phosphate moieties covalently linked together, and wherein the backbone lacks a 5′ reducing hydroxyl, a 3′ reducing hydroxyl, or both.

Embodiment 46. The engineered polynucleotide of any one of embodiments 1-40, wherein when the engineered polynucleotide associates with the region of the target RNA, the association comprises hybridized polynucleotide strands.

Embodiment 47. The engineered polynucleotide of embodiment 41, wherein the hybridized polynucleotide strands at least in part form a duplex.

Embodiment 48. The engineered polynucleotide of any one of embodiments 6-8, wherein the targeting sequence is capable of at least partially binding to a RNA at least partially encoding a LRRK2 polypeptide sequence that comprises at least 60%, 70%, 80%, 85%, 90%, 95%, 97%, or 99% sequence identity with at least a portion of SEQ ID NO: 73 or SEQ ID NO: 74 as determined by BLAST.

Embodiment 49. The engineered polynucleotide of embodiment 48, wherein the engineered polynucleotide comprises at least 60%, 70%, 80%, 85%, 90%, 95%, 97%, or 99% sequence identity with at least a portion of any one of: SEQ ID NO: 66-SEQ ID NO: 72.

Embodiment 50. The engineered polynucleotide of any one of embodiments 1-49, wherein the engineered polynucleotide further comprises a chemical modification.

Embodiment 51. The engineered polynucleotide of any one of embodiments 1-50, wherein the engineered polynucleotide comprises RNA, DNA, or both.

Embodiment 52. The engineered polynucleotide of embodiment 51, wherein the engineered polynucleotide comprises the RNA.

Embodiment 53. An engineered polynucleotide that comprises a targeting sequence that at least partially hybridizes to a region of a target RNA, wherein the region of the target RNA:
(a) at least partially encodes for a polypeptide selected from the group consisting of: an alpha-synuclein (SNCA), glucosylceramidase beta (GBA), PTEN-induced kinase 1 (PINK1), and Tau;
(b) comprises a sequence that is proximal to (a); or
(c) comprises (a) and (b); wherein the engineered polynucleotide is configured to: facilitate an editing of a base of a nucleotide of a polynucleotide of the region of the target RNA by an RNA editing entity; facilitate a modulation of the expression of the SNCA, the GBA, the PINK1, the Tau; or a combination thereof.

Embodiment 54. The engineered polynucleotide of embodiment 53, comprising the modulation of the expression of the SNCA, the GBA, the PINK1, or the Tau, wherein the modulation results in reduced expression of a polypeptide that codes for the SNCA, the GBA, the PINK1, or the Tau.

Embodiment 55. A vector that comprises: (a) the engineered polynucleotide of any one of embodiments 1-52; (b) the engineered polynucleotide of any one of embodiments 53-54; or (c) both (a) and (b).

Embodiment 56. The vector of embodiment 55, wherein the vector is a viral vector.

Embodiment 57. The vector of embodiment 56, wherein the viral vector is an AAV vector, and wherein the AAV vector is from an adeno-associated virus having a serotype selected from AAV1, AAV2, AAV3, AAV4, AAV5, AAV6, AAV7, AAV8, AAV9, AAV10, or AAV 11.

Embodiment 58. The vector of embodiment 57, wherein the AAV vector is a recombinant AAV (rAAV) vector, a hybrid AAV vector, a chimeric AAV vector, a self-complementary AAV (scAAV) vector, a single-stranded AAV or any combination thereof.

Embodiment 59. The vector of any one of embodiments 57-58, wherein the AAV vector comprises a genome comprising a replication gene and inverted terminal repeats from a first AAV serotype and a capsid protein from a second AAV serotype.

Embodiment 60. The vector of any one of embodiments 57-59, wherein the AAV vector is an AAV 2/5 vector, an AAV 2/6 vector, an AAV 2/7 vector, an AAV2/8 vector, or an AAV 2/9 vector.

Embodiment 61. The vector of any one of embodiments 59-60, wherein the inverted terminal repeats comprise a 5′ inverted terminal repeat, a 3′ inverted terminal repeat, and a mutated inverted terminal repeat.

Embodiment 62. The vector of embodiment 61, wherein the mutated inverted terminal repeat lacks a terminal resolution site.

Embodiment 63. A pharmaceutical composition in unit dose form that comprises: (a) the engineered polynucleotide of any one of embodiments 1-52; (b) the engineered polynucleotide of any one of embodiments 53-54, the vector of any one of embodiments 55-62, or any combination thereof; and (b) a pharmaceutically acceptable excipient, diluent, or carrier.

Embodiment 64. A method of making a pharmaceutical composition comprising admixing the engineered polynucleotide of any one of embodiment 1-54 with a pharmaceutically acceptable excipient, diluent, or carrier.

Embodiment 65. An isolated cell comprising the engineered polynucleotide of any one of embodiments 1-54, the vector of any one of embodiments 55-62, or both.

Embodiment 66. A kit comprising the engineered polynucleotide of any one of embodiments 1-54, the vector of any one of embodiments 55-62, or both in a container.

Embodiment 67. A method of making a kit comprising inserting the engineered polynucleotide of any one of embodiments 1-54, the vector of any one of embodiments 55-62, or both in a container.

Embodiment 68. A method of treating or preventing a disease or condition in a subject in need thereof, the method comprising administering to a subject in need thereof: (a) the vector of any one of embodiments 55-62; (b) the pharmaceutical composition of embodiment 63; or (c) (a) and (b).

Embodiment 69. A method of treating or preventing a disease or condition in a subject in need thereof, the method comprising administering to a subject in need thereof: a vector comprising or encoding an engineered polynucleotide, wherein the engineered polynucleotide comprises a targeting sequence that at least partially hybridizes to a region of a target RNA, wherein the region of the target RNA: (a) at least partially encodes for a Leucine-rich repeat kinase 2 (LRRK2) polypeptide; (b) comprises a sequence that is proximal to (a); or (c) comprises (a) and (b), and wherein the engineered polynucleotide is configured to facilitate an editing of a base of a nucleotide of a polynucleotide of the region of the target RNA by an RNA editing entity, modulate expression of the LRRK2 polypeptide, or any combination thereof; thereby treating or preventing the disease or condition in the subject in need thereof.

Embodiment 70. The method of embodiment 69, wherein the region of the target RNA comprises the sequence that is proximal to (a).

Embodiment 71. The method of embodiment 70, wherein the sequence that is proximal to (a) comprises at least a portion of a three prime untranslated region (3′ UTR).

Embodiment 72. The method of embodiment 70, wherein the sequence that is proximal to (a) comprises at least a portion of a five prime untranslated region (5′ UTR).

Embodiment 73. The method of embodiment 72, wherein the editing of the base of the 5′UTR results in at least partially regulating gene translation of the LRRK2 polypeptide.

Embodiment 74. The method of embodiment 73, wherein the editing of the base of the nucleotide of the polynucleotide of the region of the 5′UTR results in facilitating regulating mRNA translation of the LRRK2 polypeptide.

Embodiment 75. The method of any one of embodiments 69-74, wherein the sequence that is proximal to (a) comprises at least a portion of: a poly(A) tail, a microRNA response element (MRE), AU-rich element (ARE), hnRNP binding sites, or any combination thereof.

Embodiment 76. The method of any one of embodiments 69-75, wherein the engineered polynucleotide is configured to modulate expression of the LRRK2 polypeptide.

Embodiment 77. The method of any one of embodiments 69-76, wherein the region of the target RNA at least partially encodes for the LRRK2 polypeptide.

Embodiment 78. The method of any one of embodiments 69-77, wherein the engineered polynucleotide is configured to facilitate the editing of the base of the nucleotide of the polynucleotide of the region of the RNA by the RNA editing entity.

Embodiment 79. The method of any one of embodiments 69-78, wherein the targeting sequence comprises at least one nucleotide that is not complementary to a nucleotide in the region of the target RNA.

Embodiment 80. The method of embodiment 79, wherein the at least one nucleotide that is not complementary is an adenosine (A), and wherein the A is comprised in an A/C mismatch.

Embodiment 81. The method of any one of embodiments 69-80, wherein the administering comprises administering a therapeutically effective amount of the vector.

Embodiment 82. The method of any one of embodiments 69-81, wherein the administering at least partially treats or prevents at least one symptom of the disease or the condition in the subject in need thereof.

Embodiment 83. The method of any one of embodiments 69-82, wherein the target RNA is selected from the group comprising: a mRNA, a tRNA, a lncRNA, a lincRNA, a miRNA, a rRNA, a snRNA, a microRNA, a siRNA, a piRNA, a snoRNA, a snRNA, a exRNA, a scaRNA, a YRNA, and a hnRNA.

Embodiment 84. The method of embodiment 83, wherein the target RNA is the mRNA.

Embodiment 85. The method of any one of embodiments 69-84, wherein the region of the target RNA comprises: a region at least partially encoding a repeat domain of the LRRK2 polypeptide, Ras-of-complex (Roc) GTPase domain of the LRRK2 polypeptide, a kinase domain of the LRRK2 polypeptide, a WD40 domain of the LRRK2 polypeptide, and a C-terminal of Roc (COR) domain of the LRRK2 polypeptide.

Embodiment 86. The method of embodiment 85, wherein the region of the target RNA at least partially encodes the kinase domain of the LRRK2 polypeptide.

Embodiment 87. The method of embodiment 86, wherein the kinase domain of the LRRK2 polypeptide comprises a mutation relative to a wild type LRRK2 polypeptide.

Embodiment 88. The method of any one of embodiments 69-87, wherein the region of the target RNA comprises a nucleotide mutation as compared to an otherwise comparable region encoding a wildtype LRRK2 polypeptide.

Embodiment 89. The method of any one of embodiments 69-88, wherein the engineered polynucleotide is capable of recruiting an RNA editing entity.

Embodiment 90. The method of embodiment 89, wherein the engineered polynucleotide lacks an RNA editing entity recruiting domain.

Embodiment 91. The method of embodiment 89, wherein the engineered polynucleotide further comprises an RNA editing entity recruiting domain, at least a portion of which is capable of at least transiently associating with the RNA editing entity.

Embodiment 92. The method of embodiment 91, wherein the RNA editing entity recruiting domain recruits the RNA editing entity.

Embodiment 93. The method of any one of embodiments 91-92, wherein the RNA editing entity recruiting domain is at least 1 to about 75 nucleotides in length.

Embodiment 94. The method of embodiment 93, wherein the RNA editing entity recruiting domain is at least 30-50 nucleotides in length.

Embodiment 95. The method of any one of embodiments 69-94, wherein the RNA editing entity comprises an adenosine deaminase acting on RNA (ADAR) polypeptide or biologically active fragment thereof or adenosine deaminases acting on tRNA (ADAT) polypeptide or biologically active fragment thereof.

Embodiment 96. The method of embodiment 95, comprising the ADAR polypeptide or biologically active fragment thereof, which comprises ADAR1 or ADAR2.

Embodiment 97. The method of any one of embodiments 91-96, wherein the RNA editing entity recruiting domain comprises a glutamate ionotropic receptor AMPA type subunit 2 (GluR2) sequence.

Embodiment 98. The method of embodiment 97, wherein the engineered polynucleotide comprises at least two GluR2 sequences.

Embodiment 99. The method of any one of embodiments 97-98, wherein the GluR2 sequence comprises at least about 80%, 85%, 90%, 95%, or 99% sequence identity to SEQ ID NO: 1.

Embodiment 100. The method of embodiment 99, wherein the GluR2 sequence comprises SEQ ID NO: 1.

Embodiment 101. The method of any one of embodiments 91-96, wherein the engineered polynucleotide lacks a GluR2 sequence.

Embodiment 102. The method of any one of embodiments 69-101, wherein the engineered polynucleotide further comprises a structural feature, and wherein the structural feature comprises: a bulge, a hairpin, an internal loop, a structured motif, and any combination thereof.

Embodiment 103. The method of embodiment 102, wherein the engineered polynucleotide comprises the bulge.

Embodiment 104. The method of embodiment 103, wherein the engineered polynucleotide comprises at least 2, 3, 4, or up to 5 bulges.

Embodiment 105. The method of embodiments 103-104, wherein the bulge is from 1-29 nucleotides in length.

Embodiment 106. The method of any one of embodiments 103-105, wherein the bulge is an asymmetric bulge.

Embodiment 107. The method of any one of embodiments 103-105, wherein the bulge is a symmetric bulge.

Embodiment 108. The method of embodiment 102, wherein the engineered polynucleotide comprises the hairpin.

Embodiment 109. The method of embodiment 102, wherein the engineered polynucleotide comprises the internal loop.

Embodiment 110. The method of embodiment 109, wherein the internal loop is an asymmetric loop.

Embodiment 111. The method of embodiment 109, wherein the internal loop is a symmetric loop.

Embodiment 112. The method of embodiment 102, wherein the engineered polynucleotide comprises the structured motif.

Embodiment 113. The method of embodiment 112, wherein the structured motif comprises at least two of: the bulge, the hairpin, and the internal loop.

Embodiment 114. The method of embodiment 113, wherein the engineered polynucleotide comprises the bulge and the hairpin.

Embodiment 115. The method of embodiment 113, wherein the engineered polynucleotide comprises the bulge and the internal loop.

Embodiment 116. The method of any one of embodiments 69-115, wherein the engineered polynucleotide comprises a backbone that comprises a plurality of sugar and phosphate moieties covalently linked together, and wherein the backbone comprises a 5′ reducing hydroxyl, a 3′ reducing hydroxyl, or both.

Embodiment 117. The method of embodiment 116, wherein each of the 5′ reducing hydroxyl in the backbone is linked to each of the 3′ reducing hydroxyl via a phosphodiester bond

Embodiment 118. The method of any one of embodiments 69-115, wherein the engineered polynucleotide comprises a backbone that comprises a plurality of sugar and phosphate moieties covalently linked together, and wherein the backbone lacks a 5′ reducing hydroxyl, a 3′ reducing hydroxyl, or both.

Embodiment 119. The method of any one of embodiments 69-118, wherein at least a portion of the engineered polynucleotide hybridizes to the target RNA thereby forming two hybridized polynucleotide strands.

Embodiment 120. The method of embodiment 119, wherein the two hybridized polynucleotide strands form a duplex.

Embodiment 121. The method of any one of embodiments 69-120, wherein the targeting sequence is capable of at least partially binding to a sequence that comprises at least 60%, 70%, 80%, 85%, 90%, 95%, 97%, or 99% sequence identity with at least a portion of SEQ ID NO: 73 or SEQ ID NO: 74 as determined by BLAST.

Embodiment 122. The method of any one of embodiments 69-121, wherein the engineered polynucleotide comprises at least 60%, 70%, 80%, 85%, 90%, 95%, 97%, or 99% sequence identity with at least a portion of a sequence selected from: SEQ ID NO: 66-SEQ ID NO: 72.

Embodiment 123. The method of any one of embodiments 69-122, wherein the base of the nucleotide of the polynucleotide of the region of the engineered polynucleotide is modified, thereby generating an edited RNA, and wherein the edited RNA when translated generates a wildtype LRRK2 polypeptide.

Embodiment 124. The method of any one of embodiments 69-123, wherein the engineered polynucleotide is configured to revert a mutant residue selected from Table 3 to a corresponding wildtype residue of the LRRK2 polypeptide by the facilitating of the editing of the base of the nucleotide of the polynucleotide of the region of the target RNA that encodes the LRRK2 polypeptide.

Embodiment 125. The method of any one of embodiments 69-124, wherein the vector further comprises or encodes a second engineered polynucleotide.

Embodiment 126. The method of any one of embodiments 69-124, further comprising administering a second vector that comprises or encodes a second engineered polynucleotide.

Embodiment 127. The method of any one of embodiments 125-126, wherein the second engineered polynucleotide comprises a second targeting sequence that at least partially hybridizes to a region of a second target RNA.

Embodiment 128. The method of embodiment 127, wherein the second targeting sequence of the second engineered polynucleotide is at least partially complementary to the region of the second RNA.

Embodiment 129. The method of any one of embodiments 127-128, wherein the second target RNA at least partially encodes for a polypeptide that comprises: alpha-synuclein (SNCA), glucosylceramidase beta (GBA), PTEN-induced kinase 1 (PINK1), Tau, biologically active fragment of any of these, or any combination thereof.

Embodiment 130. The method of embodiment 129, wherein the second target RNA at least partially encodes for the SNCA polypeptide or biologically active fragment thereof.

Embodiment 131. The method of any one of embodiments 127-130, wherein the second engineered polynucleotide is configured to facilitate an editing of a base of a nucleotide of a polynucleotide of a region of the second target RNA by the RNA editing entity.

Embodiment 132. The method of embodiment 131, wherein the editing results in reduced expression of a polypeptide encoded by the second target RNA.

Embodiment 133. The method of any one of embodiments 69-132, wherein the vector is an AAV vector, and wherein the AAV vector is from an adeno-associated virus having a serotype selected from AAV1, AAV2, AAV3, AAV4, AAV5, AAV6, AAV7, AAV8, AAV9, AAV10, or AAV11.

Embodiment 134. The method of embodiment 133, wherein the AAV vector is a recombinant AAV (rAAV) vector, a hybrid AAV vector, a chimeric AAV vector, a self-complementary AAV (scAAV) vector, a single-stranded AAV or any combination thereof.

Embodiment 135. The method of any one of embodiments 133-134, wherein the AAV vector comprises a genome comprising a replication gene and inverted terminal repeats from a first AAV serotype and a capsid protein from a second AAV serotype.

Embodiment 136. The method of any one of embodiments 133-135, wherein the AAV vector is an AAV 2/5 vector, an AAV 2/6 vector, an AAV 2/7 vector, an AAV2/8 vector, or an AAV 2/9 vector.

Embodiment 137. The method of any one of embodiments 135-136, wherein the inverted terminal repeats comprise a 5′ inverted terminal repeat, a 3′ inverted terminal repeat, and a mutated inverted terminal repeat.

Embodiment 138. The method of embodiment 137, wherein the mutated inverted terminal repeat lacks a terminal resolution site.

Embodiment 139. The method of any one of embodiments 69-132, wherein the vector is a minicircle vector.

Embodiment 140. The method of any one of embodiments 69-132, wherein the vector is a DNA vector.

Embodiment 141. The method of any one of embodiments 69-140, wherein the vector comprises at least 60%, 70%, 80%, 85%, 90%, 95%, 97%, or 99% sequence identity with any one of SEQ ID NO: 78-80 as determined by BLAST.

Embodiment 142. The method of any one of embodiments 69-141, wherein the targeting portion comprises at least 60%, 70%, 80%, 85%, 90%, 95%, 97%, or 99% sequence identity with at least a portion of SEQ ID NO: 66-SEQ ID NO: 72 as determined by BLAST.

Embodiment 143. The method of embodiment 142, wherein the engineered polynucleotide comprises a sequence of SEQ ID NO: 66-SEQ ID NO: 72.

Embodiment 144. The method of any one of embodiments 69-143, wherein the disease or condition is of a central nervous system (CNS), gastrointestinal (GI) tract, or both.

Embodiment 145. The method of embodiment 144, wherein the disease is of both, and wherein the disease is Parkinson's Disease.

Embodiment 146. The method of embodiment 144, wherein the disease is of the GI tract, and wherein the disease is Crohn's disease.

Embodiment 147. The method of any one of embodiments 69-146, further comprising administering a secondary therapy.

Embodiment 148. The method of embodiment 147, wherein the secondary therapy is administered concurrent or sequential to the vector.

Embodiment 149. The method of embodiments 147-148, wherein the secondary therapy comprises at least one of a probiotic, a carbidopa, a levodopa, a MAO B inhibitor, a catechol O-methyltransferase (COMT) inhibitor, a anticholinergic, a amantadine, a deep brain stimulation, a salt of any of these, or any combination thereof.

Embodiment 150. The method of any one of embodiments 147-149, wherein the administering of the vector, the secondary therapy, or both are independently performed at least about: 1 time per day, 2 times per day, 3 times per day, 4 times per day, once a week, twice a week, 3 times a week, biweekly, bimonthly, monthly, or yearly.

Embodiment 151. The method of any one of embodiments 69-150, further comprising monitoring the disease or condition of the subject.

Embodiment 152. The method of any one of embodiments 69-151, wherein the vector is comprised in a pharmaceutical composition in unit dose form.

Embodiment 153. The method of any one of embodiments 69-152, wherein the subject is diagnosed with the disease or the condition prior to the administering.

Embodiment 154. The method of embodiment 153, wherein the diagnosing is via an in vitro assay.

Embodiment 155. The method of any one of embodiments 69-154, wherein the editing of the base of the nucleotide of the polynucleotide of the region of the target RNA comprises at least about 3%, 5%, 10%, 15%, or 20% editing as measured by sequencing.

Embodiment 156. The method of embodiment 155, wherein the second target RNA at least partially encodes for the SNCA polypeptide, and wherein the editing of the base of the nucleotide of the polynucleotide of the region of the target RNA by an ADAR polypeptide results in a modified polypeptide that comprises a change in a residue, as compared to an unmodified polypeptide encoded by the target RNA, that comprises: (a) an adenine to an inosine at a position corresponding to position 2019 of the LRRK2 polypeptide of SEQ ID NO: 15; (b) an adenine to an inosine at a position corresponding to position 30 or 53 of the SNCA polypeptide of SEQ ID NO: 34; or (c) a) and b).

### Numbered Embodiments #2

Embodiment 1. An engineered polynucleotide that comprises a targeting sequence that is at least partially complementary to a region of a target RNA, wherein the target RNA:
(a) encodes for: a Leucine-rich repeat kinase 2 (LRRK2) polypeptide, an alpha-synuclein (SNCA) polypeptide, a glucosylceramidase beta (GBA) polypeptide, a PTEN-induced kinase 1 (PINK1) polypeptide, or a Tau polypeptide;
(b) comprises a non-coding sequence; or
(c) comprises (a) and (b),
wherein the engineered polynucleotide is configured upon binding to the region of the target RNA, in association with the target RNA, to form a structural feature which recruits an RNA editing entity, wherein the RNA editing entity, when associated with the engineered polynucleotide and the region of the target RNA, facilitates: an editing of a base of a nucleotide in the region of the target RNA, a modulation of translation of the LRRK2 polypeptide, the SNCA polypeptide, the GBA polypeptide, the PINK1 polypeptide, the Tau polypeptide from the target RNA; or a combination thereof.

Embodiment 2. The engineered polynucleotide of embodiment 1, wherein the targeting sequence is about: 40, 45, 60, 80, 100, or 120 nucleotides in length.

Embodiment 3. The engineered polynucleotide of embodiment 1 or 2, wherein the targeting sequence is about 100 nucleotides in length.

Embodiment 4. The engineered polynucleotide of any one of embodiments 1-3, wherein the targeting sequence that is at least partially complementary to the region of the target RNA comprises at least one nucleotide that is not complementary to a nucleotide in the region of the target RNA.

Embodiment 5. The engineered polynucleotide of embodiment 4, wherein the at least one nucleotide that is not complementary is an adenosine (A) in the region of the target RNA, and wherein the A is comprised in an A/C mismatch.

Embodiment 6. The engineered polynucleotide of embodiment 4, wherein the at least one nucleotide that is not complementary is an adenosine (A) in the region of the target RNA, and wherein the A is comprised in an internal loop or bulge.

Embodiment 7. The engineered polynucleotide of any one of embodiments 4-6, wherein the A is the base of the nucleotide in the region of the target RNA for editing.

Embodiment 8. The engineered polynucleotide of any one of embodiments 1-7, wherein the target RNA is selected from the group comprising: an mRNA, a pre-mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA.

Embodiment 9. The engineered polynucleotide of any one of embodiments 1-8, wherein the target RNA is an mRNA.

Embodiment 10. The engineered polynucleotide of any one of embodiments 1-9, wherein the structural feature comprises: a bulge, a hairpin, an internal loop, and any combination thereof.

Embodiment 11. The engineered polynucleotide of any one of embodiments 1-10, wherein the structural feature comprises a bulge.

Embodiment 12. The engineered polynucleotide of embodiment 11, wherein the bulge is an asymmetric bulge.

Embodiment 13. The engineered polynucleotide of embodiment 11, wherein the bulge is a symmetric bulge.

Embodiment 14. The engineered polynucleotide of any one of embodiments 11-13, wherein the bulge is from 1-4 nucleotides in length.

Embodiment 15. The engineered polynucleotide of any one of embodiments 1-10, wherein the structural feature comprises a hairpin.

Embodiment 16. The engineered polynucleotide of any one of embodiments 1-10, wherein the structural feature comprises an internal loop.

Embodiment 17. The engineered polynucleotide of embodiment 16, wherein the internal loop is from 5-50 nucleotides in length on each side of the internal loop.

Embodiment 18. The engineered polynucleotide of embodiment 16 or 17, wherein the internal loop is 6 nucleotides in length on each side of the internal loop.

Embodiment 19. The engineered polynucleotide of any one of embodiments 1-18 comprising at least two internal loops.

Embodiment 20. The engineered polynucleotide of any one of embodiments 1-19 comprising two internal loops.

Embodiment 21. The engineered polynucleotide of embodiment 20, wherein the two internal loops are internal symmetrical loops.

Embodiment 22. The engineered polynucleotide of embodiments 20 or 21, wherein the two internal loops are internal symmetrical loops and each side of the two internal loop is 6 nucleotides in length.

Embodiment 23. The engineered polynucleotide of embodiment 16, wherein the internal loop is an asymmetrical internal loop.

Embodiment 24. The engineered polynucleotide of any one of embodiments 1-23 comprising a structured motif.

Embodiment 25. The engineered polynucleotide of embodiment 24, wherein the structured motif comprises at least two of: a bulge, a hairpin, and an internal loop.

Embodiment 26. The engineered polynucleotide of embodiment 25, wherein the structured motif comprises the bulge and the hairpin.

Embodiment 27. The engineered polynucleotide of embodiment 25, wherein the structured motif comprises the bulge and the internal loop.

Embodiment 28. The engineered polynucleotide of any one of embodiments 1-27, wherein the engineered polynucleotide lacks a recruiting domain.

Embodiment 29. The engineered polynucleotide of any one of embodiments 1-28, wherein the RNA editing entity comprises an adenosine deaminase acting on RNA (ADAR) polypeptide or biologically active fragment thereof or adenosine deaminases acting on tRNA (ADAT) polypeptide or biologically active fragment thereof.

Embodiment 30. The engineered polynucleotide of embodiment 29, wherein the ADAR polypeptide or biologically active fragment thereof comprises ADAR1 or ADAR2.

Embodiment 31. The engineered polynucleotide of any one of embodiments 1-30, wherein the engineered polynucleotide further comprises an RNA editing entity recruiting domain that is capable of recruiting the RNA editing entity.

Embodiment 32. The engineered polynucleotide of embodiment 31, wherein the RNA editing entity recruiting domain is at least 1 to about 75 nucleotides in length.

Embodiment 33. The engineered polynucleotide of embodiment 31 or 32, wherein the RNA editing entity recruiting domain is at least 30-50 nucleotides in length.

Embodiment 34. The engineered polynucleotide of any one of embodiments 31-33, wherein the RNA editing entity recruiting domain comprises a glutamate ionotropic receptor AMPA type subunit 2 (GluR2) sequence.

Embodiment 35. The engineered polynucleotide of embodiment 34, wherein the GluR2 sequence comprises at least about 80%, 85%, 90%, 95%, or 99% sequence identity to SEQ ID NO: 1.

Embodiment 36. The engineered polynucleotide of embodiment 34, wherein the GluR2 sequence comprises SEQ ID NO: 1.

Embodiment 37. The engineered polynucleotide of any one of embodiments 1-36, wherein the region is from 5 to 600 nucleotides in length of the target RNA, 40 to 400 nucleotides in length, or 80 to 120 nucleotides in length.

Embodiment 38. The engineered polynucleotide of any one of embodiments 1-37, wherein the region is from 50 to 200 nucleotides in length of the target RNA.

Embodiment 39. The engineered polynucleotide of any one of embodiments 1-38, wherein the region is about 100 nucleotides in length of the target RNA.

Embodiment 40. The engineered polynucleotide of any one of embodiments 1-39, wherein the region of the target RNA comprises at least 60%, 70%, 80%, 85%, 90%, 95%, 97%, 99%, or 100% sequence identity to SEQ ID NO: 73 or SEQ ID NO: 74.

Embodiment 41. The engineered polynucleotide of any one of embodiments 1-40, wherein the non-coding sequence comprises a three prime untranslated region (3′ UTR).

Embodiment 42. The engineered polynucleotide of any one of embodiments 1-41, wherein the non-coding sequence comprises a five prime untranslated region (5′ UTR).

Embodiment 43. The engineered polynucleotide of embodiment 42, wherein the editing of the base in the 5′UTR of the region of the target RNA results in at least partially regulating gene translation of the LRRK2 polypeptide, the SNCA polypeptide, the GBA polypeptide, the PINK1 polypeptide, or the Tau polypeptide.

Embodiment 44. The engineered polynucleotide of embodiment 42, wherein the editing of the base in the 5′UTR of the region of the target RNA results in facilitating regulation mRNA translation of: the LRRK2 polypeptide, the SNCA polypeptide, the GBA polypeptide, the PINK1 polypeptide, or the Tau polypeptide.

Embodiment 45. The engineered polynucleotide of any one of embodiments 1-44, wherein the target RNA encodes the LRRK2 polypeptide.

Embodiment 46. The engineered polynucleotide of embodiment 45, wherein the target RNA that encodes the LRRK2 polypeptide comprises at least a portion of: a poly(A) tail, a microRNA response element (MRE), AU-rich element (ARE), hnRNP binding sites or any combination thereof.

Embodiment 47. The engineered polynucleotide of embodiments 45 or 46, wherein the engineered polynucleotide is configured to modulate expression of the LRRK2 polypeptide.

Embodiment 48. The engineered polynucleotide of any one of embodiments 1-44, wherein target RNA encodes the SNCA polypeptide, and wherein the engineered polynucleotide is configured to modulate expression of the SNCA polypeptide.

Embodiment 49. The engineered polynucleotide of any one of embodiments 1-44, wherein the target RNA encodes the GBA polypeptide, and wherein the engineered polynucleotide is configured to modulate expression of the GBA polypeptide.

Embodiment 50. The engineered polynucleotide of any one of embodiments 1-44, wherein the target RNA encodes the PINK1 polypeptide, and wherein the engineered polynucleotide is configured to modulate expression of the PINK1 polypeptide.

Embodiment 51. The engineered polynucleotide of any one of embodiments 1-44, wherein the target RNA encodes the Tau polypeptide, and wherein the engineered polynucleotide is configured to modulate expression of the Tau polypeptide.

Embodiment 52. The engineered polynucleotide of any one of embodiments 45-47, wherein the target RNA encodes a repeat domain of the LRRK2 polypeptide, a Ras-of-complex (Roc) GTPase domain of the LRRK2 polypeptide, a kinase domain of the LRRK2 polypeptide, a WD40 domain of the LRRK2 polypeptide, or a C-terminal of Roc (COR) domain of the LRRK2 polypeptide.

Embodiment 53. The engineered polynucleotide of embodiment 52, wherein the target RNA encodes the kinase domain of the LRRK2 polypeptide.

Embodiment 54. The engineered polynucleotide of any one of embodiments 1-53, wherein the region of the target RNA comprises a mutation as compared to an otherwise comparable region encoding a wildtype polypeptide.

Embodiment 55. The engineered polynucleotide of any one of embodiments 1-54, wherein the region of the target RNA comprises a mutation as compared to an otherwise comparable region encoding a wildtype LRRK2 polypeptide.

Embodiment 56. The engineered polynucleotide of embodiments 54 or 55, wherein the mutation comprises a polymorphism.

Embodiment 57. The engineered polynucleotide of any one of embodiments 54-56, wherein the mutation is a G to A mutation.

Embodiment 58. The engineered polypeptide of any one of embodiments 1-47, 52, 53, or 55-57, wherein the target RNA comprises at least 80%, 90%, 95%, 97%, 98%, 99%, or 100% sequence identity to any one of SEQ ID NO: 5-SEQ ID NO: 14.

Embodiment 59. The engineered polypeptide of any one of embodiments 1-47, 52, 53, or 55-58, wherein the target RNA encodes a LRRK2 polypeptide comprising at least 80%, 90%, 95%, 97%, 98%, 99%, or 100% sequence identity to any one of SEQ ID NO: 15-SEQ ID NO: 24.

Embodiment 60. The engineered polynucleotide of any one of embodiments 1-47, 52, 53, or 55-59, wherein the target RNA encodes a LRRK2 polypeptide comprising a mutation corresponding a G2019S of SEQ ID NO: 15.

Embodiment 61. The engineered polynucleotide of embodiment 1-47, 52, 53, or 55-60, wherein the editing of the base is editing of an A corresponding to the 6055^{th} nucleotide in SEQ ID NO: 5.

Embodiment 62. The engineered polynucleotide of any one of embodiments 1-47, 52, 53, or 55-61, wherein the target RNA encodes a LRRK2 polypeptide comprising a mutation corresponding to a mutation of Table 3, or any combination of mutations of Table 3.

Embodiment 63. The engineered polypeptide of any one of embodiments 1-44, 48, 54, 56, or 57, wherein the target RNA comprises at least 80%, 90%, 95%, 97%, 98%, 99%, or 100% sequence identity to any one of SEQ ID NO: 25-SEQ ID NO: 33.

Embodiment 64. The engineered polypeptide of any one of embodiments 1-44, 48, 54, 56, 57, or 63, wherein the target RNA encodes a SCNA polypeptide comprising at least 80%, 90%, 95%, 97%, 98%, 99%, or 100% sequence identity to any one of SEQ ID NO: 34-SEQ ID NO: 36.

Embodiment 65. The engineered polynucleotide of any one of embodiments 1-44, 48, 54, 56, 57, 63, or 64, wherein the target RNA encodes a SNCA polypeptide comprising a mutation corresponding to a mutation of Table 6, or any combination of mutations of Table 6.

Embodiment 66. The engineered polynucleotide of any one of embodiments 1-44, 48, 54, 56, 57, or 63-65, wherein the editing of the base is the editing of Adenosine (A) of a translational initiation site of the target RNA that encodes a SNCA polypeptide.

Embodiment 67. The engineered polypeptide of any one of embodiments 1-44, 49, 54, 56, or 57, wherein the target RNA comprises at least 80%, 90%, 95%, 97%, 98%, 99%, or 100% sequence identity to any one of SEQ ID NO: 37-SEQ ID NO: 48.

Embodiment 68. The engineered polynucleotide of any one of embodiments 1-44, 49, 54, 56, 57, or 67, wherein the editing of the base is the editing of Adenosine (A) of a translational initiation site of the target RNA that encodes a Tau polypeptide.

Embodiment 69. The engineered polypeptide of any one of embodiments 1-44, 50, 54, 56, or 57, wherein the target RNA comprises at least 80%, 90%, 95%, 97%, 98%, 99%, or 100% sequence identity to SEQ ID NO: 49.

Embodiment 70. The engineered polynucleotide of any one of embodiments 1-44, 50, 54, 56, 57, or 69, wherein the editing of the base is the editing of Adenosine (A) of a translational initiation site of the target RNA that encodes a PINK1 polypeptide.

Embodiment 71. The engineered polypeptide of any one of embodiments 1-44, 50, 54, 56, or 57, wherein the target RNA comprises at least 80%, 90%, 95%, 97%, 98%, 99%, or 100% sequence identity to any one of SEQ ID NO: 50-SEQ ID NO: 54.

Embodiment 72. The engineered polynucleotide of any one of embodiments 1-44, 51, 54, 56, 57, or 71, wherein the editing of the base is the editing of Adenosine (A) of a translational initiation site of the target RNA that encodes a GBA polypeptide.

Embodiment 73. The engineered polynucleotide of any one of embodiments 1-47, 52, 53, or 55-62, wherein the engineered polynucleotide comprises at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182.

Embodiment 74. The engineered polynucleotide of any one of embodiments 1-44, 48, 54, 56, 57, or 63-66, wherein the engineered polynucleotide comprises at least 60%, 70%, 80%, 85%, 90%, 95%, 97%, 99%, or 100% sequence identity to any one of: SEQ ID NO: 183-SEQ ID NO: 192.

Embodiment 75. The engineered polynucleotide of any one of embodiments 1-74, wherein when the engineered polynucleotide associates with the region of the target RNA, the association comprises hybridized polynucleotide strands.

Embodiment 76. The engineered polynucleotide of embodiment 75, wherein the hybridized polynucleotide strands at least in part form a duplex.

Embodiment 77. The engineered polynucleotide of any one of embodiments 1-76, wherein the engineered polynucleotide further comprises a chemical modification.

Embodiment 78. The engineered polynucleotide of any one of embodiments 1-77, wherein the engineered polynucleotide comprises RNA, DNA, or both.

Embodiment 79. The engineered polynucleotide of embodiment 78, wherein the engineered polynucleotide comprises the RNA.

Embodiment 80. An engineered polynucleotide that comprises a targeting sequence that at least partially hybridizes to a region of a target RNA, wherein the target RNA:
(a) encodes for a polypeptide selected from the group consisting of: a Leucine-rich repeat kinase 2 (LRRK2), an alpha-synuclein (SNCA), glucosylceramidase beta (GBA), PTEN-induced kinase 1 (PINK1), and Tau;
(b) comprises a non-coding sequence; or
(c) comprises (a) and (b); wherein the engineered polynucleotide is configured to: facilitate an editing of a base of a nucleotide of the region of the target RNA by an RNA editing entity; facilitate a modulation of the expression of the LRRK2, SNCA, the GBA, the PINK1, the Tau; or a combination thereof.

Embodiment 81. A vector that comprises: (a) the engineered polynucleotide of any one of embodiments 1-79; (b) the engineered polynucleotide of embodiment 80; or (c) both (a) and (b).

Embodiment 82. The vector of embodiment 81, wherein the vector is a viral vector.

Embodiment 83. The vector of embodiment 82, wherein the viral vector is an AAV vector, and wherein the AAV vector is from an adeno-associated virus having a serotype selected from AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68.

Embodiment 84. The vector of embodiment 83, wherein the AAV vector is a recombinant AAV (rAAV) vector, a hybrid AAV vector, a chimeric AAV vector, a self-complementary AAV (scAAV) vector, a single-stranded AAV or any combination thereof.

Embodiment 85. The vector of any one of embodiments 83-84, wherein the AAV vector comprises a genome comprising a replication gene and inverted terminal repeats from a first AAV serotype and a capsid protein from a second AAV serotype.

Embodiment 86. The vector of any one of embodiments 83-85, wherein the AAV vector is an AAV 2/5 vector, an AAV 2/6 vector, an AAV 2/7 vector, an AAV2/8 vector, or an AAV 2/9 vector.

Embodiment 87. The vector of any one of embodiments 85-86, wherein the inverted terminal repeats comprise a 5′ inverted terminal repeat, a 3′ inverted terminal repeat, and a mutated inverted terminal repeat.

Embodiment 88. The vector of embodiment 87, wherein the mutated inverted terminal repeat lacks a terminal resolution site.

Embodiment 89. A pharmaceutical composition in unit dose form that comprises: (a) the engineered polynucleotide of any one of embodiments 1-79; (b) the engineered polynucleotide of embodiment 80, the vector of any one of embodiments 81-88, or any combination thereof; and (b) a pharmaceutically acceptable excipient, diluent, or carrier.

Embodiment 90. A method of making a pharmaceutical composition comprising admixing the engineered polynucleotide of any one of embodiment 1-80 with a pharmaceutically acceptable excipient, diluent, or carrier.

Embodiment 91. An isolated cell comprising the engineered polynucleotide of any one of embodiments 1-80, the vector of any one of embodiments 81-88, or both.

Embodiment 92. A kit comprising the engineered polynucleotide of any one of embodiments 1-80, the vector of any one of embodiments 81-88, or both in a container.

Embodiment 93. A method of making a kit comprising inserting the engineered polynucleotide of any one of embodiments 1-80, the vector of any one of embodiments 81-88, or both in a container.

Embodiment 94. A method of treating or preventing a disease or condition in a subject in need thereof, the method comprising administering to a subject in need thereof: (a) the vector of any one of embodiments 81-88; (b) the pharmaceutical composition of embodiment 89; or (c) (a) and (b).

Embodiment 95. The method of embodiment 94, wherein the administering comprises administering a therapeutically effective amount of the vector.

Embodiment 96. The method of embodiments 94 or 95, wherein the administering at least partially treats or prevents at least one symptom of the disease or the condition in the subject in need thereof.

Embodiment 97. The method of any one of embodiments 94-96, wherein the vector further comprises or encodes a second engineered polynucleotide.

Embodiment 98. The method of any one of embodiments 94-96, further comprising administering a second vector that comprises or encodes a second engineered polynucleotide.

Embodiment 99. The method of embodiment 97 or 98, wherein the second engineered polynucleotide comprises a second targeting sequence that at least partially hybridizes to a region of a second target RNA.

Embodiment 100. The method of embodiment 99, wherein the second targeting sequence of the second engineered polynucleotide is at least partially complementary to the region of the second target RNA.

Embodiment 101. The method of any one of embodiments of embodiments 99 or 100, wherein the second target RNA encodes for a polypeptide that comprises: alpha-synuclein (SNCA), glucosylceramidase beta (GBA), PTEN-induced kinase 1 (PINK1), Tau, biologically active fragment of any of these, or any combination thereof.

Embodiment 102. The method of embodiment 101, wherein the second target RNA encodes for the SNCA polypeptide or biologically active fragment thereof.

Embodiment 103. The method of any one of embodiments 97-102, wherein the second engineered polynucleotide is configured to facilitate an editing of a base of a nucleotide of a polynucleotide of a region of the second target RNA by the RNA editing entity.

Embodiment 104. The method of embodiment 103, wherein the editing results in reduced expression of a polypeptide encoded by the second target RNA.

Embodiment 105. The method of any one of embodiments 94-104, wherein the disease or condition is of a central nervous system (CNS), gastrointestinal (GI) tract, or both.

Embodiment 106. The method of embodiment 105, wherein the disease is of both, and wherein the disease is Parkinson's Disease.

Embodiment 107. The method of embodiment 105, wherein the disease is of the GI tract, and wherein the disease is Crohn's disease.

Embodiment 108. The method of any one of embodiments 94-107, further comprising administering a secondary therapy.

Embodiment 109. The method of embodiment 108, wherein the secondary therapy is administered concurrent or sequential to the vector.

Embodiment 110. The method of embodiments 108-109, wherein the secondary therapy comprises at least one of a probiotic, a carbidopa, a levodopa, a MAO B inhibitor, a catechol O-methyltransferase (COMT) inhibitor, a anticholinergic, a amantadine, a deep brain stimulation, a salt of any of these, or any combination thereof.

Embodiment 111. The method of any one of embodiments 108-110, wherein the administering of the vector, the secondary therapy, or both are independently performed at least about: 1 time per day, 2 times per day, 3 times per day, 4 times per day, once a week, twice a week, 3 times a week, biweekly, bimonthly, monthly, or yearly.

Embodiment 112. The method of any one of embodiments 94-111, further comprising monitoring the disease or condition of the subject.

Embodiment 113. The method of any one of embodiments 94-112, wherein the vector is comprised in a pharmaceutical composition in unit dose form.

Embodiment 114. The method of any one of embodiments 94-113, wherein the subject is diagnosed with the disease or the condition prior to the administering.

Embodiment 115. The method of embodiment 114, wherein the diagnosing is via an in vitro assay.

Embodiment 116. The method of any one of embodiments 94-115, wherein the editing of the base of the nucleotide of the polynucleotide of the region of the target RNA comprises at least about 3%, 5%, 10%, 15%, or 20% editing as measured by sequencing.

Embodiment 117. The method of embodiment 116, wherein the second target RNA encodes for the SNCA polypeptide, and wherein the editing of the base of the nucleotide of the polynucleotide of the region of the target RNA by an ADAR polypeptide results in a modified polypeptide that comprises a change in a residue, as compared to an unmodified polypeptide encoded by the target RNA, that comprises: (a) an adenine to an inosine at a position corresponding to position 2019 of the LRRK2 polypeptide of SEQ ID NO: 15; (b) an adenine to an inosine at a position corresponding to position 30 or 53 of the SNCA polypeptide of SEQ ID NO: 34; or (c) (a) and (b).

### EXAMPLES

### Example 1: Introduction of In Vitro Transcribed (IVT) Guide RNA (Exemplary Subject Engineered Polynucleotide)

gBlocks™ Gene Fragments were purchased from IDT and were used to generate IVT guide RNA, Table 10.

TABLE 10
guide RNA gBlocks ™
Formatting indicates various elements of each construct:
non-transcribed T7 promoter elements (lowercase); primer binding (underlined);
Recruiting sequences (GluR2) are italicized; ^{∧} denotes a nucleotide mismatch;
* denotes a nucleotide that will form a bulge when guide RNA hybridizes to target
region; # denotes a nucleotide that will form a loop when guide RNA hybridizes to
target region.

| SEQ ID | | |
|---|---|---|
| NO: | Guide RNA | Sequence |
| 55 | LRRK2_0.100.50 | atatgctaatacgactcactatagGTGCCCTCTGATGTTTTTATC |
| | | CCCATTCTACAGCAGTACTGAGCAATGC^{∧}CGTAGT |
| | | CAGCAATCTTTGCAATGATGGCAGCATTGGGATA |
| | | CAGTGTGAAAA |
| 56 | LRRK2_1.100.50 | atatgctaatacgactcactatagGTGCCCTCTGATGTTTTTATC |
| | | CCCATTCTACAGCAGTACTGAGCAATGC^{∧}CGTAGT |
| | | CAGCAATCTTTGCAATGATGGCAGCATTGGGATA |
| | | CAGTGTGAAAAGTGGAATAGTATAACAATATGCTAAA |
| | | TGTTGTTATAGTATCCCAC |
| 57 | LRRK2_2.100.50 | atatgctaatacgactcactatagGTGGAATAGTATAACAATATGC |
| | | TAAATGTTGTTATAGTATCCCACGTGCCCTCTGATGT |
| | | TTTTATCCCCATTCTACAGCAGTACTGAGCAATGC |
| | | ^{∧}CGTAGTCAGCAATCTTTGCAATGATGGCAGCATT |
| | | GGGATACAGTGTGAAAAGTGGAATAGTATAACAATA |
| | | TGCTAAATGTTGTTATAGTATCCCAC |
| 58 | LRRK2_Natguide | atatgctaatacgactcactatagGTGCCCTCTGATGTTTTTATC |
| | | CCCATTC#G#G#T#T#ACAGTA^{∧}ATGAGCAA*ATGC* |
| | | C*GAGTCAGCAA*G*ATTTGC#T#G#G#T#T#GGGCA |
| | | GCATTGGGATACAGTGTGAAAA |
| 59 | LRRK2_EIE | atatgctaatacgactcactatagAAGGACGGGTCGTACTGAGC |
| | | AATGC*C*CTAGTCA#A#A#G#T#G#G#A#C#AGCAA |
| | | TCTTTGCAA^{∧}CGATGGCAGCAT^{∧}CGGGATACA*C*C |
| | | TGTGACTAA |
| 60 | LRRK2_FlipIntGluR2 | atatgctaatacgactcactatagGTGCCCTCTGATGTTCTTATC |
| | | CCCATTCCACAGCAGTACTGAGCAATGC^{∧}CGTAGT |
| | | CACACCCTATGATATTGTTGTAAATCGTATAAC |
| | | AATATGATAAGGTGGCAATCTTCGCAATGATGG |
| | | CAGCATTGGGACACAGTGTGAAAA |
| 61 | LRRK2_IntGluR2 | atatgctaatacgacteactatagGTGCCCTCTGATGTTCTTATC |
| | | CCCATTCCACAGCAGTACTGAGCAATGC^{∧}CGTAGT |
| | | CAGTGGAATAGTATAACAATATGCTAAATGTTGTTATA |
| | | GTATCCCACGCAATCTTCGCAATGATGGCAGCATT |
| | | GGGATACAGTGTGAAAA |
| 62 | LRRK2_EIEv2 | atatgctaatacgactcactatagAAGGACGGGTCGTACTGAGC |
| | | AATG^{∧}CCGTAGTCA#A#A#G#T#G#G#A#C#AGCAAT |
| | | CTTTGCAA^{∧}CGATGGCAGCAT^{∧}CGGGATACA*C*CT |
| | | GTGACTAA |

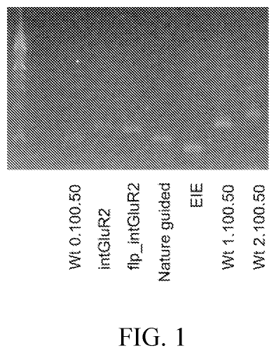

In vitro transcribed guide RNA (exemplary engineered polynucleotide sequences) was produced using gBlocks purchased from IDT. The IVT reaction is set up as listed in Table 11, as well as the primers in Table 12. IVT templates were made for all guides following Q5 PCR protocol (60° C. annealing) followed by confirmation via gel electrophoresis, FIG. 1.

TABLE 11
Exemplary IVT Protocol

| Reagent | Quantity | Concentration |
|---|---|---|
| Nuclease-free water | 5 μl | |
| 10X Reaction Buffer | 2 μl | |
| ATP (100 mM) | 2 μl | 10 mM final |
| GTP (100 mM) | 2 μl | 10 mM final |
| UTP (100 mM) | 2 μl | 10 mM final |
| CTP (100 mM) | 2 μl | 10 mM final |
| Template DNA | 3 μl | 1 μg |
| T7 RNA Polymerase Mix | 2 μl | |
| Total reaction volume | 20 μl | |

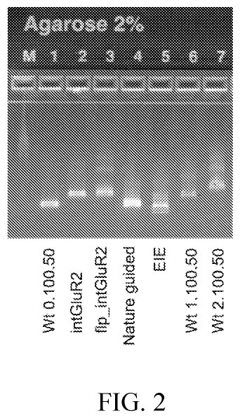

In brief, the IVT protocol shown in Table 11 was utilized to generate IVT guide RNA (exemplary engineered polynucleotide sequences). Reagents were mixed and incubated at 37° C. overnight (# overnight IVT generally gives a great yield). For the DNase treatment, the following were added: 70 μl nuclease-free water, 10 μl of 10× DNase I Buffer, and 2 μl of DNase I (RNase-free), were mixed and incubated for 30 minutes at 37° C. Column Purification is shown in FIG. 2. Purified IVT produced polynucleotide RNA was adjusted to 1 ug/ul (˜25 nmol).

TABLE 12
Exemplary IVT Primers

| SEQ | | |
|---|---|---|
| ID NO: | Primer | Sequence |
| 63 | LRRK2_RP_gen | TTTTCACACTGTATCCCAATG |
| 64 | LRRK2_RP_EIE | TTAGTCACAGGTGTATCCC |
| 65 | LRRK2_RP_intG2 | TTTTCACACTGTATCCCAATG |

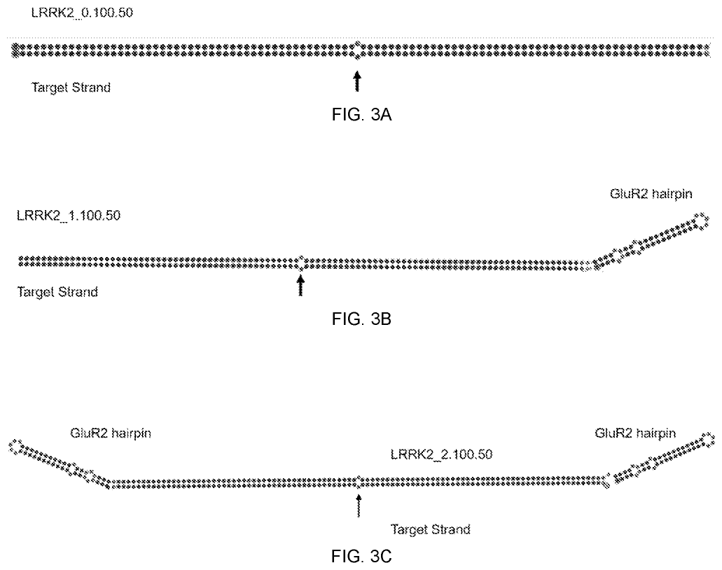

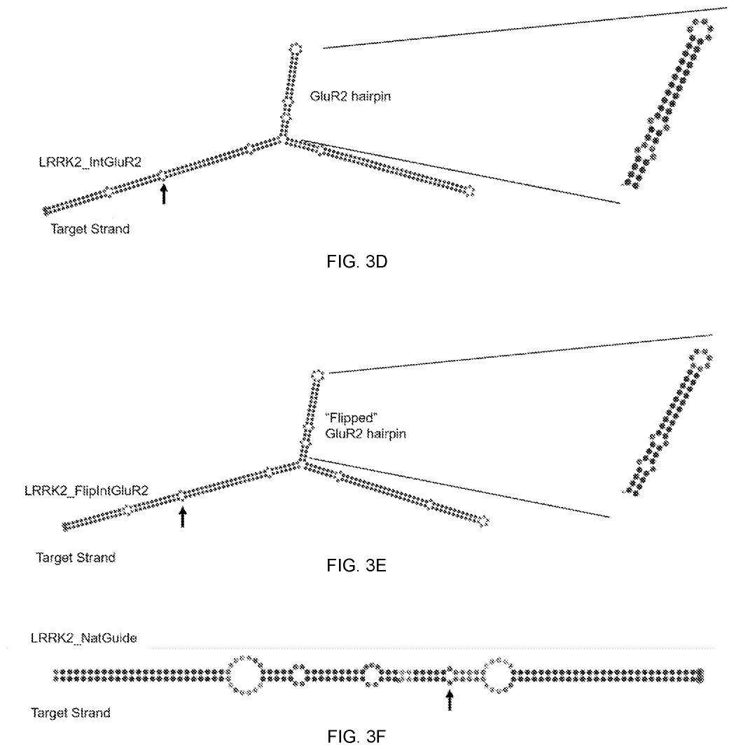

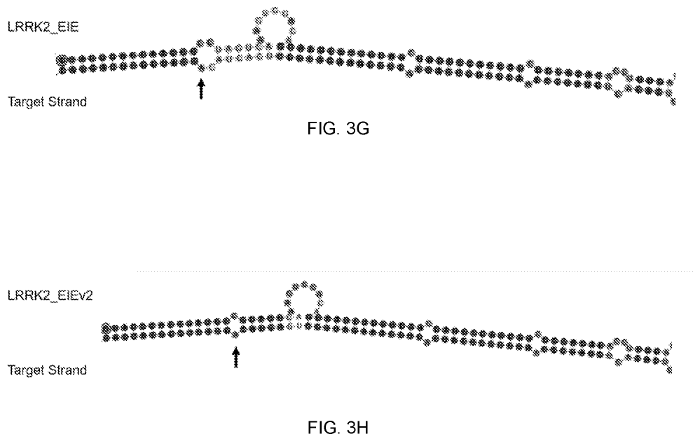

The secondary structures of the purified IVT produced polynucleotide RNA when hybridized with the target RNA strand are shown in FIGS. 3A-H.

Exemplary guide engineered polynucleotides are shown in Table 13. Suitable engineered polynucleotide sequences can comprise from about 60%, 70%, 80%, 85%, 90%, 95%, 96%, 97%, 98%, 99%, or 100% identity to any one of the sequences of Table 13. Exemplary engineered polynucleotides can facilitate the reversion of a single base pair mutation at position 6190 of the LRRK2 mRNA sequence of SEQ ID NO: 6.

TABLE 13
Exemplary engineered polynucleotide sequences and corresponding target
mRNA sequences (region of LRRK2 sequence targeted by the engineered polynucleotide).
Formatting indicates various elements of each construct:
Recruiting sequences (GluR2) are italicized; ^{∧} denotes a nucleotide mismatch; * denotes a
nucleotide that will form a bulge; # denotes a nucleotide that will form a loop; underlining
denotes a nucleotide that will form part of a hairpin.

| | | | mRNA sequence that is |
|---|---|---|---|
| | | | bound by the targeting |
| SEQ ID | | | sequence of the |
| NO: | ID | Engineered Polynucleotide Sequence | polynucleotide |
| 66 | LRRK2_0.100.50 | GUGCCCUCUGAUGUUUUUAUCCCCA | UUUUCACACUGUAUC |
| | | UUCUACAGCAGUACUGAGCAAUGC^{∧} | CCAAUGCUGCCAUCA |
| | | CGUAGUCAGCAAUCUUUGCAAUGAU | UUGCAAAGAUUGCUG |
| | | GGCAGCAUUGGGAUACAGUGUGAAA | ACUACAGCAUUGCUC |
| | | A | AGUACUGCUGUAGAA |
| | | | UGGGGAUAAAAACAU |
| | | | CAGAGGGCAC (SEQ |
| | | | ID NO: 73) |
| 67 | LRRK2_1.100.50 | GUGCCCUCUGAUGUUUUUAUCCCCA | SEQ ID NO: 73 |
| | | UUCUACAGCAGUACUGAGCAAUGC^{∧} | |
| | | CGUAGUCAGCAAUCUUUGCAAUGAU | |
| | | GGCAGCAUUGGGAUACAGUGUGAAA | |
| | | AGUGGAAUAGUAUAACAAUAUGCUAA | |
| | | AUGUUGUUAUAGUAUCCCAC | |
| 68 | LRRK2_2.100.50 | GUGGAAUAGUAUAACAAUAUGCUAAA | SEQ ID NO: 73 |
| | | UGUUGUUAUAGUAUCCCACGUGCCCU | |
| | | CUGAUGUUUUUAUCCCCAUUCUACA | |
| | | GCAGUACUGAGCAAUGC^{∧}CGUAGUC | |
| | | AGCAAUCUUUGCAAUGAUGGCAGCA | |
| | | UUGGGAUACAGUGUGAAAAGUGGAA | |
| | | UAGUAUAACAAUAUGCUAAAUGUUGU | |
| | | UAUAGUAUCCCAC | |
| 69 | LRRK2_Natguide | GUGCCCUCUGAUGUUUUUAUCCCCA | SEQ ID NO: 73 |
| | | UUC#G#G#U#U#ACAGUA^{∧}AUGAGCA | |
| | | A*AUGC*C*GAGUCAGCAA*G*AUUU | |
| | | GC#U#G#G#U#U#GGGCAGCAUUGGG | |
| | | AUACAGUGUGAAAA | |
| 70 | LRRK2_EIE | AAGGACGGGUCGUACUGAGCAAUGC | UGUAUCCCAAUGCUG |
| | | *C*CUAGUCA#A#A#G#U#G#G#A#C#A | CCAUCAUUGCAAAGA |
| | | GCAAUCUUUGCAA^{∧}CGAUGGCAGCA | UUGCUGACUACAGCA |
| | | U^{∧}CGGGAUACA*C*CUGUGACUAA | UUGCUCAGUAC (SEQ |
| | | | ID NO: 74) |
| 71 | LRRK2_ | GUGCCCUCUGAUGUUCUUAUCCCCA | SEQ ID NO: 74 |
| | FlipIntGluR2 | UUC^{∧}CACAGCAGUACUGAGCAAUGC^{A} | |
| | | CGUAGUCACACCCUAUGAUAUUGUU | |
| | | GUAAAUCGUAUAACAAUAUGAUAAGG | |
| | | UGGCAAUCUU^{∧}CGCAAUGAUGGCAG | |
| | | CAUUGGGA^{∧}CACAGUGUGAAAA | |
| 72 | LRRK2_IntGluR2 | GUGCCCUCUGAUGUUCUUAUCCCCA | SEQ ID NO: 74 |
| | | UUC*CACAGCAGUACUGAGCAAUGC^{A} | |
| | | CGUAGUCAGUGGAAUAGUAUAACAAU | |
| | | AUGCUAAAUGUUGUUAUAGUAUCCCA | |
| | | CGCAAUCUU^{∧}CGCAAUGAUGGCAGC | |
| | | AUUGGGAUA^{∧}CAGUGUGAAAA | |

### Example 2: Introduction of IVT Guide RNA into Cells Containing the G2019S LRRK2 Mutation

EBV transformed B cells, LCLs, containing one mutant allele encoding a G2019S mutation in LRRK2 (heterozygous), were purchased from the Coriell Institute. In particular, donor ND000264 was used throughout these experiments to assess ADAR mediated editing, reversion to wild-type allele, A>G conversion.

7 IVT generated engineered polynucleotides (e.g., guides) were tested against LRRK2 RNA as well as 1 IVT engineered polynucleotide (e.g., guide) against RAB7A RNA. All engineered polynucleotides were nucleofected in LCL cells using the Lonza X nucleofector, with program EH100. Either approximately 40 nmol or 60 nmol of each IVT generated engineered polynucleotide were nucleofected into about 2×10⁵ LCL cells per reaction condition. The reaction was split into 2 wells, containing 1×10⁵ each so that cells were collected for RNA isolation at either 3 hrs or 7 hrs. At collection, cells were spun at 1,500×g for 1 min, media was then removed, and then 180 ul of RLT buffer+BMe was added to each well. A Qiagen RNeasy protocol and kit were used to isolate RNA, followed by the New England Biolabs (NEB) ProtoScript II First-Strand cDNA synthesis kit.

LRRK2 mRNA specific primers, outside of the target regions, were used to amplify the region that the IVT generated engineered polynucleotides were targeting, Table 14. These primers have no sequence overlap with any of the IVT generated engineered polynucleotides. LRRK2 primer 1 (LRRK2_1) and 2 (LRRK2_2) were used to amplify the mRNA, and primer 4 (LRRK2_4) was used for sequencing of the target region. Sequencing was outsourced to Genewhiz. Sanger traces were analyzed to assess editing efficiency of each IVT generated engineered polynucleotide.

TABLE 14
LRRK2 mRNA specific primers

| SEQ | | |
|---|---|---|
| ID NO | Primer Name | Sequence |
| 75 | LRRK2_1 | CGTAGCTGATGGTTTGAGATACCT |
| 76 | LRRK2_2 | ACCAAATGAATAAACATCAGCCTGTTG |
| 77 | LRRK2_4 | TTTCCTCTGGCAACTTCAGGTG |

### Example 3: Correction of Pathogenic G2019S Mutation in the Cell by Engineered Polynucleotides Targeting LRRK2

Sequencing can comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, Enigma sequencing, or any combination thereof.

Different exemplary engineered polynucleotides (e.g., guide RNAs), such as LRRK2_FlipIntGluR2 (SEQ ID NO: 71, labeled as IntFlip), LRRK2_0.100.50 (SEQ ID NO: 66, labeled as 0.100.50), LRRK2_1.100.50 (SEQ ID NO: 67, labeled as 1.100.50), and control engineered polynucleotides were used to edit a LRRK2 mRNA having an A mutation at position 6055, which encodes for a pathogenic G2019S mutant protein. EBV transformed B cells heterozygous for the allele encoding a G2019S mutation were treated with the engineered polynucleotides.

All engineered polynucleotides were nucleofected in LCL cells using the Lonza X nucleofector, with program EH100. ˜40 nmol or 60 nmol of each IVT engineered polynucleotide was nucleofected into ˜2×10\hat{ }5 LCL cells per reaction condition. The reaction was split into 2 wells, containing 1×10\hat{ }5 each so that the cells could be collected for RNA isolation at either 3 hrs or 7 hrs. At collection, cells were spun at 1,500×g for 1 min. The media was then removed. 180 ul of RLT buffer+BMe was added to each well. Qiagen RNeasy protocol and kit were used to isolate the RNAs from the cell. New England Biolabs (NEB) ProtoScript II First-Strand cDNA synthesis kit was used to synthesize cDNA from the isolated RNA. cDNA of LRRK2 was sequenced by the Sanger sequencing. Sequencing was outsourced to Genewhiz. Sanger traces were analyzed to assess the editing efficiency of each engineered polynucleotide.

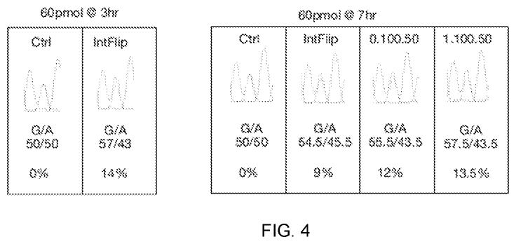

The RNA editing efficiency, the A>G conversion in LRRK2, was calculated by the difference of trace signal of the LRRK2 mRNA with a G (edited) and LRRK2 mRNA with an A (unedited) at the 6055^{th} nucleotide. As shown in FIG. 4, LRRK2_FlipIntGluR2 was able to achieve 14% editing efficiency, converting 7% of the pathogenic A mutation into G. By 7 hours, both LRRK2_0.100.50 and LRRK2_1.100.50 also showed comparable editing efficiency.

### Example 4: Gene Therapy for Correcting the Pathogenic G2019S Mutation in the Parkinson's Disease Patient by Engineered Polynucleotides Targeting LRRK2 mRNA

Parkinson patients diagnosed with the G2019S mutation can be treated with any of the engineered polynucleotides (e.g., guide RNAs) listed in Table 13. The engineered polynucleotides (e.g., guide RNAs) can be prepared by various methods. For example, the engineered polynucleotide (e.g., guide RNA) can be prepared by PCR and IVT, as illustrated in Example 1 and Example 2, and directly injected into the brain of the patient by intracerebroventricular injection.

The sequences of engineered polynucleotides (e.g., guide RNAs)—as listed in Table 10 with their T7 promoter sequence replaced with a U7, a U1, a U6, an H1, or a 7SK promoter sequence, can also be cloned into a viral vector, such as an adenoviral vector, an adeno-associated viral vector (AAV), a lentiviral vector, or a retroviral vector. The viral vector with the sequence of the engineered polynucleotide (e.g., guide RNA) can be directly injected into the brain of the patient by intracerebroventricular injection. The sequence of the engineered polynucleotide (e.g., guide RNA)—as listed in Table 10 with their T7 promoter sequence replaced with a U7, a U1, a U6, an H1, or a 7SK promoter sequence can be prepared by PCR or gBlocks™ Gene Fragments. The sequence of the engineered polynucleotide (e.g., guide RNA) can then be attached to nanoparticle or dendrimer for intracerebroventricular injection into the brain of the patient.

RNA editing is monitored as follows: —1×10\hat{ }5 are collected for RNA isolation after a week. At collection, cells are spun at 1,500×g for 1 min. The media are removed. 180 ul of RLT buffer+BMe is added to each well. Qiagen RNeasy protocol and kit are used to isolate the RNAs from the cell. New England Biolabs (NEB) ProtoScript II First-Strand cDNA synthesis kit is used to synthesize cDNA from the isolated RNA. cDNA of LRRK2 is sequenced by the Sanger sequencing. Sanger traces are analyzed to assess the editing efficiency of each engineered polynucleotide (e.g., IVT guide). The RNA editing efficiency, the A>G conversion in LRRK2 mRNA, is calculated by the difference of trace signal of the LRRK2 mRNA with a G (edited) and an A (unedited) at the 6055^{th} nucleotide.

### Example 5: Gene Therapy for Correcting the Pathogenic Mutation(s) in the Crohn's Disease Patient by Engineered Polynucleotides Targeting LRRK2 mRNA

Crohn's disease patient with pathogenic mutations can be treated by gene therapy with engineered polynucleotide (e.g., guide RNA) targeting LRRK2 mRNA. If a patient is diagnosed with the G2019S mutation, engineered polynucleotides (e.g., guide RNAs) illustrated in Example 1-4 can be used for gene therapy. If a Crohn's disease patient is diagnosed with another mutation, such as the N2081D mutation, other engineered polynucleotide (e.g., guide RNA) will be synthesized. The engineered polynucleotide (e.g., guide RNA) will be constructed to convert A into G at the 6243^{th} nucleotide for RNA encoding a N2081D mutant protein. The engineered polynucleotide (e.g., guide RNA) can be prepared by PCR and IVT and intravenously injected into a patient.

The sequence of the engineered polynucleotide (e.g., guide RNA) with an upstream U7, a U1, a U6, an H1, or a 7SK promoter can also be cloned into a viral vector, such as an adenoviral vector, an adeno-associated viral vector (AAV), a lentiviral vector, or a retroviral vector, or any exemplary vector as provided in Table 15. The viral vector comprising a sequence encoding the engineered polynucleotide (e.g., guide RNA) can be intravenously injected into a patient.

The sequence of the engineered polynucleotide (e.g., guide RNA) with an upstream U7, a U1, a U6, an H1, or a 7SK promoter can also be prepared by PCR or gBlocks™ Gene Fragments. The sequence of the engineered polynucleotide (e.g., guide RNA) can then be attached to nanoparticles or dendrimers for intraveneous injection into the patient.

RNA editing is monitored as follows: —1×10\hat{ }5 are collected for RNA isolation after a week. At collection, cells are spun at 1,500×g for 1 min. The media are removed. 180 ul of RLT buffer+BMe is added to each well. Qiagen RNeasy protocol and kit are used to isolate the RNAs from the cell. New England Biolabs (NEB) ProtoScript II First-Strand cDNA synthesis kit is used to synthesize cDNA from the isolated RNA. cDNA of LRRK2 is sequenced by the Sanger sequencing. Sanger traces are analyzed to assess the editing efficiency of each IVT guide. The RNA editing efficiency, the A>G conversion in LRRK2, is calculated by the difference of trace signal of the LRRK2 mRNA with a G (edited) and an A (unedited) at the 6243^{th} nucleotide.

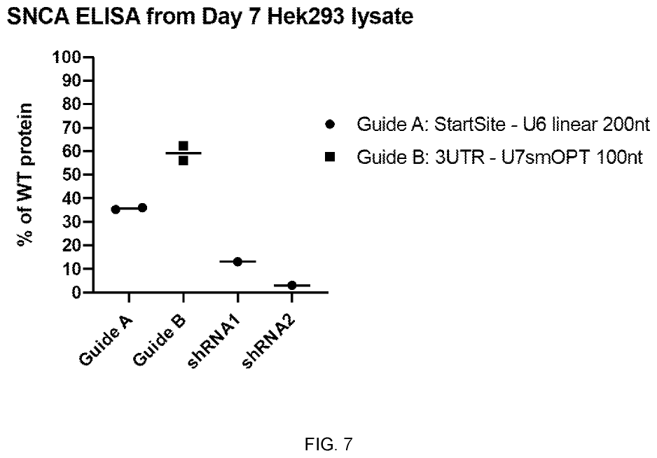

TABLE 15
Exemplary Vectors that can encode subject polynucleotides

| SEQ ID | | |
|---|---|---|
| NO: | ID | Sequence |
| 78 | STB026 mU7 GG | TCGCGCGTTTCGGTGATGACGGTGAAAACCTCTGACA |
| | U7SmOpt_mU6_CMV | CATGCAGCTCCCGGAGACGGTCACAGCTTGTCTGTAA |
| | GFP sv40 (FIG. 7A) | GCGGATGCCGGGAGCAGACAAGCCCGTCAGGGCGCG |
| | | TCAGCGGGTGTTGGCGGGTGTCGGGGCTGGCTTAACT |
| | | ATGCGGCATCAGAGCAGATTGTACTGAGAGTGCACC |
| | | ATATGCGGTGTGAAATACCGCACAGATGCGTAAGGA |
| | | GAAAATACCGCATCAGGCGCCATTCGCCATTCAGGC |
| | | TGCGCAACTGTTGGGAAGGGCGATCGGTGCGGGCCT |
| | | CTTCGCTATTACGCCAGCTGGCGAAAGGGGGATGTG |
| | | CTGCAAGGCGATTAAGTTGGGTAACGCCAGGGTTTTC |
| | | CCAGTCACGACGTTGTAAAACGACGGCCAGTGAATT |
| | | GACGCGCCATTGGGATGTTGTAAAACGACGGCCAGT |
| | | GAACCTGCAGGCAGCTGCGCGCTCGCTCGCTCACTG |
| | | AGGCCGCCCGGGCAAAGCCCGGGCGTCGGGCGACCT |
| | | TTGGTCGCCCGGCCTCAGTGAGCGAGCGAGCGCGCA |
| | | GAGAGGGAGTGGCCAACTCCATCACTAGGGGTTCCT |
| | | GCGGCCGCACGCGTTAACAACATAGGAGCTGTGATT |
| | | GGCTGTTTTCAGCCAATCAGCACTGACTCATTTGCAT |
| | | AGCCTTTACAAGCGGTCACAAACTCAAGAAACGAGC |
| | | GGTTTTAATAGTCTTTTAGAATATTGTTTATCGAACC |
| | | GAATAAGGAACTGTGCTTTGTGATTCACATATCAGTG |
| | | GAGGGGTGTGGAAATGGCACCTTGATCTCACCCTCAT |
| | | CGAAAGTGGAGTTGATGTCCTTCCCTGGCTCGCTACA |
| | | GACGCACTTCCGCACCGAGAGACCAGCTGGATGGTC |
| | | TCAGTGGAATTTTTGGAGCAGGTTTTCTGACTTCGGT |
| | | CGGAAAACCCCTCCCAATTTCACTGGTCTACAATGAA |
| | | AGCAAAACAGTTCTCTTCCCCGCTCCCCGGTGTGTGA |
| | | GAGGGGCTTTGATCCTTCTCTGGTTTCCTAGGAAACG |
| | | CGTATGTGCTAGCGTACTGAGTCGCCCAGTCTCAGAT |
| | | AGATCCGACGCCGCCATCTCTAGGCCCGCGCCGGCC |
| | | CCCTCGCACAGACTTGTGGGAGAAGCTCGGCTACTCC |
| | | CCTGCCCCGGTTAATTTGCATATAATATTTCCTAGTA |
| | | ACTATAGAGGCTTAATGTGCGATAAAAGACAGATAA |
| | | TCTGTTCTTTTTAATACTAGCTACATTTTACATGATAG |
| | | GCTTGGATTTCTATAAGAGATACAAATACTAAATTAT |
| | | TATTTTAAAAAACAGCACAAAAGGAAACTCACCCTA |
| | | ACTGTAAAGTAATTGTGTGTTTTGAGACTATAAATAT |
| | | CCCTTGGAGAAAAGCCTTGTTTGGAATTCATACGCGT |
| | | TGACATTGATTATTGACTAGTTATTAATAGTAATCAA |
| | | TTACGGGGTCATTAGTTCATAGCCCATATATGGAGTT |
| | | CCGCGTTACATAACTTACGGTAAATGGCCCGCCTGGC |
| | | TGACCGCCCAACGACCCCCGCCCATTGACGTCAATA |
| | | ATGACGTATGTTCCCATAGTAACGCCAATAGGGACTT |
| | | TCCATTGACGTCAATGGGTGGAGTATTTACGGTAAAC |
| | | TGCCCACTTGGCAGTACATCAAGTGTATCATATGCCA |
| | | AGTACGCCCCCTATTGACGTCAATGACGGTAAATGG |
| | | CCCGCCTGGCATTATGCCCAGTACATGACCTTATGGG |
| | | ACTTTCCTACTTGGCAGTACATCTACGTATTAGTCAT |
| | | CGCTATTACCATGGTGATGCGGTTTTGGCAGTACATC |
| | | AATGGGCGTGGATAGCGGTTTGACTCACGGGGATTT |
| | | CCAAGTCTCCACCCCATTGACGTCAATGGGAGTTTGT |
| | | TTTGGCACCAAAATCAACGGGACTTTCCAAAATGTCG |
| | | TAACAACTCCGCCCCATTGACGCAAATGGGCGGTAG |
| | | GCGTGTACGGTGGGAGGTCTATATAAGCAGAGCTCG |
| | | TTTAGTGAACCGTCAGATCGCCTGGAGACGCCATCCA |
| | | CGCTGTTTTGACCTCCATAGAAGACACCGGGACCGAT |
| | | CCAGCCTCCGGACTCTAGAGGATCGAACCCTTAAGC |
| | | CGCCACCATGGTGAGCAAGGGCGAGGAGCTGTTCAC |
| | | CGGGGTGGTGCCCATCCTGGTCGAGCTGGACGGCGA |
| | | CGTAAACGGCCACAAGTTCAGCGTGTCCGGCGAGGG |
| | | CGAGGGCGATGCCACCTACGGCAAGCTGACCCTGAA |
| | | GTTCATCTGCACCACCGGCAAGCTGCCCGTGCCCTGG |
| | | CCCACCCTCGTGACCACCCTGACCTACGGCGTGCAGT |
| | | GCTTCAGCCGCTACCCCGACCACATGAAGCAGCACG |
| | | ACTTCTTCAAGTCCGCCATGCCCGAAGGCTACGTCCA |
| | | GGAGCGCACCATCTTCTTCAAGGACGACGGCAACTA |
| | | CAAGACCCGCGCCGAGGTGAAGTTCGAGGGCGACAC |
| | | CCTGGTGAACCGCATCGAGCTGAAGGGCATCGACTT |
| | | CAAGGAGGACGGCAACATCCTGGGGCACAAGCTGGA |
| | | GTACAACTACAACAGCCACAACGTCTATATCATGGC |
| | | CGACAAGCAGAAGAACGGCATCAAGGTGAACTTCAA |
| | | GATCCGCCACAACATCGAGGACGGCAGCGTGCAGCT |
| | | CGCCGACCACTACCAGCAGAACACCCCCATCGGCGA |
| | | CGGCCCCGTGCTGCTGCCCGACAACCACTACCTGAG |
| | | CACCCAGTCCGCCCTGAGCAAAGACCCCAACGAGAA |
| | | GCGCGATCACATGGTCCTGCTGGAGTTCGTGACCGCC |
| | | GCCGGGATCACTCTCGGCATGGACGAGCTGTACAAG |
| | | TACTCAGATCTCGAGCTCAAGTGAACCGGTCAGACA |
| | | TGATAAGATACATTGATGAGTTTGGACAAACCACAA |
| | | CTAGAATGCAGTGAAAAAAATGCTTTATTTGTGAAAT |
| | | TTGTGATGCTATTGCTTTATTTGTAACCATTATAAGCT |
| | | GCAATAAACAAGTTAACAACAACAATTGCATTCATTT |
| | | TATGTTTCAGGTTCAGGGGGAGGTGTGGGAGGTTTTT |
| | | TAAACACGTGCGGACCGAGCGGCCGCAGGAACCCCT |
| | | AGTGATGGAGTTGGCCACTCCCTCTCTGCGCGCTCGC |
| | | TCGCTCACTGAGGCCGGGCGACCAAAGGTCGCCCGA |
| | | CGCCCGGGCTTTGCCCGGGCGGCCTCAGTGAGCGAG |
| | | CGAGCGCGCAGCTGCCTGCAGGCTTGGATCCCAATG |
| | | GCGCGCCGAGCTTGGCTCGAGCATGGTCATAGCTGTT |
| | | TCCTGTGTGAAATTGTTATCCGCTCACAATTCCACAC |
| | | AACATACGAGCCGGAAGCATAAAGTGTAAAGCCTGG |
| | | GGTGCCTAATGAGTGAGCTAACTCACATTAATTGCGT |
| | | TGCGCTCACTGCCCGCTTTCCAGTCGGGAAACCTGTC |
| | | GTGCCAGCTGCATTAATGAATCGGCCAACGCGCGGG |
| | | GAGAGGCGGTTTGCGTATTGGGCGCTCTTCCGCTTCC |
| | | TCGCTCACTGACTCGCTGCGCTCGGTCGTTCGGCTGC |
| | | GGCGAGCGGTATCAGCTCACTCAAAGGCGGTAATAC |
| | | GGTTATCCACAGAATCAGGGGATAACGCAGGAAAGA |
| | | ACATGTGAGCAAAAGGCCAGCAAAAGGCCAGGAAC |
| | | CGTAAAAAGGCCGCGTTGCTGGCGTTTTTCCATAGGC |
| | | TCCGCCCCCCTGACGAGCATCACAAAAATCGACGCT |
| | | CAAGTCAGAGGTGGCGAAACCCGACAGGACTATAAA |
| | | GATACCAGGCGTTTCCCCCTGGAAGCTCCCTCGTGCG |
| | | CTCTCCTGTTCCGACCCTGCCGCTTACCGGATACCTG |
| | | TCCGCCTTTCTCCCTTCGGGAAGCGTGGCGCTTTCTC |
| | | ATAGCTCACGCTGTAGGTATCTCAGTTCGGTGTAGGT |
| | | CGTTCGCTCCAAGCTGGGCTGTGTGCACGAACCCCCC |
| | | GTTCAGCCCGACCGCTGCGCCTTATCCGGTAACTATC |
| | | GTCTTGAGTCCAACCCGGTAAGACACGACTTATCGCC |
| | | ACTGGCAGCAGCCACTGGTAACAGGATTAGCAGAGC |
| | | GAGGTATGTAGGCGGTGCTACAGAGTTCTTGAAGTG |
| | | GTGGCCTAACTACGGCTACACTAGAAGAACAGTATT |
| | | TGGTATCTGCGCTCTGCTGAAGCCAGTTACCTTCGGA |
| | | AAAAGAGTTGGTAGCTCTTGATCCGGCAAACAAACC |
| | | ACCGCTGGTAGCGGTGGTTTTTTTGTTTGCAAGCAGC |
| | | AGATTACGCGCAGAAAAAAAGGATCTCAAGAAGATC |
| | | CTTTGATCTTTTCTACGGGGTCTGACGCTCAGTGGAA |
| | | CGAAAACTCACGTTAAGGGATTTTGGTCATGAGATTA |
| | | TCAAAAAGGATCTTCACCTAGATCCTTTTAAATTAAA |
| | | AATGAAGTTTTAAATCAATCTAAAGTATATATGAGTA |
| | | AACTTGGTCTGACAGTTAGAAAAACTCATCGAGCAT |
| | | CAAATGAAACTGCAATTTATTCATATCAGGATTATCA |
| | | ATACCATATTTTTGAAAAAGCCGTTTCTGTAATGAAG |
| | | GAGAAAACTCACCGAGGCAGTTCCATAGGATGGCAA |
| | | GATCCTGGTATCGGTCTGCGATTCCGACTCGTCCAAC |
| | | ATCAATACAACCTATTAATTTCCCCTCGTCAAAAATA |
| | | AGGTTATCAAGTGAGAAATCACCATGAGTGACGACT |
| | | GAATCCGGTGAGAATGGCAAAAGTTTATGCATTTCTT |
| | | TCCAGACTTGTTCAACAGGCCAGCCATTACGCTCGTC |
| | | ATCAAAATCACTCGCATCAACCAAACCGTTATTCATT |
| | | CGTGATTGCGCCTGAGCGAGACGAAATACGCGATCG |
| | | CTGTTAAAAGGACAATTACAAACAGGAATCGAATGC |
| | | AACCGGCGCAGGAACACTGCCAGCGCATCAACAATA |
| | | TTTTCACCTGAATCAGGATATTCTTCTAATACCTGGA |
| | | ATGCTGTTTTCCCAGGGATCGCAGTGGTGAGTAACCA |
| | | TGCATCATCAGGAGTACGGATAAAATGCTTGATGGT |
| | | CGGAAGAGGCATAAATTCCGTCAGCCAGTTTAGTCT |
| | | GACCATCTCATCTGTAACATCATTGGCAACGCTACCT |
| | | TTGCCATGTTTCAGAAACAACTCTGGCGCATCGGGCT |
| | | TCCCATACAATCGATAGATTGTCGCACCTGATTGCCC |
| | | GACATTATCGCGAGCCCATTTATACCCATATAAATCA |
| | | GCATCCATGTTGGAATTTAATCGCGGCCTAGAGCAA |
| | | GACGTTTCCCGTTGAATATGGCTCATACTCTTCCTTTT |
| | | TCAATATTATTGAAGCATTTATCAGGGTTATTGTCTC |
| | | ATGAGCGGATACATATTTGAATGTATTTAGAAAAAT |
| | | AAACAAATAGGGGTTCCGCGCACATTTCCCCGAAAA |
| | | GTGCCACCTGACGTCTAAGAAACCATTATTATCATGA |
| | | CATTAACCTATAAAAATAGGCGTATCACGAGGCCCTT |
| | | TCGTC |
| 79 | STX0364 | TCGCGCGTTTCGGTGATGACGGTGAAAACCTCTGACA |
| | pAAV_hU6_scarless- | CATGCAGCTCCCGGAGACGGTCACAGCTTGTCTGTAA |
| | BsaI_mU6- | GCGGATGCCGGGAGCAGACAAGCCCGTCAGGGCGCG |
| | BbsI_CMV_GFP_no_ | TCAGCGGGTGTTGGCGGGTGTCGGGGCTGGCTTAACT |
| | BbsI_ (FIG. 7B) | ATGCGGCATCAGAGCAGATTGTACTGAGAGTGCACC |
| | | ATATGCGGTGTGAAATACCGCACAGATGCGTAAGGA |
| | | GAAAATACCGCATCAGGCGCCATTCGCCATTCAGGC |
| | | TGCGCAACTGTTGGGAAGGGCGATCGGTGCGGGCCT |
| | | CTTCGCTATTACGCCAGCTGGCGAAAGGGGGATGTG |
| | | CTGCAAGGCGATTAAGTTGGGTAACGCCAGGGTTTTC |
| | | CCAGTCACGACGTTGTAAAACGACGGCCAGTGAATT |
| | | GACGCGCCATTGGGATGTTGTAAAACGACGGCCAGT |
| | | GAACCTGCAGGCAGCTGCGCGCTCGCTCGCTCACTG |
| | | AGGCCGCCCGGGCAAAGCCCGGGCGTCGGGCGACCT |
| | | TTGGTCGCCCGGCCTCAGTGAGCGAGCGAGCGCGCA |
| | | GAGAGGGAGTGGCCAACTCCATCACTAGGGGTTCCT |
| | | GCGGCCGCACGCGTGGAGGAGGGCCTATTTCCCATG |
| | | ATTCCTTCATATTTGCATATACGATACAAGGCTGTTA |
| | | GAGAGATAATTAGAATTAATTTGACTGTAAACACAA |
| | | AGATATTAGTACAAAATACGTGACGTAGAAAGTAAT |
| | | AATTTCTTGGGTAGTTTGCAGTTTTAAAATTATGTTTT |
| | | AAAATGGACTATCATATGCTTACCGTAACTTGAAAGT |
| | | ATTTCGATTTCTTGGCTTTATATATCTTGTGGAAAGG |
| | | ACGAAACACCGAGAGACCAGCTGGATGGTCTCTTTTT |
| | | TTTCGTACTGAGTCGCCCAGTCTCAGATAGATCCGAC |
| | | GCCGCCATCTCTAGGCCCGCGCCGGCCCCCTCGCACA |
| | | GACTTGTGGGAGAAGCTCGGCTACTCCCCTGCCCCGG |
| | | TTAATTTGCATATAATATTTCCTAGTAACTATAGAGG |
| | | CTTAATGTGCGATAAAAGACAGATAATCTGTTCTTTT |
| | | TAATACTAGCTACATTTTACATGATAGGCTTGGATTT |
| | | CTATAAGAGATACAAATACTAAATTATTATTTTAAAA |
| | | AACAGCACAAAAGGAAACTCACCCTAACTGTAAAGT |
| | | AATTGTGTGTTTTGAGACTATAAATATCCCTTGGAGA |
| | | AAAGCCTTGTTTGGATGTCTTCACAGGAAGACGCTTT |
| | | TTTTGCGGCCGCATACGCGTTGACATTGATTATTGAC |
| | | TAGTTATTAATAGTAATCAATTACGGGGTCATTAGTT |
| | | CATAGCCCATATATGGAGTTCCGCGTTACATAACTTA |
| | | CGGTAAATGGCCCGCCTGGCTGACCGCCCAACGACC |
| | | CCCGCCCATTGACGTCAATAATGACGTATGTTCCCAT |
| | | AGTAACGCCAATAGGGACTTTCCATTGACGTCAATG |
| | | GGTGGAGTATTTACGGTAAACTGCCCACTTGGCAGTA |
| | | CATCAAGTGTATCATATGCCAAGTACGCCCCCTATTG |
| | | ACGTCAATGACGGTAAATGGCCCGCCTGGCATTATG |
| | | CCCAGTACATGACCTTATGGGACTTTCCTACTTGGCA |
| | | GTACATCTACGTATTAGTCATCGCTATTACCATGGTG |
| | | ATGCGGTTTTGGCAGTACATCAATGGGCGTGGATAG |
| | | CGGTTTGACTCACGGGGATTTCCAAGTCTCCACCCCA |
| | | TTGACGTCAATGGGAGTTTGTTTTGGCACCAAAATCA |
| | | ACGGGACTTTCCAAAATGTCGTAACAACTCCGCCCCA |
| | | TTGACGCAAATGGGCGGTAGGCGTGTACGGTGGGAG |
| | | GTCTATATAAGCAGAGCTCGTTTAGTGAACCGTCAGA |
| | | TCGCCTGGAGACGCCATCCACGCTGTTTTGACCTCCA |
| | | TAGTAGACACCGGGACCGATCCAGCCTCCGGACTCT |
| | | AGAGGATCGAACCCTTAAGCCGCCACCATGGTGAGC |
| | | AAGGGCGAGGAGCTGTTCACCGGGGTGGTGCCCATC |
| | | CTGGTCGAGCTGGACGGCGACGTAAACGGCCACAAG |
| | | TTCAGCGTGTCCGGCGAGGGCGAGGGCGATGCCACC |
| | | TACGGCAAGCTGACCCTGAAGTTCATCTGCACCACCG |
| | | GCAAGCTGCCCGTGCCCTGGCCCACCCTCGTGACCAC |
| | | CCTGACCTACGGCGTGCAGTGCTTCAGCCGCTACCCC |
| | | GACCACATGAAGCAGCACGACTTCTTCAAGTCCGCC |
| | | ATGCCCGAAGGCTACGTCCAGGAGCGCACCATCTTCT |
| | | TCAAGGACGACGGCAACTACAAGACCCGCGCCGAGG |
| | | TGAAGTTCGAGGGCGACACCCTGGTGAACCGCATCG |
| | | AGCTGAAGGGCATCGACTTCAAGGAGGACGGCAACA |
| | | TCCTGGGGCACAAGCTGGAGTACAACTACAACAGCC |
| | | ACAACGTCTATATCATGGCCGACAAGCAGAAGAACG |
| | | GCATCAAGGTGAACTTCAAGATCCGCCACAACATCG |
| | | AGGACGGCAGCGTGCAGCTCGCCGACCACTACCAGC |
| | | AGAACACCCCCATCGGCGACGGCCCCGTGCTGCTGC |
| | | CCGACAACCACTACCTGAGCACCCAGTCCGCCCTGA |
| | | GCAAAGACCCCAACGAGAAGCGCGATCACATGGTCC |
| | | TGCTGGAGTTCGTGACCGCCGCCGGGATCACTCTCGG |
| | | CATGGACGAGCTGTACAAGTACTCAGATCTCGAGCT |
| | | CAAGTGAACCGGTCAGACATGATAAGATACATTGAT |
| | | GAGTTTGGACAAACCACAACTAGAATGCAGTGAAAA |
| | | AAATGCTTTATTTGTGAAATTTGTGATGCTATTGCTTT |
| | | ATTTGTAACCATTATAAGCTGCAATAAACAAGTTAAC |
| | | AACAACAATTGCATTCATTTTATGTTTCAGGTTCAGG |
| | | GGGAGGTGTGGGAGGTTTTTTAAACACGTGCGGACC |
| | | GAGCGGCCGCAGGAACCCCTAGTGATGGAGTTGGCC |
| | | ACTCCCTCTCTGCGCGCTCGCTCGCTCACTGAGGCCG |
| | | GGCGACCAAAGGTCGCCCGACGCCCGGGCTTTGCCC |
| | | GGGCGGCCTCAGTGAGCGAGCGAGCGCGCAGCTGCC |
| | | TGCAGGCTTGGATCCCAATGGCGCGCCGAGCTTGGCT |
| | | CGAGCATGGTCATAGCTGTTTCCTGTGTGAAATTGTT |
| | | ATCCGCTCACAATTCCACACAACATACGAGCCGGAA |
| | | GCATAAAGTGTAAAGCCTGGGGTGCCTAATGAGTGA |
| | | GCTAACTCACATTAATTGCGTTGCGCTCACTGCCCGC |
| | | TTTCCAGTCGGGAAACCTGTCGTGCCAGCTGCATTAA |
| | | TGAATCGGCCAACGCGCGGGGAGAGGCGGTTTGCGT |
| | | ATTGGGCGCTCTTCCGCTTCCTCGCTCACTGACTCGC |
| | | TGCGCTCGGTCGTTCGGCTGCGGCGAGCGGTATCAGC |
| | | TCACTCAAAGGCGGTAATACGGTTATCCACAGAATC |
| | | AGGGGATAACGCAGGAAAGAACATGTGAGCAAAAG |
| | | GCCAGCAAAAGGCCAGGAACCGTAAAAAGGCCGCGT |
| | | TGCTGGCGTTTTTCCATAGGCTCCGCCCCCCTGACGA |
| | | GCATCACAAAAATCGACGCTCAAGTCAGAGGTGGCG |
| | | AAACCCGACAGGACTATAAAGATACCAGGCGTTTCC |
| | | CCCTGGAAGCTCCCTCGTGCGCTCTCCTGTTCCGACC |
| | | CTGCCGCTTACCGGATACCTGTCCGCCTTTCTCCCTTC |
| | | GGGAAGCGTGGCGCTTTCTCATAGCTCACGCTGTAGG |
| | | TATCTCAGTTCGGTGTAGGTCGTTCGCTCCAAGCTGG |
| | | GCTGTGTGCACGAACCCCCCGTTCAGCCCGACCGCTG |
| | | CGCCTTATCCGGTAACTATCGTCTTGAGTCCAACCCG |
| | | GTAAGACACGACTTATCGCCACTGGCAGCAGCCACT |
| | | GGTAACAGGATTAGCAGAGCGAGGTATGTAGGCGGT |
| | | GCTACAGAGTTCTTGAAGTGGTGGCCTAACTACGGCT |
| | | ACACTAGAAGAACAGTATTTGGTATCTGCGCTCTGCT |
| | | GAAGCCAGTTACCTTCGGAAAAAGAGTTGGTAGCTC |
| | | TTGATCCGGCAAACAAACCACCGCTGGTAGCGGTGG |
| | | TTTTTTTGTTTGCAAGCAGCAGATTACGCGCAGAAAA |
| | | AAAGGATCTCAAGAAGATCCTTTGATCTTTTCTACGG |
| | | GGTCTGACGCTCAGTGGAACGAAAACTCACGTTAAG |
| | | GGATTTTGGTCATGAGATTATCAAAAAGGATCTTCAC |
| | | CTAGATCCTTTTAAATTAAAAATGAAGTTTTAAATCA |
| | | ATCTAAAGTATATATGAGTAAACTTGGTCTGACAGTT |
| | | AGAAAAACTCATCGAGCATCAAATGAAACTGCAATT |
| | | TATTCATATCAGGATTATCAATACCATATTTTTGAAA |
| | | AAGCCGTTTCTGTAATGAAGGAGAAAACTCACCGAG |
| | | GCAGTTCCATAGGATGGCAAGATCCTGGTATCGGTCT |
| | | GCGATTCCGACTCGTCCAACATCAATACAACCTATTA |
| | | ATTTCCCCTCGTCAAAAATAAGGTTATCAAGTGAGAA |
| | | ATCACCATGAGTGACGACTGAATCCGGTGAGAATGG |
| | | CAAAAGTTTATGCATTTCTTTCCAGACTTGTTCAACA |
| | | GGCCAGCCATTACGCTCGTCATCAAAATCACTCGCAT |
| | | CAACCAAACCGTTATTCATTCGTGATTGCGCCTGAGC |
| | | GAGACGAAATACGCGATCGCTGTTAAAAGGACAATT |
| | | ACAAACAGGAATCGAATGCAACCGGCGCAGGAACAC |
| | | TGCCAGCGCATCAACAATATTTTCACCTGAATCAGGA |
| | | TATTCTTCTAATACCTGGAATGCTGTTTTCCCAGGGA |
| | | TCGCAGTGGTGAGTAACCATGCATCATCAGGAGTAC |
| | | GGATAAAATGCTTGATGGTCGGAAGAGGCATAAATT |
| | | CCGTCAGCCAGTTTAGTCTGACCATCTCATCTGTAAC |
| | | ATCATTGGCAACGCTACCTTTGCCATGTTTCAGAAAC |
| | | AACTCTGGCGCATCGGGCTTCCCATACAATCGATAGA |
| | | TTGTCGCACCTGATTGCCCGACATTATCGCGAGCCCA |
| | | TTTATACCCATATAAATCAGCATCCATGTTGGAATTT |
| | | AATCGCGGCCTAGAGCAAGACGTTTCCCGTTGAATAT |
| | | GGCTCATACTCTTCCTTTTTCAATATTATTGAAGCATT |
| | | TATCAGGGTTATTGTCTCATGAGCGGATACATATTTG |
| | | AATGTATTTAGAAAAATAAACAAATAGGGGTTCCGC |
| | | GCACATTTCCCCGAAAAGTGCCACCTGACGTCTAAG |
| | | AAACCATTATTATCATGACATTAACCTATAAAAATAG |
| | | GCGTATCACGAGGCCCTTTCGTC |
| 80 | STX0441_pAAV_hU6_ | TCGCGCGTTTCGGTGATGACGGTGAAAACCTCTGACA |
| | circular- | CATGCAGCTCCCGGAGACGGTCACAGCTTGTCTGTAA |
| | spacer2A_mU6_CMV_ | GCGGATGCCGGGAGCAGACAAGCCCGTCAGGGCGCG |
| | GFP (FIG. 7C) | TCAGCGGGTGTTGGCGGGTGTCGGGGCTGGCTTAACT |
| | | ATGCGGCATCAGAGCAGATTGTACTGAGAGTGCACC |
| | | ATATGCGGTGTGAAATACCGCACAGATGCGTAAGGA |
| | | GAAAATACCGCATCAGGCGCCATTCGCCATTCAGGC |
| | | TGCGCAACTGTTGGGAAGGGCGATCGGTGCGGGCCT |
| | | CTTCGCTATTACGCCAGCTGGCGAAAGGGGGATGTG |
| | | CTGCAAGGCGATTAAGTTGGGTAACGCCAGGGTTTTC |
| | | CCAGTCACGACGTTGTAAAACGACGGCCAGTGAATT |
| | | GACGCGCCATTGGGATGTTGTAAAACGACGGCCAGT |
| | | GAACCTGCAGGCAGCTGCGCGCTCGCTCGCTCACTG |
| | | AGGCCGCCCGGGCAAAGCCCGGGCGTCGGGCGACCT |
| | | TTGGTCGCCCGGCCTCAGTGAGCGAGCGAGCGCGCA |
| | | GAGAGGGAGTGGCCAACTCCATCACTAGGGGTTCCT |
| | | GCGGCCGCACGCGTGGAGGAGGGCCTATTTCCCATG |
| | | ATTCCTTCATATTTGCATATACGATACAAGGCTGTTA |
| | | GAGAGATAATTAGAATTAATTTGACTGTAAACACAA |
| | | AGATATTAGTACAAAATACGTGACGTAGAAAGTAAT |
| | | AATTTCTTGGGTAGTTTGCAGTTTTAAAATTATGTTTT |
| | | AAAATGGACTATCATATGCTTACCGTAACTTGAAAGT |
| | | ATTTCGATTTCTTGGCTTTATATATCTTGTGGAAAGG |
| | | ACGAAACACCGGCCATCAGTCGCCGGTCCCAAGCCC |
| | | GGATAAAATGGGAGGGGGCGGGAAACCGCCTAACC |
| | | ATGCCGACTGATGGCAGATAATAGAGACCAGCTGGA |
| | | TGGTCTCAATATACTGCCATCAGTCGGCGTGGACTGT |
| | | AGAACACTGCCAATGCCGGTCCCAAGCCCGGATAAA |
| | | AGTGGAGGGTACAGTCCACGCTTTTTTTCGTACTGAG |
| | | TCGCCCAGTCTCAGATAGATCCGACGCCGCCATCTCT |
| | | AGGCCCGCGCCGGCCCCCTCGCACAGACTTGTGGGA |
| | | GAAGCTCGGCTACTCCCCTGCCCCGGTTAATTTGCAT |
| | | ATAATATTTCCTAGTAACTATAGAGGCTTAATGTGCG |
| | | ATAAAAGACAGATAATCTGTTCTTTTTAATACTAGCT |
| | | ACATTTTACATGATAGGCTTGGATTTCTATAAGAGAT |
| | | ACAAATACTAAATTATTATTTTAAAAAACAGCACAA |
| | | AAGGAAACTCACCCTAACTGTAAAGTAATTGTGTGTT |
| | | TTGAGACTATAAATATCCCTTGGAGAAAAGCCTTGTT |
| | | TGGATGTCTTCACAGGAAGACGCTTTTTTTGCGGCCG |
| | | CATACGCGTTGACATTGATTATTGACTAGTTATTAAT |
| | | AGTAATCAATTACGGGGTCATTAGTTCATAGCCCATA |
| | | TATGGAGTTCCGCGTTACATAACTTACGGTAAATGGC |
| | | CCGCCTGGCTGACCGCCCAACGACCCCCGCCCATTG |
| | | ACGTCAATAATGACGTATGTTCCCATAGTAACGCCAA |
| | | TAGGGACTTTCCATTGACGTCAATGGGTGGAGTATTT |
| | | ACGGTAAACTGCCCACTTGGCAGTACATCAAGTGTAT |
| | | CATATGCCAAGTACGCCCCCTATTGACGTCAATGACG |
| | | GTAAATGGCCCGCCTGGCATTATGCCCAGTACATGAC |
| | | CTTATGGGACTTTCCTACTTGGCAGTACATCTACGTA |
| | | TTAGTCATCGCTATTACCATGGTGATGCGGTTTTGGC |
| | | AGTACATCAATGGGCGTGGATAGCGGTTTGACTCAC |
| | | GGGGATTTCCAAGTCTCCACCCCATTGACGTCAATGG |
| | | GAGTTTGTTTTGGCACCAAAATCAACGGGACTTTCCA |
| | | AAATGTCGTAACAACTCCGCCCCATTGACGCAAATG |
| | | GGCGGTAGGCGTGTACGGTGGGAGGTCTATATAAGC |
| | | AGAGCTCGTTTAGTGAACCGTCAGATCGCCTGGAGA |
| | | CGCCATCCACGCTGTTTTGACCTCCATAGTAGACACC |
| | | GGGACCGATCCAGCCTCCGGACTCTAGAGGATCGAA |
| | | CCCTTAAGCCGCCACCATGGTGAGCAAGGGCGAGGA |
| | | GCTGTTCACCGGGGTGGTGCCCATCCTGGTCGAGCTG |
| | | GACGGCGACGTAAACGGCCACAAGTTCAGCGTGTCC |
| | | GGCGAGGGCGAGGGCGATGCCACCTACGGCAAGCTG |
| | | ACCCTGAAGTTCATCTGCACCACCGGCAAGCTGCCCG |
| | | TGCCCTGGCCCACCCTCGTGACCACCCTGACCTACGG |
| | | CGTGCAGTGCTTCAGCCGCTACCCCGACCACATGAA |
| | | GCAGCACGACTTCTTCAAGTCCGCCATGCCCGAAGG |
| | | CTACGTCCAGGAGCGCACCATCTTCTTCAAGGACGAC |
| | | GGCAACTACAAGACCCGCGCCGAGGTGAAGTTCGAG |
| | | GGCGACACCCTGGTGAACCGCATCGAGCTGAAGGGC |
| | | ATCGACTTCAAGGAGGACGGCAACATCCTGGGGCAC |
| | | AAGCTGGAGTACAACTACAACAGCCACAACGTCTAT |
| | | ATCATGGCCGACAAGCAGAAGAACGGCATCAAGGTG |
| | | AACTTCAAGATCCGCCACAACATCGAGGACGGCAGC |
| | | GTGCAGCTCGCCGACCACTACCAGCAGAACACCCCC |
| | | ATCGGCGACGGCCCCGTGCTGCTGCCCGACAACCAC |
| | | TACCTGAGCACCCAGTCCGCCCTGAGCAAAGACCCC |
| | | AACGAGAAGCGCGATCACATGGTCCTGCTGGAGTTC |
| | | GTGACCGCCGCCGGGATCACTCTCGGCATGGACGAG |
| | | CTGTACAAGTACTCAGATCTCGAGCTCAAGTGAACC |
| | | GGTCAGACATGATAAGATACATTGATGAGTTTGGAC |
| | | AAACCACAACTAGAATGCAGTGAAAAAAATGCTTTA |
| | | TTTGTGAAATTTGTGATGCTATTGCTTTATTTGTAACC |
| | | ATTATAAGCTGCAATAAACAAGTTAACAACAACAAT |
| | | TGCATTCATTTTATGTTTCAGGTTCAGGGGGAGGTGT |
| | | GGGAGGTTTTTTAAACACGTGCGGACCGAGCGGCCG |
| | | CAGGAACCCCTAGTGATGGAGTTGGCCACTCCCTCTC |
| | | TGCGCGCTCGCTCGCTCACTGAGGCCGGGCGACCAA |
| | | AGGTCGCCCGACGCCCGGGCTTTGCCCGGGCGGCCT |
| | | CAGTGAGCGAGCGAGCGCGCAGCTGCCTGCAGGCTT |
| | | GGATCCCAATGGCGCGCCGAGCTTGGCTCGAGCATG |
| | | GTCATAGCTGTTTCCTGTGTGAAATTGTTATCCGCTC |
| | | ACAATTCCACACAACATACGAGCCGGAAGCATAAAG |
| | | TGTAAAGCCTGGGGTGCCTAATGAGTGAGCTAACTC |
| | | ACATTAATTGCGTTGCGCTCACTGCCCGCTTTCCAGT |
| | | CGGGAAACCTGTCGTGCCAGCTGCATTAATGAATCG |
| | | GCCAACGCGCGGGGAGAGGCGGTTTGCGTATTGGGC |
| | | GCTCTTCCGCTTCCTCGCTCACTGACTCGCTGCGCTC |
| | | GGTCGTTCGGCTGCGGCGAGCGGTATCAGCTCACTCA |
| | | AAGGCGGTAATACGGTTATCCACAGAATCAGGGGAT |
| | | AACGCAGGAAAGAACATGTGAGCAAAAGGCCAGCA |
| | | AAAGGCCAGGAACCGTAAAAAGGCCGCGTTGCTGGC |
| | | GTTTTTCCATAGGCTCCGCCCCCCTGACGAGCATCAC |
| | | AAAAATCGACGCTCAAGTCAGAGGTGGCGAAACCCG |
| | | ACAGGACTATAAAGATACCAGGCGTTTCCCCCTGGA |
| | | AGCTCCCTCGTGCGCTCTCCTGTTCCGACCCTGCCGC |
| | | TTACCGGATACCTGTCCGCCTTTCTCCCTTCGGGAAG |
| | | CGTGGCGCTTTCTCATAGCTCACGCTGTAGGTATCTC |
| | | AGTTCGGTGTAGGTCGTTCGCTCCAAGCTGGGCTGTG |
| | | TGCACGAACCCCCCGTTCAGCCCGACCGCTGCGCCTT |
| | | ATCCGGTAACTATCGTCTTGAGTCCAACCCGGTAAGA |
| | | CACGACTTATCGCCACTGGCAGCAGCCACTGGTAAC |
| | | AGGATTAGCAGAGCGAGGTATGTAGGCGGTGCTACA |
| | | GAGTTCTTGAAGTGGTGGCCTAACTACGGCTACACTA |
| | | GAAGAACAGTATTTGGTATCTGCGCTCTGCTGAAGCC |
| | | AGTTACCTTCGGAAAAAGAGTTGGTAGCTCTTGATCC |
| | | GGCAAACAAACCACCGCTGGTAGCGGTGGTTTTTTTG |
| | | TTTGCAAGCAGCAGATTACGCGCAGAAAAAAAGGAT |
| | | CTCAAGAAGATCCTTTGATCTTTTCTACGGGGTCTGA |
| | | CGCTCAGTGGAACGAAAACTCACGTTAAGGGATTTT |
| | | GGTCATGAGATTATCAAAAAGGATCTTCACCTAGATC |
| | | CTTTTAAATTAAAAATGAAGTTTTAAATCAATCTAAA |
| | | GTATATATGAGTAAACTTGGTCTGACAGTTAGAAAA |
| | | ACTCATCGAGCATCAAATGAAACTGCAATTTATTCAT |
| | | ATCAGGATTATCAATACCATATTTTTGAAAAAGCCGT |
| | | TTCTGTAATGAAGGAGAAAACTCACCGAGGCAGTTC |
| | | CATAGGATGGCAAGATCCTGGTATCGGTCTGCGATTC |
| | | CGACTCGTCCAACATCAATACAACCTATTAATTTCCC |
| | | CTCGTCAAAAATAAGGTTATCAAGTGAGAAATCACC |
| | | ATGAGTGACGACTGAATCCGGTGAGAATGGCAAAAG |
| | | TTTATGCATTTCTTTCCAGACTTGTTCAACAGGCCAG |
| | | CCATTACGCTCGTCATCAAAATCACTCGCATCAACCA |
| | | AACCGTTATTCATTCGTGATTGCGCCTGAGCGAGACG |
| | | AAATACGCGATCGCTGTTAAAAGGACAATTACAAAC |
| | | AGGAATCGAATGCAACCGGCGCAGGAACACTGCCAG |
| | | CGCATCAACAATATTTTCACCTGAATCAGGATATTCT |
| | | TCTAATACCTGGAATGCTGTTTTCCCAGGGATCGCAG |
| | | TGGTGAGTAACCATGCATCATCAGGAGTACGGATAA |
| | | AATGCTTGATGGTCGGAAGAGGCATAAATTCCGTCA |
| | | GCCAGTTTAGTCTGACCATCTCATCTGTAACATCATT |
| | | GGCAACGCTACCTTTGCCATGTTTCAGAAACAACTCT |
| | | GGCGCATCGGGCTTCCCATACAATCGATAGATTGTCG |
| | | CACCTGATTGCCCGACATTATCGCGAGCCCATTTATA |
| | | CCCATATAAATCAGCATCCATGTTGGAATTTAATCGC |
| | | GGCCTAGAGCAAGACGTTTCCCGTTGAATATGGCTCA |
| | | TACTCTTCCTTTTTCAATATTATTGAAGCATTTATCAG |
| | | GGTTATTGTCTCATGAGCGGATACATATTTGAATGTA |
| | | TTTAGAAAAATAAACAAATAGGGGTTCCGCGCACAT |
| | | TTCCCCGAAAAGTGCCACCTGACGTCTAAGAAACCA |
| | | TTATTATCATGACATTAACCTATAAAAATAGGCGTAT |
| | | CACGAGGCCCTTTCGTC |

### Example 6: Multiplexed Targeting of LRRK2 and SNCA

Because polymorphisms in either LRRK2 (G2019S) or SNCA are associated with increased risk of idiopathic Parkinson's Disease, simultaneous manipulation of the LRRK2 and SNCA mRNA can be a useful treatment. RNA editing, as illustrated in the current disclosure, is modular; the RNA editing enzyme and the RNA targeting engineered polynucleotides (e.g., gRNA) are two different entities. Therefore, RNA editing can be multiplexed to correct multiple distinct target RNAs simultaneously. For example, to treat idiopathic Parkinson's Disease patients with contributing polymorphisms in LRRK2 (G2019S) and SNCA, two different engineered polynucleotide sequences are generated. The first engineered polynucleotide can comprise the sequence of an engineered polynucleotide (guide RNA) listed in Table 13. These first engineered polynucleotides (e.g., guide RNAs) can edit the mRNA to generate a modified polypeptide that comprises G2019S (G>A conversion at the 6055^{th} nucleotide) when translated, as illustrated in Example 3. The second engineered polynucleotide (guide RNA) can target the start ATG of SNCA. It can convert any nucleotide of the start A of ATG into G. Since the start ATG is removed, the expression of SNCA should decrease. These two engineered polynucleotide sequences can comprise an upstream U7, U1, U6, H1, 7SK promoter. A DNA sequence encoding the two engineered polynucleotides can be cloned into a single viral vector—such as an adenoviral vector, an adeno-associated viral vector (AAV), a lentiviral vector, or a retroviral vector—that transcribes these DNA sequences to produce the engineered polynucleotide sequences. The vector is injected into the brain of the patient by intracerebroventricular injection.

The RNA editing of LRRK2 is monitored as follows: ˜1×10\hat{ }5 are collected for RNA isolation after a week. At collection, cells are spun at 1,500×g for 1 min. The media are removed. 180 ul of RLT buffer+BMe is added to each well. Qiagen RNeasy protocol and kit are used to isolate the RNAs from the cell. New England Biolabs (NEB) ProtoScript II First-Strand cDNA synthesis kit is used to synthesize cDNA from the isolated RNA. cDNA of LRRK2 is sequenced by the Sanger sequencing. Sanger traces are analyzed to assess the editing efficiency of each IVT guide. The RNA editing efficiency, the A>G conversion in LRRK2 mRNA, is calculated by the difference of trace signal of the LRRK2 mRNA with a G (edited) and an A (unedited) at the 6055^{th} nucleotide.

The expression level of SNCA is monitored as follows: knockdown of SNCA protein is assessed using Western Blot, ELISA, and Meso Scale Discovery (MSD) analysis of SNCA protein levels.

### Example 7: Editing RAB7A and SNCA mRNA

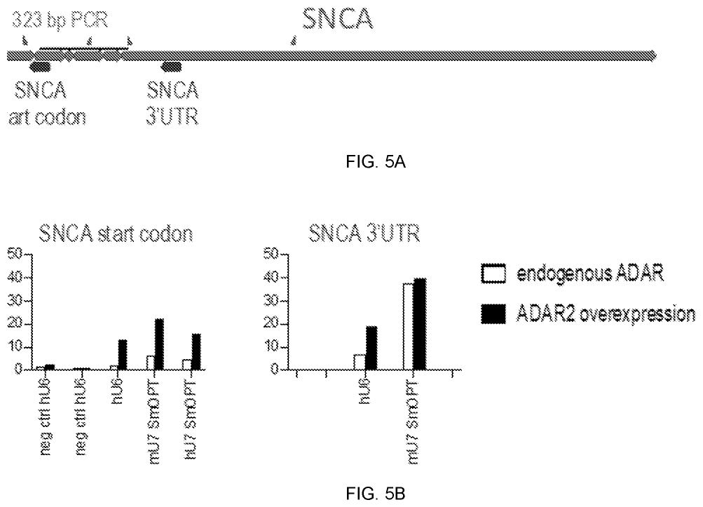

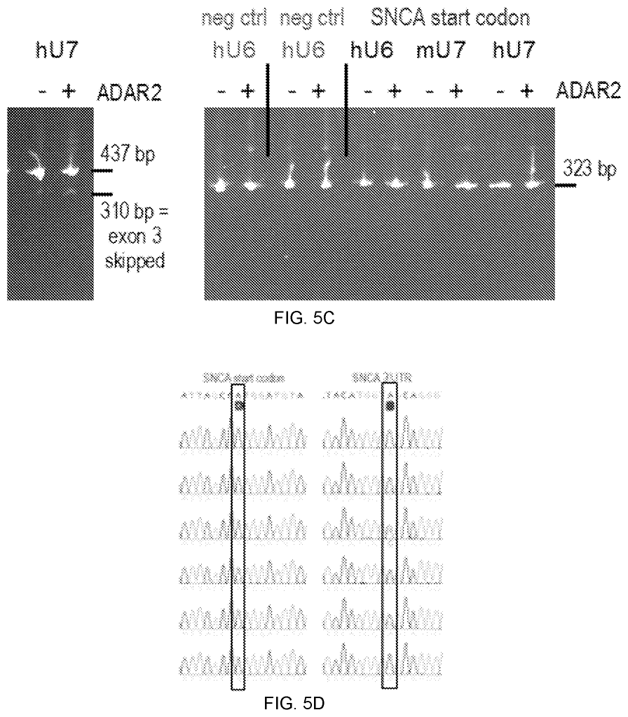

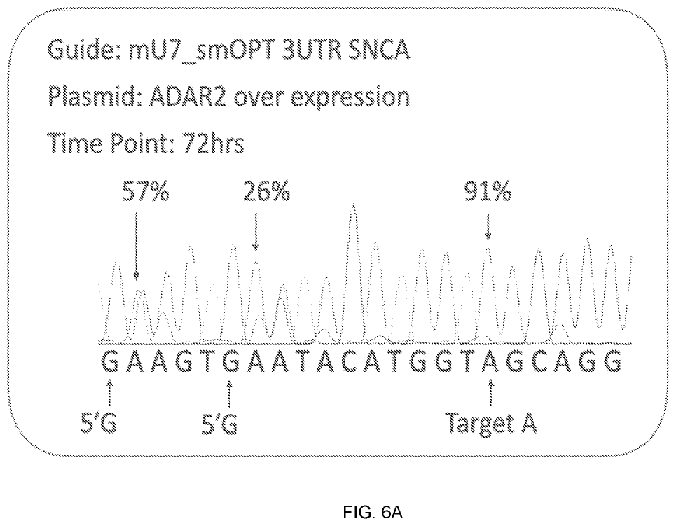

In this example, different regions of RAB7a and alpha-synuclein (SNCA) were edited using different engineered polynucleotides (e.g., guide RNA constructs). For RAB7a, exons 1, 2, and the 3′ UTR were targeted for editing, whereas the start codon and the 3′ UTR of SNCA were targeted. FIG. 5A shows a schematic of the exon structure of human SNCA. Exons are shown as segments; the coding region is denoted as a black line above. Locations of the guide RNA targeting sites are shown as arrows; PCR primers are shown at the top.

100 nt engineered polynucleotides (e.g., guide RNAs) targeting human RAB7A exon 1, exon 3, or 3′UTR, or human SNCA start codon or 3′UTR were expressed using the hU6 promoter without a 3′ hairpin or the mU7 or hU7 promoters with a 3′ SmOPT U7 hairpin. FIG. 5B summarizes the results of the SNCA editing using the different engineered polynucleotides (e.g., guide RNA constructs) in the presence or absence of ADAR2 overexpression. U7 promoters combined with a 3′ SmOPT U7 hairpin enhanced ADAR editing at each target site (measured by Sanger sequencing). While constructs targeting the 3′UTRs worked equally well under endogenous versus overexpressed ADAR levels, constructs targeting other areas still benefited from ADAR2 overexpression.

To confirm whether differential exon selection occurred, cDNA derived from the edited transcripts were isolated and PCR amplified using the denoted primers. The RAB7a primers, which span the coding determining sequence of RAB7a, generate a 437 bp amplicon if the exon structure is maintained. If exon 3 of RAB7a is skipped, a 310 bp amplicon is expected. Using the SNCA primers, a 323 bp PCR amplicon is expected. FIG. 5C shows minimal exon 3 skipping for RAB7a and the presence of significant SNCA amplicon.

FIG. 5D shows Sanger sequencing chromatograms showing specific editing at the target adenosine of the SNCA transcripts. The box indicates the on-target editing site.

### Example 8: On and Off Target Editing of the 3′ UTR of SNCA in K562 Cells

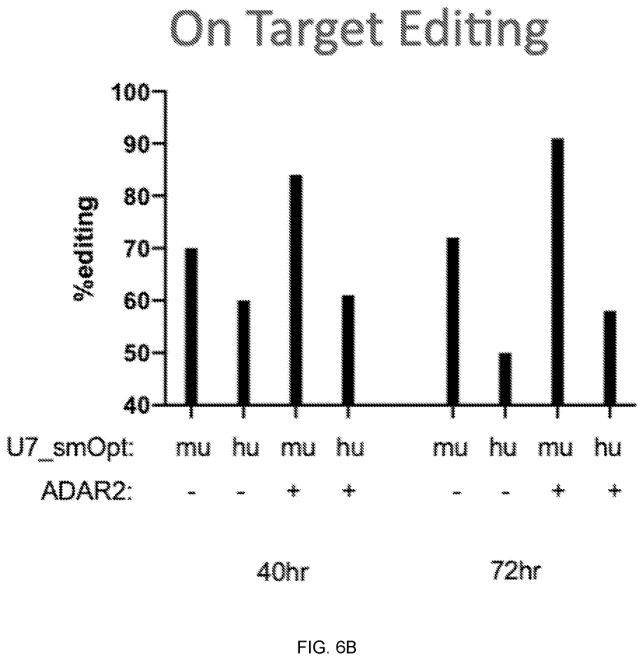

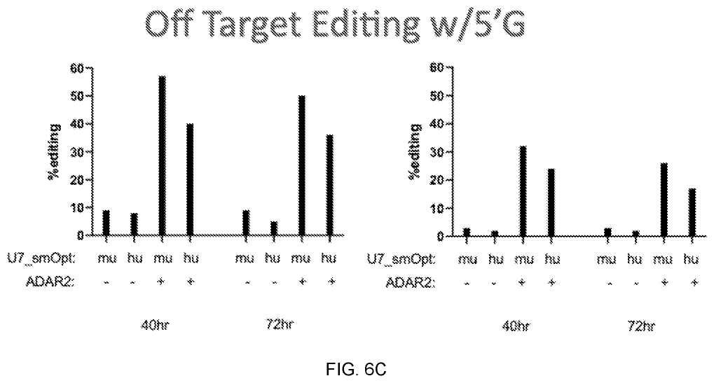

Disclosed herein are compositions of engineered polynucleotides (e.g., guide RNAs) under a U7 promoter and also comprising a smOPT hairpin sequence. Said engineered polynucleotides (e.g., guide RNAs) can hybridize to a region of a target RNA sequence corresponding to SNCA, to facilitate ADAR-mediated editing of an adenosine (see FIG. 6A-FIG. 6C). Editing of the SNCA transcript was assessed by transfection of the engineered polynucleotides (e.g., guide RNAs) in K562 cells which overexpress SNCA. 1.5 μg of the engineered polynucleotides (e.g., guide RNAs) was transfected into 2×10⁵ SNCA-overexpressing K562 cells via nucleofection (Lonza). RNA editing was measured 40 and 72 hours after transfection. FIG. 6A is a Sanger sequence chromatogram showing on target editing (as denoted by “Target A”) of 91%. FIG. 6B is a graph showing on target editing at the 40 hour and 72 hour timepoints in K562 cells with and without ADAR2 under either a mouse U7 promoter or a human U7 promoter. High levels of editing (greater than 40% for all constructs) over a sustained period of time were observed. FIG. 6C depicts graphs showing off-target editing of adenosines having a G directly 5′ of the off-target A.

### Example 9: Optimized Engineered Polynucleotides for Editing LRRK2

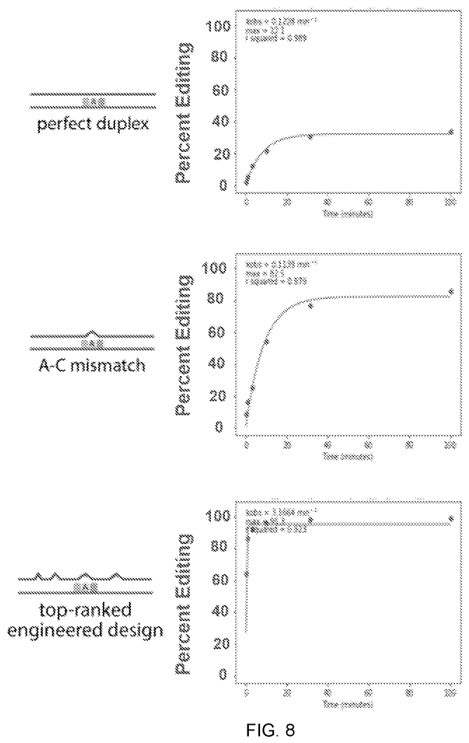

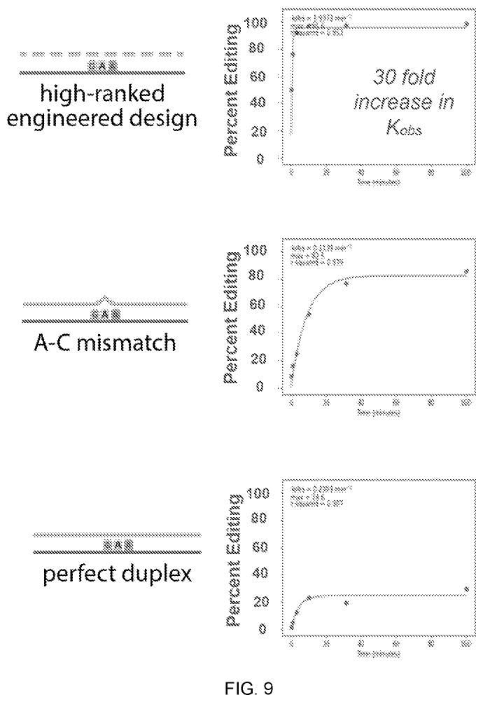

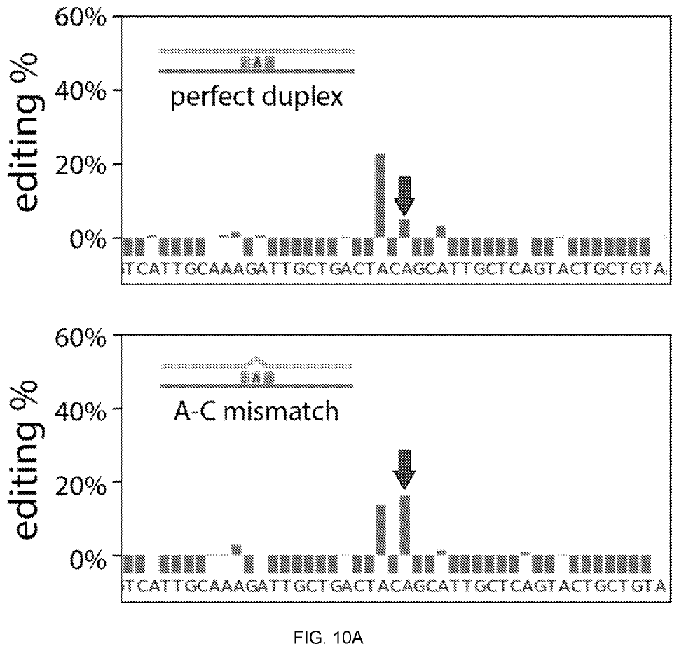

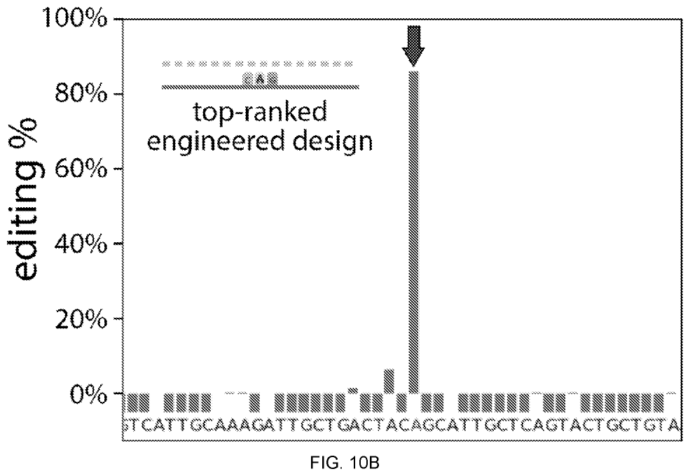

Disclosed herein are optimized engineered polynucleotides (e.g., guide RNAs) for editing LRRK2. FIG. 8 and FIG. 9 show the editing kinetics of two optimized top-ranked engineered polynucleotides (e.g., guide RNAs), as compared to that of an engineered polynucleotide (e.g., guide RNA) with a perfect duplex or an engineered polynucleotide (e.g., guide RNA) with a single A-C mismatch. The top ranked engineered polynucleotide (e.g., guide RNA) had 30-fold increase in K_{obs} compared to those of the other engineered polynucleotides (e.g., guide RNAs) designs. FIG. 10B shows the editing frequency of an optimized top-ranked engineered polynucleotide (e.g., guide RNA), as compared to that of an engineered polynucleotide (e.g., guide RNA) with a perfect duplex or an engineered polynucleotide (e.g., guide RNA) with a single A-C mismatch shown in FIG. 10A. The on-target target base editing of the optimized top-ranked engineered polynucleotide (e.g., guide RNA) is more than 80%, while the engineered polynucleotide (e.g., guide RNA) with a perfect duplex or the engineered polynucleotide (e.g., guide RNA) with a single A-C mismatch provided only less than 20% on-target editing.

### Example 10: Engineered Polynucleotides with SmOPT and a U7 Hairpin and Targeting LRRK2 mRNA

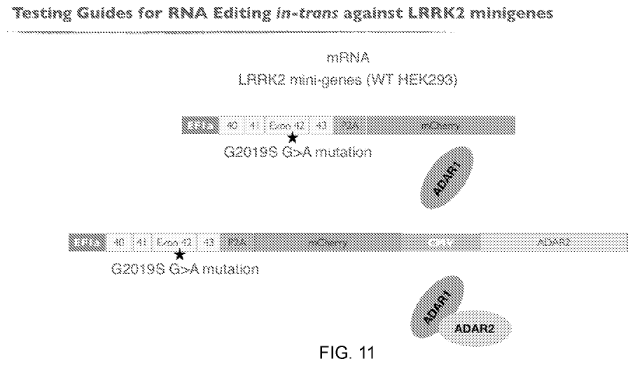

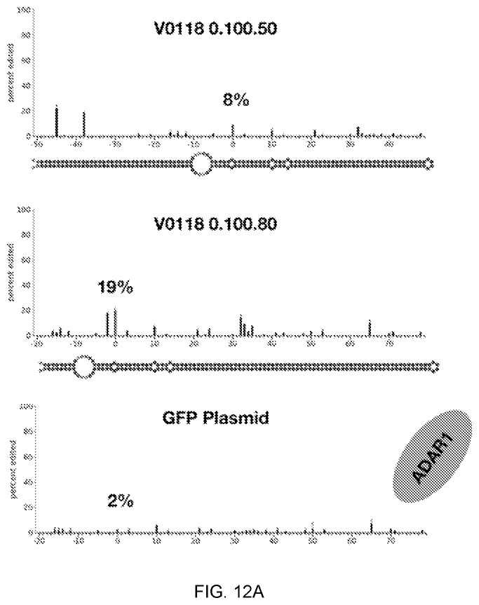

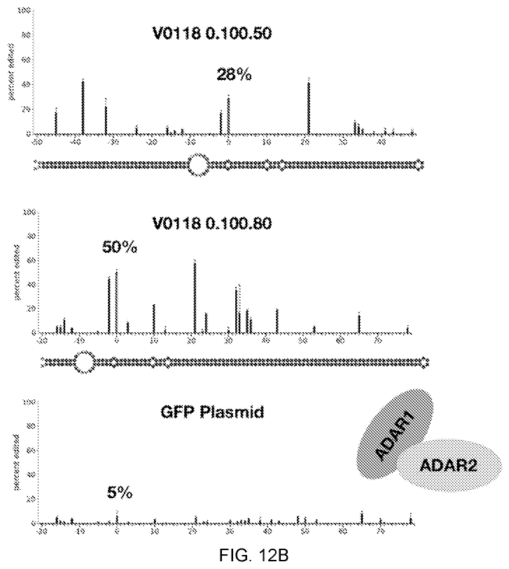

This example describes optimized engineered polynucleotides (e.g., guide RNAs) of the present disclosure under the control of a U1 promoter, an SmOPT sequence, and a U7 hairpin, wherein the engineered polynucleotide (e.g., guide RNA) is designed to target LRRK2. Two engineered polynucleotides (e.g., guide RNAs) designs targeting the nucleotide encoding the LRRK2 G2019S mutation were tested, both with SmOPT and a U7 hairpin. The first guide (“V0118 0.100.50”) contained 100 bases with the target A in LRRK2 mRNA to be edited across from the base at position 50 in the guide. The second guide (“V0118 0.100.80”) contained 100 bases with the target A in LRRK2 to be edited across from the base at position 80 in the guide. Engineered polynucleotides (e.g., guide RNAs) were tested for their ability to facilitate ADAR-mediated RNA editing of a nucleotide of a codon encoding the G2019S LRRK2 mutation in WT HEK293 cells transfected with a piggyBac vector carrying a LRRK2 minigene. WT HEK293 cells naturally express ADAR1. In experiments in which RNA editing mediated via ADAR1 and ADAR2 was tested, ADAR2 was overexpressed in cells via the same piggyBac vector carrying the LRRK2 minigene. Schematics of the piggyBac constructs are shown in FIG. 11. Experiments were conducted in the presence of ADAR1 only (FIG. 12A) or ADAR1 and ADAR2 (FIG. 12B). A GFP plasmid was used as a control. FIG. 12A and FIG. 12B show that engineered polynucleotides (e.g., guide RNAs) containing SmOPT and a U7 hairpin facilitated an on-target editing efficiency of 8% in the presence of ADAR1 only and 28% in the presence of ADAR1 and ADAR2. FIG. 12A and FIG. 12B show that engineered polynucleotides (e.g., guide RNAs) containing SmOPT and a U7 hairpin facilitated an on-target editing efficiency of 19% in the presence of ADAR1 only and 58% in the presence of ADAR1 and ADAR2. Further experimentation demonstrated that the first engineered polynucleotide (e.g., first guide RNA) (“V0118 0.100.50”) had a Gibbs free energy (delta G) of −161.98 kcal/mol and the second engineered polynucleotide (e.g., second guide RNA) (“V0118 0.100.80”) had a delta G of −169.44 kcal/mol. The structures of both engineered polynucleotides (e.g., guide RNAs) are shown beneath the graphs in FIG. 12A-FIG. 12B. As seen in the structural diagrams, the second engineered polynucleotide (e.g., second guide RNA) (“V0118 0.100.50”) formed a longer continuous stretch of duplex RNA with the target region of the target RNA. Sequences tested are shown below in Table 16.

TABLE 16
Exemplary LRRK2 Engineered Polynucleotide (e.g., Guide RNAs)

| SEQ ID NO | Engineered Polynucleotide Sequence |
|---|---|
| V0118 0.100.50 | GTGCCCTCTGATGTTTTTATCCCCATTCTACAGCATTA |
| (SEQ ID NO: 81) | CGGAGCAGTGCCGTAGTGTCGTTTCTTTGCAATGATG |
| | GCAGCATTGGATACAGTGTGAAAAgtggAATTTTTGGA |
| | GCAGGTTTTCTGACTTCGGTCGGAAAACCCCT |
| V0118 0.100.80 | CTGGCAACTTCAGGTGCACGAAACCCTGGTGTGCCCT |
| (SEQ ID NO: 82) | CTGATGTTTTTATCCCCATTCTACAGCATTACGGAGCA |
| | GTGCCGTAGTGTCGTTTCTTTGCAAgtggAATTTTTGGA |
| | GCAGGTTTTCTGACTTCGGTCGGAAAACCCCT |

### Example 11: LRRK2 Editing with Circular Engineered Polynucleotides

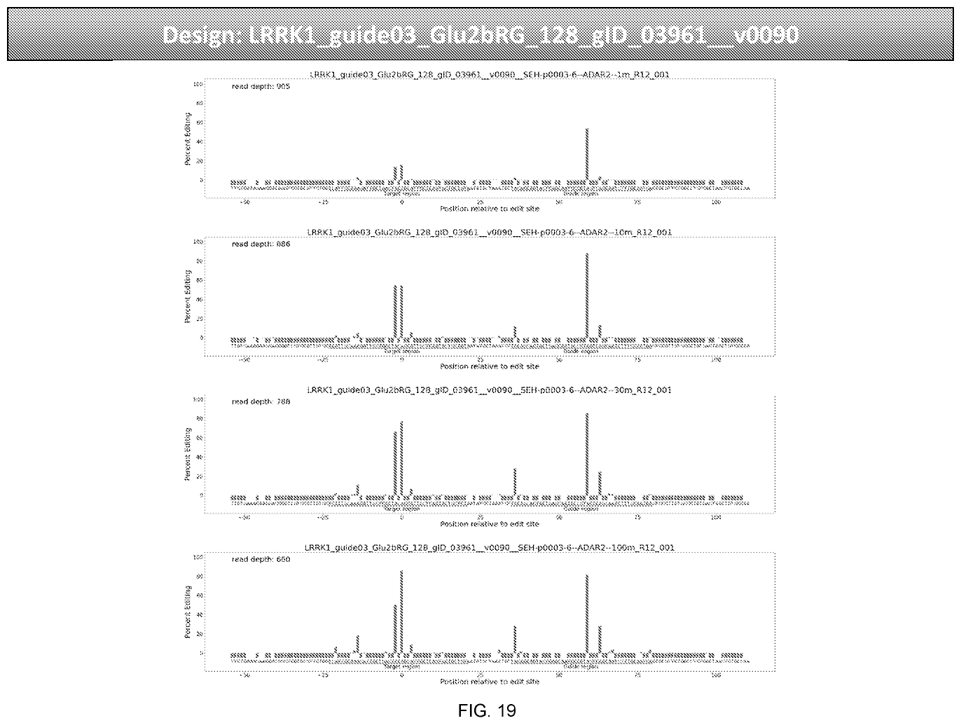

This example describes LRRK2 editing with circular engineered polynucleotides of the present disclosure. 20,000 cells were transfected with 500 ng of plasmid encoding for circular engineered polynucleotides. Cells tested include HEK293 cells, which express endogenous ADAR1, transfected with a piggyBac vector carrying the LRRK2 minigene carrying the nucleotide mutation encoding the G2019S mutation (corresponding data in FIG. 19, at top) and HEK293 cells, which express endogenous ADAR1, transfected with a piggyBac vector carrying ADAR2 and the LRRK2 minigene carrying the nucleotide mutation encoding the G2019S mutation.

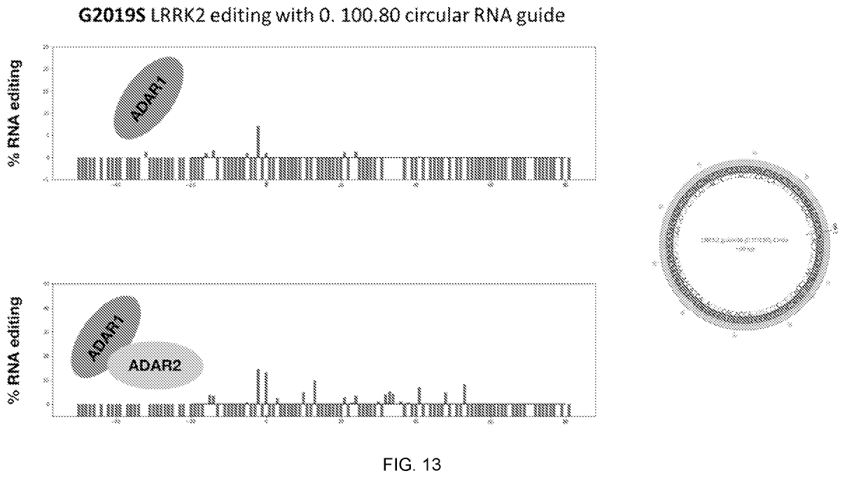

RNA was isolated 48 hours post transfection and converted into a cDNA library. PCR and Sanger sequencing analyses were carried out. Percent editing was determined using the EditR program. The circular engineered polynucleotide was 100 bases long with the A/C mismatch engineered 80 bases from the 5′ end of the guide. The circular engineered polynucleotide (SEQ ID NO: 83) comprises a sequence of 5′-CTGGCAACTTCAGGTGCACGAAACCCTGGTGTGCCCTCTGATGTTCTTATCCCCATTC TACAGCAGTACTGAGCAATGCCGTAGTCAGCAATCTTTGCAA-3′. FIG. 13 shows graphs of on-target and off-target ADAR1 editing and ADAR1+ADAR2 editing of the nucleotide of the codon encoding the G2019S mutation.

### Example 12: In Vitro Editing of LRRK2 mRNA in iPSC-Derived LRRK2-G2019S Dopaminergic Neurons

This example describes editing in vitro in induced pluripotent stem cell (iPSC)-derived neurons that can express LRRK2-G2019S mutant protein. Culturing, induction, maturation, and transfection or transduction of iPSC neurons are optimized for screening of editing by engineered polynucleotides. Each dopaminergic neuronal phenotype is characterized and validated via TaqMan qPCR, flow cytometry, and immunofluorescence. iPSCs, neural stem cells (NSCs), neural progenitor cell (NPCs), or derived neuronal cells are then transfected or transduced with engineered polynucleotides targeting a nucleotide of the codon encoding the LRRK2-G2019S mutation, and editing efficiency is quantified using Sanger sequencing, ddPCR, and amplicon next generation sequencing of the sequence encoding the LRRK2-G2019S locus. In vitro biochemical editing of LRRK2 mRNA that is translated into LRRK2 protein is assessed using LC-MS/MS of the LRRK2 protein, and Western Blot and Meso Scale Discovery (MSD) analysis of LRRK2 substrates (e.g., phospho-Rab (-8,-10,-35), LRRK2 autophosphorylation).

### Example 13: Ex Vivo Editing of LRRK2 mRNA in Primary Cortical Neurons of hLRRK2-G2019S Mice

This example describes editing of the nucleotide of the codon encoding the LRRK2-G2019S mutation ex vivo in primary cortical neurons isolated from hLRRK2-G2019S mice. Primary microdissection and culturing of the primary neurons from the hLRRK2-G2019S mice are optimized prior to transfection or transduction of the engineered polynucleotides. The isolated cortical neurons are then transfected with engineered polynucleotides targeting the mRNA encoding the LRRK2-G2019S mutation, and editing efficiency is quantified using Sanger sequencing, ddPCR, and amplicon next generation sequencing of the LRRK2-G2019S encoding locus. Further, the ex vivo biochemical editing of LRRK2 mRNA that is translated into LRRK2 protein is assessed using LC-MS/MS of the LRRK2 protein, Western Blot and MSD analysis of LRRK2 substrates (e.g., phospho-Rab (-8,-10,-35), LRRK2 autophosphorylation).

### Example 14: In Vivo Editing of LRRK2 mRNA in hLRRK2-G2019S Mice

This example describes editing of mRNA encoding LRRK2-G2019S in vivo in hLRRK2-G2019S mice. Engineered polynucleotides targeting mRNA encoding the LRRK2-G2019S mutation are administered to the brain of the hLRRK2-G2019S mice via intracerebroventricular, intraparenchymal, intracisternal, or intrathecal injection. Brain tissue is then isolated from the hLRRK2-G2019S mice and processed to isolate LRRK2 nucleic acid and protein. Editing efficiency is quantified using ddPCR and amplicon next generation sequencing of the locus encoding LRRK2-G2019S. Further, the in vivo biochemical editing of LRRK2 mRNA that is translated into LRRK2 protein is assessed using LC-MS/MS of the LRRK2 protein, Western Blot and MSD analysis of LRRK2 substrates (e.g., phospho-Rab (-8,-10,-35), LRRK2 autophosphorylation).

### Example 15: In Vitro Editing of SNCA mRNA in LUHMES and iPSC-Derived Dopaminergic Neurons

This example describes SNCA editing in vitro in LUHMES and iPSC-derived dopaminergic neurons. Culturing, induction, differentiation, and transfection and transduction of LUHMES and iPSC-derived neurons are optimized for screening of editing by engineered polynucleotides. Each dopaminergic neuronal phenotype is characterized and validated via TaqMan qPCR, flow cytometry, and immunofluorescence. Neurons are then transfected or transduced with engineered polynucleotides targeting SNCA, and editing efficiency is quantified using qPCR, ddPCR and amplicon next generation sequencing of the SNCA locus. The in vitro biochemical knockdown of SNCA protein is assessed using Western Blot, ELISA, and MSD analysis of SNCA protein levels.

### Example 16: Ex Vivo Editing of SNCA mRNA in Primary Cortical Neurons of hSNCA Mice

This example describes SNCA mRNA editing and SNCA protein knockdown ex vivo in primary cortical neurons isolated from hSNCA mice. Primary microdissection and culturing of the primary neurons from the SNCA mice are optimized prior to transfection or transduction of the engineered polynucleotides. The isolated cortical neurons are then transfected or transduced with engineered polynucleotides targeting SNCA, and editing efficiency is quantified using qPCR<ddPCR, and amplicon next generation sequencing of the SNCA locus. Further, the ex vivo biochemical knockdown of SNCA protein is assessed using Western Blot, ELISA, and MSD analysis of SNCA protein levels.

### Example 17: In Vivo Editing of SNCA mRNA in hSNCA Mice

This example describes SNCA editing in vivo in hSNCA mice. Engineered polynucleotides targeting SNCA are administered to the brain of the hSNCA mice via intracerebroventricular, intraparenchymal, intracisternal, or intrathecal injection. Brain tissue is then isolated from the hSNCA mice and processed to isolate SNCA nucleic acid and protein. Editing efficiency is quantified using qPCR, ddPCR, and amplicon next generation sequencing of the SNCA locus. Further, the in vivo biochemical knockdown of SNCA protein is assessed from the isolated SNCA protein using Western Blot, ELISA, and MSD analysis of SNCA protein levels.

### Example 18: Engineered Polynucleotides Comprising Targeting LRRK2 mRNA

This example describes engineered polynucleotides targeting LRRK2 mRNA. The region of the LRRK2 mRNA that was targeted was an A at position 6055 of a LRRK2 mRNA, which encodes for a pathogenic G2019S mutant protein.

Self-annealing RNA structures comprising the engineered polynucleotide sequences of Table 17 (and control engineered polynucleotide sequences) and the sequences of the regions targeted by the engineered polynucleotides were contacted with an RNA editing entity (e.g., a recombinant ADAR1 and/or ADAR2) under conditions that allow for the editing of the regions targeted by the engineered polynucleotides. The regions targeted by the engineered polynucleotides were subsequently assessed for editing using next generation sequencing (NGS).

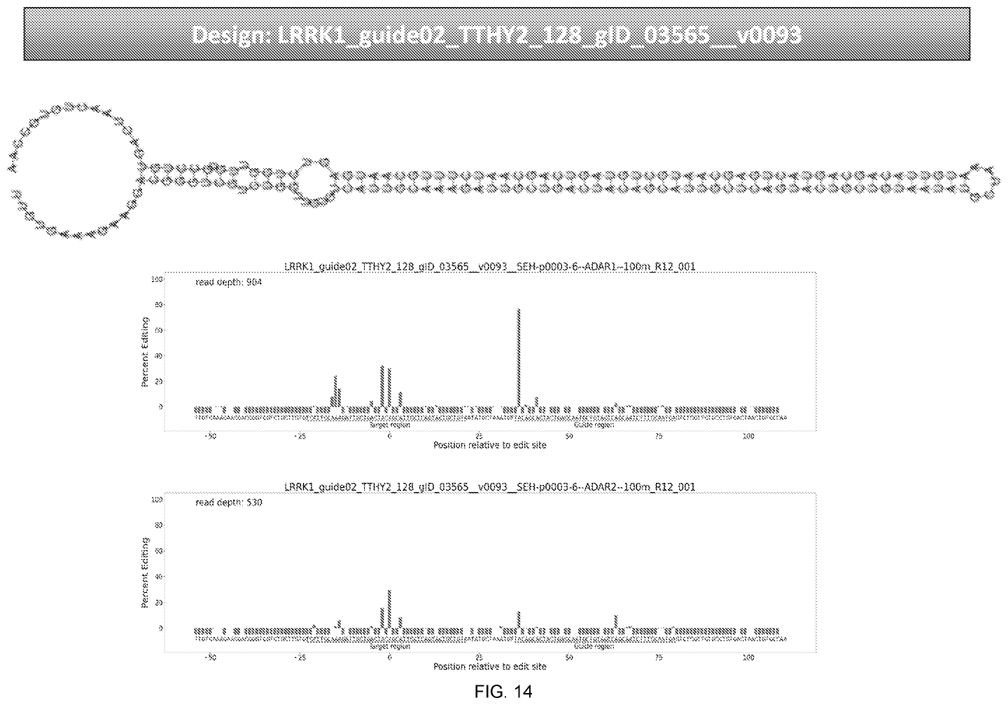

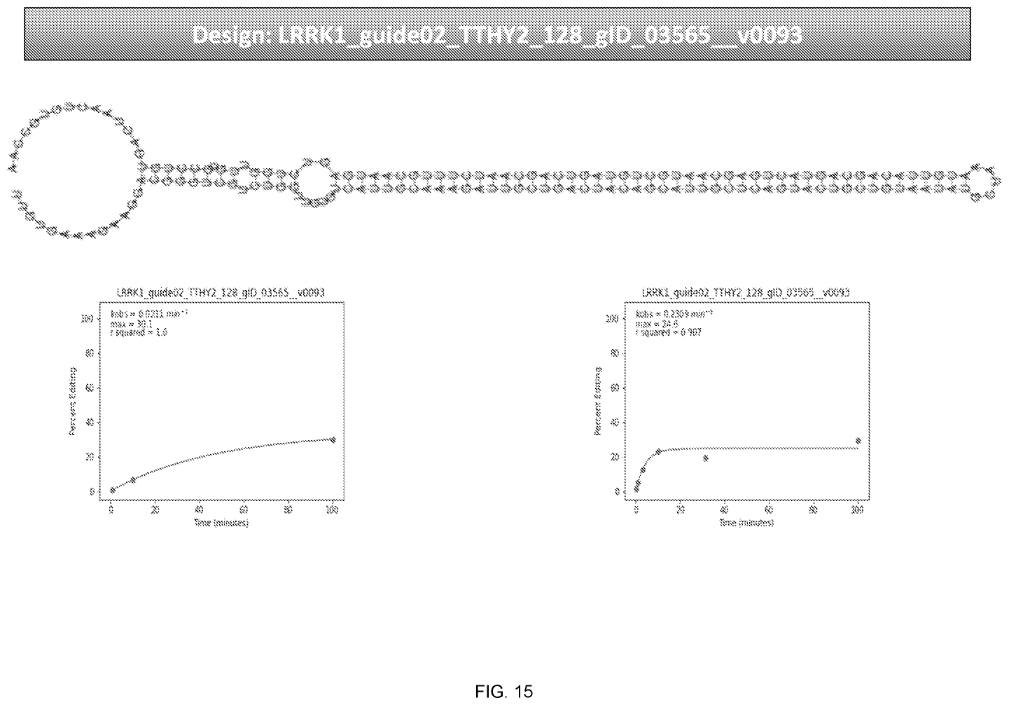

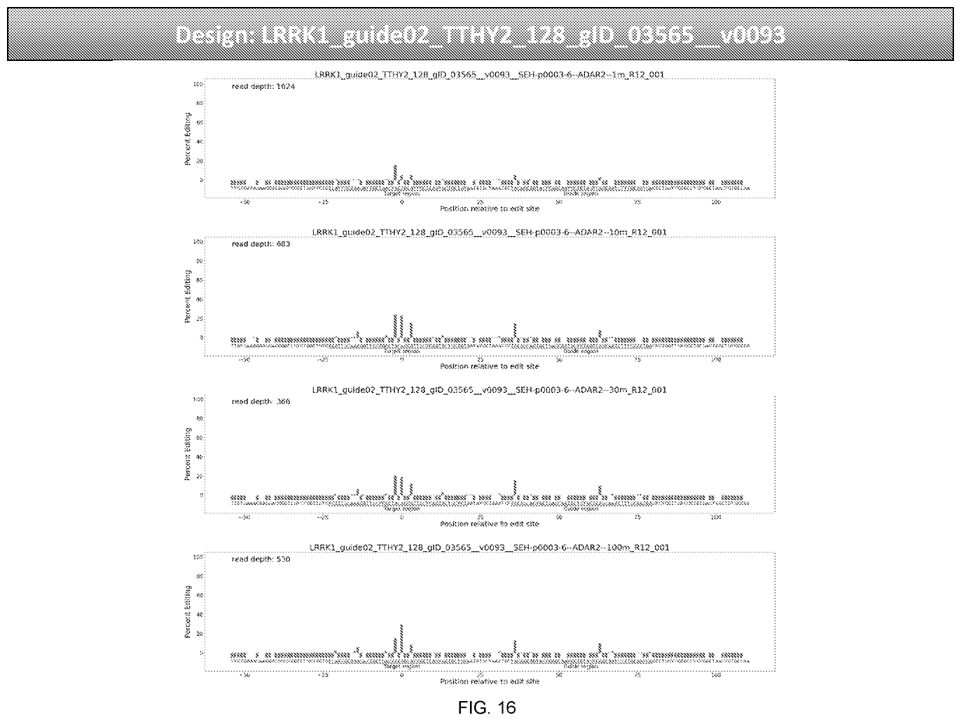

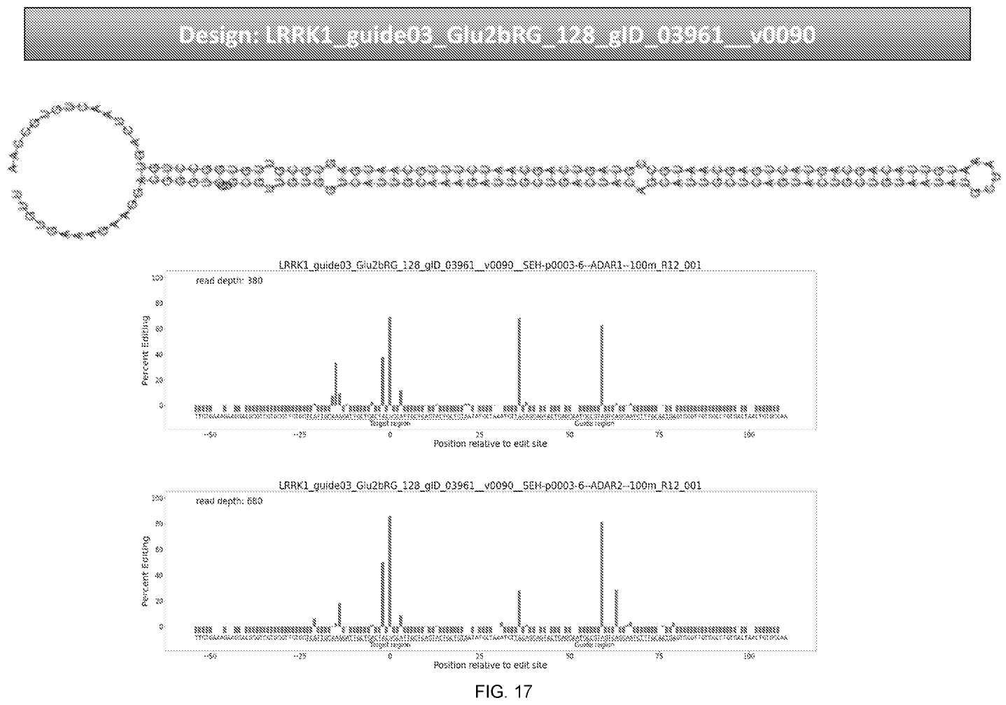

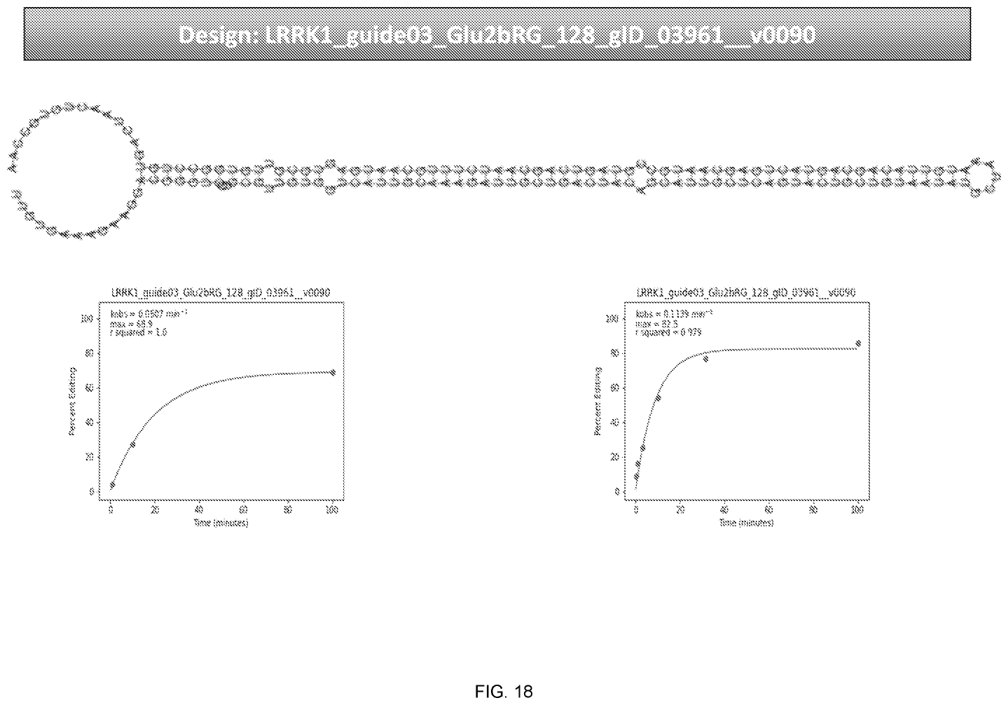

FIG. 14-FIG. 19 show control engineered polynucleotide designs for targeting LRRK2, the percentage editing as a function of time for each engineered polynucleotide as determined by sequencing, and the editing at the target A to be edited (“0” on the x-axis) and at RNA editing at off-target positions (represented as black bars at positions that are not “0”).

LRRK1_guide02_TTHY2_128_gID_03565_v0093 is
(SEQ ID NO: 84)
5’-TACAGCAGTACTGAGCAATGCTGTAGTCAGCAATCTTTGCAATGA-
3’.
LRRK1_guide03_Glu2bRG_128_gID_03961_v0090 is
(SEQ ID NO: 85)
5’-TACAGCAGTACTGAGCAATGCCGTAGTCAGCAATCTTTGCAATGA-
3’.

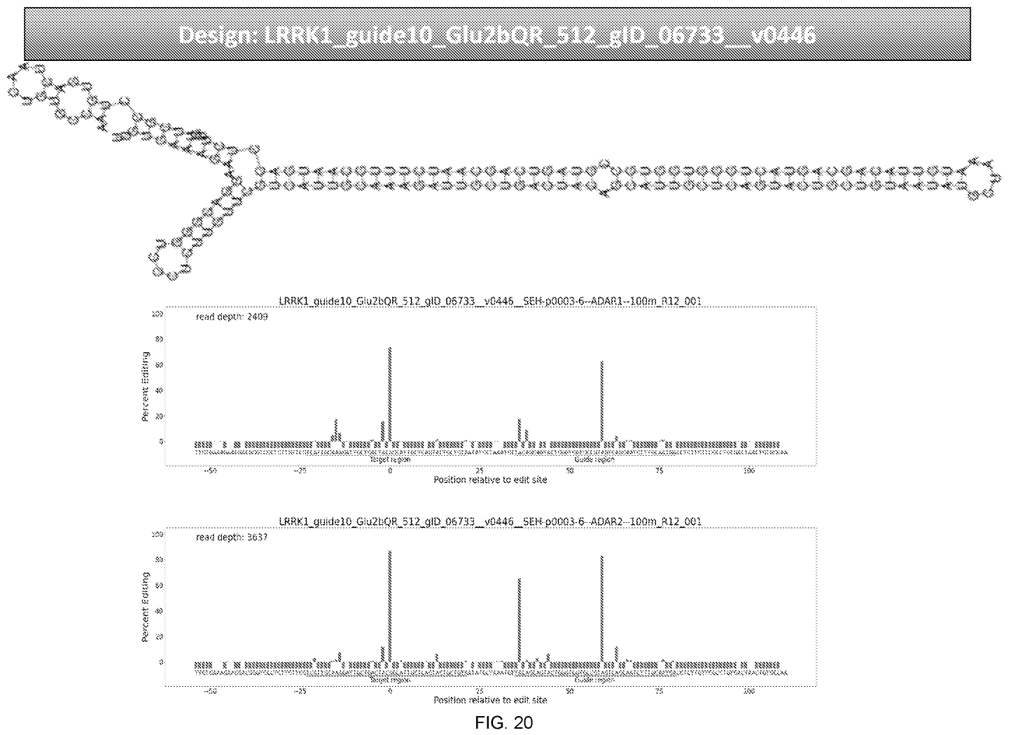

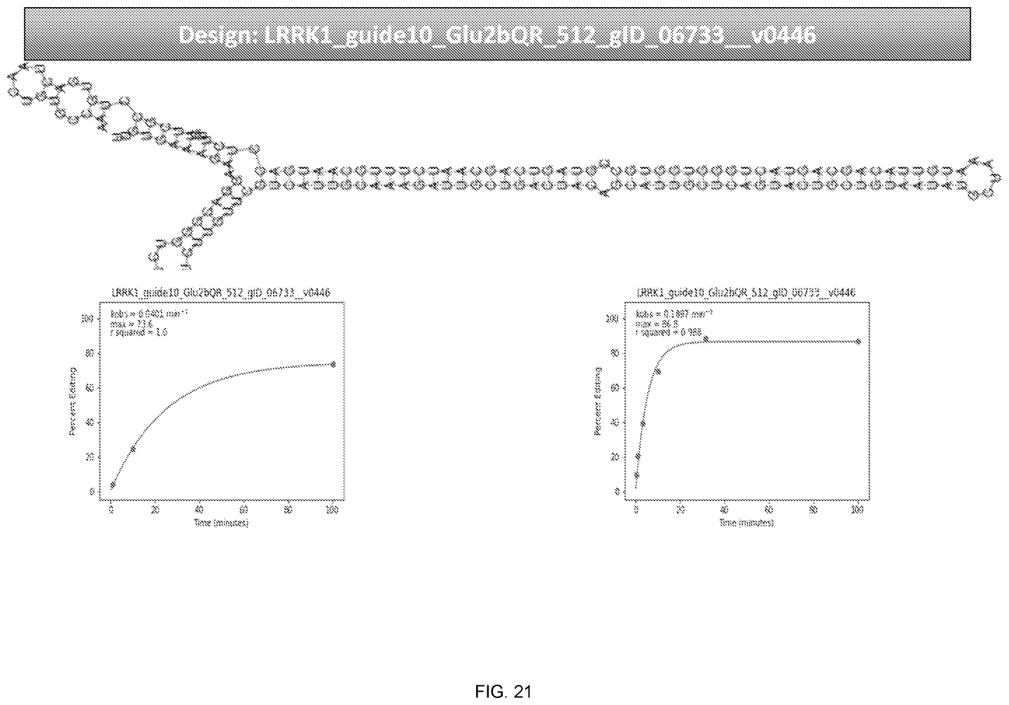

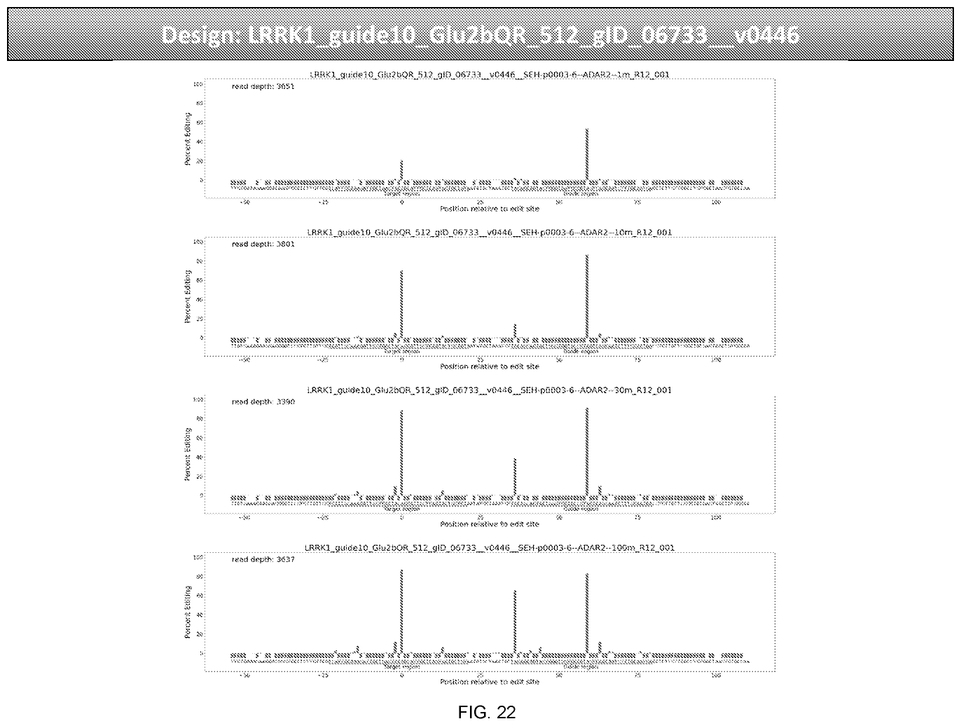

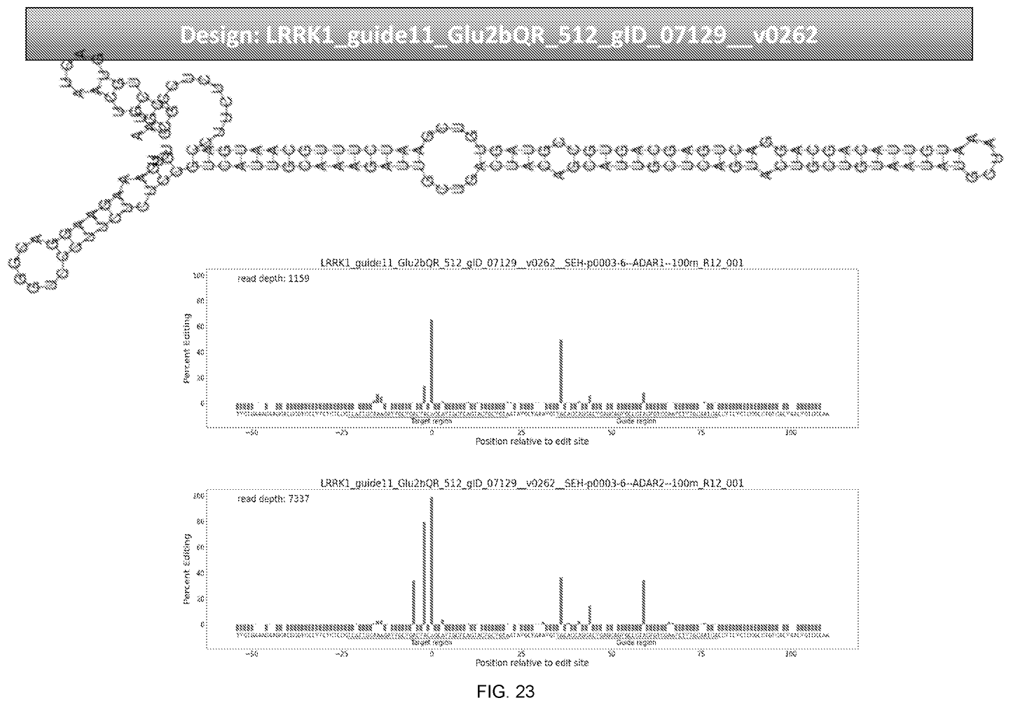

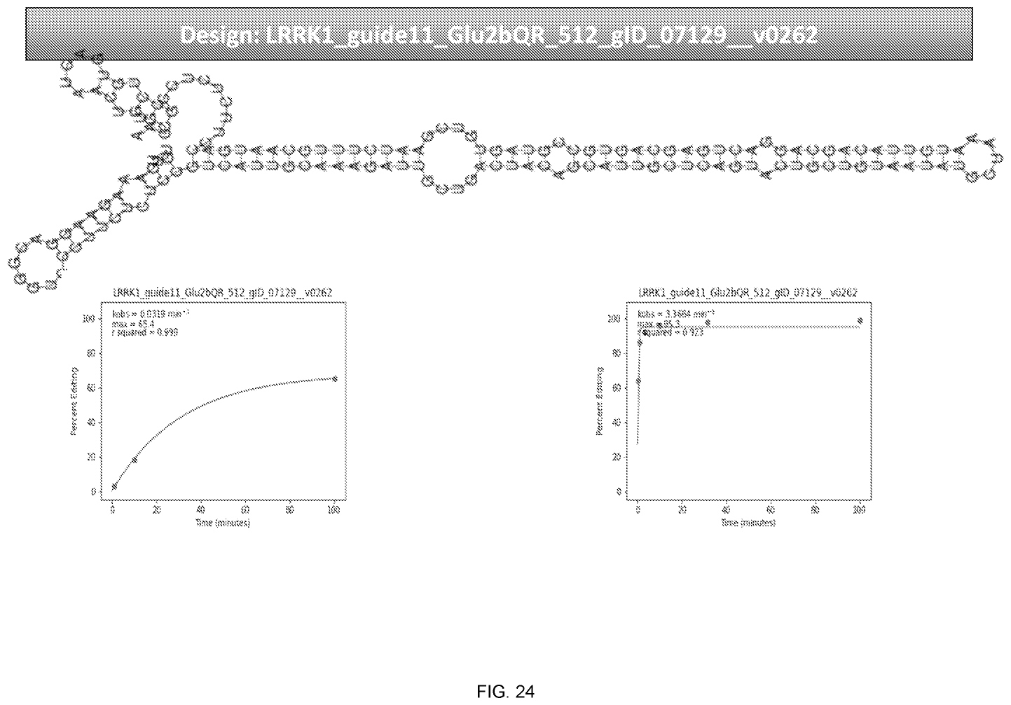

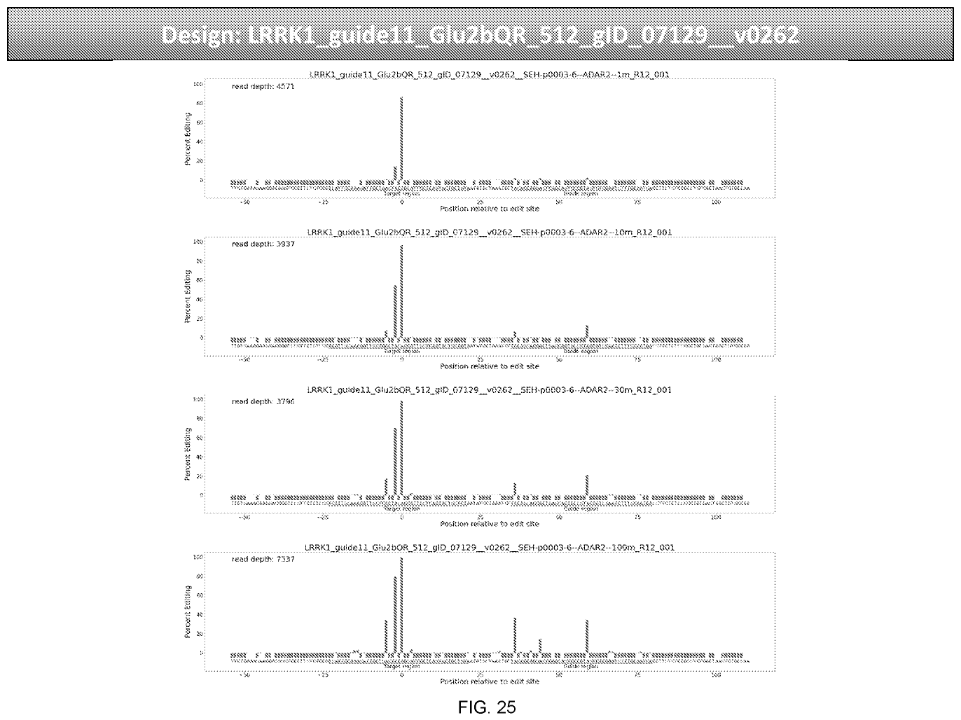

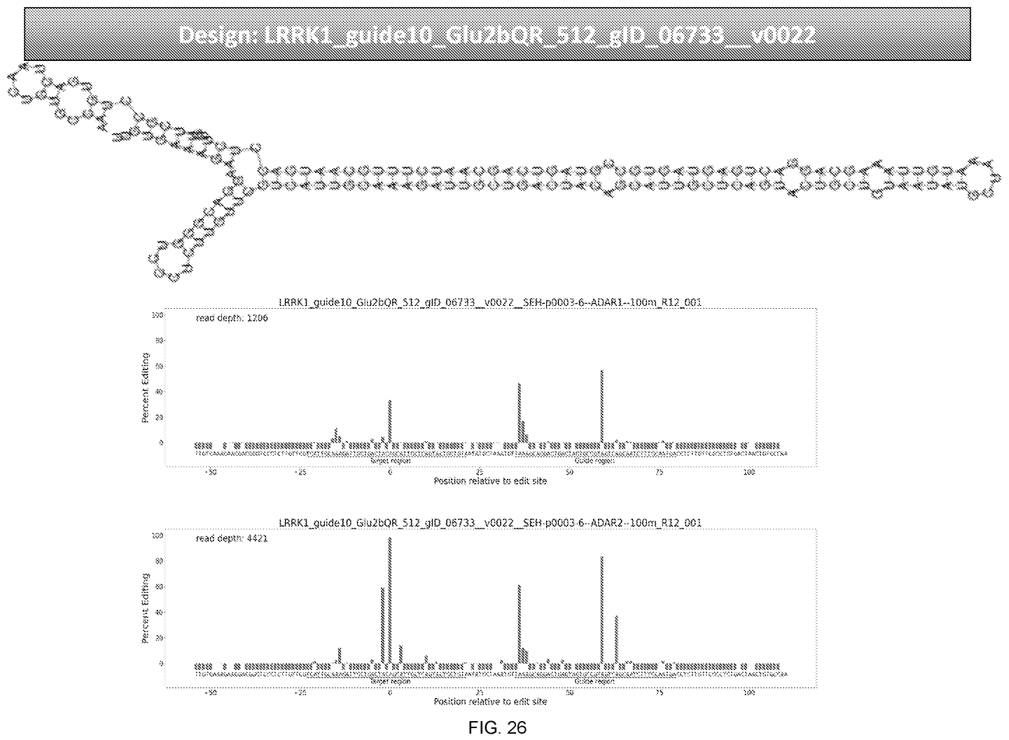

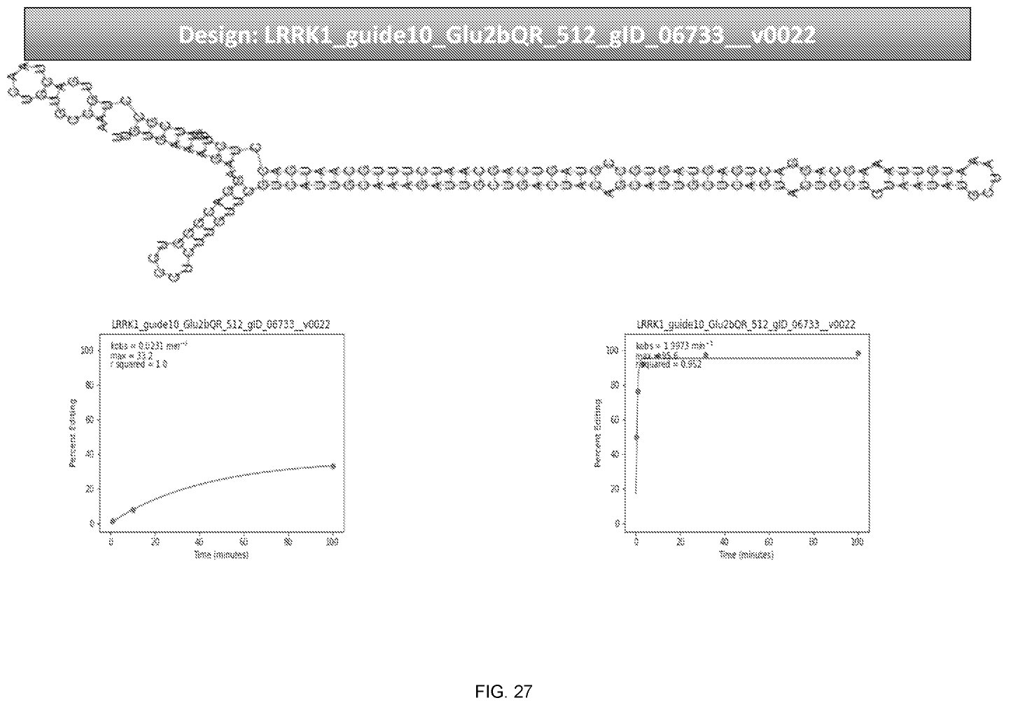

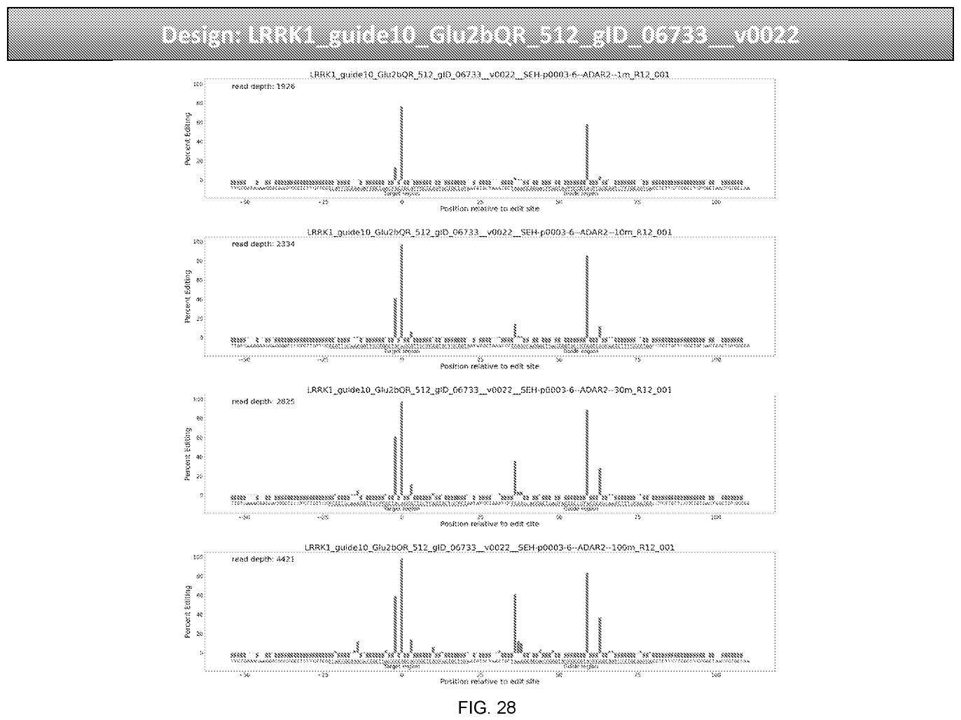

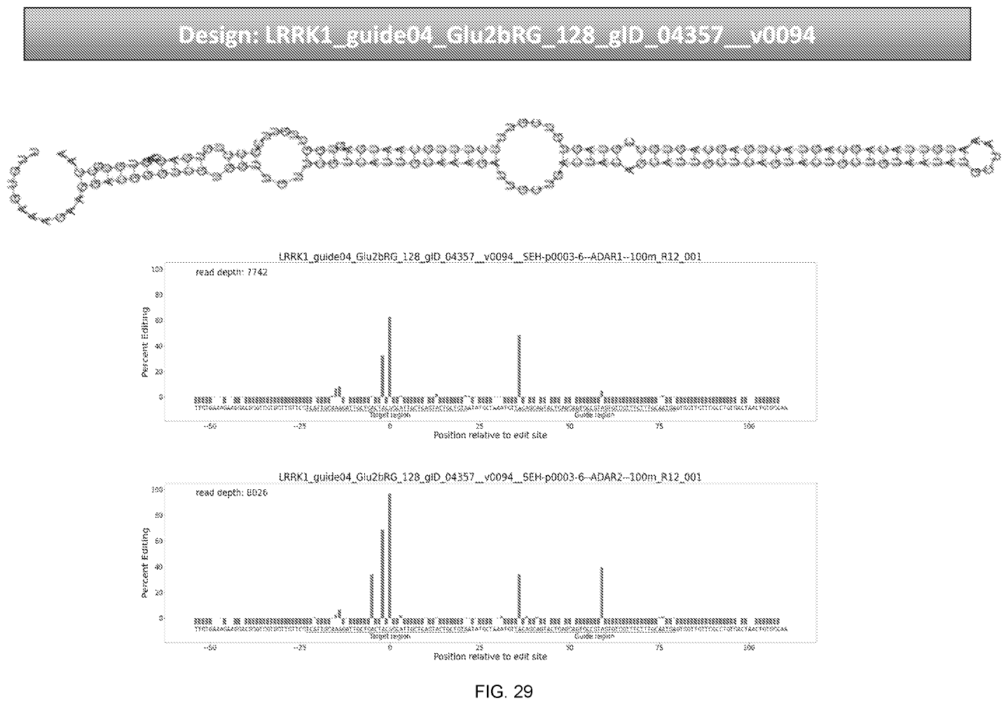

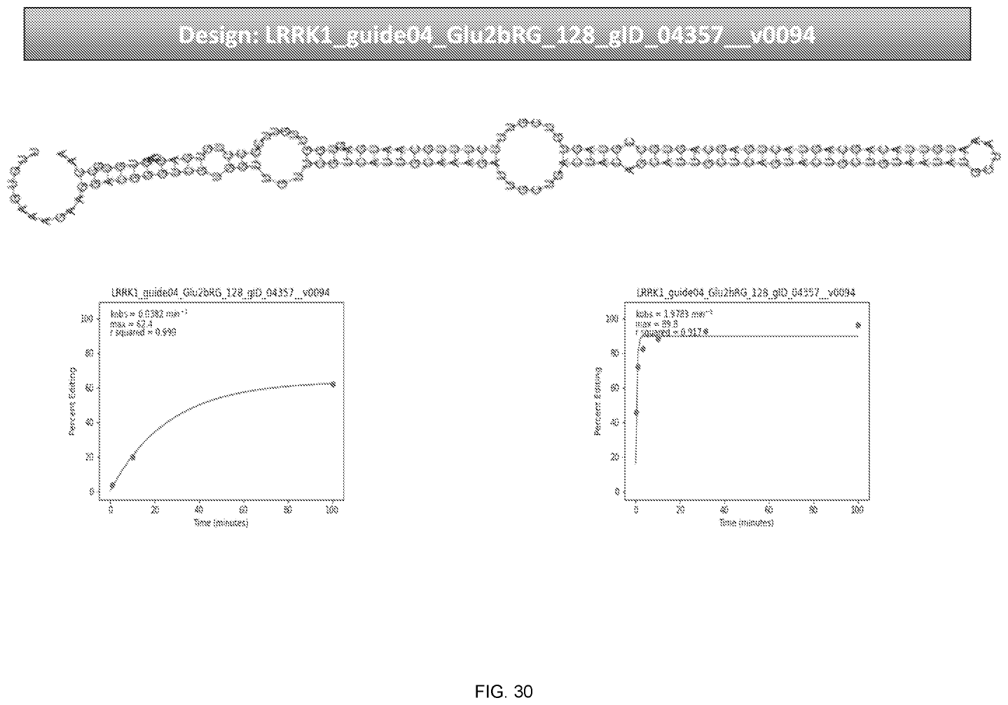

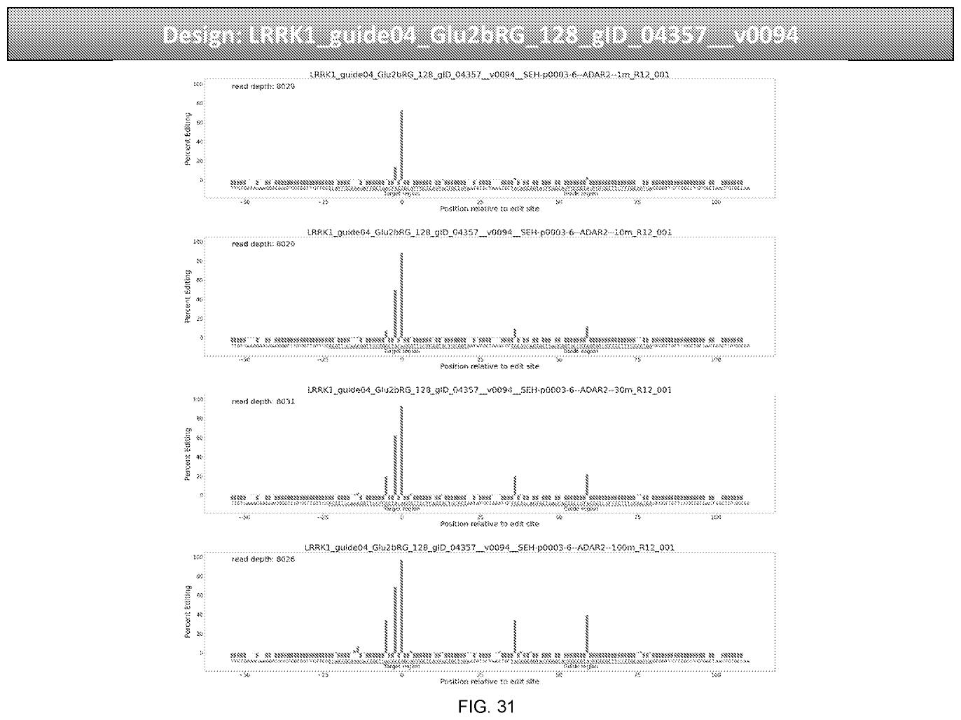

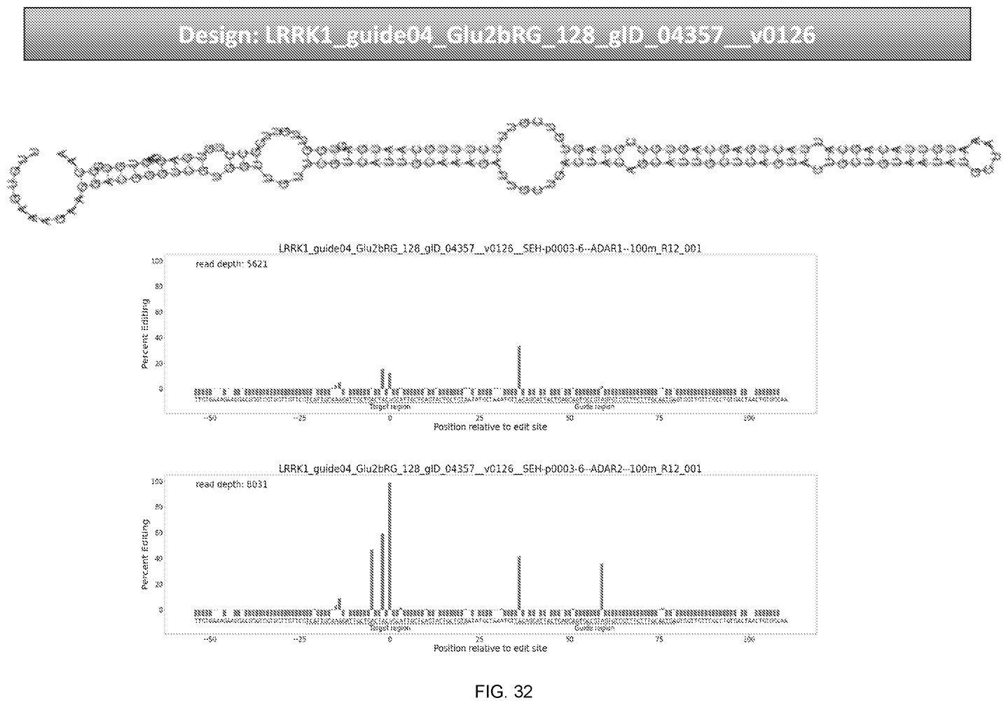

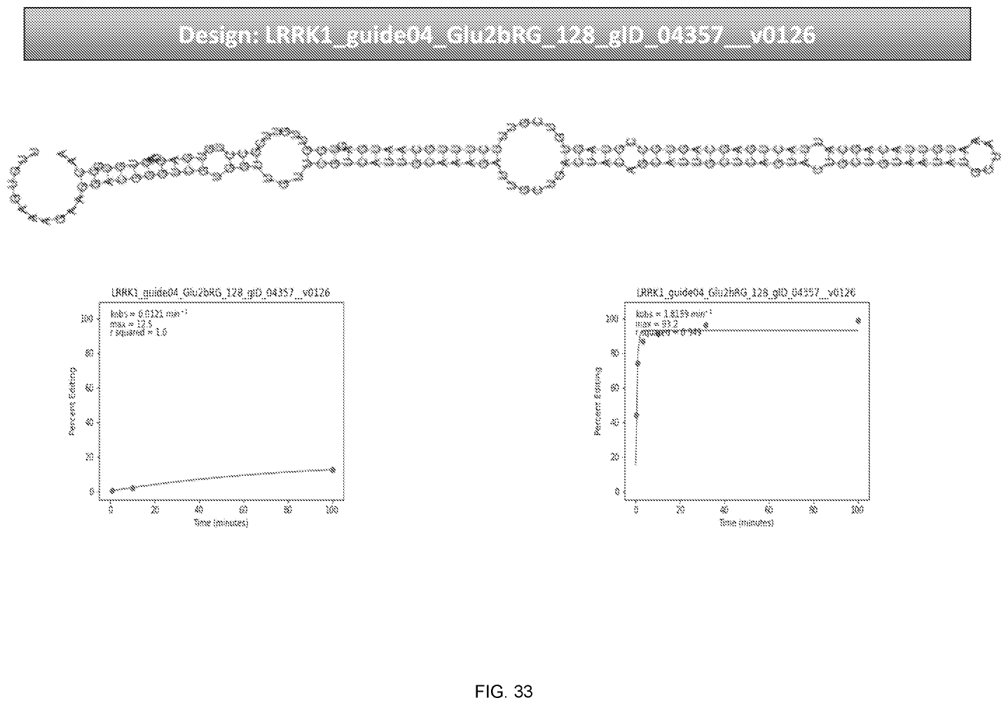

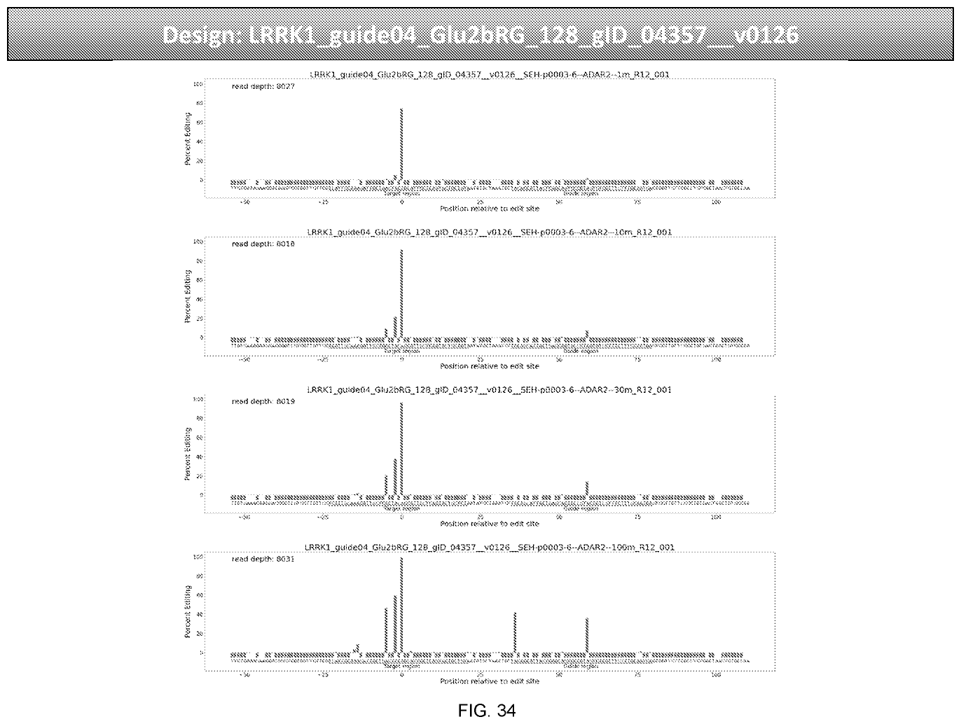

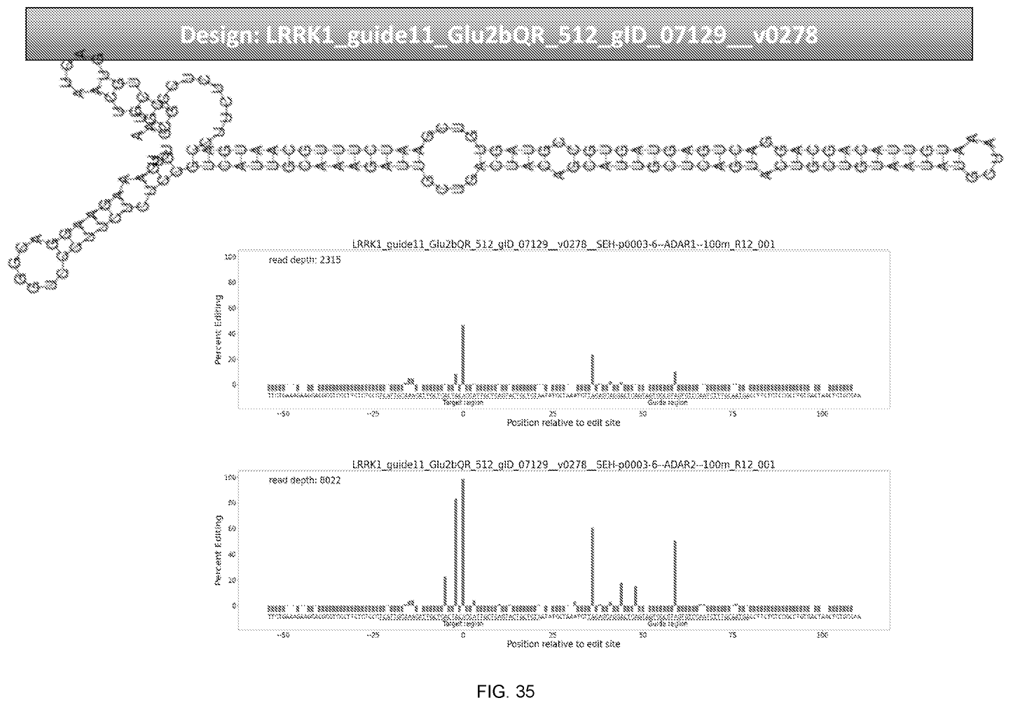

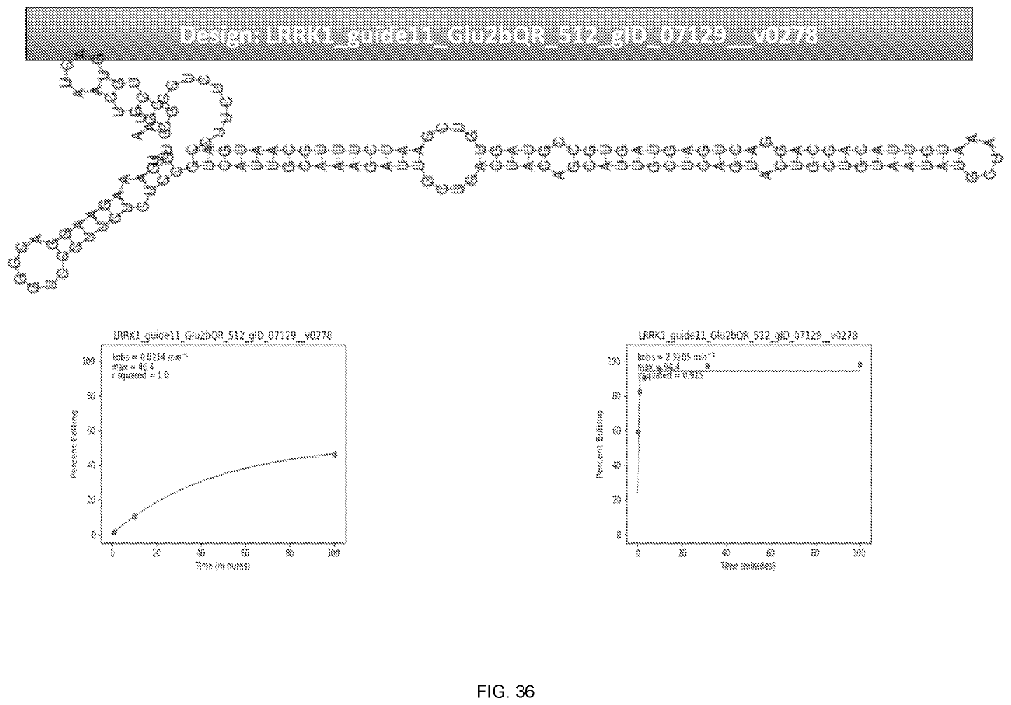

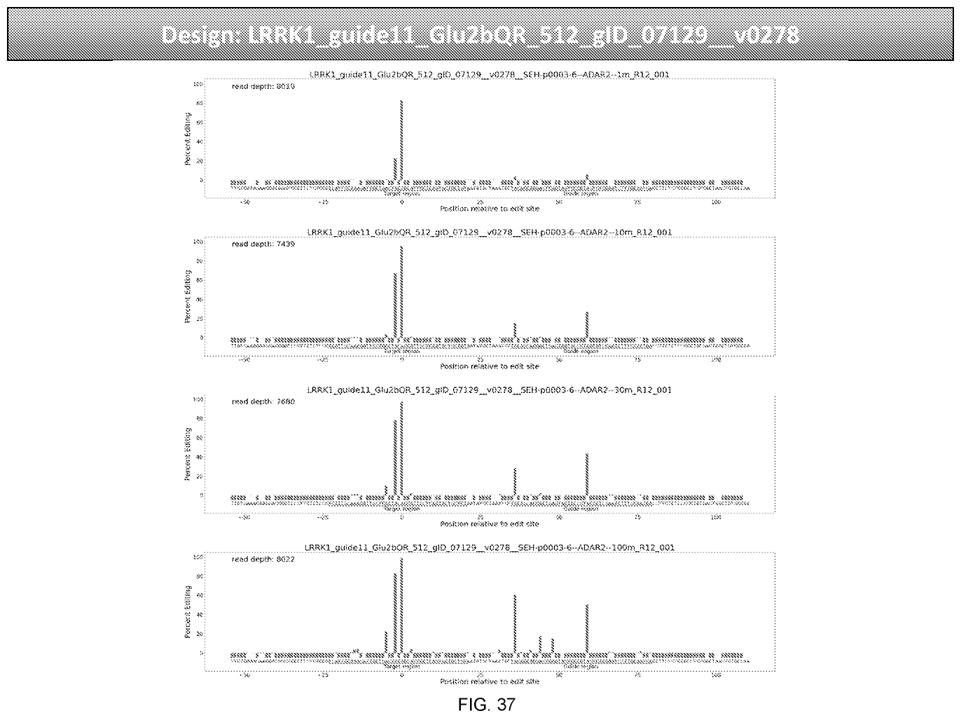

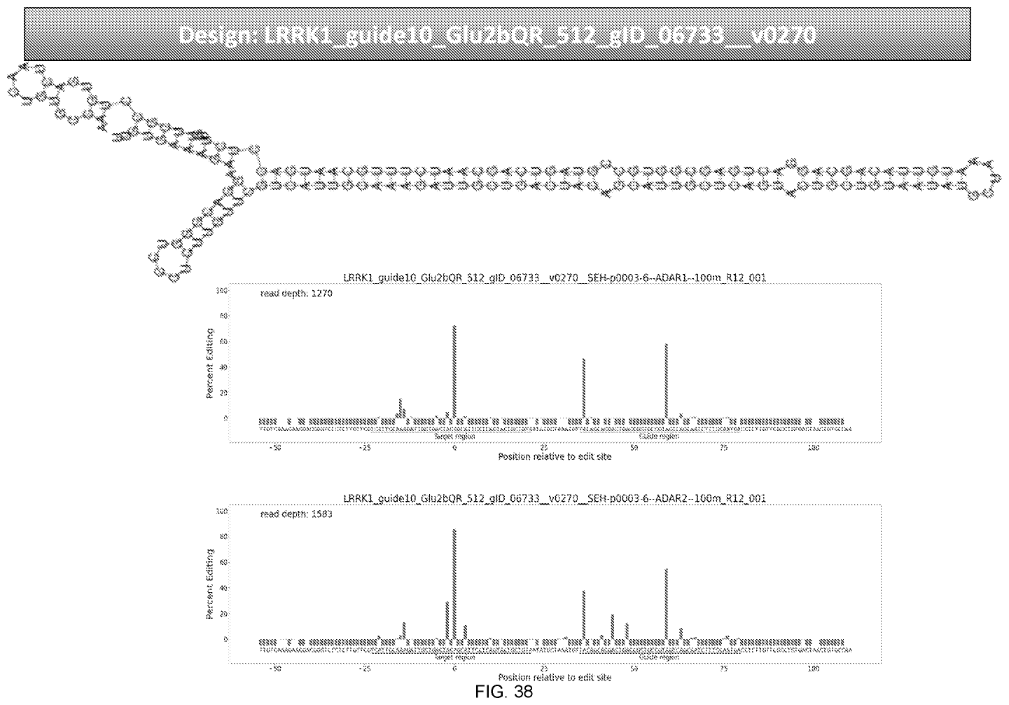

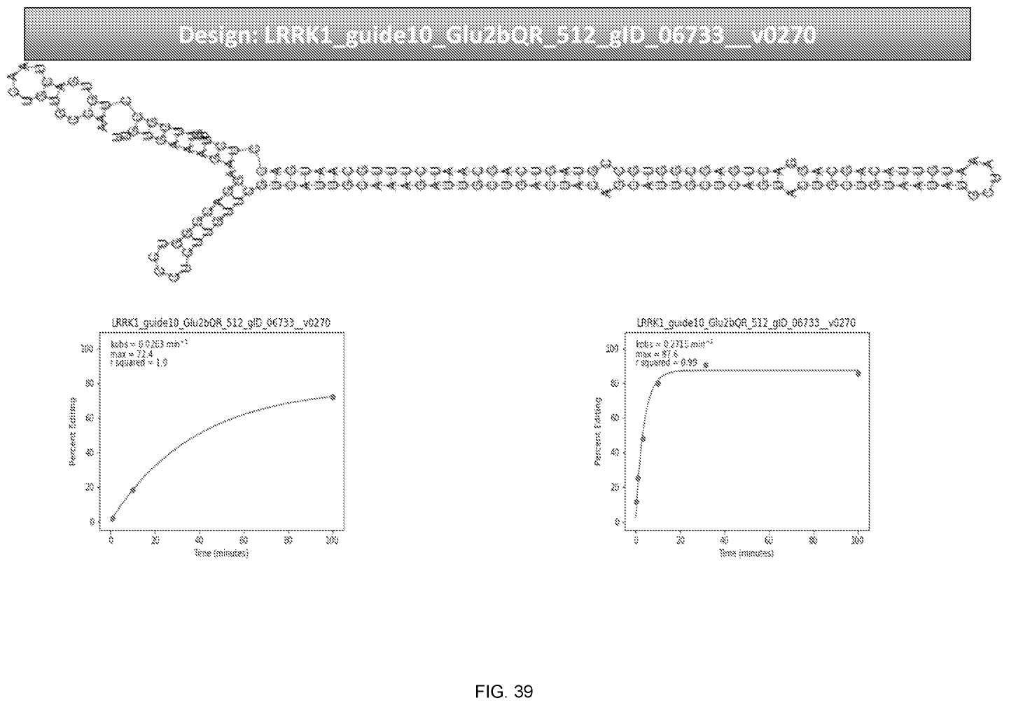

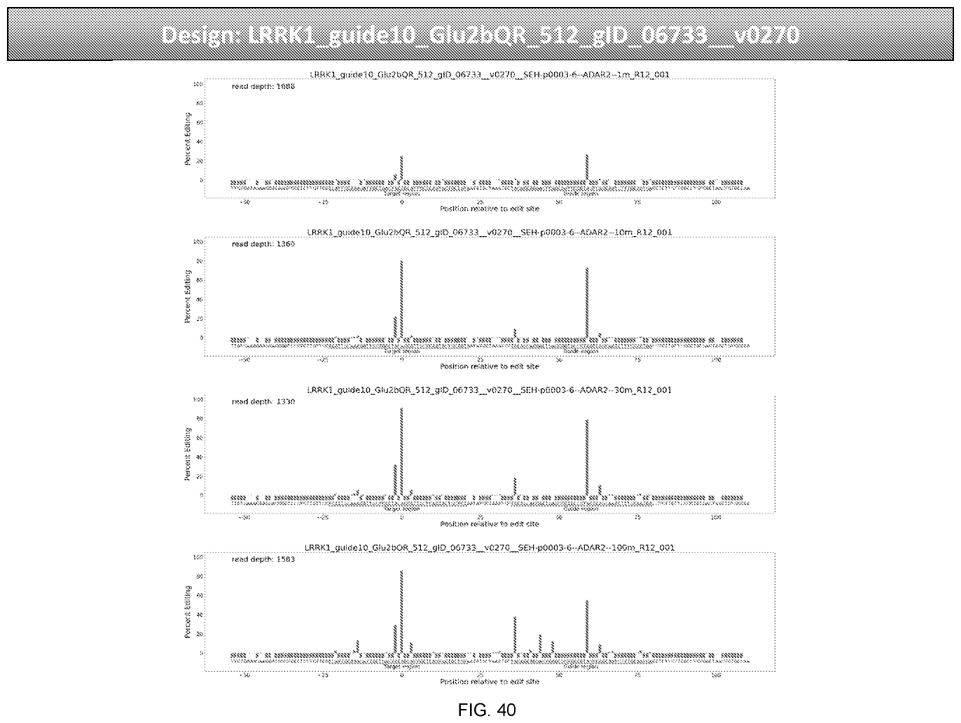

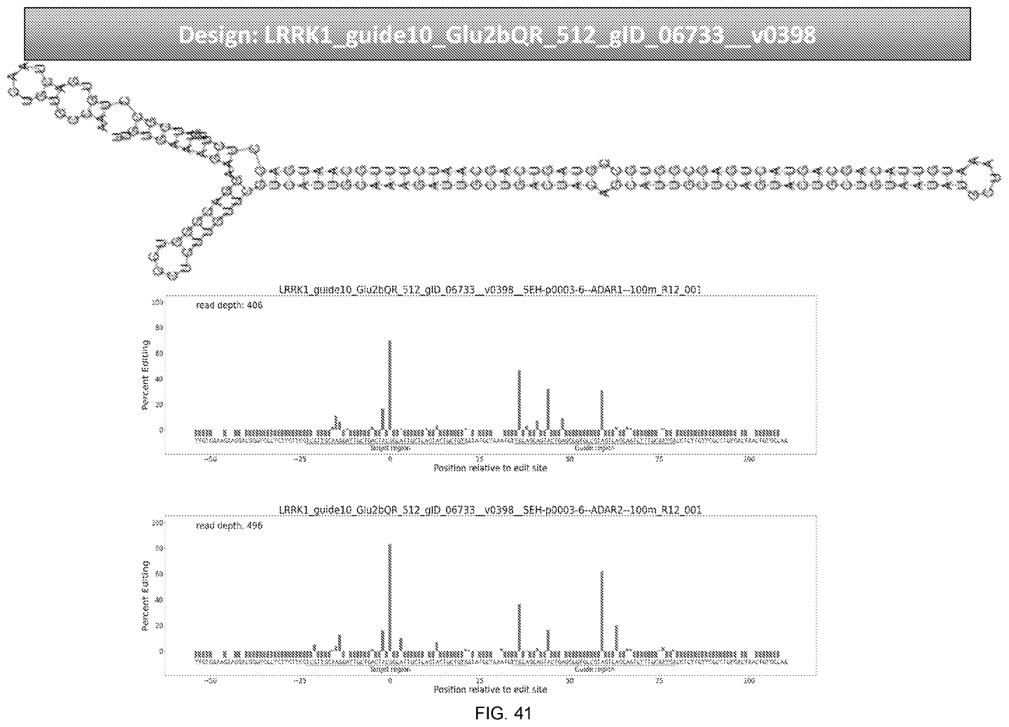

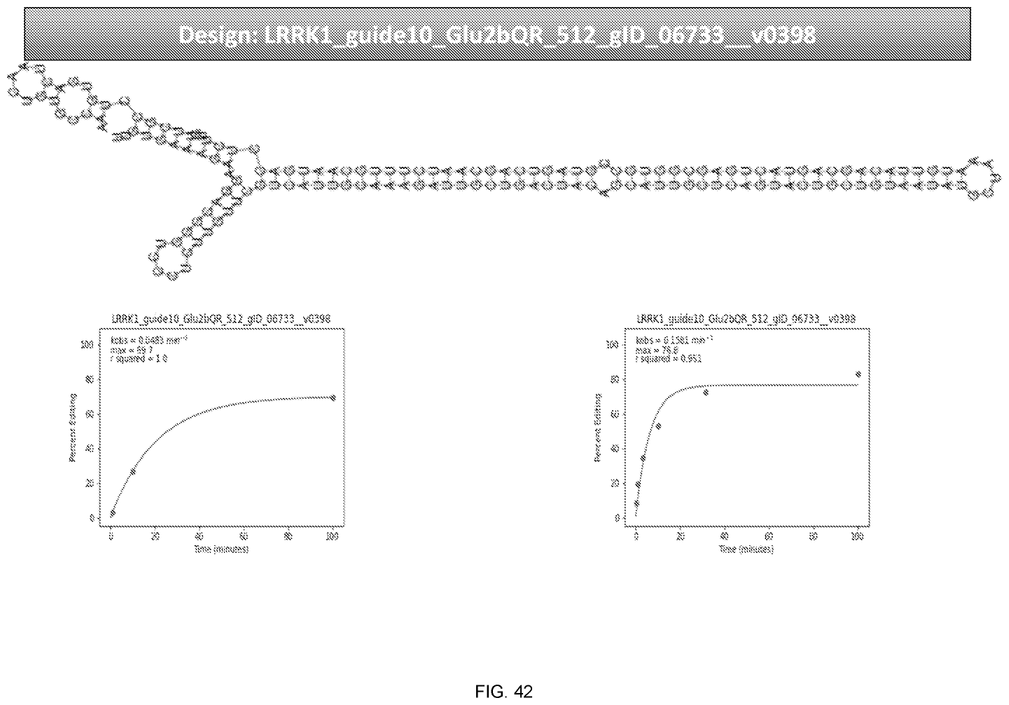

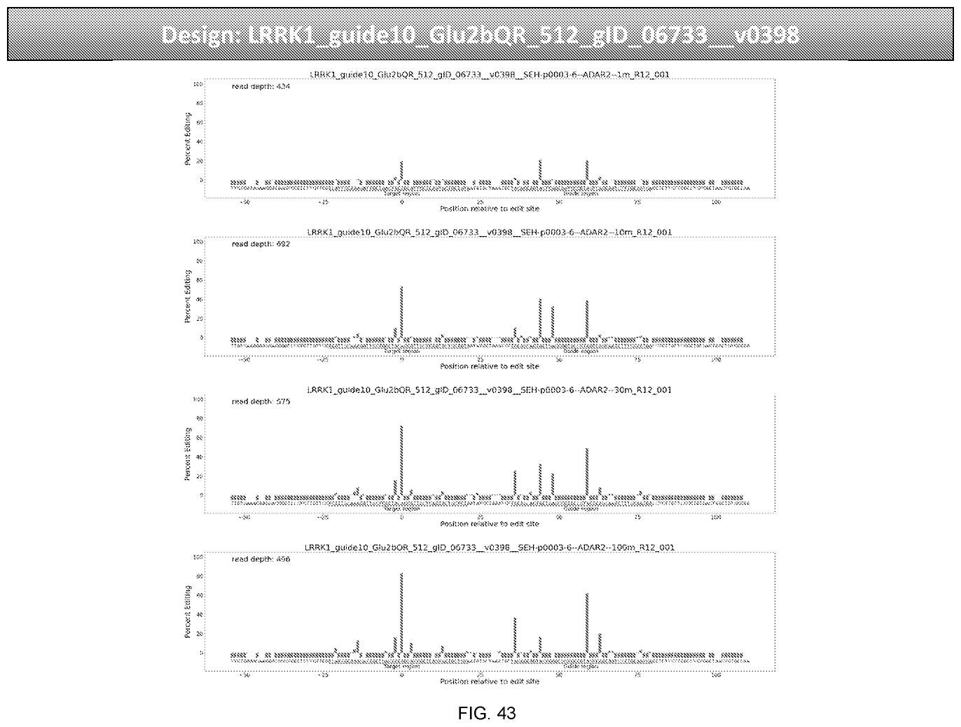

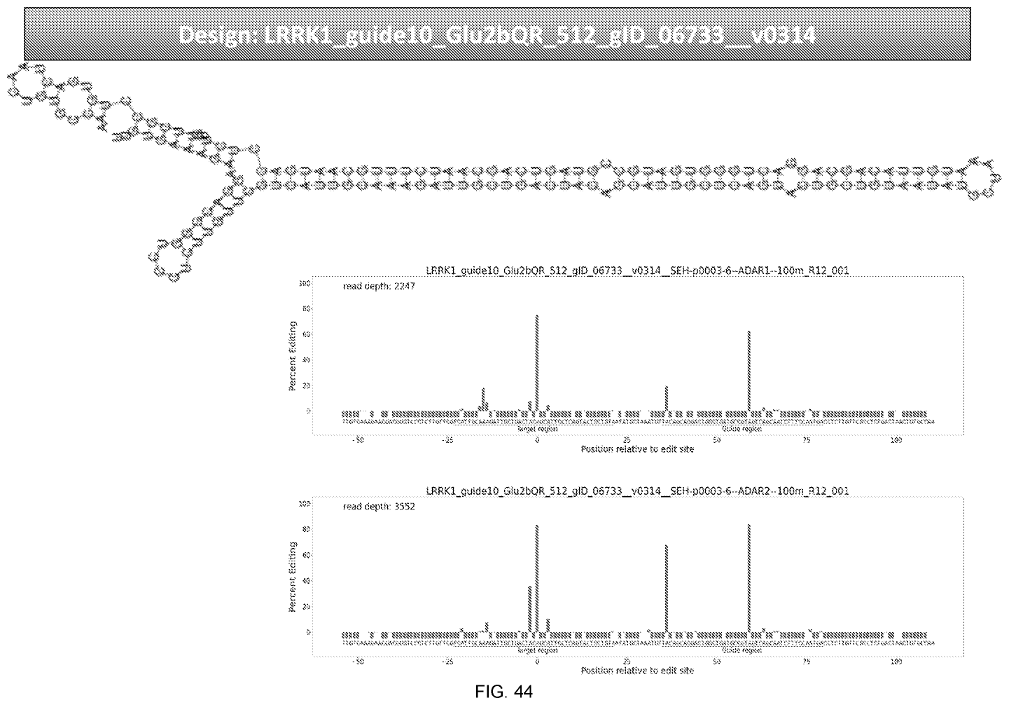

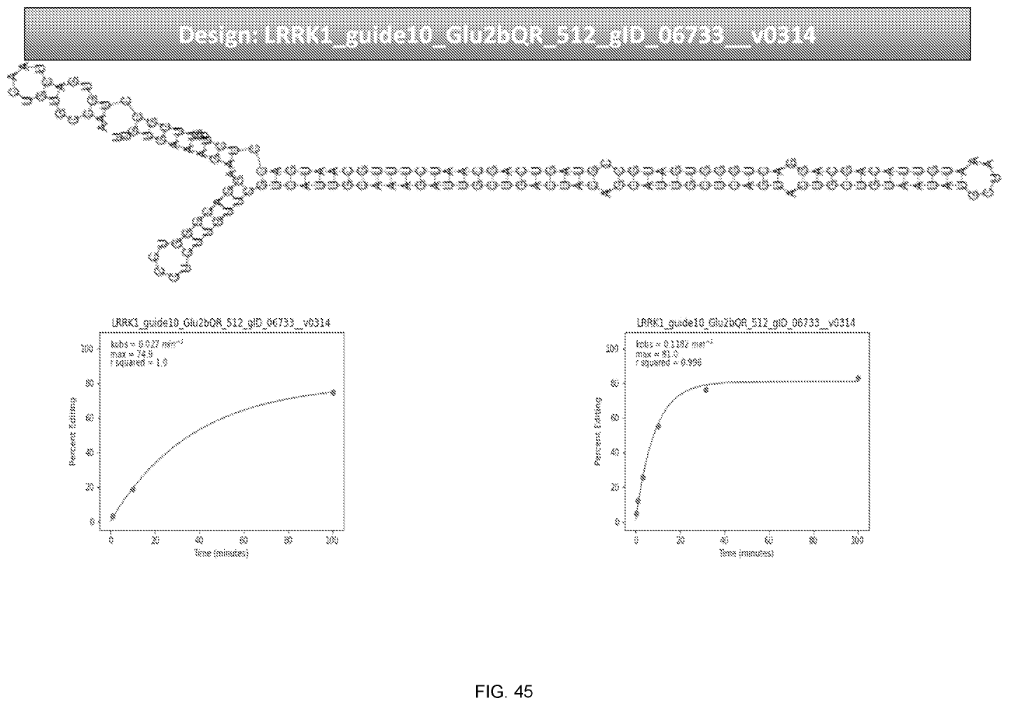

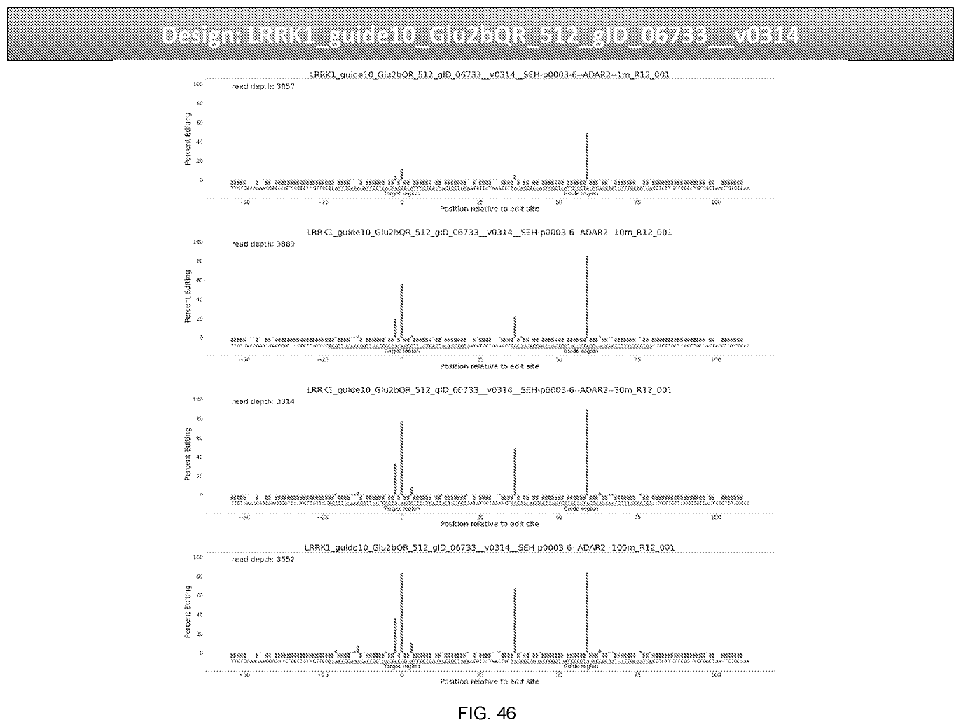

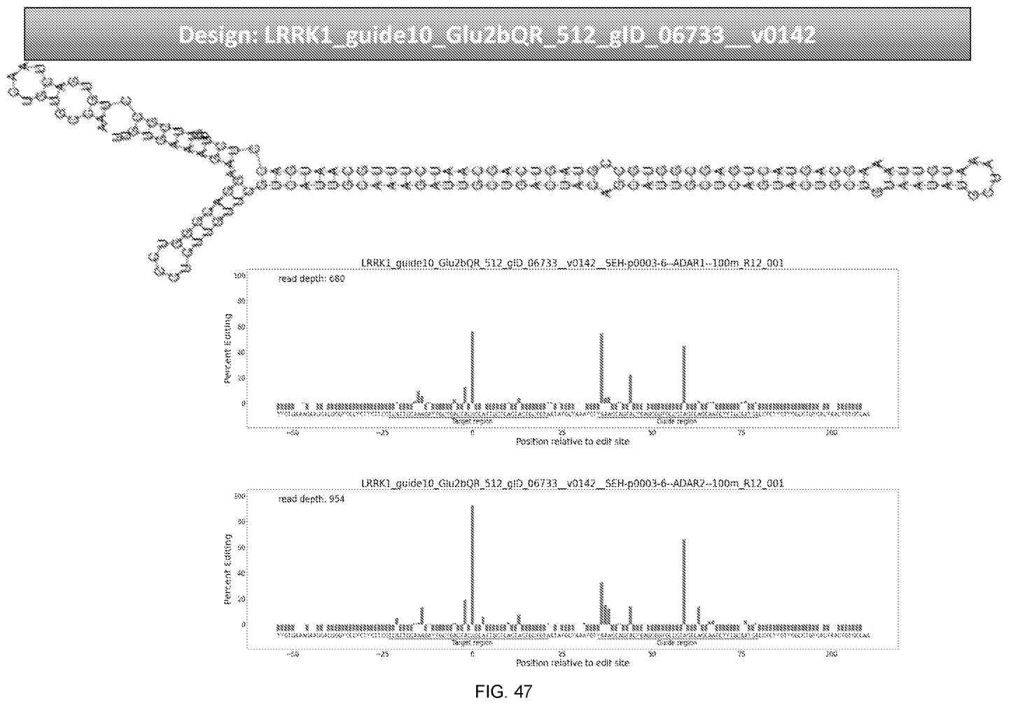

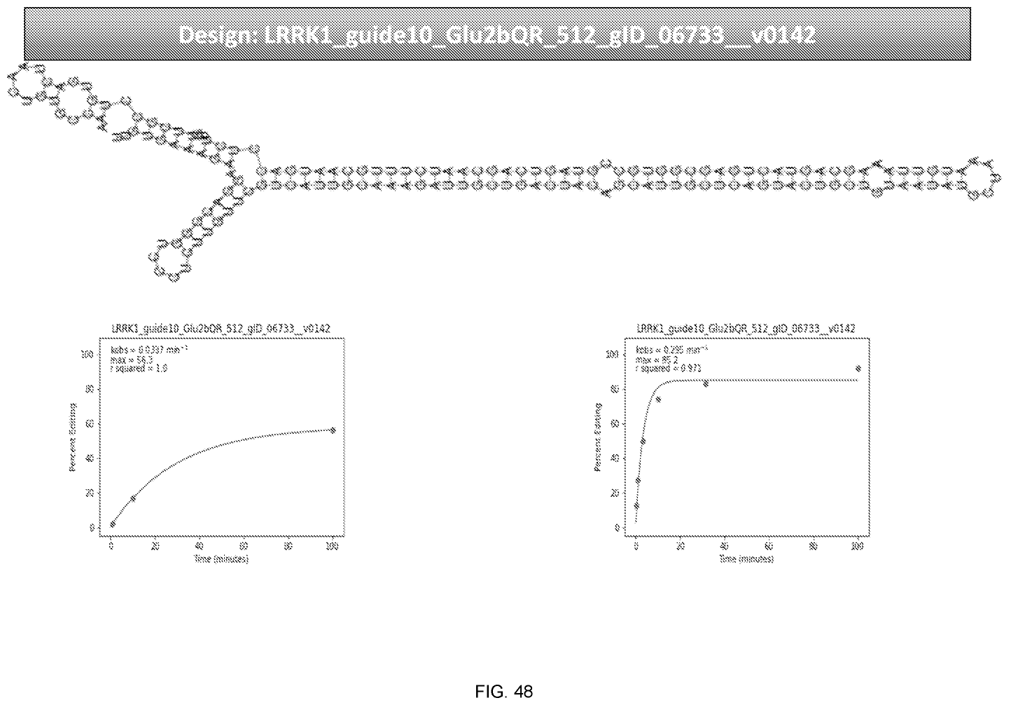

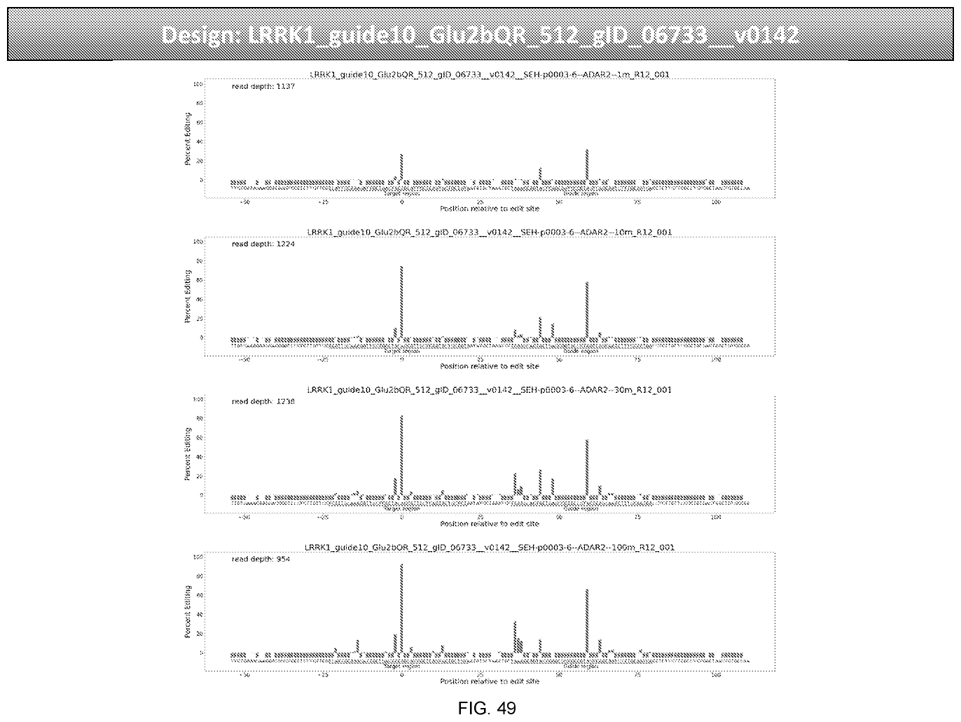

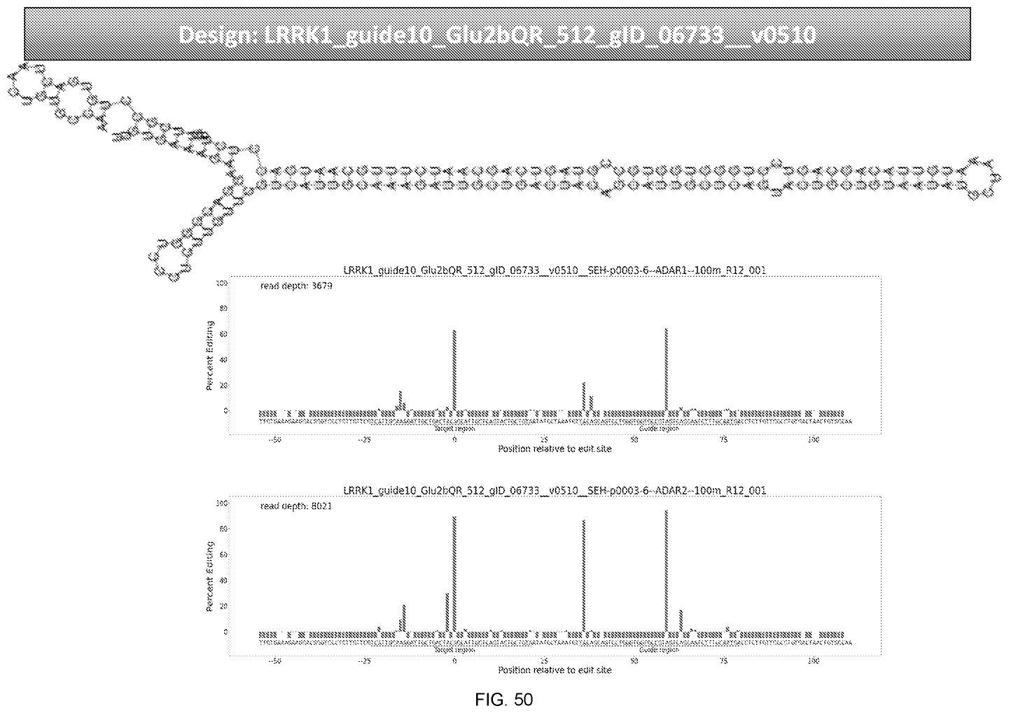

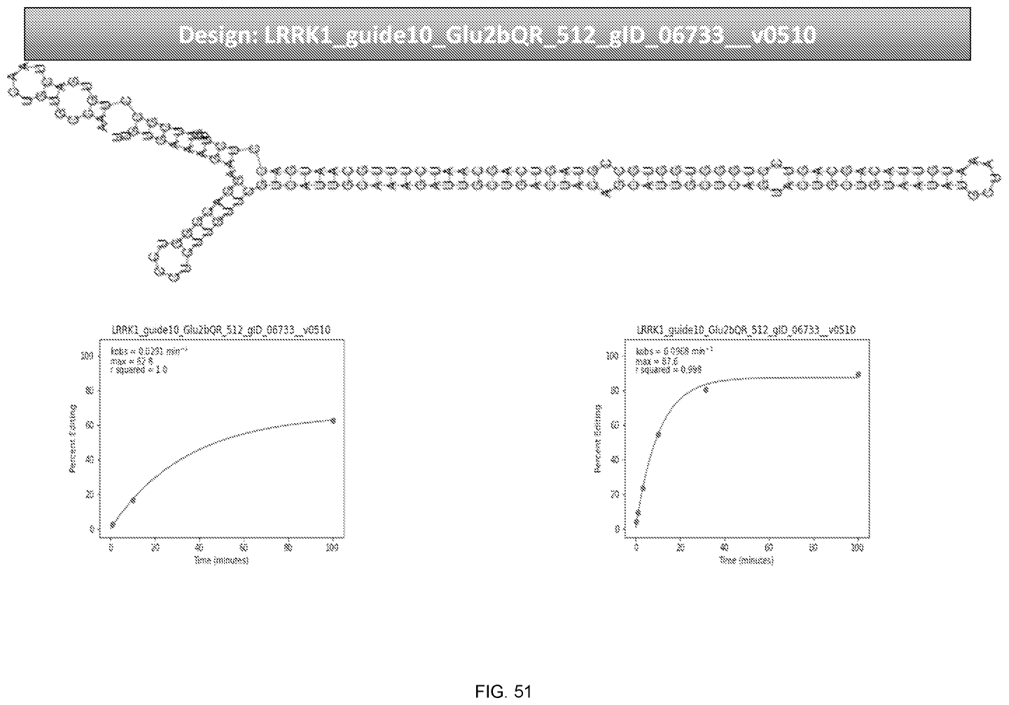

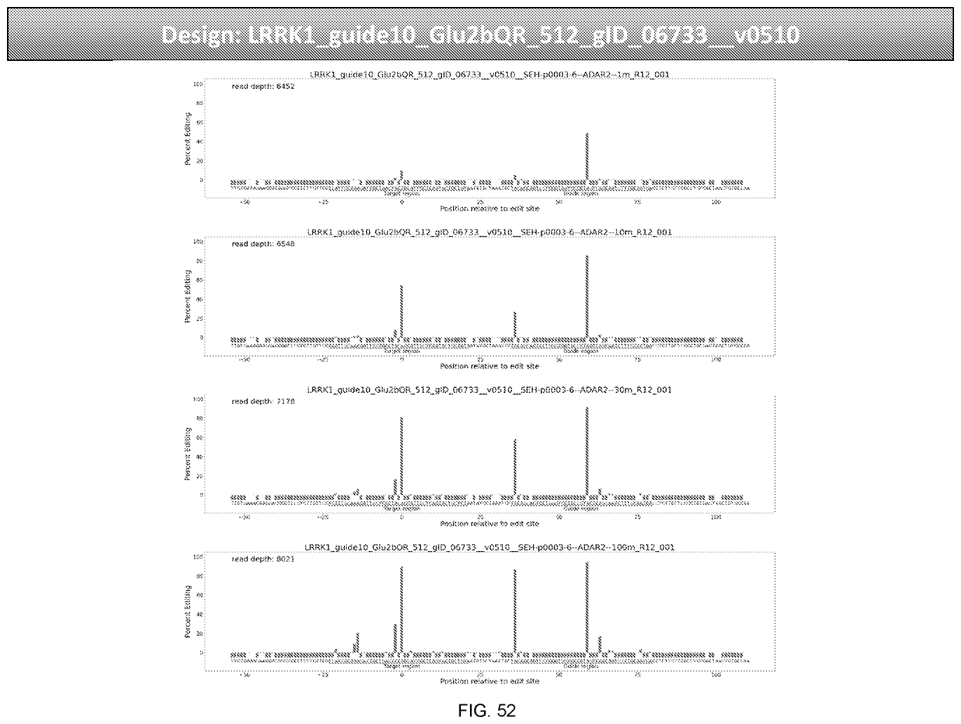

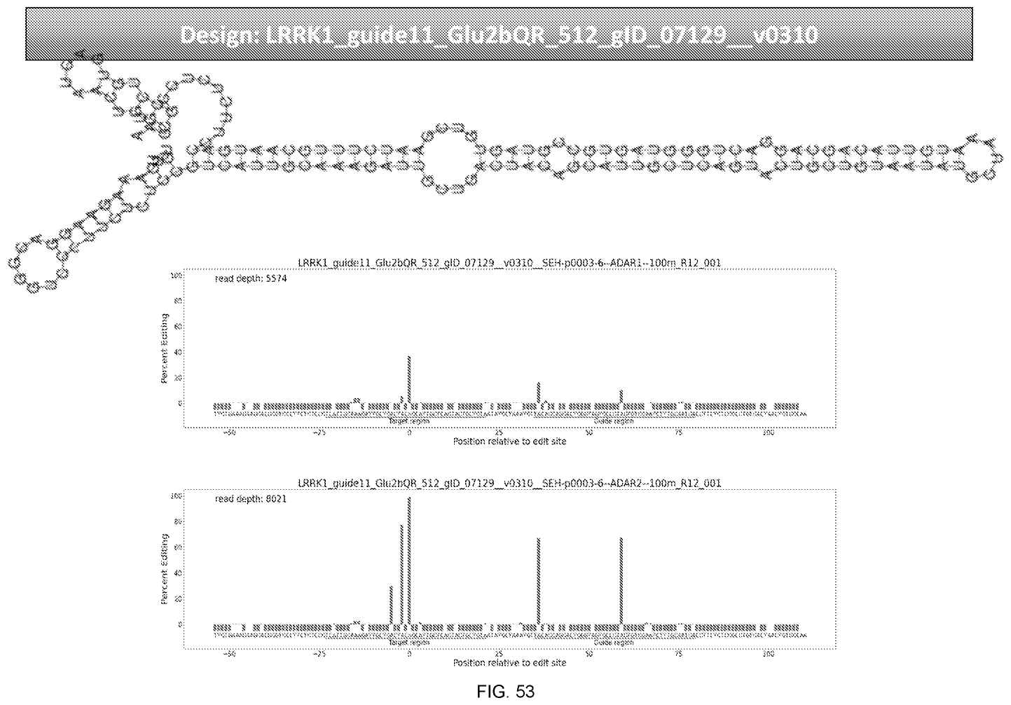

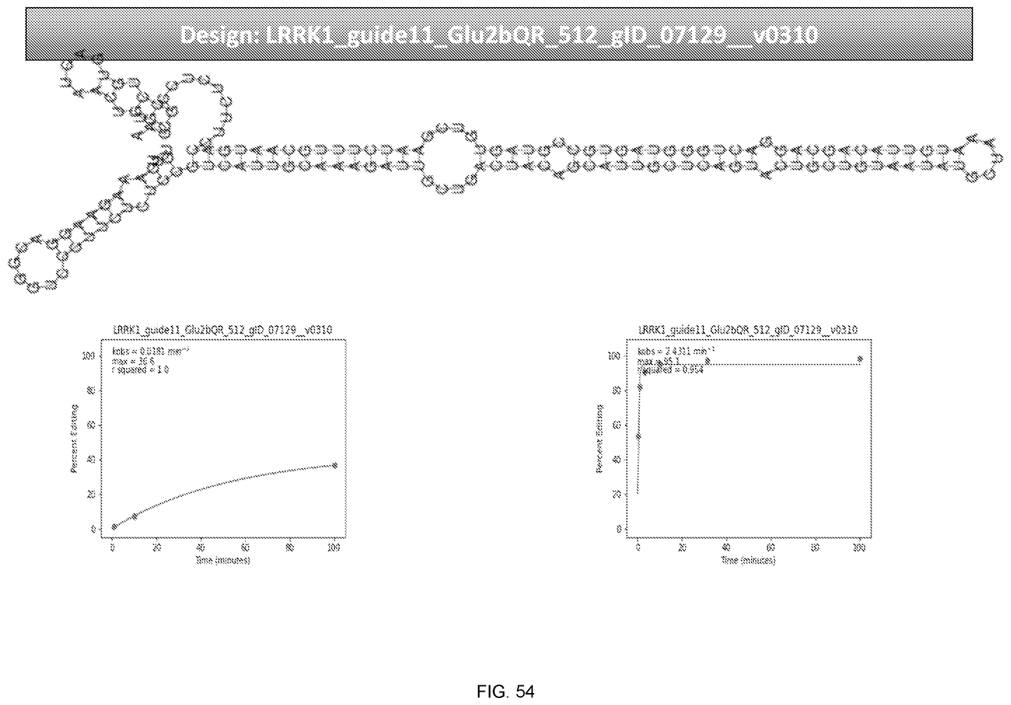

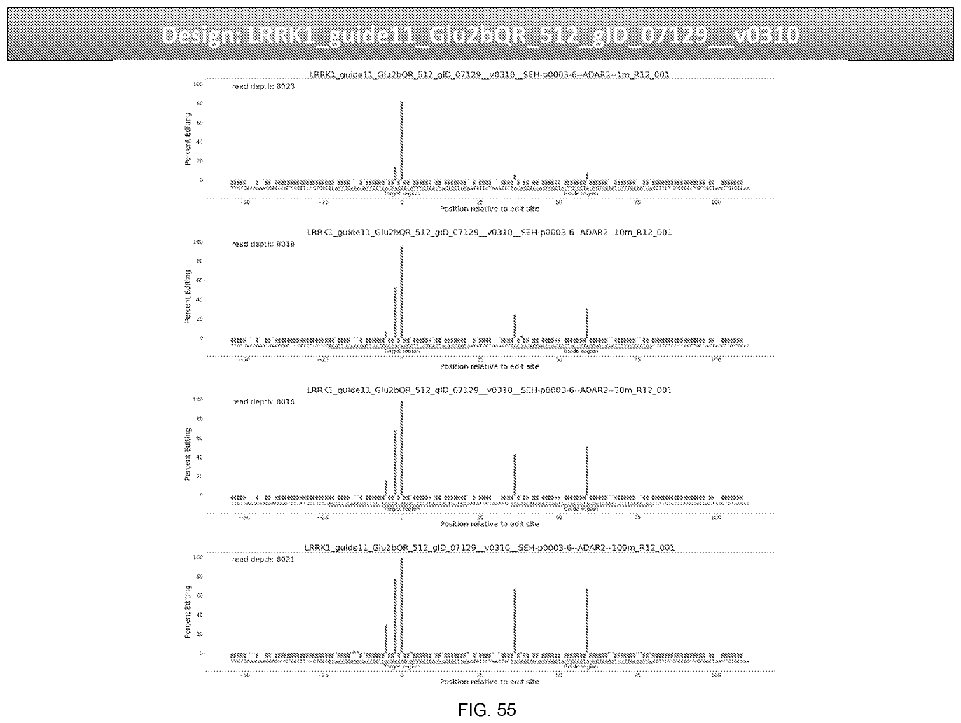

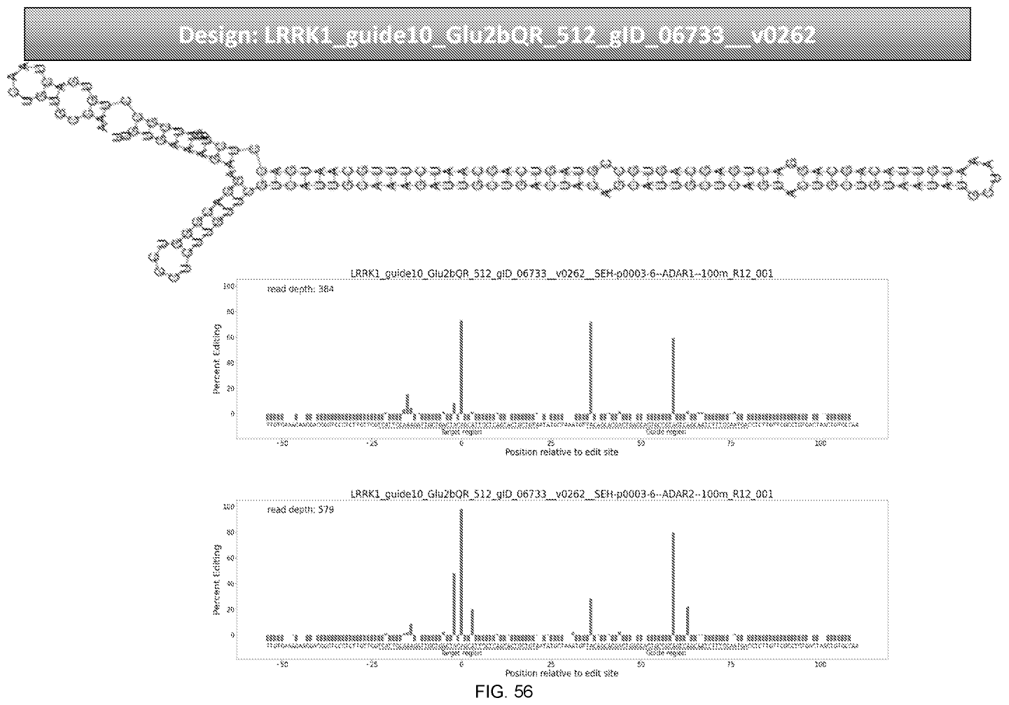

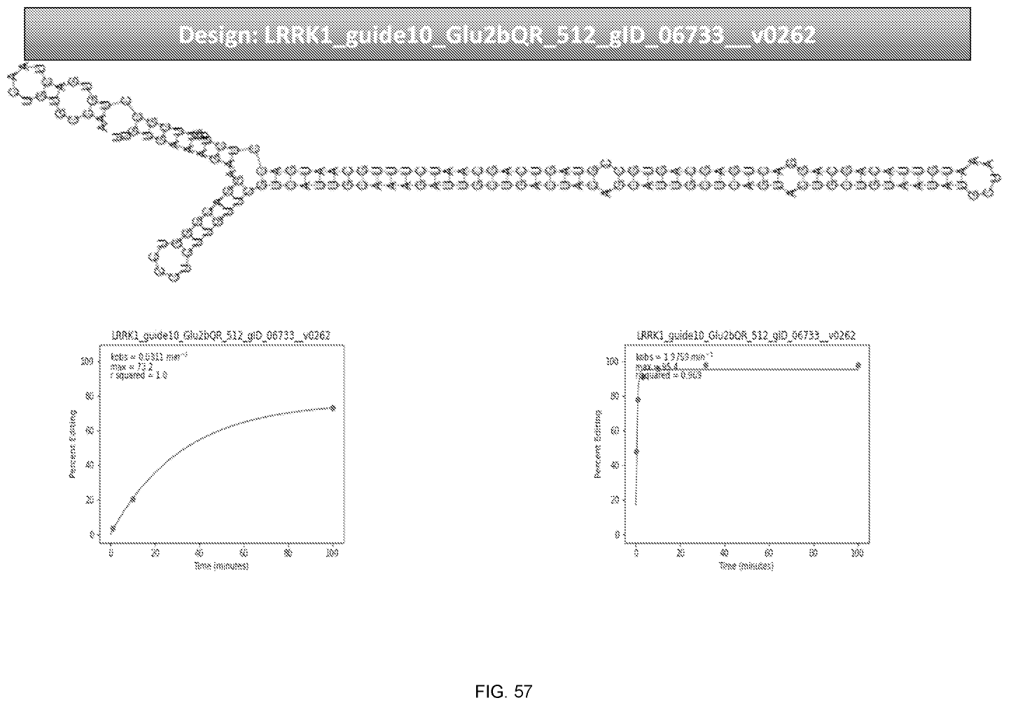

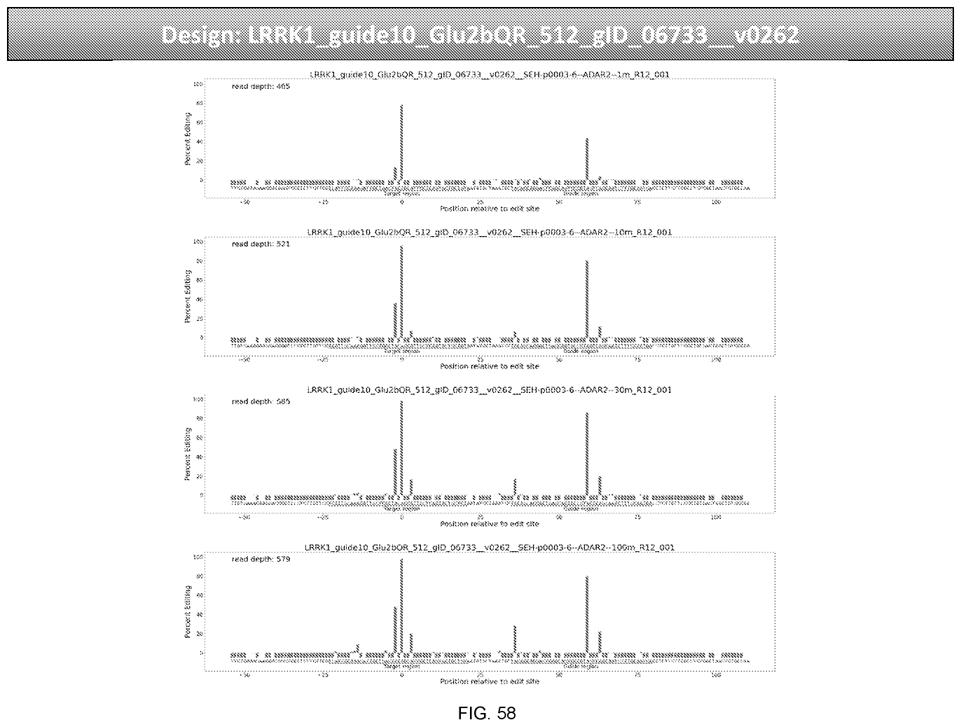

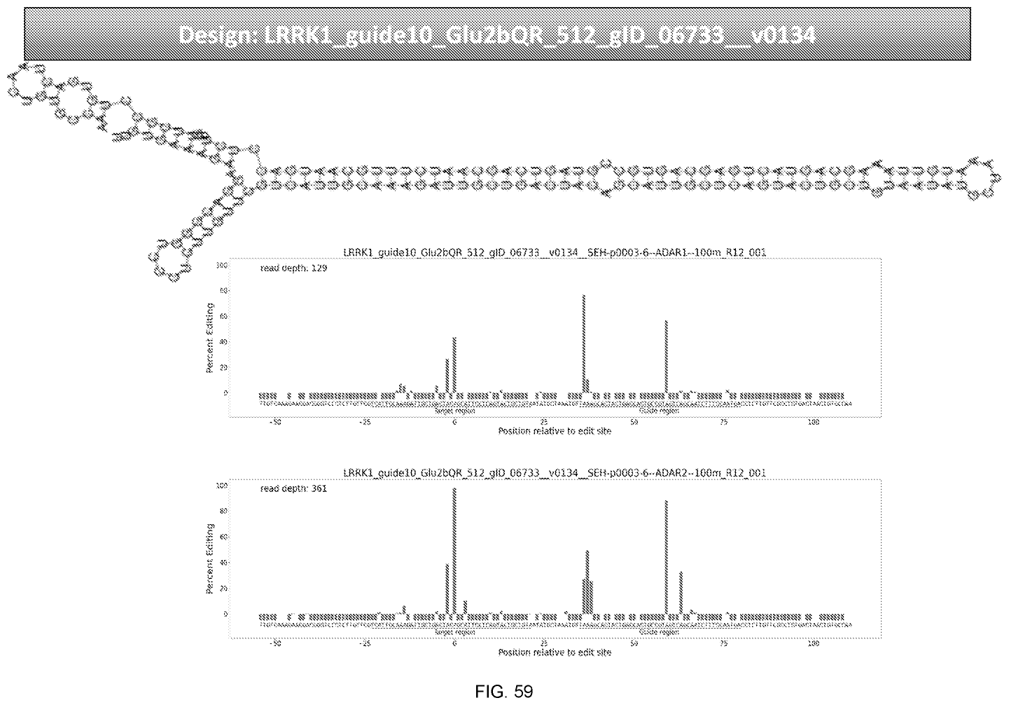

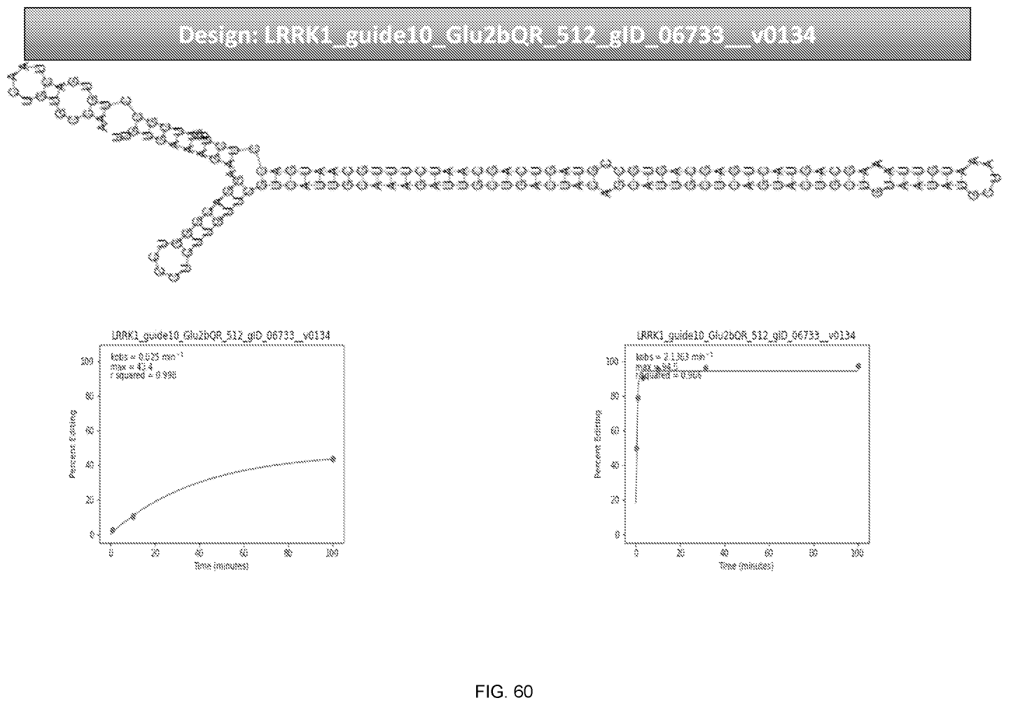

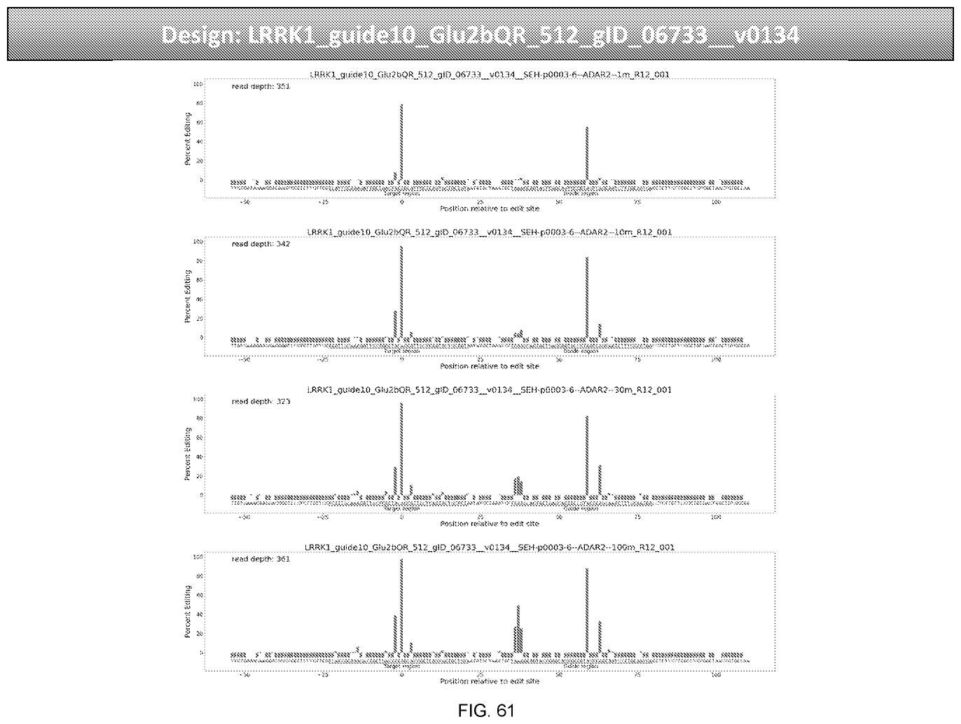

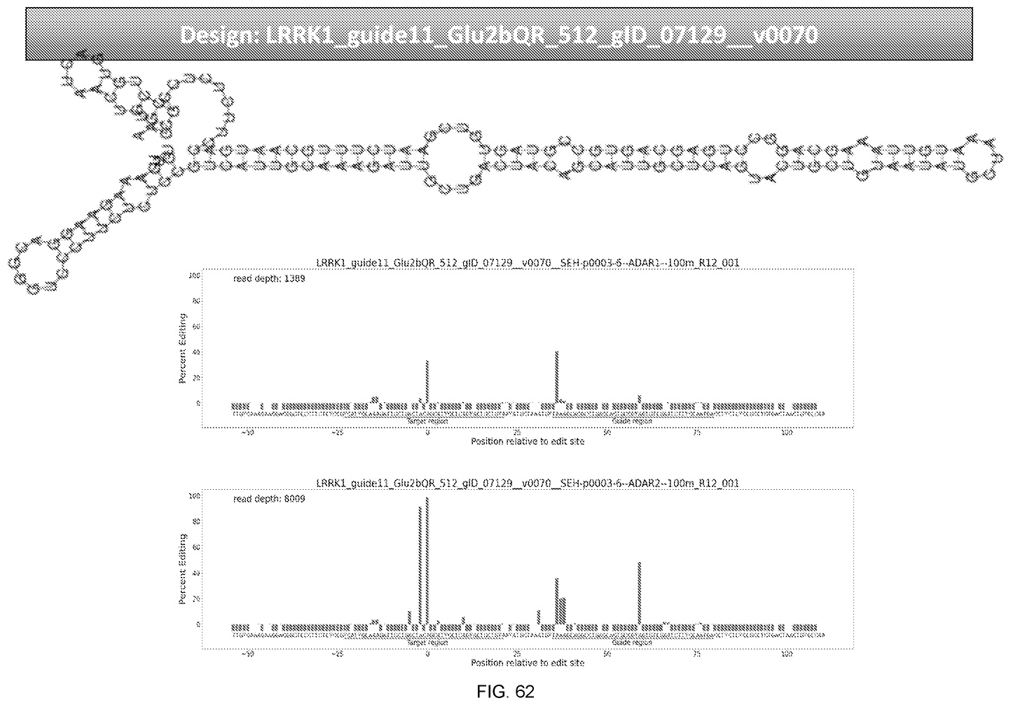

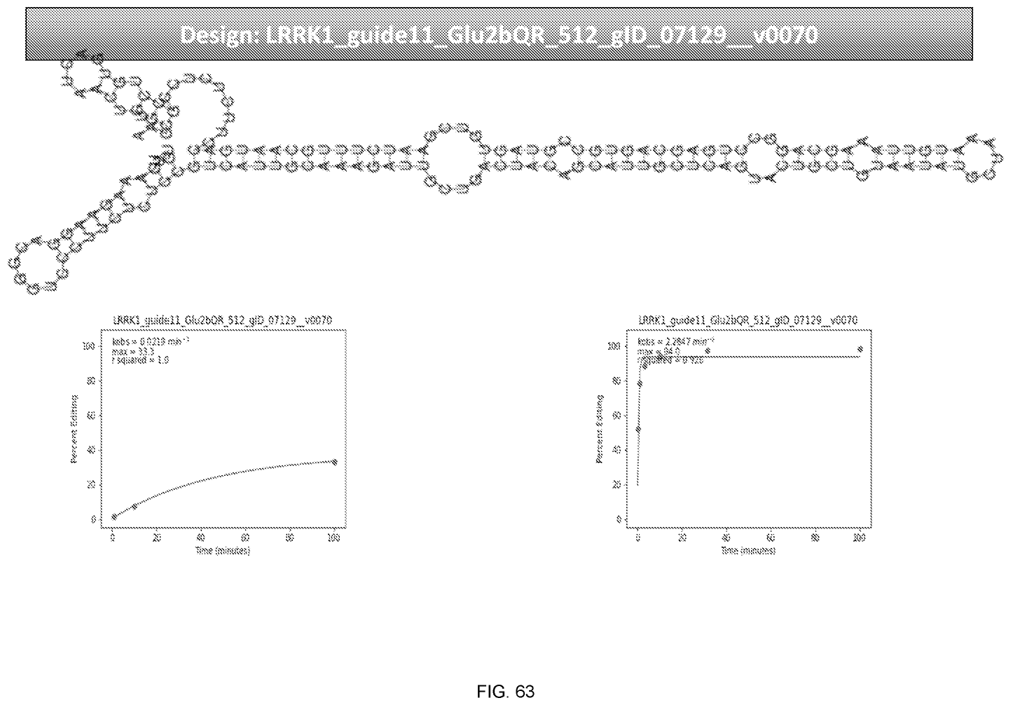

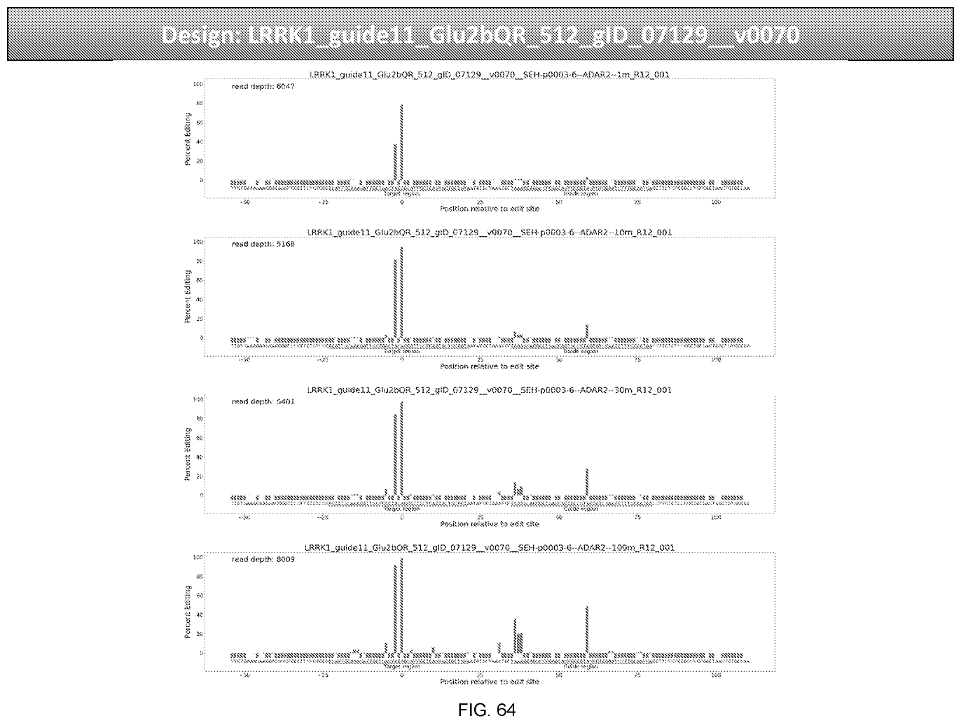

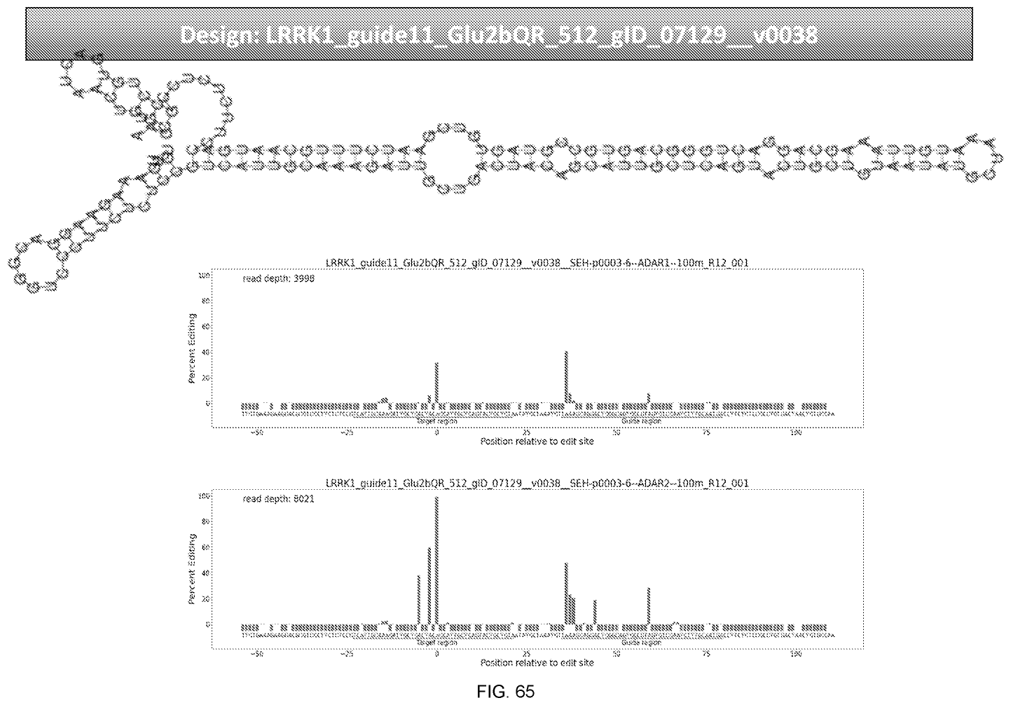

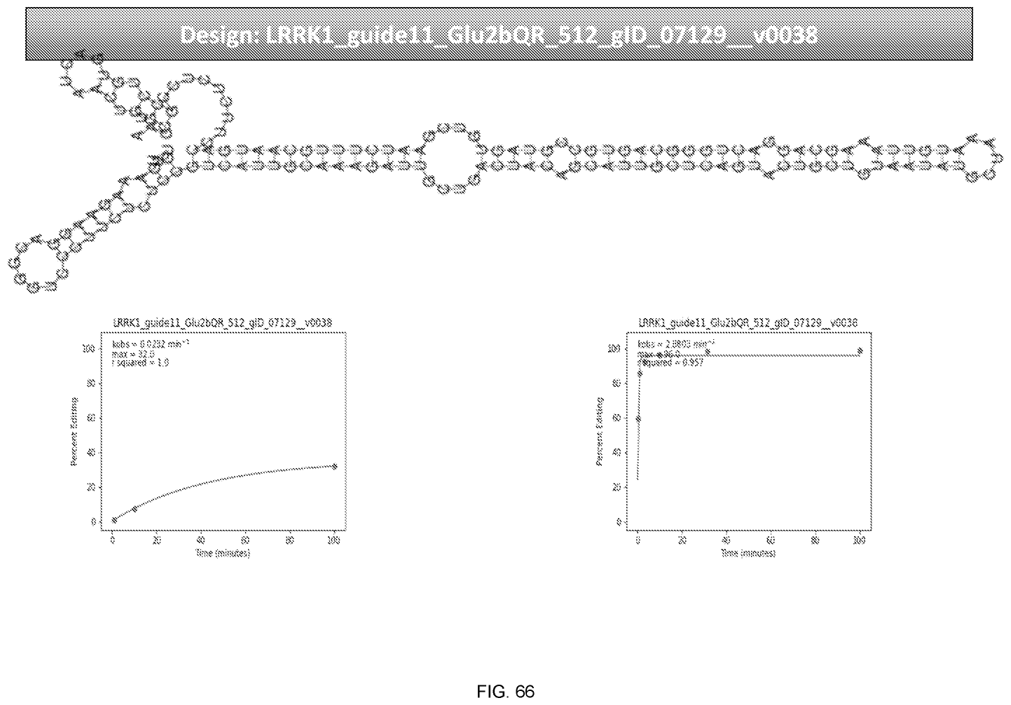

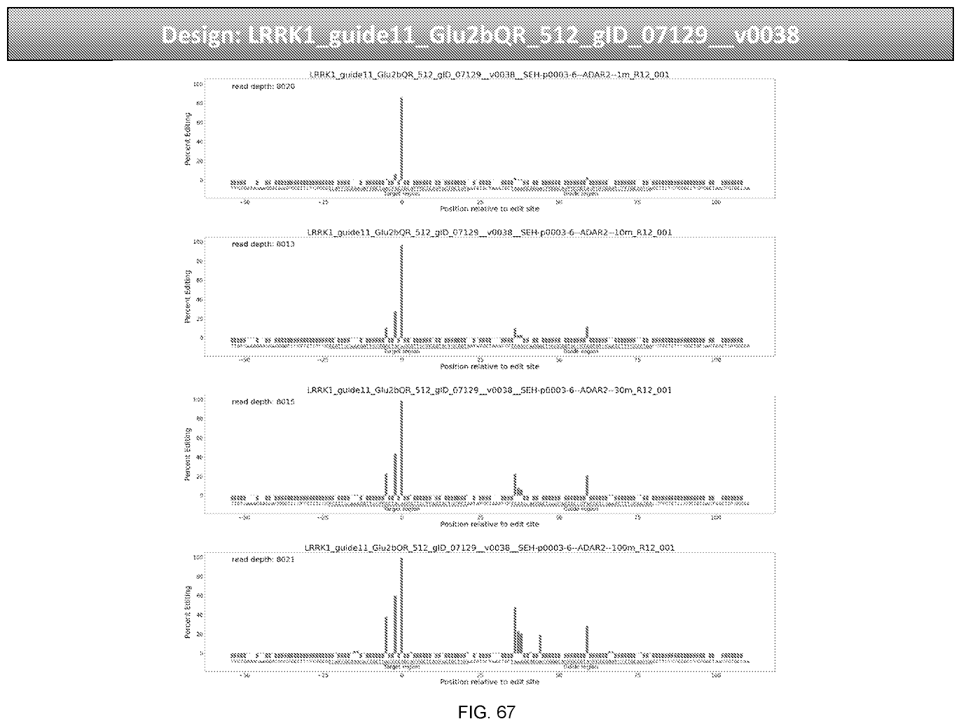

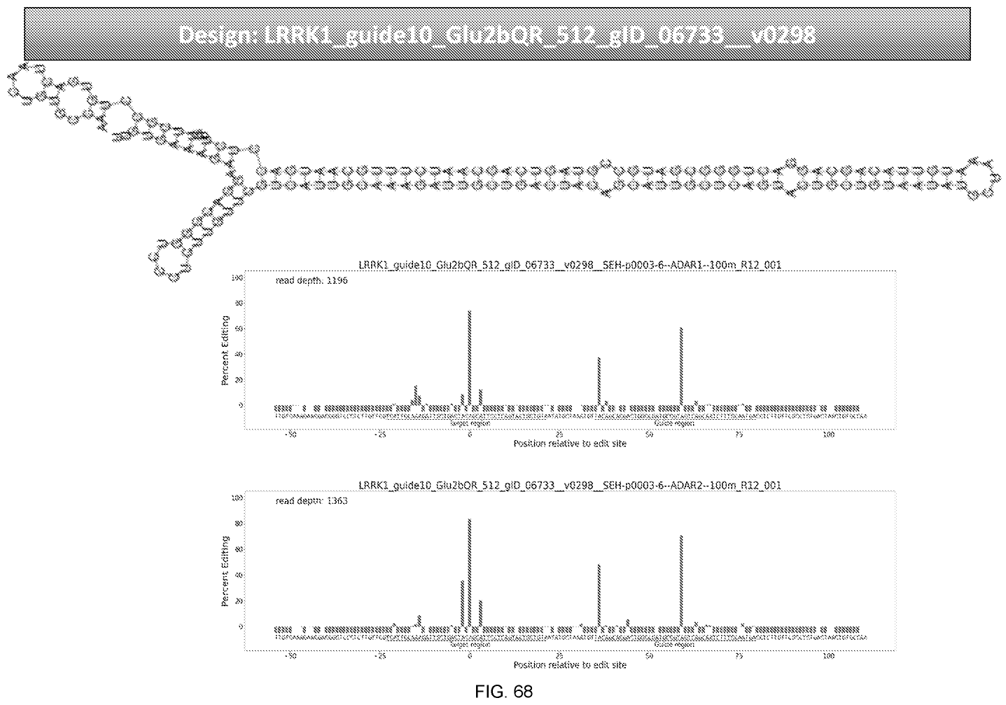

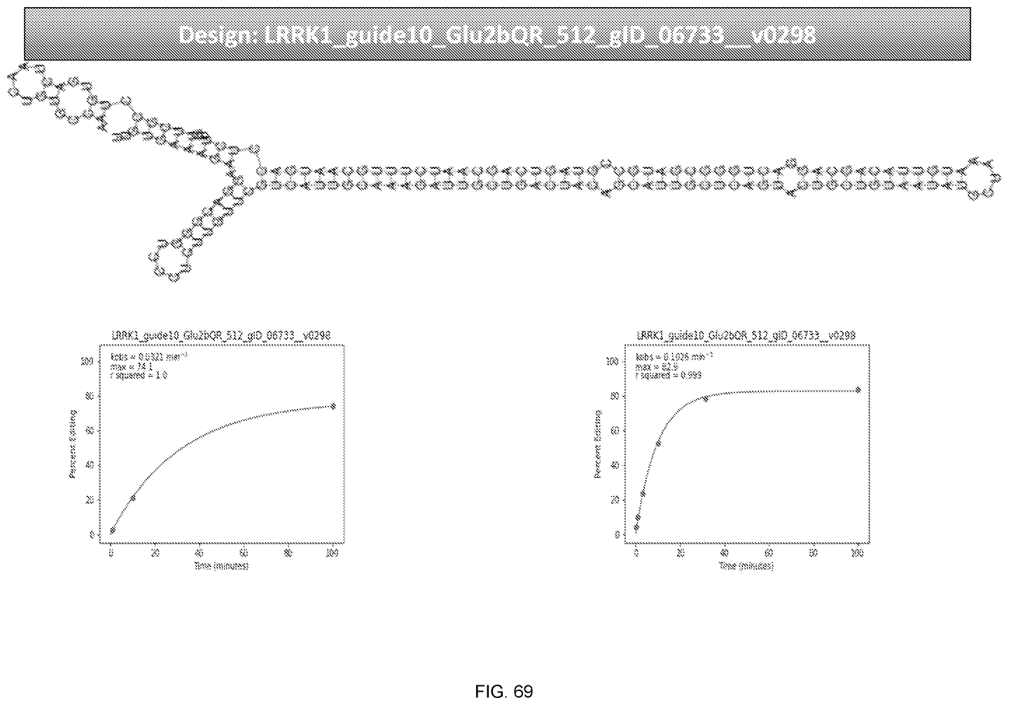

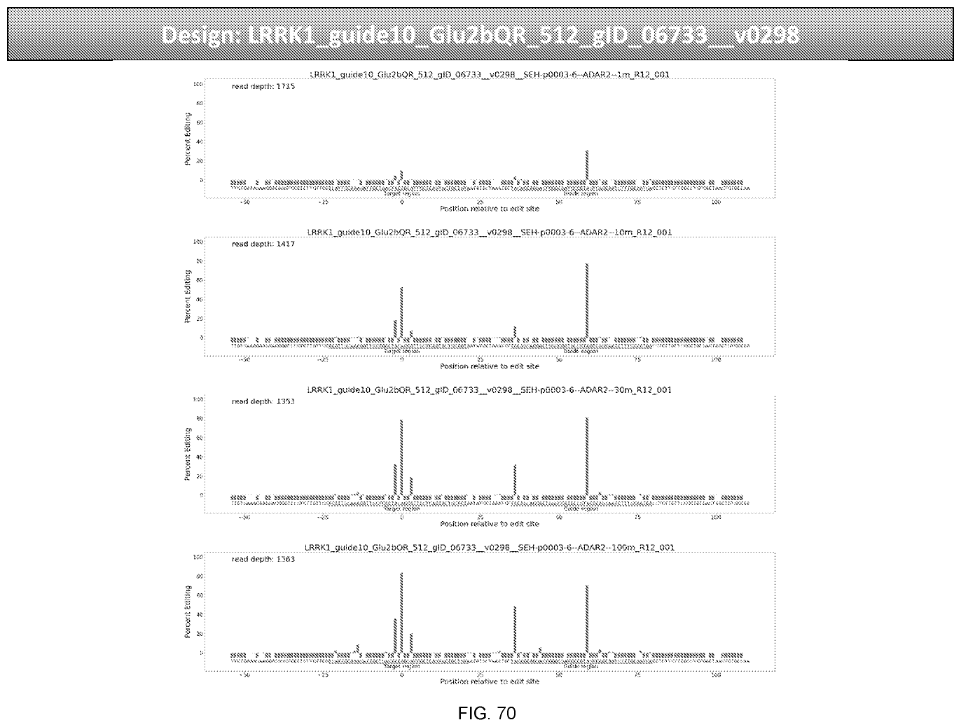

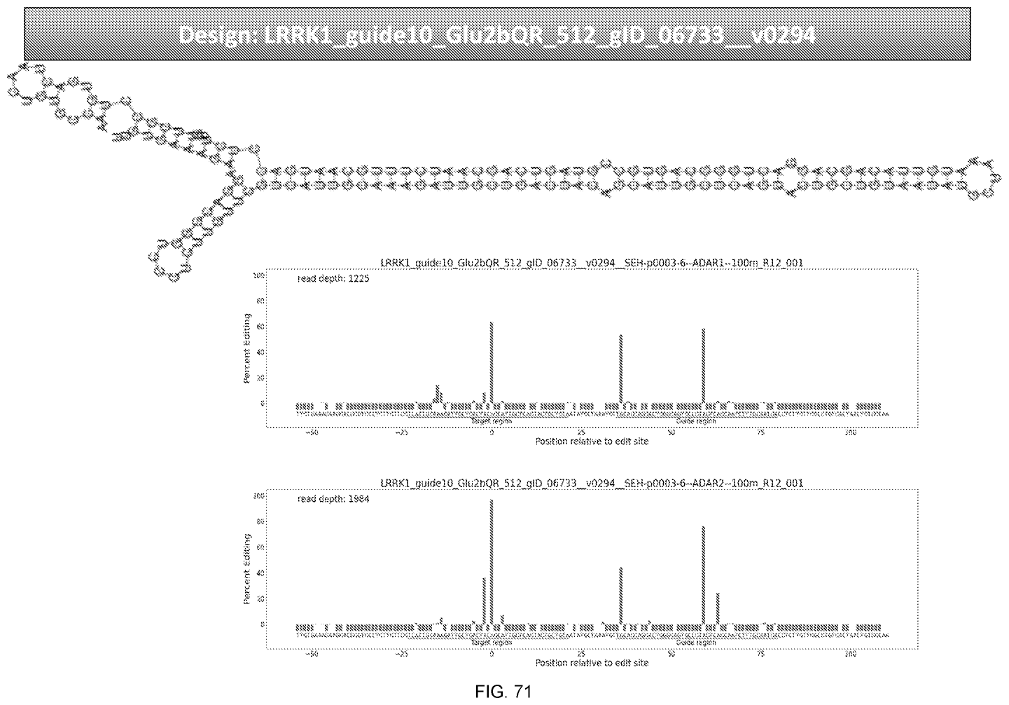

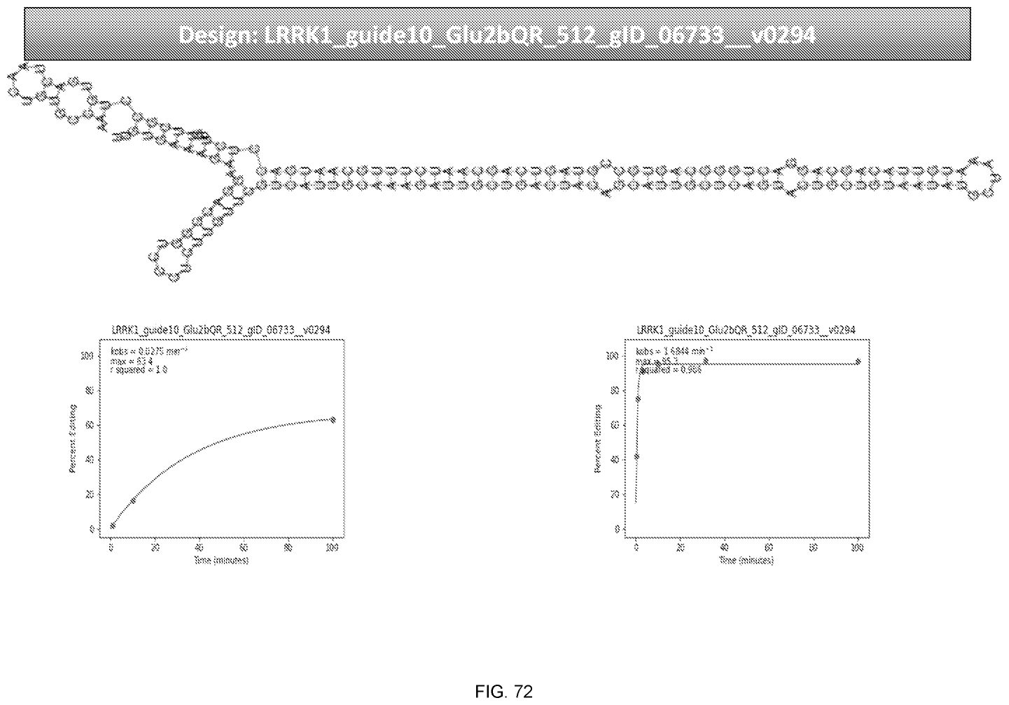

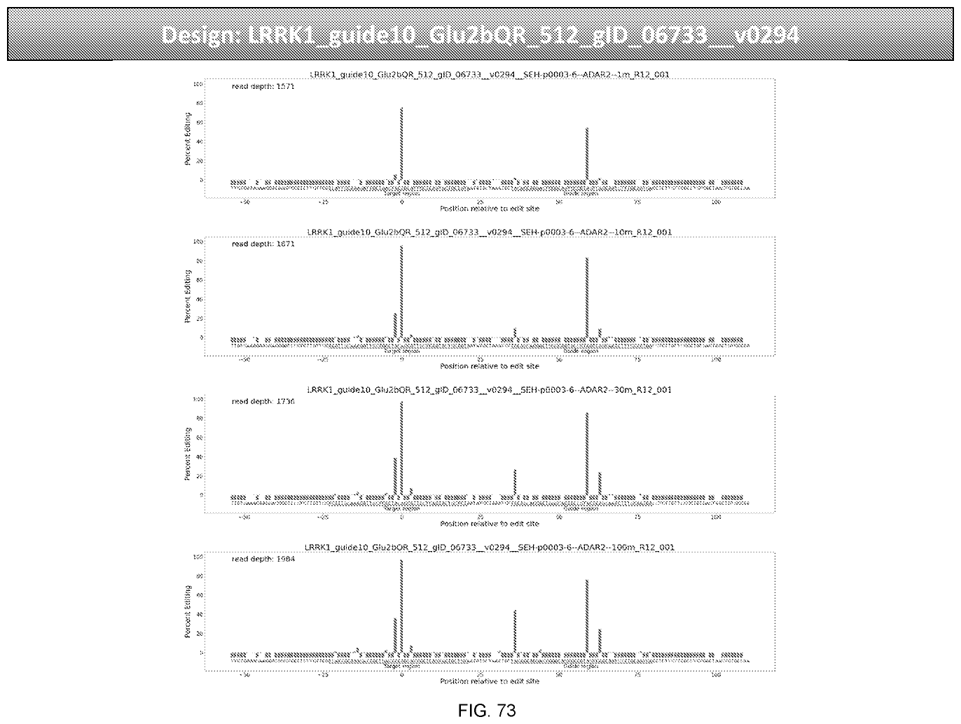

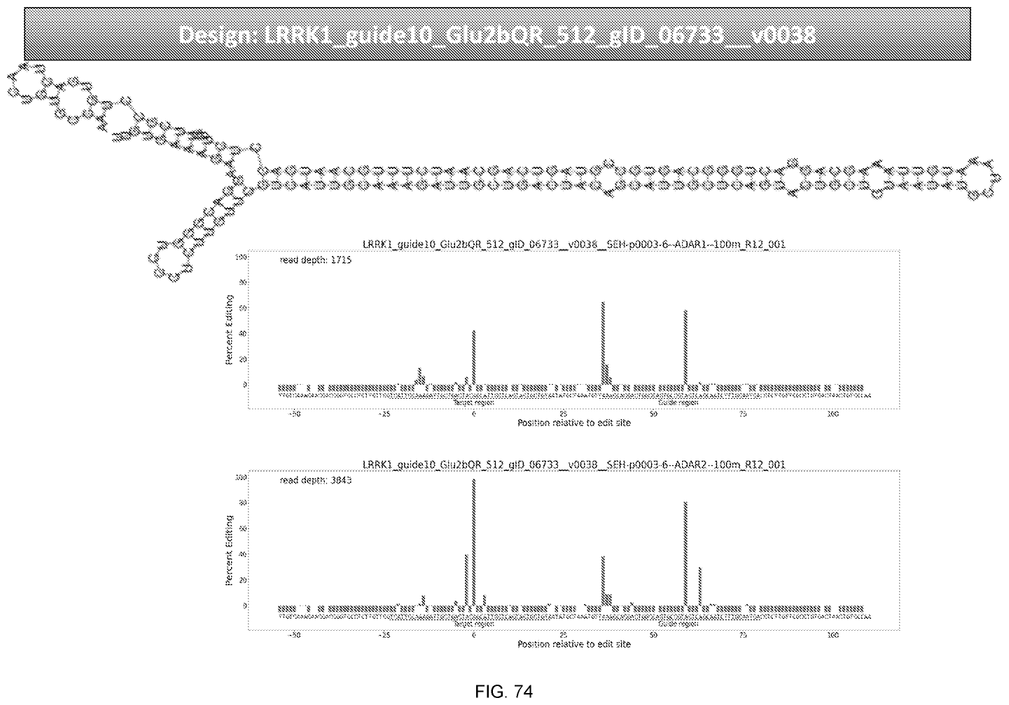

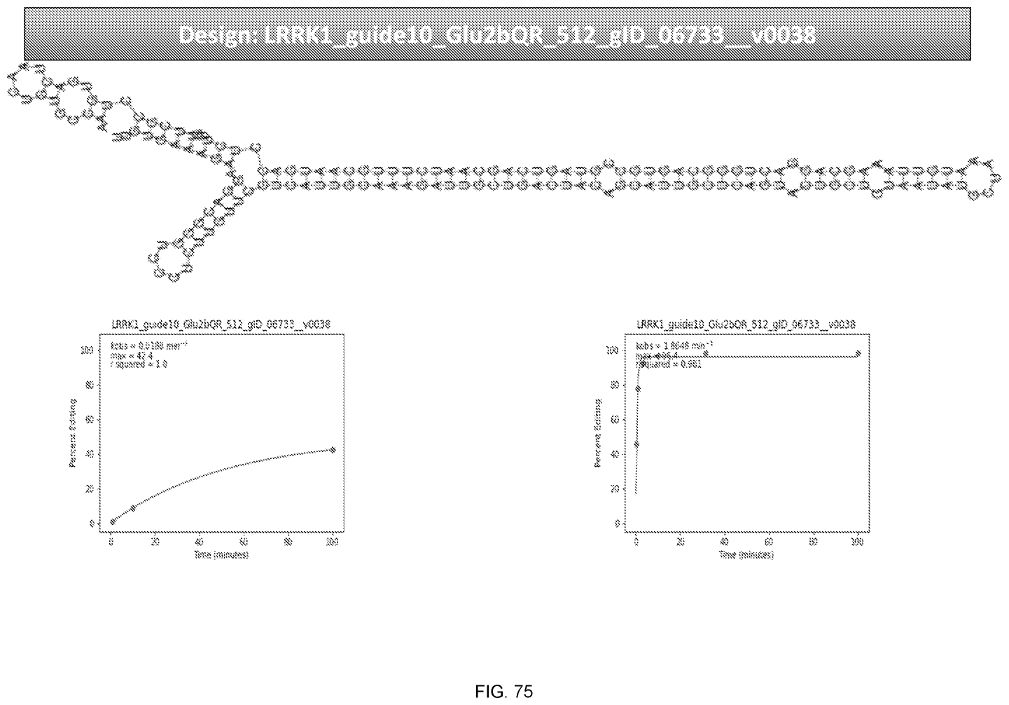

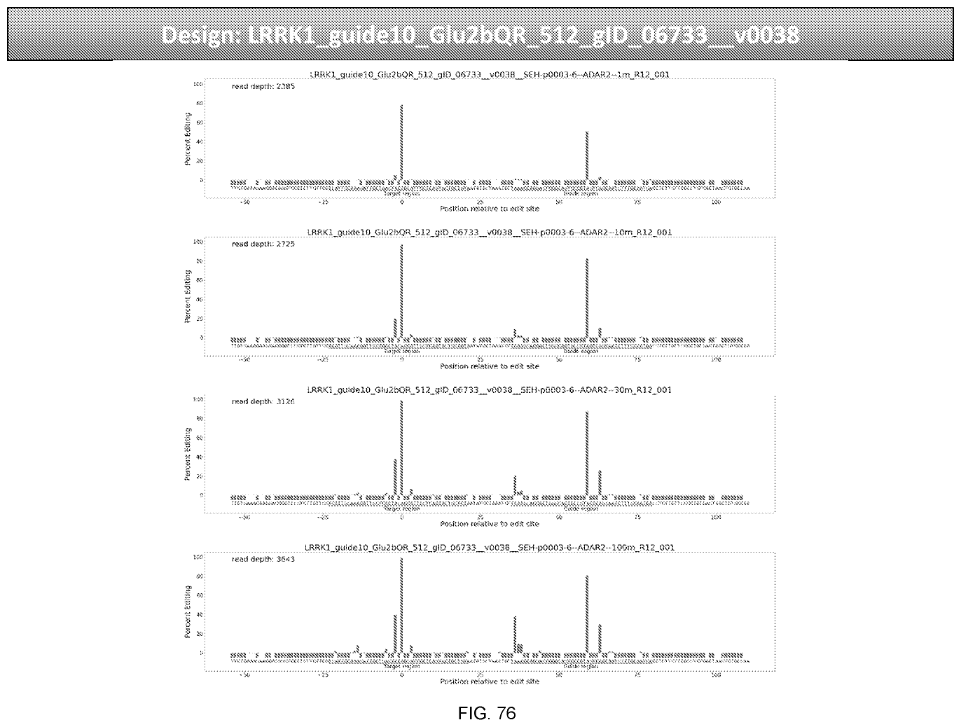

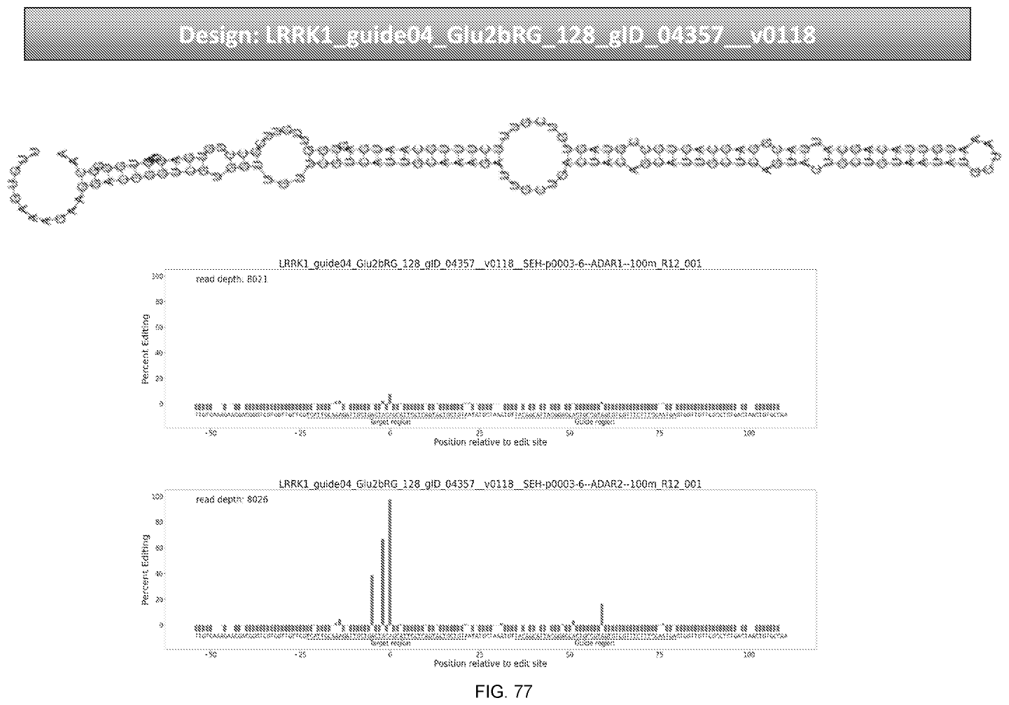

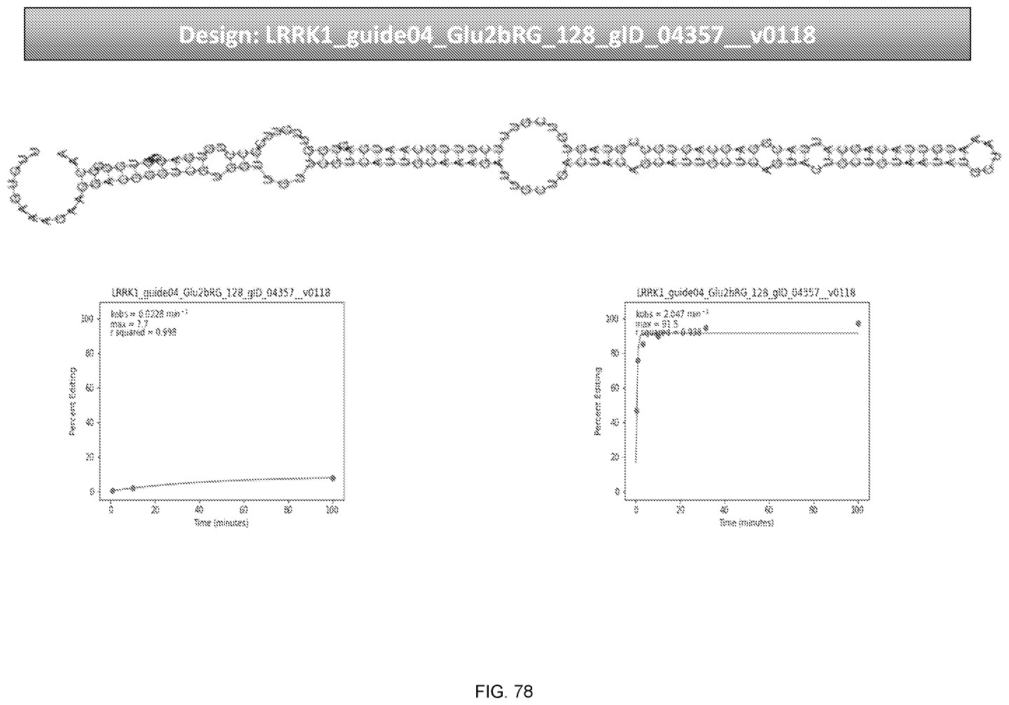

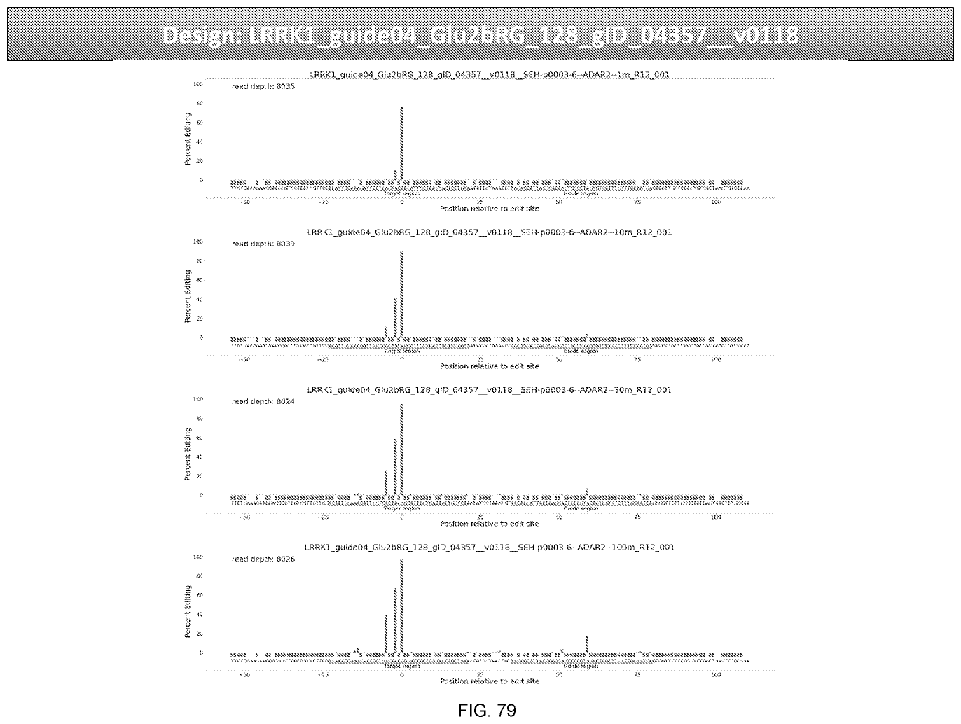

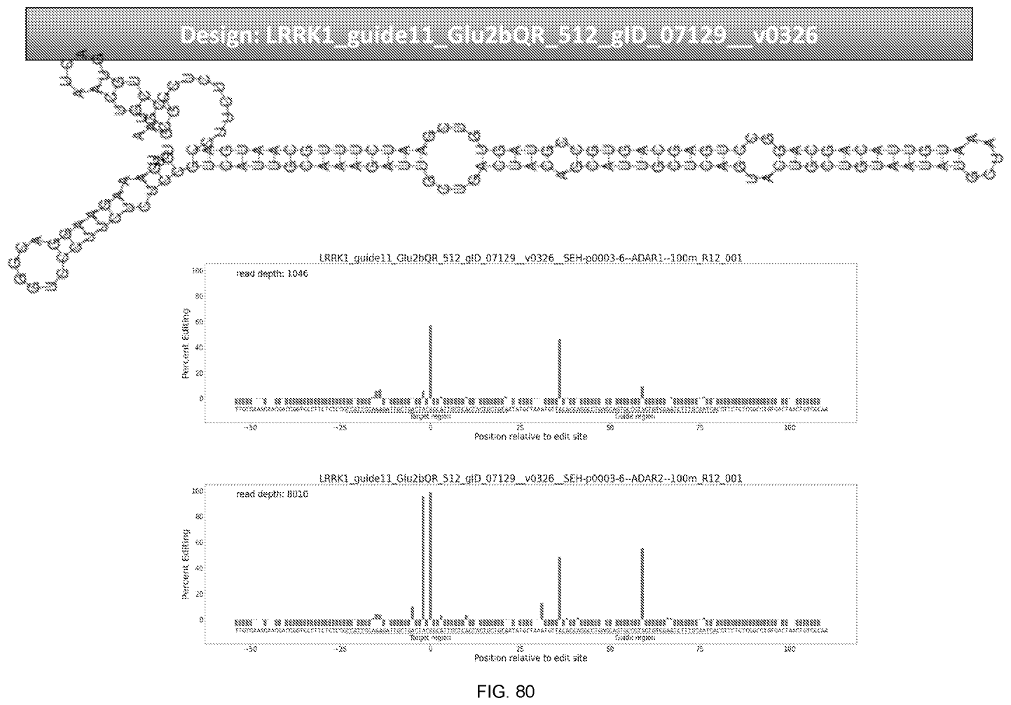

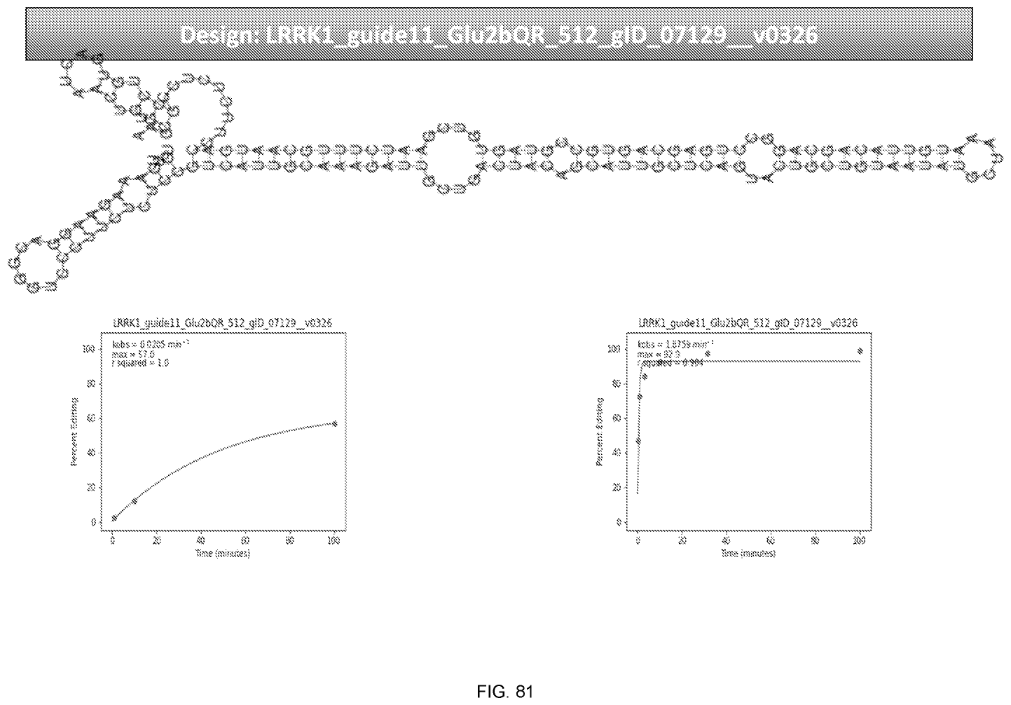

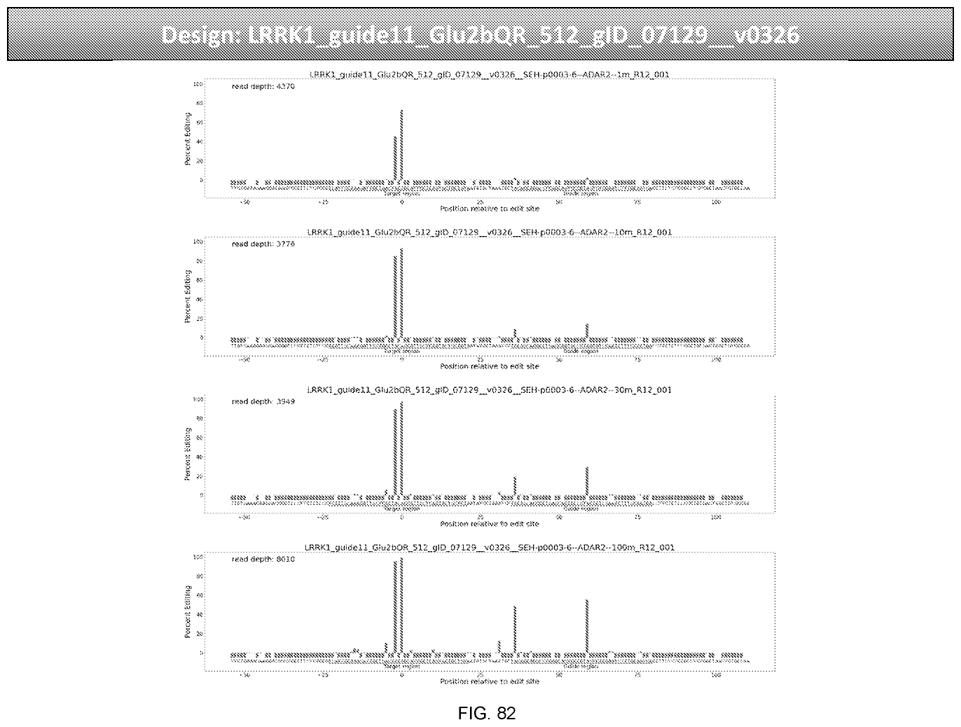

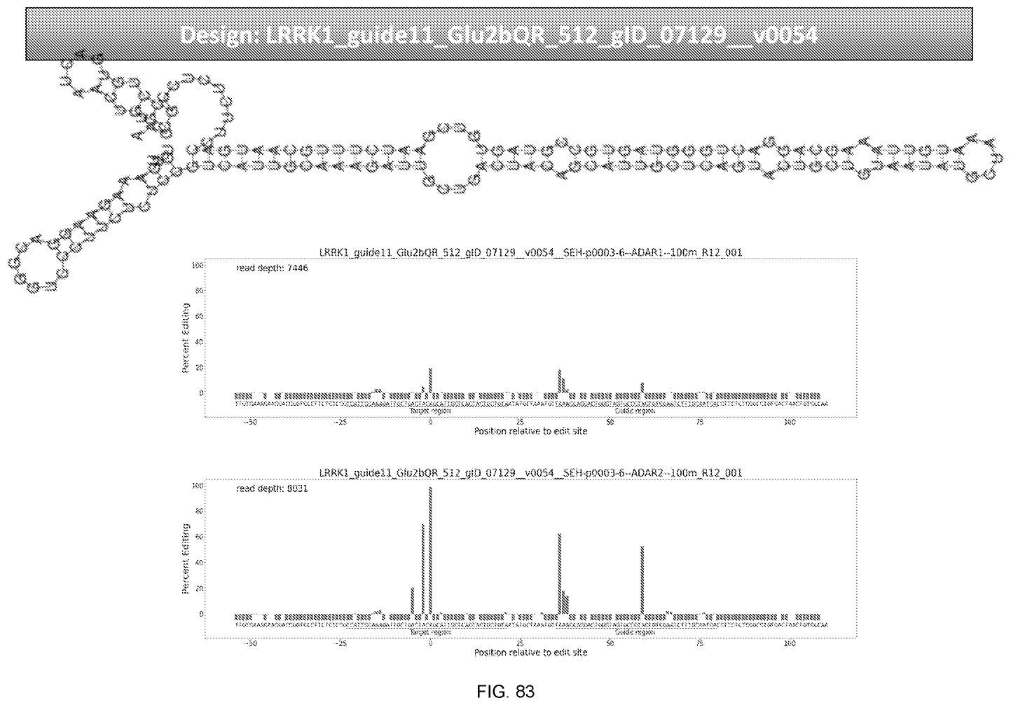

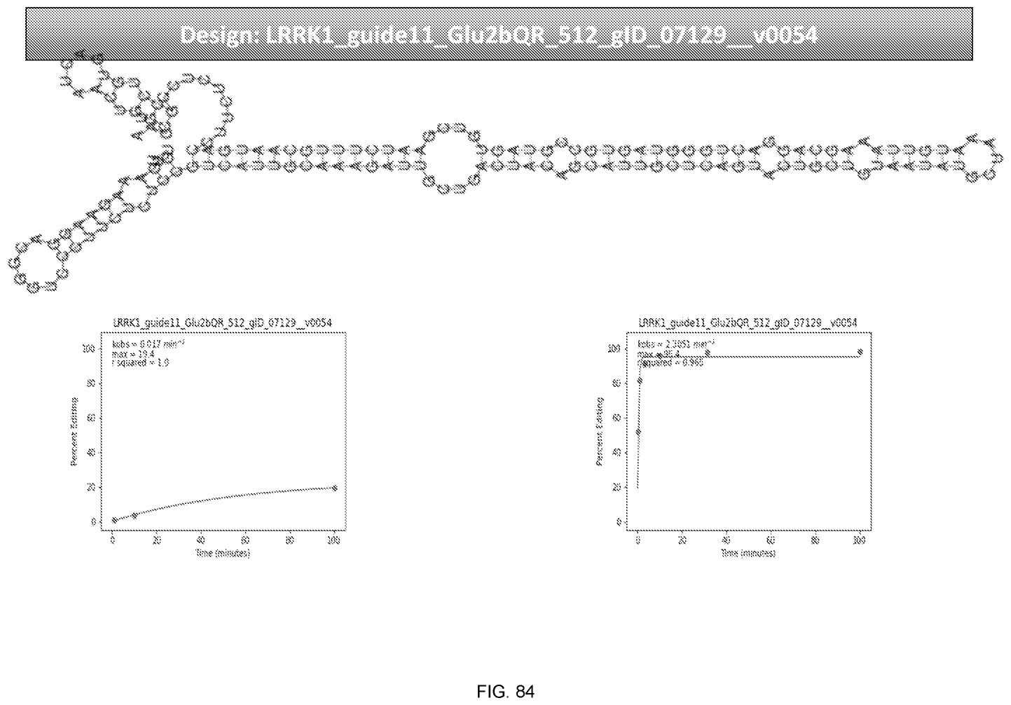

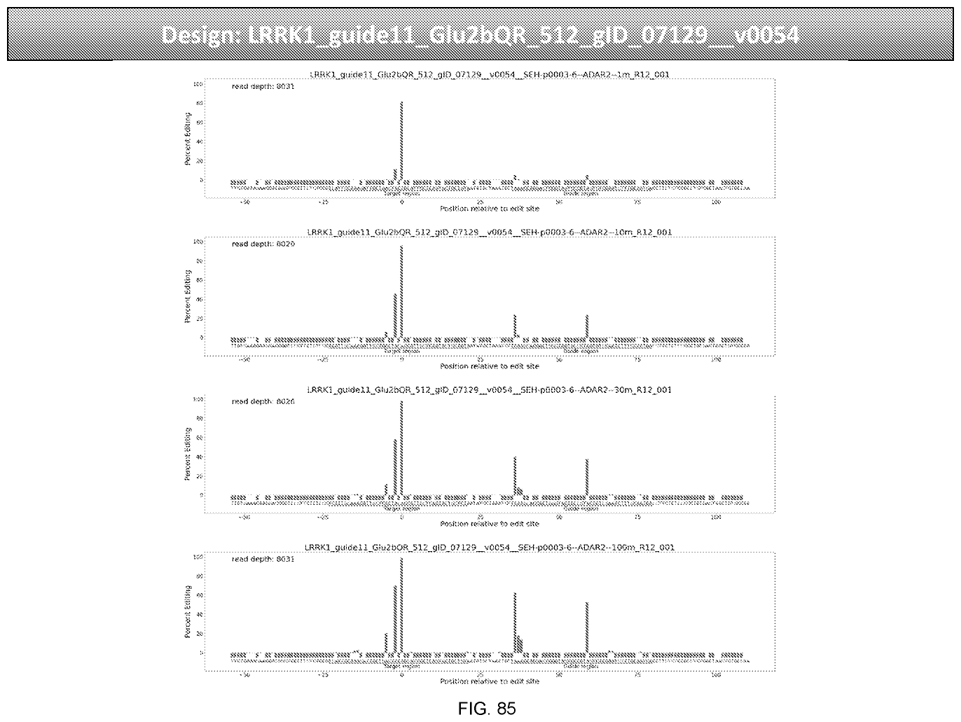

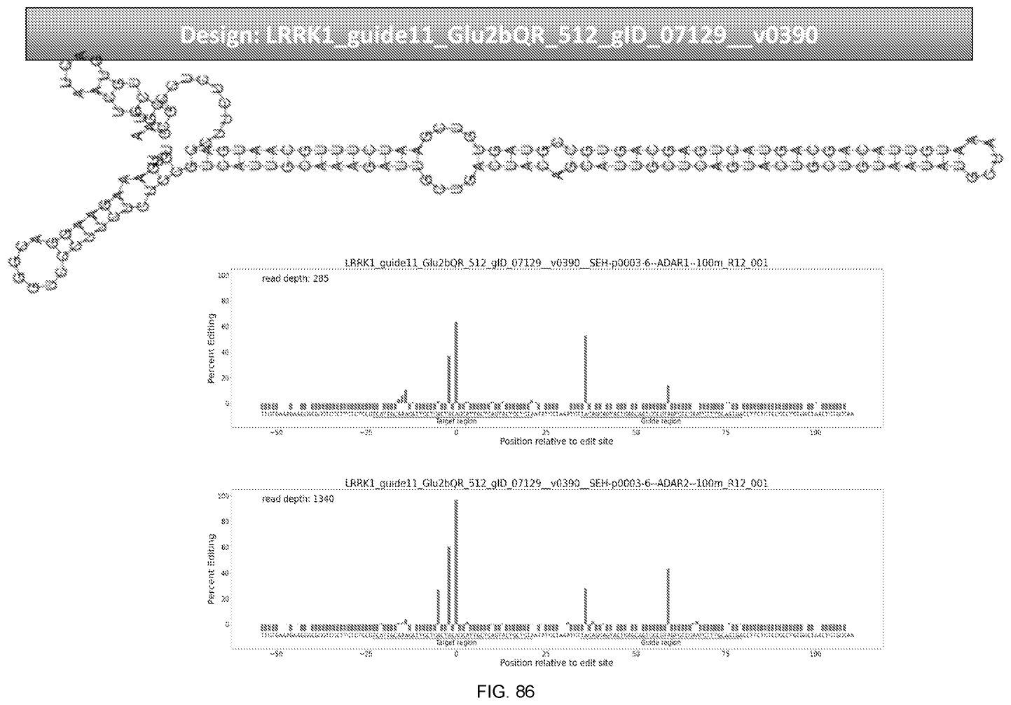

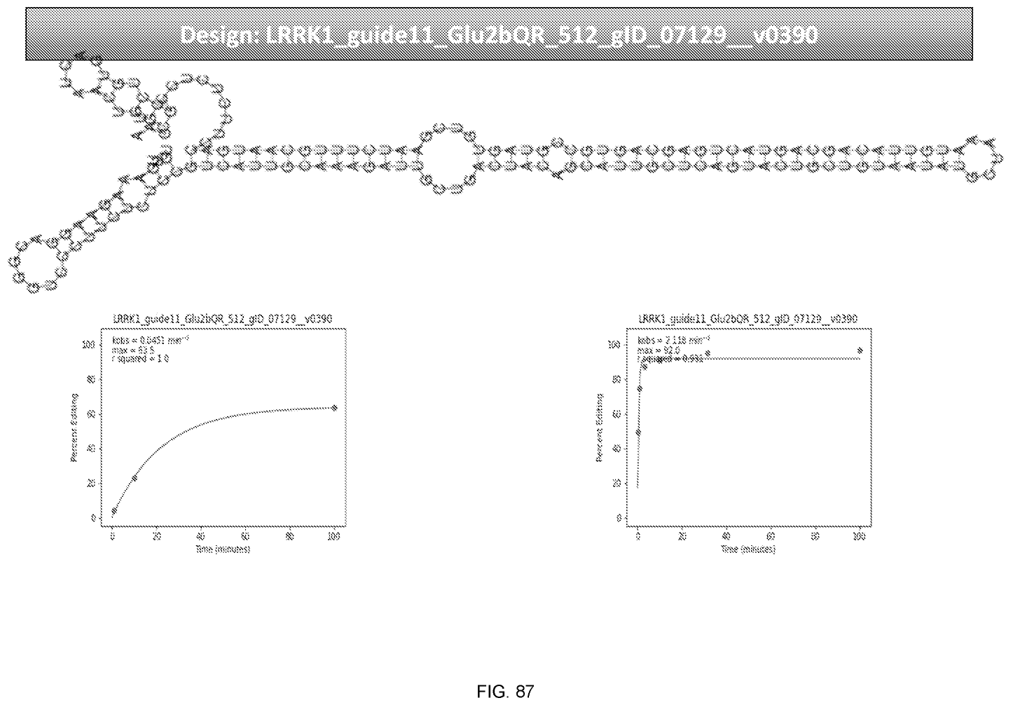

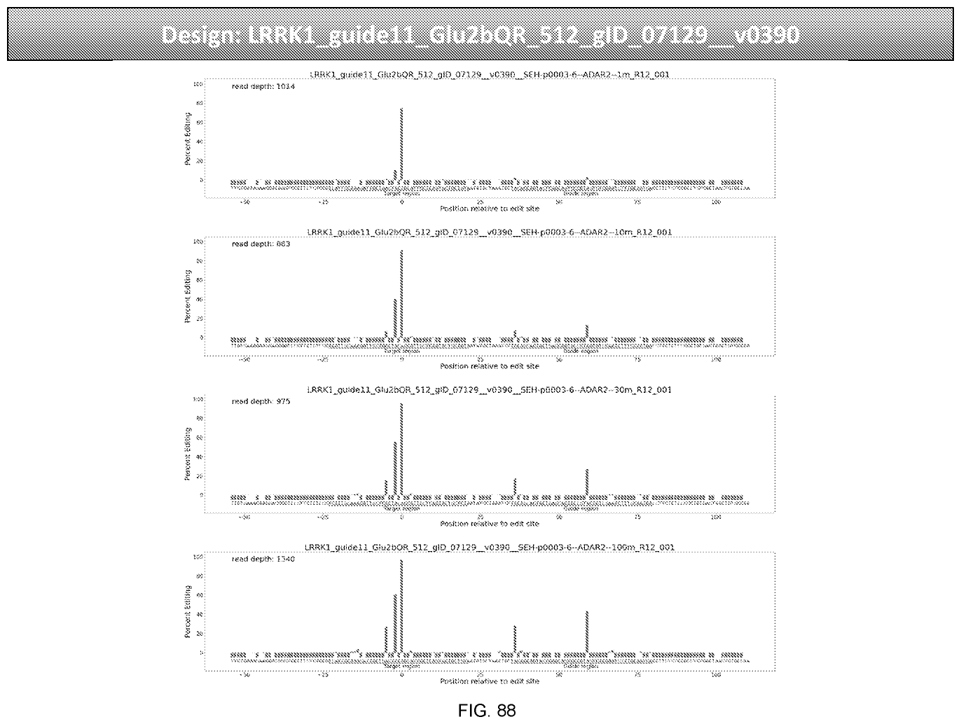

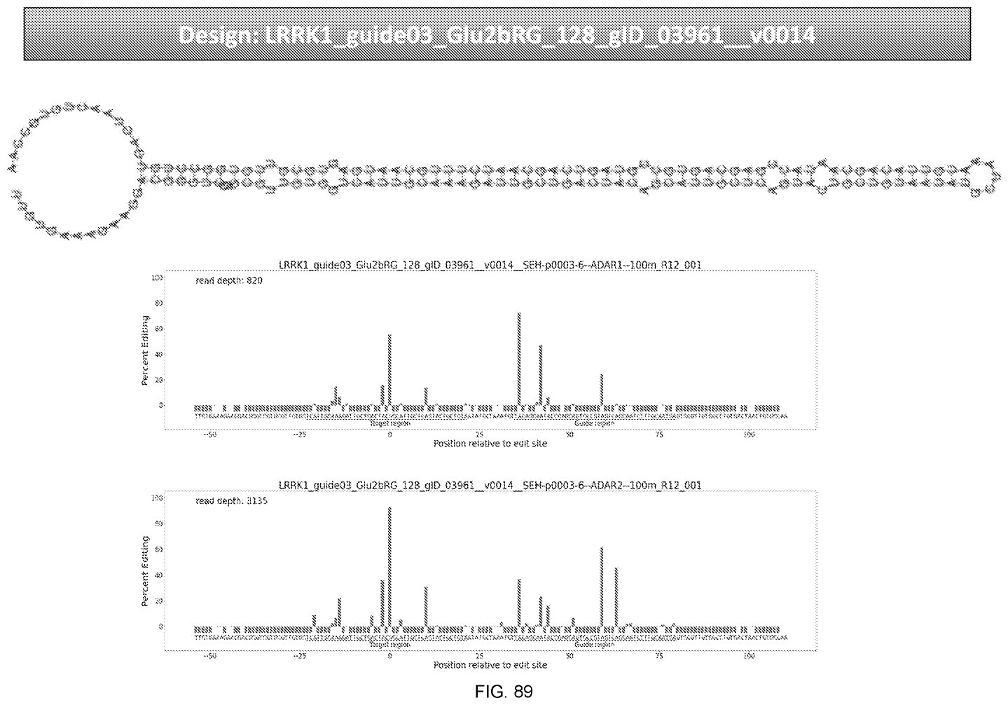

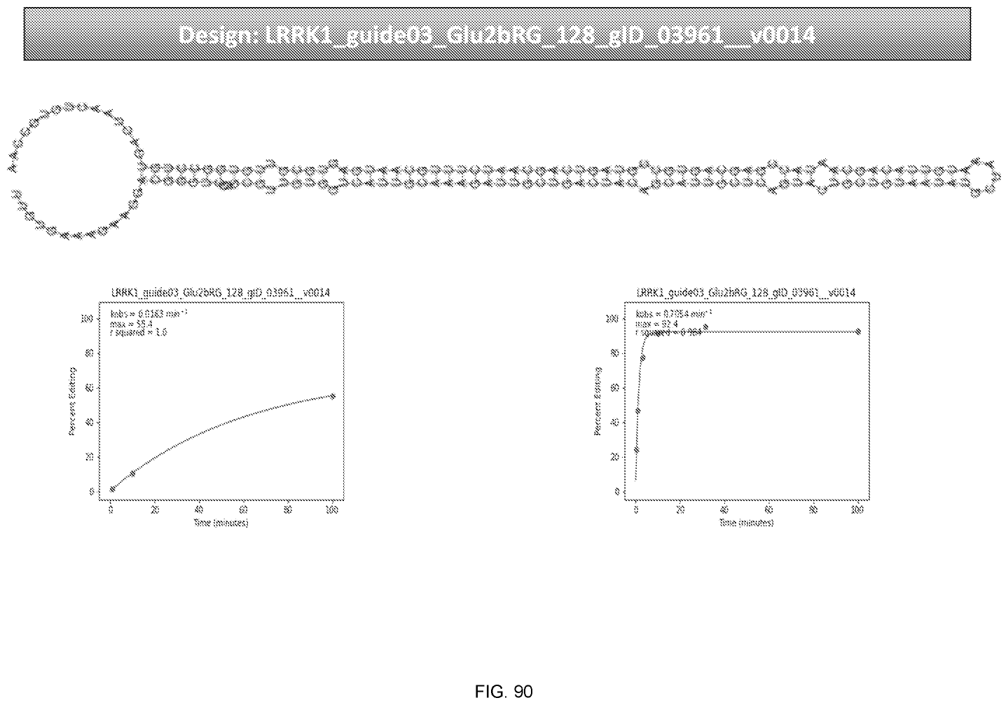

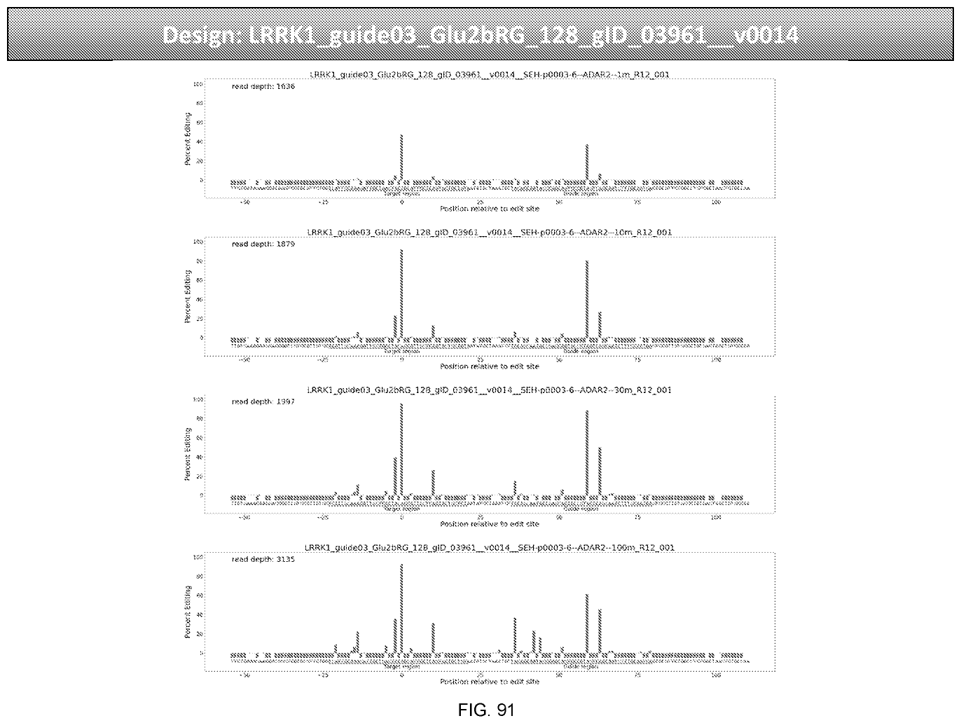

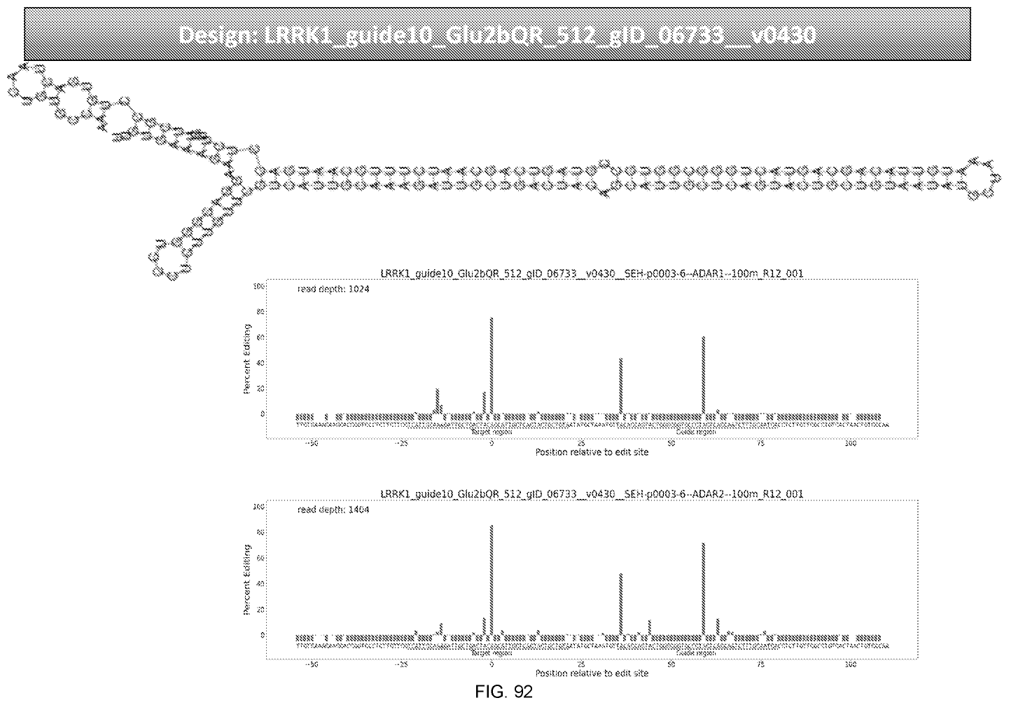

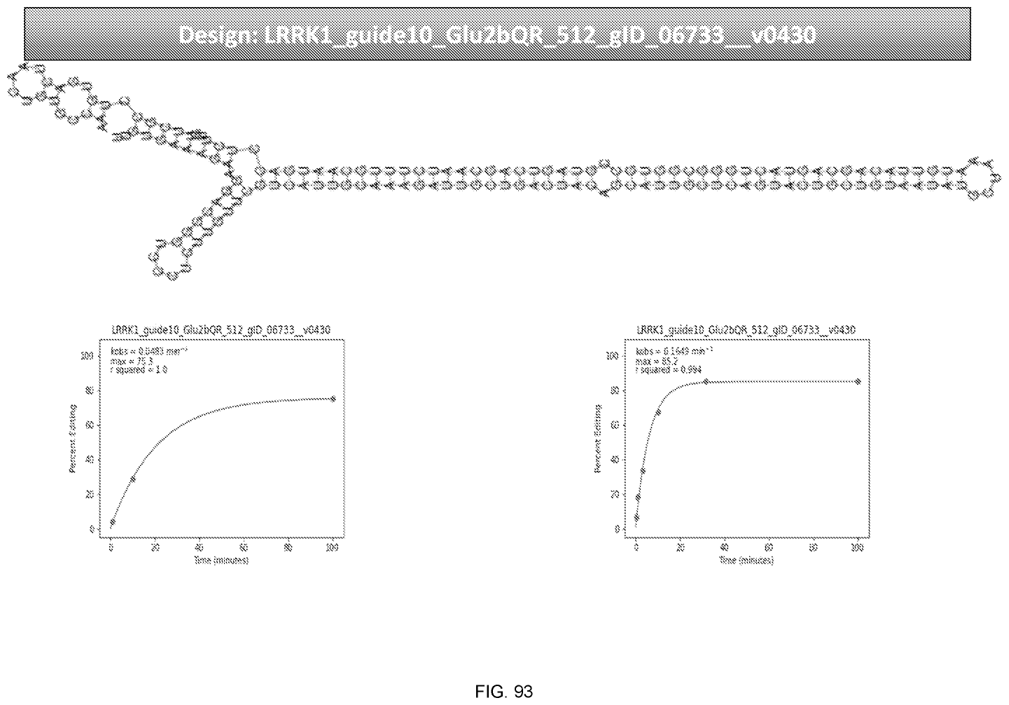

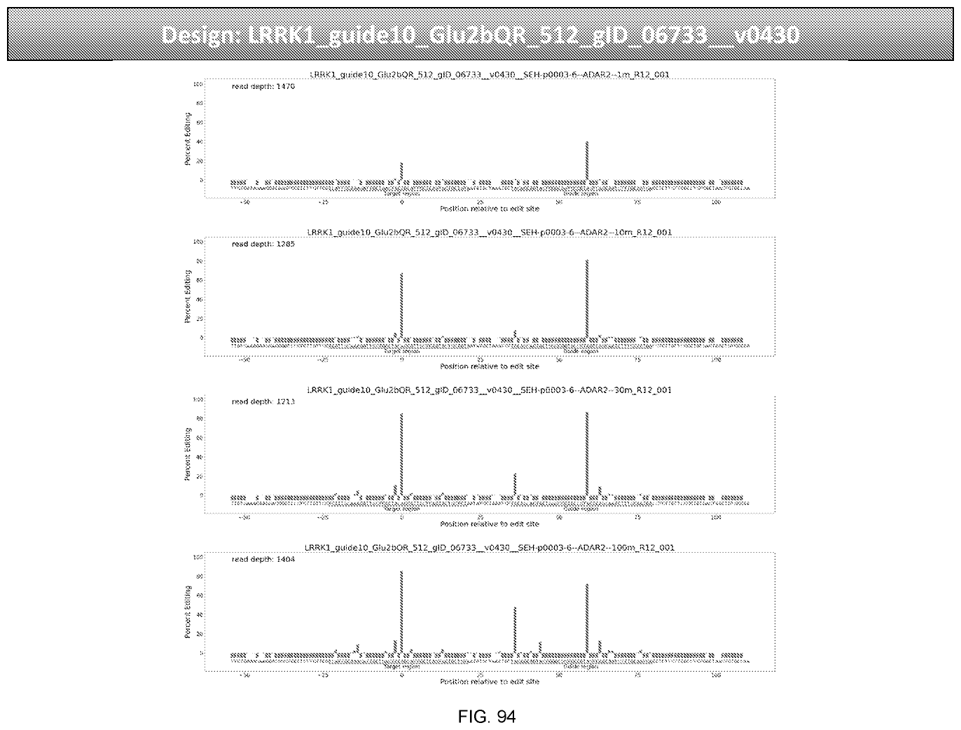

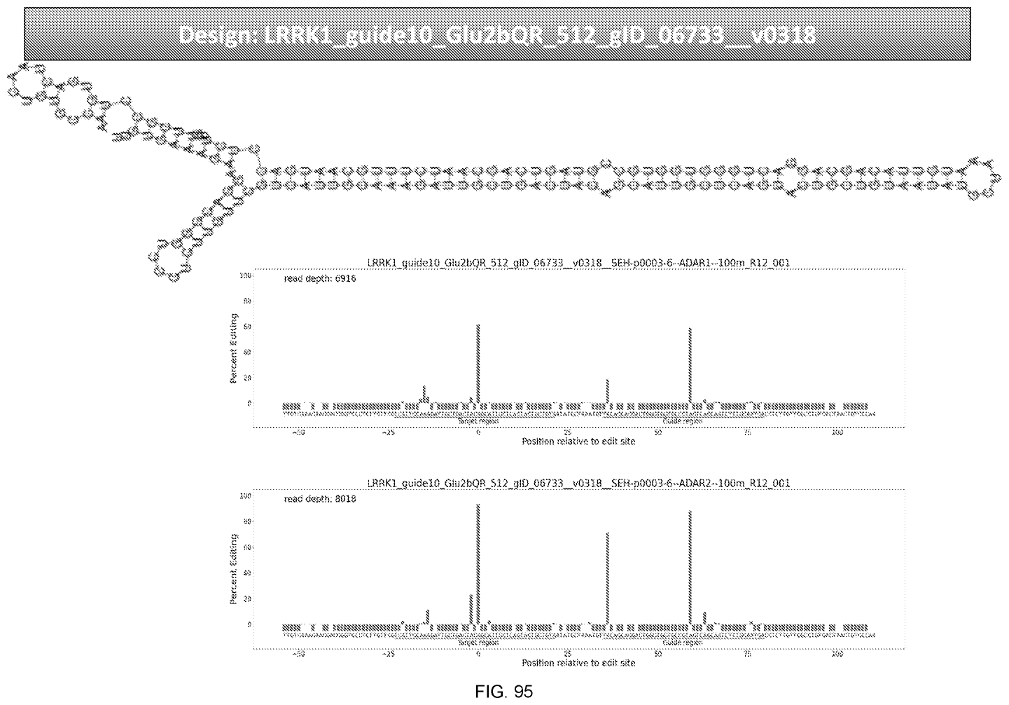

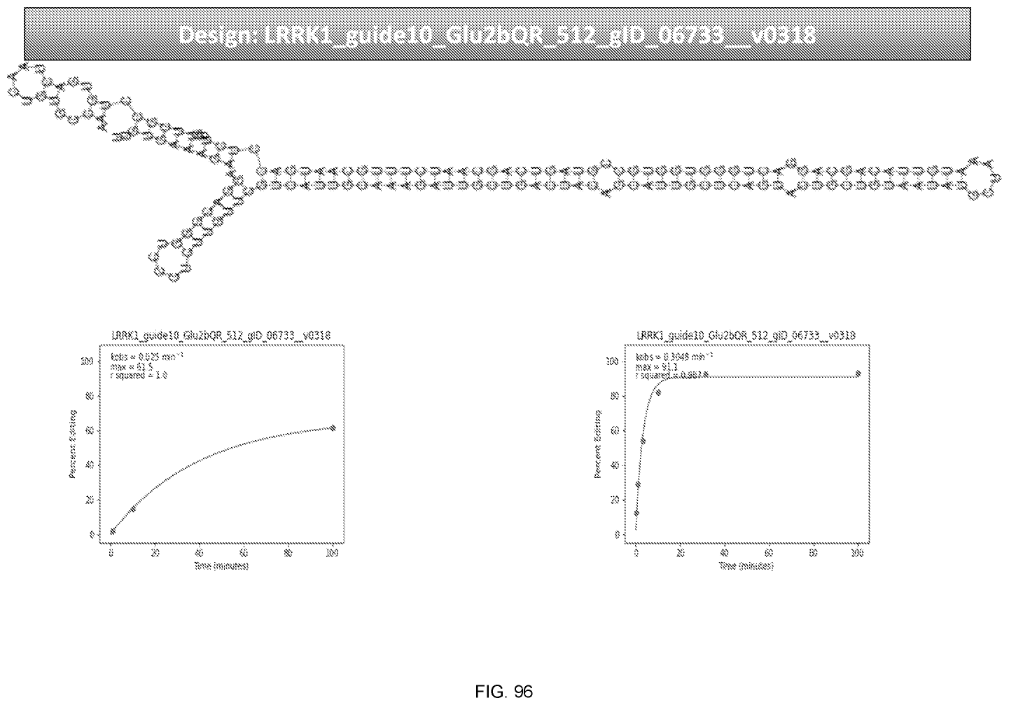

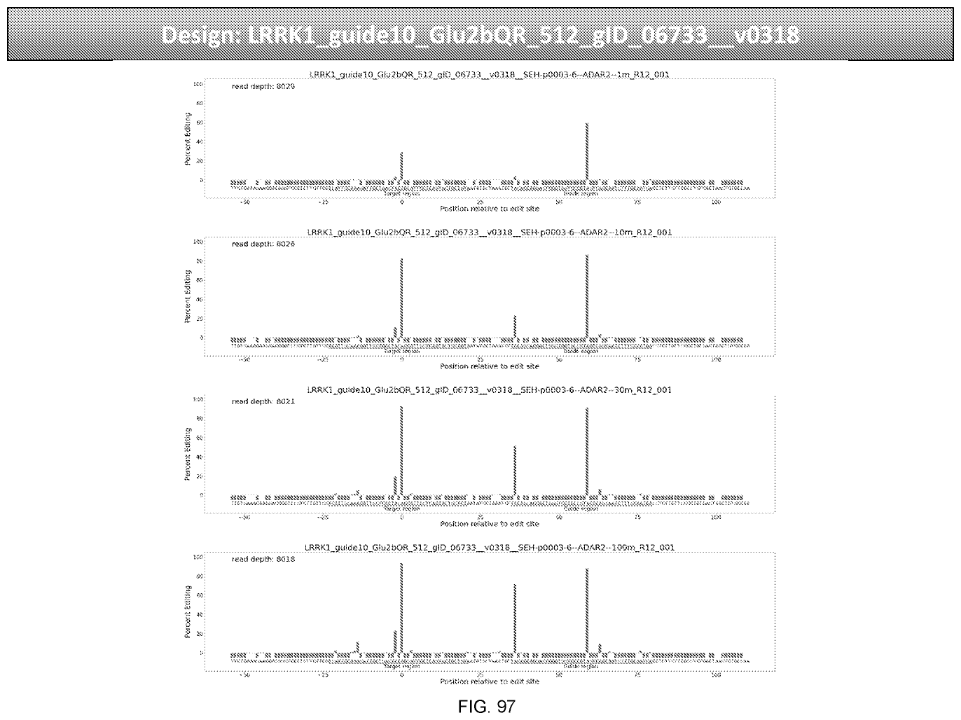

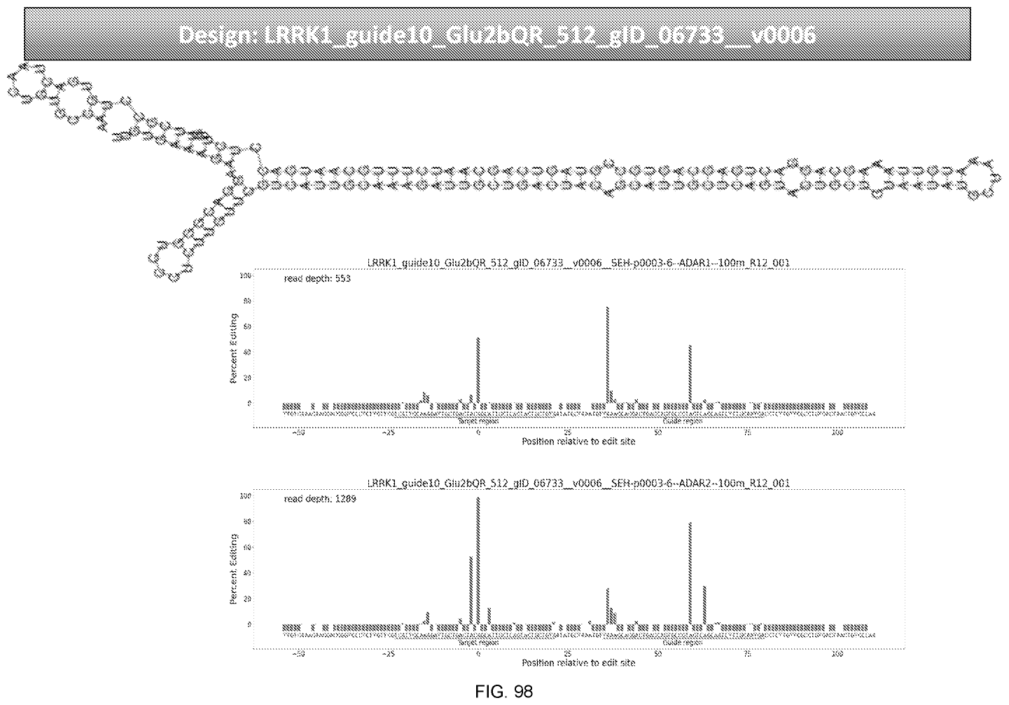

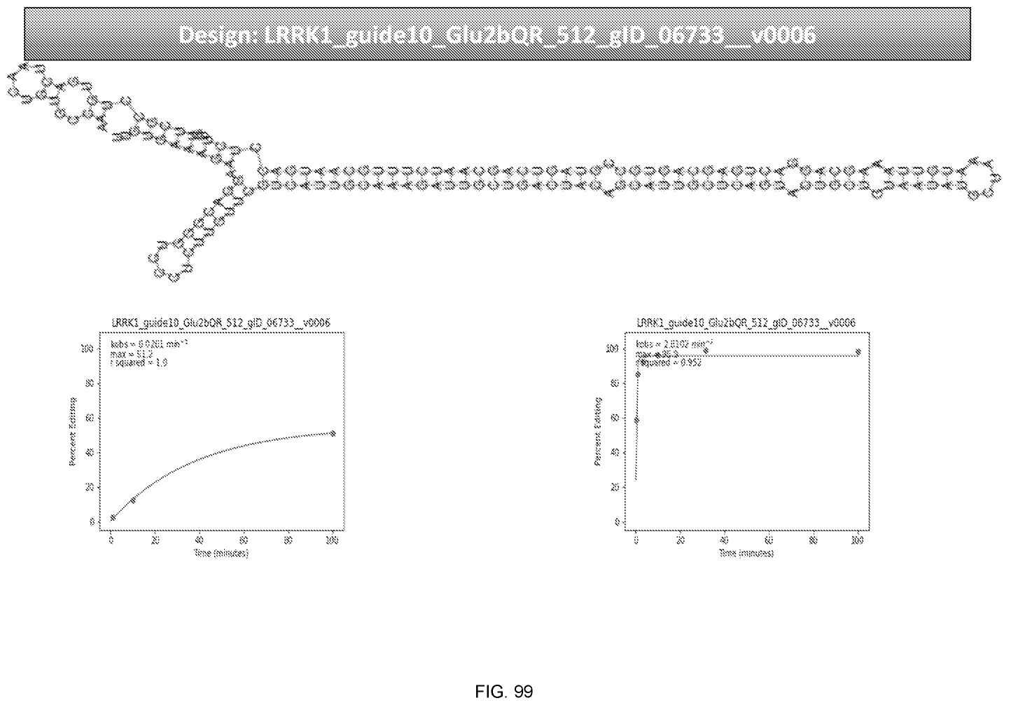

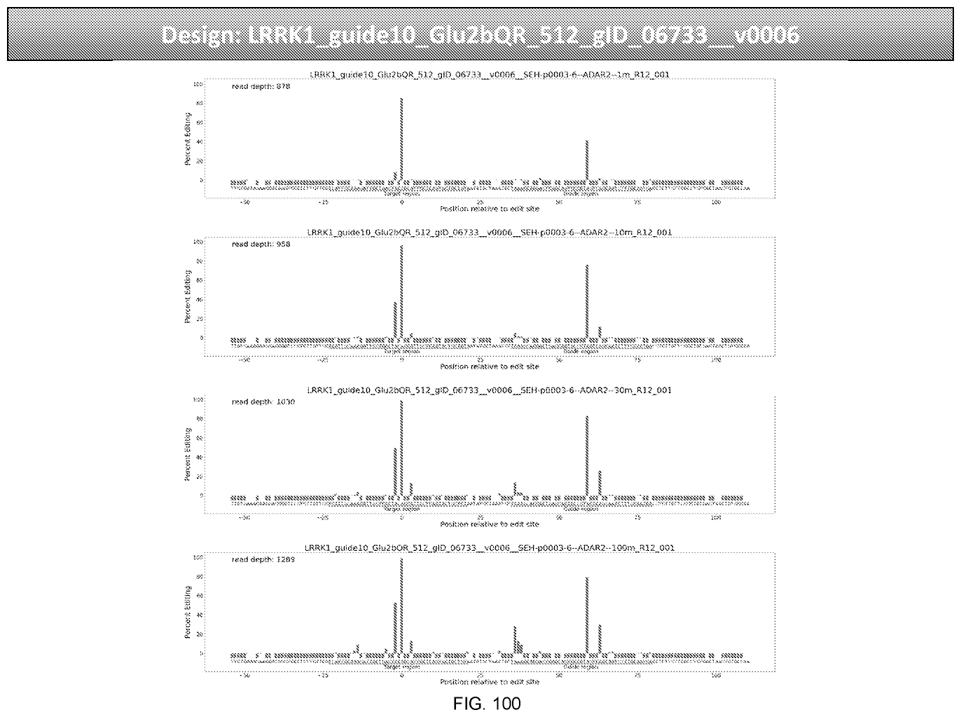

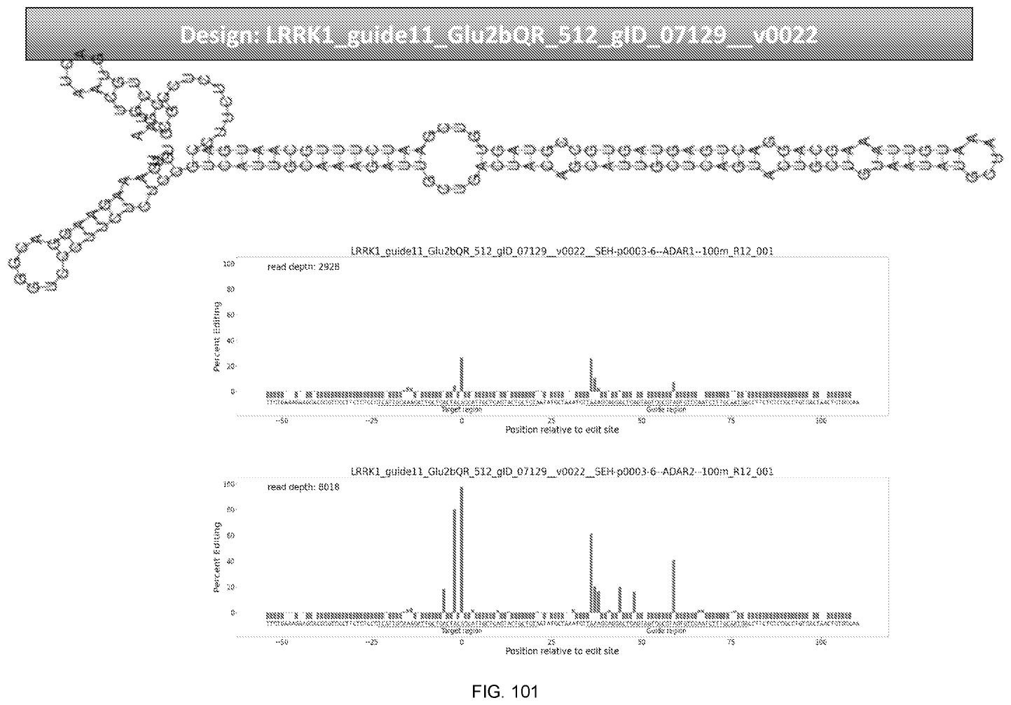

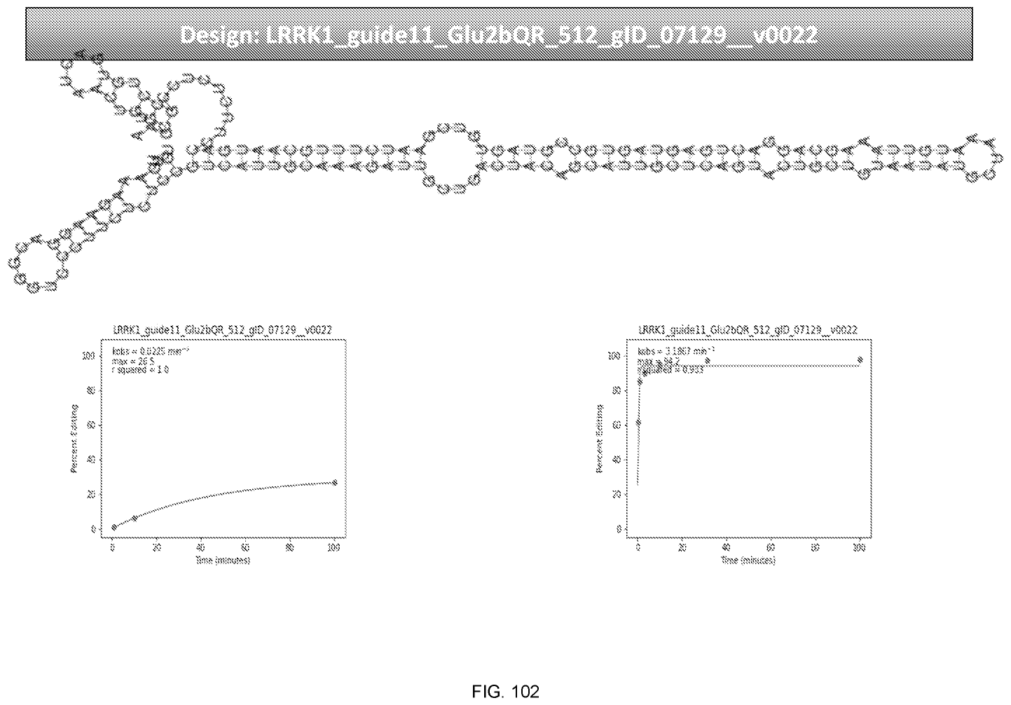

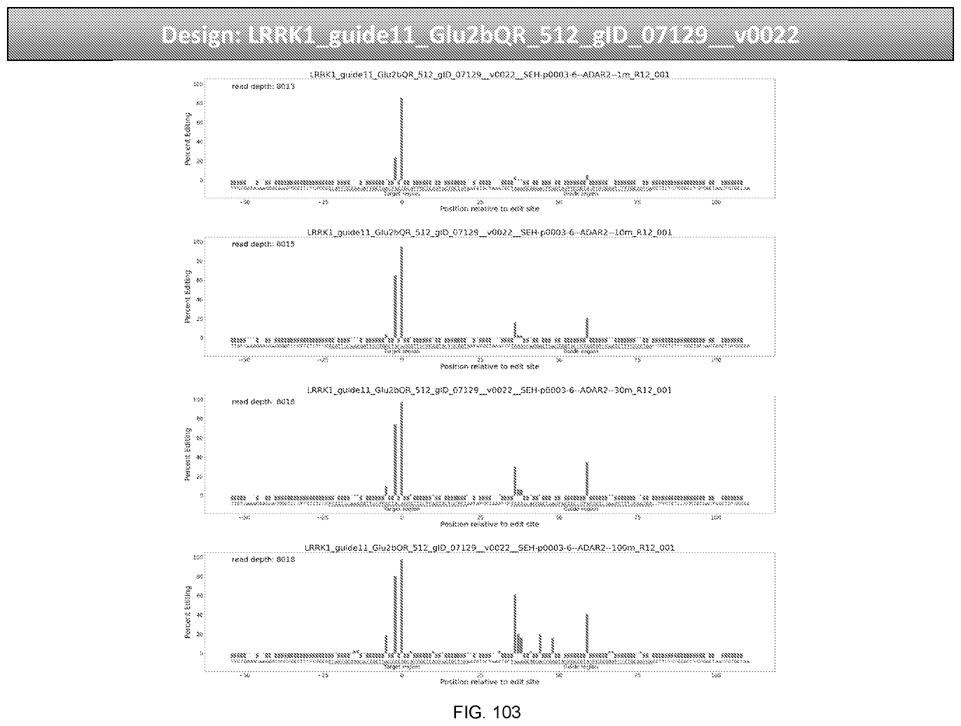

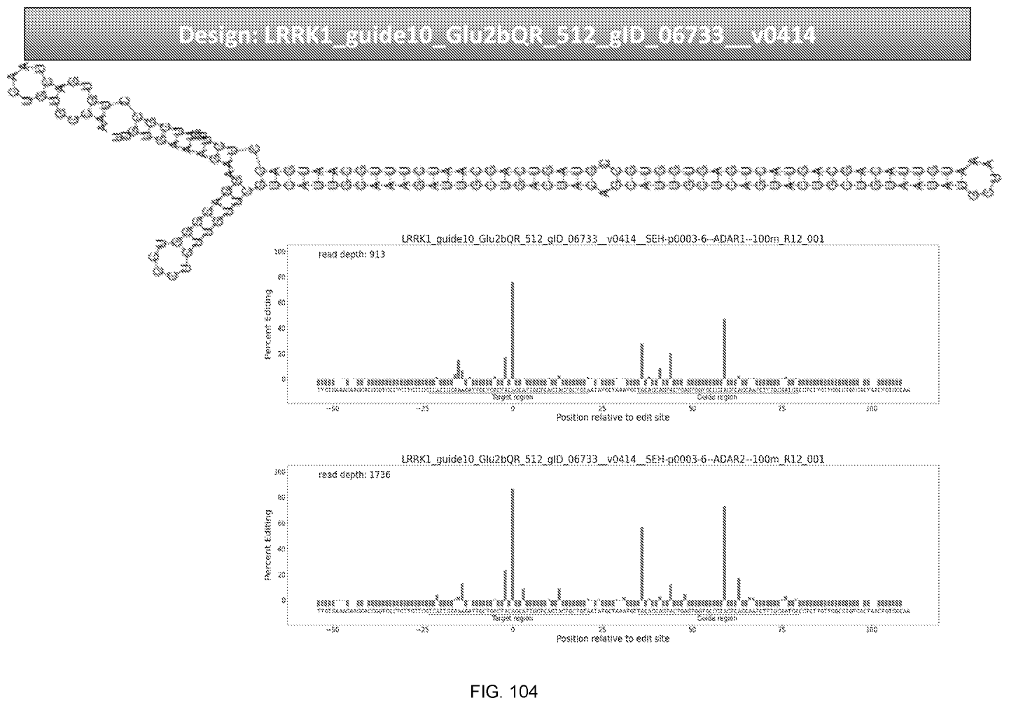

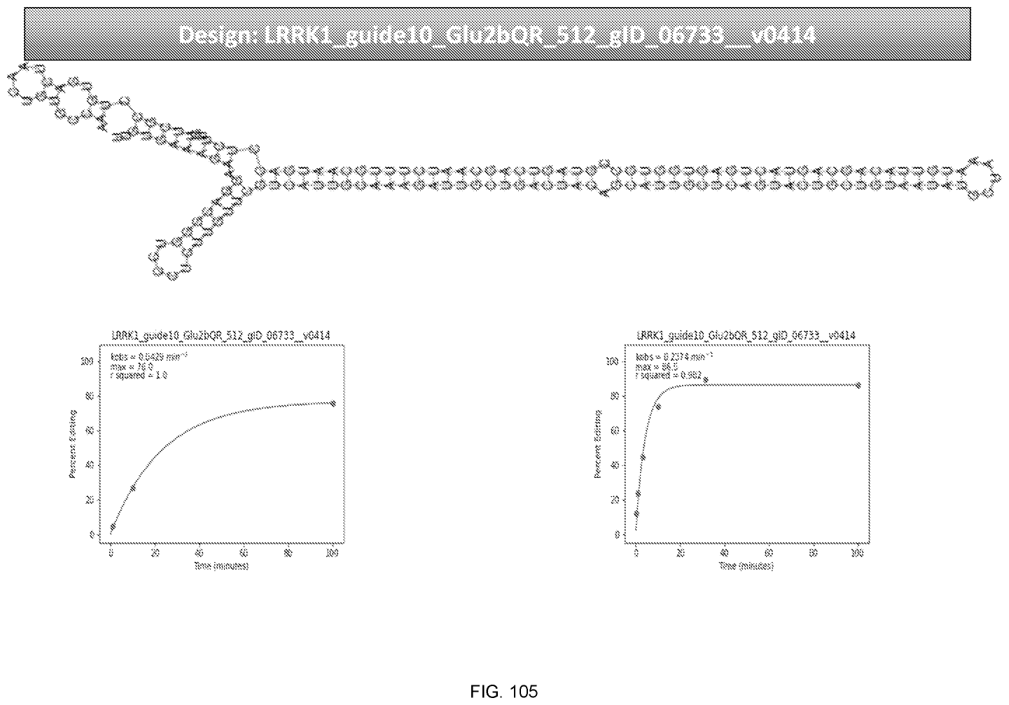

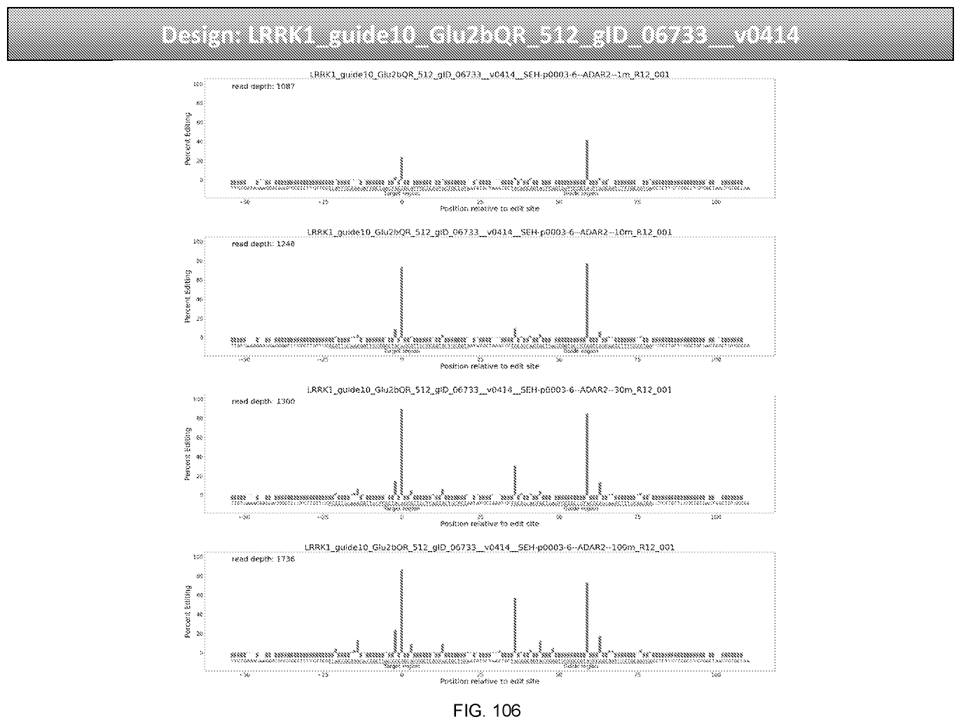

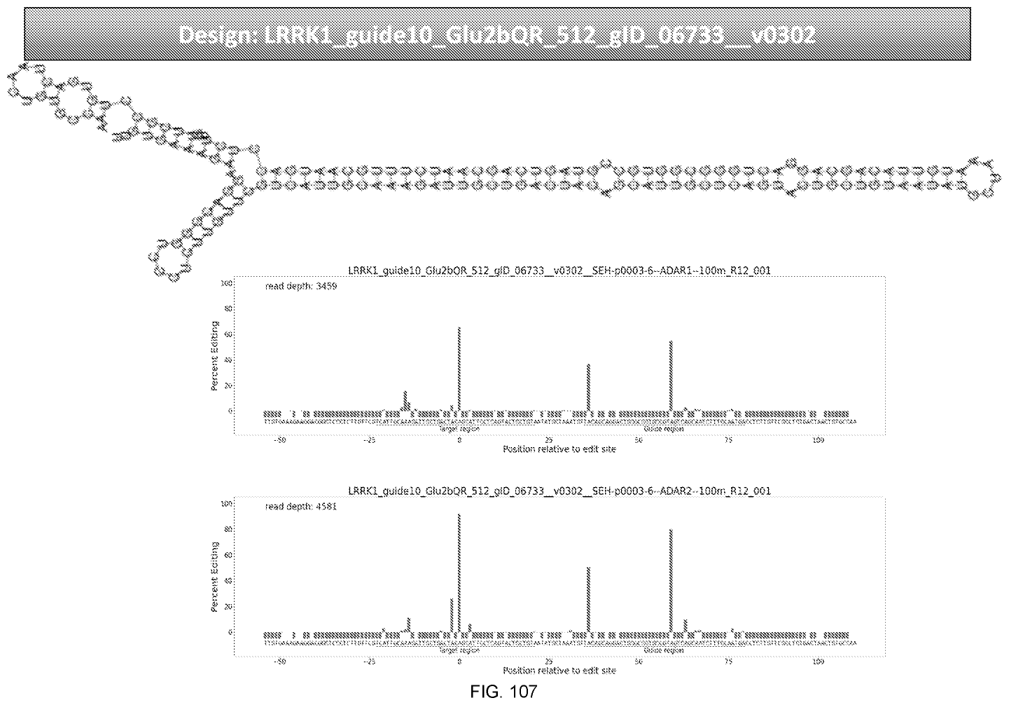

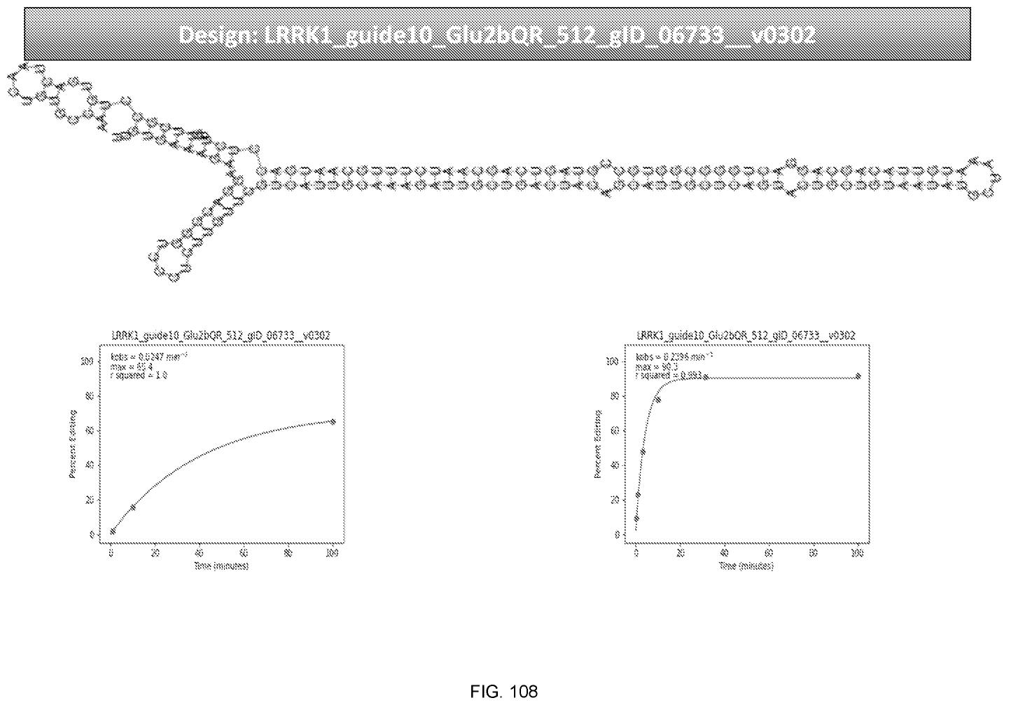

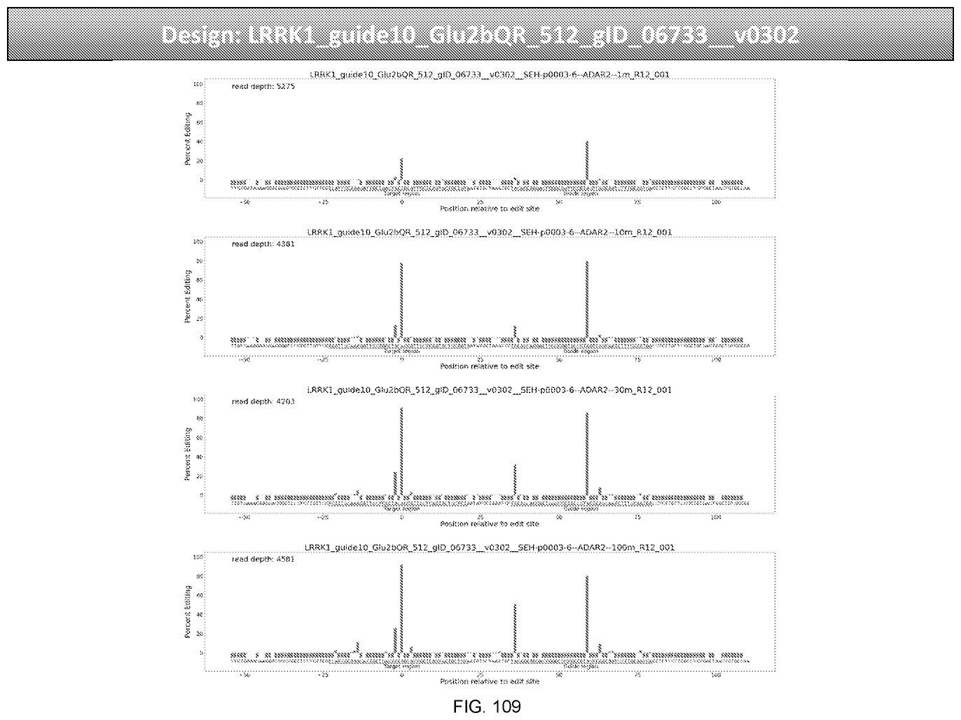

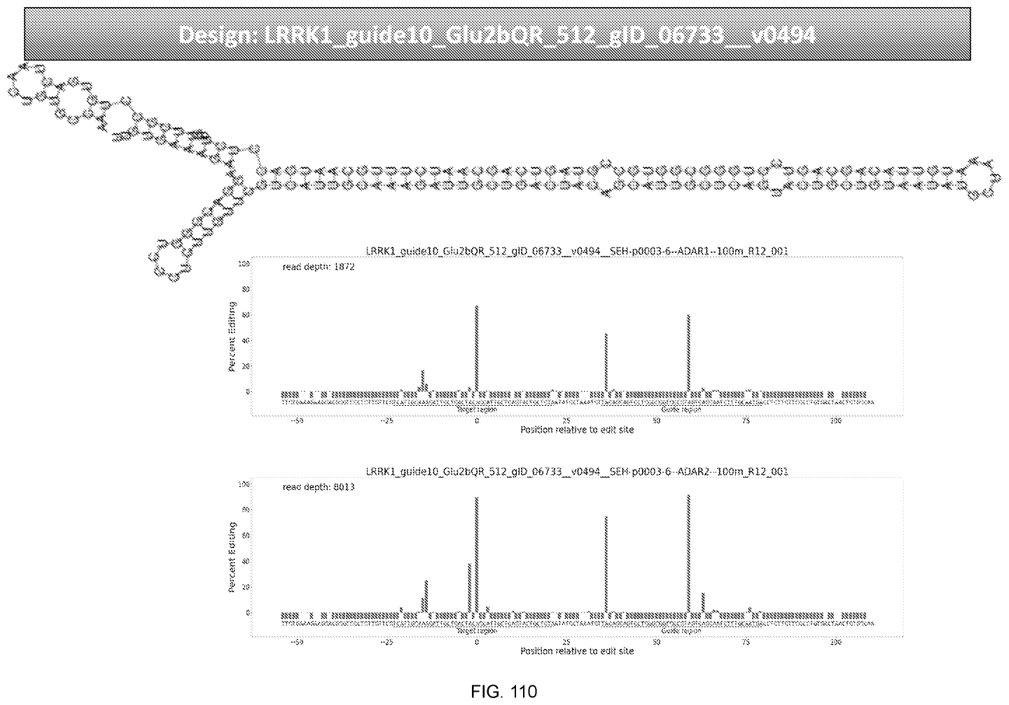

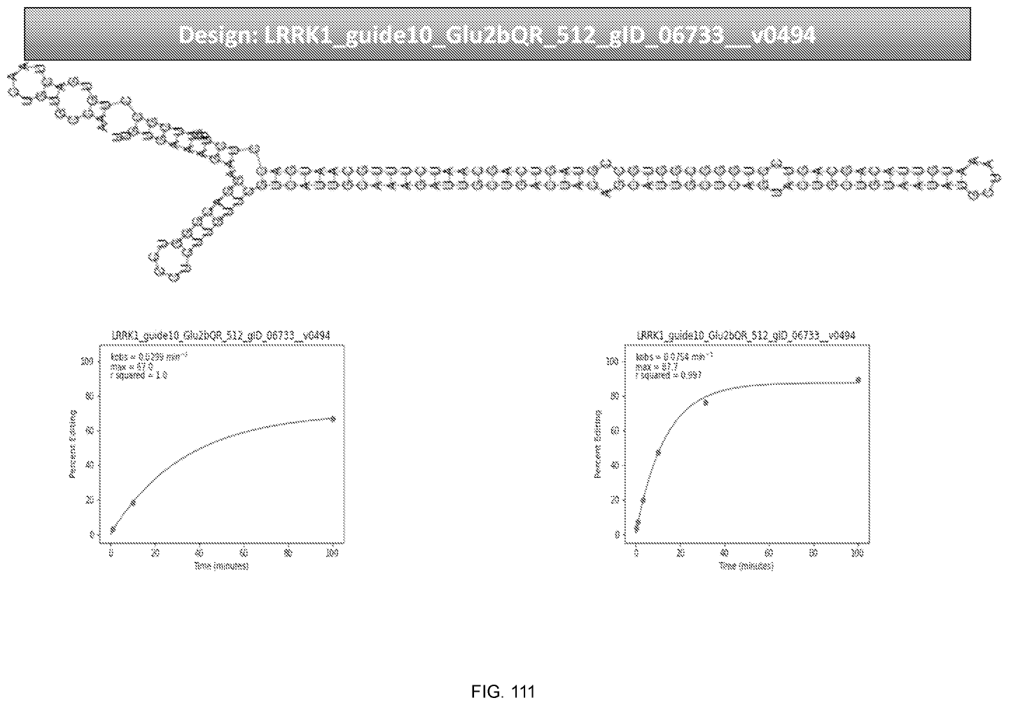

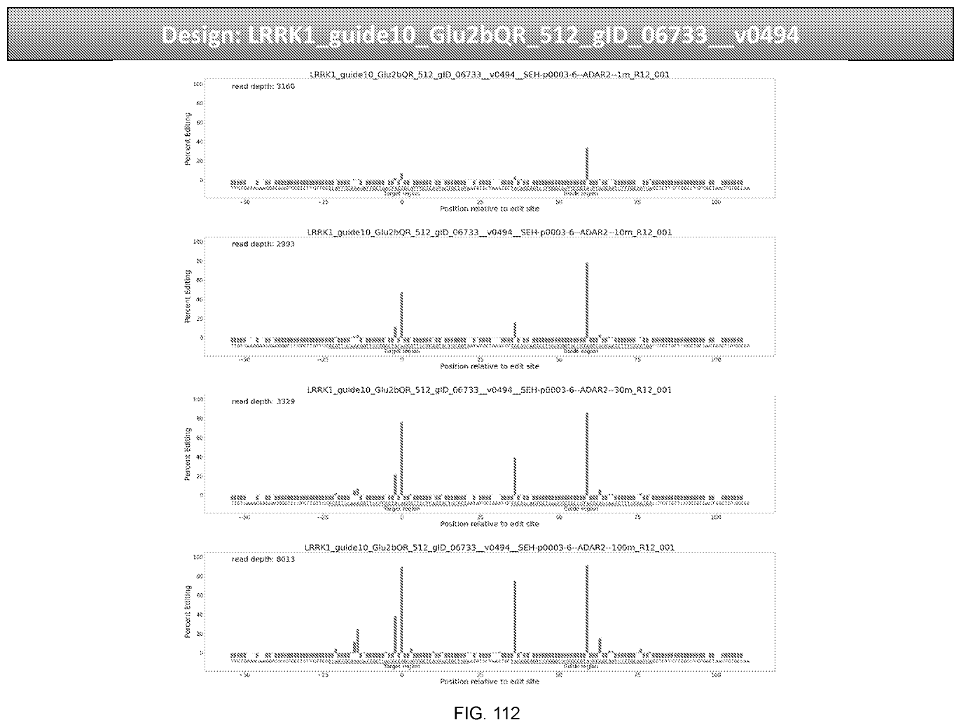

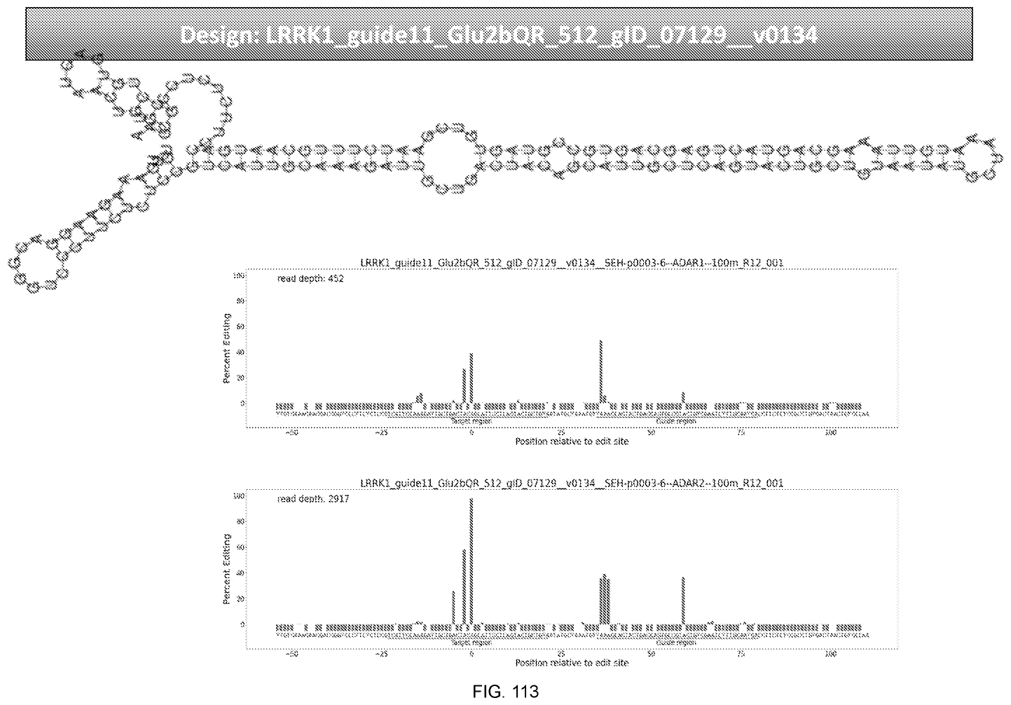

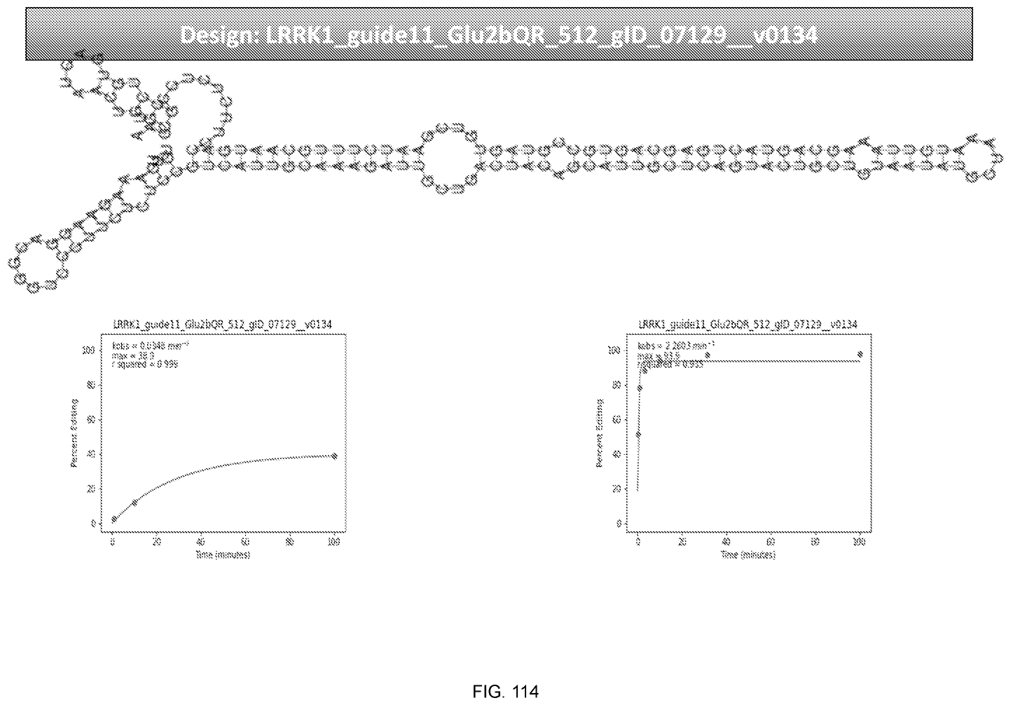

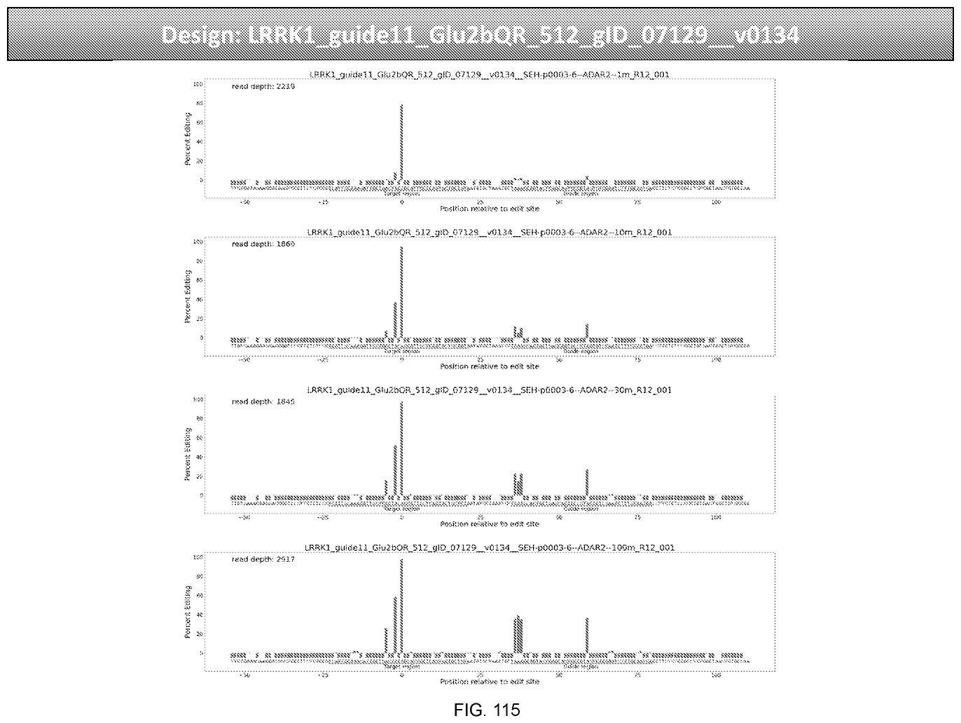

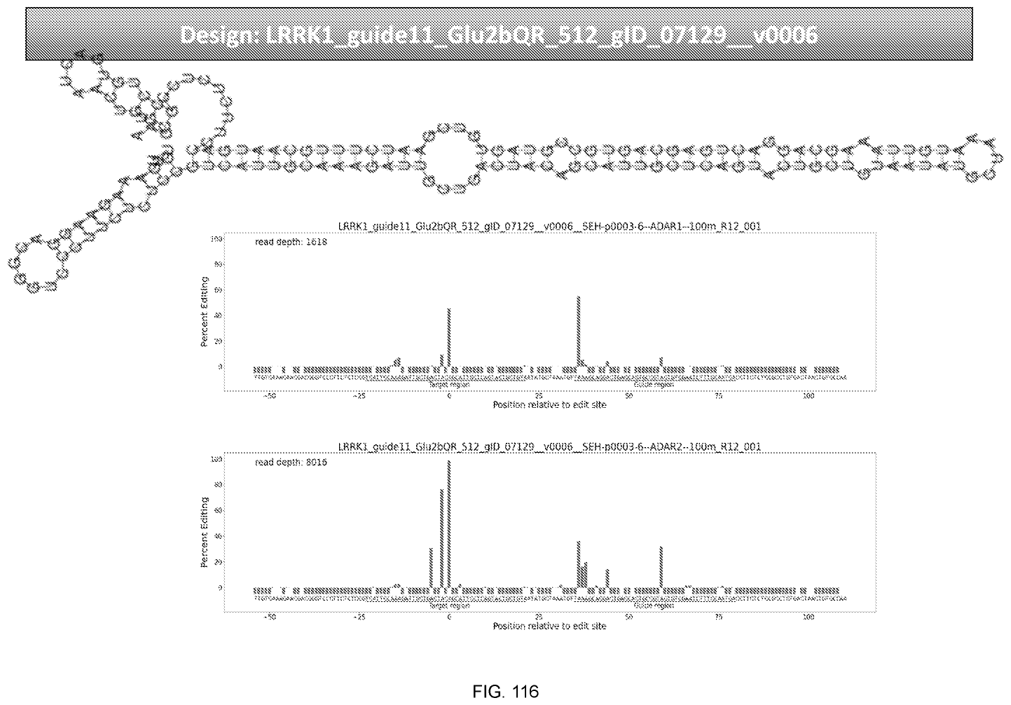

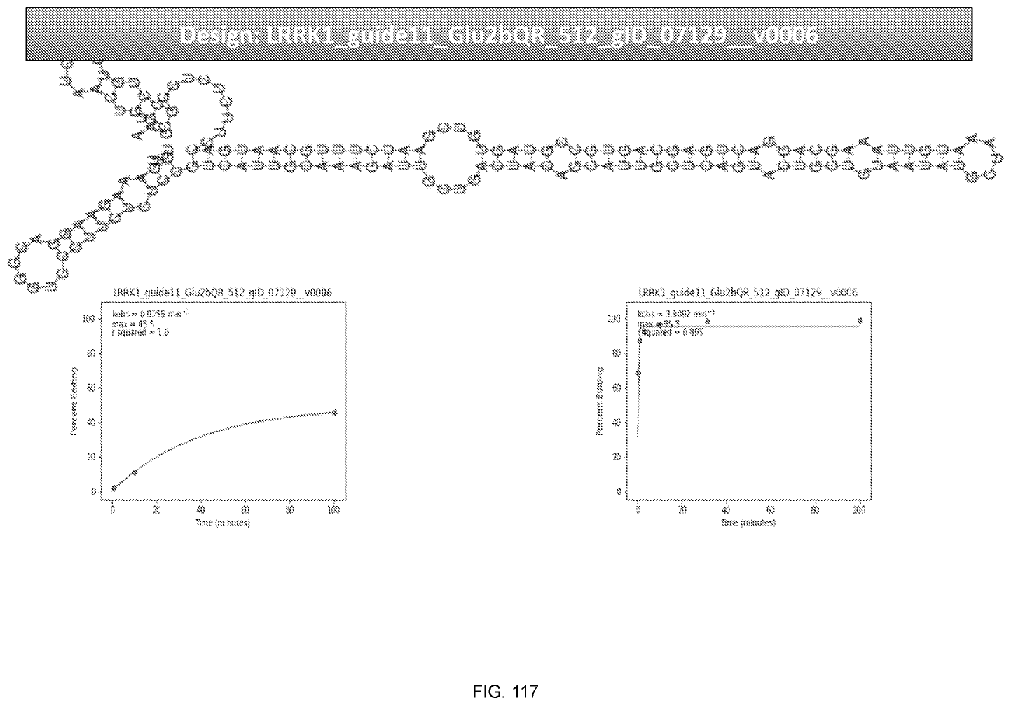

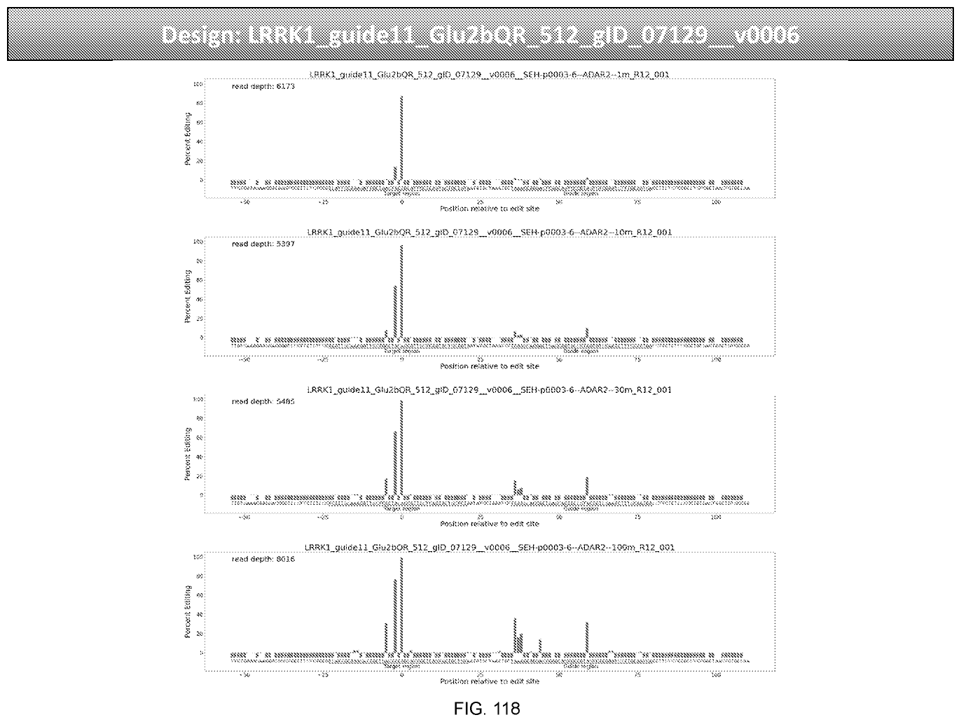

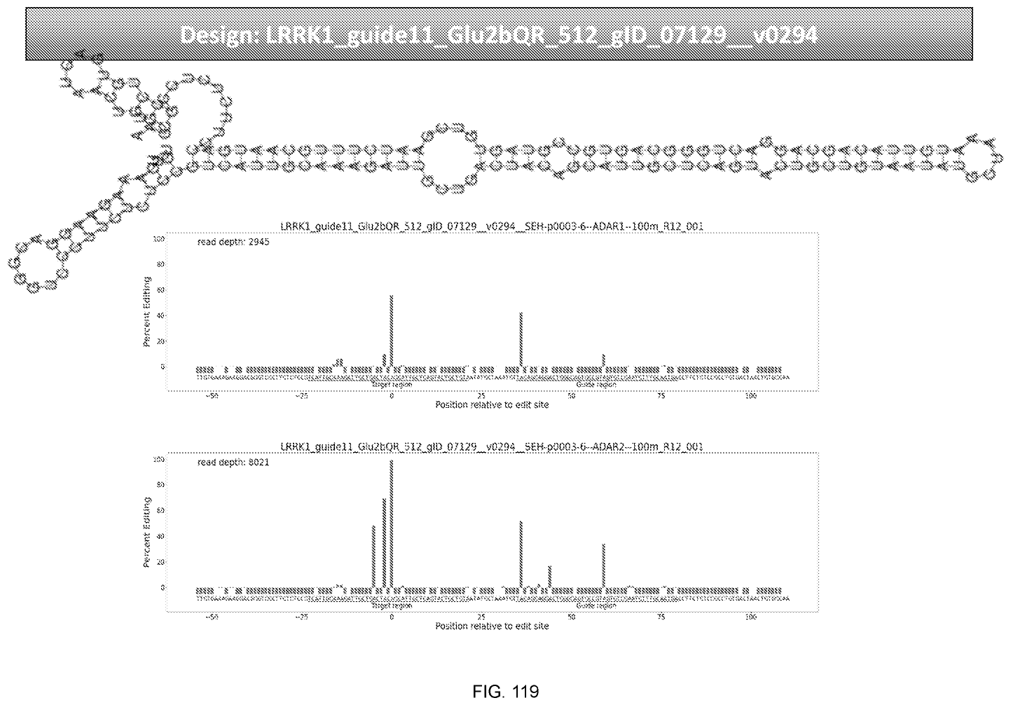

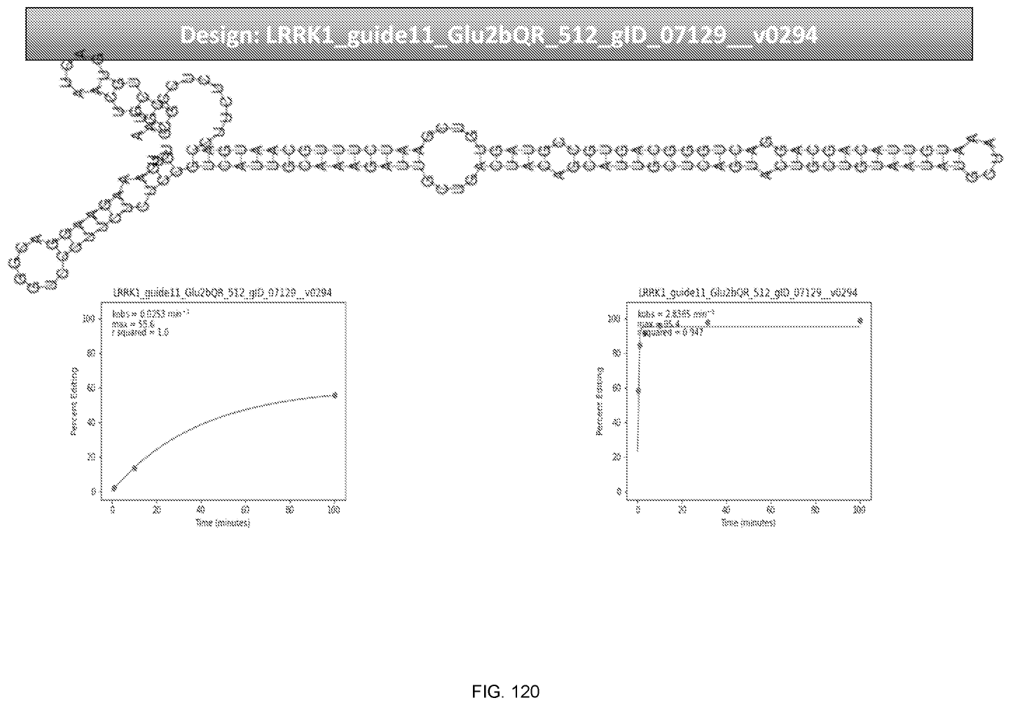

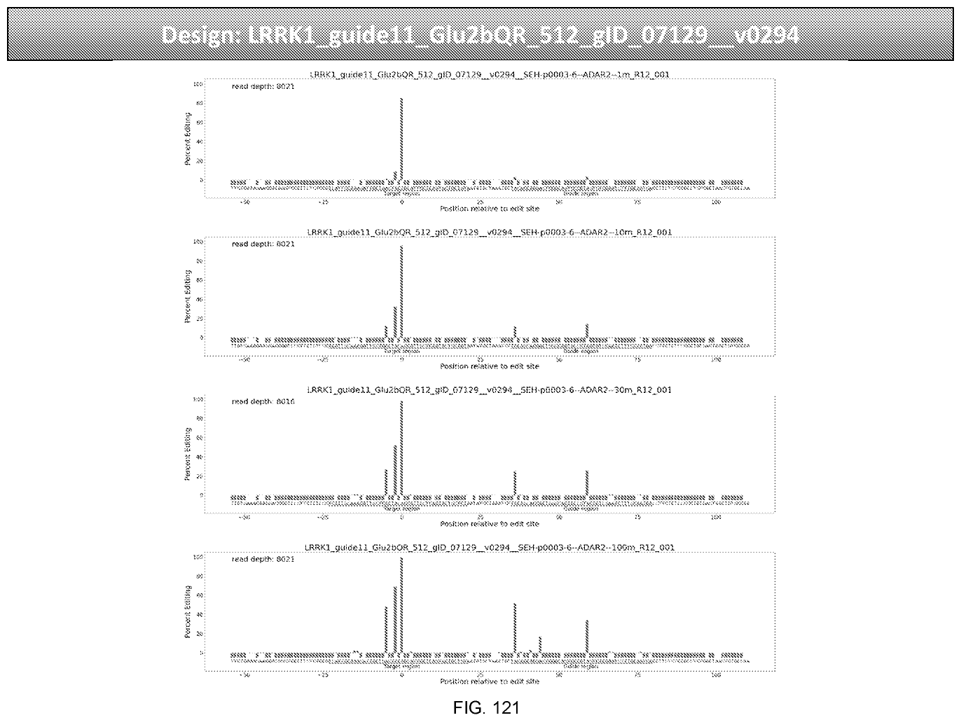

FIG. 20-FIG. 121 show exemplary engineered polynucleotide designs for targeting LRRK2 RNA, the percentage editing as a function of time for each engineered polynucleotide as determined by sequencing, and the editing at the target A to be edited (“0” on the x-axis) and at RNA editing at off-target positions (represented as black bars at positions that are not “0”).

Exemplary engineered polynucleotide sequences corresponding to FIG. 20-FIG. 121 are shown in Table 17.

TABLE 17
Exemplary Engineered Polynucleotide Sequences Targeting LRRK2 mRNA

| SEQ ID | | |
|---|---|---|
| NO: | ID | Sequence |
| 86 | LRRK1_guide10_ | TACAGCAGTACTGGGTGGTGCCGTAGTCAGCAATCTT |
| | Glu2bRG_512_gID_ | TGCAATGA |
| | 06733_v0446 | |
| 87 | LRRK1_guide11_ | TACAGCAGGACTGAGCAGTGCCGTAGTGTCGAATCTT |
| | Glu2bRG_512_gID_ | TGCAATGA |
| | 07219_v0262 | |
| 88 | LRRK1_guide10_ | TAAAGCAGGACTGAGTAGTGCCGTAGTCAGCAATCTT |
| | Glu2bQR_512_gID_ | TGCAATGA |
| | 06733_v0022 | |
| 89 | LRRK1_guide04_ | TACAGCAGTACTGAGCAGTGCCGTAGTGTCGTTTCTT |
| | Glu2bRG_128_gID_ | TGCAATGA |
| | 04357_v0094 | |
| 90 | LRRK1_guide04_ | TACAGCATTACTGAGCAGTGCCGTAGTGTCGTTTCTT |
| | Glu2bRG_128_gID_ | TGCAATGA |
| | 04357_v0126 | |
| 91 | LRRK1_guide11_ | TACAGCAGGACTGAGTAGTGCCGTAGTGTCGAATCTT |
| | Glu2bQR_512_gID_ | TGCAATGA |
| | 07129_v0278 | |
| 92 | LRRK1_guide10_ | TACAGCAGGACTGAGCGGTGCCGTAGTCAGCAATCTT |
| | Glu2bQR_512_gID_ | TGCAATGA |
| | 06733_v0270 | |
| 93 | LRRK1_guide10_ | TACAGCAGTACTGAGCGGTGCCGTAGTCAGCAATCTT |
| | Glu2bQR_512_gID_ | TGCAATGA |
| | 06733_v0398 | |
| 94 | LRRK1_guide10_ | TACAGCAGGACTGGGTGATGCCGTAGTCAGCAATCTT |
| | Glu2bQR_512_gID_ | TGCAATGA |
| | 06733_v0314 | |
| 95 | LRRK1_guide10_ | TAAAGCAGTACTGAGCGGTGCCGTAGTCAGCAATCTT |
| | Glu2bQR_512_gID_ | TGCAATGA |
| | 06733_v0142 | |
| 96 | LRRK1_guide10_ | TACAGCAGTCCTGGGTGGTGCCGTAGTCAGCAATCTT |
| | Glu2bQR_512_gID_ | TGCAATGA |
| | 06733_v0510 | |
| 97 | LRRK1_guide11_ | TACAGCAGGACTGGGTAGTGCCGTAGTGTCGAATCTT |
| | Glu2bQR_512_gID_ | TGCAATGA |
| | 07129_v0310 | |
| 98 | LRRKlguidelOGl | TACAGCAGGACTGAGCAGTGCCGTAGTCAGCAATCTT |
| | u2bQR_512_gID_06 | TGCAATGA |
| | 733_v0262 | |
| 99 | LRRK1_guide10_ | TAAAGCAGTACTGAGCAGTGCCGTAGTCAGCAATCTT |
| | Glu2bQR_512_gID_ | TGCAATGA |
| | 06733_vO134 | |
| 100 | LRRK1_guide11_ | TAAAGCAGGCCTGAGCAGTGCCGTAGTGTCGAATCTT |
| | Glu2bQR_512_gID_ | TGCAATGA |
| | 07129_v0070 | |
| 101 | LRRK1_guide11_ | TAAAGCAGGACTGGGCAGTGCCGTAGTGTCGAATCTT |
| | Glu2bQR_512_gID_ | TGCAATGA |
| | 07129_v0038 | |
| 102 | LRRK1_guide10_ | TACAGCAGGACTGGGCGATGCCGTAGTCAGCAATCTT |
| | Glu2bQR_512_gID_ | TGCAATGA |
| | 06733_v0298 | |
| 103 | LRRK1_guide10_ | TACAGCAGGACTGGGCAGTGCCGTAGTCAGCAATCTT |
| | Glu2bQR_512_gID_ | TGCAATGA |
| | 06733_v0294 | |
| 104 | LRRK1_guide10_ | TAAAGCAGGACTGGGCAGTGCCGTAGTCAGCAATCT |
| | Glu2bQR_512_gID_ | TTGCAATGA |
| | 06733_v0038 | |
| 105 | LRRK1_guide04_ | TACAGCATTACGGAGCAGTGCCGTAGTGTCGTTTCTT |
| | Glu2bRG_128_gID_ | TGCAATGA |
| | 04357_v0118 | |
| 106 | LRRK1_guide11_ | TACAGCAGGCCTGAGCAGTGCCGTAGTGTCGAATCTT |
| | Glu2bQR_512_gID_ | TGCAATGA |
| | 07129_v0326 | |
| 107 | LRRK1_guide11_ | TAAAGCAGGACTGGGTAGTGCCGTAGTGTCGAATCTT |
| | Glu2bQR_512_gID_ | TGCAATGA |
| | 07129_v0054 | |
| 108 | LRRK1_guide11_ | TACAGCAGTACTGAGCAGTGCCGTAGTGTCGAATCTT |
| | Glu2bQR_512_gID_ | TGCAATGA |
| | 07129_v0390 | |
| 109 | LRRK1_guide03_ | TACAGCAATACCGAGCAGTGCCGTAGTCAGCAATCTT |
| | Glu2bRG_128_gID_ | TGCAATGA |
| | 03961_v0014 | |
| 110 | LRRK1_guide10_ | TACAGCAGTACTGGGCGGTGCCGTAGTCAGCAATCTT |
| | Glu2bQR_512_gID_ | TGCAATGA |
| | 06733_v0430 | |
| 111 | LRRKl1_guide10_ | TACAGCAGGACTGGGTGGTGCCGTAGTCAGCAATCTT |
| | Glu2bQR_512_gID_ | TGCAATGA |
| | 06733_v0318 | |
| 112 | LRRK1_guide10_ | TAAAGCAGGACTGAGCAGTGCCGTAGTCAGCAATCT |
| | Glu2bQR_512_gID_ | TTGCAATGA |
| | 70633_v0006 | |
| 113 | LRRK1_guide11_ | TAAAGCAGGACTGAGTAGTGCCGTAGTGTCGAATCTT |
| | Glu2bQR_512_gID_ | TGCAATGA |
| | 07129_v0022 | |
| 114 | LRRK1_guide10_ | TACAGCAGTACTGAGTGGTGCCGTAGTCAGCAATCTT |
| | Glu2bQR_512_gID_ | TGCAATGA |
| | 06733_v0414 | |
| 115 | LRRK1_guide10_ | TACAGCAGGACTGGGCGGTGCCGTAGTCAGCAATCTT |
| | Glu2bQR_512_gID_ | TGCAATGA |
| | 06733_v0302 | |
| 116 | LRRK1_guide10_ | TACAGCAGTCCTGGGCGGTGCCGTAGTCAGCAATCTT |
| | Glu2bQR_512_gID_ | TGCAATGA |
| | 06733_v0494 | |
| 117 | LRRK1_guide11_ | TAAAGCAGTACTGAGCAGTGCCGTAGTGTCGAATCTT |
| | Glu2bQR_512_gID_ | TGCAATGA |
| | 10729_vO134 | |
| 118 | LRRK1_guide11_ | TAAAGCAGGACTGAGCAGTGCCGTAGTGTCGAATCTT |
| | Glu2bQR_512_gID_ | TGCAATGA |
| | 07129_v0006 | |
| 119 | LRRK1_guide11_ | TACAGCAGGACTGGGCAGTGCCGTAGTGTCGAATCTT |
| | Glu2bQR_512_gID_ | TGCAATGA |
| | 07129_v0294 | |

### Example 19: Engineered Polynucleotides Comprising Targeting LRRK2 mRNA

This example describes LRRK2 editing with engineered polynucleotides of the present disclosure. Self-annealing RNA structures comprising the engineered polynucleotide sequences of Table 18 and the sequences of the regions targeted by the engineered polynucleotides were contacted with an RNA editing entity (e.g., a recombinant ADAR1 and/or ADAR2) under conditions that allow for the editing of the regions targeted by the engineered polynucleotides. The regions targeted by the engineered polynucleotides were subsequently assessed for editing using next generation sequencing (NGS).

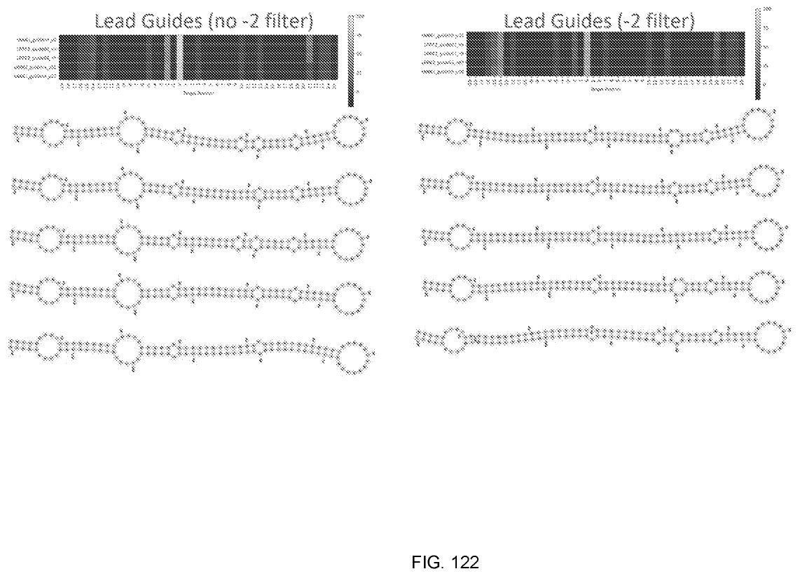

FIG. 122 shows heat maps and structures for exemplary engineered polynucleotide sequences targeting a LRRK2 mRNA. The heat map provides visualization of the editing profile at the 10 minute time point. 5 engineered polynucleotides for on-target editing (with no-2 filter) are in the left graph and 5 engineered polynucleotides for on-target editing with minimal-2 editing are depicted on the right graph. The corresponding predicted secondary structures are below the heat maps.

Exemplary engineered polynucleotide sequences corresponding to FIG. 122 are shown in Table 18.

TABLE 18
Exemplary Engineered Polynucleotide Sequences Targeting LRRK2 mRNA

| SEQ ID | | |
|---|---|---|
| NO: | ID | Sequence |
| 120 | LRRK2_guide04_v2 | GCTAAATCGATTCTAAAGCAGGACGGGGTGGTGCC |
| | | GTAGTGTCGTATCTTTGCACTTACAGTC |
| 121 | LRRK2_guide04_v3 | GCTAAATCGATTCTAAAGCAGGACTGGGTGGTGCC |
| | | GTAGTGTCGTATCTTTGCACTTACAGTC |
| 122 | LRRK2_guide04_v1 | GCTAAATCGATTCTAAAGCAGGACCGGGTGGTGCC |
| | | GTAGTGTCGTATCTTTGCACTTACAGTC |
| 123 | LRRK2_guide04_v51 | GCTAAATCGATTCTAGAGCAGGACTGGGTGGTGCC |
| | | GTAGTGTCGTATCTTTGCACTTACAGTC |
| 124 | LRRK2_guide04_v27 | GCTAAATCGATTCTACAGCAGGACTGGGTGGTGCC |
| | | GTAGTGTCGTATCTTTGCACTTACAGTC |
| 125 | LRRK2_guide03_v15 | GCTAAATCGATTCTAAAGCATGACTGGGTGGTGCC |
| | | GTAGTCAGCAATCTTTGCACTTACAGTC |
| 126 | LRRK2_guide03_v3 | GCTAAATCGATTCTAAAGCAGGACTGGGTGGTGCC |
| | | GTAGTCAGCAATCTTTGCACTTACAGTC |
| 127 | LRRK2_guide03_v9 | GCTAAATCGATTCTAAAGCAGTACTGGGTGGTGCC |
| | | GTAGTCAGCAATCTTTGCACTTACAGTC |
| 128 | LRRK2_guide03_v63 | GCTAAATCGATTCTAGAGCATGACTGGGTGGTGCC |
| | | GTAGTCAGCAATCTTTGCACTTACAGTC |
| 129 | LRRK2_guide03_v50 | GCTAAATCGATTCTAGAGCAGGACGGGGTGGTGCC |
| | | GTAGTCAGCAATCTTTGCACTTACAGTC |

### Example 20: Engineered Polynucleotides Comprising a Dumbbell Design and Targeting LRRK2 mRNA

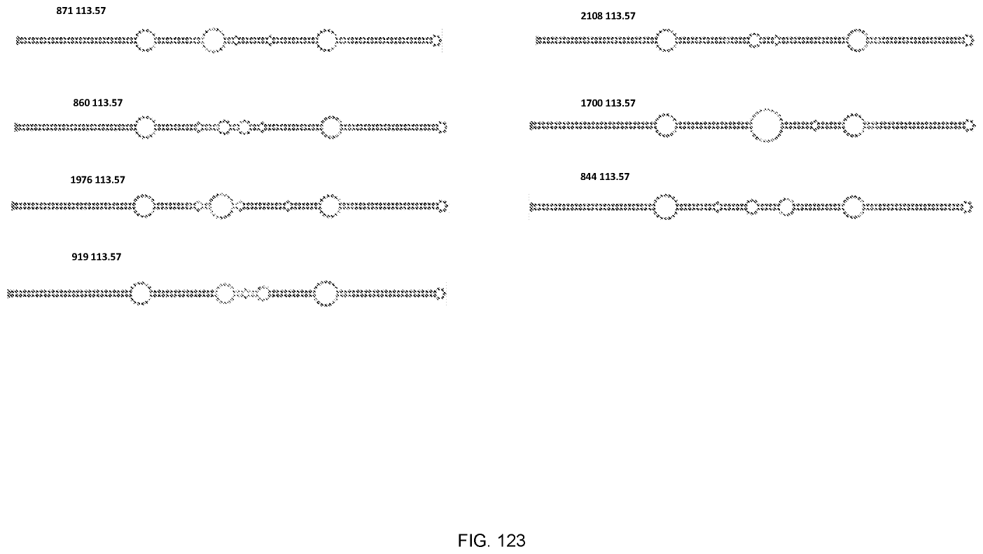

This example describes an engineered polynucleotide comprising a dumbbell design and targeting LRRK2 mRNA. A dumbbell design in an engineered polynucleotide comprises two symmetrical loops, wherein the target A to be edited is positioned between the two symmetrical loops for selective editing of the target A. The two symmetrical loops are each formed by 6 nucleotides on the engineered polynucleotide side of the dsRNA target and 6 nucleotides on the target RNA side of the dsRNA substrate. In this example, the target A is an A at position 6055 of a LRRK2 mRNA, which encodes for a pathogenic G2019S mutant protein. Exemplary engineered polynucleotides with a dumbbell design and targeting LRRK2 mRNA are shown in Table 19 and FIG. 123.

TABLE 19
Exemplary Dumbbell Engineered Polynucleotide Sequences

| SEQ ID NO: | ID | Sequence |
|---|---|---|
| 130 | 871 113.57 | CCCTGGTGTGCCCTCTGATGTTTTTTAGGGGATTCTACAGT |
| | | AGGACTGAGCACTGCCGAGCTGGGCAATCTTTGCATACTA |
| | | CGCAGCATTGGGATACAGTGTGAAAAGCAGCA |
| 131 | 860 113.57 | CCCTGGTGTGCCCTCTGATGTTTTTTAGGGGATTCTACAGC |
| | | AGTACAGAGGACTGCCGAGGTCACCAATCTTTGCATACTA |
| | | CGCAGCATTGGGATACAGTGTGAAAAGCAGCA |
| 132 | 1976 113.57 | CCCTGGTGTGCCCTCTGATGTTTTTTAGGGGATTCTACAAC |
| | | AGTACTGAGCTATCCCGAATTCAACAATCTTTGCATACTA |
| | | CGCAGCATTGGGATACAGTGTGAAAAGCAGCA |
| 133 | 919 113.57 | CCCTGGTGTGCCCTCTGATGTTTTTTAGGGGCTTCTACAGC |
| | | AGTTCGGAGGAATCCCGAGGTCAGCAATCTTTGCATACTA |
| | | CGCAGCATTGGGATACAGTGTGAAAAGCAGCA |
| 134 | 2108 113.57 | CCCTGGTGTGCCCTCTGATGTTTTTTAGGGGATTCTACGGC |
| | | GGTACTGACCAATCCCGTAGTTAGCAATCTTTGCATACTA |
| | | CGCAGCATTGGGATACAGTGTGAAAAGCAGC |
| 135 | 1700 113.57 | CCCTGGTGTGCCCTCTGATGTTTTTTAGGGGATTCTACCGC |
| | | AGTACTACCCGATCCCGTAGTCAGCAATCTTTGCATACTA |
| | | CGCAGCATTGGGATACAGTGTGAAAAGCAGCA |
| 136 | 844 113.57 | CCCTGGTGTGCCCTCTGATGTTTTTTAGGGGATTCTACGGC |
| | | AGTTCATAGCAATCCCGTAGTCAACAATCTTTGCCTACTAC |
| | | GCAGCATTGGGATACAGTGTGAAAAGCAGCA |

Self-annealing RNA structures comprising the engineered polynucleotide sequences of Table 19 and the sequences of the regions targeted by the engineered polynucleotides were contacted with an RNA editing entity (e.g., a recombinant ADAR1 and/or ADAR2) under conditions that allow for the editing of the regions targeted by the engineered polynucleotides. The regions targeted by the engineered polynucleotides were subsequently assessed for editing using next generation sequencing (NGS).

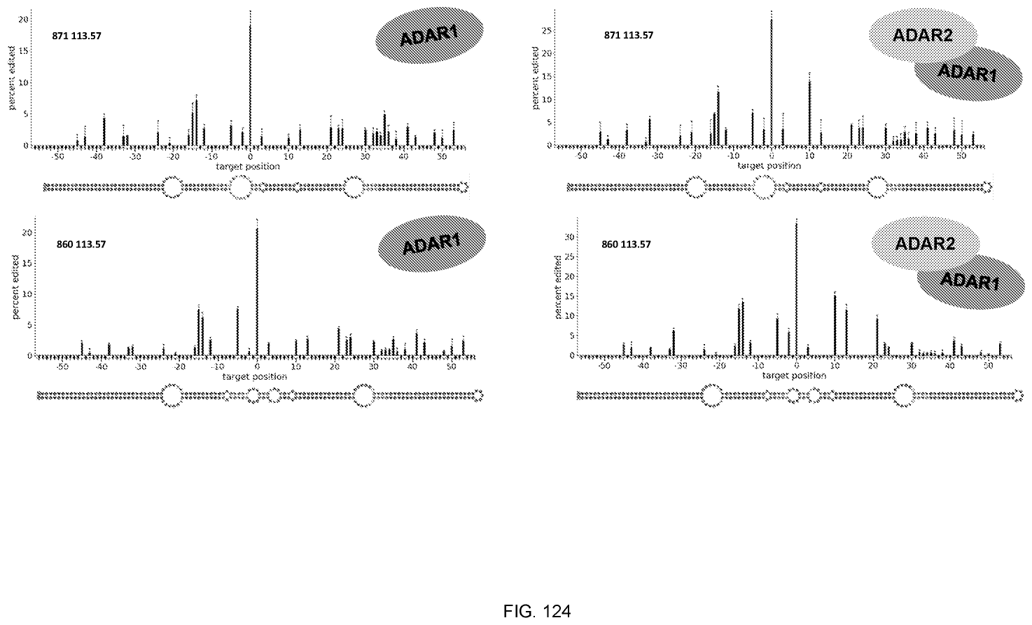

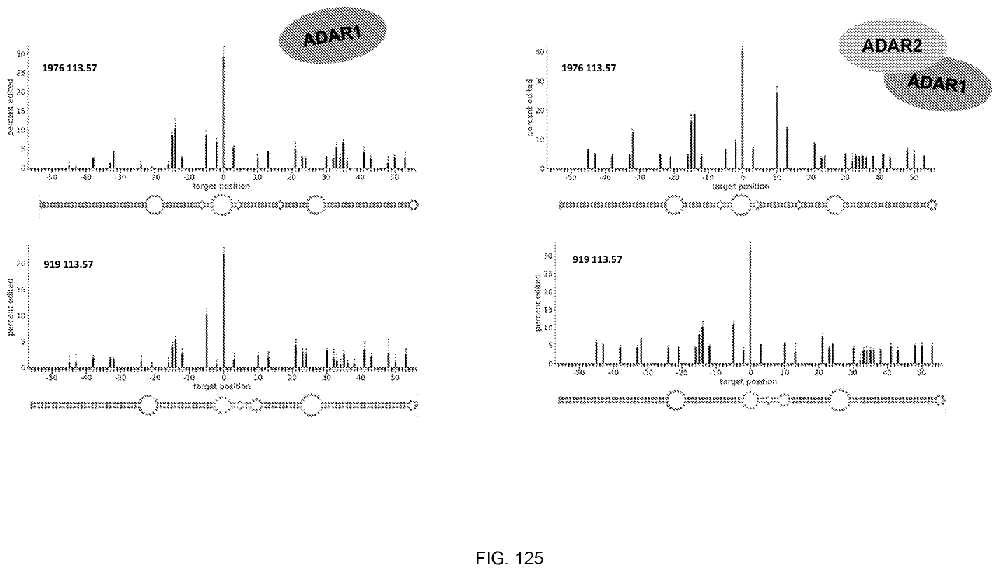

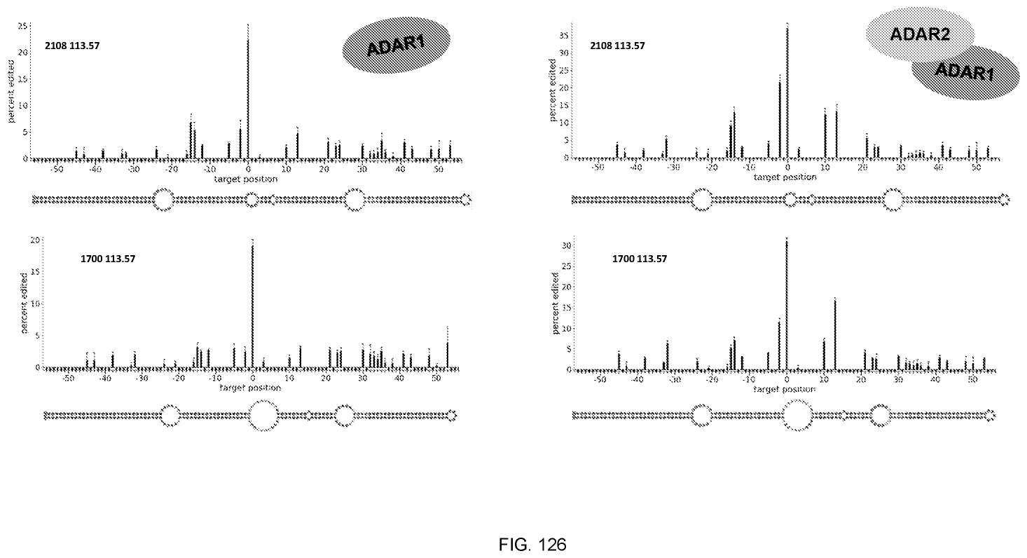

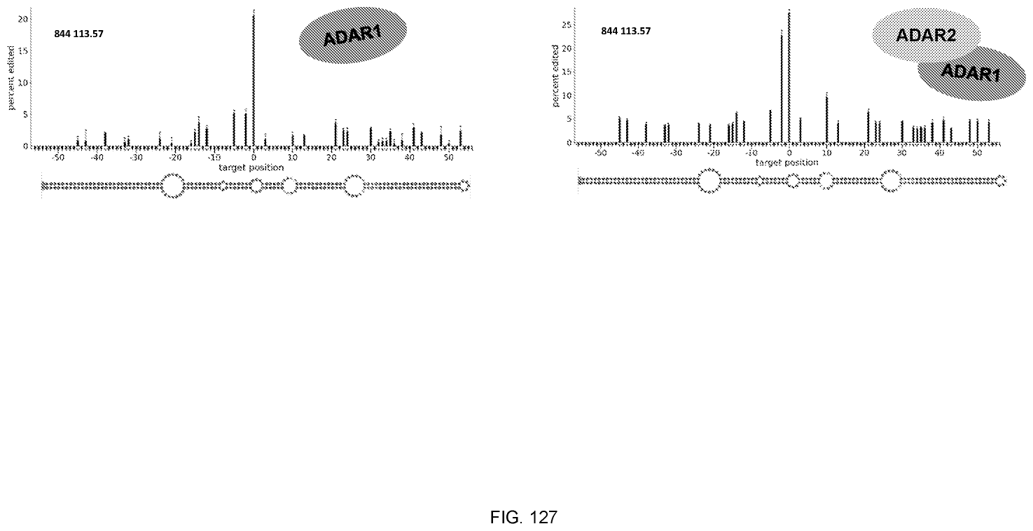

FIG. 124-FIG. 127 shows graphs of on-target and off-target ADAR1 editing and ADAR1+ADAR2 editing of the nucleotide of the codon encoding the G2019S mutation for exemplary dumbbell engineered polynucleotide sequences (see Table 19).

### Example 21: Engineered Polynucleotides Targeting LRRK2 mRNA

This example describes engineered polynucleotides that target LRRK2 mRNA. Self-annealing RNA structures comprising the engineered polynucleotide sequences of Table 20 and the sequences of the regions targeted by the engineered polynucleotides were contacted with an RNA editing entity (e.g., a recombinant ADAR1 and/or ADAR2) under conditions that allow for the editing of the regions targeted by the engineered polynucleotides. The regions targeted by the engineered polynucleotides were subsequently assessed for editing using next generation sequencing (NGS). The engineered polynucleotides of Table 20 showed specific editing of the A nucleotide at position 6055 of the mRNA encoding the LRRK2 G2019S.

TABLE 20
Exemplary engineered polynucleotides that target LRRK2 mRNA

| SEQ ID | | |
|---|---|---|
| NO: | ID | Sequence |
| 137 | LRRK2_bnPCR_ID | ATTCTACAGCACGACTGAGCAATGCCGTATTCAGCAATCT |
| | 3796_count1909 | TTGCA |
| 138 | LRRK2_bnPCR_ID | ATTCTAGAGCAGTACTGAGCAATGCCGTGGTTACCAATCT |
| | 47662_count362 | TTGCA |
| 139 | LRRK2_bnPCR_ID | ATTCTACAGCAGTAGTAAGCTTTGCCGAAGTGAGCAATCT |
| | 117434_count761 | TTACA |
| 140 | LRRK2_bnPCR_ID | ATTCTACAGCACGACTGAGCAAGGCCGTAGTCAGCGATCT |
| | 70875_count310 | TTGCA |
| 141 | LRRK2_bnPCR_ID | ATTCTACAGCACTACGGGGCAATGCCGAAGTCTGCAATCT |
| | 26374_count337 | TTGCA |
| 142 | LRRK2_bnPCR_ID | ATTCTACGGCGGTACTGACCAATCCCGTAGTTAGCAATCT |
| | 34601_count2108 | TTGCA |
| 143 | LRRK2_bnPCR_ID | ATTCTACAGCGGTACTCATCAATGCCGTAGTCGTCAATTTT |
| | 8689_count926 | TGCA |
| 144 | LRRK2_bnPCR_ID | ATTCTAAAGCCGTACTAAGCAGTACCGTAGTCCCCAATCT |
| | 4669_count1929 | TTGCA |
| 145 | LRRK2_bnPCR_ID | ATTCTAGAGCCATACTGTTCAATGCCGAAGTCAGCAATCT |
| | 24531_count2149 | TTGCA |
| 146 | LRRK2_bnPCR_ID | ATTCTACAGCGGTACTGGGCAATGCCGTGGTCTGCAATCT |
| | 38909_count2105 | TTGCA |
| 147 | LRRK2_bnPCR_ID | ATTCTACAGCTGTACTGAGGAATGCCGTCTTTACCAATCTT |
| | 13205_count2438 | TGCA |
| 148 | LRRK2_bnPCR_ID | ATTCTACAGCAGTACTTATCAGTGCCGTTGTTAGCAATCTT |
| | 74600_count480 | TGCA |
| 149 | LRRK2_bnPCR_ID | ATTCTATAGCTGGACTGAGCTCTGCCGTAGTCGTCAATCTT |
| | 11052_count1602 | TGCA |
| 150 | LRRK2_bnPCR_ID | ATTCTACAGCAATACTCAGCAGTGCCGTAGTCACCAATCT |
| | 22281_count2661 | TTGCA |
| 151 | LRRK2_bnPCR_ID | ATTCTACAACAGTACTGCGCACCGCCGTAGTCTGCAATCT |
| | 20990_count3129 | TTGCA |
| 152 | LRRK2_bnPCR_ID | ATTCTAGAGCCGTACTGCGCTTTGCCGTAGTCATCAATCTT |
| | 27527_count488 | TGCA |
| 153 | LRRK2_bnPCR_ID | ATTCTACAGCCGTACTGCCCAATGCCGAAGTAACCAATCT |
| | 18596_count3437 | TTGCA |
| 154 | LRRK2_bnPCR_ID | ATTCTACCGTGGGACTGAGCAGTCCCGTAGTCTGCAATCT |
| | 5048_count2719 | TTGCA |
| 155 | LRRK2_bnPCR_ID | ATTCTACAGCAGTACTGTTCAGTGCGCTAGTAAGCAATCT |
| | 3292_count1951 | TTGCA |
| 156 | LRRK2_bnPCR_ID | ATTCTACAGGTGTACTGTTCTATGCCGTTGTTAGCCATCTT |
| | 92487_count914 | TGCA |
| 157 | LRRK2_bnPCR_ID | ATTCTAGGGCAGTACTGAGAGATGCCGATGTCAGCAATCT |
| | 12934_count1814 | TTGCA |
| 158 | LRRK2_bnPCR_ID | ATTCTACAGCAGTATTGGGCAGTCCCGTAGTCAGCAATCT |
| | 22125_count837 | TTGCA |
| 159 | LRRK2_bnPCR_ID | ATTCTACAGCAGGACTGGGCGATCCCGTAGTCATCAATCT |
| | 31607_count1105 | TTGCA |
| 160 | LRRK2_bnPCR_ID | ATTCTACAGCAGTACTAAGCTATGGCGTACGCACCAATCT |
| | 17685_count2957 | TTGCA |
| 161 | LRRK2_bnPCR_ID | ATTCTACAGTAGGACTGAGCACTGCCGAGCTGGGCAATCT |
| | 29209_count871 | TTGCA |
| 162 | LRRK2_bnPCR_ID | ATTCTACCGCAGTACTGAGCAAAGCCGAGGTAAACAATCT |
| | 33976_count681 | TTGCA |
| 163 | LRRK2_bnPCR_ID | ATTCTACAGCAGTACTGAGCAATTCCGTTAACCGCAATCT |
| | 39201_count637 | TTGCA |
| 164 | LRRK2_bnPCR_ID | ATTCTACAGTAGTCCGGAGCTATTCCGTAGTCCGCAATCTT |
| | 87993_count219 | TGCA |
| 165 | LRRK2_bnPCR_ID | ATTCTACAGCAGGACTGAGCAATGGAGTAGTCGGCAATCT |
| | 31517_count1083 | TTGCA |
| 166 | LRRK2_bnPCR_ID | ATTCTACCGCAGTACTACCCGATCCCGTAGTCAGCAATCT |
| | 28134_count1700 | TTGCA |
| 167 | LRRK2_bnPCR_ID | ATTCTACAGGAGTTCTGAGCATTCCCGTCGTCATCAATCTT |
| | 38170_count773 | TGCA |
| 168 | LRRK2_bnPCR_ID | ATTCTACAGCAGTACAGAGGACTGCCGAGGTCACCAATCT |
| | 33405_count860 | TTGCA |
| 169 | LRRK2_bnPCR_ID | ATTCTACAGAAGGACCGAGCAGTCCCGTAGTCAGCAATCT |
| | 39919_count1633 | TTGCA |
| 170 | LRRK2_bnPCR_ID | ATTCTACAGCATTACTGAGCAATCCCGTATTAAGCAATCT |
| | 21333_count1914 | TTGCA |
| 171 | LRRK2_bnPCR_ID | ATTCTACAGCAGTACGAAGCAATCCCGCCGTTAGCAATCT |
| | 97672_count397 | TTGCA |
| 172 | LRRK2_bnPCR_ID | ATTCTACAGCAGTACTGTTCAATTCCGATGTAAGCAATCTT |
| | 36558_count1785 | TGCA |
| 173 | LRRK2_bnPCR_ID | ATTCTACAACAGTACTGAGCTATCCCGAATTCAACAATCT |
| | 16690_count1976 | TTGCA |
| 174 | LRRK2_bnPCR_ID | ATTCTACGGCAGTTCATAGCAATCCCGTAGTCAACAATCT |
| | 22357_count844 | TTGCC |
| 175 | LRRK2_bnPCR_ID | CTTCTACAGCAGTTCGGAGGAATCCCGAGGTCAGCAATCT |
| | 8030_count919 | TTGCA |
| 176 | LRRK2_bnPCR_ID | ATTCTACAGCTGTACTAGGCTATCCCGTAGTCCGCAATCTT |
| | 43647_count763 | TGCA |
| 177 | LRRK2_bnPCR_ID | ATTCTACAGCCGTACTGGGAAATCCCGTAGTCTGCAATCT |
| | 4389_count1135 | TTGCA |
| 178 | LRRK2_bnPCR_ID | ATTCTACAGAAGTACAGAGACATCCCGTAGTTAACAATCT |
| | 38794_count776 | TCGCA |
| 179 | LRRK2_bnPCR_ID | ATTCTACAGCCGTACTGAGCTATCCCGTAGTCCGCAATCTT |
| | 37802_count2433 | TGCA |
| 180 | LRRK2_bnPCR_ID | ATTCTACTGCAGCACCGAGCAATCCCGCATTCCCCAATCT |
| | 489_count3336 | TTGCA |
| 181 | LRRK2_bnPCR_ID | ATTCTACAGCAGTCCTGAGCAGTACCGTAGTCAGCAATCT |
| | 50412_count1318 | TTGCA |
| 182 | LRRK2_bnPCR_ID | ATTCTAAGGCAGTACTGAGCCGCGCCGTAGTCGGCAATCT |
| | 27560_count2218 | TTGCA |

### Example 22: Engineered Polynucleotides Targeting SNCA mRNA

This example describes engineered polynucleotides that target SNCA mRNA. Self-annealing RNA structures comprising the engineered polynucleotide sequences of Table 20 and the sequences of the regions targeted by the engineered polynucleotides were contacted with an RNA editing entity (e.g., a recombinant ADAR1 and/or ADAR2) under conditions that allow for the editing of the regions targeted by the engineered polynucleotides. The regions targeted by the engineered polynucleotides were subsequently assessed for editing using next generation sequencing (NGS). The engineered polynucleotides of Table 21 showed specific editing of the A nucleotide at translation initiation start site (TIS) of SNCA mRNA.

TABLE 21
Exemplary engineered polynucleotides that target SNCA mRNA

| SEQ ID | | |
|---|---|---|
| NO: | ID | Sequence |
| 183 | SNCA_guide01_v7 | GAAAGTACTTTGATGAATACATCCACGGCTAATGAATTCC |
| | | TTTAC |
| 184 | SNCA_guide02_v25 | GAAAGTCCTTTCAAGAATACATCCACGGCTATACTATTCC |
| | | TTTAC |
| 185 | SNCA_guide01_v24 | GAAAGTCCTTTGATGCATACATCCACGGCTAATGAATTCC |
| | | TTTAC |
| 186 | SNCA_guide02_v24 | GAAAGTCCTTTGATGCATACATCCACGGCTATACTATTCCT |
| | | TTAC |
| 187 | SNCA_guide02_v22 | GAAAGTCCTTTGAGGCATACATCCACGGCTATACTATTCC |
| | | TTTAC |
| 188 | SNCA_guide02_v21 | GAAAGTCCTTTGAGGAATACATCCACGGCTATACTATTCC |
| | | TTTAC |
| 189 | SNCA_guide02_v18 | GAAAGTCCTTTGAAGCATACATCCACGGCTATACTATTCC |
| | | TTTAC |
| 190 | SNCA_guide02_v11 | GAAAGTACTTTCACGAATACATCCACGGCTATACTATTCC |
| | | TTTAC |
| 191 | SNCA_guide02_v28 | GAAAGTACTTTCACGAATACATCCACGGCTATACTATTCC |
| | | TTTAC |
| 192 | SNCA_guide02_v26 | GAAAGTCCTTTCAAGCATACATCCACGGCTATACTATTCC |
| | | TTTAC |

What is claimed is:

1. An engineered polynucleotide comprising a targeting sequence that is at least partially complementary to a region of a target RNA, wherein the target RNA:
(a) encodes for a Leucine-rich repeat kinase 2 (LRRK2) polypeptide;
(b) comprises a non-coding sequence; or
(c) comprises (a) and (b),
wherein the engineered polynucleotide is configured upon binding to the region of the target RNA, in association with the target RNA, to form a structural feature which recruits an RNA editing entity, wherein the RNA editing entity, when associated with the engineered polynucleotide and the region of the target RNA, facilitates: an editing of a base of a nucleotide in the region of the target RNA, a modulation of translation of the LRRK2 polypeptide, or both.

2. The engineered polynucleotide of claim 1, wherein the targeting sequence is about: 40, 45, 60, 80, 100, 120, 200, or 300 nucleotides in length.

3. The engineered polynucleotide of claim 1 or 2, wherein the targeting sequence is about 100 nucleotides in length.

4. The engineered polynucleotide of any one of claims 1-3, wherein the targeting sequence that is at least partially complementary to the region of the target RNA comprises at least one nucleotide that is not complementary to a nucleotide in the region of the target RNA.

5. The engineered polynucleotide of claim 4, wherein the at least one nucleotide that is not complementary is an adenosine (A) in the region of the target RNA, and wherein the A is comprised in an A/C mismatch.

6. The engineered polynucleotide of claim 4, wherein the at least one nucleotide that is not complementary is an adenosine (A) in the region of the target RNA, and wherein the A is comprised in an internal loop or bulge.

7. The engineered polynucleotide of any one of claims 4-6, wherein the A is the base of the nucleotide in the region of the target RNA for editing.

8. The engineered polynucleotide of any one of claims 1-7, wherein the target RNA is selected from the group comprising: an mRNA, a pre-mRNA, a tRNA, a lncRNA, a lincRNA, a miRNA, a rRNA, a snRNA, a siRNA, a piRNA, a snoRNA, a exRNA, a scaRNA, a YRNA, an eRNA, and a hnRNA.

9. The engineered polynucleotide of any one of claims 1-8, wherein the target RNA is an mRNA.

10. The engineered polynucleotide of any one of claims 1-9, wherein the structural feature comprises: a bulge, a hairpin, an internal loop, and any combination thereof.

11. The engineered polynucleotide of any one of claims 1-10, wherein the structural feature comprises a bulge.

12. The engineered polynucleotide of claim 11, wherein the bulge is an asymmetric bulge.

13. The engineered polynucleotide of claim 11, wherein the bulge is a symmetric bulge.

14. The engineered polynucleotide of any one of claims 11-13, wherein the bulge is from 1-4 nucleotides in length.

15. The engineered polynucleotide of any one of claims 1-10, wherein the structural feature comprises a hairpin.

16. The engineered polynucleotide of any one of claims 1-10, wherein the structural feature comprises an internal loop.

17. The engineered polynucleotide of claim 16, wherein the internal loop is from 5-50 nucleotides in length.

18. The engineered polynucleotide of claim 16 or 17, wherein the internal loop is 6 nucleotides in length.

19. The engineered polynucleotide of any one of claims 1-18 comprising at least two internal loops.

20. The engineered polynucleotide of any one of claims 1-19 comprising two internal loops.

21. The engineered polynucleotide of claim 20, wherein the two internal loops are internal symmetrical loops.

22. The engineered polynucleotide of claim 20 or 21, wherein the two internal loops are internal symmetrical loops and each side of the two internal loop is 6 nucleotides in length.

23. The engineered polynucleotide of claim 16, wherein the internal loop is an asymmetrical internal loop.

24. The engineered polynucleotide of any one of claims 1-23 comprising a structured motif.

25. The engineered polynucleotide of claim 24, wherein the structured motif comprises at least two of: the bulge, the hairpin, and the internal loop.

26. The engineered polynucleotide of claim 25, wherein the structured motif comprises the bulge and the hairpin.

27. The engineered polynucleotide of claim 25, wherein the structured motif comprises the bulge and the internal loop.

28. The engineered polynucleotide of any one of claims 1-27, wherein the engineered polynucleotide lacks a recruiting domain.

29. The engineered polynucleotide of any one of claims 1-28, wherein the RNA editing entity comprises an adenosine deaminase acting on RNA (ADAR) polypeptide or biologically active fragment thereof or adenosine deaminases acting on tRNA (ADAT) polypeptide or biologically active fragment thereof.

30. The engineered polynucleotide of claim 29, wherein the ADAR polypeptide or biologically active fragment thereof comprises ADAR1 or ADAR2.

31. The engineered polynucleotide of any one of claims 1-30, wherein the engineered polynucleotide further comprises an RNA editing entity recruiting domain that is capable of recruiting the RNA editing entity.

32. The engineered polynucleotide of claim 31, wherein the RNA editing entity recruiting domain is at least 1 to about 75 nucleotides in length.

33. The engineered polynucleotide of claim 31 or 32, wherein the RNA editing entity recruiting domain is at least 30-50 nucleotides in length.

34. The engineered polynucleotide of any one of claims 31-33, wherein the RNA editing entity recruiting domain comprises a glutamate ionotropic receptor AMPA type subunit 2 (GluR2) sequence.

35. The engineered polynucleotide of claim 34, wherein the GluR2 sequence comprises at least about 80%, 85%, 90%, 95%, or 99% sequence identity to SEQ ID NO: 1.

36. The engineered polynucleotide of claim 34, wherein the GluR2 sequence comprises SEQ ID NO: 1.

37. The engineered polynucleotide of any one of claims 1-36, wherein the region is from 5 to 600 nucleotides in length of the target RNA, 40 to 400 nucleotides in length, or 80 to 120 nucleotides in length.

38. The engineered polynucleotide of any one of claims 1-37, wherein the region is from 50 to 200 nucleotides in length of the target RNA.

39. The engineered polynucleotide of any one of claims 1-38, wherein the region is about 100 nucleotides in length of the target RNA.

40. The engineered polynucleotide of any one of claims 1-39, wherein the region of the target RNA comprises at least 60%, 70%, 80%, 85%, 90%, 95%, 97%, 99%, or 100% sequence identity to SEQ ID NO: 73 or SEQ ID NO: 74.

41. The engineered polynucleotide of any one of claims 1-40, wherein the non-coding sequence comprises a three prime untranslated region (3′ UTR).

42. The engineered polynucleotide of any one of claims 1-41, wherein the non-coding sequence comprises a five prime untranslated region (5′ UTR).

43. The engineered polynucleotide of claim 42, wherein the editing of the base in the 5′UTR of the region of the target RNA results in at least partially regulating gene translation of the LRRK2 polypeptide.

44. The engineered polynucleotide of claim 42, wherein the editing of the base in the 5′UTR of the region of the target RNA results in facilitating regulation mRNA translation of: the LRRK2 polypeptide.

45. The engineered polynucleotide of any one of claims 1-44, wherein the target RNA encodes the LRRK2 polypeptide.

46. The engineered polynucleotide of claim 45, wherein the target RNA that encodes the LRRK2 polypeptide comprises at least a portion of: a poly(A) tail, a microRNA response element (MRE), AU-rich element (ARE), hnRNP binding sites or any combination thereof.

47. The engineered polynucleotide of claim 45 or 46, wherein the engineered polynucleotide is configured to modulate expression of the LRRK2 polypeptide.

48. The engineered polynucleotide of any one of claims 45-47, wherein the target RNA encodes a repeat domain of the LRRK2 polypeptide, a Ras-of-complex (Roc) GTPase domain of the LRRK2 polypeptide, a kinase domain of the LRRK2 polypeptide, a WD40 domain of the LRRK2 polypeptide, or a C-terminal of Roc (COR) domain of the LRRK2 polypeptide.

49. The engineered polynucleotide of claim 48, wherein the target RNA encodes the kinase domain of the LRRK2 polypeptide.

50. The engineered polynucleotide of any one of claims 1-49, wherein the region of the target RNA comprises a mutation as compared to an otherwise comparable region encoding a wildtype polypeptide.

51. The engineered polynucleotide of any one of claims 1-50, wherein the region of the target RNA comprises a mutation as compared to an otherwise comparable region encoding a wildtype LRRK2 polypeptide.

52. The engineered polynucleotide of claim 50 or 51, wherein the mutation comprises a polymorphism.

53. The engineered polynucleotide of any one of claims 50-52, wherein the mutation is a G to A mutation.

54. The engineered polypeptide of any one of claims 1-53, wherein the target RNA comprises at least 80%, 90%, 95%, 97%, 98%, 99%, or 100% sequence identity to any one of SEQ ID NO: 5-SEQ ID NO: 14.

55. The engineered polypeptide of any one of claims 1-54, wherein the target RNA encodes a LRRK2 polypeptide comprising at least 80%, 90%, 95%, 97%, 98%, 99%, or 100% sequence identity to any one of SEQ ID NO: 15-SEQ ID NO: 24.

56. The engineered polynucleotide of any one of claims 1-55, wherein the target RNA encodes a LRRK2 polypeptide comprising a mutation corresponding a G2019S of SEQ ID NO: 15.

57. The engineered polynucleotide of claim 1-56, wherein the editing of the base is editing of an A corresponding to the 6055th nucleotide in SEQ ID NO: 5.

58. The engineered polynucleotide of any one of claims 1-57, wherein the target RNA encodes a LRRK2 polypeptide comprising a mutation corresponding to a mutation of Table 3, or any combination of mutations of Table 3.

59. The engineered polynucleotide of any one of claims 1-58, wherein the engineered polynucleotide comprises at least 60%, 70%, 80%, 85%, 90%, 95%, 97%, 99%, or 100% sequence identity to any one of: SEQ ID NO: 66-SEQ ID NO: 72, SEQ ID NO: 81, SEQ ID NO: 82, or SEQ ID NO: 86-SEQ ID NO: 182.

60. The engineered polynucleotide of any one of claims 1-59, wherein when the engineered polynucleotide associates with the region of the target RNA, the association comprises hybridized polynucleotide strands.

61. The engineered polynucleotide of claim 60, wherein the hybridized polynucleotide strands at least in part form a double stranded RNA duplex.

62. The engineered polynucleotide of any one of claims 1-61, wherein the engineered polynucleotide further comprises a chemical modification.

63. The engineered polynucleotide of any one of claims 1-62, wherein the engineered polynucleotide comprises RNA, DNA, or both.

64. The engineered polynucleotide of claim 63, wherein the engineered polynucleotide comprises the RNA.

65. A vector that comprises the engineered polynucleotide of any one of claims 1-64.

66. The vector of claim 65, wherein the vector is a viral vector.

67. The vector of claim 66, wherein the viral vector is an AAV vector, and wherein the AAV vector is from an adeno-associated virus having a serotype selected from AAV1, AAV2, AAV3, AAV4, AAV5, AAV6, AAV7, AAV8, AAV9, AAV 10, AAV11, AAV 12, AAV13, AAV 14, AAV 15, AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68.

68. The vector of claim 67, wherein the AAV vector is a recombinant AAV (rAAV) vector, a hybrid AAV vector, a chimeric AAV vector, a self-complementary AAV (scAAV) vector, a single-stranded AAV or any combination thereof.

69. The vector of any one of claims 67-68, wherein the AAV vector comprises a genome comprising a replication gene and inverted terminal repeats from a first AAV serotype and a capsid protein from a second AAV serotype.

70. The vector of any one of claims 67-69, wherein the AAV vector is an AAV 2/5 vector, an AAV 2/6 vector, an AAV 2/7 vector, an AAV2/8 vector, or an AAV 2/9 vector.

71. The vector of any one of claims 67-70, wherein the inverted terminal repeats comprise a 5′ inverted terminal repeat, a 3′ inverted terminal repeat, and a mutated inverted terminal repeat.

72. The vector of claim 71, wherein the mutated inverted terminal repeat lacks a terminal resolution site.

73. A pharmaceutical composition in unit dose form that comprises: (a) the engineered polynucleotide of any one of claims 1-64; the vector of any one of claims 65-72, or any combination thereof; and (b) a pharmaceutically acceptable excipient, diluent, or carrier.

74. A method of making a pharmaceutical composition comprising admixing the engineered polynucleotide of any one of claim 1-64 with a pharmaceutically acceptable excipient, diluent, or carrier.

75. An isolated cell comprising the engineered polynucleotide of any one of claims 1-64, the vector of any one of claims 65-74, or both.

76. A kit comprising the engineered polynucleotide of any one of claims 1-64, the vector of any one of claims 65-74, or both in a container.

77. A method of making a kit comprising inserting the engineered polynucleotide of any one of claims 1-64, the vector of any one of claims 65-74, or both in a container.

78. A method of treating or preventing a disease or condition in a subject in need thereof, the method comprising administering to a subject in need thereof: (a) the vector of any one of claims 65-74; (b) the pharmaceutical composition of claim 73; or (c) (a) and (b).

79. The method of claim 78, wherein the administering comprises administering a therapeutically effective amount of the vector.

80. The method of claim 78 or 79, wherein the administering at least partially treats or prevents at least one symptom of the disease or the condition in the subject in need thereof.

81. The method of any one of claims 78-80, wherein the vector further comprises or encodes a second engineered polynucleotide.

82. The method of any one of claims 78-81, further comprising administering a second vector that comprises or encodes a second engineered polynucleotide.

83. The method of claim 81 or 82, wherein the second engineered polynucleotide comprises a second targeting sequence that at least partially hybridizes to a region of a second target RNA.

84. The method of claim 83, wherein the second targeting sequence of the second engineered polynucleotide is at least partially complementary to the region of the second target RNA.

85. The method of any one of claims of claim 83 or 84, wherein the second target RNA encodes for a polypeptide that comprises: alpha-synuclein (SNCA), glucosylceramidase beta (GBA), PTEN-induced kinase 1 (PINK1), Tau, biologically active fragment of any of these, or any combination thereof.

86. The method of claim 85, wherein the second target RNA encodes for the SNCA polypeptide or biologically active fragment thereof.

87. The method of any one of claims 81-86, wherein the second engineered polynucleotide is configured to facilitate an editing of a base of a nucleotide of a polynucleotide of a region of the second target RNA by the RNA editing entity.

88. The method of claim 87, wherein the editing results in reduced expression of a polypeptide encoded by the second target RNA.

89. The method of any one of claims 81-88, wherein the second engineered polynucleotide comprises at least 80%, 90%, 95%, 97%, 98%, 99%, or 100% sequence identity to any one of SEQ ID NO: 25-SEQ ID NO: 33.

90. The method of any one of claims 81-89, wherein the second engineered polynucleotide encodes a SCNA polypeptide comprising at least 80%, 90%, 95%, 97%, 98%, 99%, or 100% sequence identity to any one of SEQ ID NO: 34-SEQ ID NO: 36.

91. The method of any one of claims 81-90, wherein the second engineered polynucleotide encodes a SNCA polypeptide comprising a mutation corresponding to a mutation of Table 6, or any combination of mutations of Table 6.

92. The method of any one of claims 81-91, wherein the second engineered polynucleotide facilitates editing of an Adenosine (A) of a translational initiation site of the second target RNA that encodes a SNCA polypeptide.

93. The method of any one of claims 81-88, wherein the second engineered polynucleotide comprises at least 80%, 90%, 95%, 97%, 98%, 99%, or 100% sequence identity to any one of SEQ ID NO: 37-SEQ ID NO: 48.

94. The method of any one of claim 81-88 or 93, wherein the second engineered polynucleotide facilitates editing of an Adenosine (A) of a translational initiation site of the second target RNA that encodes a Tau polypeptide.

95. The method of any one of claims 81-88, wherein the second engineered polynucleotide comprises at least 80%, 90%, 95%, 97%, 98%, 99%, or 100% sequence identity to SEQ ID NO: 49.

96. The method of any one of claim 81-88 or 95, wherein the second engineered polynucleotide facilitates editing of an Adenosine (A) of a translational initiation site of the second target RNA that encodes a PINK1 polypeptide.

97. The method of any one of claims 81-88, wherein the second engineered polynucleotide comprises at least 80%, 90%, 95%, 97%, 98%, 99%, or 100% sequence identity to any one of SEQ ID NO: 50-SEQ ID NO: 54.

98. The method of any one of claim 81-88 or 97, wherein the second engineered polynucleotide facilitates editing of an Adenosine (A) of a translational initiation site of the second target RNA that encodes a GBA polypeptide.

99. The method of any one of claims 81-92, wherein the second engineered polynucleotide comprises at least 60%, 70%, 80%, 85%, 90%, 95%, 97%, 99%, or 100% sequence identity to any one of: SEQ ID NO: 183-SEQ ID NO: 192.

100. The method of any one of claims 78-99, wherein the disease or condition is of a central nervous system (CNS), gastrointestinal (GI) tract, or both.

101. The method of claim 100, wherein the disease is of both, and wherein the disease is Parkinson's Disease.

102. The method of claim 100, wherein the disease is of the GI tract, and wherein the disease is Crohn's disease.

103. The method of any one of claims 78-102, further comprising administering a secondary therapy.

104. The method of claim 103, wherein the secondary therapy is administered concurrent or sequential to the vector.

105. The method of claims 103-104, wherein the secondary therapy comprises at least one of a probiotic, a carbidopa, a levodopa, a MAO B inhibitor, a catechol O-methyltransferase (COMT) inhibitor, a anticholinergic, a amantadine, a deep brain stimulation, a salt of any of these, or any combination thereof.

106. The method of any one of claims 103-105, wherein the administering of the vector, the secondary therapy, or both are independently performed at least about: 1 time per day, 2 times per day, 3 times per day, 4 times per day, once a week, twice a week, 3 times a week, biweekly, bimonthly, monthly, or yearly.

107. The method of any one of claims 78-106, further comprising monitoring the disease or condition of the subject.

108. The method of any one of claims 78-107, wherein the vector is comprised in a pharmaceutical composition in unit dose form.

109. The method of any one of claims 78-108, wherein the subject is diagnosed with the disease or the condition prior to the administering.

110. The method of claim 109, wherein the diagnosing is via an in vitro assay.

111. The method of any one of claims 78-110, wherein the editing of the base of the nucleotide of the polynucleotide of the region of the target RNA comprises at least about 3%, 5%, 10%, 15%, or 20% editing as measured by sequencing.

112. The method of claim 111, wherein the second target RNA encodes for the SNCA polypeptide, and wherein the editing of the base of the nucleotide of the polynucleotide of the region of the target RNA by an ADAR polypeptide results in a modified polypeptide that comprises a change in a residue, as compared to an unmodified polypeptide encoded by the target RNA, that comprises:
(a) an adenine to an inosine at a position corresponding to position 2019 of the LRRK2 polypeptide of SEQ ID NO: 15;
(b) an adenine to an inosine at a position corresponding to position 30 or 53 of the SNCA polypeptide of SEQ ID NO: 34; or
(c) (a) and (b).


SEQUENCE LISTING (284 sequences)

SEQ ID NO: 1 - RNA, 45 nt, Unknown
  source: /note=“Description of Unknown: GluR2 sequence”
guggaauaguauaacaauaugcuaaauguuguuauaguaucccac

SEQ ID NO: 2-4 - skipped (3 entries)

SEQ ID NO: 5 - DNA, 9239 bp, Homo sapiens
ggggcccgcggggagcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaaaactttaaa

SEQ ID NO: 6 - DNA, 9319 bp, Homo sapiens
cctgagtgggggaggaggaagccgagcaggagggctccggagagggagggcaacgcggggcggggagctgcctccttcctcataaacaggcgggcgtgggcgccgatggggcccgcggggagcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaa

SEQ ID NO: 7 - DNA, 18530 bp, Homo sapiens
cctgagtgggggaggaggaagccgagcaggagggctccggagagggagggcaacgcggggcggggagctgcctccttcctcataaacaggcgggcgtgggcgccgatggggcccgcggggagcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaacctgagtgggggaggaggaagccgagcaggagggctccggagagggagggcaacgcggggcggggagctgcctccttcctcataaacaggcgggcgtgggcgccgatggggcccgcggggagcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaa

SEQ ID NO: 8 - DNA, 7989 bp, Homo sapiens
ttcaaacatcataagaccggcactctctcccaaagatacaagctgtagcaaggagttttgtgcatatcagtttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaaaactt

SEQ ID NO: 9 - DNA, 5180 bp, Homo sapiens
cctgagtgggggaggaggaagccgagcaggagggctccggagagggagggcaacgcggggcggggagctgcctccttcctcataaacaggcgggcgtgggcgccgatggggcccgcggggagcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagtttgtctgaccacaggcctgtgatagagcttccccattgtgagaactctgaaattatcatccgactatatgaaatgccttattttccaatgggattttggtcaagattaat

SEQ ID NO: 10 - DNA, 4868 bp, Homo sapiens
cctgagtgggggaggaggaagccgagcaggagggctccggagagggagggcaacgcggggcggggagctgcctccttcctcataaacaggcgggcgtgggcgccgatggggcccgcggggagcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaaggagtccttcttcattttcaagacccagcactgcagttaagtgacttgtactttgtggaacccaagtggctttgtaaaatcatggcacagg

SEQ ID NO: 11 - DNA, 6165 bp, Homo sapiens
gaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaaaactt

SEQ ID NO: 12 - DNA, 5410 bp, Homo sapiens
gagtagagaaactgcatctttctcacaataaactgaaagaggagcagaatgaagtcacagataatggataaccaaatccattttgtgattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggcattatttcgcgtagagatgtggaaaaatttctttcaaaaaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaagagagaaataggaattgtctttggataggaaaattattctctcctcttgtaaatatttattttaaaaatgttcacatggaaagggtactcacattttttgaaatagctcgtgtgtatgaaggaatgttattatttttaatttaaatatatgtaaaaatacttaccagtaaatgtgtattttaaagaactatttaaaacacaatgttatatttcttataaataccagttactttcgttcattaattaatgaaaataaatctgtgaagtacctaatttaagtactcatactaaaatttataaggccgataattttttgttttcttgtctgtaatggaggtaaactttattttaaattctgtgcttaagacaggactattgcttgtcgatttttctagaaatctgcacggtataatgaaaatattaagacagtttcccatgtaatgtattccttcttagattgcatcgaaatgcactatcatatatgcttgtaaatattcaaatgaatttgcactaataaagtcctttgttggtatgtgaattctctttgttgctgttgcaaacagtgcatcttacacaacttcactcaattcaaaagaaaactccattaaaagtactaatgaaaaaacatgacatactgtcaaagtcctcatatctaggaaagacacagaaactctctttgtcacagaaactctctgtgtctttcctagacataatagagttgtttttcaactctatgtttgaatgtggataccctgaattttgtataattagtgtaaatacagtgttcagtccttcaagtgatatttttatttttttattcataccactagctacttgttttctaatctgcttcattctaatgcttatattcatcttttccctaaatttgtgatgctgcagatcctacatcattcagatagaaacctttttttttttcagaattatagaattccacagctcctaccaagaccatgaggataaatatctaacacttttcagttgctgaaggagaaaggagctttagttatgatggataaaaatatctgccaccctaggcttccaaattatacttaaattgtttacatagcttaccacaataggagtatcagggccaaatacctatgtaataatttgaggtcatttctgctttaggaaaagtactttcggtaaattctttggccctgaccagtattcattatttcagataattccctgtgataggacaactagtacatttaatattctcagaacttatggcattttactatgtgaaaactttaaatttatttatattaagggtaatcaaattcttaaagatgaaagattttctgtattttaaaggaagctatgctttaacttgttatgtaattaacaaaaaaatcatatataatagagctctttgttccagtgttatctctttcattgttactttgtatttgcaattttttttaccaaagacaaattaaaaaaatgaataccatatttaaatggaataataaaggttttttaaaaactt

SEQ ID NO: 13 - DNA, 4153 bp, Homo sapiens
cctgagtgggggaggaggaagccgagcaggagggctccggagagggagggcaacgcggggcggggagctgcctccttcctcataaacaggcgggcgtgggcgccgatggggcccgcggggagcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgc

SEQ ID NO: 14 - DNA, 4132 bp, Homo sapiens
cctgagtgggggaggaggaagccgagcaggagggctccggagagggagggcaacgcggggcggggagctgcctccttcctcataaacaggcgggcgtgggcgccgatggggcccgcggggagcgctggctgcgggcggtgagctgagctcgcccccggggagctgtggccggcgcccctgccggttccctgagcagcggacgttcatgctgggagggcggcgggttggaagcaggtgccaccatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcgcgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatattggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagaggagcagaatgaagtcacagataatggataaccaaatccattttgtgattcctcctgagattggctgtcttgaaaatctgacatctctggatgtcagttacaacttggaactaa

SEQ ID NO: 15 - protein, 2527 aa, Homo sapiens
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 16 - protein, 2521 aa, Homo sapiens
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEEIQSCLTVWDINLPHEVQNLEKHIEVRKELAEKMRRTSVE

SEQ ID NO: 17 - protein, 2503 aa, Homo sapiens
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLEIQSCLTVWDINLPHEVQNLEKHIEVRKELAEKMRRTSVE

SEQ ID NO: 18 - protein, 2126 aa, Homo sapiens
MLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 19 - protein, 1611 aa, Homo sapiens
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQFV

SEQ ID NO: 20 - protein, 1524 aa, Homo sapiens
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKESFFIFKTQHCS

SEQ ID NO: 21 - protein, 1499 aa, Homo sapiens
MIHSTLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 22 - protein, 1281 aa, Homo sapiens
MKSQIMDNQIHFVIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 23 - protein, 1271 aa, Homo sapiens
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEVSSTAIKKGCAL

SEQ ID NO: 24 - protein, 1268 aa, Homo sapiens
MASGSCQGCEEDEETLKKLIVRLNNVQEGKQIETLVQILEDLLVFTYSERASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEEQNEVTDNG

SEQ ID NO: 25 - DNA, 3177 bp, Homo sapiens
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 26 - DNA, 3396 bp, Homo sapiens
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 27 - DNA, 3030 bp, Homo sapiens
gcttctccattctggtgtgatccaggaacagctgtcttccagctctgaaagagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 28 - DNA, 3312 bp, Homo sapiens
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 29 - DNA, 3099 bp, Homo sapiens
gcttctccattctggtgtgatccaggaacagctgtcttccagctctgaaagagggctgagagattaggctgcttctccgggatccgcttttccccgggaaacgcgaggatgctccatggagctgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 30 - DNA, 3041 bp, Homo sapiens
ggcgacgaccagaaggggcccaagagagggggcgagcgaccgagcgccgcgacgcggaagtgagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 31 - DNA, 3465 bp, Homo sapiens
ggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcagggctgagagattaggctgcttctccgggatccgcttttccccgggaaacgcgaggatgctccatggagctgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 32 - DNA, 3091 bp, Homo sapiens
gagtgtgagcggcgcctgctcagggtagatagctgagggcgggggtggatgttggatggattagaaccatcacacttgggcctgctgtttgcctgagtttgaaccacaccccgatgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 33 - DNA, 2984 bp, Homo sapiens
gctctaattctctgcaccttctcaagcattgtgcagattggttttctggattatcagcctgaaggacaaaacgaagaaacagccattagctcctgtctcccattgtctgagagctgccactaggatattaacttcctgaaattctgcagaaatctcctcttactttggcactggagatgcccatacgcagaaagcaaaaaggcacagcatatttaaggaagctcataagaaacagtgcatccagaagtggcgagaattggaggaatggacatgagactctaagaaccagcgcctttgatgttccttttgatctgttatgtagctcttcttgtacacagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 34 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 35 - protein, 112 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKEGYQDYEPEA

SEQ ID NO: 36 - protein, 92 aa, Homo sapiens
MPIRRKQKGTAYLRKLIRNSASRSGENWRNGHETLRTSAFDVPFDLLCSSSCTQNEEGAPQEGILEDMPVDPDNEAYEMPSEEGYQDYEPEA

SEQ ID NO: 37 - DNA, 6590 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 38 - DNA, 5639 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 39 - DNA, 5465 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 40 - DNA, 5372 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 41 - DNA, 5552 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 42 - DNA, 6644 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctgcgactaagcaagtccagagaagaccaccccctgcagggcccagatctgagagaggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 43 - DNA, 5459 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 44 - DNA, 5546 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 45 - DNA, 6815 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaacaccccactcctggtagctcagaccctctgatccaaccctccagccctgctgtgtgcccagagccaccttcctctcctaaatacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 46 - DNA, 6524 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagaggagttgagagttccgggccggcagaggaaggcgcctgaaaggcccctggccaatgagattagcgcccacgtccagcctggaccctgcggagaggcctctggggtctctgggccgtgcctcggggagaaagagccagaagctcccgtcccgctgaccgcgagccttcctcagcaccgtcccgtttgcccagcgcctcctccaacaggaggccctcaggagccctccctggagtggggacaaaaaggcggggactgggccgagaagggtccggcctttccgaagcccgccaccactgcgtatctccacacagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 47 - DNA, 4139 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 48 - DNA, 5525 bp, Homo sapiens
gcagtcaccgccacccaccagctccggcaccaacagcagcgccgctgccaccgcccaccttctgccgccgccaccacagccaccttctcctcctccgctgtcctctcccgtcctcgcctctgtcgactatcagacaggctctgctgatgctgtccctctcctgttcagtcgtgccctcaccgttaaagagaaagagcaaactgctgggcagcagcattgatttttttaatgaagtggaaagagagctgggaataacaagtcgggcccacctcacctgcctcacctggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 49 - DNA, 2657 bp, Homo sapiens
agaggcaccgccccaagtttgttgtgaccggcgggggacgccggtggtggcggcagcggcggctgcgggggcaccgggccgcggcgccaccatggcggtgcgacaggcgctgggccgcggcctgcagctgggtcgagcgctgctgctgcgcttcacgggcaagcccggccgggcctacggcttggggcggccgggcccggcggcgggctgtgtccgcggggagcgtccaggctgggccgcaggaccgggcgcggagcctcgcagggtcgggctcgggctccctaaccgtctccgcttcttccgccagtcggtggccgggctggcggcgcggttgcagcggcagttcgtggtgcgggcctggggctgcgcgggcccttgcggccgggcagtctttctggccttcgggctagggctgggcctcatcgaggaaaaacaggcggagagccggcgggcggtctcggcctgtcaggagatccaggcaatttttacccagaaaagcaagccggggcctgacccgttggacacgagacgcttgcagggctttcggctggaggagtatctgatagggcagtccattggtaagggctgcagtgctgctgtgtatgaagccaccatgcctacattgccccagaacctggaggtgacaaagagcaccgggttgcttccagggagaggcccaggtaccagtgcaccaggagaagggcaggagcgagctccgggggcccctgccttccccttggccatcaagatgatgtggaacatctcggcaggttcctccagcgaagccatcttgaacacaatgagccaggagctggtcccagcgagccgagtggccttggctggggagtatggagcagtcacttacagaaaatccaagagaggtcccaagcaactagcccctcaccccaacatcatccgggttctccgcgccttcacctcttccgtgccgctgctgccaggggccctggtcgactaccctgatgtgctgccctcacgcctccaccctgaaggcctgggccatggccggacgctgttcctcgttatgaagaactatccctgtaccctgcgccagtacctttgtgtgaacacacccagcccccgcctcgccgccatgatgctgctgcagctgctggaaggcgtggaccatctggttcaacagggcatcgcgcacagagacctgaaatccgacaacatccttgtggagctggacccagacggctgcccctggctggtgatcgcagattttggctgctgcctggctgatgagagcatcggcctgcagttgcccttcagcagctggtacgtggatcggggcggaaacggctgtctgatggccccagaggtgtccacggcccgtcctggccccagggcagtgattgactacagcaaggctgatgcctgggcagtgggagccatcgcctatgaaatcttcgggcttgtcaatcccttctacggccagggcaaggcccaccttgaaagccgcagctaccaagaggctcagctacctgcactgcccgagtcagtgcctccagacgtgagacagttggtgagggcactgctccagcgagaggccagcaagagaccatctgcccgagtagccgcaaatgtgcttcatctaagcctctggggtgaacatattctagccctgaagaatctgaagttagacaagatggttggctggctcctccaacaatcggccgccactttgttggccaacaggctcacagagaagtgttgtgtggaaacaaaaatgaagatgctctttctggctaacctggagtgtgaaacgctctgccaggcagccctcctcctctgctcatggagggcagccctgtgatgtccctgcatggagctggtgaattactaaaagaacatggcatcctctgtgtcgtgatggtctgtgaatggtgagggtgggagtcaggagacaagacagcgcagagagggctggttagccggaaaaggcctcgggcttggcaaatggaagaacttgagtgagagttcagtctgcagtcctctgctcacagacatctgaaaagtgaatggccaagctggtctagtagatgaggctggactgaggaggggtaggcctgcatccacagagaggatccaggccaaggcactggctgtcagtggcagagtttggctgtgacctttgcccctaacacgaggaactcgtttgaagggggcagcgtagcatgtctgatttgccacctggatgaaggcagacatcaacatgggtcagcacgttcagttacgggagtgggaaattacatgaggcctgggcctctgcgttcccaagctgtgcgttctggaccagctactgaattattaatctcacttagcgaaagtgacggatgagcagtaagtaagtaagtgtggggatttaaacttgagggtttccctcctgactagcctctcttacaggaattgtgaaatattaaatgcaaatttacaactgcagatgacgtatgtgccttgaactgaatatttggctttaagaatgattcttatactctgaaggtgagaatattttgtgggcaggtatcaacattggggaagagatttcatgtctaactaactaactttatacatgatttttaggaagctattgcctaaatcagcgtcaacatgcagtaaaggttgtcttcaactga

SEQ ID NO: 50 - DNA, 2291 bp, Homo sapiens
ggaattacttgcagggctaacctagtgcctatagctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 51 - DNA, 2344 bp, Homo sapiens
atcctgccttcagagtcttactgcgcggggccccagtctccagtcccgcccaggcgcctttgcaggctgcggtgggatttcgttttgcctccggttggggctgctgtttctcttcgccgacgtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 52 - DNA, 2325 bp, Homo sapiens
atcctgccttcagagtcttactgcgcggggccccagtctccagtcccgcccaggcgcctttgcaggctgcggtgggatttcgttttgcctccggttggggctgctgtttctcttcgccgacgagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 53 - DNA, 2161 bp, Homo sapiens
atcctgccttcagagtcttactgcgcggggccccagtctccagtcccgcccaggcgcctttgcaggctgcggtgggatttcgttttgcctccggttggggctgctgtttctcttcgccgacggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 54 - DNA, 2144 bp, Homo sapiens
ggaattacttgcagggctaacctagtgcctatagctaaggcaggtacctgcatccttgtttttgtttagtggatcctctatccttcagagactctggaacccctgtggtcttctcttcatctaatgaccctgaggggatggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagccca

SEQ ID NO: 55 - DNA, 124 bp, Artificial Sequence
  source: /note=“Description of Artificial Sequence: Synthetic polynucleotide”
atatgctaatacgactcactataggtgccctctgatgtttttatccccattctacagcagtactgagcaatgccgtagtcagcaatctttgcaatgatggcagcattgggatacagtgtgaaaa

SEQ ID NO: 56 - DNA, 169 bp, Artificial Sequence
  source: /note=“Description of Artificial Sequence: Synthetic polynucleotide”
atatgctaatacgactcactataggtgccctctgatgtttttatccccattctacagcagtactgagcaatgccgtagtcagcaatctttgcaatgatggcagcattgggatacagtgtgaaaagtggaatagtataacaatatgctaaatgttgttatagtatcccac

SEQ ID NO: 57 - DNA, 214 bp, Artificial Sequence
  source: /note=“Description of Artificial Sequence: Synthetic polynucleotide”
atatgctaatacgactcactataggtggaatagtataacaatatgctaaatgttgttatagtatcccacgtgccctctgatgtttttatccccattctacagcagtactgagcaatgccgtagtcagcaatctttgcaatgatggcagcattgggatacagtgtgaaaagtggaatagtataacaatatgctaaatgttgttatagtatcccac

SEQ ID NO: 58 - DNA, 124 bp, Artificial Sequence
  source: /note=“Description of Artificial Sequence: Synthetic polynucleotide”
atatgctaatacgactcactataggtgccctctgatgtttttatccccattcggttacagtaatgagcaaatgccgagtcagcaagatttgctggttgggcagcattgggatacagtgtgaaaa

SEQ ID NO: 59 - DNA, 111 bp, Artificial Sequence
  source: /note=“Description of Artificial Sequence: Synthetic polynucleotide”
atatgctaatacgactcactatagaaggacgggtcgtactgagcaatgccctagtcaaagtggacagcaatctttgcaacgatggcagcatcgggatacacctgtgactaa

SEQ ID NO: 60 - DNA, 169 bp, Artificial Sequence
  source: /note=“Description of Artificial Sequence: Synthetic polynucleotide”
atatgctaatacgactcactataggtgccctctgatgttcttatccccattccacagcagtactgagcaatgccgtagtcacaccctatgatattgttgtaaatcgtataacaatatgataaggtggcaatcttcgcaatgatggcagcattgggacacagtgtgaaaa

SEQ ID NO: 61 - DNA, 169 bp, Artificial Sequence
  source: /note=“Description of Artificial Sequence: Synthetic polynucleotide”
atatgctaatacgactcactataggtgccctctgatgttcttatccccattccacagcagtactgagcaatgccgtagtcagtggaatagtataacaatatgctaaatgttgttatagtatcccacgcaatcttcgcaatgatggcagcattgggatacagtgtgaaaa

SEQ ID NO: 62 - DNA, 111 bp, Artificial Sequence
  source: /note=“Description of Artificial Sequence: Synthetic polynucleotide”
atatgctaatacgactcactatagaaggacgggtcgtactgagcaatgccgtagtcaaagtggacagcaatctttgcaacgatggcagcatcgggatacacctgtgactaa

SEQ ID NO: 63 - DNA, 21 bp, Artificial Sequence
  source: /note=“Description of Artificial Sequence: Synthetic primer”
ttttcacactgtatcccaatg

SEQ ID NO: 64 - DNA, 19 bp, Artificial Sequence
  source: /note=“Description of Artificial Sequence: Synthetic primer”
ttagtcacaggtgtatccc

SEQ ID NO: 65 - DNA, 21 bp, Artificial Sequence
  source: /note=“Description of Artificial Sequence: Synthetic primer”
ttttcacactgtatcccaatg

SEQ ID NO: 66 - RNA, 100 nt, Artificial Sequence
  source: /note=“Description of Artificial Sequence: Synthetic polynucleotide”
gugcccucugauguuuuuauccccauucuacagcaguacugagcaaugccguagucagcaaucuuugcaaugauggcagcauugggauacagugugaaaa

SEQ ID NO: 67 - RNA, 145 nt, Artificial Sequence
  source: /note=“Description of Artificial Sequence: Synthetic polynucleotide”
gugcccucugauguuuuuauccccauucuacagcaguacugagcaaugccguagucagcaaucuuugcaaugauggcagcauugggauacagugugaaaaguggaauaguauaacaauaugcuaaauguuguuauaguaucccac

SEQ ID NO: 68 - RNA, 190 nt, Artificial Sequence
  source: /note=“Description of Artificial Sequence: Synthetic polynucleotide”
guggaauaguauaacaauaugcuaaauguuguuauaguaucccacgugcccucugauguuuuuauccccauucuacagcaguacugagcaaugccguagucagcaaucuuugcaaugauggcagcauugggauacagugugaaaaguggaauaguauaacaauaugcuaaauguuguuauaguaucccac

SEQ ID NO: 69 - RNA, 100 nt, Artificial Sequence
  source: /note=“Description of Artificial Sequence: Synthetic polynucleotide”
gugcccucugauguuuuuauccccauucgguuacaguaaugagcaaaugccgagucagcaagauuugcugguugggcagcauugggauacagugugaaaa

SEQ ID NO: 70 - RNA, 87 nt, Artificial Sequence
  source: /note=“Description of Artificial Sequence: Synthetic oligonucleotide”
aaggacgggucguacugagcaaugcccuagucaaaguggacagcaaucuuugcaacgauggcagcaucgggauacaccugugacuaa

SEQ ID NO: 71 - RNA, 145 nt, Artificial Sequence
  source: /note=“Description of Artificial Sequence: Synthetic polynucleotide”
gugcccucugauguucuuauccccauuccacagcaguacugagcaaugccguagucacacccuaugauauuguuguaaaucguauaacaauaugauaagguggcaaucuucgcaaugauggcagcauugggacacagugugaaaa

SEQ ID NO: 72 - RNA, 145 nt, Artificial Sequence
  source: /note=“Description of Artificial Sequence: Synthetic polynucleotide”
gugcccucugauguucuuauccccauuccacagcaguacugagcaaugccguagucaguggaauaguauaacaauaugcuaaauguuguuauaguaucccacgcaaucuucgcaaugauggcagcauugggauacagugugaaaa

SEQ ID NO: 73 - RNA, 100 nt, Artificial Sequence
  source: /note=“Description of Artificial Sequence: Synthetic polynucleotide”
uuuucacacuguaucccaaugcugccaucauugcaaagauugcugacuacagcauugcucaguacugcuguagaauggggauaaaaacaucagagggcac

SEQ ID NO: 74 - RNA, 56 nt, Artificial Sequence
  source: /note=“Description of Artificial Sequence: Synthetic oligonucleotide”
uguaucccaaugcugccaucauugcaaagauugcugacuacagcauugcucaguac

SEQ ID NO: 75 - DNA, 24 bp, Artificial Sequence
  source: /note=“Description of Artificial Sequence: Synthetic primer”
cgtagctgatggtttgagatacct

SEQ ID NO: 76 - DNA, 27 bp, Artificial Sequence
  source: /note=“Description of Artificial Sequence: Synthetic primer”
accaaatgaataaacatcagcctgttg

SEQ ID NO: 77 - DNA, 22 bp, Artificial Sequence
  source: /note=“Description of Artificial Sequence: Synthetic primer”
tttcctctggcaacttcaggtg

SEQ ID NO: 78 - DNA, 5414 bp, Artificial Sequence
  source: /note=“Description of Artificial Sequence: Synthetic polynucleotide”
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgccattgggatgttgtaaaacgacggccagtgaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaccgagagaccagctggatggtctcagtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctagcgtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttggaattcatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttaagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtgaaccggtcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcttggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 79 - DNA, 5288 bp, Artificial Sequence
  source: /note=“Description of Artificial Sequence: Synthetic polynucleotide”
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgccattgggatgttgtaaaacgacggccagtgaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggaggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagagaccagctggatggtctcttttttttcgtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttggatgtcttcacaggaagacgctttttttgcggccgcatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagacaccgggaccgatccagcctccggactctagaggatcgaacccttaagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtgaaccggtcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcttggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 80 - DNA, 5456 bp, Artificial Sequence
  source: /note=“Description of Artificial Sequence: Synthetic polynucleotide”
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgccattgggatgttgtaaaacgacggccagtgaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggaggagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggccatcagtcgccggtcccaagcccggataaaatgggagggggcgggaaaccgcctaaccatgccgactgatggcagataatagagaccagctggatggtctcaatatactgccatcagtcggcgtggactgtagaacactgccaatgccggtcccaagcccggataaaagtggagggtacagtccacgctttttttcgtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttggatgtcttcacaggaagacgctttttttgcggccgcatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagacaccgggaccgatccagcctccggactctagaggatcgaacccttaagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtgaaccggtcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcttggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 81 - DNA, 145 bp, Artificial Sequence
  source: /note=“Description of Artificial Sequence: Synthetic polynucleotide”
gtgccctctgatgtttttatccccattctacagcattacggagcagtgccgtagtgtcgtttctttgcaatgatggcagcattggatacagtgtgaaaagtggaatttttggagcaggttttctgacttcggtcggaaaacccct

SEQ ID NO: 82 - DNA, 146 bp, Artificial Sequence
  source: /note=“Description of Artificial Sequence: Synthetic polynucleotide”
ctggcaacttcaggtgcacgaaaccctggtgtgccctctgatgtttttatccccattctacagcattacggagcagtgccgtagtgtcgtttctttgcaagtggaatttttggagcaggttttctgacttcggtcggaaaacccct

SEQ ID NO: 83 - DNA, 100 bp, Artificial Sequence
  source: /note=“Description of Artificial Sequence: Synthetic polynucleotide”
ctggcaacttcaggtgcacgaaaccctggtgtgccctctgatgttcttatccccattctacagcagtactgagcaatgccgtagtcagcaatctttgcaa

SEQ ID NO: 84 - DNA, 45 bp, Artificial Sequence
  source: /note=“Description of Artificial Sequence: Synthetic oligonucleotide”
tacagcagtactgagcaatgctgtagtcagcaatctttgcaatga

SEQ ID NO: 85 - DNA, 45 bp, Artificial Sequence
  source: /note=“Description of Artificial Sequence: Synthetic oligonucleotide”
tacagcagtactgagcaatgccgtagtcagcaatctttgcaatga

SEQ ID NO: 86 - DNA, 45 bp, Artificial Sequence
  source: /note=“Description of Artificial Sequence: Synthetic oligonucleotide”
tacagcagtactgggtggtgccgtagtcagcaatctttgcaatga

SEQ ID NO: 87 - DNA, 45 bp, Artificial Sequence
  source: /note=“Description of Artificial Sequence: Synthetic oligonucleotide”
tacagcaggactgagcagtgccgtagtgtcgaatctttgcaatga

SEQ ID NO: 88 - DNA, 45 bp, Artificial Sequence
  source: /note=“Description of Artificial Sequence: Synthetic oligonucleotide”
taaagcaggactgagtagtgccgtagtcagcaatctttgcaatga

SEQ ID NO: 89 - DNA, 45 bp, Artificial Sequence
  source: /note=“Description of Artificial Sequence: Synthetic oligonucleotide”
tacagcagtactgagcagtgccgtagtgtcgtttctttgcaatga

SEQ ID NO: 90 - DNA, 45 bp, Artificial Sequence
  source: /note=“Description of Artificial Sequence: Synthetic oligonucleotide”
tacagcattactgagcagtgccgtagtgtcgtttctttgcaatga

SEQ ID NO: 91 - DNA, 45 bp, Artificial Sequence
  source: /note=“Description of Artificial Sequence: Synthetic oligonucleotide”
tacagcaggactgagtagtgccgtagtgtcgaatctttgcaatga

SEQ ID NO: 92 - DNA, 45 bp, Artificial Sequence
  source: /note=“Description of Artificial Sequence: Synthetic oligonucleotide”
tacagcaggactgagcggtgccgtagtcagcaatctttgcaatga

SEQ ID NO: 93 - DNA, 45 bp, Artificial Sequence
  source: /note=“Description of Artificial Sequence: Synthetic oligonucleotide”
tacagcagtactgagcggtgccgtagtcagcaatctttgcaatga

SEQ ID NO: 94 - DNA, 45 bp, Artificial Sequence
  source: /note=“Description of Artificial Sequence: Synthetic oligonucleotide”
tacagcaggactgggtgatgccgtagtcagcaatctttgcaatga

SEQ ID NO: 95 - DNA, 45 bp, Artificial Sequence
  source: /note=“Description of Artificial Sequence: Synthetic oligonucleotide”
taaagcagtactgagcggtgccgtagtcagcaatctttgcaatga

SEQ ID NO: 96 - DNA, 45 bp, Artificial Sequence
  source: /note=“Description of Artificial Sequence: Synthetic oligonucleotide”
tacagcagtcctgggtggtgccgtagtcagcaatctttgcaatga

SEQ ID NO: 97 - DNA, 45 bp, Artificial Sequence
  source: /note=“Description of Artificial Sequence: Synthetic oligonucleotide”
tacagcaggactgggtagtgccgtagtgtcgaatctttgcaatga

SEQ ID NO: 98 - DNA, 45 bp, Artificial Sequence
  source: /note=“Description of Artificial Sequence: Synthetic oligonucleotide”
tacagcaggactgagcagtgccgtagtcagcaatctttgcaatga

SEQ ID NO: 99 - DNA, 45 bp, Artificial Sequence
  source: /note=“Description of Artificial Sequence: Synthetic oligonucleotide”
taaagcagtactgagcagtgccgtagtcagcaatctttgcaatga

SEQ ID NO: 100 - DNA, 45 bp, Artificial Sequence
  source: /note=“Description of Artificial Sequence: Synthetic oligonucleotide”
taaagcaggcctgagcagtgccgtagtgtcgaatctttgcaatga

SEQ ID NO: 101 - DNA, 45 bp, Artificial Sequence
  source: /note=“Description of Artificial Sequence: Synthetic oligonucleotide”
taaagcaggactgggcagtgccgtagtgtcgaatctttgcaatga

SEQ ID NO: 102 - DNA, 45 bp, Artificial Sequence
  source: /note=“Description of Artificial Sequence: Synthetic oligonucleotide”
tacagcaggactgggcgatgccgtagtcagcaatctttgcaatga

SEQ ID NO: 103 - DNA, 45 bp, Artificial Sequence
  source: /note=“Description of Artificial Sequence: Synthetic oligonucleotide”
tacagcaggactgggcagtgccgtagtcagcaatctttgcaatga

SEQ ID NO: 104 - DNA, 45 bp, Artificial Sequence
  source: /note=“Description of Artificial Sequence: Synthetic oligonucleotide”
taaagcaggactgggcagtgccgtagtcagcaatctttgcaatga

SEQ ID NO: 105 - DNA, 45 bp, Artificial Sequence
  source: /note=“Description of Artificial Sequence: Synthetic oligonucleotide”
tacagcattacggagcagtgccgtagtgtcgtttctttgcaatga

SEQ ID NO: 106 - DNA, 45 bp, Artificial Sequence
  source: /note=“Description of Artificial Sequence: Synthetic oligonucleotide”
tacagcaggcctgagcagtgccgtagtgtcgaatctttgcaatga

SEQ ID NO: 107 - DNA, 45 bp, Artificial Sequence
  source: /note=“Description of Artificial Sequence: Synthetic oligonucleotide”
taaagcaggactgggtagtgccgtagtgtcgaatctttgcaatga

SEQ ID NO: 108 - DNA, 45 bp, Artificial Sequence
  source: /note=“Description of Artificial Sequence: Synthetic oligonucleotide”
tacagcagtactgagcagtgccgtagtgtcgaatctttgcaatga

SEQ ID NO: 109 - DNA, 45 bp, Artificial Sequence
  source: /note=“Description of Artificial Sequence: Synthetic oligonucleotide”
tacagcaataccgagcagtgccgtagtcagcaatctttgcaatga

SEQ ID NO: 110 - DNA, 45 bp, Artificial Sequence
  source: /note=“Description of Artificial Sequence: Synthetic oligonucleotide”
tacagcagtactgggcggtgccgtagtcagcaatctttgcaatga

SEQ ID NO: 111 - DNA, 45 bp, Artificial Sequence
  source: /note=“Description of Artificial Sequence: Synthetic oligonucleotide”
tacagcaggactgggtggtgccgtagtcagcaatctttgcaatga

SEQ ID NO: 112 - DNA, 45 bp, Artificial Sequence
  source: /note=“Description of Artificial Sequence: Synthetic oligonucleotide”
taaagcaggactgagcagtgccgtagtcagcaatctttgcaatga

SEQ ID NO: 113 - DNA, 45 bp, Artificial Sequence
  source: /note=“Description of Artificial Sequence: Synthetic oligonucleotide”
taaagcaggactgagtagtgccgtagtgtcgaatctttgcaatga

SEQ ID NO: 114 - DNA, 45 bp, Artificial Sequence
  source: /note=“Description of Artificial Sequence: Synthetic oligonucleotide”
tacagcagtactgagtggtgccgtagtcagcaatctttgcaatga

SEQ ID NO: 115 - DNA, 45 bp, Artificial Sequence
  source: /note=“Description of Artificial Sequence: Synthetic oligonucleotide”
tacagcaggactgggcggtgccgtagtcagcaatctttgcaatga

SEQ ID NO: 116 - DNA, 45 bp, Artificial Sequence
  source: /note=“Description of Artificial Sequence: Synthetic oligonucleotide”
tacagcagtcctgggcggtgccgtagtcagcaatctttgcaatga

SEQ ID NO: 117 - DNA, 45 bp, Artificial Sequence
  source: /note=“Description of Artificial Sequence: Synthetic oligonucleotide”
taaagcagtactgagcagtgccgtagtgtcgaatctttgcaatga

SEQ ID NO: 118 - DNA, 45 bp, Artificial Sequence
  source: /note=“Description of Artificial Sequence: Synthetic oligonucleotide”
taaagcaggactgagcagtgccgtagtgtcgaatctttgcaatga

SEQ ID NO: 119 - DNA, 45 bp, Artificial Sequence
  source: /note=“Description of Artificial Sequence: Synthetic oligonucleotide”
tacagcaggactgggcagtgccgtagtgtcgaatctttgcaatga

SEQ ID NO: 120 - DNA, 63 bp, Artificial Sequence
  source: /note=“Description of Artificial Sequence: Synthetic oligonucleotide”
gctaaatcgattctaaagcaggacggggtggtgccgtagtgtcgtatctttgcacttacagtc

SEQ ID NO: 121 - DNA, 63 bp, Artificial Sequence
  source: /note=“Description of Artificial Sequence: Synthetic oligonucleotide”
gctaaatcgattctaaagcaggactgggtggtgccgtagtgtcgtatctttgcacttacagtc

SEQ ID NO: 122 - DNA, 63 bp, Artificial Sequence
  source: /note=“Description of Artificial Sequence: Synthetic oligonucleotide”
gctaaatcgattctaaagcaggaccgggtggtgccgtagtgtcgtatctttgcacttacagtc

SEQ ID NO: 123 - DNA, 63 bp, Artificial Sequence
  source: /note=“Description of Artificial Sequence: Synthetic oligonucleotide”
gctaaatcgattctagagcaggactgggtggtgccgtagtgtcgtatctttgcacttacagtc

SEQ ID NO: 124 - DNA, 63 bp, Artificial Sequence
  source: /note=“Description of Artificial Sequence: Synthetic oligonucleotide”
gctaaatcgattctacagcaggactgggtggtgccgtagtgtcgtatctttgcacttacagtc

SEQ ID NO: 125 - DNA, 63 bp, Artificial Sequence
  source: /note=“Description of Artificial Sequence: Synthetic oligonucleotide”
gctaaatcgattctaaagcatgactgggtggtgccgtagtcagcaatctttgcacttacagtc

SEQ ID NO: 126 - DNA, 63 bp, Artificial Sequence
  source: /note=“Description of Artificial Sequence: Synthetic oligonucleotide”
gctaaatcgattctaaagcaggactgggtggtgccgtagtcagcaatctttgcacttacagtc

SEQ ID NO: 127 - DNA, 63 bp, Artificial Sequence
  source: /note=“Description of Artificial Sequence: Synthetic oligonucleotide”
gctaaatcgattctaaagcagtactgggtggtgccgtagtcagcaatctttgcacttacagtc

SEQ ID NO: 128 - DNA, 63 bp, Artificial Sequence
  source: /note=“Description of Artificial Sequence: Synthetic oligonucleotide”
gctaaatcgattctagagcatgactgggtggtgccgtagtcagcaatctttgcacttacagtc

SEQ ID NO: 129 - DNA, 63 bp, Artificial Sequence
  source: /note=“Description of Artificial Sequence: Synthetic oligonucleotide”
gctaaatcgattctagagcaggacggggtggtgccgtagtcagcaatctttgcacttacagtc

SEQ ID NO: 130 - DNA, 113 bp, Artificial Sequence
  source: /note=“Description of Artificial Sequence: Synthetic polynucleotide”
ccctggtgtgccctctgatgttttttaggggattctacagtaggactgagcactgccgagctgggcaatctttgcatactacgcagcattgggatacagtgtgaaaagcagca

SEQ ID NO: 131 - DNA, 113 bp, Artificial Sequence
  source: /note=“Description of Artificial Sequence: Synthetic polynucleotide”
ccctggtgtgccctctgatgttttttaggggattctacagcagtacagaggactgccgaggtcaccaatctttgcatactacgcagcattgggatacagtgtgaaaagcagca

SEQ ID NO: 132 - DNA, 113 bp, Artificial Sequence
  source: /note=“Description of Artificial Sequence: Synthetic polynucleotide”
ccctggtgtgccctctgatgttttttaggggattctacaacagtactgagctatcccgaattcaacaatctttgcatactacgcagcattgggatacagtgtgaaaagcagca

SEQ ID NO: 133 - DNA, 113 bp, Artificial Sequence
  source: /note=“Description of Artificial Sequence: Synthetic polynucleotide”
ccctggtgtgccctctgatgttttttaggggcttctacagcagttcggaggaatcccgaggtcagcaatctttgcatactacgcagcattgggatacagtgtgaaaagcagca

SEQ ID NO: 134 - DNA, 112 bp, Artificial Sequence
  source: /note=“Description of Artificial Sequence: Synthetic polynucleotide”
ccctggtgtgccctctgatgttttttaggggattctacggcggtactgaccaatcccgtagttagcaatctttgcatactacgcagcattgggatacagtgtgaaaagcagc

SEQ ID NO: 135 - DNA, 113 bp, Artificial Sequence
  source: /note=“Description of Artificial Sequence: Synthetic polynucleotide”
ccctggtgtgccctctgatgttttttaggggattctaccgcagtactacccgatcccgtagtcagcaatctttgcatactacgcagcattgggatacagtgtgaaaagcagca

SEQ ID NO: 136 - DNA, 113 bp, Artificial Sequence
  source: /note=“Description of Artificial Sequence: Synthetic polynucleotide”
ccctggtgtgccctctgatgttttttaggggattctacggcagttcatagcaatcccgtagtcaacaatctttgcctactacgcagcattgggatacagtgtgaaaagcagca

SEQ ID NO: 137 - DNA, 45 bp, Artificial Sequence
  source: /note=“Description of Artificial Sequence: Synthetic oligonucleotide”
attctacagcacgactgagcaatgccgtattcagcaatctttgca

SEQ ID NO: 138 - DNA, 45 bp, Artificial Sequence
  source: /note=“Description of Artificial Sequence: Synthetic oligonucleotide”
attctagagcagtactgagcaatgccgtggttaccaatctttgca

SEQ ID NO: 139 - DNA, 45 bp, Artificial Sequence
  source: /note=“Description of Artificial Sequence: Synthetic oligonucleotide”
attctacagcagtagtaagctttgccgaagtgagcaatctttaca

SEQ ID NO: 140 - DNA, 45 bp, Artificial Sequence
  source: /note=“Description of Artificial Sequence: Synthetic oligonucleotide”
attctacagcacgactgagcaaggccgtagtcagcgatctttgca

SEQ ID NO: 141 - DNA, 45 bp, Artificial Sequence
  source: /note=“Description of Artificial Sequence: Synthetic oligonucleotide”
attctacagcactacggggcaatgccgaagtctgcaatctttgca

SEQ ID NO: 142 - DNA, 45 bp, Artificial Sequence
  source: /note=“Description of Artificial Sequence: Synthetic oligonucleotide”
attctacggcggtactgaccaatcccgtagttagcaatctttgca

SEQ ID NO: 143 - DNA, 45 bp, Artificial Sequence
  source: /note=“Description of Artificial Sequence: Synthetic oligonucleotide”
attctacagcggtactcatcaatgccgtagtcgtcaatttttgca

SEQ ID NO: 144 - DNA, 45 bp, Artificial Sequence
  source: /note=“Description of Artificial Sequence: Synthetic oligonucleotide”
attctaaagccgtactaagcagtaccgtagtccccaatctttgca

SEQ ID NO: 145 - DNA, 45 bp, Artificial Sequence
  source: /note=“Description of Artificial Sequence: Synthetic oligonucleotide”
attctagagccatactgttcaatgccgaagtcagcaatctttgca

SEQ ID NO: 146 - DNA, 45 bp, Artificial Sequence
  source: /note=“Description of Artificial Sequence: Synthetic oligonucleotide”
attctacagcggtactgggcaatgccgtggtctgcaatctttgca

SEQ ID NO: 147 - DNA, 45 bp, Artificial Sequence
  source: /note=“Description of Artificial Sequence: Synthetic oligonucleotide”
attctacagctgtactgaggaatgccgtctttaccaatctttgca

SEQ ID NO: 148 - DNA, 45 bp, Artificial Sequence
  source: /note=“Description of Artificial Sequence: Synthetic oligonucleotide”
attctacagcagtacttatcagtgccgttgttagcaatctttgca

SEQ ID NO: 149 - DNA, 45 bp, Artificial Sequence
  source: /note=“Description of Artificial Sequence: Synthetic oligonucleotide”
attctatagctggactgagctctgccgtagtcgtcaatctttgca

SEQ ID NO: 150 - DNA, 45 bp, Artificial Sequence
  source: /note=“Description of Artificial Sequence: Synthetic oligonucleotide”
attctacagcaatactcagcagtgccgtagtcaccaatctttgca

SEQ ID NO: 151 - DNA, 45 bp, Artificial Sequence
  source: /note=“Description of Artificial Sequence: Synthetic oligonucleotide”
attctacaacagtactgcgcaccgccgtagtctgcaatctttgca

SEQ ID NO: 152 - DNA, 45 bp, Artificial Sequence
  source: /note=“Description of Artificial Sequence: Synthetic oligonucleotide”
attctagagccgtactgcgctttgccgtagtcatcaatctttgca

SEQ ID NO: 153 - DNA, 45 bp, Artificial Sequence
  source: /note=“Description of Artificial Sequence: Synthetic oligonucleotide”
attctacagccgtactgcccaatgccgaagtaaccaatctttgca

SEQ ID NO: 154 - DNA, 45 bp, Artificial Sequence
  source: /note=“Description of Artificial Sequence: Synthetic oligonucleotide”
attctaccgtgggactgagcagtcccgtagtctgcaatctttgca

SEQ ID NO: 155 - DNA, 45 bp, Artificial Sequence
  source: /note=“Description of Artificial Sequence: Synthetic oligonucleotide”
attctacagcagtactgttcagtgcgctagtaagcaatctttgca

SEQ ID NO: 156 - DNA, 45 bp, Artificial Sequence
  source: /note=“Description of Artificial Sequence: Synthetic oligonucleotide”
attctacaggtgtactgttctatgccgttgttagccatctttgca

SEQ ID NO: 157 - DNA, 45 bp, Artificial Sequence
  source: /note=“Description of Artificial Sequence: Synthetic oligonucleotide”
attctagggcagtactgagagatgccgatgtcagcaatctttgca

SEQ ID NO: 158 - DNA, 45 bp, Artificial Sequence
  source: /note=“Description of Artificial Sequence: Synthetic oligonucleotide”
attctacagcagtattgggcagtcccgtagtcagcaatctttgca

SEQ ID NO: 159 - DNA, 45 bp, Artificial Sequence
  source: /note=“Description of Artificial Sequence: Synthetic oligonucleotide”
attctacagcaggactgggcgatcccgtagtcatcaatctttgca

SEQ ID NO: 160 - DNA, 45 bp, Artificial Sequence
  source: /note=“Description of Artificial Sequence: Synthetic oligonucleotide”
attctacagcagtactaagctatggcgtacgcaccaatctttgca

SEQ ID NO: 161 - DNA, 45 bp, Artificial Sequence
  source: /note=“Description of Artificial Sequence: Synthetic oligonucleotide”
attctacagtaggactgagcactgccgagctgggcaatctttgca

SEQ ID NO: 162 - DNA, 45 bp, Artificial Sequence
  source: /note=“Description of Artificial Sequence: Synthetic oligonucleotide”
attctaccgcagtactgagcaaagccgaggtaaacaatctttgca

SEQ ID NO: 163 - DNA, 45 bp, Artificial Sequence
  source: /note=“Description of Artificial Sequence: Synthetic oligonucleotide”
attctacagcagtactgagcaattccgttaaccgcaatctttgca

SEQ ID NO: 164 - DNA, 45 bp, Artificial Sequence
  source: /note=“Description of Artificial Sequence: Synthetic oligonucleotide”
attctacagtagtccggagctattccgtagtccgcaatctttgca

SEQ ID NO: 165 - DNA, 45 bp, Artificial Sequence
  source: /note=“Description of Artificial Sequence: Synthetic oligonucleotide”
attctacagcaggactgagcaatggagtagtcggcaatctttgca

SEQ ID NO: 166 - DNA, 45 bp, Artificial Sequence
  source: /note=“Description of Artificial Sequence: Synthetic oligonucleotide”
attctaccgcagtactacccgatcccgtagtcagcaatctttgca

SEQ ID NO: 167 - DNA, 45 bp, Artificial Sequence
  source: /note=“Description of Artificial Sequence: Synthetic oligonucleotide”
attctacaggagttctgagcattcccgtcgtcatcaatctttgca

SEQ ID NO: 168 - DNA, 45 bp, Artificial Sequence
  source: /note=“Description of Artificial Sequence: Synthetic oligonucleotide”
attctacagcagtacagaggactgccgaggtcaccaatctttgca

SEQ ID NO: 169 - DNA, 45 bp, Artificial Sequence
  source: /note=“Description of Artificial Sequence: Synthetic oligonucleotide”
attctacagaaggaccgagcagtcccgtagtcagcaatctttgca

SEQ ID NO: 170 - DNA, 45 bp, Artificial Sequence
  source: /note=“Description of Artificial Sequence: Synthetic oligonucleotide”
attctacagcattactgagcaatcccgtattaagcaatctttgca

SEQ ID NO: 171 - DNA, 45 bp, Artificial Sequence
  source: /note=“Description of Artificial Sequence: Synthetic oligonucleotide”
attctacagcagtacgaagcaatcccgccgttagcaatctttgca

SEQ ID NO: 172 - DNA, 45 bp, Artificial Sequence
  source: /note=“Description of Artificial Sequence: Synthetic oligonucleotide”
attctacagcagtactgttcaattccgatgtaagcaatctttgca

SEQ ID NO: 173 - DNA, 45 bp, Artificial Sequence
  source: /note=“Description of Artificial Sequence: Synthetic oligonucleotide”
attctacaacagtactgagctatcccgaattcaacaatctttgca

SEQ ID NO: 174 - DNA, 45 bp, Artificial Sequence
  source: /note=“Description of Artificial Sequence: Synthetic oligonucleotide”
attctacggcagttcatagcaatcccgtagtcaacaatctttgcc

SEQ ID NO: 175 - DNA, 45 bp, Artificial Sequence
  source: /note=“Description of Artificial Sequence: Synthetic oligonucleotide”
cttctacagcagttcggaggaatcccgaggtcagcaatctttgca

SEQ ID NO: 176 - DNA, 45 bp, Artificial Sequence
  source: /note=“Description of Artificial Sequence: Synthetic oligonucleotide”
attctacagctgtactaggctatcccgtagtccgcaatctttgca

SEQ ID NO: 177 - DNA, 45 bp, Artificial Sequence
  source: /note=“Description of Artificial Sequence: Synthetic oligonucleotide”
attctacagccgtactgggaaatcccgtagtctgcaatctttgca

SEQ ID NO: 178 - DNA, 45 bp, Artificial Sequence
  source: /note=“Description of Artificial Sequence: Synthetic oligonucleotide”
attctacagaagtacagagacatcccgtagttaacaatcttcgca

SEQ ID NO: 179 - DNA, 45 bp, Artificial Sequence
  source: /note=“Description of Artificial Sequence: Synthetic oligonucleotide”
attctacagccgtactgagctatcccgtagtccgcaatctttgca

SEQ ID NO: 180 - DNA, 45 bp, Artificial Sequence
  source: /note=“Description of Artificial Sequence: Synthetic oligonucleotide”
attctactgcagcaccgagcaatcccgcattccccaatctttgca

SEQ ID NO: 181 - DNA, 45 bp, Artificial Sequence
  source: /note=“Description of Artificial Sequence: Synthetic oligonucleotide”
attctacagcagtcctgagcagtaccgtagtcagcaatctttgca

SEQ ID NO: 182 - DNA, 45 bp, Artificial Sequence
  source: /note=“Description of Artificial Sequence: Synthetic oligonucleotide”
attctaaggcagtactgagccgcgccgtagtcggcaatctttgca

SEQ ID NO: 183 - DNA, 45 bp, Artificial Sequence
  source: /note=“Description of Artificial Sequence: Synthetic oligonucleotide”
gaaagtactttgatgaatacatccacggctaatgaattcctttac

SEQ ID NO: 184 - DNA, 45 bp, Artificial Sequence
  source: /note=“Description of Artificial Sequence: Synthetic oligonucleotide”
gaaagtcctttcaagaatacatccacggctatactattcctttac

SEQ ID NO: 185 - DNA, 45 bp, Artificial Sequence
  source: /note=“Description of Artificial Sequence: Synthetic oligonucleotide”
gaaagtcctttgatgcatacatccacggctaatgaattcctttac

SEQ ID NO: 186 - DNA, 45 bp, Artificial Sequence
  source: /note=“Description of Artificial Sequence: Synthetic oligonucleotide”
gaaagtcctttgatgcatacatccacggctatactattcctttac

SEQ ID NO: 187 - DNA, 45 bp, Artificial Sequence
  source: /note=“Description of Artificial Sequence: Synthetic oligonucleotide”
gaaagtcctttgaggcatacatccacggctatactattcctttac

SEQ ID NO: 188 - DNA, 45 bp, Artificial Sequence
  source: /note=“Description of Artificial Sequence: Synthetic oligonucleotide”
gaaagtcctttgaggaatacatccacggctatactattcctttac

SEQ ID NO: 189 - DNA, 45 bp, Artificial Sequence
  source: /note=“Description of Artificial Sequence: Synthetic oligonucleotide”
gaaagtcctttgaagcatacatccacggctatactattcctttac

SEQ ID NO: 190 - DNA, 45 bp, Artificial Sequence
  source: /note=“Description of Artificial Sequence: Synthetic oligonucleotide”
gaaagtactttcacgaatacatccacggctatactattcctttac

SEQ ID NO: 191 - DNA, 45 bp, Artificial Sequence; identical to SEQ ID NO: 190
  source: /note=“Description of Artificial Sequence: Synthetic oligonucleotide”

SEQ ID NO: 192 - DNA, 45 bp, Artificial Sequence
  source: /note=“Description of Artificial Sequence: Synthetic oligonucleotide”
gaaagtcctttcaagcatacatccacggctatactattcctttac

SEQ ID NO: 193 - protein, 6 aa, Artificial Sequence
  source: /note=“Description of Artificial Sequence: Synthetic peptide”
  MOD_RES (2)..(2): Any amino acid
KXKEGV

SEQ ID NO: 194 - protein, 9 aa, Artificial Sequence
  source: /note=“Description of Artificial Sequence: Synthetic peptide”
VGGAVVTGV

SEQ ID NO: 195 - protein, 6 aa, Artificial Sequence
  source: /note=“Description of Artificial Sequence: Synthetic peptide”
DPDNEA

SEQ ID NO: 196 - DNA, 200 bp, Artificial Sequence
  source: /note=“Description of Artificial Sequence: Synthetic polynucleotide”
gtgccctctgatgtttttatccccattctacagcagtactgagcaatgccgtagtcagcaatctttgcaatgatggcagcattgggatacagtgtgaaaattttcacactgtatcccaatgctgccatcattgcaaagattgctgactacggcattgctcagtactgctgtagaatggggataaaaacatcagagggcac

SEQ ID NO: 197 - RNA, 165 nt, Artificial Sequence
  source: /note=“Description of Artificial Sequence: Synthetic polynucleotide”
uugugaaagaaggacgggucccuucucuccgucauugcaaagauugcugacuacagcauugcucaguacugcuguaauaugcuaaauguuacagcaggacugggcagugccguagugucgaaucuuugcaaugaccuucucuccgccugugacuaacugugccaa

SEQ ID NO: 198 - RNA, 245 nt, Artificial Sequence
  source: /note=“Description of Artificial Sequence: Synthetic polynucleotide”
gugcccucugauguuuuuauccccauucuacagcaguacugagcaaugccguagucagcaaucuuugcaaugauggcagcauugggauacagugugaaaaguggaauaguauaacaauaugcuaaauguuguuauaguaucccacuuuucacacuguaucccaaugcugccaucauugcaaagauugcugacuacagcauugcucaguacugcuguagaauggggauaaaaacaucagagggcac

SEQ ID NO: 199 - RNA, 290 nt, Artificial Sequence
  source: /note=“Description of Artificial Sequence: Synthetic polynucleotide”
uuuucacacuguaucccaaugcugccaucauugcaaagauugcugacuacagcauugcucaguacugcuguagaauggggauaaaaacaucagagggcacguggaauaguauaacaauaugcuaaauguuguuauaguaucccacgugcccucugauguuuuuauccccauucuacagcaguacugagcaaugccguagucagcaaucuuugcaaugauggcagcauugggauacagugugaaaaguggaauaguauaacaauaugcuaaauguuguuauaguaucccac

SEQ ID NO: 200 - RNA, 246 nt, Artificial Sequence
  source: /note=“Description of Artificial Sequence: Synthetic polynucleotide”
gugcccucugauguucuuauccccauuccacagcaguacugagcaaugccguagucaguggaauaguauaacaauaugcuaaauguuguuauaguaucccacgcaaucuucgcaaugauggcagcauugggauacagugugaaaauuuucacacuguaucccaaugcugccaucauugcgaauauugcugacuacagcauugcucaguacugcugugagaauggggauaaaaacaucagagggcac

SEQ ID NO: 201 - RNA, 45 nt, Artificial Sequence; identical to SEQ ID NO: 1
  source: /note=“Description of Artificial Sequence: Synthetic oligonucleotide”

SEQ ID NO: 202 - RNA, 245 nt, Artificial Sequence
  source: /note=“Description of Artificial Sequence: Synthetic polynucleotide”
gugcccucugauguucuuauccccauuccacagcaguacugagcaaugccguagucacacccuaugauauuguuguaaaucguauaacaauaugauaagguggcaaucuucgcaaugauggcagcauugggacacagugugaaaauuuucacacuguguccaaaugcugccaucauugcgaaaauugcugacuacagcauugcucaguacugcuguagaauggggauaaaaacaucagagggcac

SEQ ID NO: 203 - RNA, 45 nt, Artificial Sequence
  source: /note=“Description of Artificial Sequence: Synthetic oligonucleotide”
cacccuaugauauuguuguaaaucguauaacaauaugauaaggug

SEQ ID NO: 204 - DNA, 200 bp, Artificial Sequence
  source: /note=“Description of Artificial Sequence: Synthetic polynucleotide”
ttttcacactgtatcccaatgctgccatcattgcaaagattgctgactacagcattgctcagtactgctgtagaatggggataaaaacatcagagggcacgtgccctctgatgtttttatccccattcggttacagtaatgagcaaatgccgagtcagcaagatttgctggttgggcagcattgggatacagtgtgaaaa

SEQ ID NO: 205 - DNA, 165 bp, Artificial Sequence
  source: /note=“Description of Artificial Sequence: Synthetic polynucleotide”
ttgtgaaagaaggacgggtcccttctctccgtcattgcaaagattgctgactacagcattgctcagtactgctgtaatatgctaaatgttacagcaggactgggcagtgccgtagtgtcgaatctttgcaatgaccttctctccgcctgtgactaactgtgccaa

SEQ ID NO: 206 - DNA, 134 bp, Artificial Sequence
  source: /note=“Description of Artificial Sequence: Synthetic polynucleotide”
gtactgagcaatgccctagtcaaagtggacagcaatctttgcaacgatggcagcatcgggatacacctgtgatcacactgtatcccaatgctgccatcattgcaaagattgctgactacagcattgctcagtac

SEQ ID NO: 207 - DNA, 16 bp, Artificial Sequence
  source: /note=“Description of Artificial Sequence: Synthetic oligonucleotide”
attagccatggatgta

SEQ ID NO: 208 - DNA, 135 bp, Artificial Sequence
  source: /note=“Description of Artificial Sequence: Synthetic polynucleotide”
gtactgagcaatgccgtagtcaaagtggacagcaatctttgcaacgatggcagcatcgggatacacctgtgattcacactgtatcccaatgctgccatcattgcaaagattgctgactacagcattgctcagtac

SEQ ID NO: 209 - DNA, 15 bp, Artificial Sequence
  source: /note=“Description of Artificial Sequence: Synthetic oligonucleotide”
tacatggtagcaggg

SEQ ID NO: 210 - DNA, 22 bp, Artificial Sequence
  source: /note=“Description of Artificial Sequence: Synthetic oligonucleotide”
gaagtgaatacatggtagcagg

SEQ ID NO: 211 - DNA, 45 bp, Artificial Sequence
  source: /note=“Description of Artificial Sequence: Synthetic oligonucleotide”
tcattgcaaagattgctgactacagcattgctcagtactgctgta

SEQ ID NO: 212 - RNA, 198 nt, Artificial Sequence
  source: /note=“Description of Artificial Sequence: Synthetic polynucleotide”
aaaagugugacauagguuacgacgguaguaacguuucuuugcugugaugccgugacgaggcauuacgacaucuuaccccuauuuuuguagucucccgugcacgggagacuacaaaaauagggguaagaugucguaaugccucgucacggcaucacagcaaagaaacguuacuaccgucguaaccuaugucacacuuuu

SEQ ID NO: 213 - RNA, 200 nt, Artificial Sequence
  source: /note=“Description of Artificial Sequence: Synthetic polynucleotide”
aacguuucuuugcugugaugccgugacgaggcauuacgacaucuuaccccuauuuuuguagucucccguguggucccaaagcacguggacuucaacggucgaccguugaaguccacgugcuuugggaccacacgggagacuacaaaaauagggguaagaugucguaaugccucgucacggcaucacagcaaagaaacguu

SEQ ID NO: 214 - DNA, 100 bp, Artificial Sequence
  source: /note=“Description of Artificial Sequence: Synthetic polynucleotide”
ctggcaacttcaggtgcacgaaaccctggtgtgccctctgatgtttttatccccattctacagcattacggagcagtgccgtagtgtcgtttctttgcaa

SEQ ID NO: 215 - RNA, 165 nt, Artificial Sequence
  source: /note=“Description of Artificial Sequence: Synthetic polynucleotide”
uugugaaagaaggacgggucgucugguugugucauugcaaagauugcugacuacagcauugcucaguacugcuguaauaugcuaaauguuacagcaguacugagcaaugcuguagucagcaaucuuugcaaugagucugguugugccugugacuaacugugccaa

SEQ ID NO: 216 - DNA, 165 bp, Artificial Sequence
  source: /note=“Description of Artificial Sequence: Synthetic polynucleotide”
ttgtgaaagaaggacgggtcgtctggttgtgtcattgcaaagattgctgactacagcattgctcagtactgctgtaatatgctaaatgttacagcagtactgagcaatgctgtagtcagcaatctttgcaatgagtctggttgtgcctgtgactaactgtgccaa

SEQ ID NO: 217 - RNA, 165 nt, Artificial Sequence
  source: /note=“Description of Artificial Sequence: Synthetic polynucleotide”
uugugaaagaaggacgggucgugcguuguggucauugcaaagauugcugacuacagcauugcucaguacugcuguaauaugcuaaauguuacagcaguacugagcaaugccguagucagcaaucuuugcaaugagugcguuguggccugugacuaacugugccaa

SEQ ID NO: 218 - DNA, 165 bp, Artificial Sequence
  source: /note=“Description of Artificial Sequence: Synthetic polynucleotide”
ttgtgaaagaaggacgggtcgtgcgttgtggtcattgcaaagattgctgactacagcattgctcagtactgctgtaatatgctaaatgttacagcagtactgagcaatgccgtagtcagcaatctttgcaatgagtgcgttgtggcctgtgactaactgtgccaa

SEQ ID NO: 219 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaguacuggguggugccguagucagcaaucuuugcaaugaccucuuguucgccugugacuaacugugccaa

SEQ ID NO: 220 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcagtactgggtggtgccgtagtcagcaatctttgcaatgacctcttgttcgcctgtgactaactgtgccaa

SEQ ID NO: 221 - RNA, 165 nt, Artificial Sequence
  source: /note=“Description of Artificial Sequence: Synthetic polynucleotide”
uugugaaagaaggacgggucccuucucuccgucauugcaaagauugcugacuacagcauugcucaguacugcuguaauaugcuaaauguuacagcaggacugagcagugccguagugucgaaucuuugcaaugaccuucucuccgccugugacuaacugugccaa

SEQ ID NO: 222 - DNA, 165 bp, Artificial Sequence
  source: /note=“Description of Artificial Sequence: Synthetic polynucleotide”
ttgtgaaagaaggacgggtcccttctctccgtcattgcaaagattgctgactacagcattgctcagtactgctgtaatatgctaaatgttacagcaggactgagcagtgccgtagtgtcgaatctttgcaatgaccttctctccgcctgtgactaactgtgccaa

SEQ ID NO: 223 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuaaagcaggacugaguagugccguagucagcaaucuuugcaaugaccucuuguucgccugugacuaacugugccaa

SEQ ID NO: 224 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttaaagcaggactgagtagtgccgtagtcagcaatctttgcaatgacctcttgttcgcctgtgactaactgtgccaa

SEQ ID NO: 225 - RNA, 165 nt, Artificial Sequence
  source: /note=“Description of Artificial Sequence: Synthetic polynucleotide”
uugugaaagaaggacgggucgugguuguucgucauugcaaagauugcugacuacagcauugcucaguacugcuguaauaugcuaaauguuacagcaguacugagcagugccguagugucguuucuuugcaaugagugguuguucgccugugacuaacugugccaa

SEQ ID NO: 226 - DNA, 165 bp, Artificial Sequence
  source: /note=“Description of Artificial Sequence: Synthetic polynucleotide”
ttgtgaaagaaggacgggtcgtggttgttcgtcattgcaaagattgctgactacagcattgctcagtactgctgtaatatgctaaatgttacagcagtactgagcagtgccgtagtgtcgtttctttgcaatgagtggttgttcgcctgtgactaactgtgccaa

SEQ ID NO: 227 - RNA, 165 nt, Artificial Sequence
  source: /note=“Description of Artificial Sequence: Synthetic polynucleotide”
uugugaaagaaggacgggucgugguuguucgucauugcaaagauugcugacuacagcauugcucaguacugcuguaauaugcuaaauguuacagcauuacugagcagugccguagugucguuucuuugcaaugagugguuguucgccugugacuaacugugccaa

SEQ ID NO: 228 - DNA, 165 bp, Artificial Sequence
  source: /note=“Description of Artificial Sequence: Synthetic polynucleotide”
ttgtgaaagaaggacgggtcgtggttgttcgtcattgcaaagattgctgactacagcattgctcagtactgctgtaatatgctaaatgttacagcattactgagcagtgccgtagtgtcgtttctttgcaatgagtggttgttcgcctgtgactaactgtgccaa

SEQ ID NO: 229 - RNA, 165 nt, Artificial Sequence
  source: /note=“Description of Artificial Sequence: Synthetic polynucleotide”
uugugaaagaaggacgggucccuucucuccgucauugcaaagauugcugacuacagcauugcucaguacugcuguaauaugcuaaauguuacagcaggacugaguagugccguagugucgaaucuuugcaaugaccuucucuccgccugugacuaacugugccaa

SEQ ID NO: 230 - DNA, 165 bp, Artificial Sequence
  source: /note=“Description of Artificial Sequence: Synthetic polynucleotide”
ttgtgaaagaaggacgggtcccttctctccgtcattgcaaagattgctgactacagcattgctcagtactgctgtaatatgctaaatgttacagcaggactgagtagtgccgtagtgtcgaatctttgcaatgaccttctctccgcctgtgactaactgtgccaa

SEQ ID NO: 231 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaggacugagcggugccguagucagcaaucuuugcaaugaccucuuguucgccugugacuaacugugccaa

SEQ ID NO: 232 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcaggactgagcggtgccgtagtcagcaatctttgcaatgacctcttgttcgcctgtgactaactgtgccaa

SEQ ID NO: 233 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaguacugagcggugccguagucagcaaucuuugcaaugaccucuuguucgccugugacuaacugugccaa

SEQ ID NO: 234 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcagtactgagcggtgccgtagtcagcaatctttgcaatgacctcttgttcgcctgtgactaactgtgccaa

SEQ ID NO: 235 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaggacugggugaugccguagucagcaaucuuugcaaugaccucuuguucgccugugacuaacugugccaa

SEQ ID NO: 236 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcaggactgggtgatgccgtagtcagcaatctttgcaatgacctcttgttcgcctgtgactaactgtgccaa

SEQ ID NO: 237 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuaaagcaguacugagcggugccguagucagcaaucuuugcaaugaccucuuguucgccugugacuaacugugccaa

SEQ ID NO: 238 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttaaagcagtactgagcggtgccgtagtcagcaatctttgcaatgacctcttgttcgcctgtgactaactgtgccaa

SEQ ID NO: 239 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaguccuggguggugccguagucagcaaucuuugcaaugaccucuuguucgccugugacuaacugugccaa

SEQ ID NO: 240 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcagtcctgggtggtgccgtagtcagcaatctttgcaatgacctcttgttcgcctgtgactaactgtgccaa

SEQ ID NO: 241 - RNA, 165 nt, Artificial Sequence
  source: /note=“Description of Artificial Sequence: Synthetic polynucleotide”
uugugaaagaaggacgggucccuucucuccgucauugcaaagauugcugacuacagcauugcucaguacugcuguaauaugcuaaauguuacagcaggacuggguagugccguagugucgaaucuuugcaaugaccuucucuccgccugugacuaacugugccaa

SEQ ID NO: 242 - DNA, 165 bp, Artificial Sequence
  source: /note=“Description of Artificial Sequence: Synthetic polynucleotide”
ttgtgaaagaaggacgggtcccttctctccgtcattgcaaagattgctgactacagcattgctcagtactgctgtaatatgctaaatgttacagcaggactgggtagtgccgtagtgtcgaatctttgcaatgaccttctctccgcctgtgactaactgtgccaa

SEQ ID NO: 243 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaggacugagcagugccguagucagcaaucuuugcaaugaccucuuguucgccugugacuaacugugccaa

SEQ ID NO: 244 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcaggactgagcagtgccgtagtcagcaatctttgcaatgacctcttgttcgcctgtgactaactgtgccaa

SEQ ID NO: 245 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuaaagcaguacugagcagugccguagucagcaaucuuugcaaugaccucuuguucgccugugacuaacugugccaa

SEQ ID NO: 246 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttaaagcagtactgagcagtgccgtagtcagcaatctttgcaatgacctcttgttcgcctgtgactaactgtgccaa

SEQ ID NO: 247 - RNA, 165 nt, Artificial Sequence
  source: /note=“Description of Artificial Sequence: Synthetic polynucleotide”
uugugaaagaaggacgggucccuucucuccgucauugcaaagauugcugacuacagcauugcucaguacugcuguaauaugcuaaauguuaaagcaggccugagcagugccguagugucgaaucuuugcaaugaccuucucuccgccugugacuaacugugccaa

SEQ ID NO: 248 - DNA, 165 bp, Artificial Sequence
  source: /note=“Description of Artificial Sequence: Synthetic polynucleotide”
ttgtgaaagaaggacgggtcccttctctccgtcattgcaaagattgctgactacagcattgctcagtactgctgtaatatgctaaatgttaaagcaggcctgagcagtgccgtagtgtcgaatctttgcaatgaccttctctccgcctgtgactaactgtgccaa

SEQ ID NO: 249 - RNA, 165 nt, Artificial Sequence
  source: /note=“Description of Artificial Sequence: Synthetic polynucleotide”
uugugaaagaaggacgggucccuucucuccgucauugcaaagauugcugacuacagcauugcucaguacugcuguaauaugcuaaauguuaaagcaggacugggcagugccguagugucgaaucuuugcaaugaccuucucuccgccugugacuaacugugccaa

SEQ ID NO: 250 - DNA, 165 bp, Artificial Sequence
  source: /note=“Description of Artificial Sequence: Synthetic polynucleotide”
ttgtgaaagaaggacgggtcccttctctccgtcattgcaaagattgctgactacagcattgctcagtactgctgtaatatgctaaatgttaaagcaggactgggcagtgccgtagtgtcgaatctttgcaatgaccttctctccgcctgtgactaactgtgccaa

SEQ ID NO: 251 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaggacugggcgaugccguagucagcaaucuuugcaaugaccucuuguucgccugugacuaacugugccaa

SEQ ID NO: 252 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcaggactgggcgatgccgtagtcagcaatctttgcaatgacctcttgttcgcctgtgactaactgtgccaa

SEQ ID NO: 253 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaggacugggcagugccguagucagcaaucuuugcaaugaccucuuguucgccugugacuaacugugccaa

SEQ ID NO: 254 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcaggactgggcagtgccgtagtcagcaatctttgcaatgacctcttgttcgcctgtgactaactgtgccaa

SEQ ID NO: 255 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuaaagcaggacugggcagugccguagucagcaaucuuugcaaugaccucuuguucgccugugacuaacugugccaa

SEQ ID NO: 256 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttaaagcaggactgggcagtgccgtagtcagcaatctttgcaatgacctcttgttcgcctgtgactaactgtgccaa

SEQ ID NO: 257 - RNA, 165 nt, Artificial Sequence
  source: /note=“Description of Artificial Sequence: Synthetic polynucleotide”
uugugaaagaaggacgggucgugguuguucgucauugcaaagauugcugacuacagcauugcucaguacugcuguaauaugcuaaauguuacagcauuacggagcagugccguagugucguuucuuugcaaugagugguuguucgccugugacuaacugugccaa

SEQ ID NO: 258 - DNA, 165 bp, Artificial Sequence
  source: /note=“Description of Artificial Sequence: Synthetic polynucleotide”
ttgtgaaagaaggacgggtcgtggttgttcgtcattgcaaagattgctgactacagcattgctcagtactgctgtaatatgctaaatgttacagcattacggagcagtgccgtagtgtcgtttctttgcaatgagtggttgttcgcctgtgactaactgtgccaa

SEQ ID NO: 259 - RNA, 165 nt, Artificial Sequence
  source: /note=“Description of Artificial Sequence: Synthetic polynucleotide”
uugugaaagaaggacgggucccuucucuccgucauugcaaagauugcugacuacagcauugcucaguacugcuguaauaugcuaaauguuacagcaggccugagcagugccguagugucgaaucuuugcaaugaccuucucuccgccugugacuaacugugccaa

SEQ ID NO: 260 - DNA, 165 bp, Artificial Sequence
  source: /note=“Description of Artificial Sequence: Synthetic polynucleotide”
ttgtgaaagaaggacgggtcccttctctccgtcattgcaaagattgctgactacagcattgctcagtactgctgtaatatgctaaatgttacagcaggcctgagcagtgccgtagtgtcgaatctttgcaatgaccttctctccgcctgtgactaactgtgccaa

SEQ ID NO: 261 - RNA, 165 nt, Artificial Sequence
  source: /note=“Description of Artificial Sequence: Synthetic polynucleotide”
uugugaaagaaggacgggucccuucucuccgucauugcaaagauugcugacuacagcauugcucaguacugcuguaauaugcuaaauguuaaagcaggacuggguagugccguagugucgaaucuuugcaaugaccuucucuccgccugugacuaacugugccaa

SEQ ID NO: 262 - DNA, 165 bp, Artificial Sequence
  source: /note=“Description of Artificial Sequence: Synthetic polynucleotide”
ttgtgaaagaaggacgggtcccttctctccgtcattgcaaagattgctgactacagcattgctcagtactgctgtaatatgctaaatgttaaagcaggactgggtagtgccgtagtgtcgaatctttgcaatgaccttctctccgcctgtgactaactgtgccaa

SEQ ID NO: 263 - RNA, 165 nt, Artificial Sequence
  source: /note=“Description of Artificial Sequence: Synthetic polynucleotide”
uugugaaagaaggacgggucccuucucuccgucauugcaaagauugcugacuacagcauugcucaguacugcuguaauaugcuaaauguuacagcaguacugagcagugccguagugucgaaucuuugcaaugaccuucucuccgccugugacuaacugugccaa

SEQ ID NO: 264 - DNA, 165 bp, Artificial Sequence
  source: /note=“Description of Artificial Sequence: Synthetic polynucleotide”
ttgtgaaagaaggacgggtcccttctctccgtcattgcaaagattgctgactacagcattgctcagtactgctgtaatatgctaaatgttacagcagtactgagcagtgccgtagtgtcgaatctttgcaatgaccttctctccgcctgtgactaactgtgccaa

SEQ ID NO: 265 - RNA, 165 nt, Artificial Sequence
  source: /note=“Description of Artificial Sequence: Synthetic polynucleotide”
uugugaaagaaggacgggucgugcguuguggucauugcaaagauugcugacuacagcauugcucaguacugcuguaauaugcuaaauguuacagcaauaccgagcagugccguagucagcaaucuuugcaaugagugcguuguggccugugacuaacugugccaa

SEQ ID NO: 266 - DNA, 165 bp, Artificial Sequence
  source: /note=“Description of Artificial Sequence: Synthetic polynucleotide”
ttgtgaaagaaggacgggtcgtgcgttgtggtcattgcaaagattgctgactacagcattgctcagtactgctgtaatatgctaaatgttacagcaataccgagcagtgccgtagtcagcaatctttgcaatgagtgcgttgtggcctgtgactaactgtgccaa

SEQ ID NO: 267 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaguacugggcggugccguagucagcaaucuuugcaaugaccucuuguucgccugugacuaacugugccaa

SEQ ID NO: 268 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcagtactgggcggtgccgtagtcagcaatctttgcaatgacctcttgttcgcctgtgactaactgtgccaa

SEQ ID NO: 269 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaggacuggguggugccguagucagcaaucuuugcaaugaccucuuguucgccugugacuaacugugccaa

SEQ ID NO: 270 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcaggactgggtggtgccgtagtcagcaatctttgcaatgacctcttgttcgcctgtgactaactgtgccaa

SEQ ID NO: 271 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuaaagcaggacugagcagugccguagucagcaaucuuugcaaugaccucuuguucgccugugacuaacugugccaa

SEQ ID NO: 272 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttaaagcaggactgagcagtgccgtagtcagcaatctttgcaatgacctcttgttcgcctgtgactaactgtgccaa

SEQ ID NO: 273 - RNA, 165 nt, Artificial Sequence
  source: /note=“Description of Artificial Sequence: Synthetic polynucleotide”
uugugaaagaaggacgggucccuucucuccgucauugcaaagauugcugacuacagcauugcucaguacugcuguaauaugcuaaauguuaaagcaggacugaguagugccguagugucgaaucuuugcaaugaccuucucuccgccugugacuaacugugccaa

SEQ ID NO: 274 - DNA, 165 bp, Artificial Sequence
  source: /note=“Description of Artificial Sequence: Synthetic polynucleotide”
ttgtgaaagaaggacgggtcccttctctccgtcattgcaaagattgctgactacagcattgctcagtactgctgtaatatgctaaatgttaaagcaggactgagtagtgccgtagtgtcgaatctttgcaatgaccttctctccgcctgtgactaactgtgccaa

SEQ ID NO: 275 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaguacugaguggugccguagucagcaaucuuugcaaugaccucuuguucgccugugacuaacugugccaa

SEQ ID NO: 276 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcagtactgagtggtgccgtagtcagcaatctttgcaatgacctcttgttcgcctgtgactaactgtgccaa

SEQ ID NO: 277 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaggacugggcggugccguagucagcaaucuuugcaaugaccucuuguucgccugugacuaacugugccaa

SEQ ID NO: 278 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcaggactgggcggtgccgtagtcagcaatctttgcaatgacctcttgttcgcctgtgactaactgtgccaa

SEQ ID NO: 279 - RNA, 165 nt, Artificial Sequence
  source: /note=“Description of Artificial Sequence: Synthetic polynucleotide”
uugugaaagaaggacgggucccucuuguucgucauugcaaagauugcugacuacagcauugcucaguacugcuguaauaugcuaaauguuacagcaguccugggcggugccguagucagcaaucuuugcaaugaccucuuguucgccugugacuaacugugccaa

SEQ ID NO: 280 - DNA, 165 bp, Artificial Sequence
  source: /note=“Description of Artificial Sequence: Synthetic polynucleotide”
ttgtgaaagaaggacgggtccctcttgttcgtcattgcaaagattgctgactacagcattgctcagtactgctgtaatatgctaaatgttacagcagtcctgggcggtgccgtagtcagcaatctttgcaatgacctcttgttcgcctgtgactaactgtgccaa

SEQ ID NO: 281 - RNA, 165 nt, Artificial Sequence
  source: /note=“Description of Artificial Sequence: Synthetic polynucleotide”
uugugaaagaaggacgggucccuucucuccgucauugcaaagauugcugacuacagcauugcucaguacugcuguaauaugcuaaauguuaaagcaguacugagcagugccguagugucgaaucuuugcaaugaccuucucuccgccugugacuaacugugccaa

SEQ ID NO: 282 - DNA, 165 bp, Artificial Sequence
  source: /note=“Description of Artificial Sequence: Synthetic polynucleotide”
ttgtgaaagaaggacgggtcccttctctccgtcattgcaaagattgctgactacagcattgctcagtactgctgtaatatgctaaatgttaaagcagtactgagcagtgccgtagtgtcgaatctttgcaatgaccttctctccgcctgtgactaactgtgccaa

SEQ ID NO: 283 - RNA, 165 nt, Artificial Sequence
  source: /note=“Description of Artificial Sequence: Synthetic polynucleotide”
uugugaaagaaggacgggucccuucucuccgucauugcaaagauugcugacuacagcauugcucaguacugcuguaauaugcuaaauguuaaagcaggacugagcagugccguagugucgaaucuuugcaaugaccuucucuccgccugugacuaacugugccaa

SEQ ID NO: 284 - DNA, 165 bp, Artificial Sequence
  source: /note=“Description of Artificial Sequence: Synthetic polynucleotide”
ttgtgaaagaaggacgggtcccttctctccgtcattgcaaagattgctgactacagcattgctcagtactgctgtaatatgctaaatgttaaagcaggactgagcagtgccgtagtgtcgaatctttgcaatgaccttctctccgcctgtgactaactgtgccaa